(19) United States
(12) Patent Application Publication
(10) Pub. No.: US 2023/0173104 A1
(43) Pub. Date: Jun. 8, 2023

(54) LNP COMPOSITIONS COMPRISING AN MRNA THERAPEUTIC AND AN EFFECTOR MOLECULE

(71) Applicant: ModernaTX, Inc., Cambridge, MA (US)

(72) Inventors: Ruchi Jain, Brookline, MA (US); Alicia Anne Bicknell, Somerville, MA (US)

(21) Appl. No.: 17/924,456

(22) PCT Filed: May 14, 2021
(86) PCT No.: PCT/US2021/032438

§ 371 Date: Nov. 10, 2022

Related U.S. Application Data
(60) Provisional application No. 63/024,862, filed on May 14, 2020.
(60) Provisional application No. 63/183,119, filed on May 3, 2021.

(51) Int. Cl. A61K 48/00; A61K 9/51; A61K 38/17; A61K 38/16

(52) U.S. Cl. CPC A61K 48/0058; A61K 9/5123; A61K 48/0033; A61K 38/1709; A61K 9/5146; A61K 38/162

(57) ABSTRACT

The disclosure features LNP compositions and systems comprising a therapeutic payload or prophylactic payload, a binding element, a tether molecule and/or an effector molecule and uses thereof. The LNP compositions or systems of the present disclosure comprise: (a) a first polynucleotide (e.g., mRNA) comprising: (1) a sequence encoding a therapeutic payload or prophylactic payload, and (2) a binding element; and (b) a second polynucleotide (e.g., mRNA) comprising a sequence encoding: (1) an effector molecule, and/or (2) a polypeptide that recognizes the binding element (a tether molecule). Such compositions or systems can: increase the level and/or activity of the therapeutic payload or prophylactic payload, e.g., increase the level, stability and/or activity of the mRNA encoding the therapeutic payload or prophylactic payload. Also disclosed herein are methods of treating a disorder, or for modulating an immune response in a subject using the disclosed LNP compositions or systems.

### CROSS-REFERENCE TO RELATED APPLICATIONS

This application claims the benefit of priority of U.S. Provisional Appl. No. 63/024,862, filed May 14, 2020, and U.S. Provisional Appl. No. 63/183,119, filed May 3, 2021, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on May 14, 2021, is named 45817-0103W01 SL.txt and is 475,291 bytes in size.

### BACKGROUND OF THE DISCLOSURE

Efforts to increase mRNA potency have focused on generating canonical linear mRNAs with optimal sequence design for the untranslated regions (UTRs) and open reading frame (ORFs). Recent advances have added end-protection where modified caps and tails render mRNAs more resistant to the cellular degradation machinery. However, there is no indication that the maximum potency possible has been achieved with these efforts, with respect to peak or duration of expression. This is particularly true in the case of mRNA therapeutics. Current approaches are focused on modifying the mRNAs themselves. Therefore, there is a need to further improve peak and/or duration of mRNA expression by exploiting RNA biology.

### SUMMARY OF THE DISCLOSURE

The present disclosure provides, inter alia, lipid nanoparticle (LNP) compositions or systems comprising a therapeutic payload or prophylactic payload, a binding element, a tether molecule and/or an effector molecule and uses thereof. The LNP compositions or systems of the present disclosure comprise: (a) a first polynucleotide (e.g., mRNA) comprising: (1) a sequence encoding a therapeutic payload or prophylactic payload, and (2) a binding element; and (b) a second polynucleotide (e.g., mRNA) comprising a sequence encoding: (1) an effector molecule, and/or (2) a polypeptide that binds to, e.g., recognizes the binding element (a tether molecule). In some embodiments, the effector molecule recognizes and binds to the binding element. In an embodiment, the first polynucleotide and the second polynucleotide are disposed in the same or different polynucleotides. In an embodiment, a system disclosed herein is formulated as an LNP. In an embodiment, the LNP comprising the first polynucleotide and the LNP comprising the second polynucleotide are formulated in the same LNP. In an embodiment, the LNP comprising the first polynucleotide and the LNP comprising the second polynucleotide are formulated in different LNPs. In an aspect, the LNP compositions or systems of the present disclosure can: increase the level, duration of expression, and/or activity of the therapeutic payload or prophylactic payload, e.g., increase the level, duration of expression, and/or activity of the mRNA encoding the therapeutic payload or prophylactic payload, or increase the level, duration of expression and/or activity of the therapeutic payload or prophylactic payload. Also disclosed herein are methods of using an LNP composition or system comprising a therapeutic payload or prophylactic payload, a binding element, a tether molecule and/or an effector molecule, for treating a disease or disorder, or for promoting a desired biological effect in a subject, e.g., for modulating an immune response in a subject. Additional aspects of the disclosure are described in further detail below.

In an aspect, disclosed herein is a lipid nanoparticle (LNP) composition comprising: (a) a first polynucleotide comprising: (1) a sequence encoding a polypeptide, and (2) a binding element; and (b) a second polynucleotide comprising a sequence encoding: (1) an effector molecule, and (2) a polypeptide that binds to, e.g., recognizes, the binding element (a tether molecule).

In an embodiment, the polypeptide of (a) does not comprise a reporter protein. In another embodiment, the polypeptide of (a) encodes a peptide or polypeptide having a desirable biologic effect, e.g., a therapeutic protein.

In an aspect, disclosed herein is a lipid nanoparticle (LNP) composition comprising:

(a) a first polynucleotide comprising: (1) a sequence encoding a polypeptide, and (2) a binding element; and (b) a second polynucleotide comprising a sequence encoding: (1) an effector molecule. In an embodiment, the effector molecule further comprises a polypeptide that binds to, e.g., recognizes, the binding element (a tether molecule).

In an embodiment, the polypeptide of (a) does not comprise a reporter protein. In another embodiment, the polypeptide of (a) encodes a peptide or polypeptide having a desirable biologic effect, e.g., a therapeutic protein.

In an embodiment the effector molecule polypeptide comprising a tether molecule comprises a first domain which modulates a parameter of, e.g., level and/or activity of: an RNA (e.g., an mRNA); or a protein encoded by the RNA. In an embodiment, the parameter comprises one, two, three or all of: (1) mRNA level and/or activity and/or subcellular localization (e.g., half-life and/or expression); (2) protein level and/or activity (e.g., half-life and/or expression); (3) protein translation rate or (4) protein localization, e.g., location. In an embodiment the effector molecule polypeptide comprising a tether molecule comprises a second domain which binds to, e.g., recognizes, the binding element (a tether molecule).

In an embodiment the effector molecule comprising the tether molecule comprises a polypeptide comprising the first domain and the second domain. In an embodiment, the first and second domains are operatively linked.

In an embodiment, in the second polynucleotide encoding the effector molecule which further comprises a tether molecule, the nucleotide sequence encoding the effector molecule is upstream of the nucleotide sequence encoding the tether molecule. In an embodiment, the nucleotide sequence encoding the effector molecule is downstream of the nucleotide sequence encoding the tether molecule. In an embodiment, the nucleotide sequence encoding the effector molecule is separated from the nucleotide sequence encoding the tether molecule by a protease cleavage site (e.g., a P2A, T2A, E2A, or TPE (P2A-T2A-E2A) site) or an internal ribosomal entry site.

In an embodiment, in the second polynucleotide encoding the effector molecule which further comprises a tether molecule, the nucleotide sequence encoding the effector molecule is adjacent to the nucleotide sequence encoding the tether molecule.

In another aspect, disclosed herein is a lipid nanoparticle (LNP) composition comprising: (a) a first polynucleotide comprising: (1) a sequence encoding a therapeutic payload or prophylactic payload, and (2) a binding element; and (b) a second polynucleotide comprising a sequence encoding: (1) an effector molecule, and (2) a polypeptide that binds to, e.g., recognizes, the binding element (a tether molecule). In an embodiment, (a) and (b) each comprise an mRNA.

In an embodiment, the first polynucleotide and the second polynucleotide are disposed in the same polynucleotide.

In an embodiment, the first polynucleotide and the second polynucleotide, in no particular order, are separated by a protease cleavage site (e.g., a P2A, T2A, E2A, or TPE (P2A-T2A-E2A) site) or an internal ribosomal entry site.

In an embodiment, the first polynucleotide and the second polynucleotide are disposed in different polynucleotides.

In an embodiment, (a) and (b) are formulated as LNPs, e.g., formulated as the same LNP. In an embodiment, (a) and (b) are formulated as different LNPs.

In another aspect, disclosed herein is a lipid nanoparticle (LNP) composition comprising: (a) a first polynucleotide comprising: (1) a sequence encoding a therapeutic payload or prophylactic payload, and (2) a binding element; and (b) a second polynucleotide comprising a sequence encoding: (1) an effector molecule. In an embodiment, the effector molecule further comprises a polypeptide that binds to, e.g., recognizes, the binding element (a tether molecule).

In an embodiment the effector molecule polypeptide comprising a tether molecule comprises a first domain which modulates a parameter of, e.g., level and/or activity of: an RNA (e.g., an mRNA); or a protein encoded by the RNA. In an embodiment, the parameter comprises one, two, three or all of: (1) mRNA level and/or activity and/or subcellular localization (e.g., half-life and/or expression); (2) protein level and/or activity (e.g., half-life and/or expression); (3) protein translation rate or (4) protein localization, e.g., location. In an embodiment the effector molecule polypeptide comprising a tether molecule comprises a second domain which binds to, e.g., recognizes, the binding element (a tether molecule).

In an embodiment the effector molecule comprising the tether molecule comprises a polypeptide comprising the first domain and the second domain. In an embodiment, the first and second domains are operatively linked.

In an embodiment, in the second polynucleotide encoding the effector molecule which further comprises a tether molecule, the nucleotide sequence encoding the effector molecule is upstream of the nucleotide sequence encoding the tether molecule. In an embodiment, the nucleotide sequence encoding the effector molecule is downstream of the nucleotide sequence encoding the tether molecule. In an embodiment, the nucleotide sequence encoding the effector molecule is separated from the nucleotide sequence encoding the tether molecule by a protease cleavage site (e.g., a P2A, T2A, E2A, or TPE (P2A-T2A-E2A) site) or an internal ribosomal entry site.

In an embodiment, in the second polynucleotide encoding the effector molecule which further comprises a tether molecule, the nucleotide sequence encoding the effector molecule is adjacent to the nucleotide sequence encoding the tether molecule.

In an embodiment, (a) and (b) each comprise an mRNA.

In an embodiment, the first polynucleotide and the second polynucleotide are disposed in the same polynucleotide.

In an embodiment, the first polynucleotide and the second polynucleotide are separated by a protease cleavage site (e.g., a P2A, T2A, E2A, or TPE (P2A-T2A-E2A) site) or an internal ribosomal entry site.

In an embodiment, the first polynucleotide and the second polynucleotide are disposed in different polynucleotides.

In an embodiment, (a) and (b) are formulated as LNPs, e.g., formulated as the same LNP. In an embodiment, (a) and (b) are formulated as different LNPs.

In an aspect, the disclosure provides a system comprising: (a) a first polynucleotide comprising: (1) a sequence encoding a polypeptide, and (2) a binding element; and (b) a second polynucleotide comprising a sequence encoding: (1) an effector, and a (2) polypeptide that binds to, e.g., recognizes, the binding element (a tether molecule).

In an embodiment, the polypeptide of (a) does not comprise a reporter protein.

In yet another aspect, disclosed herein is a system comprising: (a) a first polynucleotide comprising: (1) a sequence encoding a therapeutic payload or prophylactic payload, and (2) a binding element; and/or (b) a second polynucleotide comprising a sequence encoding: (1) an effector molecule, and a (2) polypeptide that recognizes the binding element (a tether molecule). In an embodiment, (a) and (b) each comprise an mRNA.

In an embodiment, the system comprises (a).

In an embodiment, the system comprises (b).

In an embodiment, the system comprises (a) and (b).

In an embodiment, the first polynucleotide and the second polynucleotide are disposed in the same polynucleotide.

In an embodiment, the first polynucleotide and the second polynucleotide are separated by a protease cleavage site (e.g., a P2A, T2A, E2A, or TPE (P2A-T2A-E2A) site) or an internal ribosomal entry site.

In an embodiment, the first polynucleotide and the second polynucleotide are disposed in different polynucleotides.

In an embodiment, the therapeutic payload or prophylactic payload is not a reporter protein.

In another aspect, disclosed herein is a system comprising: (a) a first polynucleotide comprising: (1) a sequence encoding a therapeutic payload or prophylactic payload, and (2) a binding element; and/or (b) a second polynucleotide comprising a sequence encoding: (1) an effector molecule In an embodiment, the effector molecule further comprises a polypeptide that binds to, e.g., recognizes, the binding element (a tether molecule).

In an embodiment the effector molecule polypeptide comprising a tether molecule comprises a first domain which modulates a parameter of, e.g., level and/or activity of: an RNA (e.g., an mRNA); or a protein encoded by the RNA. In an embodiment, the parameter comprises one, two, three or all of: (1) mRNA level and/or activity and/or subcellular localization (e.g., half-life and/or expression); (2) protein level and/or activity (e.g., half-life and/or expression); (3) protein translation rate or (4) protein localization, e.g., location. In an embodiment the effector molecule polypeptide comprising a tether molecule comprises a second domain which binds to, e.g., recognizes, the binding element (a tether molecule).

In an embodiment the effector molecule comprising the tether molecule comprises a polypeptide comprising the first domain and the second domain. In an embodiment, the first and second domains are operatively linked.

In an embodiment, in the second polynucleotide encoding the effector molecule which further comprises a tether molecule, the nucleotide sequence encoding the effector molecule is upstream of the nucleotide sequence encoding the tether molecule. In an embodiment, the nucleotide sequence encoding the effector molecule is downstream of the nucleotide sequence encoding the tether molecule. In an embodiment, the nucleotide sequence encoding the effector molecule is separated from the nucleotide sequence encoding the tether molecule by a protease cleavage site (e.g., a P2A, T2A, E2A, or TPE (P2A-T2A-E2A) site) or an internal ribosomal entry site.

In an embodiment, in the second polynucleotide encoding the effector molecule which further comprises a tether molecule, the nucleotide sequence encoding the effector molecule is adjacent to the nucleotide sequence encoding the tether molecule.

In an embodiment, (a) and (b) each comprise an mRNA.

In an embodiment, the system comprises (a).

In an embodiment, the system comprises (b).

In an embodiment, the system comprises (a) and (b).

In an embodiment, the first polynucleotide and the second polynucleotide are disposed in the same polynucleotide.

In an embodiment, the first polynucleotide and the second polynucleotide are separated by a protease cleavage site (e.g., a P2A, T2A, E2A, or TPE (P2A-T2A-E2A) site) or an internal ribosomal entry site.

In an embodiment, the first polynucleotide and the second polynucleotide are disposed in different polynucleotides.

In an embodiment, the therapeutic payload or prophylactic payload is not a reporter protein.

In an embodiment of any of the systems disclosed herein, the system comprises less than 5%, 10%, 15%, 20%, 25%, or 50% of a cellular impurity, e.g., a cellular component, e.g., a membrane, protein or lipid derived from a cellular extract.

In an embodiment of any of the systems disclosed herein, at least one of (a) or (b) is formulated as an LNP.

In an embodiment of any of the systems disclosed herein, (a) is formulated as an LNP.

In an embodiment of any of the systems disclosed herein, (b) is formulated as an LNP.

In an embodiment of any of the systems disclosed herein, (a) and (b) both are formulated as LNPs, e.g., the same LNP or different LNPs.

In an embodiment of any of the systems disclosed herein, (a) formulated as an LNP is in a first composition. In an embodiment, (b) formulated as an LNP is in a second composition. In an embodiment, (a) formulated as an LNP and (b) formulated as an LNP are in separate compositions. In an embodiment, (a) formulated as an LNP and (b) formulated as an LNP are in the same composition.

In an aspect, provided herein is a system comprising: (a) a first polynucleotide comprising: (1) a sequence encoding a therapeutic payload or prophylactic payload, and (2) a binding element comprising an MS2 sequence, e.g., 6 MS2 sequences of 19 nucleotides separated by spacers of 20 nucleotides in length; (b) a second polynucleotide comprising a sequence encoding: (1) an effector molecule comprising eIF4G, e.g., wildtype eIF4G, a variant or a fragment thereof; and (2) a tether molecule comprising MBP, e.g., wildtype MBP, a variant or fragment thereof.

In another aspect, provided herein is a pharmaceutical composition comprising a system, or LNP composition of disclosed herein.

In an aspect, the disclosure provides, a cell comprising a system, or LNP composition disclosed herein. In an embodiment, the cell has been contacted with the system, or LNP composition. In an embodiment, the cell is contacted with the system, or LNP composition in vivo. In an embodiment, the cell is contacted with the system, or LNP composition in vitro. In an embodiment, the cell is contacted with the system, or LNP composition ex vivo. In an embodiment, the cell is maintained under conditions sufficient to allow for expression of one or both polynucleotides of the system, or LNP composition.

In an aspect, provided herein is a method of increasing expression of a therapeutic payload or prophylactic payload in a cell, comprising administering to the cell a system, or LNP composition disclosed herein. In one embodiment, the cell is present in a subject.

In a related aspect, provided herein is a system or LNP composition for use in a method of increasing expression of a therapeutic payload or prophylactic payload in a cell.

In another aspect, the disclosure provides a method of increasing expression of a therapeutic payload or prophylactic payload, in a subject, comprising administering to the subject an effective amount of a system or LNP composition disclosed herein.

In a related aspect, provided herein is a system or LNP composition for use in a method of increasing expression of a therapeutic payload or prophylactic payload in a subject.

In yet another aspect, provided herein is a method of delivering a system, or LNP composition disclosed herein.

In a related aspect, provided herein is a system or LNP composition for use in a method of delivering the system or LNP composition to a cell.

In an embodiment, the method or use, comprises contacting the cell in vitro, in vivo or ex vivo with the system or LNP composition.

In an aspect, the disclosure provides a method of delivering a system or LNP composition disclosed herein to a subject having a disease or disorder, e.g., as described herein.

In a related aspect, provided herein is a system or LNP composition for use in a method of delivering the system or LNP composition to a subject having a disease or disorder, e.g., as described herein.

In another aspect, provided herein is a method of modulating an immune response in a subject, comprising administering to the subject in need thereof an effective amount of a system, or LNP composition disclosed herein.

In a related aspect, provided herein is a system or LNP composition for use in a method of modulating an immune response in a subject, comprising administering to the subject an effective amount of the system, or LNP composition.

In an aspect, provided herein is a method of treating, preventing, or preventing a symptom of, a disease or disorder comprising administering to a subject in need thereof an effective amount of a system, or LNP composition disclosed herein.

In a related aspect, provided herein is a system or LNP composition for use in a method of treating, preventing, or preventing a symptom of, a disease or disorder in a subject, comprising administering to the subject in need thereof an effective amount of the system, or LNP composition.

In an embodiment, the LNP composition comprises: (a) a first polynucleotide comprising: (1) a sequence encoding a therapeutic payload or prophylactic payload, and (2) a binding element; and (b) a second polynucleotide comprising a sequence encoding: (1) an effector molecule, and (2) a polypeptide that recognizes the binding element (a tether molecule). In an embodiment, (a) and (b) each comprise an mRNA.

In an embodiment, the system comprises: (a) a first polynucleotide comprising: (1) a sequence encoding a therapeutic payload or prophylactic payload, and (2) a binding element; and (b) a second polynucleotide comprising a sequence encoding: (1) an effector molecule, and (2) a polypeptide that recognizes the binding element (a tether molecule). In an embodiment, (a) and (b) each comprise an mRNA.

In an embodiment, the first polynucleotide and/or the second polynucleotide of the system is formulated as an LNP. In an embodiment, the first polynucleotide of the system is formulated as an LNP. In an embodiment, the second polynucleotide of the system is formulated as an LNP. In an embodiment, both the first and the second polynucleotides of the system are formulated as LNPs.

In an embodiment, the LNP comprising the first polynucleotide is the same as the LNP comprising the second polynucleotide. In an embodiment, the LNP comprising the first polynucleotide is different from the LNP comprising the second polynucleotide.

In an embodiment, the LNP comprising the first polynucleotide is in a first composition. In an embodiment, the LNP comprising the second polynucleotide is in a second composition. In an embodiment, the LNP comprising the first polynucleotide and the LNP comprising the second polynucleotide are in the same composition. In an embodiment, the LNP comprising the first polynucleotide and the LNP comprising the second polynucleotide are in different compositions.

In an embodiment, the LNP comprising (a) and the LNP comprising (b) are administered simultaneously, e.g., substantially simultaneously.

In an embodiment, the LNP comprising (a) and the LNP comprising (b) are administered sequentially.

In an embodiment, the LNP comprising (a) is administered first.

In an embodiment, the LNP comprising (a) is administered first followed by administration of the LNP comprising (b).

In an embodiment, the LNP comprising (b) is administered first.

In an embodiment, the LNP comprising (b) is administered first followed by administration of the LNP comprising (a).

In some embodiments of any of the methods disclosed herein, the LNP composition comprises: (i) an ionizable lipid, e.g., an amino lipid; (ii) a sterol or other structural lipid; (iii) a non-cationic helper lipid or phospholipid; and (iv) a PEG-lipid. In some embodiments, the ionizable lipid comprises a compound of Formula (IIa). In some embodiments, the ionizable lipid comprises a compound of Formula (IIe).

Additional features of any of the LNP compositions, pharmaceutical composition comprising said LNPs, methods or compositions for use disclosed herein include the following embodiments.

In some embodiments of any of the systems, LNP compositions, methods or uses disclosed herein, the tether molecule of the second polynucleotide comprises an RNA binding protein or a fragment thereof, which binds to, e.g., recognizes, the binding element of the first polynucleotide. In some embodiments, the binding element of the first polynucleotide is situated upstream (5′) or downstream (3′) or in the open reading frame of the sequence encoding the therapeutic payload or prophylactic payload. In some embodiments, the binding element of the first polynucleotide is situated upstream (5′) or downstream (3′) of a 5′ UTR of the first polynucleotide. In some embodiments, the binding element of the first polynucleotide is situated upstream (5′) or downstream (3′) of a 3′ UTR of the first polynucleotide. In some embodiments, the binding element of the first polynucleotide is situated downstream of a 3′ UTR of the first polynucleotide.

In some embodiments, the binding element of the first polynucleotide is bound by the tether molecule of the second polynucleotide, e.g., a tether molecule provided in Table 1, e.g., MBP, PCP, Lambda N, U1A or PUF, or 15.5 kd or a variant or fragment thereof. In some embodiments, the binding element comprises a sequence which is bound, e.g., recognized, by the tether molecule. In some embodiments, the binding element comprises a sequence comprising a structure that is bound, e.g., recognized, by the tether molecule.

In some embodiments, the binding element is chosen from a binding element provided in Table 1, e.g., MS2, PP7, BoxB, U1A hairpin or PRE or kink-turn, or a variant or fragment thereof. In some embodiments, the binding element is MS2. In some embodiments, the binding element is PP7. In some embodiments, the binding element is BoxB. In some embodiments, the binding element is U1A hairpin. In some embodiments, the binding element is PRE. In some embodiments, the binding element is kink-turn.

In some embodiments, when the binding element is MS2 (e.g., wildtype MS2, or a variant or fragment thereof) the tether molecule is MBP (e.g., wildtype MBP, a variant or fragment thereof).

In some embodiments, when the binding element is PP7 (e.g., wildtype PP7, or a variant or fragment thereof) the tether molecule is PCP (e.g., wildtype PCP, or a variant or fragment thereof).

In some embodiments, when the binding element is BoxB (e.g., wildtype BoxB, or a variant or fragment thereof) the tether molecule is Lambda N (e.g., wildtype Lambda N, or a variant or fragment thereof).

In some embodiments, when the binding element is U1A hairpin (e.g., wildtype U1A hairpin, or a variant or fragment thereof) the tether molecule is U1A (e.g., wildtype U1A, or a variant or fragment thereof).

In some embodiments, when the binding element is PRE (e.g., wildtype PRE, or a variant or fragment thereof) the tether molecule is PUF (e.g., wildtype PUF, or a variant or fragment thereof).

In some embodiments, when the binding element is a kink-turn forming sequence and the tether molecule is 15.5 kd (e.g., wildtype 15.5 kd, or a variant or fragment thereof).

In some embodiments, the binding element comprises a sequence comprising 5, 6, 7, 8, 9, 10, 11, 12, 13, 14, 15, 16, 17, 18, 19, 20, 21, 22, 23, 24, 25, 30, 40, 50, 60, 70, 80, 90 or 100 nucleotides. In some embodiments, the binding element comprises a sequence comprising about 5-100, about 5-90, about 5-80, about 5-70, about 5-60, about 5-50, about 5-40, about 5-30, about 5-25, about 5-20, about 5-19, about 5-18, about 5-17, about 5-16, about 5-15, about 5-14, about 5-13, about 5-12, about 5-11, about 5-10, about 5-9, about 5-8, about 5-7 or about 5-6 nucleotides. In some embodiments, the binding element comprises a sequence comprising about 5-100, about 6-100, about 7-100, about 8-100, about 9-100, about 10-100, about 11-100, about 12-100, about 13-100, about 14-100, about 15-100, about 16-100, about 17-100, about 18-100, about 19-100, about 20-100, about 21-100, about 22-100, about 23-100, about 24-100, about 25-100, about 30-100, about 40-100, about 50-100, about 60-100, about 70-100, about 80-100, or about 90-100 nucleotides. In some embodiments, the binding element comprises a sequence comprising about 5-100, about 6-90, about 7-80, about 8-70, about 9-60, about 10-50, about 11-40, about 12-30, about 13-25, about 14-24, about 15-23, about 16-22, about 17-21, or about 18-20 nucleotides. In some embodiments, the binding element comprises a sequence comprising 19 nucleotides, e.g., a binding element nucleotide sequence provided in Table 2 or a sequence with at least 80%, 85%, 90%, 95%, 96%, 97%, 98%, 99% or 100% identity thereof

In some embodiments of any of the systems, LNP compositions, methods or uses disclosed herein, the binding element comprises at least 1, 2, 3, 4, 5, 6, 7, 8, 9, 10, 11, 12, 13, 14, 15, 20 or 30 repeats of the sequence bound by the tether molecule of the second polynucleotide. In some embodiments, the binding element comprises no more than 80, 70, 60, 50, 40 or 30 repeats of the sequence bound by the tether molecule of the second polynucleotide. In some embodiments, n the binding element comprises about 1-30, about 1-20, about 1-10, about 1-9, about 1-8, about 1-7, about 1-6, about 1-5, about 1-4, about 1-3, or about 1-2 repeats of the sequence bound by the tether molecule of the second polynucleotide. In some embodiments, the binding element comprises about 1-30, about 2-30, about 3-30, about 4-30 about, 5-30 about, 6-30, about 7-30, about 8-30, about 9-30, about 10-30, about 11-30, about 12-30, about 13-30, about 14-30, about 15-30, or about 20-30 repeats of the sequence bound by the tether molecule of the second polynucleotide. In some embodiments, the binding element comprises about 1-30, about 2-20, about 3-15, about 4-14, about 5-13, about 6-12, about 7-11, or about 8-10 repeats of the sequence bound by the tether molecule of the second polynucleotide. In some embodiments, the binding element comprises 6 repeats of the sequence bound by the tether molecule of the second polynucleotide.

In some embodiments of any of the systems, LNP compositions, methods or uses disclosed herein, each repeat is separated by a spacer sequence comprising 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90 or 100 nucleotides. In some embodiments, the spacer sequence comprises about 1-100, about 1-90, about 1-80, about 1-70, about 1-60, about 1-50, about 1-40, about 1-30, about 1-25, about 1-20, about 1-19, about 1-18, about 1-17, about 1-16, about 1-15, about 1-14, about 1-13, about 1-12, about 1-11, about 1-10, about 1-9, about 1-8, about 1-7, about 1-6, about 1-5, about 1-4, about 1-3, or about 1-2 nucleotides. In some embodiments, the spacer sequence comprises about 1-100, about 2-100, about 3-100, about 4-100, about 5-100, about 6-100, about 7-100, about 8-100, about 9-100, about 10-100, about 11-100, about 12-100, about 13-100, about 14-100, about 15-100, about 16-100, about 17-100, about 18-100, about 19-100, about 20-100, about 21-100, about 22-100, about 23-100, about 24-100, about 25-100, about 30-100, about 40-100, about 50-100, about 60-100, about 70-100, about 80-100, or about 90-100 nucleotides. In some embodiments, the spacer sequence comprises about 1-100, about 2-90, about 3-80, about 4-70, about 5-60, about 6-50, about 7-40, about 8-40, about 9-30, about 10-25, about 11-24, about 12-23, about 13-22, about 14-21, about 15-20, about 16-19, about 17-18 nucleotides. In some embodiment, the spacer sequence comprises 20 nucleotides, e.g., a spacer sequence provided in Table 2 or a sequence with at least 80%, 85%, 90%, 95%, 96%, 97%, 98%, 99% or 100% identity thereof.

In some embodiments of any of the systems, LNP compositions, methods or uses disclosed herein, the effector molecule is chosen from: a translation factor, a splicing factor, an RNA stabilizing factor, an RNA editing factor, an RNA-binding factor, an RNA localizing factor, or a combination thereof. In some embodiments, the effector molecule is a translation factor, e.g., a translation factor provided in Table 4, e.g., eIF4G; Poly A binding protein (PABP); eIF3d or a component thereof; Daz1, or a fragment, or variant or combination thereof.

In some embodiments, the effector molecule is a translation factor which modulates, e.g., facilitates, ribosome binding, e.g., recruitment, pre-initiation complex formation, or RNA unwinding. In some embodiments, the effector molecule comprises eIF4G, e.g., wildtype eIF4G, a variant of eIF4G, or a fragment thereof.

In some embodiments, the effector molecule comprises wildtype eIF4G. In some embodiments, wildtype eIF4G comprises a sequence of about 1600 amino acids. In some embodiments, the effector molecule comprises a fragment of eIF4G, e.g., as disclosed herein. In some embodiments, the eIF4G fragment retains ribosome binding, e.g., recruitment.

In some embodiments, the eIF4G fragment is about 1,500-200 amino acids, about 1,400-300 amino acids, about 1,300-350 amino acids, about 1,200-400 amino acids, about 1,100-450 amino acids, about 1,000-500 amino acids, about 900-550 amino acids, about 800-600 amino acids, about 1,500-300 amino acids, 1,500-400 amino acids, 1,500-500 amino acids, about 1,500-600 amino acids, amino acids, about 1,500-700 amino acids, about 1,500-800 amino acids, about 1,500-900 amino acids, about 1,500-1000 amino acids, about 1,500-1,100 amino acids, about 1,500-1,200 amino acids, about 1,500-1,300 amino acids, about 1,500-1,400 amino acids, about 1,400-200 amino acids, about 1,300-200 amino acids, about 1,200-200 amino acids, about 1,100-200 amino acids, about 1,000-200 amino acids, about 900-200 amino acids, about 800-200 amino acids, about 700-200 amino acids, about 600-200 amino acids, or about 500-200 amino acids in length.

In some embodiments, the eIF4G fragment is about 500 amino acids in length. In some embodiments, the eIF4G fragment is about 600 amino acids in length. In some embodiments, the eIF4G fragment is about 700 amino acids in length. In some embodiments, the eIF4G fragment is about 800 amino acids in length. In some embodiments, the eIF4G fragment is about 900 amino acids in length. In some embodiments, the eIF4G fragment is about 1000 amino acids in length. In some embodiments, the eIF4G fragment is about 1100 amino acids in length. In some embodiments, the eIF4G fragment is about 1200 amino acids in length. In some embodiments, the eIF4G fragment is about 1300 amino acids in length. In some embodiments, the eIF4G fragment is about 1400 amino acids in length. In some embodiments, the eIF4G fragment is about 1500 amino acids in length.

In some embodiments, the effector molecule comprises a variant of eIF4G, e.g., as disclosed herein. In some embodiments, the eIF4G variant retains ribosome binding, e.g., recruitment. In some embodiments, the eIF4G variant comprises a mutation (e.g., substitution) in the eIF4G polypeptide sequence at any one, two, all or a combination of the following positions: amino acid 768, amino acid 771, or amino acid 776. In some embodiments, the eIF4G variant comprises a mutation, e.g., substitution, at position 768 of the eIF4G polypeptide sequence, e.g., a Leucine to Alanine substitution at position 768. In some embodiments, the eIF4G variant comprises a mutation, e.g., substitution, at position 771 of the eIF4G polypeptide sequence, e.g., a Leucine to Alanine substitution at position 771. In some embodiments, the eIF4G variant comprises a mutation, e.g., substitution, at position 776 of the eIF4G polypeptide sequence, e.g., a Phenylalanine to Alanine at position 776. In some embodiments, the eIF4G variant comprises a mutation, e.g., substitution, at position 768 of the eIF4G polypeptide sequence, e.g., an Alanine at position 768; and a mutation, e.g., substitution, at position 771 of the eIF4G polypeptide sequence, e.g., an Alanine at position 771. In some embodiments, the eIF4G variant comprises a mutation, e.g., substitution, at position 768 of the eIF4G polypeptide sequence, e.g., an Alanine at position 768; and a mutation, e.g., substitution, at position 776 of the eIF4G polypeptide sequence, e.g., an Alanine at position 776. In some embodiments, the eIF4G variant comprises a mutation, e.g., substitution, at position 771 of the eIF4G polypeptide sequence, e.g., an Alanine at position 771; and a mutation, e.g., substitution, at position 776 of the eIF4G polypeptide sequence, e.g., an Alanine at position 776. In some embodiments, the eIF4G variant comprises a mutation, e.g., substitution, at position 771 of the eIF4G polypeptide sequence, e.g., an Alanine at position 771; a mutation, e.g., substitution, at position 771 of the eIF4G polypeptide sequence, e.g., an Alanine at position 771; and a mutation, e.g., substitution, at position 776 of the eIF4G polypeptide sequence, e.g., an Alanine at position 776.

In some embodiments, the effector molecule is a part of the eIF3 complex, e.g., which can recruit the ribosome. In some embodiments, the eIF3 complex comprises eIF3d, eIF3c, eIF3e, or eIF3i, or a fragment thereof, or any combination thereof.

In some embodiments, the effector molecule comprises an amino acid sequence provided in Table 2, or a sequence with at least 80%, 85%, 90%, 95%, 96%, 97%, 98%, 99% or 100% identity thereof. In some embodiments, the effector molecule is encoded by a nucleotide sequence provided in Table 2, or a sequence with at least 80%, 85%, 90%, 95%, 96%, 97%, 98%, 99% or 100% identity thereof.

In some embodiments of any of the systems, LNP compositions, methods or uses disclosed herein, the effector molecule is a splicing factor, e.g., a splicing factor provided in Table 4, e.g., Rups1, Magoh, Y14, or a fragment or variant, or combination thereof.

In some embodiments of any of the systems, LNP compositions, methods or uses disclosed herein, the effector molecule is an RNA stabilizing factor, e.g., a splicing factor provided in Table 4, e.g., HuR or a fragment, or variant thereof.

In some embodiments of any of the systems, LNP compositions, methods or uses disclosed herein, the tether molecule binds to a binding element in the first polynucleotide. In some embodiments, the tether molecule binds to a sequence of the binding element or to a structure comprising the sequence of the binding element. In some embodiments, the tether molecule comprises an RNA binding protein or a fragment thereof.

In some embodiments, the tether molecule comprises a tether molecule provided in Table 1, e.g., MBP, PCP, Lambda N, Ul A or PUF, or 15.5 kd or a variant or fragment thereof.

In some embodiments, when the tether molecule is MBP (e.g., wildtype MBP, a variant or fragment thereof) the binding element is MS2 (e.g., wildtype MS2, or a variant or fragment thereof).

In some embodiments, when the tether molecule is PCP (e.g., wildtype PCP, or a variant or fragment thereof) the binding element is PP7 (e.g., wildtype PP7, or a variant or fragment thereof).

In some embodiments, when the tether molecule is Lambda N (e.g., wildtype Lambda N, or a variant or fragment thereof) the binding element is BoxB (e.g., wildtype BoxB, or a variant or fragment thereof).

In some embodiments, when the tether molecule is U1A (e.g., wildtype U1A, or a variant or fragment thereof) the binding element is U1A hairpin (e.g., wildtype U1A hairpin, or a variant or fragment thereof).

In some embodiments, when the tether molecule is 15.5 kd (e.g., wildtype 15.5 kd, or a variant or fragment thereof) the binding element is a kink-turn forming sequence (e.g., wildtype kink-turn forming sequence, or a variant or fragment thereof).

In some embodiments, when the tether molecule is PUF (e.g., wildtype PUF, or a variant or fragment thereof) the binding element is PRE (e.g., wildtype PRE, or a variant or fragment thereof).

In some embodiments, the tether molecule comprises MBP. In some embodiments, the tether molecule comprises an amino acid sequence provided in Table 2 or a sequence with at least 80%, 85%, 90%, 95%, 96%, 97%, 98%, 99% or 100% identity thereof. In some embodiments, the tether molecule comprises the tether molecule is encoded by a nucleotide sequence provided in Table 2 or a sequence with at least 80%, 85%, 90%, 95%, 96%, 97%, 98%, 99% or 100% identity thereof.

In some embodiments of any of the systems, LNP compositions, methods or uses disclosed herein, the therapeutic payload or prophylactic payload comprises an mRNA encoding: a secreted protein, a membrane-bound protein; or an intercellular protein.

In some embodiments, the therapeutic payload or prophylactic payload is chosen from a cytokine, an antibody, a vaccine (e.g., an antigen, an immunogenic epitope), a receptor, an enzyme, a hormone, a transcription factor, a ligand, a membrane transporter, a structural protein, a nuclease, or a component, variant or fragment (e.g., a biologically active fragment) thereof.

In some embodiments, the therapeutic payload or prophylactic payload comprises a cytokine, or a variant or fragment (e.g., a biologically active fragment)

In some embodiments, the therapeutic payload or prophylactic payload comprises an antibody or a variant or fragment (e.g., a biologically active fragment) thereof.

In some embodiments, the therapeutic payload or prophylactic payload comprises a vaccine (e.g., an antigen, an immunogenic epitope), or a component, variant or fragment (e.g., a biologically active fragment) thereof.

In some embodiments, the therapeutic payload or prophylactic payload comprises a protein or peptide.

In some embodiments, any of the systems, LNP compositions, methods or uses disclosed herein, results in one, two, three, four, five, six or all, or any combination thereof, of the following in a cell (e.g., in a cell contacted with the system or LNP composition):
(i) increased expression and/or level of mRNA encoding the therapeutic payload or prophylactic payload;
(ii) sustained expression and/or level of mRNA encoding the therapeutic payload or prophylactic payload;
(iii) increased expression and/or level of therapeutic payload or prophylactic payload;
(iv) sustained expression and/or level of therapeutic payload or prophylactic payload;
(v) increased stability of mRNA encoding the therapeutic payload or prophylactic payload;
(vi) increased resistance of translation of therapeutic payload or prophylactic payload to cellular environment, e.g., stress or nutrient deprivation or translation factor availability;
(vii) reduced dosing of the therapeutic payload or prophylactic payload; or
(viii) reduced toxicity, e.g., reduced modulation of a protein translated from endogenous mRNA in a cell.

In some embodiments, any of the systems, LNP compositions, methods or uses disclosed herein, results in increased expression and/or level of mRNA encoding the therapeutic payload or prophylactic payload.

In some embodiments, any of the systems, LNP compositions, methods or uses disclosed herein, results in sustained expression and/or level of mRNA encoding the therapeutic payload or prophylactic payload.

In some embodiments, any of the systems, LNP compositions, methods or uses disclosed herein, results in increased expression and/or level of therapeutic payload or prophylactic payload.

In some embodiments, any of the systems, LNP compositions, methods or uses disclosed herein, results in sustained expression and/or level of therapeutic payload or prophylactic payload.

In some embodiments, any of the systems, LNP compositions, methods or uses disclosed herein, results in increased stability of mRNA encoding the therapeutic payload or prophylactic payload. In some embodiments, any of the systems, LNP compositions, methods or uses disclosed herein, results in increased resistance of translation of therapeutic payload or prophylactic payload to cellular environment, e.g., stress or nutrient deprivation or translation factor availability.

In some embodiments, any of the systems, LNP compositions, methods or uses disclosed herein, results in reduced dosing of the therapeutic payload or prophylactic payload.

In some embodiments, any of the systems, LNP compositions, methods or uses disclosed herein, results in reduced toxicity, e.g., reduced modulation of a protein translated from endogenous mRNA in a cell.

In some embodiments, any one, or all of (i)-(vii) is compared to a cell which:
(a) has not been contacted with the system disclosed herein;
(b) has not been contacted with the LNP composition disclosed herein;
(c) has not been contacted with an LNP comprising the first polynucleotide; or
(d) has been contacted with an LNP comprising the first polynucleotide but has not been contacted with the second polynucleotide, e.g., an LNP comprising the second polynucleotide.

In some embodiments, any of the systems, LNP compositions, methods or uses disclosed herein, result in increased expression, duration of expression, and/or level of the mRNA encoding the therapeutic payload or prophylactic payload, e.g., as measured by an assay in Example 2 or 3. In some embodiments, the increased expression, duration of expression, and/or level of the mRNA encoding the therapeutic payload or prophylactic payload is compared to an otherwise similar cell that has been contacted with an mRNA encoding the therapeutic payload or prophylactic payload which mRNA lacks a binding element of the first polynucleotide. In some embodiments, the increase in expression, duration of expression and/or level of the mRNA encoding the therapeutic payload or prophylactic payload is about 1.5, 2, 3, 4, 5, 6, 7, 8, 9, 10, 20, 25, 30, 35, 40 or 50 fold.

In some embodiments, the increase in expression and/or level of the mRNA comprises an increase in stability (e.g., half-life) of the mRNA encoding the therapeutic payload or prophylactic payload, e.g., about 1.5, 2, 3, 4, 5, 6, 7, 8, 9, 10, or 20 fold increase in stability of the mRNA encoding the therapeutic payload or prophylactic payload. In some embodiments, the mRNA encoding the therapeutic payload or prophylactic payload has a half-life of about 3-25 hours, about 4-20 hours, about 4-15 hours, about 5-10 hours, about 6-9 hours or about 7-8 hours.

In some embodiments, any of the systems, LNP compositions, methods or uses disclosed herein, results in sustained expression and/or level of the mRNA encoding the therapeutic payload or prophylactic payload, e.g., as measured by an assay in Example 4. In some embodiments, at least 15%, 20%, 30%, 40%, 50%, 60%, 70%, 80%, 90% or more of the expression and/or level of the mRNA encoding the therapeutic payload or prophylactic payload is sustained for a period of time, e.g., about 1, 2, 3, 4, 5, 6, 7, 8, 9, 10, 12, 24 or 36 hours. In some embodiments, the sustained expression and/or level of the mRNA encoding the therapeutic payload or prophylactic payload is compared to an otherwise similar cell that has been contacted with an mRNA encoding the therapeutic payload or prophylactic payload, which mRNA lacks a binding element of the first polynucleotide. As used herein, the term “sustained expression” refers to a longer duration of expression and/or longer maintainence of mRNA levels compared to mRNA expression in an otherwise similar cell that has been contacted with an mRNA encoding the therapeutic payload or prophylactic payload, which mRNA lacks a binding element of the first polynucleotide.

In some embodiments, any of the systems, LNP compositions, methods or uses disclosed herein, results in a decreased loss, e.g., about a 1.2-fold, 2-fold, 3-fold, 4-fold or 5-fold decrease in loss, of mRNA encoding the therapeutic payload or prophylactic payload. In some embodiments, the decrease in loss of mRNA encoding the therapeutic payload or prophylactic payload occurs over a period of time, e.g., about 1, 2, 3, 4, 5, 6, 7, 8, 9, 10, 12, or 24 hours.

In some embodiments, any of the systems, LNP compositions, methods or uses disclosed herein, results in a decreased loss, e.g., about a 1.2-fold, 2-fold, 3-fold, 4-fold, 5-fold, 6-fold, 7-fold, 8-fold, 9-fold or 10-fold decrease in loss, of translating mRNA. In some embodiments, the decrease in loss of mRNA encoding the therapeutic payload or prophylactic payload occurs over a period of time, e.g., about 1, 2, 3, 4, 5, 6, 7, 8, 9, 10, 12, or 24 hours.

In some embodiments, any of the systems, LNP compositions, methods or uses disclosed herein, results in a sustained, e.g., maintained, level of translation of an mRNA encoding the therapeutic payload or prophylactic payload.

In some embodiments, any of the systems, LNP compositions, methods or uses disclosed herein, results in increased expression, duration of expression, and/or level of the therapeutic payload or prophylactic payload, e.g., increased protein level, translation, or half-life, e.g., as measured by an assay of Example 4. In some embodiments, the increased expression and/or level of the therapeutic payload or prophylactic payload is compared to an otherwise similar cell that has been contacted with an mRNA encoding the therapeutic payload or prophylactic payload which mRNA lacks a binding element of the first polynucleotide. In some embodiments, the increase in expression and/or level of the therapeutic payload or prophylactic payload is about 1.5, 2, 3, 4, 5, 6, 7, 8, 9, 10, 20, 25, 30, 35, 40 or 50 fold.

In some embodiments, any of the systems, LNP compositions, methods or uses disclosed herein, results in sustained expression and/or level of the therapeutic payload or prophylactic payload.

In some embodiments of any of the systems, LNP compositions, methods or uses disclosed herein, the first polynucleotide and the second polynucleotide each comprises an mRNA.

In some embodiments of any of the systems, LNP compositions, methods or uses disclosed herein, the tether molecule of (b)(2) binds to, e.g., recognizes, the binding element of (a)(2).

In some embodiments of any of the systems, LNP compositions, methods or uses disclosed herein, the first polynucleotide is formulated as an LNP or the second polynucleotide is formulated as an LNP.

In some embodiments of any of the systems, LNP compositions, methods or uses disclosed herein, the first polynucleotide is formulated as an LNP and the second polynucleotide is formulated as an LNP, e.g., the same LNP or different LNPs.

In some embodiments of any of the systems, LNP compositions, methods or uses disclosed herein, the LNP comprising the first polynucleotide and the LNP comprising the second polynucleotide is the same LNP.

In some embodiments of any of the systems, LNP compositions, methods or uses disclosed herein, the LNP comprising the first polynucleotide and the LNP comprising the second polynucleotide are different LNPs.

In some embodiments of any of the systems, LNP compositions, methods or uses disclosed herein, the binding element comprises a sequence comprising 19 nucleotides, e.g., a binding element nucleotide sequence provided in Table 2 or a sequence with at least 80%, 85%, 90%, 95%, 96%, 97%, 98%, 99% or 100% identity thereof.

In some embodiments of any of the systems, LNP compositions, methods or uses disclosed herein, the spacer sequence comprises 20 nucleotides, e.g., a spacer sequence provided in Table 2 or a sequence with at least 80%, 85%, 90%, 95%, 96%, 97%, 98%, 99% or 100% identity thereof.

In some embodiments of any of the systems, LNP compositions, methods or uses disclosed herein, the effector molecule comprises an amino acid sequence provided in Table 2, or a sequence with at least 80%, 85%, 90%, 95%, 96%, 97%, 98%, 99% or 100% identity thereof.

In some embodiments of any of the systems, LNP compositions, methods or uses disclosed herein, the effector molecule is encoded by a nucleotide sequence provided in Table 2, or a sequence with at least 80%, 85%, 90%, 95%, 96%, 97%, 98%, 99% or 100% identity thereof.

In some embodiments of any of the systems, LNP compositions, methods or uses disclosed herein, the tether molecule comprises an amino acid sequence provided in Table 2 or a sequence with at least 80%, 85%, 90%, 95%, 96%, 97%, 98%, 99% or 100% identity thereof.

In some embodiments of any of the systems, LNP compositions, methods or uses disclosed herein, the tether molecule is encoded by a nucleotide sequence provided in Table 2 or a sequence with at least 80%, 85%, 90%, 95%, 96%, 97%, 98%, 99% or 100% identity thereof.

In some embodiments of any of the systems, LNP compositions, methods or uses disclosed herein, the first polynucleotide comprises an mRNA comprising at least one chemical modification.

In some embodiments of any of the systems, LNP compositions, methods or uses disclosed herein, the LNP is formulated for intravenous, subcutaneous, intramuscular, intranasal, intraocular, rectal, pulmonary or oral delivery. In some embodiments, the LNP is formulated for intravenous delivery. In some embodiments, the LNP is formulated for subcutaneous delivery. In some embodiments, the LNP is formulated for intramuscular delivery. In some embodiments, the LNP is formulated for intranasal delivery. In some embodiments, the LNP is formulated for intraocular delivery. In some embodiments, the LNP is formulated for rectal delivery. In some embodiments, the LNP is formulated for pulmonary delivery. In some embodiments, the LNP is formulated for oral delivery.

In some embodiments of any of the systems, LNP compositions, methods or uses disclosed herein, the LNP further comprising a pharmaceutically acceptable carrier or excipient.

In some embodiments of any of the systems, LNP compositions, methods or uses disclosed herein, the polynucleotide, e.g., the first and/or second polynucleotide comprises a cap, a 3′ UTR, a 5′ UTR, a Poly A tail and/or a micro RNA (miRNA) binding site. In some embodiments, the cap comprises a cap disclosed herein. In some embodiments, the polynucleotide, e.g., the first and/or second polynucleotide does not comprise a cap. In some embodiments, the 3′ UTR comprises a 3′ UTR disclosed herein, e.g., a v1.1 3′ UTR or a sequence with at least 80%, 85%, 90%, 95%, 96%, 97%, 98%, 99%, or 100% identity thereto. In some embodiments, the 5′ UTR comprises a 5′ UTR disclosed herein. In some embodiments, the Poly A tail comprises a Poly A tail sequence disclosed herein or a fragment thereof. In some embodiments, the polynucleotide, e.g., the first and/or second polynucleotide does not comprise a Poly A tail. In some embodiments, the miRNA binding site comprises a miRNA binding site disclosed herein.

In some embodiments, the polynucleotide, e.g., the first and/or second polynucleotide is a circular polynucleotide. In some embodiments, the first polynucleotide is a circular polynucleotide. In some embodiments, the second polynucleotide is a circular polynucleotide.

In some embodiments of any of the methods or uses disclosed herein, the LNP comprising the first polynucleotide is administered at a lower dose compared to a reference LNP. In some embodiments, the LNP comprising the first polynucleotide is administered at a dose that is at least 10%, 20%, 30%, 40%, 50%, 60%, 70%, 80%, or 90% lower compared to the dose of a reference LNP. In some embodiments, the reference LNP is chosen from: an otherwise similar LNP comprising a polynucleotide which does not have the binding element of the first polynucleotide; or an LNP that does not comprise the second polynucleotide.

In some embodiments, the LNP comprising the first polynucleotide is administered at a higher dose compared to the LNP comprising the second polynucleotide. In some embodiments, the LNP comprising the first polynucleotide is administered at a dose that is at least 10%, 20%, 30%, 40%, 50%, 60%, 70%, 80%, or 90% higher compared to the dose of the LNP comprising the second polynucleotide. In some embodiments, the LNP comprising the first polynucleotide is in molar excess compared to the LNP comprising the second polynucleotide.

In some embodiments, the LNP comprising the first polynucleotide is in about 1-800× molar excess, compared to the LNP comprising the second polynucleotide. In some embodiments, the LNP comprising the first polynucleotide is in about 1-750×, about 2-700×, about 3-650×, about 4-600×, about 5-550×, about 6-500×, about 7-450 x, about 8-400×, about 10-350×, about 15-300×, about 20-250×, about 25-200×, about 30-150×, about 35-100×, about 40-90×, about 45-80×, about 50-75×, about 60-70× molar excess compared to the LNP comprising the second polynucleotide. In some embodiments, the LNP comprising the first polynucleotide is in about 2×, about 3×, about 4×, about 5×, about 6×, about 7×, about 8×, about 9×, about 10×, about 11×, about 12×, about 13×, about 14×, about 15×, about 20×, about 25×, about 30×, about 35×, about 40×, about 50×, about 60×, about 70×, about 80×, about 90×, about 100 x, about 150×, about 200×, about 250×, about 300×, about 350×, about 400×, about 450×, about 500×, about 600×, about 650×, about 700×, about 750×, or about 800× molar excess compared to the LNP comprising the second polynucleotide.

In some embodiments, the LNP comprising the first polynucleotide is in about 9× molar excess compared to the LNP comprising the second polynucleotide.

In some embodiments, the LNP comprising the first polynucleotide is in about 10× molar excess compared to the LNP comprising the second polynucleotide.

In some embodiments, the LNP comprising the first polynucleotide and the LNP comprising the second polynucleotide are at the same molar amounts.In an embodiment of any of the LNP compositions, systems, methods or compositions for use disclosed herein, the first and second polynucleotides are formulated at an (a):(b) mass ratio of 10:1, 8:1, 6:1, 4:1, 3:1, 2:1, 1.5:1, or 1:1. In an embodiment, the first and second polynucleotides are formulated at an (a):(b) mass ratio of 1:1, 1.1.5, 1:2, 1:3, 1:4, 1:6, 1:8, or 1:10. In an embodiment, the first and second polynucleotides are formulated at an (a):(b) mass ratio of 1:1.

In an embodiment of any of the LNP compositions, systems, methods or compositions for use disclosed herein, the first polynucleotide, the second polynucleotide, or both, comprises at least one chemical modification. In an embodiment, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In an embodiment, the chemical modification is selected from the group consisting of pseudouridine, N1-methylpseudouridine, 5-methylcytosine, 5-methoxyuridine, and a combination thereof. In an embodiment, the chemical modification is N1-methylpseudouridine. In an embodiment, each mRNA in the lipid nanoparticle comprises fully modified N1-methylpseudouridine. In some embodiments, the first polynucleotide, the second polynucleotide, or both, do not comprise any chemical modification.

In an embodiment of any of the LNP compositions, systems, methods or compositions for use disclosed herein, the LNP composition comprises: (i) an ionizable lipid, e.g., an amino lipid; (ii) a sterol or other structural lipid; (iii) a non-cationic helper lipid or phospholipid; and, optionally, (iv) a PEG-lipid.

In an embodiment of any of the LNP compositions, systems, methods or compositions for use disclosed herein, the LNP composition comprises an ionizable lipid comprising an amino lipid. In an embodiment, the ionizable lipid comprises a compound of any of Formulae (I), (IA), (IB), (II), (IIa), (IIb), (IIc), (IId), (IIe), (IIf), (IIg), (III), (IIIa1), (IIIa2), (IIIa3), (IIIa4), (IIIa5), (IIIa6), (IIIa7), or (IIIa8). In an embodiment, the ionizable lipid comprises a compound of Formula (I). In an embodiment, the ionizable lipid comprises a compound of Formula (IIa). In an embodiment, the ionizable lipid comprises a compound of Formula (IIe).

In an embodiment of any of the LNP compositions, systems, methods or compositions for use disclosed herein, the LNP composition comprises a non-cationic helper lipid or phospholipid comprising a compound selected from the group consisting of 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In an embodiment, the phospholipid is DSPC, e.g., a variant of DSPC, e.g., a compound of Formula (IV).

In an embodiment of any of the LNP compositions, systems, methods or compositions for use disclosed herein, the LNP composition comprises a structural lipid. In one embodiment, the structural lipid is a phytosterol or a combination of a phytosterol and cholesterol. In one embodiment, the phytosterol is selected from the group consisting of β-sitosterol, stigmasterol, β-sitostanol, campesterol, brassicasterol, and combinations thereof.

In one embodiment, the structural lipid can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In one embodiment, the structural lipid is selected from selected from β-sitosterol and cholesterol. In an embodiment, the structural lipid is β-sitosterol. In an embodiment, the structural lipid is cholesterol.

In an embodiment of any of the LNP compositions, systems, methods or compositions for use disclosed herein, the LNP composition comprises a PEG lipid. In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In an embodiment, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In an embodiment, the PEG lipid is selected from the group consisting of PEG-c-DOMG, PEG-DMG, PEG-DLPE, PEG-DMPE, PEG-DPPC and PEG-DSPE lipid. In an embodiment, the PEG-lipid is PEG-DMG.

In an embodiment, the PEG lipid is chosen from a compound of: Formula (V), Formula (VI-A), Formula (VI-B), Formula (VI-C) or Formula (VI-D). In an embodiment, the PEG-lipid is a compound of Formula (VI-A). In an embodiment, the PEG-lipid is a compound of Formula (VI-B). In an embodiment, the PEG-lipid is a compound of Formula (VI-C). In an embodiment, the PEG-lipid is a compound of Formula (VI-D).

In an embodiment of any of the LNP compositions, systems, methods or compositions for use disclosed herein, the LNP comprises about 20 mol % to about 60 mol % ionizable lipid, about 5 mol % to about 25 mol % non-cationic helper lipid or phospholipid, about 25 mol % to about 55 mol % sterol or other structural lipid, and about 0.5 mol % to about 15 mol % PEG lipid. In one embodiment of the LNPs or methods of the disclosure, the LNP comprises about 35 mol % to about 55 mol % ionizable lipid, about 5 mol % to about 25 mol % non-cationic helper lipid or phospholipid, about 30 mol % to about 40 mol % sterol or other structural lipid, and about 0 mol % to about 10 mol % PEG lipid. In one embodiment of the LNPs or methods of the disclosure, the LNP comprises about 50 mol % ionizable lipid, about 10 mol % non-cationic helper lipid or phospholipid, about 38.5 mol % sterol or other structural lipid, and about 1.5 mol % PEG lipid. In one embodiment of the LNPs or methods of the disclosure, the LNP comprises about 49.83 mol % ionizable lipid, about 9.83 mol % non-cationic helper lipid or phospholipid, about 30.33 mol % sterol or other structural lipid, and about 2.0 mol % PEG lipid.

In one embodiment, the mol % sterol or other structural lipid is 18.5% phytosterol and the total mol % structural lipid is 38.5%. In one embodiment, the mol % sterol or other structural lipid is 28.5% phytosterol and the total mol % structural lipid is 38.5%.

In one embodiment of the LNPs, systems or methods of the disclosure, the LNP comprises about 50 mol % a compound of Formula (IIa) and about 10 mol % non-cationic helper lipid or phospholipid. In one embodiment of the LNPs or methods of the disclosure, the LNP comprises 50 mol % a compound of Formula (IIa) and about 10 mol % non-cationic helper lipid or phospholipid. In one embodiment of the LNPs, systems, or methods of the disclosure, the LNP comprises about 50 mol % a compound of Formula (IIa) and 10 mol % non-cationic helper lipid or phospholipid. In one embodiment of the LNPs or methods of the disclosure, the LNP comprises 50 mol % a compound of Formula (IIa) and 10 mol % non-cationic helper lipid or phospholipid. In one embodiment of the LNPs or methods of the disclosure, the LNP comprises about 49.83 mol % a compound of Formula (IIa), about 9.83 mol % non-cationic helper lipid or phospholipid, about 30.33 mol % sterol or other structural lipid, and about 2.0 mol % PEG lipid.

In one embodiment of the LNPs, systems, or methods of the disclosure, the LNP comprises about 50 mol % a compound of Formula (IIe) and about 10 mol % non-cationic helper lipid or phospholipid. In one embodiment of the LNPs or methods of the disclosure, the LNP comprises 50 mol % a compound of Formula (IIe) and about 10 mol % non-cationic helper lipid or phospholipid. In one embodiment of the LNPs or methods of the disclosure, the LNP comprises about 50 mol % a compound of Formula (IIe) and 10 mol % non-cationic helper lipid or phospholipid. In one embodiment of the LNPs or methods of the disclosure, the LNP comprises 50 mol % a compound of Formula (IIe) and 10 mol % non-cationic helper lipid or phospholipid. In one embodiment of the LNPs or methods of the disclosure, the LNP comprises about 49.83 mol % a compound of Formula (IIe), about 9.83 mol % non-cationic helper lipid or phospholipid, about 30.33 mol % sterol or other structural lipid, and about 2.0 mol % PEG lipid.

In an embodiment of any of the LNP compositions, systems, methods or compositions for use disclosed herein, the LNP is formulated for intravenous, subcutaneous, intramuscular, intraocular, intranasal, rectal, pulmonary or oral delivery. In an embodiment, the LNP is formulated for intravenous delivery. In an embodiment, the LNP is formulated for subcutaneous delivery. In an embodiment, the LNP is formulated for intramuscular delivery. In an embodiment, the LNP is formulated for intraocular delivery. In an embodiment, the LNP is formulated for intranasal delivery. In an embodiment, the LNP is formulated for rectal delivery. In an embodiment, the LNP is formulated for pulmonary delivery. In an embodiment, the LNP is formulated for oral delivery.

In an embodiment of any of the methods or compositions for use disclosed herein, the subject is a mammal, e.g., a human.

Additional features of any of the aforesaid LNP compositions or methods of using said LNP compositions, include one or more of the following enumerated embodiments. Those skilled in the art will recognize or be able to ascertain using no more than routine experimentation, many equivalents to the specific embodiments of the invention described herein. Such equivalents are intended to be encompassed by the following enumerated embodiments.

### Other Embodiments of the Disclosure

E1. A lipid nanoparticle (LNP) composition comprising:

(a) a first polynucleotide comprising: (1) a sequence encoding a therapeutic payload or prophylactic payload, and (2) a binding element; and

(b) a second polynucleotide comprising a sequence encoding: (1) an effector molecule, and/or (2) a polypeptide that recognizes the binding element (a tether molecule),

optionally wherein, (a) and (b) each comprise an mRNA.
E2. The LNP composition of embodiment E1, wherein the first polynucleotide and the second polynucleotide are disposed in the same polynucleotide.
E3. The LNP composition of embodiment E1, wherein the first polynucleotide and the second polynucleotide are separated by a protease cleavage site (e.g., a P2A, T2A, E2A, or TPE (P2A-T2A-E2A) site) or an internal ribosomal entry site.
E4. The LNP composition of embodiment E1, wherein the first polynucleotide and the second polynucleotide are disposed in different polynucleotides.
E5. The LNP composition of embodiment E1, wherein (a) and (b) each are formulated as LNPs, e.g., the same LNP.
E6. The LNP composition of embodiment E1, wherein (a) and (b) are formulated as different LNPs, optionally wherein:

(i) (a) formulated as an LNP is in a first composition and (b) formulated as an LNP is in a second composition, e.g., (b) and (b) are in different compositions; or

(ii) (a) formulated as an LNP and (b) formulated as an LNP are in the same composition.
E7. A system comprising:

(a) a first polynucleotide comprising: (1) a sequence encoding a therapeutic payload or prophylactic payload, and (2) a binding element; and/or

(b) a second polynucleotide comprising a sequence encoding: (1) an effector molecule, and/or a (2) polypeptide that recognizes the binding element (a tether molecule),

optionally wherein, (a) and (b) each comprise an mRNA.
E8. The system of embodiment E7, wherein the system comprises (a).
E9. The system of embodiment E7, wherein the system comprises (b).
E10. The system of embodiment E7, wherein the system comprises (a) and (b).
E11. The system of any one of embodiments E7-E10, wherein the first polynucleotide and the second polynucleotide are disposed in the same polynucleotide.
E12. The system of embodiment Ell, wherein the first polynucleotide and the second polynucleotide are separated by a protease cleavage site (e.g., a P2A, T2A, E2A, or TPE (P2A-T2A-E2A) site) or an internal ribosomal entry site.
E13. The system of any one of embodiments E7-E10, wherein the first polynucleotide and the second polynucleotide are disposed in different polynucleotides.
E14. The system of any one of embodiments E7-E13, wherein the therapeutic payload or prophylactic payload is not a reporter protein.
E15. The system of any one of embodiments E7-E13, wherein the system comprises less than 5%, 10%, 15%, 20%, 25%, or 50% of a cellular impurity, e.g., a cellular component, e.g., a membrane, protein or lipid from a cell.
E16. The system of any one of embodiments E7-E15, wherein at least one of (a) or (b) is formulated as a lipid nanoparticle (LNP).
E17. The system, or LNP composition of embodiment E16, wherein (a) is formulated as an LNP.
E18. The system, or LNP composition of embodiment E16, wherein (b) is formulated as an LNP.
E19. The system, or LNP composition of embodiment E16, wherein (a) and (b) both are formulated as LNPs, e.g., the same LNP or different LNPs, optionally wherein:

(i) (a) formulated as an LNP is in a first composition and (b) formulated as an LNP is in a second composition, e.g., (b) and (b) are in different compositions; or

(ii) (a) formulated as an LNP and (b) formulated as an LNP are in the same composition.
E20. The method or system, or LNP composition of any one of the preceding embodiments, wherein the tether molecule of the second polynucleotide comprises an RNA binding protein or a fragment thereof which binds to, e.g., recognizes, the binding element of the first polynucleotide.
E21. The method, or system, or LNP composition of any one of the preceding embodiments, wherein the binding element of the first polynucleotide is situated upstream (5′) or downstream (3′) or in the open reading frame of the sequence encoding the therapeutic payload or prophylactic payload.
E22. The method, or system, or LNP composition of any one of the preceding embodiments, wherein the binding element of the first polynucleotide is situated upstream (5′) or downstream (3′) of a 5′ UTR of the first polynucleotide.
E23. The system, or LNP composition of any one of the preceding embodiments, wherein the binding element of the first polynucleotide is situated upstream (5′) or downstream (3′) of a 3′ UTR of the first polynucleotide.
E24. The system, or LNP composition of any one of the preceding embodiments, wherein the binding element of the first polynucleotide is situated downstream of a 3′ UTR of the first polynucleotide.
E25. The system, or LNP composition of any one of the preceding embodiments, wherein the binding element of the first polynucleotide is bound by the tether molecule of the second polynucleotide, e.g., a tether molecule provided in Table 1, e.g., MBP, PCP, Lambda N, U1A or PUF, or 15.5 kd or a variant or fragment thereof.
E26. The system, or LNP composition of any one of the preceding embodiments, wherein the binding element comprises a sequence which is bound, e.g., recognized, by the tether molecule.
E27. The system, or LNP composition of any one of the preceding embodiments, wherein the binding element comprises a sequence comprising a structure that is bound, e.g., recognized, by the tether molecule.
E28. The system, or LNP composition of any one of the preceding embodiments, wherein the binding element is chosen from a binding element provided in Table 1, e.g., MS2, PP7, BoxB, U1A hairpin or PRE or kink-turn, or a variant or fragment thereof.
E29. The system, or LNP composition of any one of the preceding embodiments, wherein when the binding element is MS2 (e.g., wildtype MS2, or a variant or fragment thereof) the tether molecule is MBP (e.g., wildtype MBP, a variant or fragment thereof).
E30. The system, or LNP composition of any one of the preceding embodiments, wherein when the binding element is PP7 (e.g., wildtype PP7, or a variant or fragment thereof) the tether molecule is PCP (e.g., wildtype PCP, or a variant or fragment thereof).
E31 The system, or LNP composition of any one of the preceding embodiments, wherein when the binding element is BoxB (e.g., wildtype BoxB, or a variant or fragment thereof) the tether molecule is Lambda N (e.g., wildtype Lambda N, or a variant or fragment thereof).
E32. The system, or LNP composition of any one of the preceding embodiments, wherein when the binding element is U1A hairpin (e.g., wildtype U1A hairpin, or a variant or fragment thereof) the tether molecule is U1A (e.g., wildtype U1A, or a variant or fragment thereof).
E33. The system, or LNP composition of any one of the preceding embodiments, wherein when the binding element is PRE (e.g., wildtype PRE, or a variant or fragment thereof) the tether molecule is PUF (e.g., wildtype PUF, or a variant or fragment thereof).
E34. The system, or LNP composition of any one of the preceding embodiments, wherein when the binding element is a kink-turn forming sequence and the tether molecule is 15.5 kd (e.g., wildtype 15.5 kd, or a variant or fragment thereof).
E35. The system, or LNP composition of any one of the preceding embodiments, wherein the binding element comprises a sequence comprising 5, 6, 7, 8, 9, 10, 11, 12, 13, 14, 15, 16, 17, 18, 19, 20, 21, 22, 23, 24, 25, 30, 40, 50, 60, 70, 80, 90 or 100 nucleotides.
E36. The system, or LNP composition of any one of the preceding embodiments, wherein the binding element comprises a sequence comprising about 5-100, about 5-90, about 5-80, about 5-70, about 5-60, about 5-50, about 5-40, about 5-30, about 5-25, about 5-20, ab out 5-19, about 5-18, about 5-17, about 5-16, about 5-15, about 5-14, about 5-13, about 5-12, about 5-11, about 5-10, about 5-9, about 5-8, about 5-7 or about 5-6 nucleotides.
E37. The system, or LNP composition of any one of the preceding embodiments, wherein the binding element comprises a sequence comprising about 5-100, about 6-100, about 7-100, about 8-100, about 9-100, about 10-100, about 11-100, about 12-100, about 13-100, about 14-100, about 15-100, about 16-100, about 17-100, about 18-100, about 19-100, about 20-100, about 21-100, about 22-100, about 23-100, about 24-100, about 25-100, about 30-100, about 40-100, about 50-100, about 60-100, about 70-100, about 80-100, or about 90-100 nucleotides.
E38. The system, or LNP composition of any one of the preceding embodiments, wherein the binding element comprises a sequence comprising about 5-100, about 6-90, about 7-80, about 8-70, about 9-60, about 10-50, about 11-40, about 12-30, about 13-25, about 14-24, about 15-23, about 16-22, about 17-21, or about 18-20 nucleotides.
E39. The system, or LNP composition of any one of the preceding embodiments, wherein the binding element comprises a sequence comprising 19 nucleotides, e.g., a binding element nucleotide sequence provided in Table 2 or a sequence with at least 80%, 85%, 90%, 95%, 96%, 97%, 98%, 99% or 100% identity thereof.
E40. The system, or LNP composition of any one of the preceding embodiments, wherein the binding element comprises at least 1, 2, 3, 4, 5, 6, 7, 8, 9, 10, 11, 12, 13, 14, 15, 20 or 30 repeats of the sequence bound by the tether molecule of the second polynucleotide.
E41. The system, or LNP composition of any one of the preceding embodiments, wherein the binding element comprises no more than 80, 70, 60, 50, 40 or 30 repeats of the sequence bound by the tether molecule of the second polynucleotide.
E42. The system, or LNP composition of any one of the preceding embodiments, wherein the binding element comprises about 1-30, about 1-20, about 1-10, about 1-9, about 1-8, about 1-7, about 1-6, about 1-5, about 1-4, about 1-3, or about 1-2 repeats of the sequence bound by the tether molecule of the second polynucleotide.
E43. The system, or LNP composition of any one of the preceding embodiments, wherein the binding element comprises about 1-30, about 2-30, about 3-30, about 4-30 about, 5-30 about, 6-30, about 7-30, about 8-30, about 9-30, about 10-30, about 11-30, about 12-30, about 13-30, about 14-30, about 15-30, or about 20-30 repeats of the sequence bound by the tether molecule of the second polynucleotide.
E44. The system, or LNP composition of any one of the preceding embodiments, wherein the binding element comprises about 1-30, about 2-20, about 3-15, about 4-14, about 5-13, about 6-12, about 7-11, or about 8-10 repeats of the sequence bound by the tether molecule of the second polynucleotide.
E45. The system, or LNP composition of any one of the preceding embodiments, wherein the binding element comprises 6 repeats of the sequence bound by the tether molecule of the second polynucleotide.
E46. The system, or LNP composition of any one of embodiments 40-45, wherein each repeat is separated by a spacer sequence comprising 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90 or 100 nucleotides.
E47. The system, or LNP composition of embodiment 46, wherein the spacer sequence comprises about 1-100, about 1-90, about 1-80, about 1-70, about 1-60, about 1-50, about 1-40, about 1-30, about 1-25, about 1-20, about 1-19, about 1-18, about 1-17, about 1-16, about 1-15, about 1-14, about 1-13, about 1-12, about 1-11, about 1-10, about 1-9, about 1-8, about 1-7, about 1-6, about 1-5, about 1-4, about 1-3, or about 1-2 nucleotides.
E48. The system, or LNP composition of embodiment 46 or 47, wherein the spacer sequence comprises about 1-100, about 2-100, about 3-100, about 4-100, about 5-100, about 6-100, about 7-100, about 8-100, about 9-100, about 10-100, about 11-100, about 12-100, about 13-100, about 14-100, about 15-100, about 16-100, about 17-100, about 18-100, about 19-100, about 20-100, about 21-100, about 22-100, about 23-100, about 24-100, about 25-100, about 30-100, about 40-100, about 50-100, about 60-100, about 70-100, about 80-100, or about 90-100 nucleotides.
E49. The system, or LNP composition of any one of embodiments 46-48, wherein the spacer sequence comprises about 1-100, about 2-90, about 3-80, about 4-70, about 5-60, about 6-50, about 7-40, about 8-40, about 9-30, about 10-25, about 11-24, about 12-23, about 13-22, about 14-21, about 15-20, about 16-19, about 17-18 nucleotides.
E50. The system, or LNP composition of any one of embodiments 46-49, wherein the spacer sequence comprises 20 nucleotides, e.g., a spacer sequence provided in Table 2 or a sequence with at least 80%, 85%, 90%, 95%, 96%, 97%, 98%, 99% or 100% identity thereof.
E51. The system, or LNP composition of any one of the preceding embodiments, wherein the effector molecule is chosen from: a translation factor, a splicing factor, an RNA stabilizing factor, an RNA editing factor, an RNA-binding factor, an RNA localizing factor, or a combination thereof.
E52. The system, or LNP composition of any one of the preceding embodiments, wherein the effector molecule is a translation factor, e.g., a translation factor provided in Table 4, e.g., eIF4G; Poly A binding protein (PABP); eIF3d or a component thereof; Daz1, or a fragment, or variant or combination thereof.
E53. The system, or LNP composition of any one of the preceding embodiments, wherein the effector molecule is a translation factor which modulates, e.g., facilitates, ribosome binding, e.g., recruitment, pre-initiation complex formation, or RNA unwinding.
E54. The system, or LNP composition of any one of the preceding embodiments, wherein the effector molecule comprises eIF4G, e.g., wildtype eIF4G, a variant of eIF4G, or a fragment thereof.
E55. The system, or LNP composition of any one of the preceding embodiments, wherein the effector molecule comprises a fragment of eIF4G, e.g., as disclosed herein.
E56. The system, or LNP composition of any one of the preceding embodiments, wherein the eIF4G fragment retains ribosome binding, e.g., recruitment.
E57. The system, or LNP composition of any one of embodiments 54-56, wherein the eIF4G fragment is about 1,200-200 amino acids, about 1,100-300 amino acids, 1,000-400 amino acids, 900-450 amino acids, 800-500 amino acids, 700-550 amino acids, 600-650 amino acids, 1,200-300 amino acids, 1,200-400 amino acids, 1,200-500 amino acids, 1,200-600 amino acids, 1,100-200 amino acids, 1,000-200 amino acids, 900-200 amino acids, 800-200 amino acids, 700-200 amino acids, 600-200 amino acids, or 500-200 amino acids, amino acids in length.
E58. The system, or LNP composition of any one of embodiments 54-57, wherein the eIF4G fragment is about 500 amino acids in length.
E59. The system, or LNP composition of any one of embodiments 54-57, wherein the eIF4G fragment is about 1,100 amino acids in length.
E60. The system, or LNP composition of any one of the preceding embodiments, wherein the effector molecule comprises a variant of eIF4G, e.g., as disclosed herein.
E61. The system, or LNP composition of any one of embodiments 54-60, wherein the eIF4G variant retains ribosome binding, e.g., recruitment.
E62. The system, or LNP composition of any one of embodiments 54-61, wherein the eIF4G variant comprises a mutation (e.g., substitution) in the eIF4G polypeptide sequence at any one, two, all or a combination of the following positions: amino acid 768, amino acid 771, or amino acid 776.
E63. The system, or LNP composition of embodiment 62, wherein the eIF4G variant comprises a mutation, e.g., substitution, at position 768 of the eIF4G polypeptide sequence, e.g., a Leucine to Alanine substitution at position 768.
E64. The system, or LNP composition of embodiment 62, wherein the eIF4G variant comprises a mutation, e.g., substitution, at position 771 of the eIF4G polypeptide sequence, e.g., a Leucine to Alanine substitution at position 771.
E65. The system, or LNP composition of embodiment 62, wherein the eIF4G variant comprises a mutation, e.g., substitution, at position 776 of the eIF4G polypeptide sequence, e.g., a Phenylalanine to Alanine at position 776.
E66. The system, or LNP composition of embodiment 62, wherein the eIF4G variant comprises a mutation, e.g., substitution, at position 768 of the eIF4G polypeptide sequence, e.g., an Alanine at position 768; and a mutation, e.g., substitution, at position 771 of the eIF4G polypeptide sequence, e.g., an Alanine at position 771.
E67. The system, or LNP composition of embodiment 62, wherein the eIF4G variant comprises a mutation, e.g., substitution, at position 768 of the eIF4G polypeptide sequence, e.g., an Alanine at position 768; and a mutation, e.g., substitution, at position 776 of the eIF4G polypeptide sequence, e.g., an Alanine at position 776.
E68. The system, or LNP composition of embodiment 62, wherein the eIF4G variant comprises a mutation, e.g., substitution, at position 771 of the eIF4G polypeptide sequence, e.g., an Alanine at position 771; and a mutation, e.g., substitution, at position 776 of the eIF4G polypeptide sequence, e.g., an Alanine at position 776.
E69. The system, or LNP composition of embodiment 62, wherein the eIF4G variant comprises a mutation, e.g., substitution, at position 771 of the eIF4G polypeptide sequence, e.g., an Alanine at position 771; a mutation, e.g., substitution, at position 771 of the eIF4G polypeptide sequence, e.g., an Alanine at position 771; and a mutation, e.g., substitution, at position 776 of the eIF4G polypeptide sequence, e.g., an Alanine at position 776.
E70. The system, or LNP composition of any one of embodiments 54-69, wherein the effector molecule is a part of the eIF3 complex, e.g., which can recruit the ribosome.
E71. The system, or LNP composition of embodiment 70, wherein the eIF3 complex comprises eIF3d, eIF3c, eIF3e, or eIF3i, or a fragment thereof, or any combination thereof.
E72. The system, or LNP composition of any one of embodiments 54-71, wherein the effector molecule comprises an amino acid sequence provided in Table 2, or a sequence with at least 80%, 85%, 90%, 95%, 96%, 97%, 98%, 99% or 100% identity thereof.
E73. The system, or LNP composition of any one of embodiments 54-71, wherein the effector molecule is encoded by a nucleotide sequence provided in Table 2, or a sequence with at least 80%, 85%, 90%, 95%, 96%, 97%, 98%, 99% or 100% identity thereof.
E74. The system, or LNP composition of any one of embodiments 1-51, wherein the effector molecule is a splicing factor, e.g., a splicing factor provided in Table 4, e.g., Rups1, Magoh, Y14, or a fragment or variant, or combination thereof.
E75. The system, or LNP composition of any one of embodiments 1-51, wherein the effector molecule is an RNA stabilizing factor, e.g., a splicing factor provided in Table 4, e.g., HuR or a fragment, or variant thereof.
E76. The system, or LNP composition of any one of the preceding embodiments, wherein the tether molecule binds to a binding element in the first polynucleotide.
E77. The system, or LNP composition of any one of the preceding embodiments, wherein the tether molecule binds to a sequence of the binding element or to a structure comprising the sequence of the binding element.
E78. The system, or LNP composition of any one of the preceding embodiments, wherein the tether molecule comprises an RNA binding protein or a fragment thereof.
E79. The system, or LNP composition of any one of the preceding embodiments, wherein the tether molecule comprises a tether molecule provided in Table 1, e.g., MBP, PCP, Lambda N, Ul A or PUF, or 15.5 kd or a variant or fragment thereof.
E80. The system, or LNP composition of any one of the preceding embodiments, wherein when the tether molecule is MBP (e.g., wildtype MBP, a variant or fragment thereof) the binding element is MS2 (e.g., wildtype MS2, or a variant or fragment thereof).
E81. The system, or LNP composition of any one of the preceding embodiments, wherein when the tether molecule is PCP (e.g., wildtype PCP, or a variant or fragment thereof) the binding element is PP7 (e.g., wildtype PP7, or a variant or fragment thereof).
E82. The system, or LNP composition of any one of the preceding embodiments, wherein when the tether molecule is Lambda N (e.g., wildtype Lambda N, or a variant or fragment thereof) the binding element is BoxB (e.g., wildtype BoxB, or a variant or fragment thereof).
E83. The system, or LNP composition of any one of the preceding embodiments, wherein when the tether molecule is U1A (e.g., wildtype U1A, or a variant or fragment thereof) the binding element is U1A hairpin (e.g., wildtype U1A hairpin, or a variant or fragment thereof).
E84. The system, or LNP composition of any one of the preceding embodiments, wherein when the tether molecule is 15.5 kd (e.g., wildtype 15.5 kd, or a variant or fragment thereof) the binding element is a kink-turn forming sequence (e.g., wildtype kink-turn forming sequence, or a variant or fragment thereof).
E85. The system, or LNP composition of any one of the preceding embodiments, wherein when the tether molecule is PUF (e.g., wildtype PUF, or a variant or fragment thereof) the binding element is PRE (e.g., wildtype PRE, or a variant or fragment thereof).
E86. The system, or LNP composition of any one of the preceding embodiments, wherein the tether molecule comprises an amino acid sequence provided in Table 2 or a sequence with at least 80%, 85%, 90%, 95%, 96%, 97%, 98%, 99% or 100% identity thereof.
E87. The system, or LNP composition of any one of the preceding embodiments, wherein the tether molecule is encoded by a nucleotide sequence provided in Table 2 or a sequence with at least 80%, 85%, 90%, 95%, 96%, 97%, 98%, 99% or 100% identity thereof.
E88. The system, or LNP composition of any one of the preceding embodiments, wherein the therapeutic payload or prophylactic payload comprises an mRNA encoding: a secreted protein, a membrane-bound protein; or an intercellular protein.
E89. The system, or LNP composition of embodiment 88, wherein the therapeutic payload or prophylactic payload is chosen from a cytokine, an antibody, a vaccine (e.g., an antigen, an immunogenic epitope), a receptor, an enzyme, a hormone, a transcription factor, a ligand, a membrane transporter, a structural protein, a nuclease, or a component, variant or fragment (e.g., a biologically active fragment) thereof.
E90. The system, or LNP composition of embodiment 88, wherein the therapeutic payload or prophylactic payload comprises a cytokine, or a variant or fragment (e.g., a biologically active fragment) thereof.
E91. The system, or LNP composition of embodiment 88, wherein the therapeutic payload or prophylactic payload comprises an antibody or a variant or fragment (e.g., a biologically active fragment) thereof.
E92. The system, or LNP composition of embodiment 88, wherein the therapeutic payload or prophylactic payload comprises a vaccine (e.g., an antigen, an immunogenic epitope), or a component, variant or fragment (e.g., a biologically active fragment) thereof.
E93. The system, or LNP composition of any one of the preceding embodiments, wherein the therapeutic payload or prophylactic payload comprises a protein or peptide.
E94. The system, or LNP composition of any one of the preceding embodiments, which results in one, two, three, four, five, six or all, or any combination thereof, of the following in a cell (e.g., in a cell contacted with the system or LNP composition):

(i) increased expression and/or level of mRNA encoding the therapeutic payload or prophylactic payload;

(ii) sustained expression and/or level of mRNA encoding the therapeutic payload or prophylactic payload;

(iii) increased expression and/or level of therapeutic payload or prophylactic payload;

(iv) sustained expression and/or level of therapeutic payload or prophylactic payload;

(v) increased stability of mRNA encoding the therapeutic payload or prophylactic payload;

(vi) increased resistance of translation of therapeutic payload or prophylactic payload to cellular environment, e.g., stress or nutrient deprivation or translation factor availability;

(vii) reduced dosing of the therapeutic payload or prophylactic payload; or

(viii) reduced toxicity, e.g., reduced modulation of a protein translated from endogenous mRNA in a cell.
E95. The system, or LNP composition of embodiment 94, wherein any one, or all of (i)-(vii) is compared to a cell which:

(a) has not been contacted with the system of embodiment 7;

(b) has not been contacted with the LNP composition of embodiment 1;

(c) has not been contacted with an LNP comprising the first polynucleotide; or

(d) has been contacted with an LNP comprising the first polynucleotide but has not been contacted with the second polynucleotide, e.g., an LNP comprising the second polynucleotide.
E96. The system, or LNP composition of embodiment 94 or 95, wherein the system, or LNP composition results in increased expression and/or level of the mRNA encoding the therapeutic payload or prophylactic payload, e.g., as measured by an assay in Example 2 or 3.
E97. The system, or LNP composition of embodiment 96, wherein the increased expression and/or level of the mRNA encoding the therapeutic payload or prophylactic payload is compared to an otherwise similar cell that has been contacted with an mRNA encoding the therapeutic payload or prophylactic payload which mRNA lacks a binding element of the first polynucleotide.
E98. The system, or LNP composition of embodiment 96 or 97, wherein the increase in expression and/or level of the mRNA encoding the therapeutic payload or prophylactic payload is about 1.5, 2, 3, 4, 5, 6, 7, 8, 9, 10, 20, 25, 30, 35, 40 or 50 fold.
E99. The system, or LNP composition of embodiment 96 or 97, wherein the increase in expression and/or level of the mRNA comprises an increase in stability (e.g., half-life) of the mRNA encoding the therapeutic payload or prophylactic payload, e.g., about 1.5, 2, 3, 4, 5, 6, 7, 8, 9, 10, or 20 fold increase in stability of the mRNA encoding the therapeutic payload or prophylactic payload.
E100. The system, or LNP composition of embodiment 99, wherein the mRNA encoding the therapeutic payload or prophylactic payload has a half-life of about 3-25 hours, about 4-20 hours, about 4-15 hours, about 5-10 hours, about 6-9 hours or about 7-8 hours.
E101. The system, or LNP composition of embodiment 94 or 95, wherein the system or LNP composition results in sustained expression and/or level of the mRNA encoding the therapeutic payload or prophylactic payload, e.g., as measured by an assay in Example 4.
E102. The system, or LNP composition of embodiment 101, wherein at least 15%, 20%, 30%, 40%, 50%, 60%, 70%, 80%, 90% or more of the expression and/or level of the mRNA encoding the therapeutic payload or prophylactic payload is sustained for a period of time, e.g., about 1, 2, 3, 4, 5, 6, 7, 8, 9, 10, 12, 24 or 36 hours.
E103. The system, or LNP composition of embodiment 102, wherein the sustained expression and/or level of the mRNA encoding the therapeutic payload or prophylactic payload is compared to an otherwise similar cell that has been contacted with an mRNA encoding the therapeutic payload or prophylactic payload which mRNA lacks a binding element of the first polynucleotide.
E104. The system, or LNP composition of embodiment 101, wherein the system, or LNP composition results in a decreased loss, e.g., about a 1.2-fold, 2-fold, 3-fold, 4-fold or 5-fold decrease in loss, of mRNA encoding the therapeutic payload or prophylactic payload.
E105. The system, of LNP composition of embodiments 104, wherein the decrease in loss of mRNA encoding the therapeutic payload or prophylactic payload occurs over a period of time, e.g., about 1, 2, 3, 4, 5, 6, 7, 8, 9, 10, 12, or 24 hours.
E106. The system, or LNP composition of embodiment 101, wherein the system, or LNP composition results in a decreased loss, e.g., about a 1.2-fold, 2-fold, 3-fold, 4-fold, 5-fold, 6-fold, 7-fold, 8-fold, 9-fold or 10-fold decrease in loss, of translating mRNA.
E107. The system, or LNP composition of embodiment 106, wherein the decrease in loss of mRNA encoding the therapeutic payload or prophylactic payload occurs over a period of time, e.g., about 1, 2, 3, 4, 5, 6, 7, 8, 9, 10, 12, or 24 hours.
E108. The system, or LNP composition of embodiment 101, wherein the system, or LNP composition results in a sustained, e.g., maintained, level of translation of an mRNA encoding the therapeutic payload or prophylactic payload.
E109. The system, or LNP composition of embodiment 94 or 95, wherein the system results in increased expression and/or level of the therapeutic payload or prophylactic payload, e.g., increased protein level, translation, or half-life, e.g., as measured by an assay of Example 4.
E110. The system, or LNP composition of embodiment 109, wherein the increased expression and/or level of the therapeutic payload or prophylactic payload is compared to an otherwise similar cell that has been contacted with an mRNA encoding the therapeutic payload or prophylactic payload which mRNA lacks a binding element of the first polynucleotide.
E111. The system, or LNP composition of embodiment 109 or 110, wherein the increase in expression and/or level of the therapeutic payload or prophylactic payload is about 1.5, 2, 3, 4, 5, 6, 7, 8, 9, 10, 20, 25, 30, 35, 40 or 50 fold.
E112. The system, or LNP composition of embodiment 94 or 95, wherein the system results in sustained expression and/or level of the therapeutic payload or prophylactic payload.
E113. The system, or LNP composition of any one of the preceding embodiments, wherein:

(a) the first polynucleotide comprises:
(1) a sequence encoding a therapeutic payload or prophylactic payload, and
(2) a binding element comprising an MS2 sequence, e.g., 6 MS2 sequences of 19 nucleotides separated by spacers of 20 nucleotides in length;

(b) the second polynucleotide comprises a sequence encoding:
(1) an effector molecule comprising eIF4G, e.g., wildtype eIF4G, a variant or a fragment thereof; and
(2) a tether molecule comprising MBP, e.g., wildtype MBP, a variant or fragment thereof.
E114. The system, of LNP composition of embodiment 113, wherein the first polynucleotide and the second polynucleotide each comprises an mRNA.
E115. The system, of LNP composition of embodiment 113, wherein the tether molecule of (b)(2) binds to, e.g., recognizes, the binding element of (a)(2).
E116. The system of embodiment 113, wherein the first polynucleotide is formulated as an LNP or the second polynucleotide is formulated as an LNP.
E117. The system of embodiment 113, wherein the first polynucleotide is formulated as an LNP and the second polynucleotide is formulated as an LNP.
E118. The system, or LNP composition of embodiment 117, wherein the LNP comprising the first polynucleotide and the LNP comprising the second polynucleotide are the same.
E119. The system, or LNP composition of embodiment 117, wherein the first polynucleotide and the second polynucleotide are each formulated as separate LNPs, optionally wherein:

(i) the LNP comprising the first polynucleotide is in a first composition and the LNP comprising the second polynucleotide is in a separate composition; or

(ii) the LNP comprising the first polynucleotide and the LNP comprising the second polynucleotide are in the same composition.
E120. The system, or LNP composition of any one of embodiments 113-119, wherein the binding element comprises a sequence comprising 19 nucleotides, e.g., a binding element nucleotide sequence provided in Table 2 or a sequence with at least 80%, 85%, 90%, 95%, 96%, 97%, 98%, 99% or 100% identity thereof.
E121. The system, or LNP composition of any one of embodiments 113-120, wherein the spacer sequence comprises 20 nucleotides, e.g., a spacer sequence provided in Table 2 or a sequence with at least 80%, 85%, 90%, 95%, 96%, 97%, 98%, 99% or 100% identity thereof.
E122. The system, or LNP composition of any one of embodiments 113-121, wherein the effector molecule comprises an amino acid sequence provided in Table 2, or a sequence with at least 80%, 85%, 90%, 95%, 96%, 97%, 98%, 99% or 100% identity thereof.
E123. The system, or LNP composition of any one of embodiments 113-122, wherein the effector molecule is encoded by a nucleotide sequence provided in Table 2, or a sequence with at least 80%, 85%, 90%, 95%, 96%, 97%, 98%, 99% or 100% identity thereof.
E124. The system, or LNP composition of any one of embodiments 113-123, wherein the tether molecule comprises an amino acid sequence provided in Table 2 or a sequence with at least 80%, 85%, 90%, 95%, 96%, 97%, 98%, 99% or 100% identity thereof.
E125. The system, or LNP composition of any one of embodiments 113-124, wherein the tether molecule is encoded by a nucleotide sequence provided in Table 2 or a sequence with at least 80%, 85%, 90%, 95%, 96%, 97%, 98%, 99% or 100% identity thereof.
E126. The system, or LNP composition of any one of the preceding embodiments, wherein the first polynucleotide comprises an mRNA comprising at least one chemical modification.
E127. The system, or LNP composition of any one of the preceding embodiments, wherein the second polynucleotide comprises an mRNA comprising at least one chemical modification.
E128. The system, or LNP composition of embodiment 126 or 127 , wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl -pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.
E129. The system, or LNP composition of embodiment 128, wherein the chemical modification is selected from the group consisting of pseudouridine, N1-methylpseudouridine, 5-methylcytosine, 5-methoxyuridine, and a combination thereof.
E130. The system, or LNP composition of embodiment 128, wherein the chemical modification is N1-methylpseudouridine.
E131. The LNP composition of any one of the preceding embodiments, wherein the mRNA comprises fully modified N1-methylpseudouridine.
E132. The system of any one of embodiments 126-131, or the LNP composition of any one of the preceding embodiments, wherein the LNP composition comprises: (i) an ionizable lipid, e.g., an amino lipid; (ii) a sterol or other structural lipid; (iii) a non-cationic helper lipid or phospholipid; and (iv) a PEG-lipid.
E133. The system or LNP composition of embodiment 132, wherein the ionizable lipid comprises an amino lipid.
E134. The system or LNP composition of embodiment 132 or 133, wherein the ionizable lipid comprises a compound of any of Formulae (I), (IA), (IB), (II), (IIa), (IIb), (IIc), (IId), (IIe), (IIf), (IIg), (III), (IIIa1), (IIIa2), (IIIa3), (IIIa4), (IIIa5), (IIIa6), (IIIa7), or (IIIa8).
E135. The system or LNP composition of any one of embodiments 132-134, wherein the ionizable lipid comprises a compound of Formula (I).
E136. The system or LNP composition of any one of embodiments 132-135, wherein the ionizable lipid comprises a compound of Formula (IIa).
E137. The system or LNP composition of any one of embodiments 132-135, wherein the ionizable lipid comprises a compound of Formula (IIe).
E138. The system or LNP composition of any one of embodiments 132-137, wherein the non-cationic helper lipid or phospholipid comprises a compound selected from the group consisting of DSPC, DPPC, or DOPC.
E139. The system or LNP composition of 138, wherein the phospholipid is DSPC, e.g., a variant of DSPC, e.g., a compound of Formula (IV).
E140. The system or LNP composition of any one of embodiments 132-141, wherein the structural lipid is chosen from alpha-tocopherol, β-sitosterol or cholesterol.
E141. The system or LNP composition of embodiment 140, wherein the structural lipid is alpha-tocopherol.
E142. The system or LNP composition of embodiment 140, wherein the structural lipid is β-sitosterol.
E143. The system or LNP composition of embodiment 140, wherein the structural lipid is cholesterol.
E144. The system or LNP composition of any one of embodiments 132-143, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
E145. The system or LNP composition of embodiments 144, wherein the PEG lipid is selected from the group consisting of PEG-c-DOMG, PEG-DMG, PEG-DLPE, PEG-DMPE, PEG-DPPC and PEG-DSPE lipid.
E146. The system or LNP composition of embodiment 145, wherein the PEG-lipid is PEG-DMG.
E147. The system or LNP composition of embodiments 144, wherein the PEG lipid is chosen from a compound of: Formula (V), Formula (VI-A), Formula (VI-B), Formula (VI-C) or Formula (VI-D).
E148. The system or LNP composition of embodiment 147, wherein the PEG lipid is a compound of Formula (VI-A).
E149. The system or LNP composition of embodiment 147, is a compound of Formula (VI-B).
E150. The system or LNP composition of any one of embodiments 132-149, wherein the LNP comprises a molar ratio of about 20-60% ionizable lipid: 5-25% phospholipid: 25-55% cholesterol; and 0.5-15% PEG lipid.
E151. The system or LNP composition of embodiment 149, wherein the LNP comprises a molar ratio of about 50% ionizable lipid: about 10% phospholipid: about 38.5% cholesterol; and about 1.5% PEG lipid.
E152. The system or LNP composition of embodiment 149 or 150, wherein the LNP comprises a molar ratio of about 49.83% ionizable lipid: about 9.83% phospholipid: about 30.33% cholesterol; and about 2.0% PEG lipid.
E153. The system or LNP composition of any one of the preceding embodiments, which is formulated for intravenous, subcutaneous, intramuscular, intranasal, intraocular, rectal, pulmonary or oral delivery.
E154. The system or LNP composition of any one of the preceding embodiments, which is formulated for intravenous delivery.
E155.The system or LNP composition of any one of the preceding embodiments, further comprising a pharmaceutically acceptable carrier or excipient.
E156. The system, or LNP composition of any one of the preceding embodiments, wherein the polynucleotide, e.g., the first and/or second polynucleotide comprises a cap, a 3′ UTR, a 5′ UTR, a Poly A tail and/or a micro RNA (miRNA) binding site.
E157. The system, or LNP composition of embodiment 156, wherein the cap comprises a cap disclosed herein.
E158 The system, or LNP composition of embodiment 156, wherein the polynucleotide, e.g., the first and/or second polynucleotide does not comprise a cap.
E159. The system, or LNP composition of any one of embodiments 156-158, wherein the 3′ UTR comprises a 3′ UTR disclosed herein, e.g., a v1.1 3′ UTR or a sequence with at least 80%, 85%, 90%, 95%, 96%, 97%, 98%, 99%, or 100% identity thereto.
E160. The system, or LNP composition of any one of embodiments 156-159, wherein the 5′ UTR comprises a 5′ UTR disclosed herein.
E161. The system, or LNP composition of any one of embodiments 156-160, wherein the Poly A tail comprises a Poly A tail sequence disclosed herein or a fragment thereof.
E162. The system, or LNP composition of any one of embodiments 156-160, wherein the polynucleotide, e.g., the first and/or second polynucleotide does not comprise a Poly A tail.
E163. The system, or LNP composition of any one of embodiments 156-162, wherein the miRNA binding site comprises a miRNA binding site disclosed herein.
E164. The system, or LNP composition of any one of the preceding embodiments, wherein the polynucleotide, e.g., the first and/or second polynucleotide is a circular polynucleotide.
E165. The system, or LNP composition of embodiment E165, wherein the first polynucleotide is a circular polynucleotide.
E166. The system, or LNP composition of embodiment E165, wherein the second polynucleotide is a circular polynucleotide.
E167. A pharmaceutical composition comprising the system, or LNP composition of any one of the preceding embodiments.
E168. A cell comprising a system, or LNP composition of any one of the preceding embodiments.
E169. The cell of embodiment 168, which has been contacted with the system, or LNP composition.
E170. The cell of embodiment 168 or 169, which is maintained under conditions sufficient to allow for expression of one or both polynucleotides of the system, or LNP composition.
E170. A method of increasing expression of a therapeutic payload or prophylactic payload in a cell, comprising administering to the cell a system, or LNP composition of any one of embodiments 1-166.
E171. The LNP composition or system of any one of embodiments 1-166, for use in a method of increasing expression of a therapeutic payload or prophylactic payload in a cell.
E172. A method of increasing expression of a therapeutic payload or prophylactic payload, in a subject, comprising administering to the subject an effective amount of a system or LNP composition of any one of embodiments 1-166.
E173. The LNP composition or system of any one of embodiments 1-166, for use in a method of increasing expression of a therapeutic payload or prophylactic payload in a subject.
E174. A method of delivering a system, or LNP composition of any one of embodiments 1-166, to a cell.
E175. The LNP composition or system of any one of embodiments 1-166, for use in a method of delivering the system or LNP composition to a cell.
E176. The method of embodiment 174, or the LNP composition or system for use of embodiment 175, comprising contacting the cell in vitro, in vivo or ex vivo with the system or LNP composition.
E177. A method of delivering a system or LNP composition of any one of embodiments 1-166, to a subject having a disease or disorder, e.g., as described herein.
E178. The LNP composition or system of any one of embodiments 1-166, for use in a method of delivering the system or LNP composition to a subject having a disease or disorder, e.g., as described herein.
E179. A method of modulating an immune response in a subject, comprising administering to the subject in need thereof an effective amount of a system, or LNP composition of any one of embodiments 1-166.
E180. The LNP composition or system of any one of embodiments 1-166, for use in a method of modulating an immune response in a subject, comprising administering to the subject an effective amount of the system, or LNP composition.
E181. A method of treating, preventing, or preventing a symptom of, a disease or disorder comprising administering to a subject in need thereof an effective amount of a system, or LNP composition of any one of embodiments 1-166.
E182. The LNP composition or system of any one of embodiments 1-166, for use in a method of treating, preventing, or preventing a symptom of, a disease or disorder in a subject, comprising administering to the subject in need thereof an effective amount of the system, or LNP composition.
E184. The method, or system for use of any one of embodiments 170-183, wherein the first polynucleotide and/or the second polynucleotide of the system is formulated as an LNP.
E185. The method of embodiment 184, wherein both the first and the second polynucleotides of the system are each formulated as LNPs, e.g., the same or different LNPs.
E186. The method, or the LNP composition or system for use of any one of embodiments 170-185, wherein the LNP comprising (a) and the LNP comprising (b) are administered simultaneously, e.g., substantially simultaneously.
E187. The method, or the LNP composition or system for use of any one of embodiments 170-185, wherein the LNP comprising (a) and the LNP comprising (b) are administered sequentially.
E188. The method, or the LNP composition or system for use of embodiment 187, wherein the LNP comprising (a) is administered first.
E189. The method, or the LNP composition or system for use of embodiment 187 or 188, wherein the LNP comprising (a) is administered first followed by administration of the LNP comprising (b).
E190. The method, or the LNP composition or system for use of embodiment 187, wherein the LNP comprising (b) is administered first.
E191. The method, or the LNP composition or system for use of embodiment 187 or 190, wherein the LNP comprising (b) is administered first followed by administration of the LNP comprising (a).
E192. The method, or the LNP composition or system for use of any one of embodiments 170-191, wherein the LNP comprising the first polynucleotide is administered at a lower dose compared to a reference LNP.
E193. The method, or the LNP composition or system for use of embodiment 192, wherein the LNP comprising the first polynucleotide is administered at a dose that is at least 10%, 20%, 30%, 40%, 50%, 60%, 70%, 80%, or 90% lower compared to the dose of a reference LNP.
E194. The method, or the LNP composition or system for use of embodiment 192 or 193, wherein the reference LNP is chosen from: an otherwise similar LNP comprising a polynucleotide which does not have the binding element of the first polynucleotide; or an LNP that does not comprise the second polynucleotide.
E195. The method, or the LNP composition or system for use of any one of embodiments 170-191, wherein the LNP comprising the first polynucleotide is administered at a higher dose compared to the LNP comprising the second polynucleotide.
E196. The method or the LNP composition or system for use of embodiment 195, wherein the LNP comprising the first polynucleotide is administered at a dose that is at least 10%, 20%, 30%, 40%, 50%, 60%, 70%, 80%, or 90% higher compared to the dose of the LNP comprising the second polynucleotide.
E197. The method, or the LNP composition or system for use of any one of embodiments 170-191, wherein the LNP comprising the first polynucleotide is in molar excess compared to the LNP comprising the second polynucleotide.
E198. The method or the LNP composition or system for use of embodiment 197, wherein the LNP comprising the first polynucleotide is in about 1-800× molar excess, compared to the LNP comprising the second polynucleotide.
E199. The method or the LNP composition or system for use of embodiment 197 or 198, wherein the LNP comprising the first polynucleotide is in about 1-750×, about 2-700×, about 3-650×, about 4-600×, about 5-550×, about 6-500×, about 7-450×, about 8-400×, about 10-350×, about 15-300×, about 20-250×, about 25-200×, about 30-150×, about 35-100×, about 40-90×, about 45-80×, about 50-75×, about 60-70× molar excess compared to the LNP comprising the second polynucleotide.
E200. The method or the LNP composition or system for use of embodiment 197 or 198, wherein the LNP comprising the first polynucleotide is in about 2×, about 3×, about 4×, about 5×, about 6×, about 7×, about 8×, about 9×, about 10×, about 11×, about 12×, about 13×, about 14×, about 15×, about 20×, about 25×, about 30×, about 35×, about 40×, about 50×, about 60×, about 70×, about 80×, about 90×, about 100×, about 150×, about 200×, about 250×, about 300×, about 350×, about 400×, about 450×, about 500×, about 600×, about 650×, about 700×, about 750×, or about 800× molar excess compared to the LNP comprising the second polynucleotide.
E201. The method or the LNP composition or system for use of any one of embodiments 197-200, wherein the LNP comprising the first polynucleotide is in about 9× molar excess compared to the LNP comprising the second polynucleotide.
E202. The method or the LNP composition or system for use of any one of embodiments 197-200, wherein the LNP comprising the first polynucleotide is in about 10× molar excess compared to the LNP comprising the second polynucleotide.
E203. The method or the LNP composition or system for use of any one of embodiments 197-200, wherein the LNP comprising the first polynucleotide and the LNP comprising the second polynucleotide are at the same molar amounts.
E204. The method, or the LNP composition or system for use of any one of embodiments 171-203, which results in one, two, three, four, five, six or all, or any combination thereof, of the following in a cell (e.g., in a cell contacted with the system or LNP composition):
E205. The method, or the LNP composition or system for use of embodiment 204, wherein any one, or all of (i)-(vii) is compared to a cell which:

(a) has not been contacted with the system of embodiment 7;

(b) has not been contacted with the LNP composition of embodiment 1;

(c) has not been contacted with an LNP comprising the first polynucleotide; or

(d) has been contacted with an LNP comprising the first polynucleotide but has not been contacted with the second polynucleotide, e.g., an LNP comprising the second polynucleotide.
E206. The method, or the LNP composition or system for use of embodiment 204 or 205, wherein the system, or LNP composition results in increased expression and/or level of the mRNA encoding the therapeutic payload or prophylactic payload, e.g., as measured by an assay in Example 2 or 3.
E207. The method, or the LNP composition or system for use of embodiment 206, wherein the increased expression and/or level of the mRNA encoding the therapeutic payload or prophylactic payload is compared to an otherwise similar cell that has been contacted with an mRNA encoding the therapeutic payload or prophylactic payload which mRNA lacks a binding element of the first polynucleotide.
E208. The method, or the LNP composition or system for use of embodiment 206 or 207, wherein the increase in expression and/or level of the mRNA encoding the therapeutic payload or prophylactic payload is about 1.5, 2, 3, 4, 5, 6, 7, 8, 9, 10, 20, 25, 30, 35, 40 or 50 fold.
E209. The method, or the LNP composition or system for use embodiment 206 or 207, wherein the increase in expression and/or level of the mRNA comprises an increase in stability (e.g., half-life) of the mRNA encoding the therapeutic payload or prophylactic payload, e.g., about 1.5, 2, 3, 4, 5, 6, 7, 8, 9, 10, or 20 fold increase in stability of the mRNA encoding the therapeutic payload or prophylactic payload.
E210. The method, or the LNP composition or system for use of embodiment 209, wherein the mRNA encoding the therapeutic payload or prophylactic payload has a half-life of about 3-25 hours, about 4-20 hours, about 4-15 hours, about 5-10 hours, about 6-9 hours or about 7-8 hours.
E211. The method, or the LNP composition or system for use of embodiment 204 or 205, wherein the system or LNP composition results in sustained expression and/or level of the mRNA encoding the therapeutic payload or prophylactic payload, e.g., as measured by an assay in Example 4.
E212. The method, or the LNP composition or system for use of embodiment 211, wherein at least 15%, 20%, 30%, 40%, 50%, 60%, 70%, 80%, 90% or more of the expression and/or level of the mRNA encoding the therapeutic payload or prophylactic payload is sustained for a period of time, e.g., about 1, 2, 3, 4, 5, 6, 7, 8, 9, 10, 12, 24 or 36 hours.
E213. The method, or the LNP composition or system for use of embodiment 212, wherein the sustained expression and/or level of the mRNA encoding the therapeutic payload or prophylactic payload is compared to an otherwise similar cell that has been contacted with an mRNA encoding the therapeutic payload or prophylactic payload which mRNA lacks a binding element of the first polynucleotide.
E214. The method, or the LNP composition or system for use of embodiment 211, wherein the system, or LNP composition results in a decreased loss, e.g., about a 1.2-fold, 2-fold, 3-fold, 4-fold or 5-fold decrease in loss, of mRNA encoding the therapeutic payload or prophylactic payload.
E215. The method, or the LNP composition or system for use of embodiments 214, wherein the decrease in loss of mRNA encoding the therapeutic payload or prophylactic payload occurs over a period of time, e.g., about 1, 2, 3, 4, 5, 6, 7, 8, 9, 10, 12, or 24 hours.
E216. The method, or the LNP composition or system for use of embodiment 211, wherein the system, or LNP composition results in a decreased loss, e.g., about a 1.2-fold, 2-fold, 3-fold, 4-fold, 5-fold, 6-fold, 7-fold, 8-fold, 9-fold or 10-fold decrease in loss, of translating mRNA.
E217. The method, or the LNP composition or system for use of embodiment 216, wherein the decrease in loss of mRNA encoding the therapeutic payload or prophylactic payload occurs over a period of time, e.g., about 1, 2, 3, 4, 5, 6, 7, 8, 9, 10, 12, or 24 hours.
E218. The method, or the LNP composition or system for use of embodiment 211, wherein the system, or LNP composition results in a sustained, e.g., maintained, level of translation of an mRNA encoding the therapeutic payload or prophylactic payload.
E219. The method, or the LNP composition or system for use of embodiment 204 or 205, wherein the system results in increased expression and/or level of the therapeutic payload or prophylactic payload, e.g., increased protein level, translation, or half-life, e.g., as measured by an assay of Example 4.
E220. The method, or the LNP composition or system for use of embodiment 219, wherein the increased expression and/or level of the therapeutic payload or prophylactic payload is compared to an otherwise similar cell that has been contacted with an mRNA encoding the therapeutic payload or prophylactic payload which mRNA lacks a binding element of the first polynucleotide.
E221. The method, or the LNP composition or system for use of embodiment 219 or 220, wherein the increase in expression and/or level of the therapeutic payload or prophylactic payload is about 1.5, 2, 3, 4, 5, 6, 7, 8, 9, 10, 20, 25, 30, 35, 40 or 50 fold.
E222. The method, or the LNP composition or system for use of embodiment 204 or 205, wherein the system results in sustained expression and/or level of the therapeutic payload or prophylactic payload.
E223. The method, or the LNP composition or system for use of any one of embodiments 171-222, wherein the first polynucleotide comprises an mRNA comprising at least one chemical modification.
E224. The method, or the LNP composition or system for use of any one of embodiments 171-222, wherein the second polynucleotide comprises an mRNA comprising at least one chemical modification.
E225. The method, or the LNP composition or system for use of embodiment 223 or 224 , wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl -pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-0-methyl uridine.
E226. The method, or the LNP composition or system for use of embodiment 225, wherein the chemical modification is selected from the group consisting of pseudouridine, N1-methylpseudouridine, 5-methylcytosine, 5-methoxyuridine, and a combination thereof.
E227. The method, or the LNP composition or system for use of embodiment 225, wherein the chemical modification is N1-methylpseudouridine.
E228. The method, or the LNP composition or system for use of any one of embodiments 171-227, wherein the mRNA comprises fully modified N1-methylpseudouridine.
E229. The method, or the LNP composition or system for use of any one of embodiments 171-228, wherein the LNP composition comprises: (i) an ionizable lipid, e.g., an amino lipid; (ii) a sterol or other structural lipid; (iii) a non-cationic helper lipid or phospholipid; and (iv) a PEG-lipid.
E230. The method, or the LNP composition or system for use of embodiment 229, wherein the ionizable lipid comprises an amino lipid.
E231. The method, or the LNP composition or system for use of embodiment 229 or 230, wherein the ionizable lipid comprises a compound of any of Formulae (I), (IA), (IB), (II), (IIa), (IIb), (IIc), (IId), (IIe), (IIf), (IIg), (III), (IIIa1), (IIIa2), (IIIa3), (IIIa4), (IIIa5), (IIIa6), (IIIa7), or (IIIa8).
E232. The method, or the LNP composition or system for use of any one of embodiments 229-231, wherein the ionizable lipid comprises a compound of Formula
E233. The method, or the LNP composition or system for use of any one of embodiments 229-232, wherein the ionizable lipid comprises a compound of Formula (IIa).
E234. The method, or the LNP composition or system for use of any one of embodiments 229-232, wherein the ionizable lipid comprises a compound of Formula (IIe).
E235. The method, or the LNP composition or system for use of any one of embodiments 229-234, wherein the non-cationic helper lipid or phospholipid comprises a compound selected from the group consisting of DSPC, DPPC, or DOPC.
E236. The method, or the LNP composition or system for use of embodiment 235, wherein the phospholipid is DSPC, e.g., a variant of DSPC, e.g., a compound of Formula (IV).
E237. The method, or the LNP composition or system for use of any one of embodiments 229-238, wherein the structural lipid is chosen from alpha-tocopherol, β-sitosterol or cholesterol.
E238. The method, or the LNP composition or system for use of embodiment 237, wherein the structural lipid is alpha-tocopherol.
E239. The method, or the LNP composition or system for use of embodiment 237, wherein the structural lipid is β-sitosterol.
E240. The method, or the LNP composition or system for use of embodiment 237, wherein the structural lipid is cholesterol.
E241. The method, or the LNP composition or system for use of any one of embodiments 229-240, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.
E242. The method, or the LNP composition or system for use of embodiment 241, wherein the PEG lipid is selected from the group consisting of PEG-c-DOMG, PEG-DMG, PEG-DLPE, PEG-DMPE, PEG-DPPC and PEG-DSPE lipid.
E243. The method, or the LNP composition or system for use of embodiment 241 or 242, wherein the PEG-lipid is PEG-DMG.
E244. The method, or the LNP composition or system for use of any one of embodiments 229-240, wherein the PEG lipid is a compound chosen from: Formula (V), Formula (VI-A), Formula (VI-B), Formula (VI-C) or Formula (VI-D).
E245. The method, or the LNP composition or system for use of embodiment 244, wherein the PEG-lipid is a compound of Formula (VI-A).
E246. The method, or the LNP composition or system for use of embodiment 244, wherein the PEG-lipid is a compound of Formula (VI-B).
E247. The method, or the LNP composition or system for use of any one of embodiments 229-246, wherein the LNP comprises a molar ratio of about 20-60% ionizable lipid: 5-25% phospholipid: 25-55% cholesterol; and 0.5-15% PEG lipid.
E248. The method, or the LNP composition or system for use of embodiment 247, wherein the LNP comprises a molar ratio of about 50% ionizable lipid: about 10% phospholipid: about 38.5% cholesterol; and about 1.5% PEG lipid.
E249. The method, or the LNP composition or system for use of embodiment 247 or 248, wherein the LNP comprises a molar ratio of about 49.83% ionizable lipid: about 9.83% phospholipid: about 30.33% cholesterol; and about 2.0% PEG lipid.
E250. The method, or the LNP composition or system for use of any one of embodiments 171-249, wherein the LNP or system is formulated for intravenous, subcutaneous, intramuscular, intranasal, intraocular, rectal, pulmonary or oral delivery.
E251. The method, or the LNP composition or system for use of any one of embodiments 171-250, wherein the subject is a mammal, e.g., a human.
E252. The method, or the LNP composition or system for use of any one of embodiments 171-251, wherein the subject has a disease or disorder disclosed herein.
E253. The LNP composition of embodiment 2, wherein the effector molecule recognizes the binding element.
E254. The LNP composition of any one of embodiments 1-4, wherein the second polynucleotide is DNA.
E255. The LNP composition of embodiment 254, wherein the sequence encoding the effector molecule is under the control of a tissue-specific promoter.
E256. The LNP composition of any one of embodiments 1-4, wherein expression or recruitment of the effector molecule is under the control of a trigger in a specific microenvironment or specific cell-type.
E257. The LNP composition of embodiment 256, wherein the trigger is microRNA, receptor-mediated activation, and/or a change in pH and/or hypoxia.
E258. A system comprising:

(a) a first polynucleotide comprising: (1) a sequence encoding a therapeutic payload or a prophylactic payload, and (2) a binding element; and/or

(b) a second polynucleotide comprising a sequence encoding an effector molecule,

optionally wherein, (a) and (b) each comprise an mRNA.
E259. The system of embodiment E258, wherein the effector molecule further comprises a polypeptide that recognizes the binding element (a tether molecule).
E260. The system of embodiment E258, wherein the effector molecule recognizes the binding element.
E261. The system of any embodiment 7, wherein the second polynucleotide is DNA.
E262. The system of embodiment 7, wherein the effector molecule is under the control of a tissue-specific promoter.
E263. The system of embodiment 262, wherein expression of the effector molecule or recruitment of the effector molecule is under the control of a trigger in a specific microenvironment or specific cell-type.
E264. The system of embodiment 263, wherein the trigger is microRNA, receptor-mediated activation, and/or a change in pH and/or hypoxia.
E265. The system, or LNP composition of any one of embodiments 1-125 and 128-264, wherein the first polynucleotide comprises an mRNA which does not have any chemical modification.
E266. The system, or LNP composition of any one of embodiments 1-125 and 128-264, wherein the second polynucleotide comprises an mRNA which does not have any chemical modification.

### BRIEF DESCRIPTION OF THE DRAWINGS

FIG. 1 provides a schematic of the 2 RNA system. Target mRNA with MS2 loops in the 3′UTR and another mRNA encoding MS2-binding protein (MBP) fused to truncated eIF4G (eIF4GΔN) are co-delivered by transfection or electroporation. The MS2 loops either replace the 3′UTR completely or are added before or after v1.1_UTR sequence.

FIGS. 2A-2D depict increased protein output for target deg-GFP RNA in HeLa cells with tethered eIF4G. Total green intensity vs time is plotted. Target mRNA with MS2 loops in the 3′UTR was co-delivered with another mRNA encoding a control protein (EPO; SEQ ID NO: 81) or a tethered control (MBP-LacZ; SEQ ID NO: 39) or a tethered effector (MBP-eIF4GΔN; SEQ ID NO: 11). Experiments were done in 10× molar excess of target. FIG. 2A shows the results of a control target mRNA with no binding sites in the 3′UTR (the ‘3 UTR comprises a v1.1_sequence; SEQ ID NO: 4). FIG. 2B shows the results of a target mRNA with MS2 binding sites upstream of the v1.1_sequence in the 3’ UTR. FIG. 2C shows the results of a target mRNA with MS2 binding sites in the 3′ UTR. FIG. 2D shows the results of a target mRNA with MS2 binding sites downstream of the v1.1_sequence in the 3′ UTR. In this construct, the MS2 binding sites were adjacent to a PolyA sequence.

FIGS. 3A-3C depict increased protein output for target deg-GFP RNA in HeLa, HEK293 and Hep3b cells, with tethered eIF4G. Total AUC is plotted. 3′UTR status of the target mRNA is indicated on the x axis. Experiments were done in 3× molar excess of target. FIG. 3A shows deg-GFP RNA protein output in HeLa cells. FIG. 3B shows deg-GFP RNA protein output in HeLa cells. FIG. 3C shows deg-GFP RNA protein output in HeLa cells.

FIGS. 4A-4B shows that increasing the ratio of tethered effector increases the total protein output in HeLa cells, and half-life predicted by the model. 3′UTR status of the target deg-GFP mRNA is indicated in the schematic. FIG. 4A shows total green intensity over time. FIG. 4B shows predicted half-lives (hours) using the 4-parameter model. Values of half-life obtained under each condition are added as data labels. (Target mRNA was added at the same amount in all conditions, amounts of effector RNA are changing as shown).

FIGS. 5A-5B show Total green intensity vs time for target RNA co-delivered with different RNAs as depicted (nature of UTR is depicted on top of the panels, and FIGS. 5C and 5D show the % of the starting quantity of the target mRNA remaining in HEK293 cells with tethered eIF4G. HEK293 cells were electroporated with target reporter RNA and an mRNA encoding control protein (EPO; SEQ ID NO: 81); or an mRNA encoding tethered control protein (MBP-LacZ; SEQ ID NO: 39); or an mRNA encoding tethered effector protein (MBP-eIF4GΔN; SEQ ID NO: 11). The experiments were done in 3× molar excess of target RNA. FIGS. 5A-5B show that both the amount of protein and duration of expression was increased in the presence of the tethered effector for the target RNA with MS2 sites in the UTR. FIGS. 5C-5D show that the % of the starting quantity of the target mRNA with MS2 sites reduced much more slowly with time for tethered effector condition. This suggests an increase in half-life of the target RNA.

FIGS. 6A-6E depict maintenance of robust translation for target mRNAs at later time points in Hep3b with a tethered effector. Hep3b cells were imaged at the indicated time points after electroporation with target mRNA (MS2 loops containing UTR) with non-tethered control (EPO; SEQ ID NO: 81), tethered control (MBP-LacZ; SEQ ID NO: 39) or tethered effector (MBP-eIF4GΔN; SEQ ID NO: 11). All experiments were done with 1.5× molar target excess. Control cells were electroporated with Luciferase (FLuc) or no RNAs. Images were analyzed in the red and green channel for smFISH and V5 signal respectively. FIG. 6A shows tethered effector reduces the rate of target mRNA decay. FIG. 6B shows tethered effector reduces the loss of translating mRNAs with time. FIG. 6C shows a higher percentage of cells maintained robust translation at later time points with tethered effector. FIG. 6D shows tethered effector maintained translation per target mRNA over time. FIG. 6E shows similar data as FIG. 6D, average NPI intensity per cell is plotted. N in the figures represents the number of cells assessed. Colors represent target RNA transfection with RNA encoding non-tethered unrelated protein (black), tethered control protein (orange) and tethered effector protein (green).

FIGS. 7A-7D depict maintenance of robust translation for target mRNAs at later time points in HeLa with tethered effector. HeLa cells were imaged at the indicated time points after electroporation with target mRNA (MS2 loops containing UTR) with non-tethered control (EPO; SEQ ID NO: 81), tethered control (MBP-LacZ; SEQ ID NO: 39) or tethered effector (MBP-eIF4GΔN; SEQ ID NO: 11). All experiments were done with 1.5× molar target excess. Control cells were electroporated with Luciferase (Fluc) or no RNAs. Images were analyzed in the red and green channel for smFISH and V5 signal respectively. FIG. 7A shows no appreciable change in cytosolic mRNAs over time in any condition. FIG. 7B shows no appreciable change in translation per mRNA with time in any condition. FIG. 7C shows tethered effector maintained higher percentage of translating mRNAs over time. FIG. 7E shows a higher percentage of cells maintained robust translation at later time points with tethered eIF4G.

FIGS. 8A-8E depict the results of the experiments to identify the domain of eIF4G required for effector function. FIG. 8A provides a schematic of the constructs that were used. Target deg-GFP RNA was co-delivered with an mRNA encoding control protein, EPO (SEQ ID NO: 81); or an mRNA encoding tethered control protein, MBP-LacZ (SEQ ID NO: 39); or an mRNA encoding tethered effector protein, MBP-eIF4GΔN (SEQ ID NO: 11), or mRNAs encoding MBP-fused to eIF4G truncations and mutations as illustrated in FIG. 8A. The experiment was done in 10× molar excess of target RNA. FIGS. 8B and 8D show the results in HeLa cells and FIGS. 8C and 8E show the results in HEK293 cells.

FIG. 9 is a table depicting summary data of the binding site experiments of FIGS. 8A-8E.

FIGS. 10A-10C depict the tethering system and results of experiments done when the target RNA had 24MS2 sites. FIG. 10A is a schematic of the tethering system where RNA encoding target protein shows no detectable protein expression. This RNA can be activated upon interaction with a specific effector protein. The interaction is mediated by known RNA-binding protein-RNA interaction tether (MBP protein-MS2 stem loop structure in RNA). FIG. 10B depicts real time fluorescence curves for HeLa cells transfected with 2 RNAs. Each sample has target deg-GFP encoding RNA (3′ UTR_v1.1; SEQ ID NO: 4 or 3′UTR_24 MS2; SEQ ID NO: 154) co-transfected with a control (EPO; SEQ ID NO: 81) or MBP-effector protein (MBP-eIF4GdN; SEQ ID NO: 11) encoding RNA. FIG. 10C shows the AUC for the same data in FIG. 10A.

FIG. 11 provides a schematic of a system to recruit potential effectors to target RNA with an MS2 binding protein (MBP)-MS2 tether. The target mRNA has MS2 loops in the 3′UTR and lacks a polyA tail (A0). The second mRNA encodes MS2-binding protein (MBP) fused to an effector/eIF4G-mid (a truncated fragment of the eIF4G protein). The RNA-binding protein, MBP tethers the effector, eIF4G to target RNA via the recognition of the MS2 hairpins. This system can be coupled to a miRNA-dependent switch gate to permit tethering in specific cells, thereby turning ON expression of the target protein. nt: nucleotide; aa: amino acids.

FIG. 12A-12D depict that co-delivery with tethered effector rescues expression for tailless mRNAs in Hep3b or HeLa cells. Control (A100) target RNA (degGFP with v1.1_A100 tails) or test (A0) target RNA (degGFP with 6xMS2_A0 tails; SEQ ID NO: 3) were co-delivered with another mRNA encoding a control protein (t-ctrl; MBP-LacZ; SEQ ID NO: 39) or a tethered effector (t-eff; MBP-eIF4GMID2; SEQ ID NO: 23). Experiments were done in 10× molar excess of target. FIG. 12A shows the total green intensity over time in Hep3b cells co-transfected with the indicated RNA constructs. FIG. 12B is the AUC for the data in FIG. 12A, normalized to AUC obtained with standard A100. RNAs. FIG. 12C shows the total green intensity over time in HeLa cells co-transfected with the indicated RNA constructs. FIG. 12D is the AUC for the data in FIG. 12C, normalized to AUC obtained with standard A100 RNAs.

FIG. 13 shows that tailless target RNA shows detectable expression only when co-delivered with effector, and overall expression is further improved when tailless RNA is ligated to idT. The figure depicts the level of green fluorescent protein (GFP fluorescence, y-axis) measured at the indicated times (x-axis) after transfection of Hep3b cells with 3′v1.1_MS2_A0 or 3′v1.1_MS2-A0-idT deg-GFP constructs with Effector (t-eff; MBP-mid2; SEQ ID NO: 23) or Control (t-ctrl; MBP-LacZ; SEQ ID NO: 39) RNA.

FIG. 14 shows that capless target RNA shows detectable expression only when co-delivered with effector. The figure depicts the level of Relative Light Units (RLU, y-axis) measured at the indicated times (x-axis) after transfection of HeLa cells with 5′PPP-end NpiLUC constructs with Effector (t-eff; MBP-eIF4G-mid2; SEQ ID NO: 23) or Control (t-ctrl; MBP-eIF4G-mid2mut; SEQ ID NO: 69) RNA.

FIG. 15 shows that capless-tailless target RNA shows detectable expression only when co-delivered with effector. The figure depicts the level of Relative Light Units (RLU, y-axis) measured at the indicated times (x-axis) after transfection of HeLa cells with 5′PPP-A0 ends NpiLUC constructs with Effector (t-eff; MBP-eIF4G-mid2; SEQ ID NO: 23) or Control (t-ctrl; MBP-eIF4G-mid2mut; SEQ ID NO: 69) RNA.

FIG. 16 shows that tethered effector decreases loss of translating mRNAs over time. Hep3b cells were imaged at the indicated time points after electroporation with target mRNA (MS2 loops containing UTR) in combination with non-tethered control (nt-ctrl; EPO), tethered control (t-ctrl; MBP-LacZ; SEQ ID NO: 39) or tethered effector (t-eff; MBP-eIF4GΔN; SEQ ID NO: 11). The graph depicts the nascent peptide imaging (NPI)+smFISH +Spots count per cell. All experiments were done with 1.5× molar target excess. Control cells were electroporated with Luciferase (FLuc) or no RNAs. Images were analyzed in the red and green channel for smFISH and V5 signal respectively. The average number of spots positive for both Npi and smFISH per cell is plotted. Data represented as average +/−SEM.

FIGS. 17A-17C shows that the tethered effector maintains more translating mRNAs in more cells over time. Hep3b cells were electroporated with the target RNA (3′v1.1_MS2 A100 or 3′v1.1_A100) in combination with non-tethered control (nt-ctrl; LacZ; SEQ ID NO: 98), tethered control (t-ctrl; MBP-LacZ; SEQ ID NO: 39) or t-effector (MBP-eIF4GΔN; SEQ ID NO: 11). The graphs are cumulative frequency distribution plots showing the percentage of cells (y-axis) against the percentage of translating mRNAs (x-axis) at 4 hours (FIG. 17A), 8 hours (FIG. 17B) or 12 hours (FIG. 17C) post transfection. All experiments were done with 1.5× molar target excess. Control cells were electroporated with Luciferase (FLuc) or no RNAs. The average NPI intensity per cell is plotted at the various time points indicated (4 h, 8 h, 12 h).

FIG. 18 shows that tethering increases secreted protein expression and translation. v1.1_target RNA constructs (SEQ ID NO: 4) with optimized reading frames for the light chain and heavy chain pairs of two secreted antibodies, (Ab 1 and Ab2) were co-transfected into Hek293 cells with t-ctrl (MBP-LacZ; SEQ ID NO: 39) or t-eff (MBP-eIF4GΔN; SEQ ID NO: 11). Experiments were done with 5× molar target excess (Ab 1) or lx molar target excess (Ab2). In each of the antibody experiments, the tethered effector is tethered to two separate light and heavy chain encoding RNAs. The graphs show the concentration (μg/ml) of Ab 1 or Ab2 over time.

FIG. 19 provides a schematic of the single RNA tethering system. The mRNA molecule from 5′ to 3′ includes a CAP, a 5′UTR, target ORF, 3 protease cleavage sites in tandem: T2A-P2A-E2A/TPE (red), another ORF encoding for the RNA binding protein fused to an effector (orange-RBP, -green-Effector), and MS2 loops (orange stripes) in the 3′UTR. The MS2 loops after v1.1_UTR sequence. The TPE protease cleavage site leads to ribosome skipping during translation in a cell. The end product of this translation is that the target ORF encoded protein gets a C-terminal tag, and the RNA binding protein-effector fusion starts with a residual Proline (C-terminal amino acid) from TPE.

FIGS. 20A-20C show that the tethered single RNA system increases protein expression (Area under the curve) and overall duration of expression from target RNA. In this system, Balb/c mice are injected with an RNA construct containing a target (Luc) and effector/control separated by a TPE element (SEQ ID NOs: 94 and 96 respectively). FIG. 20A shows the level of luminescence over the various timepoints indicated for mice that were injected with a t-eff construct (MBP_eIF4G-mid2 (653-1130); Effector; SEQ ID NO: 23) or a t-ctrl construct (MBP-LacZ; Control; SEQ ID NO: 39). FIG. 20B shows the cumulative luminiscence plotted as total Area Under Curve; AUC) corresponding to FIG. 20A. FIG. 20C shows the luminescence over the time points indicated for mice that were injected with a t-eff construct (Effector) or a t-ctrl construct (Control). Data is shown as mean +/−SEM.

FIGS. 21A-21B depict increased protein output for target deg-GFP RNA in Hep3b cells with tethered effectors that are full length or truncations in eIF4G1 and eIF4G3. Total green intensity vs time is plotted. Target mRNA with v1.1_MS2 loops in the 3′UTR (SEQ ID NO: 1) was co-delivered with another mRNA encoding a control protein (LacZ) (SEQ ID NO: 98) or a tethered control (MBP-LacZ) (SEQ ID NO: 39) or different tethered effector proteins as depicted (SEQ ID NOs: 51, 55, 59, or 79). Experiments were done in 10× molar excess of target. FIG. 21A shows the results of a target mRNA with MS2 binding sites downstream of the v1.1sequence in the 3′ UTR. In this construct, the MS2 binding sites were adjacent to a PolyA sequence. FIG. 21B shows the total integrated area under the curve for the data in FIG. 22A.

FIGS. 22A-22B depict increased protein output for target deg-GFP RNA in Hep3b cells with tethered effectors that are different proteins that bind to (PABP) or can modulate the polyA tail (G1d2, TENT4A, TENT4B) of an RNA. Total green intensity vs time is plotted. Target mRNA with v1.1_MS2 loops in the 3′UTR (SEQ ID NO: 1) was co-delivered with another mRNA encoding a control protein (LacZ) or a tethered control (MBP-LacZ) (SEQ ID NO: 39) or different tethered effector proteins as depicted (SEQ ID NOs: 51, 55, 59, or 79). Experiments were done in 10× molar excess of target. FIG. 22A shows the results of a target mRNA with MS2 binding sites downstream of the v1.1sequence in the 3′ UTR. In this construct, the MS2 binding sites were adjacent to a PolyA sequence. FIG. 22B shows the total integrated area under the curve for the data in FIG. 22A.

### DETAILED DESCRIPTION

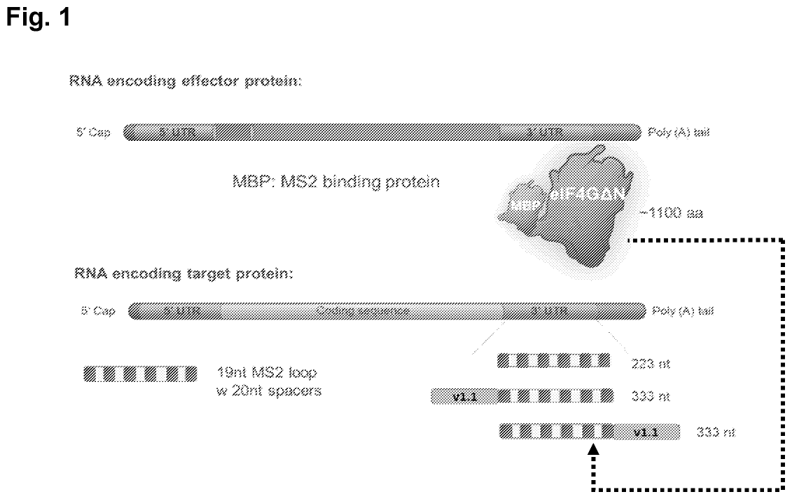

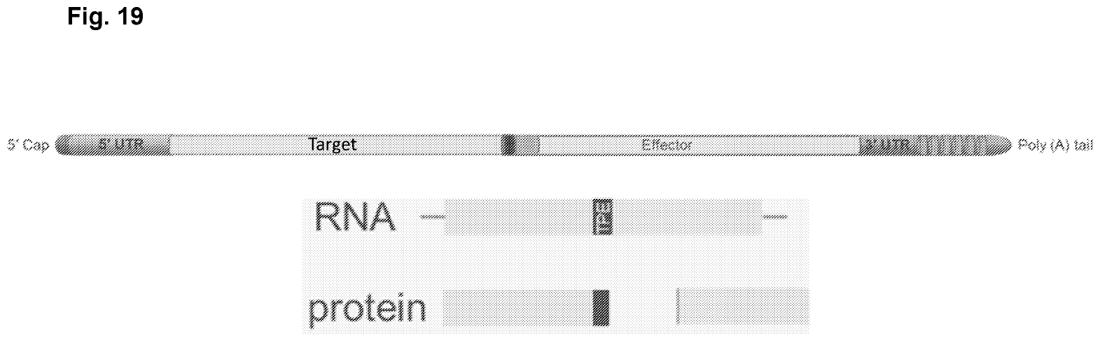

Efforts to increase mRNA potency have focused on generating canonical linear mRNAs with optimal sequence design for the untranslated regions (UTRs) and open reading frame (ORFs). Disclosed herein, inter alfa, is the discovery that RNA-binding-protein (RBP)-RNA interactions can modulate protein expression from exogenous mRNAs and/or mRNA sub-cellular localization. In some embodiments, the disclosure provides compositions and methods that can increase the efficacy, e.g., level and/or activity, of an mRNA, e.g., by making the mRNA independent of certain endogenous factors (e.g., by recruiting translation initiation factors). Additionally, disclosed herein is the discovery that effector molecules, e.g., effector proteins, can be recruited to an mRNA encoding a therapeutic payload or prophylactic payload to, e.g., promote desirable interactions between the mRNA and the effector molecule. In some embodiments, the effector molecule is recruited to the mRNA encoding the therapeutic payload or prophylactic payload by an RNA-binding protein, e.g., a tether molecule. Schematics of exemplary systems comprising these components are provided in FIG. 1 and FIG. 19.

Without wishing to be bound by theory it is believed that, in some embodiments, a system or LNP composition comprising: (1) an mRNA encoding a therapeutic payload or prophylactic payload; and (2) an mRNA encoding: an RBP, e.g., a tether molecule, and/or an effector molecule, utilizes the RBP-RNA interaction and allows for increased protein expression from the mRNA encoding the therapeutic payload or prophylactic payload.

Exemplary effects on mRNA expression with systems disclosed herein are provided in Examples 2-15. Example 2 shows increased potency of a target mRNA (e.g., increased protein expression and/or duration of protein expression) when co-delivered with an RNA encoding a tethered effector protein, e.g., tethered eIF4G. Examples 3 and 4 describe the increased half-life of a target mRNA when co-delivered with a tethered effector and the effects of a tethered effector on the translation of target mRNA analysis of the domains of eIF4G which are required for effector function is provided in Example 5. Example 6 shows that a tethered effector can rescue protein expression and increase mRNA stability. Examples 7 and 8 shows that tethered effector increases expression (and inferred mRNA stability) of a tailless RNA. Tailless target RNA with minimal-no baseline expression can be rescued (translation is restored) by effectors recruited using RBP-RNA (MBP-MS2) tethers. Examples 9-11 show that modified tailless and/or capless RNAs can be rescued by tethered effectors. Example 12 shows that tethered effector maintains more translating mRNAs over time. Example 13 shows that tetherhing increases secreted protein expression and translation. Example 14 shows that a single RNA tethering system where the target (i.e., therapeutic payload or prophylactic payload), and the tethered effector within the same RNA molecule, enhances target expression in vivo. Example 14 identifies other useful effector proteins or domains thereof for use in the systems disclosed herein.

Accordingly, disclosed herein are lipid nanoparticle (LNP) compositions or systems comprising a therapeutic payload or prophylactic payload, a binding element, a tether molecule and/or an effector molecule and uses thereof. The LNP compositions or systems of the present disclosure comprise: (a) a first polynucleotide (e.g., mRNA) comprising: (1) a sequence encoding a therapeutic payload or prophylactic payload, and (2) a binding element; and (b) a second polynucleotide (e.g., mRNA) comprising a sequence encoding: (1) an effector molecule, and/or (2) a polypeptide that recognizes the binding element (a tether molecule). In an embodiment, the effector molecule further comprises a tether molecule. In an embodiment, the In an embodiment the effector molecule polypeptide comprising a tether molecule comprises a first domain which modulates a parameter of, e.g., level and/or activity of: an RNA (e.g., an mRNA); or a protein encoded by the RNA. In an embodiment, the parameter comprises one, two, three or all of: (1) mRNA level and/or activity and/or subcellular localization (e.g., half-life and/or expression); (2) protein level and/or activity (e.g., half-life and/or expression); (3) protein translation rate or (4) protein localization, e.g., location. In an embodiment the effector molecule polypeptide comprising a tether molecule comprises a second domain which binds to, e.g., recognizes, the binding element (a tether molecule). In an embodiment the effector molecule comprising the tether molecule comprises a polypeptide comprising the first domain and the second domain. In an embodiment, the first and second domains are operatively linked.

In an embodiment, the first polynucleotide and the second polynucleotide are disposed in the same or different polynucleotides. In an embodiment, a system disclosed herein is formulated as an LNP. In an embodiment, the LNP comprising the first polynucleotide is formulated as an LNP. In some embodiments, the LNP comprising the second polynucleotide is formulated as an LNP. In an embodiment, the LNP comprising the first polynucleotide and the LNP comprising the second polynucleotide are the same LNP. In an embodiment, the LNP comprising the first polynucleotide and the LNP comprising the second polynucleotide are different LNPs. In an aspect, the LNP compositions or systems of the present disclosure can: increase the level and/or activity of the therapeutic payload or prophylactic payload, e.g., increase the level and/or activity of the mRNA encoding the therapeutic payload or prophylactic payload, or increase the level and/or activity of the therapeutic payload or prophylactic payload protein. In an aspect, the LNP compositions or systems can be used in a method of treating a disease or disorder, or for modulating an immune response in a subject.

### Definitions

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. Preferred means of administration are intravenous or subcutaneous.

Antibody molecule: In one embodiment, antibody molecules can be used for targeting to desired cell types. As used herein, “antibody molecule” refers to a naturally occurring antibody, an engineered antibody, or a fragment thereof, e.g., an antigen binding portion of a naturally occurring antibody or an engineered antibody. An antibody molecule can include, e.g., an antibody or an antigen-binding fragment thereof (e.g., Fab, Fab', F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). Exemplary antibody molecules include, but are not limited to, humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies;, Adnectins®; Affilins®; Trans-bodies®; Affibodies®; TrimerX®; MicroProteins; Fynomers®, Centyrins®; and KALBITOR®s.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of an LNP, “about” may mean +/−5% of the recited value. For instance, an LNP including a lipid component having about 40% of a given compound may include 30-50% of the compound.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., a nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle) and a cell may be contacted, for example, by adding the composition to the culture medium of the cell and may involve or result in transfection. Moreover, more than one cell may be contacted by a nanoparticle composition.

Delivering: As used herein, the term “delivering” means providing an entity to a destination. For example, delivering a therapeutic and/or prophylactic to a subject may involve administering a LNP including the therapeutic and/or prophylactic to the subject (e.g., by an intravenous, intramuscular, intradermal, pulmonary or subcutaneous route). Administration of a LNP to a mammal or mammalian cell may involve contacting one or more cells with the lipid nanoparticle.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Encapsulation efficiency: As used herein, “encapsulation efficiency” refers to the amount of a therapeutic and/or prophylactic that becomes part of a LNP, relative to the initial total amount of therapeutic and/or prophylactic used in the preparation of a LNP. For example, if 97 mg of therapeutic and/or prophylactic are encapsulated in a LNP out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

Effective amount: As used herein, the term “effective amount” of an agent is that amount sufficient to effect beneficial or desired results, for example, clinical results, and, as such, an “effective amount” depends upon the context in which it is being applied. For example, in the context of the amount of a target cell delivery potentiating lipid in a lipid composition (e.g., LNP) of the disclosure, an effective amount of a target cell delivery potentiating lipid is an amount sufficient to effect a beneficial or desired result as compared to a lipid composition (e.g., LNP) lacking the target cell delivery potentiating lipid. Non-limiting examples of beneficial or desired results effected by the lipid composition (e.g., LNP) include increasing the percentage of cells transfected and/or increasing the level of expression of a protein encoded by a nucleic acid associated with/encapsulated by the lipid composition (e.g., LNP). In the context of administering a target cell delivery potentiating lipid-containing lipid nanoparticle such that an effective amount of lipid nanoparticles are taken up by target cells in a subject, an effective amount of target cell delivery potentiating lipid-containing LNP is an amount sufficient to effect a beneficial or desired result as compared to an LNP lacking the target cell delivery potentiating lipid. Non-limiting examples of beneficial or desired results in the subject include increasing the percentage of cells transfected, increasing the level of expression of a protein encoded by a nucleic acid associated with/encapsulated by the target cell delivery potentiating lipid-containing LNP and/or increasing a prophylactic or therapeutic effect in vivo of a nucleic acid, or its encoded protein, associated with/encapsulated by the target cell delivery potentiating lipid-containing LNP, as compared to an LNP lacking the target cell delivery potentiating lipid. In some embodiments, a therapeutically effective amount of target cell delivery potentiating lipid-containing LNP is sufficient, when administered to a subject suffering from or susceptible to an infection, disease, disorder, and/or condition, to treat, improve symptoms of, diagnose, prevent, and/or delay the onset of the infection, disease, disorder, and/or condition. In another embodiment, an effective amount of a lipid nanoparticle is sufficient to result in expression of a desired protein in at least about 5%, 10%, 15%, 20%, 25% or more of target cells. For example, an effective amount of target cell delivery potentiating lipid-containing LNP can be an amount that results in transfection of at least 5%, 10%, 15%, 20%, 25%, 30%, or 35% of target cells after a single intravenous injection.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Fragment: A “fragment,” as used herein, refers to a portion. For example, fragments of proteins may include polypeptides obtained by digesting full-length protein isolated from cultured cells or obtained through recombinant DNA techniques. A fragment of a protein can be, for example, a portion of a protein that includes one or more functional domains such that the fragment of the protein retains the functional activity of the protein.

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is greater than about 50%. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises about 50% GC-content. In some embodiments of the disclosure, GC-rich polynucleotides, or any portions thereof, are exclusively comprised of guanine (G) and/or cytosine (C) nucleobases.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

Heterologous: As used herein, “heterologous” indicates that a sequence (e.g., an amino acid sequence or the polynucleotide that encodes an amino acid sequence) is not normally present in a given polypeptide or polynucleotide. For example, an amino acid sequence that corresponds to a domain or motif of one protein may be heterologous to a second protein.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC, where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Leaky scanning: A phenomenon known as “leaky scanning” can occur whereby the PIC bypasses the initiation codon and instead continues scanning downstream until an alternate or alternative initiation codon is recognized. Depending on the frequency of occurrence, the bypass of the initiation codon by the PIC can result in a decrease in translation efficiency. Furthermore, translation from this downstream AUG codon can occur, which will result in the production of an undesired, aberrant translation product that may not be capable of eliciting the desired therapeutic response. In some cases, the aberrant translation product may in fact cause a deleterious response (Kracht et al., (2017) Nat Med 23(4):501-507).

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Modified: As used herein “modified” refers to a changed state or structure of a molecule of the disclosure, e.g., a change in a composition or structure of a polynucleotide (e.g., mRNA). Molecules, e.g., polynucleotides, may be modified in various ways including chemically, structurally, and/or functionally. For example, molecules, e.g., polynucleotides, may be structurally modified by the incorporation of one or more RNA elements, wherein the RNA element comprises a sequence and/or an RNA secondary structure(s) that provides one or more functions (e.g., translational regulatory activity). Accordingly, molecules, e.g., polynucleotides, of the disclosure may be comprised of one or more modifications (e.g., may include one or more chemical, structural, or functional modifications, including any combination thereof). In one embodiment, polynucleotides, e.g., mRNA molecules, of the present disclosure are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence. In an embodiment, the mRNA is a circular mRNA.

Nanoparticle: As used herein, “nanoparticle” refers to a particle having any one structural feature on a scale of less than about 1000tn that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 mn. In exemplary embodiments, a nanoparticle is a particle having one or more dimensions of the order of about 1-1000 nm. In other exemplary embodiments, a nanoparticle is a particle having one or more dimensions of the order of about 10-500 nm. in other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like Although the size of most molecules would fit into the above outline, individual molecules are usually not referred to as nanoparticles.

Nucleic acid. As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside/Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group).

As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from an autoimmune disease, e.g., as described herein.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

RNA: As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal.

An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-liming group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Specific delivery: As used herein, the term “specific delivery,” “specifically deliver,” or “specifically delivering” means delivery of more (e.g., at least 10% more, at least 20% more, at least 30% more, at least 40% more, at least 50% more, at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic and/or prophylactic by a nanoparticle to a target cell of interest (e.g., mammalian target cell) compared to an off-target cell (e.g., non-target cells). The level of delivery of a nanoparticle to a particular cell may be measured by comparing the amount of protein produced in target cells versus non-target cells (e.g., by mean fluorescence intensity using flow cytometry, comparing the % of target cells versus non-target cells expressing the protein (e.g., by quantitative flow cytometry), comparing the amount of protein produced in a target cell versus non-target cell to the amount of total protein in said target cells versus non-target cell, or comparing the amount of therapeutic and/or prophylactic in a target cell versus non-target cell to the amount of total therapeutic and/or prophylactic in said target cell versus non-target cell. It will be understood that the ability of a nanoparticle to specifically deliver to a target cell need not be determined in a subject being treated, it may be determined in a surrogate such as an animal model (e.g., a mouse or NHP model).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Effector Molecule: As used herein, the term “effector molecule” refers to a molecule that can modulate a parameter of, e.g., level and/or activity of: an RNA (e.g., an mRNA); or a protein encoded by the RNA. In an embodiment, the parameter comprises one, two or all of: (1) mRNA level and/or activity and/or subcellular localization (e.g., half-life and/or expression); (2) protein level and/or activity (e.g., half-life and/or expression); (3) protein translation rate or (4) protein localization, e.g., location. In an embodiment, an effector molecule comprises: a translation factor, a splicing factor, an RNA stabilizing factor, an RNA editing factor, an RNA-binding factor, an RNA localizing factor, or any combination thereof, e.g., as provided in Table 4. An effector molecule comprises wildtype (e.g., naturally occurring, e.g., human), full length, a fragment (e.g., biologically active or functional fragment), or a variant of any of the aforementioned classes of effector molecules. In an embodiment, the effector molecule further comprises a tether molecule. In an embodiment the effector molecule polypeptide comprising a tether molecule comprises a first domain which modulates a parameter of, e.g., level and/or activity of: an RNA (e.g., an mRNA); or a protein encoded by the RNA, e.g., as described herein. In an embodiment the effector molecule polypeptide comprising a tether molecule comprises a second domain which binds to, e.g., recognizes, the binding element (a tether molecule). In an embodiment, the effector molecule comprises an RNA-binding protein or a fragment thereof. In an embodiment, an effector molecule comprises a translation factor, e.g., eIF4G. In an embodiment, an effector molecule comprises wildtype (e.g., naturally occurring, e.g., human), full length, a fragment (e.g., biologically active or functional fragment), or a variant of eIF4G. Exemplary eIF4G constructs are provided herein, e.g., in Table 2.

Binding element: As used herein, the term “binding element” refers to a nucleic acid sequence, e.g., a DNA or RNA sequence, which is recognized by a tether molecule. In an embodiment, the binding element forms a structure, e.g., a three-dimensional structure, e.g., a kink-turn, a loop, a stem or other known structure. Exemplary binding elements include, but are not limited, to those provided in Table 1.

Tether Molecule: As used herein, the term “tether molecule” refers to a molecule which binds to, e.g., recognizes, a binding element or a fragment thereof. In an embodiment, the tether molecule binds to, e.g., recognizes, a sequence, e.g., a DNA or RNA sequence, comprising the binding element, or fragment thereof. In an embodiment, the tether molecule binds to, e.g., recognizes, a structure comprising a sequence, e.g., a DNA or RNA sequence, comprising the binding element, or fragment thereof. In an embodiment, the effector molecule comprises an RNA-binding protein or a fragment thereof. Exemplary tether molecules are provided in Table 1.

Therapeutic Agent: The term “therapeutic agent” refers to any agent that, when administered to a subject, has a therapeutic, diagnostic, and/or prophylactic effect and/or elicits a desired biological and/or pharmacological effect. In some embodiments, the therapeutic agent comprises or is a therapeutic payload. In some embodiments, the therapeutic agent comprises or is a small molecule or a biologic (e.g., an antibody molecule).

Therapeutic payload or prophylactic payload: As used herein, the term “therapeutic payload or prophylactic payload” refers to an agent which elicits a desired biological and/or pharmacological effect. In an embodiment, the therapeutic payload or prophylactic payload has a therapeutic and/or prophylactic effect. In an embodiment, the therapeutic payload or prophylactic payload comprises a protein, a polypeptide, a peptide or a fragment (e.g., a biologically active fragment) thereof. In an embodiment, the therapeutic payload or prophylactic payload includes a sequence encoding a protein, e.g., a therapeutic protein. Some examples of therapeutic payload or prophylactic payloads may include, but are not limited to a secreted protein, a membrane-bound protein, or an intracellular protein. In an embodiment, the therapeutic payload or prophylactic payload includes a cytokine, an antibody, a vaccine (e.g., an antigen, or an immunogenic epitope), a receptor, an enzyme, a hormone, a transcription factor, a ligand, a membrane transporter, a structural protein, a nuclease, or a component, a variant or a fragment (e.g., a biologically active fragment) thereof. The terms protein, polypeptide and peptide are used interchangeably herein.

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

A “high uridine codon” is defined as a codon comprising two or three uridines, a “low uridine codon” is defined as a codon comprising one uridine, and a “no uridine codon” is a codon without any uridines. In some embodiments, a uridine-modified sequence comprises substitutions of high uridine codons with low uridine codons, substitutions of high uridine codons with no uridine codons, substitutions of low uridine codons with high uridine codons, substitutions of low uridine codons with no uridine codons, substitution of no uridine codons with low uridine codons, substitutions of no uridine codons with high uridine codons, and combinations thereof. In some embodiments, a high uridine codon can be replaced with another high uridine codon. In some embodiments, a low uridine codon can be replaced with another low uridine codon. In some embodiments, a no uridine codon can be replaced with another no uridine codon. A uridine-modified sequence can be uridine enriched or uridine rarefied.

Uridine Enriched: As used herein, the terms “uridine enriched” and grammatical variants refer to the increase in uridine content (expressed in absolute value or as a percentage value) in a sequence optimized nucleic acid (e.g., a synthetic mRNA sequence) with respect to the uridine content of the corresponding candidate nucleic acid sequence. Uridine enrichment can be implemented by substituting codons in the candidate nucleic acid sequence with synonymous codons containing less uridine nucleobases. Uridine enrichment can be global (i.e., relative to the entire length of a candidate nucleic acid sequence) or local (i.e., relative to a subsequence or region of a candidate nucleic acid sequence).

Uridine Rarefied: As used herein, the terms “uridine rarefied” and grammatical variants refer to a decrease in uridine content (expressed in absolute value or as a percentage value) in an sequence optimized nucleic acid (e.g., a synthetic mRNA sequence) with respect to the uridine content of the corresponding candidate nucleic acid sequence. Uridine rarefication can be implemented by substituting codons in the candidate nucleic acid sequence with synonymous codons containing less uridine nucleobases. Uridine rarefication can be global (i.e., relative to the entire length of a candidate nucleic acid sequence) or local (i.e., relative to a subsequence or region of a candidate nucleic acid sequence).

Variant: As used herein, the term “variant” refers to a molecule having at least 50%, 60%, 70%, 80%, 85%, 90%, 95%, 96%, 97%, 98%, 99% or 100% activity of the wild type molecule, e.g., as measured by an art-recognized assay.

Delivery of Therapeutic Payload or Prophylactic Payload with a Tethered Effector

Disclosed herein are, inter alia, LNP compositions or systems comprising a therapeutic payload or prophylactic payload, a binding element, a tether molecule and/or an effector molecule and uses thereof. In an embodiment, the LNP compositions of the present disclosure comprise: (a) a first polynucleotide (e.g., mRNA) comprising: (1) a sequence encoding a therapeutic payload or prophylactic payload, and (2) a binding element; and (b) a second polynucleotide (e.g., mRNA) comprising a sequence encoding: (1) an effector molecule, and/or (2) a polypeptide that recognizes the binding element (a tether molecule). In an embodiment, the effector molecule further comprises a tether molecule.

In an aspect, the LNP compositions or systems of the present disclosure can: increase the level and/or activity of the therapeutic payload or prophylactic payload, e.g., increase the level and/or activity of the mRNA encoding the therapeutic payload or prophylactic payload, increase the stability of the mRNA encoding the therapeutic payload or prophylactic payload, or increase the level and/or activity of the therapeutic payload or prophylactic payload protein. In an aspect, the LNP compositions of the present disclosure are contacted with cells, e.g., ex vivo or in vivo and can be used for treating a disease or disorder, for modulating an immune response in a subject, or to deliver a secreted polypeptide, an intracellular polypeptide or a transmembrane polypeptide to a subject.

In an embodiment, the first polynucleotide and the second polynucleotide are disposed in the same polynucleotide. In an embodiment, the first polynucleotide and the second polynucleotide are disposed in different polynucleotides.

In an aspect, the system disclosed herein is formulated as an LNP. In an embodiment, a system disclosed herein comprises (1) a polynucleotide, e.g., a first polynucleotide, encoding a therapeutic payload or prophylactic payload, e.g., as described herein; and/or (2) a polynucleotide, e.g., a second polynucleotide, encoding a tether molecule and an effector molecule. In an embodiment, the system comprising the first and/or second polynucleotides is formulated as an LNP.

In an aspect, the system disclosed herein is formulated as an LNP. In an embodiment, a system disclosed herein comprises (1) a polynucleotide, e.g., a first polynucleotide, encoding a therapeutic payload or prophylactic payload, e.g., as described herein; and/or (2) a polynucleotide, e.g., a second polynucleotide, encoding an effector molecule. In an embodiment, the effector molecule further comprises a tether molecule.

In an embodiment, the LNP comprising the first polynucleotide and the LNP comprising the second polynucleotide are the same, e.g., the same LNP comprises the first polynucleotide and the second polynucleotide.

In an embodiment, the LNP comprising the first polynucleotide and the LNP comprising the second polynucleotide are different, e.g., a first LNP comprises the first polynucleotide and a second LNP comprises the second polynucleotide.

In an embodiment, the LNP comprising the first polynucleotide is in a composition. In an embodiment, the LNP comprising the second polynucleotide is in a separate composition.

In an embodiment, the LNP comprising the first polynucleotide and the LNP comprising the second polynucleotide are in the same composition.

In aspects where a single RNA system is used (e.g., the system of FIG. 19), the first and second polynucleotides are in the same RNA molecule and can be separated by a protease cleavage site (e.g., a P2A, or T2A, E2A, or TPE (P2A-T2A-E2A) site) or an internal ribosomal entry site. The TPE region encodes for self cleaving peptides: The cleavage of the single RNA is triggered by ribosomal skipping of the peptide bond between the Proline (P) and Glycine (G) in C-terminal of 2A peptide, resulting in the peptide located upstream of the 2A peptide (i.e., the target peptide) to have extra amino acids on its C-terminal end while the peptide located downstream the 2A peptide (i.e., the tethered effector) will have an extra Proline on its N-terminal end. The molecular mechanism of 2A-peptide-mediated cleavage is believed to involve ribosomal “skipping” of glycyl-prolyl peptide bond formation rather than true proteolytic cleavage. Liu et al, 2017 Scientific Reports 7: 2193.

The 2A self-cleaving peptides that can be used in the present disclosure include, but are not limited to the following:
T2A (GSG)EGRGSLLTCGDVEENPGP (SEQ ID NO: 89);
P2A (GSG)ATNFSLLKQAGDVEENPGP (SEQ ID NO: 90);
E2A (GSG)QCTNYALLKLAGDVESNPGP (SEQ ID NO: 91);and
F2A (GSG)VKQTLNFDLLKLAGDVESNPGP (SEQ ID NO: 92); or a combination of the above, such as TPE.

In an aspect, an LNP composition comprising a polynucleotide, e.g., a first polynucleotide encoding a therapeutic payload or prophylactic payload, and/or a polynucleotide, e.g., a second polynucleotide encoding a tether molecule and/or an effector molecule, comprises: (i) an ionizable lipid, e.g., an amino lipid; (ii) a sterol or other structural lipid; (iii) a non-cationic helper lipid or phospholipid; and (iv) a PEG-lipid.

In another aspect, the LNP compositions of the disclosure are used in a method of treating a disease or disorder, or in a method of inhibiting an immune response in a subject.

In an aspect, an LNP composition or system comprising (1) a polynucleotide, e.g., a first polynucleotide, encoding a therapeutic payload or prophylactic payload, e.g., as described herein; and/or (2) a polynucleotide, e.g., a second polynucleotide, encoding a tether molecule and an effector molecule, can be administered with an additional agent, e.g., as described herein.

### Therapeutic Payload or Prophylactic Payload

Disclosed herein, inter cilia, is a system or LNP comprising a polynucleotide encoding a therapeutic payload or a prophylactic payload. In some embodiments, the therapeutic payload or prophylactic payload comprises an mRNA encoding: a secreted protein; a membrane-bound protein; or an intercellular protein, or peptides, polypeptides or biologically active fragments thereof.

In some embodiments, the therapeutic payload or prophylactic payload comprises an mRNA encoding a secreted protein, or a peptide, a polypeptide or a biologically active fragment thereof. In some embodiments, the secreted protein comprises a cytokine, or a variant or fragment (e.g., a biologically active fragment) thereof. In some embodiments, the secreted protein comprises an antibody or a variant or fragment (e.g., a biologically active fragment) thereof. In some embodiments, the secreted protein comprises an enzyme or a variant or fragment (e.g., a biologically active fragment) thereof. In some embodiments, the secreted protein comprises a hormone or a variant or fragment (e.g., a biologically active fragment) thereof. In some embodiments, the secreted protein comprises a ligand, or a variant or fragment (e.g., a biologically active fragment) thereof. In some embodiments, the secreted protein comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the secreted protein comprises a growth factor or a component, variant or fragment (e.g., a biologically active fragment) thereof. In some embodiments, the secreted protein comprises an immune modulator, e.g., an immune checkpoint agonist or antagonist.

In some embodiments, the therapeutic payload or prophylactic payload comprises an mRNA encoding a membrane-bound protein, or a peptide, a polypeptide or a biologically active fragment thereof. In some embodiments, the membrane-bound protein comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the membrane-bound protein comprises a ligand, a variant or fragment (e.g., a biologically active fragment) thereof. In some embodiments, the membrane-bound protein comprises a membrane transporter, a variant or fragment (e.g., a biologically active fragment) thereof. In some embodiments, the membrane-bound protein comprises a structural protein, a variant or fragment (e.g., a biologically active fragment) thereof. In some embodiments, the membrane-bound protein comprises an immune modulator, e.g., an immune checkpoint agonist or antagonist.

In some embodiments, the therapeutic payload or prophylactic payload comprises an mRNA encoding an intracellular protein, or a peptide, a polypeptide or a biologically active fragment thereof. In some embodiments, the intracellular protein comprises an enzyme, or a variant or fragment (e.g., a biologically active fragment) thereof. In some embodiments, the intracellular protein comprises a hormone, or a variant or fragment (e.g., a biologically active fragment) thereof. In some embodiments, the intracellular protein comprises a cytokine, or a variant or fragment (e.g., a biologically active fragment) thereof. In some embodiments, the intracellular protein comprises a transcription factor, or a variant or fragment (e.g., a biologically active fragment) thereof. In some embodiments, the intracellular protein comprises a nuclease, or a variant or fragment (e.g., a biologically active fragment) thereof. In some embodiments, the intracellular protein comprises comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine. In some embodiments, the intracellular protein comprises a structural protein, or a variant or fragment (e.g., a biologically active fragment) thereof.

In some embodiments, the therapeutic payload or prophylactic payload is chosen from a cytokine, an antibody, a vaccine (e.g., an antigen, an immunogenic epitope), a receptor, an enzyme, a hormone, a transcription factor, a ligand, a membrane transporter, a structural protein, a nuclease, a growth factor, an immune modulator, or a component, variant or fragment (e.g., a biologically active fragment) thereof.

In some embodiments, the therapeutic payload or prophylactic payload comprises a cytokine, or a variant or fragment (e.g., a biologically active fragment) thereof.

In some embodiments, the therapeutic payload or prophylactic payload comprises an antibody or a variant or fragment (e.g., a biologically active fragment) thereof.

In some embodiments, the therapeutic payload or prophylactic payload comprises a vaccine (e.g., an antigen, an immunogenic epitope), or a component, variant or fragment (e.g., a biologically active fragment) thereof. In some embodiments, the vaccine is a prophylactic vaccine. In some embodiments, the vaccine is a therapeutic vaccine, e.g., a cancer vaccine.

In some embodiments, the therapeutic payload or prophylactic payload comprises a receptor, or a variant or fragment (e.g., a biologically active fragment) thereof.

In some embodiments, the therapeutic payload or prophylactic payload comprises an enzyme, or a variant or fragment (e.g., a biologically active fragment) thereof.

In some embodiments, the therapeutic payload or prophylactic payload comprises a hormone, or a variant or fragment (e.g., a biologically active fragment) thereof.

In some embodiments, the therapeutic payload or prophylactic payload comprises a growth factor, or a variant or fragment (e.g., a biologically active fragment) thereof.

In some embodiments, the therapeutic payload or prophylactic payload comprises a nuclease, or a variant or fragment (e.g., a biologically active fragment)

In some embodiments, the therapeutic payload or prophylactic payload comprises a transcription factor, or a variant or fragment (e.g., a biologically active fragment) thereof.

In some embodiments, the therapeutic payload or prophylactic payload comprises a ligand, or a variant or fragment (e.g., a biologically active fragment) thereof.

In some embodiments, the therapeutic payload or prophylactic payload comprises a membrane transporter, or a variant or fragment (e.g., a biologically active fragment) thereof.

In some embodiments, the therapeutic payload or prophylactic payload comprises a structural protein, or a variant or fragment (e.g., a biologically active fragment) thereof.

In some embodiments, the therapeutic payload or prophylactic payload comprises an immune modulator, or a variant or fragment (e.g., a biologically active fragment) thereof. In some embodiments, the immune modulator comprises an immune checkpoint agonist or antagonist.

In some embodiments, the therapeutic payload or prophylactic payload comprises a protein or peptide.

In some embodiments, the first polynucleotide that comprises a sequence encoding a therapeutic payload or a prophylactic payload and a binding element is inherently unstable, self-degrading and/or dormant due to the presence of an inactivating/destabilizing sequence or a degradation tag in the first polynucleotide. The first polynucleotide is subject to stabilization and/or protein expression when co-delivered with a second polynucleotide encoding a tethered effector that binds to the binding element.

In some embodiments, the first polynucleotide that comprises a sequence encoding a therapeutic payload or a prophylactic payload and a binding element does not have a polyA tail and is therefore inherently unstable and/or unable to translate. The first polynucleotide is subject to stabilization when co-delivered with a second polynucleotide encoding a tethered effector that binds to the binding element.

### Binding Element

Disclosed herein, inter alfa, is a system or LNP comprising a polynucleotide, e.g., a first polynucleotide, comprising a binding element. A binding element comprises a sequence, e.g., a DNA or RNA sequence, which is bound, e.g., recognized by, an RNA binding protein or a fragment thereof, e.g., a tether molecule, e.g., as disclosed herein. In some embodiments, the tether molecule binds to a sequence comprising the binding element, or a fragment thereof. In some embodiments, the tether molecule binds to a structure comprising the binding element, or a fragment thereof.

In some embodiments, the system or LNP comprises a second polynucleotide encoding an RNA binding protein or a fragment thereof, e.g., a tether molecule, which binds to, e.g., recognizes, the binding element of the first polynucleotide.

In some embodiments, the binding element of the first polynucleotide is situated upstream (5′) or downstream (3′), or in the open reading frame of the sequence encoding the therapeutic payload or prophylactic payload.

In some embodiments, the binding element of the first polynucleotide is situated upstream (5′) or downstream (3′) of a 5′ UTR of the first polynucleotide. In some embodiments, the binding element of the first polynucleotide is situated upstream (5′) or downstream (3′) of a 3′ UTR of the first polynucleotide. In some embodiments, the binding element of the first polynucleotide is situated downstream of a 3′ UTR of the first polynucleotide. In some embodiments, the binding element of the first polynucleotide is situated adjacent, e.g., next to, a Poly A tail.

In some embodiments, the binding element of the first polynucleotide is bound by the tether molecule of the second polynucleotide, e.g., an effector molecule further comprising a tether molecule.

In some embodiments, a tether molecule is chosen from a tether molecule provided in Table 1, e.g., MBP, PCP, Lambda N, U1A or PUF, 15.5 kd, or LARP7 or a variant or fragment thereof. In some embodiments, the binding element comprises a sequence which is bound, e.g., recognized, by the tether molecule. In some embodiments, the binding element comprises a sequence comprising a structure that is bound, e.g., recognized, by the tether molecule.

In some embodiments, the binding element is chosen from a binding element provided in Table 1, e.g., MS2, PP7, BoxB, U1A hairpin, PRE, a kink-turn forming sequence, 7sk, or a variant or fragment thereof. In some embodiments, the binding element is MS2. In some embodiments, the binding element is PP7. In some embodiments, the binding element is BoxB. In some embodiments, the binding element is U1A hairpin. In some embodiments, the binding element is PRE. In some embodiments, the binding element is a kink-turn forming sequence. In some embodiments, the binding element is 7SK.

In some embodiments, when the binding element is MS2 (e.g., wildtype MS2, or a variant or fragment thereof) the tether molecule is MBP (e.g., wildtype MBP, a variant or fragment thereof).

In some embodiments, when the binding element is PP7 (e.g., wildtype PP7, or a variant or fragment thereof) the tether molecule is PCP (e.g., wildtype PCP, or a variant or fragment thereof).

In some embodiments, when the binding element is BoxB (e.g., wildtype BoxB, or a variant or fragment thereof) the tether molecule is Lambda N (e.g., wildtype Lambda N, or a variant or fragment thereof).

In some embodiments, when the binding element is U1A hairpin (e.g., wildtype U1A hairpin, or a variant or fragment thereof) the tether molecule is U1A (e.g., wildtype U1A, or a variant or fragment thereof).

In some embodiments, when the binding element is PRE (e.g., wildtype PRE, or a variant or fragment thereof) the tether molecule is PUF (e.g., wildtype PUF, or a variant or fragment thereof).

In some embodiments, when the binding element is a kink-turn forming sequence the tether molecule is 15.5 kd (e.g., wildtype 15.5 kd, or a variant or fragment thereof).

In some embodiments, when the binding element is a 7sk sequence the tether molecule is LARP7 (e.g., wildtype LARP7, or a variant or fragment thereof).

TABLE 1
Exemplary binding elements and tether molecules

| RNA binding protein (RBP) | RNA element | Recognition basis |
|---|---|---|
| PUF | PRE | Sequence |
| MBP | MS2 | Structure |
| PCP | PP7 | Structure |
| lambda N | boxB | Structure |
| U1A | U1A hairpin | Structure |
| 15.5kd | Kink-turn forming sequence | Structure |
| LARP7 | 7sk | Structure |

In some embodiments, the binding element comprises a sequence comprising 5, 6, 7, 8, 9, 10, 11, 12, 13, 14, 15, 16, 17, 18, 19, 20, 21, 22, 23, 24, 25, 30, 40, 50, 60, 70, 80, 90 or 100 nucleotides. In some embodiments, the binding element comprises a sequence comprising about 5-100, about 5-90, about 5-80, about 5-70, about 5-60, about 5-50, about 5-40, about 5-30, about 5-25, about 5-20, about 5-19, about 5-18, about 5-17, about 5-16, about 5-15, about 5-14, about 5-13, about 5-12, about 5-11, about 5-10, about 5-9, about 5-8, about 5-7 or about 5-6 nucleotides. In some embodiments, the binding element comprises a sequence comprising about 5-100, about 6-100, about 7-100, about 8-100, about 9-100, about 10-100, about 11-100, about 12-100, about 13-100, about 14-100, about 15-100, about 16-100, about 17-100, about 18-100, about 19-100, about 20-100, about 21-100, about 22-100, about 23-100, about 24-100, about 25-100, about 30-100, about 40-100, about 50-100, about 60-100, about 70-100, about 80-100, or about 90-100 nucleotides. In some embodiments, the binding element comprises a sequence comprising about 5-100, about 6-90, about 7-80, about 8-70, about 9-60, about 10-50, about 11-40, about 12-30, about 13-25, about 14-24, about 15-23, about 16-22, about 17-21, or about 18-20 nucleotides. In some embodiments, the binding element comprises a sequence comprising 19 nucleotides.

In some embodiments, the binding element comprises a binding element nucleotide sequence provided in Table 2 or a sequence with at least 80%, 85%, 90%, 95%, 96%, 97%, 98%, 99% or 100% identity thereof. In some embodiments, the binding element comprises a binding element sequence provided in SEQ ID NO: 1, SEQ ID NO: 2, SEQ ID NO: 3, SEQ ID NO: 4, SEQ ID NO: 154, or a sequence with at least 80%, 85%, 90%, 95%, 96%, 97%, 98%, 99% or 100% identity thereof.

In some embodiments of any of the systems, LNP compositions, methods or uses disclosed herein, the binding element comprises at least 1, 2, 3, 4, 5, 6, 7, 8, 9, 10, 11, 12, 13, 14, 15, 20 or 30 repeats of the sequence bound by the tether molecule of the second polynucleotide. In some embodiments, the binding element comprises no more than 80, 70, 60, 50, 40 or 30 repeats of the sequence bound by the tether molecule of the second polynucleotide. In some embodiments, the binding element comprises about 1-30, about 1-20, about 1-10, about 1-9, about 1-8, about 1-7, about 1-6, about 1-5, about 1-4, about 1-3, or about 1-2 repeats of the sequence bound by the tether molecule of the second polynucleotide. In some embodiments, the binding element comprises about 1-30, about 2-30, about 3-30, about 4-30 about, 5-30 about, 6-30, about 7-30, about 8-30, about 9-30, about 10-30, about 11-30, about 12-30, about 13-30, about 14-30, about 15-30, or about 20-30 repeats of the sequence bound by the tether molecule of the second polynucleotide. In some embodiments, the binding element comprises about 1-30, about 2-20, about 3-15, about 4-14, about 5-13, about 6-12, about 7-11, or about 8-10 repeats of the sequence bound by the tether molecule of the second polynucleotide. In some embodiments, the binding element comprises 6 repeats of the sequence bound by the tether molecule of the second polynucleotide.

In some embodiments of any of the systems, LNP compositions, methods or uses disclosed herein, each repeat is separated by a spacer sequence comprising 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90 or 100 nucleotides. In some embodiments, the spacer sequence comprises about 1-100, about 1-90, about 1-80, about 1-70, about 1-60, about 1-50, about 1-40, about 1-30, about 1-25, about 1-20, about 1-19, about 1-18, about 1-17, about 1-16, about 1-15, about 1-14, about 1-13, about 1-12, about 1-11, about 1-10, about 1-9, about 1-8, about 1-7, about 1-6, about 1-5, about 1-4, about 1-3, or about 1-2 nucleotides. In some embodiments, the spacer sequence comprises about 1-100, about 2-100, about 3-100, about 4-100, about 5-100, about 6-100, about 7-100, about 8-100, about 9-100, about 10-100, about 11-100, about 12-100, about 13-100, about 14-100, about 15-100, about 16-100, about 17-100, about 18-100, about 19-100, about 20-100, about 21-100, about 22-100, about 23-100, about 24-100, about 25-100, about 30-100, about 40-100, about 50-100, about 60-100, about 70-100, about 80-100, or about 90-100 nucleotides. In some embodiments, the spacer sequence comprises about 1-100, about 2-90, about 3-80, about 4-70, about 5-60, about 6-50, about 7-40, about 8-40, about 9-30, about 10-25, about 11-24, about 12-23, about 13-22, about 14-21, about 15-20, about 16-19, about 17-18 nucleotides. In some embodiment, the spacer sequence comprises 20 nucleotides.

In some embodiment, the spacer sequence comprises a spacer sequence provided in Table 2 or a sequence with at least 80%, 85%, 90%, 95%, 96%, 97%, 98%, 99% or 100% identity thereof.

TABLE 2
Exemplary sequences of a binding element,
a tether molecule, and/or an effector molecule.

| | | |
|---|---|---|
| SEQ | Sequence | |
| ID NO | information | Sequence |

3′UTR sequences

| | | |
|---|---|---|
| 1 | 3′v1.1_6xMS2 | TGATAATAGGCTGGAGCCTCGGTGGCCTAGCTTCTTGCCCCTTGG |
| | (MS2 sequences in | GCCTCCCCCCAGCCCCTCCTCCCCTTCCTGCACCCGTACCCCCGTGGT |
| | bold and underline) | CTTTGAATAAAGTCTGAGTGGGCGGCCAGGTATGATTACAACCTGT |
| | | GCTACTTTAGATGACTATTGAAAGACCATTAGGCTTTCTAACGGAC |
| | | GCTCACGAGAGAGGGGCACCAATAGGGCCCCACGGCACTTCAAAG |
| | | GTTTGGCTTGGAGTAGTAACCCAAGCAGCAACAGTTTTGACTTTCG |
| | | GACCACCATCAGGGGTCCCACGTTGGGAACACGTAACTCTCCTACT |
| | | AACAAGAGGAG |
| 2 | 3′6xMS2_3′v1.1 | TGATAATAGCAGGTATGATTACAACCTGTGCTACTTTAGATGA |
| | (MS2 sequences in | CTATTGAAAGACCATTAGGCTTTCTAACGGACGCTCACGAGAGAG |
| | bold and underline) | GGGCACCAATAGGGCCCCACGGCACTTCAAAGGTTTGGCTTGGAG |
| | | TAGTAACCCAAGCAGCAACAGTTTTGACTTTCGGACCACCATCAG |
| | | GGGTCCCACGTTGGGAACACGTAACTCTCCTACTAACAAGAGGAG |
| | | GCTGGAGCCTCGGTGGCCTAGCTTCTTGCCCCTTGGGCCTCCCCCCA |
| | | GCCCCTCCTCCCCTTCCTGCACCCGTACCCCCGTGGTCTTTGAATAAA |
| | | GTCTGAGTGGGCGGC |
| 3 | 3′_6xMS2 | TGATAATAGCAGGTATGATTACAACCTGTGCTACTTTAGATGA |
| | (MS2 sequences in | CTATTGAAAGACCATTAGGCTTTCTAACGGACGCTCACGAGAGAG |
| | bold and underline) | GGGCACCAATAGGGCCCCACGGCACTTCAAAGGTTTGGCTTGGAG |
| | | TAGTAACCCAAGCAGCAACAGTTTTGACTTTCGGACCACCATCAG |
| | | GGGTCCCACGTTGGGAACACGTAACTCTCCTACTAACAAGAGGAG |
| 4 | 3′v1.1 | TGATAATAGGCTGGAGCCTCGGTGGCCTAGCTTCTTGCCCCTTGG |
| | | GCCTCCCCCCAGCCCCTCCTCCCCTTCCTGCACCCGTACCCCCGTGGT |
| | | CTTTGAATAAAGTCTGAGTGGGCGGC |
| 154 | 3′UTR_24MS2 | TGATAATAGGCGCGATGATCACACGCGCACGCGAAGCTTGTGTT |
| | | CGCACGGCAAGCAGCAGCTGCCGTGGAGGCTTCCACGGCGAAATGT |
| | | CCACGAATACGGGACAAATAATTGATATAGGGCCGGCCCGTAAGAG |
| | | TACTACGGGATGATTCTCTTGACTTAAGTGCCGGACCACTAGGCCGG |
| | | CAACGGACAGACAAAGAGGCTCTGGGACTACCAAGCCCAGTCACAC |
| | | GGTACTTCAAGGCACCGGGAACAGTAGTCCCGGAGTGTTGACTTATA |
| | | GAAGTAGTCCCATTATTAAAGGGACTTCGGGACTTCCCTTCTCGCTC |
| | | CCGACCAGTAGGCGGGAAATGCGCCCTCCCGTACTGTCCTGCACCAT |
| | | TAGGGCAGGTTCTTTGAGGCTCTCTCGCTCGTGTATGAATACAACAC |
| | | GTGTGCGTCCTGTCCCCTCGTGACTCAGGACTACCGAGTCACAGCAA |
| | | ACTTAAAGGATCGCACGGACGAACACGCCGTGACATAGCTAATCAG |
| | | ACAATGGCACCAGTACCAACGGTGCACGTTGACACTCGAACGTGAT |
| | | TGACAGGATTACCGTCAACTCTTCCACTAGCCCGTTACTGGCAAGGA |
| | | TTACCTGCCACTGTTTTGTCTTAACCGGTTGACGTATCAACAGAACG |
| | | TCTGGTAGCAGCCACGCTTACTGGGCCATTAATAAAGGCCCAAGGA |
| | | ACTTAAAGCCAGTGCCCGGCACTATCAAGGCCGGGCATCATTGATTC |
| | | GACGCCGGACGGAGGAGCACCCCGTCGGGCATCGCGGCCCTGCGGG |
| | | TGCTAAACAGTAGTTAGCAGACATTTAGCGCCGGTTTGAAGCCCACG |
| | | AGTACGGGGCTTAGCAGCTCCAGATACACGGCGGGAACTACTAAGT |
| | | CCCGTAAACTCTCCACTATCGCCGGGCCAATCAATAGATGGCC |

5′ UTR sequence

| | | |
|---|---|---|
| 5 | 5′v1.1 | GGGAAATAAGAGAGAAAAGAAGAGTAAGAAGAAATATAAGAC |
| | | CCCGGCGCCGCCACC |

Effector molecule or tether molecule sequences

| | | |
|---|---|---|
| 6 | MBP (aa) | MASNFTQFVLVDNGGTGDVTVAPSNFANGIAEWISSNSRSQAYKV |
| | | TCSVRQSSAQNRKYTIKVEVPKGAWRSYLNMELTIPIFATNSDCELIVK |
| | | AMQGLLKDGNPIPSAIAANSGIY |
| 7 | MBP (nt) | ATGGCCAGCAACTTCACCCAGTTCGTGCTGGTGGACAACGGCGG |
| | | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTAC |
| 8 | eIF4GdN (aa) | IFASMQKPEGLPHISDVVLDKANKTPLRPLDPTRLQGINCGPDFTPSFAN |
| | | LGRTTLSTRGPPRGGPGGELPRGPAGLGPRRSQQGPRKEPRKIIATVLMT |
| | | EDIKLNKAEKAWKPSSKRTAADKDRGEEDADGSKTQDLFRRVRSILNK |
| | | LTPQMFQQLMKQVTQLAIDTEERLKGVIDLIFEKAISEPNFSVAYANMC |
| | | RCLMALKVPTTEKPTVTVNFRKLLLNRCQKEFEKDKDDDEVFEKKQKE |
| | | MDEAATAEERGRLKEELEEARDIARRRSLGNIKFIGELFKLKMLTEAIM |
| | | HDCVVKLLKNHDEESLECLCRLLTTIGKDLDFEKAKPRMDQYFNQMEK |
| | | IIKEKKTSSRIRFMLQDVLDLRGSNWVPRRGDQGPKTIDQIHKEAEMEE |
| | | HREHIKVQQLMAKGSDKRRGGPPGPPISRGLPLVDDGGWNTVPISKGSR |
| | | PIDTSRLTKITKPGSIDSNNQLFAPGGRLSWGKGSSGGSGAKPSDAASEA |
| | | ARPATSTLNRFSALQQAVPTESTDNRRVVQRSSLSRERGEKAGDRGDRL |
| | | ERSERGGDRGDRLDRARTPATKRSFSKEVEERSRERPSQPEGLRKAASL |
| | | TEDRDRGRDAVKREAALPPVSPLKAALSEEELEKKSKAIIEEYLHLNDM |
| | | KEAVQCVQELASPSLLFIFVRHGVESTLERSAIAREHMGQLLHQLLCAG |
| | | HLSTAQYYQGLYEILELAEDMEIDIPHVWLYLAELVTPILQEGGVPMGE |
| | | LFREITKPLRPLGKAASLLLEILGLLCKSMGPKKVGTLWREAGLSWKEF |
| | | LPEGQDIGAFVAEQKVEYTLGEESEAPGQRALPSEELNRQLEKLLKEGS |
| | | SNQRVFDWIEANLSEQQIVSNTLVRALMTAVCYSAIIFETPLRVDVAVL |
| | | KARAKLLQKYLCDEQKELQALYALQALVVTLEQPPNLLRMFFDALYD |
| | | EDVVKEDAFYSWESSKDPAEQQGKGVALKSVTAFFKWLREAEEESDH |
| | | N |
| 9 | eIF4GdN (nt) | ATATTCGCGAGCATGCAGAAGCCTGAAGGTCTGCCTCACATCAG |
| | | CGACGTGGTGCTGGACAAGGCCAACAAGACCCCTCTTAGACCTCTG |
| | | GACCCTACCAGACTGCAGGGCATCAACTGCGGCCCTGACTTCACTCC |
| | | TTCTTTCGCAAACCTGGGCAGAACCACCCTGAGCACCAGAGGCCCTC |
| | | CTAGAGGTGGTCCCGGCGGAGAACTCCCCAGGGGTCCTGCCGGCCT |
| | | GGGCCCTAGACGCTCCCAGCAAGGTCCTAGAAAGGAGCCAAGGAAG |
| | | ATCATCGCCACCGTGCTGATGACCGAGGACATCAAGCTGAACAAAG |
| | | CTGAGAAGGCCTGGAAGCCTAGCAGCAAGAGAACCGCCGCCGACAA |
| | | GGACAGAGGCGAGGAGGACGCCGACGGATCCAAGACCCAGGACCT |
| | | GTTCAGAAGAGTGAGAAGCATCCTCAACAAGCTGACCCCTCAGATG |
| | | TTCCAGCAGCTGATGAAGCAGGTGACGCAGCTCGCCATCGACACCG |
| | | AGGAGAGACTGAAGGGCGTGATCGACCTGATCTTTGAGAAGGCTAT |
| | | CTCAGAGCCTAACTTCAGCGTGGCCTACGCCAACATGTGCCGTTGCC |
| | | TGATGGCATTGAAGGTGCCAACCACCGAGAAGCCTACTGTGACCGT |
| | | CAATTTCCGTAAACTGCTGCTGAACCGGTGCCAGAAAGAGTTCGAG |
| | | AAGGATAAGGACGACGACGAGGTCTTCGAGAAGAAACAGAAAGAA |
| | | ATGGACGAGGCCGCCACCGCAGAGGAAAGGGGCCGATTAAAGGAG |
| | | GAGCTGGAGGAGGCCAGAGACATCGCCAGACGGCGTTCTCTGGGCA |
| | | ACATCAAGTTCATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTGCCGACCGAGAGCACCGACAACCGGCGAGTTGTGCAGAGAA |
| | | GCAGCCTGAGCAGAGAGAGGGGCGAGAAAGCCGGCGATAGAGGTG |
| | | ACCGACTGGAGAGAAGCGAGAGAGGAGGTGATAGAGGCGACCGGC |
| | | TTGACAGAGCCAGAACCCCTGCCACAAAGCGATCGTTCAGCAAGGA |
| | | GGTCGAAGAGAGGTCCAGGGAGCGCCCTAGCCAGCCGGAAGGACTC |
| | | AGAAAGGCAGCCAGTCTAACAGAGGACCGCGACAGGGGAAGAGAC |
| | | GCCGTGAAGAGGGAGGCCGCACTGCCTCCTGTGAGCCCTCTGAAGG |
| | | CGGCACTGAGCGAAGAAGAACTTGAGAAGAAGAGTAAGGCGATCAT |
| | | CGAGGAGTACCTGCACCTGAACGACATGAAGGAGGCCGTGCAGTGC |
| | | GTGCAAGAGCTCGCGAGCCCATCACTGCTGTTCATCTTCGTCCGGCA |
| | | CGGCGTGGAGTCCACACTGGAAAGATCTGCCATTGCTAGGGAGCAT |
| | | ATGGGCCAGTTGTTGCACCAATTGCTTTGCGCCGGCCACCTAAGTAC |
| | | TGCCCAGTACTATCAGGGTTTATACGAGATCCTCGAACTGGCCGAAG |
| | | ACATGGAGATCGACATCCCTCACGTGTGGCTGTACCTCGCAGAACTG |
| | | GTGACCCCTATCCTGCAGGAGGGCGGCGTTCCAATGGGTGAGTTGTT |
| | | CAGAGAAATCACAAAGCCACTGCGCCCACTGGGCAAGGCGGCTAGT |
| | | CTCCTTCTGGAGATTCTCGGCCTGCTCTGTAAGAGCATGGGTCCAAA |
| | | GAAGGTGGGCACCCTGTGGAGGGAAGCTGGACTCTCCTGGAAGGAA |
| | | TTCCTCCCTGAGGGTCAGGACATCGGCGCCTTCGTGGCCGAACAGAA |
| | | GGTTGAGTACACCCTGGGAGAGGAATCGGAAGCGCCGGGTCAGCGG |
| | | GCTCTGCCGAGTGAGGAGCTCAACAGACAACTCGAGAAGCTCCTCA |
| | | AGGAGGGATCCTCAAATCAGAGAGTGTTCGACTGGATCGAGGCCAA |
| | | TCTCAGCGAGCAGCAAATCGTGAGCAACACGTTGGTTCGCGCTCTCA |
| | | TGACAGCCGTGTGCTACTCAGCCATTATCTTCGAGACGCCTCTCCGC |
| | | GTGGACGTGGCAGTGCTCAAGGCCCGCGCTAAGCTGTTACAGAAGT |
| | | ACCTGTGCGACGAGCAGAAGGAACTGCAGGCCCTGTACGCCCTGCA |
| | | AGCTCTGGTCGTCACACTCGAGCAGCCTCCTAACCTGCTGAGAATGT |
| | | TCTTCGACGCATTGTACGACGAGGACGTTGTGAAGGAAGACGCTTTC |
| | | TACAGCTGGGAGTCTAGTAAGGATCCTGCGGAACAACAAGGCAAGG |
| | | GCGTTGCTCTAAAGAGCGTGACCGCCTTCTTCAAGTGGCTGCGAGAG |
| | | GCTGAGGAAGAGTCGGACCACAAC |
| 10 | MBP-eIF4GdN | MASNFTQFVLVDNGGTGDVTVAPSNFANGIAEWISSNSRSQAYKVTCS |
| | (aa) | VRQSSAQNRKYTIKVEVPKGAWRSYLNMELTIPIFATNSDCELIVKAMQ |
| | | GLLKDGNPIPSAIAANSGIYGGGGSIFASMQKPEGLPHISDVVLDKANKT |
| | | PLRPLDPTRLQGINCGPDFTPSFANLGRTTLSTRGPPRGGPGGELPRGPA |
| | | GLGPRRSQQGPRKEPRKIIATVLMTEDIKLNKAEKAWKPSSKRTAADKD |
| | | RGEEDADGSKTQDLFRRVRSILNKLTPQMFQQLMKQVTQLAIDTEERL |
| | | KGVIDLIFEKAISEPNFSVAYANMCRCLMALKVPTTEKPTVTVNFRKLL |
| | | LNRCQKEFEKDKDDDEVFEKKQKEMDEAATAEERGRLKEELEEARDIA |
| | | RRRSLGNIKFIGELFKLKMLTEAIMHDCVVKLLKNHDEESLECLCRLLT |
| | | TIGKDLDFEKAKPRMDQYFNQMEKIIKEKKTSSRIRFMLQDVLDLRGSN |
| | | WVPRRGDQGPKTIDQIHKEAEMEEHREHIKVQQLMAKGSDKRRGGPPG |
| | | PPISRGLPLVDDGGWNTVPISKGSRPIDTSRLTKITKPGSIDSNNQLFAPG |
| | | GRLSWGKGSSGGSGAKPSDAASEAARPATSTLNRFSALQQAVPTESTD |
| | | NRRVVQRSSLSRERGEKAGDRGDRLERSERGGDRGDRLDRARTPATKR |
| | | SFSKEVEERSRERPSQPEGLRKAASLTEDRDRGRDAVKREAALPPVSPL |
| | | KAALSEEELEKKSKAIIEEYLHLNDMKEAVQCVQELASPSLLFIFVRHGV |
| | | ESTLERSAIAREHMGQLLHQLLCAGHLSTAQYYQGLYEILELAEDMEID |
| | | IPHVWLYLAELVTPILQEGGVPMGELFREITKPLRPLGKAASLLLEILGLL |
| | | CKSMGPKKVGTLWREAGLSWKEFLPEGQDIGAFVAEQKVEYTLGEESE |
| | | APGQRALPSEELNRQLEKLLKEGSSNQRVFDWIEANLSEQQIVSNTLVR |
| | | ALMTAVCYSAIIFETPLRVDVAVLKARAKLLQKYLCDEQKELQALYAL |
| | | QALVVTLEQPPNLLRMFFDALYDEDVVKEDAFYSWESSKDPAEQQGK |
| | | GVALKSVTAFFKWLREAEEESDHN |
| 11 | MBP-eIF4GdN (nt) | ATGGCCAGCAACTTCACCCAGTTCGTGCTGGTGGACAACGGCGGTA |
| | | CCGGAGACGTGACCGTGGCCCCTTCTAACTTCGCCAACGGCATCGCC |
| | | GAGTGGATCAGCAGCAACAGCAGAAGCCAGGCCTACAAGGTGACCT |
| | | GCAGCGTGAGACAGAGCAGCGCCCAGAACAGAAAGTACACCATCAA |
| | | GGTGGAGGTGCCTAAGGGCGCCTGGAGAAGCTACCTGAACATGGAG |
| | | CTGACCATCCCTATCTTCGCCACCAACAGCGACTGCGAGCTGATCGT |
| | | GAAGGCCATGCAGGGCCTGCTGAAGGACGGCAACCCTATCCCTAGC |
| | | GCCATCGCCGCCAATTCAGGCATCTACGGAGGCGGTGGAAGCATAT |
| | | TCGCGAGCATGCAGAAGCCTGAAGGTCTGCCTCACATCAGCGACGT |
| | | GGTGCTGGACAAGGCCAACAAGACCCCTCTTAGACCTCTGGACCCTA |
| | | CCAGACTGCAGGGCATCAACTGCGGCCCTGACTTCACTCCTTCTTTC |
| | | GCAAACCTGGGCAGAACCACCCTGAGCACCAGAGGCCCTCCTAGAG |
| | | GTGGTCCCGGCGGAGAACTCCCCAGGGGTCCTGCCGGCCTGGGCCCT |
| | | AGACGCTCCCAGCAAGGTCCTAGAAAGGAGCCAAGGAAGATCATCG |
| | | CCACCGTGCTGATGACCGAGGACATCAAGCTGAACAAAGCTGAGAA |
| | | GGCCTGGAAGCCTAGCAGCAAGAGAACCGCCGCCGACAAGGACAG |
| | | AGGCGAGGAGGACGCCGACGGATCCAAGACCCAGGACCTGTTCAGA |
| | | AGAGTGAGAAGCATCCTCAACAAGCTGACCCCTCAGATGTTCCAGC |
| | | AGCTGATGAAGCAGGTGACGCAGCTCGCCATCGACACCGAGGAGAG |
| | | ACTGAAGGGCGTGATCGACCTGATCTTTGAGAAGGCTATCTCAGAGC |
| | | CTAACTTCAGCGTGGCCTACGCCAACATGTGCCGTTGCCTGATGGCA |
| | | TTGAAGGTGCCAACCACCGAGAAGCCTACTGTGACCGTCAATTTCCG |
| | | TAAACTGCTGCTGAACCGGTGCCAGAAAGAGTTCGAGAAGGATAAG |
| | | GACGACGACGAGGTCTTCGAGAAGAAACAGAAAGAAATGGACGAG |
| | | GCCGCCACCGCAGAGGAAAGGGGCCGATTAAAGGAGGAGCTGGAG |
| | | GAGGCCAGAGACATCGCCAGACGGCGTTCTCTGGGCAACATCAAGT |
| | | TCATAGGTGAGCTGTTCAAGCTAAAGATGCTCACCGAGGCCATAATG |
| | | CACGACTGCGTGGTGAAGCTACTGAAGAACCACGACGAAGAAAGCC |
| | | TGGAGTGCCTGTGCAGACTGCTGACCACCATCGGCAAGGACCTGGA |
| | | CTTCGAGAAGGCAAAGCCTCGAATGGACCAGTACTTCAACCAGATG |
| | | GAGAAGATTATCAAGGAGAAGAAGACCAGCAGCAGAATCAGATTCA |
| | | TGCTGCAGGACGTACTGGACCTGCGCGGAAGCAACTGGGTGCCAAG |
| | | GAGAGGGGACCAAGGACCAAAGACCATCGACCAGATCCACAAGGA |
| | | AGCGGAGATGGAGGAGCACAGAGAGCACATAAAGGTGCAGCAGCT |
| | | TATGGCCAAGGGCAGCGACAAGCGAAGAGGCGGCCCGCCCGGACCT |
| | | CCTATCAGCAGAGGCCTTCCTCTGGTAGACGACGGCGGCTGGAACA |
| | | CCGTGCCTATCTCTAAGGGCTCCAGACCTATCGACACTTCCCGTCTT |
| | | ACCAAGATCACCAAGCCAGGATCTATTGACAGCAACAACCAGCTGT |
| | | TCGCCCCAGGAGGAAGACTTAGCTGGGGCAAGGGAAGTTCCGGCGG |
| | | ATCCGGCGCCAAGCCTTCCGACGCCGCCAGCGAGGCTGCCAGACCT |
| | | GCCACCAGCACCTTGAACCGCTTTTCCGCTCTGCAGCAAGCTGTGCC |
| | | GACCGAGAGCACCGACAACCGGCGAGTTGTGCAGAGAAGCAGCCTG |
| | | AGCAGAGAGAGGGGCGAGAAAGCCGGCGATAGAGGTGACCGACTG |
| | | GAGAGAAGCGAGAGAGGAGGTGATAGAGGCGACCGGCTTGACAGA |
| | | GCCAGAACCCCTGCCACAAAGCGATCGTTCAGCAAGGAGGTCGAAG |
| | | AGAGGTCCAGGGAGCGCCCTAGCCAGCCGGAAGGACTCAGAAAGGC |
| | | AGCCAGTCTAACAGAGGACCGCGACAGGGGAAGAGACGCCGTGAA |
| | | GAGGGAGGCCGCACTGCCTCCTGTGAGCCCTCTGAAGGCGGCACTG |
| | | AGCGAAGAAGAACTTGAGAAGAAGAGTAAGGCGATCATCGAGGAG |
| | | TACCTGCACCTGAACGACATGAAGGAGGCCGTGCAGTGCGTGCAAG |
| | | AGCTCGCGAGCCCATCACTGCTGTTCATCTTCGTCCGGCACGGCGTG |
| | | GAGTCCACACTGGAAAGATCTGCCATTGCTAGGGAGCATATGGGCC |
| | | AGTTGTTGCACCAATTGCTTTGCGCCGGCCACCTAAGTACTGCCCAG |
| | | TACTATCAGGGTTTATACGAGATCCTCGAACTGGCCGAAGACATGGA |
| | | GATCGACATCCCTCACGTGTGGCTGTACCTCGCAGAACTGGTGACCC |
| | | CTATCCTGCAGGAGGGCGGCGTTCCAATGGGTGAGTTGTTCAGAGA |
| | | AATCACAAAGCCACTGCGCCCACTGGGCAAGGCGGCTAGTCTCCTTC |
| | | TGGAGATTCTCGGCCTGCTCTGTAAGAGCATGGGTCCAAAGAAGGT |
| | | GGGCACCCTGTGGAGGGAAGCTGGACTCTCCTGGAAGGAATTCCTC |
| | | CCTGAGGGTCAGGACATCGGCGCCTTCGTGGCCGAACAGAAGGTTG |
| | | AGTACACCCTGGGAGAGGAATCGGAAGCGCCGGGTCAGCGGGCTCT |
| | | GCCGAGTGAGGAGCTCAACAGACAACTCGAGAAGCTCCTCAAGGAG |
| | | GGATCCTCAAATCAGAGAGTGTTCGACTGGATCGAGGCCAATCTCA |
| | | GCGAGCAGCAAATCGTGAGCAACACGTTGGTTCGCGCTCTCATGAC |
| | | AGCCGTGTGCTACTCAGCCATTATCTTCGAGACGCCTCTCCGCGTGG |
| | | ACGTGGCAGTGCTCAAGGCCCGCGCTAAGCTGTTACAGAAGTACCT |
| | | GTGCGACGAGCAGAAGGAACTGCAGGCCCTGTACGCCCTGCAAGCT |
| | | CTGGTCGTCACACTCGAGCAGCCTCCTAACCTGCTGAGAATGTTCTT |
| | | CGACGCATTGTACGACGAGGACGTTGTGAAGGAAGACGCTTTCTAC |
| | | AGCTGGGAGTCTAGTAAGGATCCTGCGGAACAACAAGGCAAGGGCG |
| | | TTGCTCTAAAGAGCGTGACCGCCTTCTTCAAGTGGCTGCGAGAGGCT |
| | | GAGGAAGAGTCGGACCACAAC |
| 12 | MBP_eIF4G(fl) | MASNFTQFVLVDNGGTGDVTVAPSNFANGIAEWISSNSRSQAYKV |
| | (aa) | TCSVRQSSAQNRKYTIKVEVPKGAWRSYLNMELTIPIFATNSDCELIVK |
| | | AMQGLLKDGNPIPSAIAANSGIYGGGGSNKAPQSTGPPPAPSPGLPQPAF |
| | | PPGQTAPVVFSTPQATQMNTPSQPRQHFYPSRAQPPSSAASRVQSAAPA |
| | | RPGPAAHVYPAGSQVMMIPSQISYPASQGAYYIPGQGRSTYVVPTQQYP |
| | | VQPGAPGFYPGASPTEFGTYAGAYYPAQGVQQFPTGVAPTPVLMNQPP |
| | | QIAPKRERKTIRIRDPNQGGKDITEEIMSGARTASTPTPPQTGGGLEPQA |
| | | NGETPQVAVIVRPDDRSQGAIIADRPGLPGPEHSPSESQPSSPSPTPSPSPV |
| | | LEPGSEPNLAVLSIPGDTMTTIQMSVEESTPISRETGEPYRLSPEPTPLAEP |
| | | ILEVEVTLSKPVPESEFSSSPLQAPTPLASHTVEIHEPNGMVPSEDLEPEV |
| | | ESSPELAPPPACPSESPVPIAPTAQPEELLNGAPSPPAVDLSPVSEPEEQAK |
| | | EVTASMAPPTIPSATPATAPSATSPAQEEEMEEEEEEEEGEAGEAGEAES |
| | | EKGGEELLPPESTPIPANLSQNLEAAAATQVAVSVPKRRRKIKELNKKE |
| | | AVGDLLDAFKEANPAVPEVENQPPAGSNPGPESEGSGVPPRPEEADETW |
| | | DSKEDKIHNAENIQPGEQKYEYKSDQWKPLNLEEKKRYDREFLLGFQFI |
| | | FASMQKPEGLPHISDVVLDKANKTPLRPLDPTRLQGINCGPDFTPSFANL |
| | | GRTTLSTRGPPRGGPGGELPRGPAGLGPRRSQQGPRKEPRKIIATVLMTE |
| | | DIKLNKAEKAWKPSSKRTAADKDRGEEDADGSKTQDLFRRVRSILNKL |
| | | TPQMFQQLMKQVTQLAIDTEERLKGVIDLIFEKAISEPNFSVAYANMCR |
| | | CLMALKVPTTEKPTVTVNFRKLLLNRCQKEFEKDKDDDEVFEKKQKE |
| | | MDEAATAEERGRLKEELEEARDIARRRSLGNIKFIGELFKLKMLTEAIM |
| | | HDCVVKLLKNHDEESLECLCRLLTTIGKDLDFEKAKPRMDQYFNQMEK |
| | | IIKEKKTSSRIRFMLQDVLDLRGSNWVPRRGDQGPKTIDQIHKEAEMEE |
| | | HREHIKVQQLMAKGSDKRRGGPPGPPISRGLPLVDDGGWNTVPISKGSR |
| | | PIDTSRLTKITKPGSIDSNNQLFAPGGRLSWGKGSSGGSGAKPSDAASEA |
| | | ARPATSTLNRFSALQQAVPTESTDNRRVVQRSSLSRERGEKAGDRGDRL |
| | | ERSERGGDRGDRLDRARTPATKRSFSKEVEERSRERPSQPEGLRKAASL |
| | | TEDRDRGRDAVKREAALPPVSPLKAALSEEELEKKSKAIIEEYLHLNDM |
| | | KEAVQCVQELASPSLLFIFVRHGVESTLERSAIAREHMGQLLHQLLCAG |
| | | HLSTAQYYQGLYEILELAEDMEIDIPHVWLYLAELVTPILQEGGVPMGE |
| | | LFREITKPLRPLGKAASLLLEILGLLCKSMGPKKVGTLWREAGLSWKEF |
| | | LPEGQDIGAFVAEQKVEYTLGEESEAPGQRALPSEELNRQLEKLLKEGS |
| | | SNQRVFDWIEANLSEQQIVSNTLVRALMTAVCYSAIIFETPLRVDVAVL |
| | | KARAKLLQKYLCDEQKELQALYALQALVVTLEQPPNLLRMFFDALYD |
| | | EDVVKEDAFYSWESSKDPAEQQGKGVALKSVTAFFKWLREAEEESDH |
| | | N |
| 13 | MBP_eIF4G(fl) | ATGGCCAGCAACTTCACCCAGTTCGTGCTGGTGGACAACGGCGG |
| | (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCAA |
| | | CAAGGCCCCTCAGAGCACCGGCCCTCCTCCTGCCCCTAGCCCTGGCC |
| | | TGCCTCAGCCTGCCTTCCCTCCTGGCCAGACCGCCCCTGTGGTGTTCA |
| | | GCACCCCTCAGGCCACCCAGATGAACACACCTAGCCAGCCTAGACA |
| | | GCACTTCTACCCTAGCAGAGCCCAGCCTCCTAGCAGCGCCGCCAGCA |
| | | GAGTGCAGTCCGCTGCACCTGCCAGACCTGGCCCTGCCGCCCACGTG |
| | | TACCCTGCCGGCAGCCAGGTGATGATGATCCCGAGTCAAATCAGCTA |
| | | CCCTGCAAGCCAGGGCGCCTACTACATCCCGGGCCAGGGCAGAAGC |
| | | ACCTACGTGGTGCCTACCCAGCAGTACCCTGTGCAGCCTGGCGCCCC |
| | | TGGTTTCTATCCTGGCGCAAGCCCTACCGAGTTCGGAACTTACGCCG |
| | | GCGCTTACTATCCAGCTCAGGGCGTGCAGCAGTTCCCTACCGGCGTG |
| | | GCCCCTACCCCTGTGCTGATGAATCAGCCACCTCAGATCGCCCCTAA |
| | | GCGCGAGCGCAAGACCATCAGAATCCGCGATCCTAACCAGGGCGGC |
| | | AAGGACATCACCGAGGAGATCATGAGCGGCGCCAGAACAGCAAGTA |
| | | CTCCAACCCCGCCACAAACCGGCGGCGGCCTGGAGCCTCAAGCCAA |
| | | CGGCGAGACGCCACAAGTGGCCGTGATCGTACGCCCTGACGACCGG |
| | | AGCCAAGGTGCAATCATCGCCGATAGGCCTGGCCTCCCAGGTCCGG |
| | | AGCACAGCCCTAGCGAGTCCCAGCCGTCTTCACCATCACCAACCCCT |
| | | AGTCCATCCCCTGTTCTCGAACCAGGCAGCGAGCCTAACCTGGCCGT |
| | | GCTGAGCATACCAGGTGACACCATGACCACCATCCAGATGAGCGTG |
| | | GAGGAGAGCACCCCAATCAGCAGAGAAACTGGAGAGCCTTACAGAC |
| | | TGTCCCCAGAGCCGACCCCACTGGCCGAGCCAATACTGGAGGTGGA |
| | | GGTGACCCTGAGCAAGCCTGTGCCTGAGAGCGAGTTCAGCTCCTCTC |
| | | CACTGCAGGCCCCAACTCCTCTCGCAAGCCACACCGTGGAGATCCAC |
| | | GAACCTAACGGCATGGTACCAAGCGAAGATCTTGAGCCAGAGGTCG |
| | | AATCAAGCCCAGAACTGGCCCCTCCACCTGCCTGCCCGTCTGAATCT |
| | | CCGGTCCCTATCGCTCCTACGGCACAGCCTGAGGAGCTGCTGAACGG |
| | | TGCCCCGAGCCCTCCAGCAGTGGACTTATCCCCAGTATCAGAGCCTG |
| | | AAGAACAGGCCAAGGAGGTAACTGCCTCTATGGCGCCACCTACCAT |
| | | ACCTTCGGCAACACCGGCTACAGCACCATCTGCGACTAGTCCGGCTC |
| | | AGGAGGAGGAGATGGAGGAAGAAGAAGAGGAAGAGGAGGGCGAG |
| | | GCCGGAGAGGCCGGTGAAGCCGAGTCCGAGAAGGGCGGCGAAGAA |
| | | CTTCTCCCTCCAGAGTCAACTCCTATCCCTGCCAACCTTAGTCAGAAT |
| | | CTGGAGGCCGCCGCCGCTACTCAGGTTGCAGTGAGCGTGCCAAAGA |
| | | GACGTCGCAAGATCAAGGAGCTGAACAAGAAGGAGGCCGTGGGCG |
| | | ACCTGCTGGACGCCTTCAAGGAGGCAAACCCGGCGGTGCCTGAAGT |
| | | GGAGAATCAGCCTCCGGCCGGATCAAACCCTGGTCCTGAGAGTGAA |
| | | GGCAGCGGCGTCCCACCAAGACCTGAAGAGGCTGACGAAACTTGGG |
| | | ACAGCAAGGAGGACAAGATCCACAACGCCGAGAACATCCAGCCAG |
| | | GCGAGCAGAAGTACGAGTACAAGAGCGACCAGTGGAAGCCTCTTAA |
| | | CCTTGAAGAGAAGAAGAGATACGACAGAGAGTTCCTGCTGGGCTTC |
| | | CAGTTCATATTCGCGAGCATGCAGAAGCCTGAAGGTCTGCCTCACAT |
| | | CAGCGACGTGGTGCTGGACAAGGCCAACAAGACCCCTCTTAGACCT |
| | | CTGGACCCTACCAGACTGCAGGGCATCAACTGCGGCCCTGACTTCAC |
| | | TCCTTCTTTCGCAAACCTGGGCAGAACCACCCTGAGCACCAGAGGCC |
| | | CTCCTAGAGGTGGTCCCGGCGGAGAACTCCCCAGGGGTCCTGCCGG |
| | | CCTGGGCCCTAGACGCTCCCAGCAAGGTCCTAGAAAGGAGCCAAGG |
| | | AAGATCATCGCCACCGTGCTGATGACCGAGGACATCAAGCTGAACA |
| | | AAGCTGAGAAGGCCTGGAAGCCTAGCAGCAAGAGAACCGCCGCCGA |
| | | CAAGGACAGAGGCGAGGAGGACGCCGACGGATCCAAGACCCAGGA |
| | | CCTGTTCAGAAGAGTGAGAAGCATCCTCAACAAGCTGACCCCTCAG |
| | | ATGTTCCAGCAGCTGATGAAGCAGGTGACGCAGCTCGCCATCGACA |
| | | CCGAGGAGAGACTGAAGGGCGTGATCGACCTGATCTTTGAGAAGGC |
| | | TATCTCAGAGCCTAACTTCAGCGTGGCCTACGCCAACATGTGCCGTT |
| | | GCCTGATGGCATTGAAGGTGCCAACCACCGAGAAGCCTACTGTGAC |
| | | CGTCAATTTCCGTAAACTGCTGCTGAACCGGTGCCAGAAAGAGTTCG |
| | | AGAAGGATAAGGACGACGACGAGGTCTTCGAGAAGAAACAGAAAG |
| | | AAATGGACGAGGCCGCCACCGCAGAGGAAAGGGGCCGATTAAAGG |
| | | AGGAGCTGGAGGAGGCCAGAGACATCGCCAGACGGCGTTCTCTGGG |
| | | CAACATCAAGTTCATAGGTGAGCTGTTCAAGCTAAAGATGCTCACCG |
| | | AGGCCATAATGCACGACTGCGTGGTGAAGCTACTGAAGAACCACGA |
| | | CGAAGAAAGCCTGGAGTGCCTGTGCAGACTGCTGACCACCATCGGC |
| | | AAGGACCTGGACTTCGAGAAGGCAAAGCCTCGAATGGACCAGTACT |
| | | TCAACCAGATGGAGAAGATTATCAAGGAGAAGAAGACCAGCAGCA |
| | | GAATCAGATTCATGCTGCAGGACGTACTGGACCTGCGCGGAAGCAA |
| | | CTGGGTGCCAAGGAGAGGGGACCAAGGACCAAAGACCATCGACCA |
| | | GATCCACAAGGAAGCGGAGATGGAGGAGCACAGAGAGCACATAAA |
| | | GGTGCAGCAGCTTATGGCCAAGGGCAGCGACAAGCGAAGAGGCGGC |
| | | CCGCCCGGACCTCCTATCAGCAGAGGCCTTCCTCTGGTAGACGACGG |
| | | CGGCTGGAACACCGTGCCTATCTCTAAGGGCTCCAGACCTATCGACA |
| | | CTTCCCGTCTTACCAAGATCACCAAGCCAGGATCTATTGACAGCAAC |
| | | AACCAGCTGTTCGCCCCAGGAGGAAGACTTAGCTGGGGCAAGGGAA |
| | | GTTCCGGCGGATCCGGCGCCAAGCCTTCCGACGCCGCCAGCGAGGC |
| | | TGCCAGACCTGCCACCAGCACCTTGAACCGCTTTTCCGCTCTGCAGC |
| | | AAGCTGTGCCGACCGAGAGCACCGACAACCGGCGAGTTGTGCAGAG |
| | | AAGCAGCCTGAGCAGAGAGAGGGGCGAGAAAGCCGGCGATAGAGG |
| | | TGACCGACTGGAGAGAAGCGAGAGAGGAGGTGATAGAGGCGACCG |
| | | GCTTGACAGAGCCAGAACCCCTGCCACAAAGCGATCGTTCAGCAAG |
| | | GAGGTCGAAGAGAGGTCCAGGGAGCGCCCTAGCCAGCCGGAAGGA |
| | | CTCAGAAAGGCAGCCAGTCTAACAGAGGACCGCGACAGGGGAAGA |
| | | GACGCCGTGAAGAGGGAGGCCGCACTGCCTCCTGTGAGCCCTCTGA |
| | | AGGCGGCACTGAGCGAAGAAGAACTTGAGAAGAAGAGTAAGGCGA |
| | | TCATCGAGGAGTACCTGCACCTGAACGACATGAAGGAGGCCGTGCA |
| | | GTGCGTGCAAGAGCTCGCGAGCCCATCACTGCTGTTCATCTTCGTCC |
| | | GGCACGGCGTGGAGTCCACACTGGAAAGATCTGCCATTGCTAGGGA |
| | | GCATATGGGCCAGTTGTTGCACCAATTGCTTTGCGCCGGCCACCTAA |
| | | GTACTGCCCAGTACTATCAGGGTTTATACGAGATCCTCGAACTGGCC |
| | | GAAGACATGGAGATCGACATCCCTCACGTGTGGCTGTACCTCGCAG |
| | | AACTGGTGACCCCTATCCTGCAGGAGGGCGGCGTTCCAATGGGTGA |
| | | GTTGTTCAGAGAAATCACAAAGCCACTGCGCCCACTGGGCAAGGCG |
| | | GCTAGTCTCCTTCTGGAGATTCTCGGCCTGCTCTGTAAGAGCATGGG |
| | | TCCAAAGAAGGTGGGCACCCTGTGGAGGGAAGCTGGACTCTCCTGG |
| | | AAGGAATTCCTCCCTGAGGGTCAGGACATCGGCGCCTTCGTGGCCGA |
| | | ACAGAAGGTTGAGTACACCCTGGGAGAGGAATCGGAAGCGCCGGGT |
| | | CAGCGGGCTCTGCCGAGTGAGGAGCTCAACAGACAACTCGAGAAGC |
| | | TCCTCAAGGAGGGATCCTCAAATCAGAGAGTGTTCGACTGGATCGA |
| | | GGCCAATCTCAGCGAGCAGCAAATCGTGAGCAACACGTTGGTTCGC |
| | | GCTCTCATGACAGCCGTGTGCTACTCAGCCATTATCTTCGAGACGCC |
| | | TCTCCGCGTGGACGTGGCAGTGCTCAAGGCCCGCGCTAAGCTGTTAC |
| | | AGAAGTACCTGTGCGACGAGCAGAAGGAACTGCAGGCCCTGTACGC |
| | | CCTGCAAGCTCTGGTCGTCACACTCGAGCAGCCTCCTAACCTGCTGA |
| | | GAATGTTCTTCGACGCATTGTACGACGAGGACGTTGTGAAGGAAGA |
| | | CGCTTTCTACAGCTGGGAGTCTAGTAAGGATCCTGCGGAACAACAA |
| | | GGCAAGGGCGTTGCTCTAAAGAGCGTGACCGCCTTCTTCAAGTGGCT |
| | | GCGAGAGGCTGAGGAAGAGTCGGACCACAAC |
| 14 | MBP_eIF4G-dN | MASNFTQFVLVDNGGTGDVTVAPSNFANGIAEWISSNSRSQAYKV |
| | (623-1599, 3A) (aa) | TCSVRQSSAQNRKYTIKVEVPKGAWRSYLNMELTIPIFATNSDCELIVK |
| | | AMQGLLKDGNPIPSAIAANSGIYGGGGSIFASMQKPEGLPHISDVVLDK |
| | | ANKTPLRPLDPTRLQGINCGPDFTPSFANLGRTTLSTRGPPRGGPGGELP |
| | | RGPAGLGPRRSQQGPRKEPRKIIATVLMTEDIKLNKAEKAWKPSSKRTA |
| | | ADKDRGEEDADGSKTQDLFRRVRSIANKATPQMAQQLMKQVTQLAID |
| | | TEERLKGVIDLIFEKAISEPNFSVAYANMCRCLMALKVPTTEKPTVTVNF |
| | | RKLLLNRCQKEFEKDKDDDEVFEKKQKEMDEAATAEERGRLKEELEE |
| | | ARDIARRRSLGNIKFIGELFKLKMLTEAIMHDCVVKLLKNHDEESLECL |
| | | CRLLTTIGKDLDFEKAKPRMDQYFNQMEKIIKEKKTSSRIRFMLQDVLD |
| | | LRGSNWVPRRGDQGPKTIDQIHKEAEMEEHREHIKVQQLMAKGSDKRR |
| | | GGPPGPPISRGLPLVDDGGWNTVPISKGSRPIDTSRLTKITKPGSIDSNNQ |
| | | LFAPGGRLSWGKGSSGGSGAKPSDAASEAARPATSTLNRFSALQQAVP |
| | | TESTDNRRVVQRSSLSRERGEKAGDRGDR |
| | | LERSERGGDRGDRLDRARTPATKRSFSKEVEERSRERPSQPEGLRKAAS |
| | | LTEDRDRGRDAVKREAALPPVSPLKAALSEEELEKKSKAIIEEYLHLND |
| | | MKEAVQCVQELASPSLLFIFVRHGVESTLERSAIAREHMGQLLHQLLCA |
| | | GHLSTAQYYQGLYEILELAEDMEIDIPHVWLYLAELVTPILQEGGVPMG |
| | | ELFREITKPLRPLGKAASLLLEILGLLCKSMGPKKVGTLWREAGLSWKE |
| | | FLPEGQDIGAFVAEQKVEYTLGEESEAPGQRALPSEELNRQLEKLLKEG |
| | | SSNQRVFDWIEANLSEQQIVSNTLVRALMTAVCYSAIIFETPLRVDVAVL |
| | | KARAKLLQKYLCDEQKELQALYALQALVVTLEQPPNLLRMFFDALYD |
| | | EDVVKEDAFYSWESSKDPAEQQGKGVALK |
| | | SVTAFFKWLREAEEESDHN |
| 15 | MBP_eIF4G-dN | ATGGCCAGCAACTTCACCCAGTTCGTGCTGGTGGACAACGGCGG |
| | (623-1599, 3A) (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCAT |
| | | ATTCGCGAGCATGCAGAAGCCTGAAGGTCTGCCTCACATCAGCGAC |
| | | GTGGTGCTGGACAAGGCCAACAAGACCCCTCTTAGACCTCTGGACCC |
| | | TACCAGACTGCAGGGCATCAACTGCGGCCCTGACTTCACTCCTTCTT |
| | | TCGCAAACCTGGGCAGAACCACCCTGAGCACCAGAGGCCCTCCTAG |
| | | AGGTGGTCCCGGCGGAGAACTCCCCAGGGGTCCTGCCGGCCTGGGC |
| | | CCTAGACGCTCCCAGCAAGGTCCTAGAAAGGAGCCAAGGAAGATCA |
| | | TCGCCACCGTGCTGATGACCGAGGACATCAAGCTGAACAAAGCTGA |
| | | GAAGGCCTGGAAGCCTAGCAGCAAGAGAACCGCCGCCGACAAGGA |
| | | CAGAGGCGAGGAGGACGCCGACGGATCCAAGACCCAGGACCTGTTC |
| | | AGAAGAGTGAGAAGCATCGCGAACAAGGCGACCCCTCAGATGGCGC |
| | | AGCAGCTGATGAAGCAGGTGACGCAGCTCGCCATCGACACCGAGGA |
| | | GAGACTGAAGGGCGTGATCGACCTGATCTTTGAGAAGGCTATCTCA |
| | | GAGCCTAACTTCAGCGTGGCCTACGCCAACATGTGCCGTTGCCTGAT |
| | | GGCATTGAAGGTGCCAACCACCGAGAAGCCTACTGTGACCGTCAAT |
| | | TTCCGTAAACTGCTGCTGAACCGGTGCCAGAAAGAGTTCGAGAAGG |
| | | ATAAGGACGACGACGAGGTCTTCGAGAAGAAACAGAAAGAAATGG |
| | | ACGAGGCCGCCACCGCAGAGGAAAGGGGCCGATTAAAGGAGGAGC |
| | | TGGAGGAGGCCAGAGACATCGCCAGACGGCGTTCTCTGGGCAACAT |
| | | CAAGTTCATAGGTGAGCTGTTCAAGCTAAAGATGCTCACCGAGGCC |
| | | ATAATGCACGACTGCGTGGTGAAGCTACTGAAGAACCACGACGAAG |
| | | AAAGCCTGGAGTGCCTGTGCAGACTGCTGACCACCATCGGCAAGGA |
| | | CCTGGACTTCGAGAAGGCAAAGCCTCGAATGGACCAGTACTTCAAC |
| | | CAGATGGAGAAGATTATCAAGGAGAAGAAGACCAGCAGCAGAATC |
| | | AGATTCATGCTGCAGGACGTACTGGACCTGCGCGGAAGCAACTGGG |
| | | TGCCAAGGAGAGGGGACCAAGGACCAAAGACCATCGACCAGATCCA |
| | | CAAGGAAGCGGAGATGGAGGAGCACAGAGAGCACATAAAGGTGCA |
| | | GCAGCTTATGGCCAAGGGCAGCGACAAGCGAAGAGGCGGCCCGCCC |
| | | GGACCTCCTATCAGCAGAGGCCTTCCTCTGGTAGACGACGGCGGCTG |
| | | GAACACCGTGCCTATCTCTAAGGGCTCCAGACCTATCGACACTTCCC |
| | | GTCTTACCAAGATCACCAAGCCAGGATCTATTGACAGCAACAACCA |
| | | GCTGTTCGCCCCAGGAGGAAGACTTAGCTGGGGCAAGGGAAGTTCC |
| | | GGCGGATCCGGCGCCAAGCCTTCCGACGCCGCCAGCGAGGCTGCCA |
| | | GACCTGCCACCAGCACCTTGAACCGCTTTTCCGCTCTGCAGCAAGCT |
| | | GTGCCGACCGAGAGCACCGACAACCGGCGAGTTGTGCAGAGAAGCA |
| | | GCCTGAGCAGAGAGAGGGGCGAGAAAGCCGGCGATAGAGGTGACC |
| | | GACTGGAGAGAAGCGAGAGAGGAGGTGATAGAGGCGACCGGCTTG |
| | | ACAGAGCCAGAACCCCTGCCACAAAGCGATCGTTCAGCAAGGAGGT |
| | | CGAAGAGAGGTCCAGGGAGCGCCCTAGCCAGCCGGAAGGACTCAGA |
| | | AAGGCAGCCAGTCTAACAGAGGACCGCGACAGGGGAAGAGACGCC |
| | | GTGAAGAGGGAGGCCGCACTGCCTCCTGTGAGCCCTCTGAAGGCGG |
| | | CACTGAGCGAAGAAGAACTTGAGAAGAAGAGTAAGGCGATCATCGA |
| | | GGAGTACCTGCACCTGAACGACATGAAGGAGGCCGTGCAGTGCGTG |
| | | CAAGAGCTCGCGAGCCCATCACTGCTGTTCATCTTCGTCCGGCACGG |
| | | CGTGGAGTCCACACTGGAAAGATCTGCCATTGCTAGGGAGCATATG |
| | | GGCCAGTTGTTGCACCAATTGCTTTGCGCCGGCCACCTAAGTACTGC |
| | | CCAGTACTATCAGGGTTTATACGAGATCCTCGAACTGGCCGAAGACA |
| | | TGGAGATCGACATCCCTCACGTGTGGCTGTACCTCGCAGAACTGGTG |
| | | ACCCCTATCCTGCAGGAGGGCGGCGTTCCAATGGGTGAGTTGTTCAG |
| | | AGAAATCACAAAGCCACTGCGCCCACTGGGCAAGGCGGCTAGTCTC |
| | | CTTCTGGAGATTCTCGGCCTGCTCTGTAAGAGCATGGGTCCAAAGAA |
| | | GGTGGGCACCCTGTGGAGGGAAGCTGGACTCTCCTGGAAGGAATTC |
| | | CTCCCTGAGGGTCAGGACATCGGCGCCTTCGTGGCCGAACAGAAGG |
| | | TTGAGTACACCCTGGGAGAGGAATCGGAAGCGCCGGGTCAGCGGGC |
| | | TCTGCCGAGTGAGGAGCTCAACAGACAACTCGAGAAGCTCCTCAAG |
| | | GAGGGATCCTCAAATCAGAGAGTGTTCGACTGGATCGAGGCCAATC |
| | | TCAGCGAGCAGCAAATCGTGAGCAACACGTTGGTTCGCGCTCTCATG |
| | | ACAGCCGTGTGCTACTCAGCCATTATCTTCGAGACGCCTCTCCGCGT |
| | | GGACGTGGCAGTGCTCAAGGCCCGCGCTAAGCTGTTACAGAAGTAC |
| | | CTGTGCGACGAGCAGAAGGAACTGCAGGCCCTGTACGCCCTGCAAG |
| | | CTCTGGTCGTCACACTCGAGCAGCCTCCTAACCTGCTGAGAATGTTC |
| | | TTCGACGCATTGTACGACGAGGACGTTGTGAAGGAAGACGCTTTCTA |
| | | CAGCTGGGAGTCTAGTAAGGATCCTGCGGAACAACAAGGCAAGGGC |
| | | GTTGCTCTAAAGAGCGTGACCGCCTTCTTCAAGTGGCTGCGAGAGGC |
| | | TGAGGAAGAGTCGGACCACAAC |
| 16 | MBP_eIF4G-dN2 | MASNFTQFVLVDNGGTGDVTVAPSNFANGIAEWISSNSRSQAYKV |
| | (653-1599) (aa) | TCSVRQSSAQNRKYTIKVEVPKGAWRSYLNMELTIPIFATNSDCELIVK |
| | | AMQGLLKDGNPIPSAIAANSGIYGGGGSDPTRLQGINCGPDFTPSFANLG |
| | | RTTLSTRGPPRGGPGGELPRGPAGLGPRRSQQGPRKEPRKIIATVLMTED |
| | | IKLNKAEKAWKPSSKRTAADKDRGEEDADGSKTQDLFRRVRSILNKLT |
| | | PQMFQQLMKQVTQLAIDTEERLKGVIDLIFEKAISEPNFSVAYANMCRC |
| | | LMALKVPTTEKPTVTVNFRKLLLNRCQKEFEKDKDDDEVFEKKQKEM |
| | | DEAATAEERGRLKEELEEARDIARRRSLGNIKFIGELFKLKMLTEAIMHD |
| | | CVVKLLKNHDEESLECLCRLLTTIGKDLDFEKAKPRMDQYFNQMEKIIK |
| | | EKKTSSRIRFMLQDVLDLRGSNWVPRRGDQGPKTIDQIHKEAEMEEHR |
| | | EHIKVQQLMAKGSDKRRGGPPGPPISRGLPLVDDGGWNTVPISKGSRPI |
| | | DTSRLTKITKPGSIDSNNQLFAPGGRLSWGKGSSGGSGAKPSDAASEAA |
| | | RPATSTLNRFSALQQAVPTESTDNRRVVQRSSLSRERGEKAGDRGDRLE |
| | | RSERGGDRGDRLDRARTPATKRSFSKEVEERSRERPSQPEGLRKAASLT |
| | | EDRDRGRDAVKREAALPPVSPLKAALSEEELEKKSKAIIEEYLHLNDMK |
| | | EAVQCVQELASPSLLFIFVRHGVESTLERSAIAREHMGQLLHQLLCAGH |
| | | LSTAQYYQGLYEILELAEDMEIDIPHVWLYLAELVTPILQEGGVPMGEL |
| | | FREITKPLRPLG |
| | | KAASLLLEILGLLCKSMGPKKVGTLWREAGLSWKEFLPEGQDIGAFVA |
| | | EQKVEYTLGEESEAPGQRALPSEELNRQLEKLLKEGSSNQRVFDWIEAN |
| | | LSEQQIVSNTLVRALMTAVCYSAIIFETPLRVDVAVLKARAKLLQKYLC |
| | | DEQKELQALYALQALVVTLEQPPNLLRMFFDALYDEDVVKEDAFYSW |
| | | ESSKDPAEQQGKGVALKSVTAFFKWLREAEEESDHN |
| 17 | MBP_eIF4G-dN2 | ATGGCCAGCAACTTCACCCAGTTCGTGCTGGTGGACAACGGCGG |
| | (653-1599) (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCGA |
| | | CCCTACCAGACTGCAGGGCATCAACTGCGGCCCTGACTTCACTCCTT |
| | | CTTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTGT |
| | | TCAGAAGAGTGAGAAGCATCCTCAACAAGCTGACCCCTCAGATGTT |
| | | CCAGCAGCTGATGAAGCAGGTGACGCAGCTCGCCATCGACACCGAG |
| | | GAGAGACTGAAGGGCGTGATCGACCTGATCTTTGAGAAGGCTATCT |
| | | CAGAGCCTAACTTCAGCGTGGCCTACGCCAACATGTGCCGTTGCCTG |
| | | ATGGCATTGAAGGTGCCAACCACCGAGAAGCCTACTGTGACCGTCA |
| | | ATTTCCGTAAACTGCTGCTGAACCGGTGCCAGAAAGAGTTCGAGAA |
| | | GGATAAGGACGACGACGAGGTCTTCGAGAAGAAACAGAAAGAAAT |
| | | GGACGAGGCCGCCACCGCAGAGGAAAGGGGCCGATTAAAGGAGGA |
| | | GCTGGAGGAGGCCAGAGACATCGCCAGACGGCGTTCTCTGGGCAAC |
| | | ATCAAGTTCATAGGTGAGCTGTTCAAGCTAAAGATGCTCACCGAGGC |
| | | CATAATGCACGACTGCGTGGTGAAGCTACTGAAGAACCACGACGAA |
| | | GAAAGCCTGGAGTGCCTGTGCAGACTGCTGACCACCATCGGCAAGG |
| | | ACCTGGACTTCGAGAAGGCAAAGCCTCGAATGGACCAGTACTTCAA |
| | | CCAGATGGAGAAGATTATCAAGGAGAAGAAGACCAGCAGCAGAAT |
| | | CAGATTCATGCTGCAGGACGTACTGGACCTGCGCGGAAGCAACTGG |
| | | GTGCCAAGGAGAGGGGACCAAGGACCAAAGACCATCGACCAGATCC |
| | | ACAAGGAAGCGGAGATGGAGGAGCACAGAGAGCACATAAAGGTGC |
| | | AGCAGCTTATGGCCAAGGGCAGCGACAAGCGAAGAGGCGGCCCGCC |
| | | CGGACCTCCTATCAGCAGAGGCCTTCCTCTGGTAGACGACGGCGGCT |
| | | GGAACACCGTGCCTATCTCTAAGGGCTCCAGACCTATCGACACTTCC |
| | | CGTCTTACCAAGATCACCAAGCCAGGATCTATTGACAGCAACAACC |
| | | AGCTGTTCGCCCCAGGAGGAAGACTTAGCTGGGGCAAGGGAAGTTC |
| | | CGGCGGATCCGGCGCCAAGCCTTCCGACGCCGCCAGCGAGGCTGCC |
| | | AGACCTGCCACCAGCACCTTGAACCGCTTTTCCGCTCTGCAGCAAGC |
| | | TGTGCCGACCGAGAGCACCGACAACCGGCGAGTTGTGCAGAGAAGC |
| | | AGCCTGAGCAGAGAGAGGGGCGAGAAAGCCGGCGATAGAGGTGAC |
| | | CGACTGGAGAGAAGCGAGAGAGGAGGTGATAGAGGCGACCGGCTT |
| | | GACAGAGCCAGAACCCCTGCCACAAAGCGATCGTTCAGCAAGGAGG |
| | | TCGAAGAGAGGTCCAGGGAGCGCCCTAGCCAGCCGGAAGGACTCAG |
| | | AAAGGCAGCCAGTCTAACAGAGGACCGCGACAGGGGAAGAGACGC |
| | | CGTGAAGAGGGAGGCCGCACTGCCTCCTGTGAGCCCTCTGAAGGCG |
| | | GCACTGAGCGAAGAAGAACTTGAGAAGAAGAGTAAGGCGATCATCG |
| | | AGGAGTACCTGCACCTGAACGACATGAAGGAGGCCGTGCAGTGCGT |
| | | GCAAGAGCTCGCGAGCCCATCACTGCTGTTCATCTTCGTCCGGCACG |
| | | GCGTGGAGTCCACACTGGAAAGATCTGCCATTGCTAGGGAGCATAT |
| | | GGGCCAGTTGTTGCACCAATTGCTTTGCGCCGGCCACCTAAGTACTG |
| | | CCCAGTACTATCAGGGTTTATACGAGATCCTCGAACTGGCCGAAGAC |
| | | ATGGAGATCGACATCCCTCACGTGTGGCTGTACCTCGCAGAACTGGT |
| | | GACCCCTATCCTGCAGGAGGGCGGCGTTCCAATGGGTGAGTTGTTCA |
| | | GAGAAATCACAAAGCCACTGCGCCCACTGGGCAAGGCGGCTAGTCT |
| | | CCTTCTGGAGATTCTCGGCCTGCTCTGTAAGAGCATGGGTCCAAAGA |
| | | AGGTGGGCACCCTGTGGAGGGAAGCTGGACTCTCCTGGAAGGAATT |
| | | CCTCCCTGAGGGTCAGGACATCGGCGCCTTCGTGGCCGAACAGAAG |
| | | GTTGAGTACACCCTGGGAGAGGAATCGGAAGCGCCGGGTCAGCGGG |
| | | CTCTGCCGAGTGAGGAGCTCAACAGACAACTCGAGAAGCTCCTCAA |
| | | GGAGGGATCCTCAAATCAGAGAGTGTTCGACTGGATCGAGGCCAAT |
| | | CTCAGCGAGCAGCAAATCGTGAGCAACACGTTGGTTCGCGCTCTCAT |
| | | GACAGCCGTGTGCTACTCAGCCATTATCTTCGAGACGCCTCTCCGCG |
| | | TGGACGTGGCAGTGCTCAAGGCCCGCGCTAAGCTGTTACAGAAGTA |
| | | CCTGTGCGACGAGCAGAAGGAACTGCAGGCCCTGTACGCCCTGCAA |
| | | GCTCTGGTCGTCACACTCGAGCAGCCTCCTAACCTGCTGAGAATGTT |
| | | CTTCGACGCATTGTACGACGAGGACGTTGTGAAGGAAGACGCTTTCT |
| | | ACAGCTGGGAGTCTAGTAAGGATCCTGCGGAACAACAAGGCAAGGG |
| | | CGTTGCTCTAAAGAGCGTGACCGCCTTCTTCAAGTGGCTGCGAGAGG |
| | | CTGAGGAAGAGTCGGACCACAAC |
| 18 | MBP_eIF4G-dN3 | MASNFTQFVLVDNGGTGDVTVAPSNFANGIAEWISSNSRSQAYKV |
| | (653-1451) (aa) | TCSVRQSSAQNRKYTIKVEVPKGAWRSYLNMELTIPIFATNSDCELIVK |
| | | AMQGLLKDGNPIPSAIAANSGIYGGGGSDPTRLQGINCGPDFTPSFANLG |
| | | RTTLSTRGPPRGGPGGELPRGPAGLGPRRSQQGPRKEPRKIIATVLMTED |
| | | IKLNKAEKAWKPSSKRTAADKDRGEEDADGSKTQDLFRRVRSILNKLT |
| | | PQMFQQLMKQVTQLAIDTEERLKGVIDLIFEKAISEPNFSVAYANMCRC |
| | | LMALKVPTTEKPTVTVNFRKLLLNRCQKEFEKDKDDDEVFEKKQKEM |
| | | DEAATAEERGRLKEELEEARDIARRRSLGNIKFIGELFKLKMLTEAIMHD |
| | | CVVKLLKNHDEESLECLCRLLTTIGKDLDFEKAKPRMDQYFNQMEKIIK |
| | | EKKTSSRIRFMLQDVLDLRGSNWVPRRGDQGPKTIDQIHKEAEMEEHR |
| | | EHIKVQQLMAKGSDKRRGGPPGPPISRGLPLVDDGGWNTVPISKGSRPI |
| | | DTSRLTKITKPGSIDSNNQLFAPGGRLSWGKGSSGGSGAKPSDAASEAA |
| | | RPATSTLNRFSALQQAVPTESTDNRRVVQRSSLSRERGEKAGDRGDRLE |
| | | RSERGGDRGDRLDRARTPATKRSFSKEVEERSRERPSQPEGLRKAASLT |
| | | EDRDRGRDAVKREAALPPVSPLKAALSEEELEKKSKAIIEEYLHLNDMK |
| | | EAVQCVQELASPSLLFIFVRHGVESTLERSAIAREHMGQLLHQLLCAGH |
| | | LSTAQYYQGLYEILELAEDMEIDIPHVWLYLAELVTPILQEGGVPMGEL |
| | | FREITKPLRPLG |
| | | KAASLLLEILGLLCKSMGPKKVGTLWREAGLSWKEFLPEGQDIGAFVA |
| | | EQKVEYTLGEES EAPGQRALPSEELNRQLEKLL |
| 19 | MBP_eIF4G-dN3 | ATGGCCAGCAACTTCACCCAGTTCGTGCTGGTGGACAACGGCGG |
| | (653-1451) (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCGA |
| | | CCCTACCAGACTGCAGGGCATCAACTGCGGCCCTGACTTCACTCCTT |
| | | CTTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTGT |
| | | TCAGAAGAGTGAGAAGCATCCTCAACAAGCTGACCCCTCAGATGTT |
| | | CCAGCAGCTGATGAAGCAGGTGACGCAGCTCGCCATCGACACCGAG |
| | | GAGAGACTGAAGGGCGTGATCGACCTGATCTTTGAGAAGGCTATCT |
| | | CAGAGCCTAACTTCAGCGTGGCCTACGCCAACATGTGCCGTTGCCTG |
| | | ATGGCATTGAAGGTGCCAACCACCGAGAAGCCTACTGTGACCGTCA |
| | | ATTTCCGTAAACTGCTGCTGAACCGGTGCCAGAAAGAGTTCGAGAA |
| | | GGATAAGGACGACGACGAGGTCTTCGAGAAGAAACAGAAAGAAAT |
| | | GGACGAGGCCGCCACCGCAGAGGAAAGGGGCCGATTAAAGGAGGA |
| | | GCTGGAGGAGGCCAGAGACATCGCCAGACGGCGTTCTCTGGGCAAC |
| | | ATCAAGTTCATAGGTGAGCTGTTCAAGCTAAAGATGCTCACCGAGGC |
| | | CATAATGCACGACTGCGTGGTGAAGCTACTGAAGAACCACGACGAA |
| | | GAAAGCCTGGAGTGCCTGTGCAGACTGCTGACCACCATCGGCAAGG |
| | | ACCTGGACTTCGAGAAGGCAAAGCCTCGAATGGACCAGTACTTCAA |
| | | CCAGATGGAGAAGATTATCAAGGAGAAGAAGACCAGCAGCAGAAT |
| | | CAGATTCATGCTGCAGGACGTACTGGACCTGCGCGGAAGCAACTGG |
| | | GTGCCAAGGAGAGGGGACCAAGGACCAAAGACCATCGACCAGATCC |
| | | ACAAGGAAGCGGAGATGGAGGAGCACAGAGAGCACATAAAGGTGC |
| | | AGCAGCTTATGGCCAAGGGCAGCGACAAGCGAAGAGGCGGCCCGCC |
| | | CGGACCTCCTATCAGCAGAGGCCTTCCTCTGGTAGACGACGGCGGCT |
| | | GGAACACCGTGCCTATCTCTAAGGGCTCCAGACCTATCGACACTTCC |
| | | CGTCTTACCAAGATCACCAAGCCAGGATCTATTGACAGCAACAACC |
| | | AGCTGTTCGCCCCAGGAGGAAGACTTAGCTGGGGCAAGGGAAGTTC |
| | | CGGCGGATCCGGCGCCAAGCCTTCCGACGCCGCCAGCGAGGCTGCC |
| | | AGACCTGCCACCAGCACCTTGAACCGCTTTTCCGCTCTGCAGCAAGC |
| | | TGTGCCGACCGAGAGCACCGACAACCGGCGAGTTGTGCAGAGAAGC |
| | | AGCCTGAGCAGAGAGAGGGGCGAGAAAGCCGGCGATAGAGGTGAC |
| | | CGACTGGAGAGAAGCGAGAGAGGAGGTGATAGAGGCGACCGGCTT |
| | | GACAGAGCCAGAACCCCTGCCACAAAGCGATCGTTCAGCAAGGAGG |
| | | TCGAAGAGAGGTCCAGGGAGCGCCCTAGCCAGCCGGAAGGACTCAG |
| | | AAAGGCAGCCAGTCTAACAGAGGACCGCGACAGGGGAAGAGACGC |
| | | CGTGAAGAGGGAGGCCGCACTGCCTCCTGTGAGCCCTCTGAAGGCG |
| | | GCACTGAGCGAAGAAGAACTTGAGAAGAAGAGTAAGGCGATCATCG |
| | | AGGAGTACCTGCACCTGAACGACATGAAGGAGGCCGTGCAGTGCGT |
| | | GCAAGAGCTCGCGAGCCCATCACTGCTGTTCATCTTCGTCCGGCACG |
| | | GCGTGGAGTCCACACTGGAAAGATCTGCCATTGCTAGGGAGCATAT |
| | | GGGCCAGTTGTTGCACCAATTGCTTTGCGCCGGCCACCTAAGTACTG |
| | | CCCAGTACTATCAGGGTTTATACGAGATCCTCGAACTGGCCGAAGAC |
| | | ATGGAGATCGACATCCCTCACGTGTGGCTGTACCTCGCAGAACTGGT |
| | | GACCCCTATCCTGCAGGAGGGCGGCGTTCCAATGGGTGAGTTGTTCA |
| | | GAGAAATCACAAAGCCACTGCGCCCACTGGGCAAGGCGGCTAGTCT |
| | | CCTTCTGGAGATTCTCGGCCTGCTCTGTAAGAGCATGGGTCCAAAGA |
| | | AGGTGGGCACCCTGTGGAGGGAAGCTGGACTCTCCTGGAAGGAATT |
| | | CCTCCCTGAGGGTCAGGACATCGGCGCCTTCGTGGCCGAACAGAAG |
| | | GTTGAGTACACCCTGGGAGAGGAATCGGAAGCGCCGGGTCAGCGGG |
| | | CTCTGCCGAGTGAGGAGCTCAACAGACAACTCGAGAAGCTCCTC |
| 20 | MBP_eIF4G-mid1 | MASNFTQFVLVDNGGTGDVTVAPSNFANGIAEWISSNSRSQAYKV |
| | (674-1079) (aa) | TCSVRQSSAQNRKYTIKVEVPKGAWRSYLNMELTIPIFATNSDCELIVK |
| | | AMQGLLKDGNPIPSAIAANSGIYGGGGSGRTTLSTRGPPRGGPGGELPR |
| | | GPAGLGPRRSQQGPRKEPRKIIATVLMTEDIKLNKAEKAWKPSSKRTAA |
| | | DKDRGEEDADGSKTQDLFRRVRSILNKLTPQMFQQLMKQVTQLAIDTE |
| | | ERLKGVIDLIFEKAISEPNFSVAYANMCRCLMALKVPTTEKPTVTVNFR |
| | | KLLLNRCQKEFEKDKDDDEVFEKKQKEMDEAATAEERGRLKEELEEA |
| | | RDIARRRSLGNIKFIGELFKLKMLTEAIMHDCVVKLLKNHDEESLECLC |
| | | RLLTTIGKDLDFEKAKPRMDQYFNQMEKIIKEKKTSSRIRFMLQDVLDL |
| | | RGSNWVPRRGDQGPKTIDQIHKEAEMEEHREHIKVQQLMAKGSDKR |
| | | RGGPPGPPISRGLPLVDDGGWNTVPISKGSRPIDTSRLTKITKPGSID |
| 21 | MBP_eIF4G-mid1 | ATGGCCAGCAACTTCACCCAGTTCGTGCTGGTGGACAACGGCGG |
| | (674-1079) (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCGG |
| | | CAGAACCACCCTGAGCACCAGAGGCCCTCCTAGAGGTGGTCCCGGC |
| | | GGAGAACTCCCCAGGGGTCCTGCCGGCCTGGGCCCTAGACGCTCCC |
| | | AGCAAGGTCCTAGAAAGGAGCCAAGGAAGATCATCGCCACCGTGCT |
| | | GATGACCGAGGACATCAAGCTGAACAAAGCTGAGAAGGCCTGGAAG |
| | | CCTAGCAGCAAGAGAACCGCCGCCGACAAGGACAGAGGCGAGGAG |
| | | GACGCCGACGGATCCAAGACCCAGGACCTGTTCAGAAGAGTGAGAA |
| | | GCATCCTCAACAAGCTGACCCCTCAGATGTTCCAGCAGCTGATGAAG |
| | | CAGGTGACGCAGCTCGCCATCGACACCGAGGAGAGACTGAAGGGCG |
| | | TGATCGACCTGATCTTTGAGAAGGCTATCTCAGAGCCTAACTTCAGC |
| | | GTGGCCTACGCCAACATGTGCCGTTGCCTGATGGCATTGAAGGTGCC |
| | | AACCACCGAGAAGCCTACTGTGACCGTCAATTTCCGTAAACTGCTGC |
| | | TGAACCGGTGCCAGAAAGAGTTCGAGAAGGATAAGGACGACGACG |
| | | AGGTCTTCGAGAAGAAACAGAAAGAAATGGACGAGGCCGCCACCGC |
| | | AGAGGAAAGGGGCCGATTAAAGGAGGAGCTGGAGGAGGCCAGAGA |
| | | CATCGCCAGACGGCGTTCTCTGGGCAACATCAAGTTCATAGGTGAGC |
| | | TGTTCAAGCTAAAGATGCTCACCGAGGCCATAATGCACGACTGCGTG |
| | | GTGAAGCTACTGAAGAACCACGACGAAGAAAGCCTGGAGTGCCTGT |
| | | GCAGACTGCTGACCACCATCGGCAAGGACCTGGACTTCGAGAAGGC |
| | | AAAGCCTCGAATGGACCAGTACTTCAACCAGATGGAGAAGATTATC |
| | | AAGGAGAAGAAGACCAGCAGCAGAATCAGATTCATGCTGCAGGACG |
| | | TACTGGACCTGCGCGGAAGCAACTGGGTGCCAAGGAGAGGGGACCA |
| | | AGGACCAAAGACCATCGACCAGATCCACAAGGAAGCGGAGATGGA |
| | | GGAGCACAGAGAGCACATAAAGGTGCAGCAGCTTATGGCCAAGGGC |
| | | AGCGACAAGCGAAGAGGCGGCCCGCCCGGACCTCCTATCAGCAGAG |
| | | GCCTTCCTCTGGTAGACGACGGCGGCTGGAACACCGTGCCTATCTCT |
| | | AAGGGCTCCAGACCTATCGACACTTCCCGTCTTACCAAGATCACCAA |
| | | GCCAGGATCTATTGAC |
| 22 | MBP_eIF4G-mid2 | MASNFTQFVLVDNGGTGDVTVAPSNFANGIAEWISSNSRSQAYKV |
| | (653-1130) (aa) | TCSVRQSSAQNRKYTIKVEVPKGAWRSYLNMELTIPIFATNSDCELIVK |
| | | AMQGLLKDGNPIPSAIAANSGIYGGGGSDPTRLQGINCGPDFTPSFANLG |
| | | RTTLSTRGPPRGGPGGELPRGPAGLGPRRSQQGPRKEPRKIIATVLMTED |
| | | IKLNKAEKAWKPSSKRTAADKDRGEEDADGSKTQDLFRRVRSILNKLT |
| | | PQMFQQLMKQVTQLAIDTEERLKGVIDLIFEKAISEPNFSVAYANMCRC |
| | | LMALKVPTTEKPTVTVNFRKLLLNRCQKEFEKDKDDDEVFEKKQKEM |
| | | DEAATAEERGRLKEELEEARDIARRRSLGNIKFIGELFKLKMLTEAIMHD |
| | | CVVKLLKNHDEESLECLCRLLTTIGKDLDFEKAKPRMDQYFNQMEKIIK |
| | | EKKTSSRIRFMLQDVLDLRGSNWVPRRGDQGPKTIDQIHKEAEMEEHR |
| | | EHIKVQQLMAKGSDKRRGGPPGPPISRGLPLVDDGGWNTVPISKGSRPI |
| | | DTSRLTKITKPGSIDSNNQLFAPGGRLSWGKGSSGGSGAKPSDAASEAA |
| | | RPATSTLNRFSALQQAV |
| 23 | MBP_eIF4G-mid2 | ATGGCCAGCAACTTCACCCAGTTCGTGCTGGTGGACAACGGCGG |
| | (653-1130) (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCGA |
| | | CCCTACCAGACTGCAGGGCATCAACTGCGGCCCTGACTTCACTCCTT |
| | | CTTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTGT |
| | | TCAGAAGAGTGAGAAGCATCCTCAACAAGCTGACCCCTCAGATGTT |
| | | CCAGCAGCTGATGAAGCAGGTGACGCAGCTCGCCATCGACACCGAG |
| | | GAGAGACTGAAGGGCGTGATCGACCTGATCTTTGAGAAGGCTATCT |
| | | CAGAGCCTAACTTCAGCGTGGCCTACGCCAACATGTGCCGTTGCCTG |
| | | ATGGCATTGAAGGTGCCAACCACCGAGAAGCCTACTGTGACCGTCA |
| | | ATTTCCGTAAACTGCTGCTGAACCGGTGCCAGAAAGAGTTCGAGAA |
| | | GGATAAGGACGACGACGAGGTCTTCGAGAAGAAACAGAAAGAAAT |
| | | GGACGAGGCCGCCACCGCAGAGGAAAGGGGCCGATTAAAGGAGGA |
| | | GCTGGAGGAGGCCAGAGACATCGCCAGACGGCGTTCTCTGGGCAAC |
| | | ATCAAGTTCATAGGTGAGCTGTTCAAGCTAAAGATGCTCACCGAGGC |
| | | CATAATGCACGACTGCGTGGTGAAGCTACTGAAGAACCACGACGAA |
| | | GAAAGCCTGGAGTGCCTGTGCAGACTGCTGACCACCATCGGCAAGG |
| | | ACCTGGACTTCGAGAAGGCAAAGCCTCGAATGGACCAGTACTTCAA |
| | | CCAGATGGAGAAGATTATCAAGGAGAAGAAGACCAGCAGCAGAAT |
| | | CAGATTCATGCTGCAGGACGTACTGGACCTGCGCGGAAGCAACTGG |
| | | GTGCCAAGGAGAGGGGACCAAGGACCAAAGACCATCGACCAGATCC |
| | | ACAAGGAAGCGGAGATGGAGGAGCACAGAGAGCACATAAAGGTGC |
| | | AGCAGCTTATGGCCAAGGGCAGCGACAAGCGAAGAGGCGGCCCGCC |
| | | CGGACCTCCTATCAGCAGAGGCCTTCCTCTGGTAGACGACGGCGGCT |
| | | GGAACACCGTGCCTATCTCTAAGGGCTCCAGACCTATCGACACTTCC |
| | | CGTCTTACCAAGATCACCAAGCCAGGATCTATTGACAGCAACAACC |
| | | AGCTGTTCGCCCCAGGAGGAAGACTTAGCTGGGGCAAGGGAAGTTC |
| | | CGGCGGATCCGGCGCCAAGCCTTCCGACGCCGCCAGCGAGGCTGCC |
| | | AGACCTGCCACCAGCACCTTGAACCGCTTTTCCGCTCTGCAGCAAGC |
| | | TGTG |
| 24 | MBP_eIF4G-mid3 | MASNFTQFVLVDNGGTGDVTVAPSNFANGIAEWISSNSRSQAYKV |
| | (712-1130) (aa) | TCSVRQSSAQNRKYTIKVEVPKGAWRSYLNMELTIPIFATNSDCELIVK |
| | | AMQGLLKDGNPIPSAIAANSGIYGGGGSPRKIIATVLMTEDIKLNKAEKA |
| | | WKPSSKRTAADKDRGEEDADGSKTQDLFRRVRSILNKLTPQMFQQLM |
| | | KQVTQLAIDTEERLKGVIDLIFEKAISEPNFSVAYANMCRCLMALKVPT |
| | | TEKPTVTVNFRKLLLNRCQKEFEKDKDDDEVFEKKQKEMDEAATAEER |
| | | GRLKEELEEARDIARRRSLGNIKFIGELFKLKMLTEAIMHDCVVKLLKN |
| | | HDEESLECLCRLLTTIGKDLDFEKAKPRMDQYFNQMEKIIKEKKTSSRIR |
| | | FMLQDVLDLRGSNWVPRRGDQGPKTIDQIHKEAEMEEHREHIKVQQL |
| | | MAKGSDKRRGGPPGPPISRGLPLVDDGGWNTVPISKGSRPIDTSRLTKIT |
| | | KPGSIDSNNQLFAPGGRLSWGKGSSGGSGAKPSDAASEAARPATSTLNR |
| | | FSALQQAV |
| 25 | MBP_eIF4G-mid3 | ATGGCCAGCAACTTCACCCAGTTCGTGCTGGTGGACAACGGCGG |
| | (712-1130) (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCCC |
| | | AAGGAAGATCATCGCCACCGTGCTGATGACCGAGGACATCAAGCTG |
| | | AACAAAGCTGAGAAGGCCTGGAAGCCTAGCAGCAAGAGAACCGCC |
| | | GCCGACAAGGACAGAGGCGAGGAGGACGCCGACGGATCCAAGACC |
| | | CAGGACCTGTTCAGAAGAGTGAGAAGCATCCTCAACAAGCTGACCC |
| | | CTCAGATGTTCCAGCAGCTGATGAAGCAGGTGACGCAGCTCGCCATC |
| | | GACACCGAGGAGAGACTGAAGGGCGTGATCGACCTGATCTTTGAGA |
| | | AGGCTATCTCAGAGCCTAACTTCAGCGTGGCCTACGCCAACATGTGC |
| | | CGTTGCCTGATGGCATTGAAGGTGCCAACCACCGAGAAGCCTACTGT |
| | | GACCGTCAATTTCCGTAAACTGCTGCTGAACCGGTGCCAGAAAGAGT |
| | | TCGAGAAGGATAAGGACGACGACGAGGTCTTCGAGAAGAAACAGA |
| | | AAGAAATGGACGAGGCCGCCACCGCAGAGGAAAGGGGCCGATTAA |
| | | AGGAGGAGCTGGAGGAGGCCAGAGACATCGCCAGACGGCGTTCTCT |
| | | GGGCAACATCAAGTTCATAGGTGAGCTGTTCAAGCTAAAGATGCTC |
| | | ACCGAGGCCATAATGCACGACTGCGTGGTGAAGCTACTGAAGAACC |
| | | ACGACGAAGAAAGCCTGGAGTGCCTGTGCAGACTGCTGACCACCAT |
| | | CGGCAAGGACCTGGACTTCGAGAAGGCAAAGCCTCGAATGGACCAG |
| | | TACTTCAACCAGATGGAGAAGATTATCAAGGAGAAGAAGACCAGCA |
| | | GCAGAATCAGATTCATGCTGCAGGACGTACTGGACCTGCGCGGAAG |
| | | CAACTGGGTGCCAAGGAGAGGGGACCAAGGACCAAAGACCATCGA |
| | | CCAGATCCACAAGGAAGCGGAGATGGAGGAGCACAGAGAGCACAT |
| | | AAAGGTGCAGCAGCTTATGGCCAAGGGCAGCGACAAGCGAAGAGG |
| | | CGGCCCGCCCGGACCTCCTATCAGCAGAGGCCTTCCTCTGGTAGACG |
| | | ACGGCGGCTGGAACACCGTGCCTATCTCTAAGGGCTCCAGACCTATC |
| | | GACACTTCCCGTCTTACCAAGATCACCAAGCCAGGATCTATTGACAG |
| | | CAACAACCAGCTGTTCGCCCCAGGAGGAAGACTTAGCTGGGGCAAG |
| | | GGAAGTTCCGGCGGATCCGGCGCCAAGCCTTCCGACGCCGCCAGCG |
| | | AGGCTGCCAGACCTGCCACCAGCACCTTGAACCGCTTTTCCGCTCTG |
| | | CAGCAAGCTGTG |
| 26 | MBP_eIF4G-C1 | MASNFTQFVLVDNGGTGDVTVAPSNFANGIAEWISSNSRSQAYKV |
| | (1080-1599) (aa) | TCSVRQSSAQNRKYTIKVEVPKGAWRSYLNMELTIPIFATNSDCELIVK |
| | | AMQGLLKDGNPIPSAIAANSGIYGGGGSSNNQLFAPGGRLSWGKGSSG |
| | | GSGAKPSDAASEAARPATSTLNRFSALQQAVPTESTDNRRVVQRSSLSR |
| | | ERGEKAGDRGDRLERSERGGDRGDRLDRARTPATKRSFSKEVEERSRE |
| | | RPSQPEGLRKAASLTEDRDRGRDAVKREAALPPVSPLKAALSEEELEKK |
| | | SKAIIEEYLHLNDMKEAVQCVQELASPSLLFIFVRHGVESTLERSAIARE |
| | | HMGQLLHQLLCAGHLSTAQYYQGLYEILELAEDMEIDIPHVWLYLAEL |
| | | VTPILQEGGVPMGELFREITKPLRPLGKAASLLLEILGLLCKSMGPKKVG |
| | | TLWREAGLSWKEFLPEGQDIGAFVAEQKVEYTLGEESEAPGQRALPSEE |
| | | LNRQLEKLLKEGSSNQRVFDWIEANLSEQQIVSNTLVRALMTAVCYSAI |
| | | IFETPLRVDVAVLKARAKLLQKYLCDEQKELQALYALQALVVTLEQPP |
| | | NLLRMFFDALYDEDVVKEDAFYSWESSKDPAEQQGKGVALKSVTAFF |
| | | KWLREAEEESDHN |
| 27 | MBP_eIF4G-C1 | ATGGCCAGCAACTTCACCCAGTTCGTGCTGGTGGACAACGGCGG |
| | (1080-1599) (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCAG |
| | | CAACAACCAGCTGTTCGCCCCAGGAGGAAGACTTAGCTGGGGCAAG |
| | | GGAAGTTCCGGCGGATCCGGCGCCAAGCCTTCCGACGCCGCCAGCG |
| | | AGGCTGCCAGACCTGCCACCAGCACCTTGAACCGCTTTTCCGCTCTG |
| | | CAGCAAGCTGTGCCGACCGAGAGCACCGACAACCGGCGAGTTGTGC |
| | | AGAGAAGCAGCCTGAGCAGAGAGAGGGGCGAGAAAGCCGGCGATA |
| | | GAGGTGACCGACTGGAGAGAAGCGAGAGAGGAGGTGATAGAGGCG |
| | | ACCGGCTTGACAGAGCCAGAACCCCTGCCACAAAGCGATCGTTCAG |
| | | CAAGGAGGTCGAAGAGAGGTCCAGGGAGCGCCCTAGCCAGCCGGA |
| | | AGGACTCAGAAAGGCAGCCAGTCTAACAGAGGACCGCGACAGGGG |
| | | AAGAGACGCCGTGAAGAGGGAGGCCGCACTGCCTCCTGTGAGCCCT |
| | | CTGAAGGCGGCACTGAGCGAAGAAGAACTTGAGAAGAAGAGTAAG |
| | | GCGATCATCGAGGAGTACCTGCACCTGAACGACATGAAGGAGGCCG |
| | | TGCAGTGCGTGCAAGAGCTCGCGAGCCCATCACTGCTGTTCATCTTC |
| | | GTCCGGCACGGCGTGGAGTCCACACTGGAAAGATCTGCCATTGCTA |
| | | GGGAGCATATGGGCCAGTTGTTGCACCAATTGCTTTGCGCCGGCCAC |
| | | CTAAGTACTGCCCAGTACTATCAGGGTTTATACGAGATCCTCGAACT |
| | | GGCCGAAGACATGGAGATCGACATCCCTCACGTGTGGCTGTACCTCG |
| | | CAGAACTGGTGACCCCTATCCTGCAGGAGGGCGGCGTTCCAATGGG |
| | | TGAGTTGTTCAGAGAAATCACAAAGCCACTGCGCCCACTGGGCAAG |
| | | GCGGCTAGTCTCCTTCTGGAGATTCTCGGCCTGCTCTGTAAGAGCAT |
| | | GGGTCCAAAGAAGGTGGGCACCCTGTGGAGGGAAGCTGGACTCTCC |
| | | TGGAAGGAATTCCTCCCTGAGGGTCAGGACATCGGCGCCTTCGTGGC |
| | | CGAACAGAAGGTTGAGTACACCCTGGGAGAGGAATCGGAAGCGCCG |
| | | GGTCAGCGGGCTCTGCCGAGTGAGGAGCTCAACAGACAACTCGAGA |
| | | AGCTCCTCAAGGAGGGATCCTCAAATCAGAGAGTGTTCGACTGGAT |
| | | CGAGGCCAATCTCAGCGAGCAGCAAATCGTGAGCAACACGTTGGTT |
| | | CGCGCTCTCATGACAGCCGTGTGCTACTCAGCCATTATCTTCGAGAC |
| | | GCCTCTCCGCGTGGACGTGGCAGTGCTCAAGGCCCGCGCTAAGCTGT |
| | | TACAGAAGTACCTGTGCGACGAGCAGAAGGAACTGCAGGCCCTGTA |
| | | CGCCCTGCAAGCTCTGGTCGTCACACTCGAGCAGCCTCCTAACCTGC |
| | | TGAGAATGTTCTTCGACGCATTGTACGACGAGGACGTTGTGAAGGA |
| | | AGACGCTTTCTACAGCTGGGAGTCTAGTAAGGATCCTGCGGAACAA |
| | | CAAGGCAAGGGCGTTGCTCTAAAGAGCGTGACCGCCTTCTTCAAGTG |
| | | GCTGCGAGAGGCTGAGGAAGAGTCGGACCACAAC |
| 28 | MBP_eIF4G-C2 | MASNFTQFVLVDNGGTGDVTVAPSNFANGIAEWISSNSRSQAYKV |
| | (1080-1451) (aa) | TCSVRQSSAQNRKYTIKVEVPKGAWRSYLNMELTIPIFATNSDCELIVK |
| | | AMQGLLKDGNPIPSAIAANSGIYGGGGSSNNQLFAPGGRLSWGKGSSG |
| | | GSGAKPSDAASEAARPATSTLNRFSALQQAVPTESTDNRRVVQRSSLSR |
| | | ERGEKAGDRGDRLERSERGGDRGDRLDRARTPATKRSFSKEVEERSRE |
| | | RPSQPEGLRKAASLTEDRDRGRDAVKREAALPPVSPLKAALSEEELEKK |
| | | SKAIIEEYLHLNDMKEAVQCVQELASPSLLFIFVRHGVESTLERSAIARE |
| | | HMGQLLHQLLCAGHLSTAQYYQGLYEILELAEDMEIDIPHVWLYLAEL |
| | | VTPILQEGGVPMGELFREITKPLRPLGKAASLLLEILGLLCKSMGPKKVG |
| | | TLWREAGLSWKEFLPEGQDIGAFVAEQKVEYTLGEESEAPGQRALPSEE |
| | | LNRQLEKLL |
| 29 | MBP_eIF4G-C2 | ATGGCCAGCAACTTCACCCAGTTCGTGCTGGTGGACAACGGCGG |
| | (1080-1451) (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCAG |
| | | CAACAACCAGCTGTTCGCCCCAGGAGGAAGACTTAGCTGGGGCAAG |
| | | GGAAGTTCCGGCGGATCCGGCGCCAAGCCTTCCGACGCCGCCAGCG |
| | | AGGCTGCCAGACCTGCCACCAGCACCTTGAACCGCTTTTCCGCTCTG |
| | | CAGCAAGCTGTGCCGACCGAGAGCACCGACAACCGGCGAGTTGTGC |
| | | AGAGAAGCAGCCTGAGCAGAGAGAGGGGCGAGAAAGCCGGCGATA |
| | | GAGGTGACCGACTGGAGAGAAGCGAGAGAGGAGGTGATAGAGGCG |
| | | ACCGGCTTGACAGAGCCAGAACCCCTGCCACAAAGCGATCGTTCAG |
| | | CAAGGAGGTCGAAGAGAGGTCCAGGGAGCGCCCTAGCCAGCCGGA |
| | | AGGACTCAGAAAGGCAGCCAGTCTAACAGAGGACCGCGACAGGGG |
| | | AAGAGACGCCGTGAAGAGGGAGGCCGCACTGCCTCCTGTGAGCCCT |
| | | CTGAAGGCGGCACTGAGCGAAGAAGAACTTGAGAAGAAGAGTAAG |
| | | GCGATCATCGAGGAGTACCTGCACCTGAACGACATGAAGGAGGCCG |
| | | TGCAGTGCGTGCAAGAGCTCGCGAGCCCATCACTGCTGTTCATCTTC |
| | | GTCCGGCACGGCGTGGAGTCCACACTGGAAAGATCTGCCATTGCTA |
| | | GGGAGCATATGGGCCAGTTGTTGCACCAATTGCTTTGCGCCGGCCAC |
| | | CTAAGTACTGCCCAGTACTATCAGGGTTTATACGAGATCCTCGAACT |
| | | GGCCGAAGACATGGAGATCGACATCCCTCACGTGTGGCTGTACCTCG |
| | | CAGAACTGGTGACCCCTATCCTGCAGGAGGGCGGCGTTCCAATGGG |
| | | TGAGTTGTTCAGAGAAATCACAAAGCCACTGCGCCCACTGGGCAAG |
| | | GCGGCTAGTCTCCTTCTGGAGATTCTCGGCCTGCTCTGTAAGAGCAT |
| | | GGGTCCAAAGAAGGTGGGCACCCTGTGGAGGGAAGCTGGACTCTCC |
| | | TGGAAGGAATTCCTCCCTGAGGGTCAGGACATCGGCGCCTTCGTGGC |
| | | CGAACAGAAGGTTGAGTACACCCTGGGAGAGGAATCGGAAGCGCCG |
| | | GGTCAGCGGGCTCTGCCGAGTGAGGAGCTCAACAGACAACTCGAGA |
| | | AGCTCCTC |
| 30 | MBP-CTIF (aa) | MASNFTQFVLVDNGGTGDVTVAPSNFANGIAEWISSNSRSQAYKV |
| | | TCSVRQSSAQNRKYTIKVEVPKGAWRSYLNMELTIPIFATNSDCELIVK |
| | | AMQGLLKDGNPIPSAIAANSGIYGGGGSENSSAASASSEAGSSRSQEIEE |
| | | LERFIDSYVLEYQVQGLLADKTEGDGESERTQSHISQWTADCSEPLDSS |
| | | CSFSRGRAPPQQNGSKDNSLDMLGTDIWAANTFDSFSGATWDLQPEKL |
| | | DFTQFHRKVRHTPKQPLPHIDREGCGKGKLEDGDGINLNDIEKVLPAW |
| | | QGYHPMPHEVEIAHTKKLFRRRRNDRRRQQRPPGGNKPQQHGDHQPG |
| | | SAKHNRDHQKSYQGGSAPHPSGRPTHHGYSQNRRWHHGNMKHPPGD |
| | | KGEAGAHRNAKETMTIENPKLEDTAGDTGHSSLEAPRSPDTLAPVASER |
| | | LPPQQSGGPEVETKRKDSILPERIGERPKITLLQSSKDRLRRRLKEKDEV |
| | | AVETTTPQQNKMDKLIEILNSMRNNSSDVDTKLTTFMEEAQNSTNSEE |
| | | MLGEIVRTIYQKAVSDRSFAFTAAKLCDKMALFMVEGTKFRSLLLNML |
| | | QKDFTVREELQQQDVERWLGFITFLCEVFGTMRSSTGEPFRVLVCPIYT |
| | | CLRELLQSQDVKEDAVLCCSMELQSTGRLLEEQLPEMMTELLASARDK |
| | | MLCPSESMLTRSLLLEVIELHANSWNPLTPPITQYYNRTIQKLTA |
| 31 | MBP-CTIF (nt) | ATGGCCAGCAACTTCACCCAGTTCGTGCTGGTGGACAACGGCGG |
| | | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCGA |
| | | GAACAGTAGCGCCGCTAGCGCGAGCAGCGAAGCCGGGAGCAGCCG |
| | | GTCTCAGGAGATCGAGGAGCTGGAGCGGTTCATCGACAGCTACGTG |
| | | CTGGAGTACCAGGTGCAGGGCCTGCTGGCCGACAAGACCGAGGGCG |
| | | ACGGCGAGAGCGAGCGGACCCAGAGCCACATCAGCCAGTGGACCGC |
| | | CGACTGCAGCGAGCCCCTGGACTCTAGCTGCTCATTCAGTCGTGGAC |
| | | GGGCCCCTCCACAGCAGAACGGCAGCAAGGACAACAGCCTGGACAT |
| | | GCTGGGCACCGACATCTGGGCCGCCAACACCTTCGACAGCTTCAGCG |
| | | GCGCCACCTGGGATCTGCAGCCCGAGAAGCTGGACTTTACCCAGTTC |
| | | CACCGGAAGGTGCGGCACACTCCCAAGCAGCCCCTGCCCCACATCG |
| | | ATCGGGAGGGCTGCGGCAAGGGCAAGCTGGAAGACGGCGACGGCA |
| | | TCAACCTGAACGACATCGAGAAGGTGCTGCCTGCCTGGCAGGGCTA |
| | | CCACCCCATGCCCCACGAGGTGGAGATCGCCCACACCAAGAAGCTG |
| | | TTCCGGCGACGACGCAACGACCGGCGTAGGCAGCAACGGCCGCCTG |
| | | GAGGGAACAAGCCCCAGCAGCACGGAGACCACCAGCCCGGTAGCGC |
| | | CAAGCACAACCGGGACCACCAGAAGAGCTACCAGGGCGGAAGCGC |
| | | ACCACACCCCTCGGGCAGACCCACCCACCACGGCTACAGCCAGAAC |
| | | CGGCGGTGGCATCACGGTAACATGAAGCACCCACCCGGCGACAAAG |
| | | GAGAGGCCGGCGCTCACCGTAACGCCAAGGAGACCATGACCATCGA |
| | | GAACCCCAAGCTGGAGGATACCGCCGGCGATACGGGTCACAGCAGC |
| | | CTGGAGGCACCGCGGTCTCCCGACACCCTGGCACCCGTGGCCAGCG |
| | | AACGGCTGCCACCCCAACAGAGCGGCGGCCCTGAGGTTGAGACCAA |
| | | GCGGAAGGACAGCATCCTGCCCGAACGGATCGGTGAGCGGCCCAAG |
| | | ATCACCTTACTGCAGAGTAGCAAGGACCGGCTGAGACGGCGGCTGA |
| | | AGGAGAAGGACGAGGTGGCCGTGGAGACAACCACTCCCCAGCAGA |
| | | ACAAGATGGACAAGCTGATCGAGATCCTGAACAGCATGCGGAACAA |
| | | CAGCAGCGACGTGGACACCAAGCTGACCACCTTCATGGAGGAGGCC |
| | | CAGAACAGCACCAACAGCGAGGAGATGCTGGGCGAGATCGTGCGGA |
| | | CCATCTACCAGAAGGCCGTGAGCGACCGGAGCTTCGCCTTCACCGCC |
| | | GCCAAGCTGTGCGACAAGATGGCCCTGTTCATGGTGGAGGGCACCA |
| | | AGTTCCGGAGCTTACTGCTGAATATGCTGCAGAAGGACTTCACCGTG |
| | | CGGGAGGAGCTGCAGCAGCAGGACGTGGAGCGGTGGCTGGGCTTCA |
| | | TCACCTTCCTGTGCGAGGTGTTCGGCACCATGCGGAGCAGCACCGGC |
| | | GAACCCTTCCGGGTGCTGGTGTGCCCCATCTACACCTGCCTGCGGGA |
| | | GTTGCTGCAGAGCCAGGACGTGAAGGAGGACGCCGTGCTGTGCTGC |
| | | AGCATGGAACTGCAGAGCACTGGCCGGCTGCTGGAGGAGCAGCTGC |
| | | CCGAGATGATGACCGAGCTGCTCGCTAGCGCCCGGGACAAGATGCT |
| | | GTGCCCCAGCGAGAGCATGCTGACCCGGAGCCTGCTTCTGGAGGTG |
| | | ATCGAGCTGCACGCCAACAGCTGGAATCCCCTGACCCCTCCCATCAC |
| | | CCAGTACTACAACCGGACCATCCAGAAGCTGACCGCC |
| 32 | MBP--CTIF | MASNFTQFVLVDNGGTGDVTVAPSNFANGIAEWISSNSRSQAYKV |
| | (379-579) (aa) | TCSVRQSSAQNRKYTIKVEVPKGAWRSYLNMELTIPIFATNSDCELIVK |
| | | AMQGLLKDGNPIPSAIAANSGIYGGGGSLNSMRNNSSDVDTKLTTFMEE |
| | | AQNSTNSEEMLGEIVRTIYQKAVSDRSFAFTAAKLCDKMALFMVEGTK |
| | | FRSLLLNMLQKDFTVREELQQQDVERWLGFITFLCEVFGTMRSSTGEPF |
| | | RVLVCPIYTCLRELLQSQDVKEDAVLCCSMELQSTGRLLEEQLPEMMTE |
| | | LLASARDKMLCPSESMLTRSLLLEVIELHANSWN |
| 33 | MBP--CTIF | ATGGCCAGCAACTTCACCCAGTTCGTGCTGGTGGACAACGGCGG |
| | (379-579) (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCCT |
| | | GAACAGCATGAGAAACAACAGCAGCGACGTGGACACCAAGCTGACC |
| | | ACCTTCATGGAGGAGGCCCAGAACAGCACCAACAGCGAGGAGATGC |
| | | TGGGCGAGATCGTGAGAACCATCTACCAGAAGGCCGTGAGCGACAG |
| | | AAGCTTCGCCTTCACCGCCGCCAAGCTGTGCGACAAGATGGCCCTGT |
| | | TCATGGTGGAGGGCACCAAGTTCAGAAGCCTGCTGCTGAACATGCT |
| | | GCAGAAGGACTTCACCGTGAGAGAGGAGCTGCAGCAGCAGGACGTG |
| | | GAGCGATGGCTGGGCTTCATCACCTTCCTGTGCGAGGTGTTCGGCAC |
| | | CATGAGAAGCAGCACCGGCGAGCCTTTCAGAGTGCTGGTGTGCCCT |
| | | ATCTACACCTGCCTGAGAGAGCTGCTGCAGAGCCAGGACGTGAAGG |
| | | AGGACGCCGTGCTGTGCTGCAGCATGGAGCTGCAGAGCACCGGCAG |
| | | ACTGCTGGAGGAGCAGCTGCCTGAGATGATGACCGAGCTGCTGGCC |
| | | AGCGCCAGAGACAAGATGCTGTGCCCTAGCGAGAGCATGCTGACTA |
| | | GATCCCTACTGTTGGAGGTGATCGAGCTGCACGCCAACAGCTGGAA |
| | | C |
| 34 | MBP-_CTIF | MASNFTQFVLVDNGGTGDVTVAPSNFANGIAEWISSNSRSQAYKV |
| | (365-598) (aa) | TCSVRQSSAQNRKYTIKVEVPKGAWRSYLNMELTIPIFATNSDCELIVK |
| | | AMQGLLKDGNPIPSAIAANSGIYGGGGSTTPQQNKMDKLIEILNSMRNN |
| | | SSDVDTKLTTFMEEAQNSTNSEEMLGEIVRTIYQKAVSDRSFAFTAAKL |
| | | CDKMALFMVEGTKFRSLLLNMLQKDFTVREELQQQDVERWLGFITFLC |
| | | EVFGTMRSSTGEPFRVLVCPIYTCLRELLQSQDVKEDAVLCCSMELQST |
| | | GRLLEEQLPEMMTELLASARDKMLCPSESMLTRSLLLEVIELHANSWNP |
| | | LTPPITQYYNRTIQKLTA |
| 35 | MBP-_CTIF | ATGGCCAGCAACTTCACCCAGTTCGTGCTGGTGGACAACGGCGG |
| | (365-598) (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCAC |
| | | CACCCCTCAGCAGAACAAGATGGACAAGCTGATCGAGATCCTGAAC |
| | | AGCATGAGAAACAACAGCAGCGACGTGGACACCAAGCTGACCACCT |
| | | TCATGGAGGAGGCCCAGAACAGCACCAACAGCGAGGAGATGCTGGG |
| | | CGAGATCGTGAGAACCATCTACCAGAAGGCCGTGAGCGACAGAAGC |
| | | TTCGCCTTCACCGCCGCCAAGCTGTGCGACAAGATGGCCCTGTTCAT |
| | | GGTGGAGGGCACCAAGTTCAGAAGCCTGCTGCTGAACATGCTGCAG |
| | | AAGGACTTCACCGTGAGAGAGGAGCTGCAGCAGCAGGACGTGGAGA |
| | | GGTGGCTGGGCTTCATCACCTTCCTGTGCGAGGTGTTCGGCACCATG |
| | | AGAAGCAGCACCGGCGAGCCTTTCAGAGTGCTGGTGTGCCCTATCTA |
| | | CACCTGCCTGAGAGAGCTGCTGCAGAGCCAGGACGTGAAGGAGGAC |
| | | GCCGTGCTGTGCTGCAGCATGGAGCTGCAGAGCACCGGCAGACTGC |
| | | TGGAGGAGCAGCTGCCTGAGATGATGACCGAGCTGCTGGCCAGCGC |
| | | CAGAGACAAGATGCTGTGCCCTAGCGAGAGCATGCTGACGCGCAGC |
| | | CTCCTGCTGGAGGTGATCGAGCTGCACGCCAACAGCTGGAACCCTCT |
| | | GACCCCTCCTATCACCCAGTACTACAACAGAACCATCCAGAAGCTGA |
| | | CCGCC |
| 36 | MBP-eIF4G-mid4 | MASNFTQFVLVDNGGTGDVTVAPSNFANGIAEWISSNSRSQAYKV |
| | (752-993) (aa) | TCSVRQSSAQNRKYTIKVEVPKGAWRSYLNMELTIPIFATNSDCELIVK |
| | | AMQGLLKDGNPIPSAIAANSGIYGGGGSADGSKTQDLFRRVRSILNKLT |
| | | PQMFQQLMKQVTQLAIDTEERLKGVIDLIFEKAISEPNFSVAYANMCRC |
| | | LMALKVPTTEKPTVTVNFRKLLLNRCQKEFEKDKDDDEVFEKKQKEM |
| | | DEAATAEERGRLKEELEEARDIARRRSLGNIKFIGELFKLKMLTEAIMHD |
| | | CVVKLLKNHDEESLECLCRLLTTIGKDLDFEKAKPRMDQYFNQMEKIIK |
| | | EKKTSSRIRFMLQDVLDLRGSNWVPR |
| 37 | MBP-eIF4G-mid4 | ATGGCCAGCAACTTCACCCAGTTCGTGCTGGTGGACAACGGCGG |
| | (752-993) (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCGC |
| | | CGACGGCAGCAAGACCCAGGACCTGTTCAGAAGAGTGAGAAGCATC |
| | | CTGAACAAGCTGACCCCTCAGATGTTCCAGCAGCTGATGAAGCAGG |
| | | TGACCCAGCTGGCCATCGACACCGAGGAGAGACTGAAGGGCGTGAT |
| | | CGACCTGATCTTCGAGAAGGCCATCAGCGAGCCTAACTTCAGCGTGG |
| | | CCTACGCCAACATGTGCCGGTGCCTGATGGCCCTGAAGGTGCCTACC |
| | | ACCGAGAAGCCTACCGTGACCGTGAACTTCAGAAAGCTGCTGCTGA |
| | | ACCGGTGCCAGAAGGAGTTCGAGAAGGACAAGGACGACGACGAGG |
| | | TGTTCGAGAAGAAGCAGAAGGAGATGGACGAGGCCGCCACCGCCGA |
| | | GGAGAGAGGCAGACTGAAGGAGGAGCTGGAGGAGGCCAGAGACAT |
| | | CGCCAGAAGAAGAAGCCTGGGCAACATCAAGTTCATCGGCGAGCTG |
| | | TTCAAGCTGAAGATGCTGACCGAGGCCATCATGCACGACTGCGTGGT |
| | | GAAGCTGCTGAAGAACCACGACGAGGAGAGCCTGGAGTGCCTGTGC |
| | | AGACTGCTGACCACCATCGGCAAGGACCTGGATTTCGAGAAGGCGA |
| | | AGCCTAGAATGGACCAGTACTTCAACCAGATGGAGAAGATCATCAA |
| | | GGAGAAGAAGACCAGCAGCAGAATCAGATTCATGCTGCAGGACGTG |
| | | CTGGACCTGAGAGGCAGCAACTGGGTGCCTAGA |
| 38 | MBP-LacZ | MASNFTQFVLVDNGGTGDVTVAPSNFANGIAEWISSNSRSQAYKV |
| | (control) (aa) | TCSVRQSSAQNRKYTIKVEVPKGAWRSYLNMELTIPIFATNSDCELIVK |
| | | AMQGLLKDGNPIPSAIAANSGIYGGGGSSFTLTNKNVIFVAGLGGIGLDT |
| | | SKELLKRDPVVLQRRDWENPGVTQLNRLAAHPPFASWRNSEEARTDRP |
| | | SQQLRSLNGEWRFAWFPAPEAVPESWLECDLPEADTVVVPSNWQMHG |
| | | YDAPIYTNVTYPITVNPPFVPTENPTGCYSLTFNVDESWLQEGQTRIIFD |
| | | GVNSAFHLWCNGRWVGYGQDSRLPSEFDLSAFLRAGENRLAVMVLR |
| | | WSDGSYLEDQDMWRMSGIFRDVSLLHKPTTQISDFHVATRFNDDFSRA |
| | | VLEAEVQMCGELRDYLRVTVSLWQGETQVASGTAPFGGEIIDERGGYA |
| | | DRVTLRLNVENPKLWSAEIPNLYRAVVELHTADGTLIEAEACDVGFRE |
| | | VRIENGLLLLNGKPLLIRGVNRHEHHPLHGQVMDEQTMVQDILLMKQN |
| | | NFNAVRCSHYPNHPLWYTLCDRYGLYVVDEANIETHGMVPMNRLTDD |
| | | PRWLPAMSERVTRMVQRDRNHPSVIIWSLGNESGHGANHDALYRWIKS |
| | | VDPSRPVQYEGGGADTTATDIICPMYARVDEDQPFPAVPKWSIKKWLS |
| | | LPGETRPLILCEYAHAMGNSLGGFAKYWQAFRQYPRLQGGFVWDWVD |
| | | QSLIKYDENGNPWSAYGGDFGDTPNDRQFCMNGLVFADRTPHPALTEA |
| | | KHQQQFFQFRLSGQTIEVTSEYLFRHSDNELLHWMVALDGKPLASGEV |
| | | PLDVAPQGKQLIELPELPQPESAGQLWLTVRVVQPNATAWSEAGHISA |
| | | WQQWRLAENLSVTLPAASHAIPHLTTSEMDFCIELGNKRWQFNRQSGF |
| | | LSQMWIGDKKQLLTPLRDQFTRAPLDNDIGVSEATRIDPNAWVERWKA |
| | | AGHYQAEAALLQCTADTLADAVLITTAHAWQHQGKTLFISRKTYRIDG |
| | | SGQMAITVDVEVASDTPHPARIGLNCQLAQVAERVNWLGLGPQENYPD |
| | | RLTAACFDRWDLPLSDMYTPYVFPSENGLRCGTRELNYGPHQWRGDF |
| | | QFNISRYSQQQLMETSHRHLLHAEEGTWLNIDGFHMGIGGDDSWSPSV |
| | | SAELQLSAGRYHYQLVWCQK |
| 39 | MBP-LacZ | ATGGCCAGCAACTTCACCCAGTTCGTGCTGGTGGACAACGGCGG |
| | (control) (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCTC |
| | | GTTCACGCTAACGAACAAGAACGTCATCTTCGTAGCGGGACTTGGCG |
| | | GTATCGGCCTAGACACGTCGAAGGAACTACTAAAGCGTGACCCGGT |
| | | AGTCCTCCAACGTCGCGATTGGGAGAACCCGGGCGTAACGCAACTA |
| | | AACCGTCTTGCGGCGCACCCGCCGTTTGCGTCGTGGCGTAACTCGGA |
| | | GGAGGCGCGAACGGATCGTCCGTCGCAACAACTACGTTCGCTCAAC |
| | | GGGGAGTGGCGCTTCGCGTGGTTCCCGGCGCCGGAGGCGGTACCGG |
| | | AGTCGTGGCTCGAGTGCGATCTACCGGAGGCGGACACGGTCGTCGT |
| | | ACCGTCGAACTGGCAAATGCACGGTTACGACGCGCCGATATACACG |
| | | AACGTCACGTACCCGATAACGGTAAACCCGCCGTTCGTCCCGACGG |
| | | AGAACCCGACGGGGTGCTACTCGCTAACGTTCAACGTTGACGAGTC |
| | | GTGGTTGCAAGAGGGTCAAACGCGTATCATATTCGACGGTGTAAACT |
| | | CGGCGTTCCACCTGTGGTGCAACGGGCGCTGGGTAGGGTACGGCCA |
| | | AGACTCGCGTCTACCGTCGGAGTTCGACCTATCGGCGTTCCTACGAG |
| | | CGGGTGAGAACCGGCTAGCGGTCATGGTCCTACGTTGGTCGGACGG |
| | | TTCGTACCTCGAGGACCAAGACATGTGGCGAATGTCGGGTATCTTCC |
| | | GCGACGTATCGCTCCTACACAAGCCGACGACGCAAATCTCGGACTTC |
| | | CACGTCGCGACGCGTTTCAACGACGATTTCTCGCGGGCAGTCCTAGA |
| | | GGCGGAGGTCCAAATGTGCGGGGAGCTACGTGACTACCTCCGTGTC |
| | | ACGGTATCGCTCTGGCAAGGTGAGACGCAAGTAGCGTCGGGTACGG |
| | | CGCCGTTCGGCGGTGAGATCATCGACGAGCGTGGTGGGTACGCGGA |
| | | CCGTGTAACGCTACGTCTAAACGTCGAGAACCCGAAGCTCTGGTCGG |
| | | CGGAGATCCCGAACCTATACCGTGCGGTCGTCGAGCTACATACGGC |
| | | GGACGGGACGCTAATAGAGGCGGAAGCGTGCGACGTCGGGTTTCGA |
| | | GAGGTTCGTATAGAGAACGGGCTGCTACTTCTAAACGGGAAGCCGT |
| | | TGCTCATACGTGGTGTCAACCGTCACGAGCACCACCCGCTACACGGT |
| | | CAAGTAATGGACGAGCAAACGATGGTACAAGACATCCTACTAATGA |
| | | AGCAGAACAACTTCAACGCGGTACGCTGTTCGCATTACCCGAACCAT |
| | | CCGTTGTGGTACACGCTTTGCGACCGATACGGTCTATACGTCGTAGA |
| | | CGAGGCGAACATAGAGACGCACGGGATGGTACCGATGAATCGCCTA |
| | | ACGGACGACCCGCGTTGGCTACCGGCGATGTCGGAGCGAGTCACGC |
| | | GTATGGTCCAACGGGACCGTAACCACCCGTCGGTAATAATCTGGTCG |
| | | CTAGGCAACGAATCGGGGCACGGGGCGAACCACGACGCGCTATACC |
| | | GTTGGATCAAGTCGGTAGACCCGTCGCGTCCGGTACAATACGAAGG |
| | | TGGCGGTGCGGACACGACGGCGACGGACATCATCTGCCCGATGTAC |
| | | GCGCGCGTCGACGAAGACCAACCGTTCCCGGCGGTACCGAAGTGGT |
| | | CGATCAAGAAGTGGCTCTCGTTGCCGGGTGAAACGCGTCCGTTGATA |
| | | CTTTGCGAGTACGCGCACGCGATGGGCAACTCGTTGGGTGGGTTCGC |
| | | GAAGTACTGGCAGGCGTTCCGTCAATACCCGCGTCTACAGGGTGGGT |
| | | TCGTCTGGGACTGGGTAGACCAATCGCTAATCAAGTACGACGAGAA |
| | | CGGCAACCCGTGGTCGGCGTACGGTGGGGACTTCGGGGACACGCCG |
| | | AACGACCGCCAATTCTGTATGAACGGCCTAGTCTTCGCGGACCGAAC |
| | | GCCGCACCCGGCGTTGACGGAGGCGAAGCATCAACAACAATTCTTC |
| | | CAATTCCGTCTATCGGGGCAAACGATCGAGGTAACGTCGGAGTACTT |
| | | GTTCCGGCACTCGGACAACGAGCTACTACACTGGATGGTAGCACTA |
| | | GACGGCAAGCCGCTAGCGTCGGGAGAAGTCCCTTTGGACGTCGCGC |
| | | CGCAAGGTAAGCAACTAATCGAGCTACCGGAGCTACCGCAACCGGA |
| | | GTCGGCGGGTCAACTGTGGTTGACGGTCCGTGTCGTTCAACCGAACG |
| | | CGACGGCGTGGTCGGAGGCGGGTCACATCTCGGCGTGGCAGCAGTG |
| | | GCGTCTAGCGGAGAACCTCTCGGTCACGCTACCGGCGGCGTCGCAC |
| | | GCGATACCGCATCTAACGACGTCGGAGATGGACTTCTGCATCGAGTT |
| | | GGGGAACAAGAGGTGGCAGTTCAACCGTCAATCGGGATTCCTATCG |
| | | CAAATGTGGATAGGTGACAAGAAGCAACTACTAACGCCGCTACGTG |
| | | ATCAGTTCACGCGTGCTCCGCTAGACAACGACATAGGTGTTTCGGAG |
| | | GCGACGCGTATAGACCCGAACGCGTGGGTGGAGCGGTGGAAGGCGG |
| | | CGGGGCACTACCAAGCGGAGGCGGCGCTACTACAGTGCACGGCGGA |
| | | CACGCTAGCGGACGCGGTATTGATCACGACGGCGCACGCGTGGCAA |
| | | CACCAGGGGAAGACGCTATTCATCTCGCGTAAGACGTACCGTATCG |
| | | ACGGTTCGGGCCAAATGGCGATCACGGTCGACGTAGAGGTAGCGTC |
| | | GGACACGCCGCATCCGGCGCGCATCGGTCTAAACTGCCAACTAGCG |
| | | CAAGTAGCGGAGCGTGTAAACTGGCTAGGGCTAGGGCCGCAAGAGA |
| | | ACTATCCGGACCGCCTAACGGCGGCGTGCTTCGACCGTTGGGACCTA |
| | | CCGCTTTCGGACATGTATACCCCGTACGTCTTCCCGTCGGAGAACGG |
| | | GTTGAGGTGCGGGACGCGCGAGCTAAACTACGGGCCGCACCAGTGG |
| | | CGAGGGGACTTCCAATTCAACATATCGCGTTACTCGCAACAACAACT |
| | | AATGGAGACGTCGCACCGTCACCTACTACACGCGGAGGAGGGGACG |
| | | TGGCTAAACATCGACGGGTTCCACATGGGCATAGGTGGGGACGACT |
| | | CGTGGTCGCCGTCGGTCTCGGCGGAGCTCCAACTCTCGGCGGGTCGT |
| | | TACCATTACCAACTAGTTTGGTGCCAGAAG |
| 40 | eIF4G-mid2 (aa) | DPTRLQGINCGPDFTPSFANLGRTTLSTRGPPRGGPGGELPRGPAGL |
| | | GPRRSQQGPRKEPRKIIATVLMTEDIKLNKAEKAWKPSSKRTAADKDR |
| | | GEEDADGSKTQDLFRRVRSILNKLTPQMFQQLMKQVTQLAIDTEERLK |
| | | GVIDLIFEKAISEPNFSVAYANMCRCLMALKVPTTEKPTVTVNFRKLLL |
| | | NRCQKEFEKDKDDDEVFEKKQKEMDEAATAEERGRLKEELEEARDIAR |
| | | RRSLGNIKFIGELFKLKMLTEAIMHDCVVKLLKNHDEESLECLCRLLTTI |
| | | GKDLDFEKAKPRMDQYFNQMEKIIKEKKTSSRIRFMLQDVLDLRGSNW |
| | | VPRRGDQGPKTIDQIHKEAEMEEHREHIKVQQLMAKGSDKRRGGPPGP |
| | | PISRGLPLVDDGGWNTVPISKGSRPIDTSRLTKITKPGSIDSNNQLFAPGG |
| | | RLSWGKGSSGGSGAKPSDAASEAARPATSTLNRFSALQQAV |
| 41 | eIF4G-mid2 (nt) | GACCCTACCAGACTGCAGGGCATCAACTGCGGCCCTGACTTCAC |
| | | TCCTTCTTTCGCAAACCTGGGCAGAACCACCCTGAGCACCAGAGGCC |
| | | CTCCTAGAGGTGGTCCCGGCGGAGAACTCCCCAGGGGTCCTGCCGG |
| | | CCTGGGCCCTAGACGCTCCCAGCAAGGTCCTAGAAAGGAGCCAAGG |
| | | AAGATCATCGCCACCGTGCTGATGACCGAGGACATCAAGCTGAACA |
| | | AAGCTGAGAAGGCCTGGAAGCCTAGCAGCAAGAGAACCGCCGCCGA |
| | | CAAGGACAGAGGCGAGGAGGACGCCGACGGATCCAAGACCCAGGA |
| | | CCTGTTCAGAAGAGTGAGAAGCATCCTCAACAAGCTGACCCCTCAG |
| | | ATGTTCCAGCAGCTGATGAAGCAGGTGACGCAGCTCGCCATCGACA |
| | | CCGAGGAGAGACTGAAGGGCGTGATCGACCTGATCTTTGAGAAGGC |
| | | TATCTCAGAGCCTAACTTCAGCGTGGCCTACGCCAACATGTGCCGTT |
| | | GCCTGATGGCATTGAAGGTGCCAACCACCGAGAAGCCTACTGTGAC |
| | | CGTCAATTTCCGTAAACTGCTGCTGAACCGGTGCCAGAAAGAGTTCG |
| | | AGAAGGATAAGGACGACGACGAGGTCTTCGAGAAGAAACAGAAAG |
| | | AAATGGACGAGGCCGCCACCGCAGAGGAAAGGGGCCGATTAAAGG |
| | | AGGAGCTGGAGGAGGCCAGAGACATCGCCAGACGGCGTTCTCTGGG |
| | | CAACATCAAGTTCATAGGTGAGCTGTTCAAGCTAAAGATGCTCACCG |
| | | AGGCCATAATGCACGACTGCGTGGTGAAGCTACTGAAGAACCACGA |
| | | CGAAGAAAGCCTGGAGTGCCTGTGCAGACTGCTGACCACCATCGGC |
| | | AAGGACCTGGACTTCGAGAAGGCAAAGCCTCGAATGGACCAGTACT |
| | | TCAACCAGATGGAGAAGATTATCAAGGAGAAGAAGACCAGCAGCA |
| | | GAATCAGATTCATGCTGCAGGACGTACTGGACCTGCGCGGAAGCAA |
| | | CTGGGTGCCAAGGAGAGGGGACCAAGGACCAAAGACCATCGACCA |
| | | GATCCACAAGGAAGCGGAGATGGAGGAGCACAGAGAGCACATAAA |
| | | GGTGCAGCAGCTTATGGCCAAGGGCAGCGACAAGCGAAGAGGCGGC |
| | | CCGCCCGGACCTCCTATCAGCAGAGGCCTTCCTCTGGTAGACGACGG |
| | | CGGCTGGAACACCGTGCCTATCTCTAAGGGCTCCAGACCTATCGACA |
| | | CTTCCCGTCTTACCAAGATCACCAAGCCAGGATCTATTGACAGCAAC |
| | | AACCAGCTGTTCGCCCCAGGAGGAAGACTTAGCTGGGGCAAGGGAA |
| | | GTTCCGGCGGATCCGGCGCCAAGCCTTCCGACGCCGCCAGCGAGGC |
| | | TGCCAGACCTGCCACCAGCACCTTGAACCGCTTTTCCGCTCTGCAGC |
| | | AAGCTGTG |
| 42 | eIF4G(dN) | IFASMQKPEGLPHISDVVLDKANKTPLRPLDPTRLQGINCGPDFTPSF |
| | 623-1599 (aa) | ANLGRTTLSTRGPPRGGPGGELPRGPAGLGPRRSQQGPRKEPRKIIATVL |
| | | MTEDIKLNKAEKAWKPSSKRTAADKDRGEEDADGSKTQDLFRRVRSIL |
| | | NKLTPQMFQQLMKQVTQLAIDTEERLKGVIDLIFEKAISEPNFSVAYAN |
| | | MCRCLMALKVPTTEKPTVTVNFRKLLLNRCQKEFEKDKDDDEVFEKK |
| | | QKEMDEAATAEERGRLKEELEEARDIARRRSLGNIKFIGELFKLKMLTE |
| | | AIMHDCVVKLLKNHDEESLECLCRLLTTIGKDLDFEKAKPRMDQYFNQ |
| | | MEKIIKEKKTSSRIRFMLQDVLDLRGSNWVPRRGDQGPKTIDQIHKEAE |
| | | MEEHREHIKVQQLMAKGSDKRRGGPPGPPISRGLPLVDDGGWNTVPIS |
| | | KGSRPIDTSRLTKITKPGSIDSNNQLFAPGGRLSWGKGSSGGSGAKPSDA |
| | | ASEAARPATSTLNRFSALQQAVPTESTDNRRVVQRSSLSRERGEKAGDR |
| | | GDRLERSERGGDRGDRLDRARTPATKRSFSKEVEERSRERPSQPEGLRK |
| | | AASLTEDRDRGRDAVKREAALPPVSPLKAALSEEELEKKSKAIIEEYLHL |
| | | NDMKEAVQCVQELASPSLLFIFVRHGVESTLERSAIAREHMGQLLHQLL |
| | | CAGHLSTAQYYQGLYEILELAEDMEIDIPHVWLYLAELVTPILQEGGVP |
| | | MGELFREITKPLRPLGKAASLLLEILGLLCKSMGPKKVGTLWREAGLSW |
| | | KEFLPEGQDIGAFVAEQKVEYTLGEESEAPGQRALPSEELNRQLEKLLK |
| | | EGSSNQRVFDWIEANLSEQQIVSNTLVRALMTAVCYSAIIFETPLRVDVA |
| | | VLKARAKLLQKYLCDEQKELQALYALQALVVTLEQPPNLLRMFFDAL |
| | | YDEDVVKEDAFYSWESSKDPAEQQGKGVALKSVTAFFKWLREAEEES |
| | | DHN |
| 43 | eIF4G(dN) | ATATTCGCGAGCATGCAGAAGCCTGAAGGTCTGCCTCACATCAG |
| | 623-1599 (nt) | CGACGTGGTGCTGGACAAGGCCAACAAGACCCCTCTTAGACCTCTG |
| | | GACCCTACCAGACTGCAGGGCATCAACTGCGGCCCTGACTTCACTCC |
| | | TTCTTTCGCAAACCTGGGCAGAACCACCCTGAGCACCAGAGGCCCTC |
| | | CTAGAGGTGGTCCCGGCGGAGAACTCCCCAGGGGTCCTGCCGGCCT |
| | | GGGCCCTAGACGCTCCCAGCAAGGTCCTAGAAAGGAGCCAAGGAAG |
| | | ATCATCGCCACCGTGCTGATGACCGAGGACATCAAGCTGAACAAAG |
| | | CTGAGAAGGCCTGGAAGCCTAGCAGCAAGAGAACCGCCGCCGACAA |
| | | GGACAGAGGCGAGGAGGACGCCGACGGATCCAAGACCCAGGACCT |
| | | GTTCAGAAGAGTGAGAAGCATCCTCAACAAGCTGACCCCTCAGATG |
| | | TTCCAGCAGCTGATGAAGCAGGTGACGCAGCTCGCCATCGACACCG |
| | | AGGAGAGACTGAAGGGCGTGATCGACCTGATCTTTGAGAAGGCTAT |
| | | CTCAGAGCCTAACTTCAGCGTGGCCTACGCCAACATGTGCCGTTGCC |
| | | TGATGGCATTGAAGGTGCCAACCACCGAGAAGCCTACTGTGACCGT |
| | | CAATTTCCGTAAACTGCTGCTGAACCGGTGCCAGAAAGAGTTCGAG |
| | | AAGGATAAGGACGACGACGAGGTCTTCGAGAAGAAACAGAAAGAA |
| | | ATGGACGAGGCCGCCACCGCAGAGGAAAGGGGCCGATTAAAGGAG |
| | | GAGCTGGAGGAGGCCAGAGACATCGCCAGACGGCGTTCTCTGGGCA |
| | | ACATCAAGTTCATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTGCCGACCGAGAGCACCGACAACCGGCGAGTTGTGCAGAGAA |
| | | GCAGCCTGAGCAGAGAGAGGGGCGAGAAAGCCGGCGATAGAGGTG |
| | | ACCGACTGGAGAGAAGCGAGAGAGGAGGTGATAGAGGCGACCGGC |
| | | TTGACAGAGCCAGAACCCCTGCCACAAAGCGATCGTTCAGCAAGGA |
| | | GGTCGAAGAGAGGTCCAGGGAGCGCCCTAGCCAGCCGGAAGGACTC |
| | | AGAAAGGCAGCCAGTCTAACAGAGGACCGCGACAGGGGAAGAGAC |
| | | GCCGTGAAGAGGGAGGCCGCACTGCCTCCTGTGAGCCCTCTGAAGG |
| | | CGGCACTGAGCGAAGAAGAACTTGAGAAGAAGAGTAAGGCGATCAT |
| | | CGAGGAGTACCTGCACCTGAACGACATGAAGGAGGCCGTGCAGTGC |
| | | GTGCAAGAGCTCGCGAGCCCATCACTGCTGTTCATCTTCGTCCGGCA |
| | | CGGCGTGGAGTCCACACTGGAAAGATCTGCCATTGCTAGGGAGCAT |
| | | ATGGGCCAGTTGTTGCACCAATTGCTTTGCGCCGGCCACCTAAGTAC |
| | | TGCCCAGTACTATCAGGGTTTATACGAGATCCTCGAACTGGCCGAAG |
| | | ACATGGAGATCGACATCCCTCACGTGTGGCTGTACCTCGCAGAACTG |
| | | GTGACCCCTATCCTGCAGGAGGGCGGCGTTCCAATGGGTGAGTTGTT |
| | | CAGAGAAATCACAAAGCCACTGCGCCCACTGGGCAAGGCGGCTAGT |
| | | CTCCTTCTGGAGATTCTCGGCCTGCTCTGTAAGAGCATGGGTCCAAA |
| | | GAAGGTGGGCACCCTGTGGAGGGAAGCTGGACTCTCCTGGAAGGAA |
| | | TTCCTCCCTGAGGGTCAGGACATCGGCGCCTTCGTGGCCGAACAGAA |
| | | GGTTGAGTACACCCTGGGAGAGGAATCGGAAGCGCCGGGTCAGCGG |
| | | GCTCTGCCGAGTGAGGAGCTCAACAGACAACTCGAGAAGCTCCTCA |
| | | AGGAGGGATCCTCAAATCAGAGAGTGTTCGACTGGATCGAGGCCAA |
| | | TCTCAGCGAGCAGCAAATCGTGAGCAACACGTTGGTTCGCGCTCTCA |
| | | TGACAGCCGTGTGCTACTCAGCCATTATCTTCGAGACGCCTCTCCGC |
| | | GTGGACGTGGCAGTGCTCAAGGCCCGCGCTAAGCTGTTACAGAAGT |
| | | ACCTGTGCGACGAGCAGAAGGAACTGCAGGCCCTGTACGCCCTGCA |
| | | AGCTCTGGTCGTCACACTCGAGCAGCCTCCTAACCTGCTGAGAATGT |
| | | TCTTCGACGCATTGTACGACGAGGACGTTGTGAAGGAAGACGCTTTC |
| | | TACAGCTGGGAGTCTAGTAAGGATCCTGCGGAACAACAAGGCAAGG |
| | | GCGTTGCTCTAAAGAGCGTGACCGCCTTCTTCAAGTGGCTGCGAGAG |
| | | GCTGAGGAAGAGTCGGACCACAAC |
| 44 | eIF4G(dN)- | IFASMQKPEGLPHISDVVLDKANKTPLRPLDPTRLQGINCGPDFTPSF |
| | mid_mutant (aa)_ | ANLGRTTLSTRGPPRGGPGGELPRGPAGLGPRRSQQGPRKEPRKIIATVL |
| | 3A | MTEDIKLNKAEKAWKPSSKRTAADKDRGEEDADGSKTQDLFRRVRSIA |
| | | NKATPQMAQQLMKQVTQLAIDTEERLKGVIDLIFEKAISEPNFSVAYAN |
| | | MCRCLMALKVPTTEKPTVTVNFRKLLLNRCQKEFEKDKDDDEVFEKK |
| | | QKEMDEAATAEERGRLKEELEEARDIARRRSLGNIKFIGELFKLKMLTE |
| | | AIMHDCVVKLLKNHDEESLECLCRLLTTIGKDLDFEKAKPRMDQYFNQ |
| | | MEKIIKEKKTSSRIRFMLQDVLDLRGSNWVPRRGDQGPKTIDQIHKEAE |
| | | MEEHREHIKVQQLMAKGSDKRRGGPPGPPISRGLPLVDDGGWNTVPIS |
| | | KGSRPIDTSRLTKITKPGSIDSNNQLFAPGGRLSWGKGSSGGSGAKPSDA |
| | | ASEAARPATSTLNRFSALQQAVPTESTDNRRVVQRSSLSRERGEKAGDR |
| | | GDRLERSERGGDRGDRLDRARTPATKRSFSKEVEERSRERPSQPEGLRK |
| | | AASLTEDRDRGRDAVKREAALPPVSPLKAALSEEELEKKSKAIIEEYLHL |
| | | NDMKEAVQCVQELASPSLLFIFVRHGVESTLERSAIAREHMGQLLHQLL |
| | | CAGHLSTAQYYQGLYEILELAEDMEIDIPHVWLYLAELVTPILQEGGVP |
| | | MGELFREITKPLRPLGKAASLLLEILGLLCKSMGPKKVGTLWREAGLSW |
| | | KEFLPEGQDIGAFVAEQKVEYTLGEESEAPGQRALPSEELNRQLEKLLK |
| | | EGSSNQRVFDWIEANLSEQQIVSNTLVRALMTAVCYSAIIFETPLRVDVA |
| | | VLKARAKLLQKYLCDEQKELQALYALQALVVTLEQPPNLLRMFFDAL |
| | | YDEDVVKEDAFYSWESSKDPAEQQGKGVALKSVTAFFKWLREAEEES |
| | | DHN |
| 45 | eIF4G(dN)- | ATATTCGCGAGCATGCAGAAGCCTGAAGGTCTGCCTCACATCAG |
| | mid_mutant (nt)_ | CGACGTGGTGCTGGACAAGGCCAACAAGACCCCTCTTAGACCTCTG |
| | 3A | GACCCTACCAGACTGCAGGGCATCAACTGCGGCCCTGACTTCACTCC |
| | | TTCTTTCGCAAACCTGGGCAGAACCACCCTGAGCACCAGAGGCCCTC |
| | | CTAGAGGTGGTCCCGGCGGAGAACTCCCCAGGGGTCCTGCCGGCCT |
| | | GGGCCCTAGACGCTCCCAGCAAGGTCCTAGAAAGGAGCCAAGGAAG |
| | | ATCATCGCCACCGTGCTGATGACCGAGGACATCAAGCTGAACAAAG |
| | | CTGAGAAGGCCTGGAAGCCTAGCAGCAAGAGAACCGCCGCCGACAA |
| | | GGACAGAGGCGAGGAGGACGCCGACGGATCCAAGACCCAGGACCT |
| | | GTTCAGAAGAGTGAGAAGCATCGCGAACAAGGCGACCCCTCAGATG |
| | | GCGCAGCAGCTGATGAAGCAGGTGACGCAGCTCGCCATCGACACCG |
| | | AGGAGAGACTGAAGGGCGTGATCGACCTGATCTTTGAGAAGGCTAT |
| | | CTCAGAGCCTAACTTCAGCGTGGCCTACGCCAACATGTGCCGTTGCC |
| | | TGATGGCATTGAAGGTGCCAACCACCGAGAAGCCTACTGTGACCGT |
| | | CAATTTCCGTAAACTGCTGCTGAACCGGTGCCAGAAAGAGTTCGAG |
| | | AAGGATAAGGACGACGACGAGGTCTTCGAGAAGAAACAGAAAGAA |
| | | ATGGACGAGGCCGCCACCGCAGAGGAAAGGGGCCGATTAAAGGAG |
| | | GAGCTGGAGGAGGCCAGAGACATCGCCAGACGGCGTTCTCTGGGCA |
| | | ACATCAAGTTCATAGGTGAGCTGTTCAAGCTAAAGATGCTCACCGAG |
| | | GCCATAATGCACGACTGCGTGGTGAAGCTACTGAAGAACCACGACG |
| | | AAGAAAGCCTGGAGTGCCTGTGCAGACTGCTGACCACCATCGGCAA |
| | | GGACCTGGACTTCGAGAAGGCAAAGCCTCGAATGGACCAGTACTTC |
| | | AACCAGATGGAGAAGATTATCAAGGAGAAGAAGACCAGCAGCAGA |
| | | ATCAGATTCATGCTGCAGGACGTACTGGACCTGCGCGGAAGCAACT |
| | | GGGTGCCAAGGAGAGGGGACCAAGGACCAAAGACCATCGACCAGA |
| | | TCCACAAGGAAGCGGAGATGGAGGAGCACAGAGAGCACATAAAGG |
| | | TGCAGCAGCTTATGGCCAAGGGCAGCGACAAGCGAAGAGGCGGCCC |
| | | GCCCGGACCTCCTATCAGCAGAGGCCTTCCTCTGGTAGACGACGGCG |
| | | GCTGGAACACCGTGCCTATCTCTAAGGGCTCCAGACCTATCGACACT |
| | | TCCCGTCTTACCAAGATCACCAAGCCAGGATCTATTGACAGCAACAA |
| | | CCAGCTGTTCGCCCCAGGAGGAAGACTTAGCTGGGGCAAGGGAAGT |
| | | TCCGGCGGATCCGGCGCCAAGCCTTCCGACGCCGCCAGCGAGGCTG |
| | | CCAGACCTGCCACCAGCACCTTGAACCGCTTTTCCGCTCTGCAGCAA |
| | | GCTGTGCCGACCGAGAGCACCGACAACCGGCGAGTTGTGCAGAGAA |
| | | GCAGCCTGAGCAGAGAGAGGGGCGAGAAAGCCGGCGATAGAGGTG |
| | | ACCGACTGGAGAGAAGCGAGAGAGGAGGTGATAGAGGCGACCGGC |
| | | TTGACAGAGCCAGAACCCCTGCCACAAAGCGATCGTTCAGCAAGGA |
| | | GGTCGAAGAGAGGTCCAGGGAGCGCCCTAGCCAGCCGGAAGGACTC |
| | | AGAAAGGCAGCCAGTCTAACAGAGGACCGCGACAGGGGAAGAGAC |
| | | GCCGTGAAGAGGGAGGCCGCACTGCCTCCTGTGAGCCCTCTGAAGG |
| | | CGGCACTGAGCGAAGAAGAACTTGAGAAGAAGAGTAAGGCGATCAT |
| | | CGAGGAGTACCTGCACCTGAACGACATGAAGGAGGCCGTGCAGTGC |
| | | GTGCAAGAGCTCGCGAGCCCATCACTGCTGTTCATCTTCGTCCGGCA |
| | | CGGCGTGGAGTCCACACTGGAAAGATCTGCCATTGCTAGGGAGCAT |
| | | ATGGGCCAGTTGTTGCACCAATTGCTTTGCGCCGGCCACCTAAGTAC |
| | | TGCCCAGTACTATCAGGGTTTATACGAGATCCTCGAACTGGCCGAAG |
| | | ACATGGAGATCGACATCCCTCACGTGTGGCTGTACCTCGCAGAACTG |
| | | GTGACCCCTATCCTGCAGGAGGGCGGCGTTCCAATGGGTGAGTTGTT |
| | | CAGAGAAATCACAAAGCCACTGCGCCCACTGGGCAAGGCGGCTAGT |
| | | CTCCTTCTGGAGATTCTCGGCCTGCTCTGTAAGAGCATGGGTCCAAA |
| | | GAAGGTGGGCACCCTGTGGAGGGAAGCTGGACTCTCCTGGAAGGAA |
| | | TTCCTCCCTGAGGGTCAGGACATCGGCGCCTTCGTGGCCGAACAGAA |
| | | GGTTGAGTACACCCTGGGAGAGGAATCGGAAGCGCCGGGTCAGCGG |
| | | GCTCTGCCGAGTGAGGAGCTCAACAGACAACTCGAGAAGCTCCTCA |
| | | AGGAGGGATCCTCAAATCAGAGAGTGTTCGACTGGATCGAGGCCAA |
| | | TCTCAGCGAGCAGCAAATCGTGAGCAACACGTTGGTTCGCGCTCTCA |
| | | TGACAGCCGTGTGCTACTCAGCCATTATCTTCGAGACGCCTCTCCGC |
| | | GTGGACGTGGCAGTGCTCAAGGCCCGCGCTAAGCTGTTACAGAAGT |
| | | ACCTGTGCGACGAGCAGAAGGAACTGCAGGCCCTGTACGCCCTGCA |
| | | AGCTCTGGTCGTCACACTCGAGCAGCCTCCTAACCTGCTGAGAATGT |
| | | TCTTCGACGCATTGTACGACGAGGACGTTGTGAAGGAAGACGCTTTC |
| | | TACAGCTGGGAGTCTAGTAAGGATCCTGCGGAACAACAAGGCAAGG |
| | | GCGTTGCTCTAAAGAGCGTGACCGCCTTCTTCAAGTGGCTGCGAGAG |
| | | GCTGAGGAAGAGTCGGACCACAAC |
| 46 | eIF4GminMut (aa) | DPTRLQGINCGPDFTPSFANLGRTTLSTRGPPRGGPGGELPRGPAGL |
| | | GPRRSQQGPRKEPRKIIATVLMTEDIKLNKAEKAWKPSSKRTAADKDR |
| | | GEEDADGSKTQDLFRRVRSIANKATPQMAQQLMKQVTQLAIDTEERLK |
| | | GVIDLIFEKAISEPNFSVAYANMCRCLMALKVPTTEKPTVTVNFRKLLL |
| | | NRCQKEFEKDKDDDEVFEKKQKEMDEAATAEERGRLKEELEEARDIAR |
| | | RRSLGNIKFIGELFKLKMLTEAIMHDCVVKLLKNHDEESLECLCRLLTTI |
| | | GKDLDFEKAKPRMDQYFNQMEKIIKEKKTSSRIRFMLQDVLDLRGSNW |
| | | VPRRGDQGPKTIDQIHKEAEMEEHREHIKVQQLMAKGSDKRRGGPPGP |
| | | PISRGLPLVDDGGWNTVPISKGSRPIDTSRLTKITKPGSIDSNNQLFAPGG |
| | | RLSWGKGSSGGSGAKPSDAASEAARPATSTLNRFSALQQAV |
| 47 | eIF4GminMut (nt) | GACCCTACCAGACTGCAGGGCATCAACTGCGGCCCTGACTTCAC |
| | | TCCTTCTTTCGCAAACCTGGGCAGAACCACCCTGAGCACCAGAGGCC |
| | | CTCCTAGAGGTGGTCCCGGCGGAGAACTCCCCAGGGGTCCTGCCGG |
| | | CCTGGGCCCTAGACGCTCCCAGCAAGGTCCTAGAAAGGAGCCAAGG |
| | | AAGATCATCGCCACCGTGCTGATGACCGAGGACATCAAGCTGAACA |
| | | AAGCTGAGAAGGCCTGGAAGCCTAGCAGCAAGAGAACCGCCGCCGA |
| | | CAAGGACAGAGGCGAGGAGGACGCCGACGGATCCAAGACCCAGGA |
| | | CCTGTTCAGAAGAGTGAGAAGCATCGCCAACAAGGCGACCCCTCAG |
| | | ATGGCGCAGCAGCTGATGAAGCAGGTGACGCAGCTCGCCATCGACA |
| | | CCGAGGAGAGACTGAAGGGCGTGATCGACCTGATCTTTGAGAAGGC |
| | | TATCTCAGAGCCTAACTTCAGCGTGGCCTACGCCAACATGTGCCGTT |
| | | GCCTGATGGCATTGAAGGTGCCAACCACCGAGAAGCCTACTGTGAC |
| | | CGTCAATTTCCGTAAACTGCTGCTGAACCGGTGCCAGAAAGAGTTCG |
| | | AGAAGGATAAGGACGACGACGAGGTCTTCGAGAAGAAACAGAAAG |
| | | AAATGGACGAGGCCGCCACCGCAGAGGAAAGGGGCCGATTAAAGG |
| | | AGGAGCTGGAGGAGGCCAGAGACATCGCCAGACGGCGTTCTCTGGG |
| | | CAACATCAAGTTCATAGGTGAGCTGTTCAAGCTAAAGATGCTCACCG |
| | | AGGCCATAATGCACGACTGCGTGGTGAAGCTACTGAAGAACCACGA |
| | | CGAAGAAAGCCTGGAGTGCCTGTGCAGACTGCTGACCACCATCGGC |
| | | AAGGACCTGGACTTCGAGAAGGCAAAGCCTCGAATGGACCAGTACT |
| | | TCAACCAGATGGAGAAGATTATCAAGGAGAAGAAGACCAGCAGCA |
| | | GAATCAGATTCATGCTGCAGGACGTACTGGACCTGCGCGGAAGCAA |
| | | CTGGGTGCCAAGGAGAGGGGACCAAGGACCAAAGACCATCGACCA |
| | | GATCCACAAGGAAGCGGAGATGGAGGAGCACAGAGAGCACATAAA |
| | | GGTGCAGCAGCTTATGGCCAAGGGCAGCGACAAGCGAAGAGGCGGC |
| | | CCGCCCGGACCTCCTATCAGCAGAGGCCTTCCTCTGGTAGACGACGG |
| | | CGGCTGGAACACCGTGCCTATCTCTAAGGGCTCCAGACCTATCGACA |
| | | CTTCCCGTCTTACCAAGATCACCAAGCCAGGATCTATTGACAGCAAC |
| | | AACCAGCTGTTCGCCCCAGGAGGAAGACTTAGCTGGGGCAAGGGAA |
| | | GTTCCGGCGGATCCGGCGCCAAGCCTTCCGACGCCGCCAGCGAGGC |
| | | TGCCAGACCTGCCACCAGCACCTTGAACCGCTTTTCCGCTCTGCAGC |
| | | AAGCTGTG |
| 48 | PABP (aa) | NPSAPSYPMASLYVGDLHPDVTEAMLYEKFSPAGPILSIRVCRDMIT |
| | | RRSLGYAYVNFQQPADAERALDTMNFDVIKGKPVRIMWSQRDPSLRKS |
| | | GVGNIFIKNLDKSIDNKALYDTFSAFGNILSCKVVCDENGSKGYGFVHF |
| | | ETQEAAERAIEKMNGMLLNDRKVFVGRFKSRKEREAELGARAKEFTNV |
| | | YIKNFGEDMDDERLKDLFGKFGPALSVKVMTDESGKSKGFGFVSFERH |
| | | EDAQKAVDEMNGKELNGKQIYVGRAQKKVERQTELKRKFEQMKQDRI |
| | | TRYQGVNLYVKNLDDGIDDERLRKEFSPFGTITSAKVMMEGGRSKGFG |
| | | FVCFSSPEEATKAVTEMNGRIVATKPLYVALAQRKEERQAHLTNQYMQ |
| | | RMASVRAVPNPVINPYQPAPPSGYFMAAIPQTQNRAAYYPPSQIAQLRP |
| | | SPRWTAQGARPHPFQNMPGAIRPAAPRPPFSTMRPASSQVPRVMSTQRV |
| | | ANTSTQTMGPRPAAAAAAATPAVRTVPQYKYAAGVRNPQQHLNAQPQ |
| | | VTMQQPAVHVQGQEPLTASMLASAPPQEQKQMLGERLFPLIQAMHPTL |
| | | AGKITGMLLEIDNSELLHMLESPESLRSKVDEAVAVLQAHQAKEAAQK |
| | | AVNSATGVPTV |
| 49 | PABP (nt) | AACCCCAGCGCCCCAAGCTACCCCATGGCCAGCCTGTACGTGGG |
| | | CGACCTGCACCCCGACGTTACCGAGGCCATGCTGTACGAGAAATTTT |
| | | CTCCCGCTGGCCCCATCCTGAGCATCCGGGTGTGCCGGGACATGATT |
| | | ACCCGGCGCTCTCTGGGGTACGCCTACGTGAACTTCCAGCAGCCCGC |
| | | AGACGCCGAACGGGCCCTGGACACCATGAACTTCGACGTGATCAAG |
| | | GGCAAGCCCGTGCGGATCATGTGGAGCCAGCGGGACCCAAGCCTGA |
| | | GAAAGAGCGGCGTGGGCAACATCTTCATCAAGAATCTGGACAAGAG |
| | | CATCGACAACAAGGCCCTGTACGACACCTTCAGCGCCTTCGGCAATA |
| | | TCTTAAGCTGCAAGGTGGTGTGCGACGAGAACGGCAGCAAGGGCTA |
| | | CGGCTTCGTGCACTTCGAAACACAAGAAGCCGCAGAGCGGGCCATC |
| | | GAGAAGATGAACGGCATGCTGCTGAACGACCGGAAGGTGTTCGTGG |
| | | GCCGGTTCAAGAGCCGGAAGGAGCGGGAGGCTGAACTCGGTGCCCG |
| | | GGCCAAGGAGTTCACCAACGTGTACATCAAGAACTTCGGCGAGGAC |
| | | ATGGACGACGAGCGGTTGAAGGACCTGTTCGGCAAGTTCGGCCCCG |
| | | CCCTGTCTGTGAAGGTGATGACCGACGAGAGCGGCAAGTCGAAGGG |
| | | TTTCGGCTTTGTGAGCTTCGAGCGGCACGAGGACGCACAGAAGGCC |
| | | GTGGACGAAATGAACGGTAAGGAGCTTAACGGTAAGCAGATCTACG |
| | | TTGGCCGGGCCCAGAAGAAGGTGGAGCGGCAGACCGAGCTGAAGCG |
| | | GAAGTTCGAGCAGATGAAGCAGGACCGGATCACCCGGTACCAGGGC |
| | | GTGAACCTATACGTGAAGAACCTGGACGACGGCATCGACGACGAAC |
| | | GGCTGCGGAAGGAATTCAGCCCCTTCGGCACCATCACCAGCGCCAA |
| | | GGTAATGATGGAGGGTGGCCGGTCGAAGGGATTCGGTTTCGTGTGCT |
| | | TCAGCAGCCCCGAGGAGGCCACCAAGGCCGTTACTGAGATGAACGG |
| | | CCGGATCGTAGCCACAAAGCCACTTTACGTCGCCCTGGCCCAACGCA |
| | | AGGAGGAACGGCAGGCCCACCTGACCAACCAGTACATGCAGCGGAT |
| | | GGCCAGCGTGCGGGCAGTGCCCAACCCCGTGATCAACCCCTACCAA |
| | | CCCGCACCACCAAGCGGCTACTTCATGGCCGCCATCCCTCAGACCCA |
| | | GAACCGGGCCGCCTACTACCCACCCAGCCAGATCGCCCAGTTGCGG |
| | | CCTAGTCCCCGCTGGACGGCTCAAGGAGCACGACCCCACCCCTTCCA |
| | | GAACATGCCCGGAGCCATCAGACCAGCTGCCCCTAGGCCACCCTTCA |
| | | GCACCATGCGGCCCGCCTCAAGCCAGGTGCCCCGGGTGATGAGCAC |
| | | CCAGCGGGTGGCCAACACCAGCACCCAGACCATGGGCCCTAGACCA |
| | | GCAGCTGCTGCGGCTGCTGCCACTCCTGCCGTCCGGACCGTGCCACA |
| | | GTACAAGTACGCCGCCGGCGTTCGGAACCCACAGCAGCACCTGAAC |
| | | GCCCAGCCCCAGGTGACCATGCAGCAGCCTGCCGTGCACGTGCAGG |
| | | GCCAGGAGCCCCTGACCGCCAGCATGTTAGCAAGCGCCCCTCCACA |
| | | GGAGCAGAAGCAGATGCTGGGCGAAAGGCTGTTTCCCCTGATCCAA |
| | | GCGATGCATCCCACCCTGGCCGGCAAGATCACCGGTATGCTGCTGGA |
| | | GATCGATAATAGCGAGCTGCTGCACATGCTGGAGAGCCCCGAGAGT |
| | | CTTAGATCTAAGGTGGACGAGGCCGTGGCCGTGCTCCAGGCTCACCA |
| | | GGCAAAGGAGGCCGCCCAGAAAGCCGTGAACTCCGCCACAGGCGTG |
| | | CCCACCGTG |
| 50 | MBP-PABP (aa) | MASNFTQFVLVDNGGTGDVTVAPSNFANGIAEWISSNSRSQAYKV |
| | | TCSVRQSSAQNRKYTIKVEVPKGAWRSYLNMELTIPIFATNSDCELIVK |
| | | AMQGLLKDGNPIPSAIAANSGIYGGGGSNPSAPSYPMASLYVGDLHPDV |
| | | TEAMLYEKFSPAGPILSIRVCRDMITRRSLGYAYVNFQQPADAERALDT |
| | | MNFDVIKGKPVRIMWSQRDPSLRKSGVGNIFIKNLDKSIDNKALYDTFS |
| | | AFGNILSCKVVCDENGSKGYGFVHFETQEAAERAIEKMNGMLLNDRKV |
| | | FVGRFKSRKEREAELGARAKEFTNVYIKNFGEDMDDERLKDLFGKFGP |
| | | ALSVKVMTDESGKSKGFGFVSFERHEDAQKAVDEMNGKELNGKQIYV |
| | | GRAQKKVERQTELKRKFEQMKQDRITRYQGVNLYVKNLDDGIDDERL |
| | | RKEFSPFGTITSAKVMMEGGRSKGFGFVCFSSPEEATKAVTEMNGRIVA |
| | | TKPLYVALAQRKEERQAHLTNQYMQRMASVRAVPNPVINPYQPAPPSG |
| | | YFMAAIPQTQNRAAYYPPSQIAQLRPSPRWTAQGARPHPFQNMPGAIRP |
| | | AAPRPPFSTMRPASSQVPRVMSTQRVANTSTQTMGPRPAAAAAAATPA |
| | | VRTVPQYKYAAGVRNPQQHLNAQPQVTMQQPAVHVQGQEPLTASML |
| | | ASAPPQEQKQMLGERLFPLIQAMHPTLAGKITGMLLEIDNSELLHMLES |
| | | PESLRSKVDEAVAVLQAHQAKEAAQKAVNSATGVPTV |
| 51 | MBP-PABP (nt) | ATGGCCAGCAACTTCACCCAGTTCGTGCTGGTGGACAACGGCGG |
| | | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCAA |
| | | CCCCAGCGCCCCAAGCTACCCCATGGCCAGCCTGTACGTGGGCGACC |
| | | TGCACCCCGACGTTACCGAGGCCATGCTGTACGAGAAATTTTCTCCC |
| | | GCTGGCCCCATCCTGAGCATCCGGGTGTGCCGGGACATGATTACCCG |
| | | GCGCTCTCTGGGGTACGCCTACGTGAACTTCCAGCAGCCCGCAGACG |
| | | CCGAACGGGCCCTGGACACCATGAACTTCGACGTGATCAAGGGCAA |
| | | GCCCGTGCGGATCATGTGGAGCCAGCGGGACCCAAGCCTGAGAAAG |
| | | AGCGGCGTGGGCAACATCTTCATCAAGAATCTGGACAAGAGCATCG |
| | | ACAACAAGGCCCTGTACGACACCTTCAGCGCCTTCGGCAATATCTTA |
| | | AGCTGCAAGGTGGTGTGCGACGAGAACGGCAGCAAGGGCTACGGCT |
| | | TCGTGCACTTCGAAACACAAGAAGCCGCAGAGCGGGCCATCGAGAA |
| | | GATGAACGGCATGCTGCTGAACGACCGGAAGGTGTTCGTGGGCCGG |
| | | TTCAAGAGCCGGAAGGAGCGGGAGGCTGAACTCGGTGCCCGGGCCA |
| | | AGGAGTTCACCAACGTGTACATCAAGAACTTCGGCGAGGACATGGA |
| | | CGACGAGCGGTTGAAGGACCTGTTCGGCAAGTTCGGCCCCGCCCTGT |
| | | CTGTGAAGGTGATGACCGACGAGAGCGGCAAGTCGAAGGGTTTCGG |
| | | CTTTGTGAGCTTCGAGCGGCACGAGGACGCACAGAAGGCCGTGGAC |
| | | GAAATGAACGGTAAGGAGCTTAACGGTAAGCAGATCTACGTTGGCC |
| | | GGGCCCAGAAGAAGGTGGAGCGGCAGACCGAGCTGAAGCGGAAGT |
| | | TCGAGCAGATGAAGCAGGACCGGATCACCCGGTACCAGGGCGTGAA |
| | | CCTATACGTGAAGAACCTGGACGACGGCATCGACGACGAACGGCTG |
| | | CGGAAGGAATTCAGCCCCTTCGGCACCATCACCAGCGCCAAGGTAA |
| | | TGATGGAGGGTGGCCGGTCGAAGGGATTCGGTTTCGTGTGCTTCAGC |
| | | AGCCCCGAGGAGGCCACCAAGGCCGTTACTGAGATGAACGGCCGGA |
| | | TCGTAGCCACAAAGCCACTTTACGTCGCCCTGGCCCAACGCAAGGA |
| | | GGAACGGCAGGCCCACCTGACCAACCAGTACATGCAGCGGATGGCC |
| | | AGCGTGCGGGCAGTGCCCAACCCCGTGATCAACCCCTACCAACCCG |
| | | CACCACCAAGCGGCTACTTCATGGCCGCCATCCCTCAGACCCAGAAC |
| | | CGGGCCGCCTACTACCCACCCAGCCAGATCGCCCAGTTGCGGCCTAG |
| | | TCCCCGCTGGACGGCTCAAGGAGCACGACCCCACCCCTTCCAGAAC |
| | | ATGCCCGGAGCCATCAGACCAGCTGCCCCTAGGCCACCCTTCAGCAC |
| | | CATGCGGCCCGCCTCAAGCCAGGTGCCCCGGGTGATGAGCACCCAG |
| | | CGGGTGGCCAACACCAGCACCCAGACCATGGGCCCTAGACCAGCAG |
| | | CTGCTGCGGCTGCTGCCACTCCTGCCGTCCGGACCGTGCCACAGTAC |
| | | AAGTACGCCGCCGGCGTTCGGAACCCACAGCAGCACCTGAACGCCC |
| | | AGCCCCAGGTGACCATGCAGCAGCCTGCCGTGCACGTGCAGGGCCA |
| | | GGAGCCCCTGACCGCCAGCATGTTAGCAAGCGCCCCTCCACAGGAG |
| | | CAGAAGCAGATGCTGGGCGAAAGGCTGTTTCCCCTGATCCAAGCGA |
| | | TGCATCCCACCCTGGCCGGCAAGATCACCGGTATGCTGCTGGAGATC |
| | | GATAATAGCGAGCTGCTGCACATGCTGGAGAGCCCCGAGAGTCTTA |
| | | GATCTAAGGTGGACGAGGCCGTGGCCGTGCTCCAGGCTCACCAGGC |
| | | AAAGGAGGCCGCCCAGAAAGCCGTGAACTCCGCCACAGGCGTGCCC |
| | | ACCGTG |
| 52 | TENT4A (aa) | MASNFTQFVLVDNGGTGDVTVAPSNFANGIAEWISSNSRSQAYKV |
| | | TCSVRQSSAQNRKYTIKVEVPKGAWRSYLNMELTIPIFATNSDCELIVK |
| | | AMQGLLKDGNPIPSAIAANSGIYGGGGSDPRVAWIQPEQKGPANALWM |
| | | QIWETSQGVGRGGSGFASYFCLNSPALDTAAAAGAAGRGSGGLGPALP |
| | | AASPPPPGPTAPAALPPALLTALGPAAEGARRLHKSPSLSSSSSSSSSNAE |
| | | SGTESPGCSSSSSSSASLGRPGGGRGGAFFNFADGAPSAPGTANGHPGPR |
| | | GPAPAGSPSQHQFHPGRRKRENKASTYGLNYLLSGSRAAALSGGGGPG |
| | | AQAPRPGTPWKSRAYSPGIQGLHEEIIDFYNFMSPCPEEAAMRREVVKRI |
| | | ETVVKDLWPTADVQIFGSFSTGLYLPTSDIDLVVFGKWERPPLQLLEQA |
| | | LRKHNVAEPCSIKVLDKATVPIIKLTDQETEVKVDISFNMETGVRAAEFI |
| | | KNYMKKYSLLPYLILVLKQFLLQRDLNEVFTGGISSYSLILMAISFLQLH |
| | | PRIDARRADENLGMLLVEFFELYGRNFNYLKTGIRIKEGGAYIAKEEIM |
| | | KAMTSGYRPSMLCIEDPLLPGNDVGRSSYGAMQVKQVFDYAYIVLSHA |
| | | VSPLARSYPNRDAESTLGRIIKVTQEVIDYRRWIKEKWGSKAHPSPGMD |
| | | SRIKIKERIATCNGEQTQNREPESPYGQRLTLSLSSPQLLSSGSSASSVSSL |
| | | SGSDVDSDTPPCTTPSVYQFSLQAPAPLMAGLPTALPMPSGKPQPTTSRT |
| | | LIMTTNNQTRFTIPPPTLGVAPVPCRQAGVEGTASLKAVHHMSSPAIPSA |
| | | SPNPLSSPHLYHKQHNGMKLSMKGSHGHTQGGGYSSVGSGGVRPPVG |
| | | NRGHHQYNRTGWRRKKHTHTRDSLPVSLSR |
| 53 | TENT4A (nt) | GATCCCCGGGTGGCCTGGATCCAGCCCGAGCAGAAGGGCCCCGC |
| | | CAACGCACTGTGGATGCAGATCTGGGAGACCTCTCAGGGCGTAGGG |
| | | AGGGGAGGAAGCGGCTTCGCCAGCTACTTCTGCCTGAACAGCCCCG |
| | | CTCTGGATACGGCAGCCGCAGCAGGAGCCGCTGGGAGAGGCTCTGG |
| | | AGGCCTGGGACCAGCACTGCCCGCTGCTTCACCACCACCTCCAGGCC |
| | | CTACCGCACCCGCAGCCCTTCCACCCGCCCTGCTGACCGCTCTGGGC |
| | | CCAGCTGCAGAGGGTGCTCGGCGGCTGCACAAGAGCCCCAGCCTGA |
| | | GCAGCAGCAGTAGCAGCTCCAGCAGCAACGCCGAGAGCGGCACAGA |
| | | GAGCCCCGGCTGCAGCAGCAGCAGCTCCAGTTCCGCAAGCCTCGGC |
| | | AGACCAGGCGGTGGCAGAGGCGGTGCATTCTTCAACTTCGCTGACG |
| | | GAGCGCCAAGCGCTCCAGGGACAGCCAACGGACACCCAGGCCCAAG |
| | | AGGTCCGGCACCCGCTGGAAGCCCCAGCCAGCACCAGTTCCACCCC |
| | | GGCAGACGGAAGCGGGAGAACAAGGCCAGCACCTACGGCCTGAACT |
| | | ACCTGCTGAGCGGCTCTCGAGCCGCTGCCCTCTCTGGTGGCGGTGGC |
| | | CCTGGTGCTCAGGCCCCAAGACCTGGCACCCCGTGGAAGTCACGGG |
| | | CCTACAGCCCCGGTATCCAGGGCCTGCACGAGGAGATCATCGACTTC |
| | | TACAACTTCATGAGCCCCTGTCCCGAGGAAGCCGCCATGCGGAGGG |
| | | AGGTGGTGAAGCGGATCGAGACCGTGGTGAAGGACCTGTGGCCCAC |
| | | CGCAGACGTGCAGATCTTCGGCAGCTTCAGCACCGGCCTGTACCTGC |
| | | CCACCAGCGACATCGACCTGGTGGTGTTCGGCAAGTGGGAACGCCC |
| | | TCCCCTGCAGCTGCTGGAGCAGGCCCTGCGGAAGCACAACGTGGCC |
| | | GAGCCCTGCAGCATCAAGGTGCTGGACAAGGCCACCGTGCCCATCA |
| | | TCAAGCTGACCGACCAGGAGACCGAGGTGAAGGTGGACATCAGCTT |
| | | CAACATGGAGACCGGCGTGCGTGCAGCAGAGTTCATCAAGAACTAC |
| | | ATGAAGAAGTACAGCCTGCTGCCCTACCTGATCCTGGTGCTGAAGCA |
| | | GTTCCTGCTGCAGCGGGACCTGAACGAGGTGTTCACCGGCGGCATCA |
| | | GCAGCTACAGCCTGATCCTCATGGCCATCAGCTTCCTGCAGCTGCAC |
| | | CCGCGGATCGACGCAAGGCGGGCCGACGAGAACCTGGGCATGCTGC |
| | | TGGTGGAGTTCTTCGAGCTGTACGGCCGGAACTTCAACTACCTGAAG |
| | | ACCGGCATCCGGATCAAGGAGGGCGGCGCCTACATCGCCAAGGAGG |
| | | AGATCATGAAGGCCATGACCAGCGGCTACCGGCCCAGCATGCTGTG |
| | | CATCGAGGACCCTCTGCTGCCCGGCAACGACGTGGGCCGGAGCAGC |
| | | TACGGCGCCATGCAGGTGAAGCAGGTGTTCGACTACGCCTACATCGT |
| | | GCTGAGCCACGCCGTGAGCCCACTGGCCCGGAGCTACCCCAACCGG |
| | | GACGCCGAGAGCACCCTGGGCCGGATCATCAAGGTGACCCAGGAGG |
| | | TGATCGACTACCGGCGGTGGATCAAGGAGAAGTGGGGCAGCAAGGC |
| | | CCACCCATCTCCCGGCATGGACAGCCGGATCAAGATCAAGGAGCGG |
| | | ATCGCCACCTGCAACGGCGAGCAGACCCAGAACCGGGAGCCCGAAA |
| | | GCCCCTACGGCCAGCGTCTGACCCTGAGCCTGAGCTCTCCCCAGCTG |
| | | CTGAGCAGCGGCAGCAGCGCCAGCAGCGTGAGCAGCCTGAGCGGCA |
| | | GCGACGTGGACAGCGACACCCCACCCTGCACCACCCCAAGCGTGTA |
| | | CCAGTTCTCACTGCAGGCCCCTGCTCCGCTGATGGCTGGCCTGCCTA |
| | | CCGCCCTGCCCATGCCCAGCGGCAAGCCCCAGCCCACCACCAGCCG |
| | | GACCCTGATCATGACCACCAACAACCAGACCCGGTTCACCATCCCAC |
| | | CACCAACCCTGGGCGTGGCTCCTGTGCCTTGTCGGCAGGCCGGAGTG |
| | | GAGGGCACCGCCAGCCTGAAGGCCGTGCACCACATGAGCAGCCCAG |
| | | CCATCCCCAGCGCCAGCCCCAACCCTCTGAGCAGCCCACACCTGTAC |
| | | CACAAGCAGCACAACGGCATGAAGCTGAGCATGAAGGGCAGCCACG |
| | | GACACACCCAGGGCGGAGGGTACTCAAGCGTTGGGAGCGGAGGGGT |
| | | ACGGCCTCCAGTGGGCAACCGGGGCCACCACCAGTACAACAGGACC |
| | | GGCTGGAGACGGAAGAAGCACACCCACACCCGGGACTCTCTGCCCG |
| | | TGAGCCTGAGCCGG |
| 54 | MBP-TENT4A | DPRVAWIQPEQKGPANALWMQIWETSQGVGRGGSGFASYFCLNSP |
| | (aa) | ALDTAAAAGAAGRGSGGLGPALPAASPPPPGPTAPAALPPALLTALGPA |
| | | AEGARRLHKSPSLSSSSSSSSSNAESGTESPGCSSSSSSSASLGRPGGGRG |
| | | GAFFNFADGAPSAPGTANGHPGPRGPAPAGSPSQHQFHPGRRKRENKA |
| | | STYGLNYLLSGSRAAALSGGGGPGAQAPRPGTPWKSRAYSPGIQGLHE |
| | | EIIDFYNFMSPCPEEAAMRREVVKRIETVVKDLWPTADVQIFGSFSTGLY |
| | | LPTSDIDLVVFGKWERPPLQLLEQALRKHNVAEPCSIKVLDKATVPIIKL |
| | | TDQETEVKVDISFNMETGVRAAEFIKNYMKKYSLLPYLILVLKQFLLQR |
| | | DLNEVFTGGISSYSLILMAISFLQLHPRIDARRADENLGMLLVEFFELYG |
| | | RNFNYLKTGIRIKEGGAYIAKEEIMKAMTSGYRPSMLCIEDPLLPGNDV |
| | | GRSSYGAMQVKQVFDYAYIVLSHAVSPLARSYPNRDAESTLGRIIKVTQ |
| | | EVIDYRRWIKEKWGSKAHPSPGMDSRIKIKERIATCNGEQTQNREPESP |
| | | YGQRLTLSLSSPQLLSSGSSASSVSSLSGSDVDSDTPPCTTPSVYQFSLQA |
| | | PAPLMAGLPTALPMPSGKPQPTTSRTLIMTTNNQTRFTIPPPTLGVAPVP |
| | | CRQAGVEGTASLKAVHHMSSPAIPSASPNPLSSPHLYHKQHNGMKLSM |
| | | KGSHGHTQGGGYSSVGSGGVRPPVGNRGHHQYNRTGWRRKKHTHTR |
| | | DSLPVSLSR |
| 55 | MBP-TENT4A (nt) | ATGGCCAGCAACTTCACCCAGTTCGTGCTGGTGGACAACGGCGG |
| | | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCGA |
| | | TCCCCGGGTGGCCTGGATCCAGCCCGAGCAGAAGGGCCCCGCCAAC |
| | | GCACTGTGGATGCAGATCTGGGAGACCTCTCAGGGCGTAGGGAGGG |
| | | GAGGAAGCGGCTTCGCCAGCTACTTCTGCCTGAACAGCCCCGCTCTG |
| | | GATACGGCAGCCGCAGCAGGAGCCGCTGGGAGAGGCTCTGGAGGCC |
| | | TGGGACCAGCACTGCCCGCTGCTTCACCACCACCTCCAGGCCCTACC |
| | | GCACCCGCAGCCCTTCCACCCGCCCTGCTGACCGCTCTGGGCCCAGC |
| | | TGCAGAGGGTGCTCGGCGGCTGCACAAGAGCCCCAGCCTGAGCAGC |
| | | AGCAGTAGCAGCTCCAGCAGCAACGCCGAGAGCGGCACAGAGAGCC |
| | | CCGGCTGCAGCAGCAGCAGCTCCAGTTCCGCAAGCCTCGGCAGACC |
| | | AGGCGGTGGCAGAGGCGGTGCATTCTTCAACTTCGCTGACGGAGCG |
| | | CCAAGCGCTCCAGGGACAGCCAACGGACACCCAGGCCCAAGAGGTC |
| | | CGGCACCCGCTGGAAGCCCCAGCCAGCACCAGTTCCACCCCGGCAG |
| | | ACGGAAGCGGGAGAACAAGGCCAGCACCTACGGCCTGAACTACCTG |
| | | CTGAGCGGCTCTCGAGCCGCTGCCCTCTCTGGTGGCGGTGGCCCTGG |
| | | TGCTCAGGCCCCAAGACCTGGCACCCCGTGGAAGTCACGGGCCTAC |
| | | AGCCCCGGTATCCAGGGCCTGCACGAGGAGATCATCGACTTCTACA |
| | | ACTTCATGAGCCCCTGTCCCGAGGAAGCCGCCATGCGGAGGGAGGT |
| | | GGTGAAGCGGATCGAGACCGTGGTGAAGGACCTGTGGCCCACCGCA |
| | | GACGTGCAGATCTTCGGCAGCTTCAGCACCGGCCTGTACCTGCCCAC |
| | | CAGCGACATCGACCTGGTGGTGTTCGGCAAGTGGGAACGCCCTCCCC |
| | | TGCAGCTGCTGGAGCAGGCCCTGCGGAAGCACAACGTGGCCGAGCC |
| | | CTGCAGCATCAAGGTGCTGGACAAGGCCACCGTGCCCATCATCAAG |
| | | CTGACCGACCAGGAGACCGAGGTGAAGGTGGACATCAGCTTCAACA |
| | | TGGAGACCGGCGTGCGTGCAGCAGAGTTCATCAAGAACTACATGAA |
| | | GAAGTACAGCCTGCTGCCCTACCTGATCCTGGTGCTGAAGCAGTTCC |
| | | TGCTGCAGCGGGACCTGAACGAGGTGTTCACCGGCGGCATCAGCAG |
| | | CTACAGCCTGATCCTCATGGCCATCAGCTTCCTGCAGCTGCACCCGC |
| | | GGATCGACGCAAGGCGGGCCGACGAGAACCTGGGCATGCTGCTGGT |
| | | GGAGTTCTTCGAGCTGTACGGCCGGAACTTCAACTACCTGAAGACCG |
| | | GCATCCGGATCAAGGAGGGCGGCGCCTACATCGCCAAGGAGGAGAT |
| | | CATGAAGGCCATGACCAGCGGCTACCGGCCCAGCATGCTGTGCATC |
| | | GAGGACCCTCTGCTGCCCGGCAACGACGTGGGCCGGAGCAGCTACG |
| | | GCGCCATGCAGGTGAAGCAGGTGTTCGACTACGCCTACATCGTGCTG |
| | | AGCCACGCCGTGAGCCCACTGGCCCGGAGCTACCCCAACCGGGACG |
| | | CCGAGAGCACCCTGGGCCGGATCATCAAGGTGACCCAGGAGGTGAT |
| | | CGACTACCGGCGGTGGATCAAGGAGAAGTGGGGCAGCAAGGCCCAC |
| | | CCATCTCCCGGCATGGACAGCCGGATCAAGATCAAGGAGCGGATCG |
| | | CCACCTGCAACGGCGAGCAGACCCAGAACCGGGAGCCCGAAAGCCC |
| | | CTACGGCCAGCGTCTGACCCTGAGCCTGAGCTCTCCCCAGCTGCTGA |
| | | GCAGCGGCAGCAGCGCCAGCAGCGTGAGCAGCCTGAGCGGCAGCGA |
| | | CGTGGACAGCGACACCCCACCCTGCACCACCCCAAGCGTGTACCAG |
| | | TTCTCACTGCAGGCCCCTGCTCCGCTGATGGCTGGCCTGCCTACCGC |
| | | CCTGCCCATGCCCAGCGGCAAGCCCCAGCCCACCACCAGCCGGACC |
| | | CTGATCATGACCACCAACAACCAGACCCGGTTCACCATCCCACCACC |
| | | AACCCTGGGCGTGGCTCCTGTGCCTTGTCGGCAGGCCGGAGTGGAG |
| | | GGCACCGCCAGCCTGAAGGCCGTGCACCACATGAGCAGCCCAGCCA |
| | | TCCCCAGCGCCAGCCCCAACCCTCTGAGCAGCCCACACCTGTACCAC |
| | | AAGCAGCACAACGGCATGAAGCTGAGCATGAAGGGCAGCCACGGA |
| | | CACACCCAGGGCGGAGGGTACTCAAGCGTTGGGAGCGGAGGGGTAC |
| | | GGCCTCCAGTGGGCAACCGGGGCCACCACCAGTACAACAGGACCGG |
| | | CTGGAGACGGAAGAAGCACACCCACACCCGGGACTCTCTGCCCGTG |
| | | AGCCTGAGCCGG |
| 56 | TENT4B (aa) | DPRIAWFQPEQLGPSNSLWMQIWETTQGLRNLYFNHHCHSSGGAS |
| | | GGGGSSSSSSTATGGSGSSTGSPGGAASAPAPAPAGMYRSGERLLGSHA |
| | | LPAEQRDFLPLETTNNNNNHHQPGAWARRAGSSASSPPSASSSPHPSAA |
| | | VPAADPADSASGSSNKRKRDNKASTYGLNYSLLQPSGGRAAGGGRAD |
| | | GGGVVYSGTPWKRRNYNQGVVGLHEEISDFYEYMSPRPEEEKMRMEV |
| | | VNRIESVIKELWPSADVQIFGSFKTGLYLPTSDIDLVVFGKWENLPLWTL |
| | | EEALRKHKVADEDSVKVLDKATVPIIKLTDSFTEVKVDISFNVQNGVRA |
| | | ADLIKDFTKKYPVLPYLVLVLKQFLLQRDLNEVFTGGIGSYSLFLMAVS |
| | | FLQLHPREDACIPNTNYGVLLIEFFELYGRHFNYLKTGIRIKDGGSYVAK |
| | | DEVQKNMLDGYRPSMLYIEDPLQPGNDVGRSSYGAMQVKQAFDYAY |
| | | VVLSHAVSPIAKYYPNNETESILGRIIRVTDEVATYRDWISKQWGLKNR |
| | | PEPSCNGPVSSSSATQSSSSDVDSDATPCKTPKQLLCRPSTGNRVGSQDV |
| | | SLESSQAVGKMQSTQTTNTSNSTNKSQHGSARLFRSSSKGFQGTTQTSH |
| | | GSLMTNKQHQGKSNNQYYHGKKRKHKRDAPLSDLCR |
| 57 | TENT4B (nt) | GATCCCCGGATCGCCTGGTTCCAGCCCGAGCAGCTAGGCCCAAG |
| | | CAACAGCCTGTGGATGCAGATCTGGGAGACCACCCAGGGCCTGCGG |
| | | AACCTGTACTTCAACCACCACTGTCACAGCTCTGGCGGCGCAAGTGG |
| | | CGGAGGCGGCAGTAGCAGCAGCTCTTCAACAGCAACCGGAGGCAGC |
| | | GGCTCGTCCACCGGCAGTCCGGGTGGAGCCGCCAGTGCCCCAGCTCC |
| | | TGCCCCAGCAGGCATGTACCGGAGCGGCGAACGGCTGCTGGGCAGC |
| | | CACGCTCTGCCTGCCGAGCAGCGGGACTTCCTGCCCCTGGAGACCAC |
| | | CAATAACAACAACAATCACCACCAACCCGGGGCTTGGGCTCGACGA |
| | | GCCGGCTCCAGTGCAAGCAGTCCACCTAGCGCCAGCAGCTCTCCACA |
| | | CCCCAGCGCCGCTGTTCCAGCAGCCGATCCCGCCGACAGTGCCAGCG |
| | | GCAGCAGCAACAAGCGGAAGCGGGACAACAAGGCCAGCACCTACG |
| | | GCCTGAACTACAGCCTGCTGCAACCTTCTGGAGGCCGTGCAGCTGGC |
| | | GGAGGTAGAGCCGACGGTGGCGGTGTGGTGTACAGCGGCACGCCCT |
| | | GGAAGCGGCGGAACTACAACCAGGGCGTGGTGGGCCTGCACGAGGA |
| | | GATCAGCGACTTCTACGAGTACATGAGCCCACGGCCCGAAGAGGAG |
| | | AAGATGCGGATGGAGGTGGTGAACCGGATCGAGAGCGTGATCAAGG |
| | | AGCTGTGGCCTAGCGCCGACGTGCAGATCTTCGGCAGCTTCAAGACC |
| | | GGCCTGTACCTGCCCACCAGCGACATCGACCTGGTGGTGTTCGGCAA |
| | | GTGGGAGAACCTGCCCCTGTGGACCCTGGAGGAGGCCCTGCGGAAA |
| | | CACAAGGTGGCCGACGAGGACAGCGTGAAGGTGCTGGACAAGGCCA |
| | | CCGTGCCCATCATCAAGCTGACCGACAGCTTCACCGAGGTGAAGGT |
| | | GGACATCAGCTTCAACGTGCAGAACGGCGTGAGGGCAGCCGACCTG |
| | | ATCAAGGACTTCACCAAGAAGTACCCCGTGCTGCCCTACCTGGTGCT |
| | | GGTGCTGAAGCAGTTCCTGCTGCAGCGGGACCTGAACGAGGTGTTC |
| | | ACCGGCGGCATCGGCAGCTACAGCCTGTTCCTGATGGCCGTGAGCTT |
| | | CCTGCAGCTGCATCCCCGTGAGGACGCCTGCATCCCCAACACCAACT |
| | | ACGGCGTGCTGCTGATCGAGTTCTTCGAGCTGTACGGCCGGCACTTC |
| | | AACTACCTGAAGACCGGCATCCGGATCAAGGACGGCGGCAGCTACG |
| | | TGGCCAAGGACGAGGTGCAGAAGAACATGCTGGACGGCTACCGGCC |
| | | CAGCATGCTGTACATCGAGGACCCACTGCAGCCCGGCAACGACGTG |
| | | GGCCGGAGCAGCTACGGCGCCATGCAGGTGAAGCAGGCCTTCGACT |
| | | ACGCCTACGTGGTGCTGAGCCACGCCGTGAGCCCCATCGCCAAGTAC |
| | | TACCCCAACAACGAGACCGAGAGCATCCTGGGCCGGATCATCCGGG |
| | | TGACCGACGAGGTGGCCACCTACCGGGACTGGATCAGCAAGCAGTG |
| | | GGGCCTGAAGAACCGGCCCGAGCCTAGCTGCAACGGCCCCGTGAGC |
| | | TCAAGCAGCGCCACCCAGAGCAGCAGCAGCGACGTGGACAGCGACG |
| | | CCACTCCCTGCAAGACCCCTAAGCAGCTGCTGTGCCGTCCCAGCACC |
| | | GGCAACCGGGTGGGCAGCCAGGACGTGAGCCTGGAGAGCAGCCAG |
| | | GCCGTGGGCAAGATGCAGAGCACCCAGACCACCAACACCAGCAACA |
| | | GCACCAACAAGAGCCAACACGGCAGCGCACGGCTGTTCCGGAGCAG |
| | | CAGCAAGGGCTTCCAGGGCACCACCCAGACCAGCCACGGCAGCCTG |
| | | ATGACCAACAAGCAGCACCAGGGCAAGAGCAACAACCAGTACTACC |
| | | ACGGCAAGAAGCGGAAGCACAAGCGGGACGCTCCCCTGAGCGACCT |
| | | GTGCCGG |
| 58 | MBP-TENT4B | MASNFTQFVLVDNGGTGDVTVAPSNFANGIAEWISSNSRSQAYKV |
| | (aaa) | TCSVRQSSAQNRKYTIKVEVPKGAWRSYLNMELTIPIFATNSDCELIVK |
| | | AMQGLLKDGNPIPSAIAANSGIYGGGGSDPRIAWFQPEQLGPSNSLWMQ |
| | | IWETTQGLRNLYFNHHCHSSGGASGGGGSSSSSSTATGGSGSSTGSPGG |
| | | AASAPAPAPAGMYRSGERLLGSHALPAEQRDFLPLETTNNNNNHHQPG |
| | | AWARRAGSSASSPPSASSSPHPSAAVPAADPADSASGSSNKRKRDNKAS |
| | | TYGLNYSLLQPSGGRAAGGGRADGGGVVYSGTPWKRRNYNQGVVGL |
| | | HEEISDFYEYMSPRPEEEKMRMEVVNRIESVIKELWPSADVQIFGSFKTG |
| | | LYLPTSDIDLVVFGKWENLPLWTLEEALRKHKVADEDSVKVLDKATVP |
| | | IIKLTDSFTEVKVDISFNVQNGVRAADLIKDFTKKYPVLPYLVLVLKQFL |
| | | LQRDLNEVFTGGIGSYSLFLMAVSFLQLHPREDACIPNTNYGVLLIEFFE |
| | | LYGRHFNYLKTGIRIKDGGSYVAKDEVQKNMLDGYRPSMLYIEDPLQP |
| | | GNDVGRSSYGAMQVKQAFDYAYVVLSHAVSPIAKYYPNNETESILGRII |
| | | RVTDEVATYRDWISKQWGLKNRPEPSCNGPVSSSSATQSSSSDVDSDAT |
| | | PCKTPKQLLCRPSTGNRVGSQDVSLESSQAVGKMQSTQTTNTSNSTNKS |
| | | QHGSARLFRSSSKGFQGTTQTSHGSLMTNKQHQGKSNNQYYHGKKRK |
| | | HKRDAPLSDLCR |
| 59 | MBP-TENT4B (nt) | ATGGCCAGCAACTTCACCCAGTTCGTGCTGGTGGACAACGGCGG |
| | | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCGA |
| | | TCCCCGGATCGCCTGGTTCCAGCCCGAGCAGCTAGGCCCAAGCAAC |
| | | AGCCTGTGGATGCAGATCTGGGAGACCACCCAGGGCCTGCGGAACC |
| | | TGTACTTCAACCACCACTGTCACAGCTCTGGCGGCGCAAGTGGCGGA |
| | | GGCGGCAGTAGCAGCAGCTCTTCAACAGCAACCGGAGGCAGCGGCT |
| | | CGTCCACCGGCAGTCCGGGTGGAGCCGCCAGTGCCCCAGCTCCTGCC |
| | | CCAGCAGGCATGTACCGGAGCGGCGAACGGCTGCTGGGCAGCCACG |
| | | CTCTGCCTGCCGAGCAGCGGGACTTCCTGCCCCTGGAGACCACCAAT |
| | | AACAACAACAATCACCACCAACCCGGGGCTTGGGCTCGACGAGCCG |
| | | GCTCCAGTGCAAGCAGTCCACCTAGCGCCAGCAGCTCTCCACACCCC |
| | | AGCGCCGCTGTTCCAGCAGCCGATCCCGCCGACAGTGCCAGCGGCA |
| | | GCAGCAACAAGCGGAAGCGGGACAACAAGGCCAGCACCTACGGCCT |
| | | GAACTACAGCCTGCTGCAACCTTCTGGAGGCCGTGCAGCTGGCGGA |
| | | GGTAGAGCCGACGGTGGCGGTGTGGTGTACAGCGGCACGCCCTGGA |
| | | AGCGGCGGAACTACAACCAGGGCGTGGTGGGCCTGCACGAGGAGAT |
| | | CAGCGACTTCTACGAGTACATGAGCCCACGGCCCGAAGAGGAGAAG |
| | | ATGCGGATGGAGGTGGTGAACCGGATCGAGAGCGTGATCAAGGAGC |
| | | TGTGGCCTAGCGCCGACGTGCAGATCTTCGGCAGCTTCAAGACCGGC |
| | | CTGTACCTGCCCACCAGCGACATCGACCTGGTGGTGTTCGGCAAGTG |
| | | GGAGAACCTGCCCCTGTGGACCCTGGAGGAGGCCCTGCGGAAACAC |
| | | AAGGTGGCCGACGAGGACAGCGTGAAGGTGCTGGACAAGGCCACCG |
| | | TGCCCATCATCAAGCTGACCGACAGCTTCACCGAGGTGAAGGTGGA |
| | | CATCAGCTTCAACGTGCAGAACGGCGTGAGGGCAGCCGACCTGATC |
| | | AAGGACTTCACCAAGAAGTACCCCGTGCTGCCCTACCTGGTGCTGGT |
| | | GCTGAAGCAGTTCCTGCTGCAGCGGGACCTGAACGAGGTGTTCACC |
| | | GGCGGCATCGGCAGCTACAGCCTGTTCCTGATGGCCGTGAGCTTCCT |
| | | GCAGCTGCATCCCCGTGAGGACGCCTGCATCCCCAACACCAACTACG |
| | | GCGTGCTGCTGATCGAGTTCTTCGAGCTGTACGGCCGGCACTTCAAC |
| | | TACCTGAAGACCGGCATCCGGATCAAGGACGGCGGCAGCTACGTGG |
| | | CCAAGGACGAGGTGCAGAAGAACATGCTGGACGGCTACCGGCCCAG |
| | | CATGCTGTACATCGAGGACCCACTGCAGCCCGGCAACGACGTGGGC |
| | | CGGAGCAGCTACGGCGCCATGCAGGTGAAGCAGGCCTTCGACTACG |
| | | CCTACGTGGTGCTGAGCCACGCCGTGAGCCCCATCGCCAAGTACTAC |
| | | CCCAACAACGAGACCGAGAGCATCCTGGGCCGGATCATCCGGGTGA |
| | | CCGACGAGGTGGCCACCTACCGGGACTGGATCAGCAAGCAGTGGGG |
| | | CCTGAAGAACCGGCCCGAGCCTAGCTGCAACGGCCCCGTGAGCTCA |
| | | AGCAGCGCCACCCAGAGCAGCAGCAGCGACGTGGACAGCGACGCCA |
| | | CTCCCTGCAAGACCCCTAAGCAGCTGCTGTGCCGTCCCAGCACCGGC |
| | | AACCGGGTGGGCAGCCAGGACGTGAGCCTGGAGAGCAGCCAGGCCG |
| | | TGGGCAAGATGCAGAGCACCCAGACCACCAACACCAGCAACAGCAC |
| | | CAACAAGAGCCAACACGGCAGCGCACGGCTGTTCCGGAGCAGCAGC |
| | | AAGGGCTTCCAGGGCACCACCCAGACCAGCCACGGCAGCCTGATGA |
| | | CCAACAAGCAGCACCAGGGCAAGAGCAACAACCAGTACTACCACGG |
| | | CAAGAAGCGGAAGCACAAGCGGGACGCTCCCCTGAGCGACCTGTGC |
| | | CGG |
| 60 | eIF4G-fl (aa) | NSQPQTRSPFFQRPQIQPPRATIPNSSPSIRPGAQTPTAVYQANQHIM |
| | | MVNHLPMPYPVPQGPQYCIPQYRHSGPPYVGPPQQYPVQPPGPGPFYPG |
| | | PGPGDFPNAYGTPFYPSQPVYQSAPIIVPTQQQPPPAKREKKTIRIRDPNQ |
| | | GGKDITEEIMSGGGSRNPTPPIGRPTSTPTPPQQLPSQVPEHSPVVYGTVE |
| | | SAHLAASTPVTAASDQKQEEKPKPDPVLKSPSPVLRLVLSGEKKEQEGQ |
| | | TSETTAIVSIAELPLPPSPTTVSSVARSTIAAPTSSALSSQPIFTTAIDDRCE |
| | | LSSPREDTIPIPSLTSCTETSDPLPTNENDDDICKKPCSVAPNDIPLVSSTN |
| | | LINEINGVSEKLSATESIVEIVKQEVLPLTLELEILENPPEEMKLECIPAPIT |
| | | PSTVPSFPPTPPTPPASPPHTPVIVPAAATTVSSPSAAITVQRVLEEDESIRT |
| | | CLSEDAKEIQNKIEVEADGQTEEILDSQNLNSRRSPVPAQIAITVPKTWK |
| | | KPKDRTRTTEEMLEAELELKAEEELSIDKVLESEQDKMSQGFHPERDPS |
| | | DLKKVKAVEENGEEAEPVRNGAESVSEGEGIDANSGSTDSSGDGVTFPF |
| | | KPESWKPTDTEGKKQYDREFLLDFQFMPACIQKPEGLPPISDVVLDKIN |
| | | QPKLPMRTLDPRILPRGPDFTPAFADFGRQTPGGRGVPLLNVGSRRSQP |
| | | GQRREPRKIITVSVKEDVHLKKAENAWKPSQKRDSQADDPENIKTQELF |
| | | RKVRSILNKLTPQMFNQLMKQVSGLTVDTEERLKGVIDLVFEKAIDEPS |
| | | FSVAYANMCRCLVTLKVPMADKPGNTVNFRKLLLNRCQKEFEKDKAD |
| | | DDVFEKKQKELEAASAPEERTRLHDELEEAKDKARRRSIGNIKFIGELFK |
| | | LKMLTEAIMHDCVVKLLKNHDEESLECLCRLLTTIGKDLDFEKAKPRM |
| | | DQYFNQMEKIVKERKTSSRIRFMLQDVIDLRLCNWVSRRADQGPKTIEQ |
| | | IHKEAKIEEQEEQRKVQQLMTKEKRRPGVQRVDEGGWNTVQGAKNSR |
| | | VLDPSKFLKITKPTIDEKIQLVPKAQLGSWGKGSSGGAKASETDALRSSA |
| | | SSLNRFSALQPPAPSGSTPSTPVEFDSRRTLTSRGSMGREKNDKPLPSAT |
| | | ARPNTFMRGGSSKDLLDNQSQEEQRREMLETVKQLTGGVDVERNSTEA |
| | | ERNKTRESAKPEISAMSAHDKAALSEEELERKSKSIIDEFLHINDFKEAM |
| | | QCVEELNAQGLLHVFVRVGVESTLERSQITRDHMGQLLYQLVQSEKLS |
| | | KQDFFKGFSETLELADDMAIDIPHIWLYLAELVTPMLKEGGISMRELTIE |
| | | FSKPLLPVGRAGVLLSEILHLLCKQMSHKKVGALWREADLSWKDFLPE |
| | | GEDVHNFLLEQKLDFIESDSPCSSEALSKKELSAEELYKRLEKLIIEDKAN |
| | | DEQIFDWVEANLDEIQMSSPTFLRALMTAVCKAAIIADSSTFRVDTAVIK |
| | | QRVPILLKYLDSDTEKELQALYALQASIVKLDQPANLLRMFFDCLYDEE |
| | | VISEDAFYKWESSKDPAEQNGKGVALKSVTAFFTWLREAEEESEDN |
| 61 | eIF4G-fl (nt) | AACAGCCAACCCCAGACACGGAGCCCCTTCTTCCAGCGGCCCCA |
| | | GATCCAGCCACCACGGGCAACCATCCCCAACAGCAGCCCCAGCATC |
| | | CGACCCGGCGCACAGACACCCACCGCCGTGTACCAGGCCAACCAGC |
| | | ACATCATGATGGTGAACCACCTGCCCATGCCCTACCCCGTGCCACAA |
| | | GGCCCTCAGTACTGCATCCCACAGTACCGGCACAGCGGTCCTCCCTA |
| | | CGTGGGGCCGCCACAGCAGTACCCCGTTCAGCCTCCTGGACCGGGA |
| | | CCCTTTTATCCGGGACCTGGACCCGGAGACTTCCCCAACGCCTACGG |
| | | CACACCCTTCTACCCCAGCCAGCCAGTGTACCAGAGCGCGCCCATCA |
| | | TCGTGCCCACCCAGCAGCAGCCTCCACCCGCCAAGCGGGAGAAGAA |
| | | GACCATCCGGATCCGGGACCCCAACCAGGGCGGCAAGGACATCACC |
| | | GAGGAGATCATGAGCGGAGGCGGCAGTCGGAACCCCACACCGCCCA |
| | | TCGGCAGACCCACCAGCACCCCAACTCCACCACAGCAGCTGCCCAG |
| | | CCAGGTGCCCGAGCACAGCCCCGTGGTGTACGGCACCGTGGAAAGC |
| | | GCCCACCTGGCCGCTAGCACCCCTGTGACCGCCGCCAGCGACCAGA |
| | | AGCAGGAGGAGAAGCCCAAACCCGACCCCGTGCTGAAGTCTCCCAG |
| | | CCCCGTGCTGCGTCTGGTGCTGAGCGGCGAGAAGAAGGAGCAGGAG |
| | | GGCCAGACCAGCGAGACAACCGCCATCGTGAGCATCGCCGAGCTGC |
| | | CACTGCCTCCCAGTCCCACCACCGTGAGCAGCGTGGCCCGGAGCACC |
| | | ATCGCAGCGCCCACAAGCAGCGCCCTGAGCAGCCAGCCCATCTTCA |
| | | CCACCGCCATCGACGACCGGTGCGAGCTGAGCAGTCCCCGGGAGGA |
| | | CACCATCCCCATCCCCAGCCTGACCAGCTGCACCGAGACCAGCGACC |
| | | CACTGCCCACCAACGAGAACGACGACGACATCTGCAAGAAGCCCTG |
| | | CAGCGTGGCACCCAACGACATCCCGCTGGTGAGCAGCACCAACCTG |
| | | ATCAACGAGATCAACGGCGTTAGCGAGAAACTGTCTGCCACCGAGA |
| | | GCATCGTGGAGATCGTCAAGCAGGAGGTGCTGCCCCTGACCCTGGA |
| | | GCTAGAGATCCTGGAGAACCCTCCAGAGGAGATGAAGCTGGAGTGC |
| | | ATCCCCGCCCCAATCACCCCGAGCACCGTGCCCAGCTTTCCTCCAAC |
| | | CCCACCCACCCCACCAGCCAGTCCACCTCACACTCCCGTGATCGTGC |
| | | CTGCCGCCGCTACCACCGTGTCTAGCCCCAGCGCCGCCATCACCGTC |
| | | CAGCGGGTACTGGAGGAGGACGAGAGCATCCGGACCTGCCTGAGCG |
| | | AAGACGCCAAGGAGATCCAGAACAAGATCGAGGTGGAGGCCGACG |
| | | GCCAGACCGAGGAAATCCTGGACAGCCAGAACCTGAATTCCCGGCG |
| | | AAGCCCTGTGCCCGCACAGATCGCCATCACAGTGCCCAAGACCTGG |
| | | AAGAAGCCCAAGGACCGGACCCGGACCACAGAGGAAATGCTGGAG |
| | | GCCGAGCTGGAGCTGAAGGCCGAGGAAGAGCTGAGCATCGACAAG |
| | | GTGCTGGAGAGCGAGCAGGACAAGATGAGCCAGGGCTTCCACCCTG |
| | | AGCGGGACCCCAGCGACCTGAAGAAGGTGAAGGCCGTGGAGGAGA |
| | | ACGGCGAGGAGGCAGAGCCTGTGCGGAACGGGGCCGAGAGCGTGA |
| | | GCGAGGGCGAGGGCATCGACGCCAACAGCGGCAGCACTGACAGCA |
| | | GCGGCGACGGCGTGACCTTCCCCTTCAAGCCCGAAAGCTGGAAGCC |
| | | CACCGACACCGAAGGAAAGAAGCAGTACGACCGGGAGTTCCTGCTG |
| | | GACTTCCAGTTCATGCCCGCCTGCATCCAGAAACCCGAGGGGCTGCC |
| | | ACCAATCAGCGACGTGGTGCTGGACAAGATCAACCAGCCCAAGCTG |
| | | CCCATGCGGACCCTGGATCCTCGGATCCTTCCCAGAGGCCCCGACTT |
| | | CACTCCCGCCTTCGCCGACTTCGGCAGGCAGACCCCAGGTGGTCGGG |
| | | GTGTGCCCCTGCTGAACGTGGGCAGCCGTCGGAGCCAGCCTGGCCA |
| | | ACGGCGGGAACCCCGGAAGATCATCACCGTGAGCGTGAAGGAGGAC |
| | | GTGCATCTGAAGAAGGCCGAGAACGCCTGGAAACCCAGCCAGAAGC |
| | | GGGACAGCCAAGCCGACGACCCCGAGAACATCAAGACCCAGGAGCT |
| | | GTTCCGGAAGGTTCGGAGCATCCTGAACAAGCTGACACCCCAGATG |
| | | TTCAACCAGCTGATGAAGCAGGTGAGCGGCCTGACCGTGGACACCG |
| | | AGGAGCGGCTGAAGGGCGTGATCGACCTGGTGTTTGAGAAAGCCAT |
| | | TGACGAGCCCAGCTTCAGCGTGGCCTACGCCAACATGTGCCGGTGCC |
| | | TGGTGACCCTGAAGGTGCCCATGGCCGACAAGCCCGGCAACACCGT |
| | | GAACTTCCGGAAGCTGCTGCTGAACCGGTGCCAGAAGGAGTTCGAG |
| | | AAGGACAAGGCAGACGACGACGTGTTCGAGAAGAAGCAGAAAGAG |
| | | TTAGAGGCCGCAAGCGCCCCTGAGGAGCGGACCCGGCTGCACGACG |
| | | AGCTGGAGGAGGCCAAAGACAAGGCCCGGCGGAGGAGCATCGGCA |
| | | ACATCAAGTTCATCGGCGAGCTGTTCAAGCTGAAGATGCTGACCGA |
| | | GGCCATCATGCACGACTGCGTGGTGAAGCTGCTGAAGAACCACGAC |
| | | GAGGAGAGCCTGGAGTGCCTGTGCCGGCTGCTGACCACCATCGGCA |
| | | AGGACCTGGACTTCGAGAAGGCCAAGCCCCGGATGGACCAGTACTT |
| | | CAACCAGATGGAGAAGATCGTGAAGGAGCGGAAGACCAGCAGCCG |
| | | GATCCGGTTCATGCTGCAGGACGTTATCGACCTGCGGCTGTGCAACT |
| | | GGGTGAGCCGGAGGGCTGACCAGGGCCCCAAGACCATCGAGCAGAT |
| | | CCACAAGGAGGCTAAGATCGAGGAGCAGGAAGAGCAGCGGAAGGT |
| | | GCAGCAGCTGATGACCAAAGAGAAGCGGAGGCCTGGCGTGCAGCGA |
| | | GTGGACGAGGGCGGCTGGAACACCGTGCAGGGCGCCAAGAACAGCC |
| | | GGGTGCTGGACCCCAGCAAGTTCCTGAAGATCACCAAGCCCACCAT |
| | | CGACGAGAAGATCCAGCTGGTGCCCAAGGCCCAGCTCGGCAGCTGG |
| | | GGCAAGGGCAGCTCAGGTGGCGCCAAGGCCAGCGAGACCGACGCCC |
| | | TGAGAAGCAGCGCCAGCAGCCTGAACCGGTTCTCTGCCCTGCAGCCT |
| | | CCTGCCCCTAGCGGCTCCACCCCAAGCACACCCGTGGAGTTCGACAG |
| | | CCGGCGGACCCTGACCAGCCGGGGCTCCATGGGTCGCGAGAAGAAC |
| | | GACAAGCCCTTACCCAGCGCAACCGCTCGGCCCAACACCTTCATGCG |
| | | GGGCGGCAGCTCCAAGGACCTGCTGGACAACCAGAGCCAGGAGGAG |
| | | CAGCGGCGGGAGATGCTGGAGACCGTGAAGCAGCTGACCGGAGGCG |
| | | TGGACGTGGAGCGGAACAGCACCGAGGCCGAGCGGAACAAGACCC |
| | | GGGAGAGCGCCAAGCCCGAGATCAGCGCCATGAGCGCCCACGACAA |
| | | GGCCGCCCTGAGCGAGGAGGAGCTGGAACGGAAGAGCAAGAGCAT |
| | | CATCGACGAGTTCCTGCACATCAACGACTTCAAGGAGGCCATGCAGT |
| | | GCGTGGAGGAGCTGAACGCCCAGGGCCTGCTGCACGTGTTCGTGCG |
| | | GGTGGGCGTGGAGAGCACCCTGGAGCGGAGCCAGATCACCCGGGAC |
| | | CACATGGGCCAGCTGCTGTACCAACTGGTGCAGAGCGAGAAGCTGA |
| | | GCAAGCAGGACTTCTTCAAGGGCTTCAGCGAGACTCTGGAGCTGGC |
| | | CGACGACATGGCCATCGACATTCCCCACATCTGGCTGTACCTGGCCG |
| | | AGCTTGTGACCCCAATGCTGAAGGAGGGCGGCATCAGCATGCGGGA |
| | | GCTGACCATCGAGTTCAGCAAGCCCCTCTTGCCAGTCGGCAGAGCAG |
| | | GAGTGCTGCTGAGCGAGATCCTGCACCTGCTGTGCAAGCAGATGAG |
| | | CCACAAGAAGGTGGGCGCCCTGTGGAGGGAGGCCGACCTGAGCTGG |
| | | AAGGACTTCCTGCCCGAGGGCGAAGACGTGCACAACTTCTTGCTGG |
| | | AGCAGAAGCTGGACTTCATCGAGAGCGACAGCCCCTGCAGCAGCGA |
| | | GGCCCTGAGCAAGAAGGAACTGAGCGCCGAGGAGCTGTACAAGCGG |
| | | CTGGAGAAGCTGATCATCGAGGACAAGGCCAACGACGAGCAGATCT |
| | | TCGACTGGGTGGAGGCCAACCTGGACGAGATCCAGATGAGCAGCCC |
| | | CACCTTCCTGCGGGCCCTGATGACCGCCGTGTGCAAGGCCGCCATCA |
| | | TCGCCGACAGCAGCACCTTCCGGGTGGACACCGCCGTGATCAAGCA |
| | | GCGGGTGCCCATCCTGCTGAAGTACCTGGACAGCGACACCGAGAAG |
| | | GAGCTGCAGGCCCTGTACGCCCTGCAGGCCAGCATCGTGAAGCTGG |
| | | ACCAGCCCGCCAACCTGCTGCGGATGTTCTTCGACTGCCTGTACGAC |
| | | GAAGAGGTGATCAGCGAGGACGCCTTCTACAAGTGGGAGAGCAGCA |
| | | AGGACCCCGCCGAGCAGAACGGCAAGGGCGTGGCCCTGAAGAGCGT |
| | | GACCGCCTTCTTCACCTGGCTGCGAGAGGCCGAGGAGGAGAGCGAG |
| | | GACAAC |
| 62 | eIF4G (590-1129) | GEQKYEYKSDQWKPLNLEEKKRYDREFLLGFQFIFASMQKPEGLPH |
| | (aa) | ISDVVLDKANKTPLRPLDPTRLQGINCGPDFTPSFANLGRTTLSTRGPPR |
| | | GGPGGELPRGPAGLGPRRSQQGPRKEPRKIIATVLMTEDIKLNKAEKAW |
| | | KPSSKRTAADKDRGEEDADGSKTQDLFRRVRSILNKLTPQMFQQLMKQ |
| | | VTQLAIDTEERLKGVIDLIFEKAISEPNFSVAYANMCRCLMALKVPTTEK |
| | | PTVTVNFRKLLLNRCQKEFEKDKDDDEVFEKKQKEMDEAATAEERGR |
| | | LKEELEEARDIARRRSLGNIKFIGELFKLKMLTEAIMHDCVVKLLKNHD |
| | | EESLECLCRLLTTIGKDLDFEKAKPRMDQYFNQMEKIIKEKKTSSRIRFM |
| | | LQDVLDLRGSNWVPRRGDQGPKTIDQIHKEAEMEEHREHIKVQQLMA |
| | | KGSDKRRGGPPGPPISRGLPLVDDGGWNTVPISKGSRPIDTSRLTKITKP |
| | | GSIDSNNQLFAPGGRLSWGKGSSGGSGAKPSDAASEAARPATSTLNRFS |
| | | ALQQA |
| 63 | eIF4G (590-1129) | GGCGAGCAGAAGTACGAGTACAAGAGCGACCAGTGGAAGCCTC |
| | (nt) | TGAACCTGGAGGAGAAGAAGAGATACGACAGAGAGTTCCTGCTGGG |
| | | CTTCCAGTTCATATTCGCGAGCATGCAGAAGCCTGAAGGTCTGCCTC |
| | | ACATCAGCGACGTGGTGCTGGACAAGGCCAACAAGACCCCTCTTAG |
| | | ACCTCTGGACCCTACCAGACTGCAGGGCATCAACTGCGGCCCTGACT |
| | | TCACTCCTTCTTTCGCAAACCTGGGCAGAACCACCCTGAGCACCAGA |
| | | GGCCCTCCTAGAGGTGGTCCCGGCGGAGAACTCCCCAGGGGTCCTG |
| | | CCGGCCTGGGCCCTAGACGCTCCCAGCAAGGTCCTAGAAAGGAGCC |
| | | AAGGAAGATCATCGCCACCGTGCTGATGACCGAGGACATCAAGCTG |
| | | AACAAAGCTGAGAAGGCCTGGAAGCCTAGCAGCAAGAGAACCGCC |
| | | GCCGACAAGGACAGAGGCGAGGAGGACGCCGACGGATCCAAGACC |
| | | CAGGACCTGTTCAGAAGAGTGAGAAGCATCCTCAACAAGCTGACCC |
| | | CTCAGATGTTCCAGCAGCTGATGAAGCAGGTGACGCAGCTCGCCATC |
| | | GACACCGAGGAGAGACTGAAGGGCGTGATCGACCTGATCTTTGAGA |
| | | AGGCTATCTCAGAGCCTAACTTCAGCGTGGCCTACGCCAACATGTGC |
| | | CGTTGCCTGATGGCATTGAAGGTGCCAACCACCGAGAAGCCTACTGT |
| | | GACCGTCAATTTCCGTAAACTGCTGCTGAACCGGTGCCAGAAAGAGT |
| | | TCGAGAAGGATAAGGACGACGACGAGGTCTTCGAGAAGAAACAGA |
| | | AAGAAATGGACGAGGCCGCCACCGCAGAGGAAAGGGGCCGATTAA |
| | | AGGAGGAGCTGGAGGAGGCCAGAGACATCGCCAGACGGCGTTCTCT |
| | | GGGCAACATCAAGTTCATAGGTGAGCTGTTCAAGCTAAAGATGCTC |
| | | ACCGAGGCCATAATGCACGACTGCGTGGTGAAGCTACTGAAGAACC |
| | | ACGACGAAGAAAGCCTGGAGTGCCTGTGCAGACTGCTGACCACCAT |
| | | CGGCAAGGACCTGGACTTCGAGAAGGCAAAGCCTCGAATGGACCAG |
| | | TACTTCAACCAGATGGAGAAGATTATCAAGGAGAAGAAGACCAGCA |
| | | GCAGAATCAGATTCATGCTGCAGGACGTACTGGACCTGCGCGGAAG |
| | | CAACTGGGTGCCAAGGAGAGGGGACCAAGGACCAAAGACCATCGA |
| | | CCAGATCCACAAGGAAGCGGAGATGGAGGAGCACAGAGAGCACAT |
| | | AAAGGTGCAGCAGCTTATGGCCAAGGGCAGCGACAAGCGAAGAGG |
| | | CGGCCCGCCCGGACCTCCTATCAGCAGAGGCCTTCCTCTGGTAGACG |
| | | ACGGCGGCTGGAACACCGTGCCTATCTCTAAGGGCTCCAGACCTATC |
| | | GACACTTCCCGTCTTACCAAGATCACCAAGCCAGGATCTATTGACAG |
| | | CAACAACCAGCTGTTCGCCCCAGGAGGAAGACTTAGCTGGGGCAAG |
| | | GGAAGTTCCGGCGGATCCGGCGCCAAGCCTTCCGACGCCGCCAGCG |
| | | AGGCTGCCAGACCTGCCACCAGCACCTTGAACCGCTTTTCCGCTCTG |
| | | CAGCAAGCT |
| 64 | eIF4G3 (aa) | NSQPQTRSPFFQRPQIQPPRATIPNSSPSIRPGAQTPTAVYQANQHIM |
| | | MVNHLPMPYPVPQGPQYCIPQYRHSGPPYVGPPQQYPVQPPGPGPFYPG |
| | | PGPGDFPNAYGTPFYPSQPVYQSAPIIVPTQQQPPPAKREKKTIRIRDPNQ |
| | | GGKDITEEIMSGGGSRNPTPPIGRPTSTPTPPQQLPSQVPEHSPVVYGTVE |
| | | SAHLAASTPVTAASDQKQEEKPKPDPVLKSPSPVLRLVLSGEKKEQEGQ |
| | | TSETTAIVSIAELPLPPSPTTVSSVARSTIAAPTSSALSSQPIFTTAIDDRCE |
| | | LSSPREDTIPIPSLTSCTETSDPLPTNENDDDICKKPCSVAPNDIPLVSSTN |
| | | LINEINGVSEKLSATESIVEIVKQEVLPLTLELEILENPPEEMKLECIPAPIT |
| | | PSTVPSFPPTPPTPPASPPHTPVIVPAAATTVSSPSAAITVQRVLEEDESIRT |
| | | CLSEDAKEIQNKIEVEADGQTEEILDSQNLNSRRSPVPAQIAITVPKTWK |
| | | KPKDRTRTTEEMLEAELELKAEEELSIDKVLESEQDKMSQGFHPERDPS |
| | | DLKKVKAVEENGEEAEPVRNGAESVSEGEGIDANSGSTDSSGDGVTFPF |
| | | KPESWKPTDTEGKKQYDREFLLDFQFMPACIQKPEGLPPISDVVLDKIN |
| | | QPKLPMRTLDPRILPRGPDFTPAFADFGRQTPGGRGVPLLNVGSRRSQP |
| | | GQRREPRKIITVSVKEDVHLKKAENAWKPSQKRDSQADDPENIKTQELF |
| | | RKVRSILNKLTPQMFNQLMKQVSGLTVDTEERLKGVIDLVFEKAIDEPS |
| | | FSVAYANMCRCLVTLKVPMADKPGNTVNFRKLLLNRCQKEFEKDKAD |
| | | DDVFEKKQKELEAASAPEERTRLHDELEEAKDKARRRSIGNIKFIGELFK |
| | | LKMLTEAIMHDCVVKLLKNHDEESLECLCRLLTTIGKDLDFEKAKPRM |
| | | DQYFNQMEKIVKERKTSSRIRFMLQDVIDLRLCNWVSRRADQGPKTIEQ |
| | | IHKEAKIEEQEEQRKVQQLMTKEKRRPGVQRVDEGGWNTVQGAKNSR |
| | | VLDPSKFLKITKPTIDEKIQLVPKAQLGSWGKGSSGGAKASETDALRSSA |
| | | SSLNRFSALQPPAPSGSTPSTPVEFDSRRTLTSRGSMGREKNDKPLPSAT |
| | | ARPNTFMRGGSSKDLLDNQSQEEQRREMLETVKQLTGGVDVERNSTEA |
| | | ERNKTRESAKPEISAMSAHDKAALSEEELERKSKSIIDEFLHINDFKEAM |
| | | QCVEELNAQGLLHVFVRVGVESTLERSQITRDHMGQLLYQLVQSEKLS |
| | | KQDFFKGFSETLELADDMAIDIPHIWLYLAELVTPMLKEGGISMRELTIE |
| | | FSKPLLPVGRAGVLLSEILHLLCKQMSHKKVGALWREADLSWKDFLPE |
| | | GEDVHNFLLEQKLDFIESDSPCSSEALSKKELSAEELYKRLEKLIIEDKAN |
| | | DEQIFDWVEANLDEIQMSSPTFLRALMTAVCKAAIIADSSTFRVDTAVIK |
| | | QRVPILLKYLDSDTEKELQALYALQASIVKLDQPANLLRMFFDCLYDEE |
| | | VISEDAFYKWESSKDPAEQNGKGVALKSVTAFFTWLREAEEESEDN |
| 65 | eIF4G3 (nt) | AGCAACAGCCAACCCCAGACACGGAGCCCCTTCTTCCAGCGGCC |
| | | CCAGATCCAGCCACCACGGGCAACCATCCCCAACAGCAGCCCCAGC |
| | | ATCCGACCCGGCGCACAGACACCCACCGCCGTGTACCAGGCCAACC |
| | | AGCACATCATGATGGTGAACCACCTGCCCATGCCCTACCCCGTGCCA |
| | | CAAGGCCCTCAGTACTGCATCCCACAGTACCGGCACAGCGGTCCTCC |
| | | CTACGTGGGGCCGCCACAGCAGTACCCCGTTCAGCCTCCTGGACCGG |
| | | GACCCTTTTATCCGGGACCTGGACCCGGAGACTTCCCCAACGCCTAC |
| | | GGCACACCCTTCTACCCCAGCCAGCCAGTGTACCAGAGCGCGCCCAT |
| | | CATCGTGCCCACCCAGCAGCAGCCTCCACCCGCCAAGCGGGAGAAG |
| | | AAGACCATCCGGATCCGGGACCCCAACCAGGGCGGCAAGGACATCA |
| | | CCGAGGAGATCATGAGCGGAGGCGGCAGTCGGAACCCCACACCGCC |
| | | CATCGGCAGACCCACCAGCACCCCAACTCCACCACAGCAGCTGCCC |
| | | AGCCAGGTGCCCGAGCACAGCCCCGTGGTGTACGGCACCGTGGAAA |
| | | GCGCCCACCTGGCCGCTAGCACCCCTGTGACCGCCGCCAGCGACCA |
| | | GAAGCAGGAGGAGAAGCCCAAACCCGACCCCGTGCTGAAGTCTCCC |
| | | AGCCCCGTGCTGCGTCTGGTGCTGAGCGGCGAGAAGAAGGAGCAGG |
| | | AGGGCCAGACCAGCGAGACAACCGCCATCGTGAGCATCGCCGAGCT |
| | | GCCACTGCCTCCCAGTCCCACCACCGTGAGCAGCGTGGCCCGGAGC |
| | | ACCATCGCAGCGCCCACAAGCAGCGCCCTGAGCAGCCAGCCCATCT |
| | | TCACCACCGCCATCGACGACCGGTGCGAGCTGAGCAGTCCCCGGGA |
| | | GGACACCATCCCCATCCCCAGCCTGACCAGCTGCACCGAGACCAGC |
| | | GACCCACTGCCCACCAACGAGAACGACGACGACATCTGCAAGAAGC |
| | | CCTGCAGCGTGGCACCCAACGACATCCCGCTGGTGAGCAGCACCAA |
| | | CCTGATCAACGAGATCAACGGCGTTAGCGAGAAACTGTCTGCCACC |
| | | GAGAGCATCGTGGAGATCGTCAAGCAGGAGGTGCTGCCCCTGACCC |
| | | TGGAGCTAGAGATCCTGGAGAACCCTCCAGAGGAGATGAAGCTGGA |
| | | GTGCATCCCCGCCCCAATCACCCCGAGCACCGTGCCCAGCTTTCCTC |
| | | CAACCCCACCCACCCCACCAGCCAGTCCACCTCACACTCCCGTGATC |
| | | GTGCCTGCCGCCGCTACCACCGTGTCTAGCCCCAGCGCCGCCATCAC |
| | | CGTCCAGCGGGTACTGGAGGAGGACGAGAGCATCCGGACCTGCCTG |
| | | AGCGAAGACGCCAAGGAGATCCAGAACAAGATCGAGGTGGAGGCC |
| | | GACGGCCAGACCGAGGAAATCCTGGACAGCCAGAACCTGAATTCCC |
| | | GGCGAAGCCCTGTGCCCGCACAGATCGCCATCACAGTGCCCAAGAC |
| | | CTGGAAGAAGCCCAAGGACCGGACCCGGACCACAGAGGAAATGCTG |
| | | GAGGCCGAGCTGGAGCTGAAGGCCGAGGAAGAGCTGAGCATCGAC |
| | | AAGGTGCTGGAGAGCGAGCAGGACAAGATGAGCCAGGGCTTCCACC |
| | | CTGAGCGGGACCCCAGCGACCTGAAGAAGGTGAAGGCCGTGGAGGA |
| | | GAACGGCGAGGAGGCAGAGCCTGTGCGGAACGGGGCCGAGAGCGT |
| | | GAGCGAGGGCGAGGGCATCGACGCCAACAGCGGCAGCACTGACAG |
| | | CAGCGGCGACGGCGTGACCTTCCCCTTCAAGCCCGAAAGCTGGAAG |
| | | CCCACCGACACCGAAGGAAAGAAGCAGTACGACCGGGAGTTCCTGC |
| | | TGGACTTCCAGTTCATGCCCGCCTGCATCCAGAAACCCGAGGGGCTG |
| | | CCACCAATCAGCGACGTGGTGCTGGACAAGATCAACCAGCCCAAGC |
| | | TGCCCATGCGGACCCTGGATCCTCGGATCCTTCCCAGAGGCCCCGAC |
| | | TTCACTCCCGCCTTCGCCGACTTCGGCAGGCAGACCCCAGGTGGTCG |
| | | GGGTGTGCCCCTGCTGAACGTGGGCAGCCGTCGGAGCCAGCCTGGC |
| | | CAACGGCGGGAACCCCGGAAGATCATCACCGTGAGCGTGAAGGAGG |
| | | ACGTGCATCTGAAGAAGGCCGAGAACGCCTGGAAACCCAGCCAGAA |
| | | GCGGGACAGCCAAGCCGACGACCCCGAGAACATCAAGACCCAGGA |
| | | GCTGTTCCGGAAGGTTCGGAGCATCCTGAACAAGCTGACACCCCAG |
| | | ATGTTCAACCAGCTGATGAAGCAGGTGAGCGGCCTGACCGTGGACA |
| | | CCGAGGAGCGGCTGAAGGGCGTGATCGACCTGGTGTTTGAGAAAGC |
| | | CATTGACGAGCCCAGCTTCAGCGTGGCCTACGCCAACATGTGCCGGT |
| | | GCCTGGTGACCCTGAAGGTGCCCATGGCCGACAAGCCCGGCAACAC |
| | | CGTGAACTTCCGGAAGCTGCTGCTGAACCGGTGCCAGAAGGAGTTC |
| | | GAGAAGGACAAGGCAGACGACGACGTGTTCGAGAAGAAGCAGAAA |
| | | GAGTTAGAGGCCGCAAGCGCCCCTGAGGAGCGGACCCGGCTGCACG |
| | | ACGAGCTGGAGGAGGCCAAAGACAAGGCCCGGCGGAGGAGCATCG |
| | | GCAACATCAAGTTCATCGGCGAGCTGTTCAAGCTGAAGATGCTGACC |
| | | GAGGCCATCATGCACGACTGCGTGGTGAAGCTGCTGAAGAACCACG |
| | | ACGAGGAGAGCCTGGAGTGCCTGTGCCGGCTGCTGACCACCATCGG |
| | | CAAGGACCTGGACTTCGAGAAGGCCAAGCCCCGGATGGACCAGTAC |
| | | TTCAACCAGATGGAGAAGATCGTGAAGGAGCGGAAGACCAGCAGCC |
| | | GGATCCGGTTCATGCTGCAGGACGTTATCGACCTGCGGCTGTGCAAC |
| | | TGGGTGAGCCGGAGGGCTGACCAGGGCCCCAAGACCATCGAGCAGA |
| | | TCCACAAGGAGGCTAAGATCGAGGAGCAGGAAGAGCAGCGGAAGG |
| | | TGCAGCAGCTGATGACCAAAGAGAAGCGGAGGCCTGGCGTGCAGCG |
| | | AGTGGACGAGGGCGGCTGGAACACCGTGCAGGGCGCCAAGAACAG |
| | | CCGGGTGCTGGACCCCAGCAAGTTCCTGAAGATCACCAAGCCCACC |
| | | ATCGACGAGAAGATCCAGCTGGTGCCCAAGGCCCAGCTCGGCAGCT |
| | | GGGGCAAGGGCAGCTCAGGTGGCGCCAAGGCCAGCGAGACCGACG |
| | | CCCTGAGAAGCAGCGCCAGCAGCCTGAACCGGTTCTCTGCCCTGCAG |
| | | CCTCCTGCCCCTAGCGGCTCCACCCCAAGCACACCCGTGGAGTTCGA |
| | | CAGCCGGCGGACCCTGACCAGCCGGGGCTCCATGGGTCGCGAGAAG |
| | | AACGACAAGCCCTTACCCAGCGCAACCGCTCGGCCCAACACCTTCAT |
| | | GCGGGGCGGCAGCTCCAAGGACCTGCTGGACAACCAGAGCCAGGAG |
| | | GAGCAGCGGCGGGAGATGCTGGAGACCGTGAAGCAGCTGACCGGA |
| | | GGCGTGGACGTGGAGCGGAACAGCACCGAGGCCGAGCGGAACAAG |
| | | ACCCGGGAGAGCGCCAAGCCCGAGATCAGCGCCATGAGCGCCCACG |
| | | ACAAGGCCGCCCTGAGCGAGGAGGAGCTGGAACGGAAGAGCAAGA |
| | | GCATCATCGACGAGTTCCTGCACATCAACGACTTCAAGGAGGCCATG |
| | | CAGTGCGTGGAGGAGCTGAACGCCCAGGGCCTGCTGCACGTGTTCG |
| | | TGCGGGTGGGCGTGGAGAGCACCCTGGAGCGGAGCCAGATCACCCG |
| | | GGACCACATGGGCCAGCTGCTGTACCAACTGGTGCAGAGCGAGAAG |
| | | CTGAGCAAGCAGGACTTCTTCAAGGGCTTCAGCGAGACTCTGGAGCT |
| | | GGCCGACGACATGGCCATCGACATTCCCCACATCTGGCTGTACCTGG |
| | | CCGAGCTTGTGACCCCAATGCTGAAGGAGGGCGGCATCAGCATGCG |
| | | GGAGCTGACCATCGAGTTCAGCAAGCCCCTCTTGCCAGTCGGCAGA |
| | | GCAGGAGTGCTGCTGAGCGAGATCCTGCACCTGCTGTGCAAGCAGA |
| | | TGAGCCACAAGAAGGTGGGCGCCCTGTGGAGGGAGGCCGACCTGAG |
| | | CTGGAAGGACTTCCTGCCCGAGGGCGAAGACGTGCACAACTTCTTGC |
| | | TGGAGCAGAAGCTGGACTTCATCGAGAGCGACAGCCCCTGCAGCAG |
| | | CGAGGCCCTGAGCAAGAAGGAACTGAGCGCCGAGGAGCTGTACAAG |
| | | CGGCTGGAGAAGCTGATCATCGAGGACAAGGCCAACGACGAGCAGA |
| | | TCTTCGACTGGGTGGAGGCCAACCTGGACGAGATCCAGATGAGCAG |
| | | CCCCACCTTCCTGCGGGCCCTGATGACCGCCGTGTGCAAGGCCGCCA |
| | | TCATCGCCGACAGCAGCACCTTCCGGGTGGACACCGCCGTGATCAA |
| | | GCAGCGGGTGCCCATCCTGCTGAAGTACCTGGACAGCGACACCGAG |
| | | AAGGAGCTGCAGGCCCTGTACGCCCTGCAGGCCAGCATCGTGAAGC |
| | | TGGACCAGCCCGCCAACCTGCTGCGGATGTTCTTCGACTGCCTGTAC |
| | | GACGAAGAGGTGATCAGCGAGGACGCCTTCTACAAGTGGGAGAGCA |
| | | GCAAGGACCCCGCCGAGCAGAACGGCAAGGGCGTGGCCCTGAAGA |
| | | GCGTGACCGCCTTCTTCACCTGGCTGCGAGAGGCCGAGGAGGAGAG |
| | | CGAGGACAAC |
| 66 | eIF4G3-mid (aa) | RTLDPRILPRGPDFTPAFADFGRQTPGGRGVPLLNVGSRRSQPGQRR |
| | | EPRKIITVSVKEDVHLKKAENAWKPSQKRDSQADDPENIKTQELFRKVR |
| | | SILNKLTPQMFNQLMKQVSGLTVDTEERLKGVIDLVFEKAIDEPSFSVA |
| | | YANMCRCLVTLKVPMADKPGNTVNFRKLLLNRCQKEFEKDKADDDVF |
| | | EKKQKELEAASAPEERTRLHDELEEAKDKARRRSIGNIKFIGELFKLKM |
| | | LTEAIMHDCVVKLLKNHDEESLECLCRLLTTIGKDLDFEKAKPRMDQY |
| | | FNQMEKIVKERKTSSRIRFMLQDVIDLRLCNWVSRRADQGPKTIEQIHK |
| | | EAKIEEQEEQRKVQQLMTKEKRRPGVQRVDEGGWNTVQGAKNSRVLD |
| | | PSKFLKITKPTIDEKIQLVPKAQLGSWGKGSSG |
| 67 | eIF4G3-mid (nt) | AGCCGGACCCTGGATCCTCGGATCCTTCCCAGAGGCCCCGACTT |
| | | CACTCCCGCCTTCGCCGACTTCGGCAGGCAGACCCCAGGTGGTCGGG |
| | | GTGTGCCCCTGCTGAACGTGGGCAGCCGTCGGAGCCAGCCTGGCCA |
| | | ACGGCGGGAACCCCGGAAGATCATCACCGTGAGCGTGAAGGAGGAC |
| | | GTGCATCTGAAGAAGGCCGAGAACGCCTGGAAACCCAGCCAGAAGC |
| | | GGGACAGCCAAGCCGACGACCCCGAGAACATCAAGACCCAGGAGCT |
| | | GTTCCGGAAGGTTCGGAGCATCCTGAACAAGCTGACACCCCAGATG |
| | | TTCAACCAGCTGATGAAGCAGGTGAGCGGCCTGACCGTGGACACCG |
| | | AGGAGCGGCTGAAGGGCGTGATCGACCTGGTGTTTGAGAAAGCCAT |
| | | TGACGAGCCCAGCTTCAGCGTGGCCTACGCCAACATGTGCCGGTGCC |
| | | TGGTGACCCTGAAGGTGCCCATGGCCGACAAGCCCGGCAACACCGT |
| | | GAACTTCCGGAAGCTGCTGCTGAACCGGTGCCAGAAGGAGTTCGAG |
| | | AAGGACAAGGCAGACGACGACGTGTTCGAGAAGAAGCAGAAAGAG |
| | | TTAGAGGCCGCAAGCGCCCCTGAGGAGCGGACCCGGCTGCACGACG |
| | | AGCTGGAGGAGGCCAAAGACAAGGCCCGGCGGAGGAGCATCGGCA |
| | | ACATCAAGTTCATCGGCGAGCTGTTCAAGCTGAAGATGCTGACCGA |
| | | GGCCATCATGCACGACTGCGTGGTGAAGCTGCTGAAGAACCACGAC |
| | | GAGGAGAGCCTGGAGTGCCTGTGCCGGCTGCTGACCACCATCGGCA |
| | | AGGACCTGGACTTCGAGAAGGCCAAGCCCCGGATGGACCAGTACTT |
| | | CAACCAGATGGAGAAGATCGTGAAGGAGCGGAAGACCAGCAGCCG |
| | | GATCCGGTTCATGCTGCAGGACGTTATCGACCTGCGGCTGTGCAACT |
| | | GGGTGAGCCGGAGGGCTGACCAGGGCCCCAAGACCATCGAGCAGAT |
| | | CCACAAGGAGGCTAAGATCGAGGAGCAGGAAGAGCAGCGGAAGGT |
| | | GCAGCAGCTGATGACCAAAGAGAAGCGGAGGCCTGGCGTGCAGCGA |
| | | GTGGACGAGGGCGGCTGGAACACCGTGCAGGGCGCCAAGAACAGCC |
| | | GGGTGCTGGACCCCAGCAAGTTCCTGAAGATCACCAAGCCCACCAT |
| | | CGACGAGAAGATCCAGCTGGTGCCCAAGGCCCAGCTCGGCAGCTGG |
| | | GGCAAGGGCAGCTCAGGT |
| 68 | MBP-eIF4G- | MASNFTQFVLVDNGGTGDVTVAPSNFANGIAEWISSNSRSQAYKV |
| | mid2mut (aa) | TCSVRQSSAQNRKYTIKVEVPKGAWRSYLNMELTIPIFATNSDCELIVK |
| | | AMQGLLKDGNPIPSAIAANSGIYGGGGSDPTRLQGINCGPDFTPSFANLG |
| | | RTTLSTRGPPRGGPGGELPRGPAGLGPRRSQQGPRKEPRKIIATVLMTED |
| | | IKLNKAEKAWKPSSKRTAADKDRGEEDADGSKTQDLFRRVRSILNKLT |
| | | PQMFQQLMKQVTQLAIDTEERLKGVIDLIFEKAISEPNFSVAYANMCRC |
| | | LMALKVPTTEKPTVTVNFRKLLLNRCQKEFEKDKDDDEVFEKKQKEM |
| | | DEAATAEERGRLKEELEEARDIARRRSLGNIKAIGELFKLKMLTEAIMH |
| | | DCVVKLLKNHDEESLECLCRLLTTIGKDLDFEKAKPRMDQYFNQMEKII |
| | | KEKKTSSRIRFMLQDVLDLRGSNWVPRRGDQGPKTIDQIHKEAEMEEH |
| | | REHIKVQQLMAKGSDKRRGGPPGPPISRGLPLVDDGGWNTVPISKGSRP |
| | | IDTSRLTKITKPGSIDSNNQLFAPGGRLSWGKGSSGGSGAKPSDAASEAA |
| | | RPATSTLNRFSALQQAV |
| 69 | MBP-eIF4G- | ATGGCCAGCAACTTCACCCAGTTCGTGCTGGTGGACAACGGCGG |
| | mid2mut (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCGA |
| | | CCCTACCAGACTGCAGGGCATCAACTGCGGCCCTGACTTCACTCCTT |
| | | CTTTCGCAAACCTGGGCAGAACCACCCTGAGCACCAGAGGCCCTCCT |
| | | AGAGGTGGTCCCGGCGGAGAACTCCCCAGGGGTCCTGCCGGCCTGG |
| | | GCCCTAGACGCTCCCAGCAAGGTCCTAGAAAGGAGCCAAGGAAGAT |
| | | CATCGCCACCGTGCTGATGACCGAGGACATCAAGCTGAACAAAGCT |
| | | GAGAAGGCCTGGAAGCCTAGCAGCAAGAGAACCGCCGCCGACAAG |
| | | GACAGAGGCGAGGAGGACGCCGACGGATCCAAGACCCAGGACCTGT |
| | | TCAGAAGAGTGAGAAGCATCCTCAACAAGCTGACCCCTCAGATGTT |
| | | CCAGCAGCTGATGAAGCAGGTGACGCAGCTCGCCATCGACACCGAG |
| | | GAGAGACTGAAGGGCGTGATCGACCTGATCTTTGAGAAGGCTATCT |
| | | CAGAGCCTAACTTCAGCGTGGCCTACGCCAACATGTGCCGTTGCCTG |
| | | ATGGCATTGAAGGTGCCAACCACCGAGAAGCCTACTGTGACCGTCA |
| | | ATTTCCGTAAACTGCTGCTGAACCGGTGCCAGAAAGAGTTCGAGAA |
| | | GGATAAGGACGACGACGAGGTCTTCGAGAAGAAACAGAAAGAAAT |
| | | GGACGAGGCCGCCACCGCAGAGGAAAGGGGCCGATTAAAGGAGGA |
| | | GCTGGAGGAGGCCAGAGACATCGCCAGACGGCGTTCTCTGGGCAAC |
| | | ATCAAGGCGATAGGTGAGCTGTTCAAGCTAAAGATGCTCACCGAGG |
| | | CCATAATGCACGACTGCGTGGTGAAGCTACTGAAGAACCACGACGA |
| | | AGAAAGCCTGGAGTGCCTGTGCAGACTGCTGACCACCATCGGCAAG |
| | | GACCTGGACTTCGAGAAGGCAAAGCCTCGAATGGACCAGTACTTCA |
| | | ACCAGATGGAGAAGATTATCAAGGAGAAGAAGACCAGCAGCAGAA |
| | | TCAGATTCATGCTGCAGGACGTACTGGACCTGCGCGGAAGCAACTG |
| | | GGTGCCAAGGAGAGGGGACCAAGGACCAAAGACCATCGACCAGAT |
| | | CCACAAGGAAGCGGAGATGGAGGAGCACAGAGAGCACATAAAGGT |
| | | GCAGCAGCTTATGGCCAAGGGCAGCGACAAGCGAAGAGGCGGCCCG |
| | | CCCGGACCTCCTATCAGCAGAGGCCTTCCTCTGGTAGACGACGGCGG |
| | | CTGGAACACCGTGCCTATCTCTAAGGGCTCCAGACCTATCGACACTT |
| | | CCCGTCTTACCAAGATCACCAAGCCAGGATCTATTGACAGCAACAAC |
| | | CAGCTGTTCGCCCCAGGAGGAAGACTTAGCTGGGGCAAGGGAAGTT |
| | | CCGGCGGATCCGGCGCCAAGCCTTCCGACGCCGCCAGCGAGGCTGC |
| | | CAGACCTGCCACCAGCACCTTGAACCGCTTTTCCGCTCTGCAGCAAG |
| | | CTGTG |
| 70 | MBP-eIF4G | MASNFTQFVLVDNGGTGDVTVAPSNFANGIAEWISSNSRSQAYKV |
| | (590-1129) (aa) | TCSVRQSSAQNRKYTIKVEVPKGAWRSYLNMELTIPIFATNSDCELIVK |
| | | AMQGLLKDGNPIPSAIAANSGIYGGGGSGEQKYEYKSDQWKPLNLEEK |
| | | KRYDREFLLGFQFIFASMQKPEGLPHISDVVLDKANKTPLRPLDPTRLQ |
| | | GINCGPDFTPSFANLGRTTLSTRGPPRGGPGGELPRGPAGLGPRRSQQGP |
| | | RKEPRKIIATVLMTEDIKLNKAEKAWKPSSKRTAADKDRGEEDADGSK |
| | | TQDLFRRVRSILNKLTPQMFQQLMKQVTQLAIDTEERLKGVIDLIFEKAI |
| | | SEPNFSVAYANMCRCLMALKVPTTEKPTVTVNFRKLLLNRCQKEFEKD |
| | | KDDDEVFEKKQKEMDEAATAEERGRLKEELEEARDIARRRSLGNIKFIG |
| | | ELFKLKMLTEAIMHDCVVKLLKNHDEESLECLCRLLTTIGKDLDFEKAK |
| | | PRMDQYFNQMEKIIKEKKTSSRIRFMLQDVLDLRGSNWVPRRGDQGPK |
| | | TIDQIHKEAEMEEHREHIKVQQLMAKGSDKRRGGPPGPPISRGLPLVDD |
| | | GGWNTVPISKGSRPIDTSRLTKITKPGSIDSNNQLFAPGGRLSWGKGSSG |
| | | GSGAKPSDAASEAARPATSTLNRFSALQQA |
| 71 | MBP-eIF4G | ATGGCCAGCAACTTCACCCAGTTCGTGCTGGTGGACAACGGCGG |
| | (590-1129) (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCGG |
| | | CGAGCAGAAGTACGAGTACAAGAGCGACCAGTGGAAGCCTCTGAAC |
| | | CTGGAGGAGAAGAAGAGATACGACAGAGAGTTCCTGCTGGGCTTCC |
| | | AGTTCATATTCGCGAGCATGCAGAAGCCTGAAGGTCTGCCTCACATC |
| | | AGCGACGTGGTGCTGGACAAGGCCAACAAGACCCCTCTTAGACCTC |
| | | TGGACCCTACCAGACTGCAGGGCATCAACTGCGGCCCTGACTTCACT |
| | | CCTTCTTTCGCAAACCTGGGCAGAACCACCCTGAGCACCAGAGGCCC |
| | | TCCTAGAGGTGGTCCCGGCGGAGAACTCCCCAGGGGTCCTGCCGGC |
| | | CTGGGCCCTAGACGCTCCCAGCAAGGTCCTAGAAAGGAGCCAAGGA |
| | | AGATCATCGCCACCGTGCTGATGACCGAGGACATCAAGCTGAACAA |
| | | AGCTGAGAAGGCCTGGAAGCCTAGCAGCAAGAGAACCGCCGCCGAC |
| | | AAGGACAGAGGCGAGGAGGACGCCGACGGATCCAAGACCCAGGAC |
| | | CTGTTCAGAAGAGTGAGAAGCATCCTCAACAAGCTGACCCCTCAGA |
| | | TGTTCCAGCAGCTGATGAAGCAGGTGACGCAGCTCGCCATCGACAC |
| | | CGAGGAGAGACTGAAGGGCGTGATCGACCTGATCTTTGAGAAGGCT |
| | | ATCTCAGAGCCTAACTTCAGCGTGGCCTACGCCAACATGTGCCGTTG |
| | | CCTGATGGCATTGAAGGTGCCAACCACCGAGAAGCCTACTGTGACC |
| | | GTCAATTTCCGTAAACTGCTGCTGAACCGGTGCCAGAAAGAGTTCGA |
| | | GAAGGATAAGGACGACGACGAGGTCTTCGAGAAGAAACAGAAAGA |
| | | AATGGACGAGGCCGCCACCGCAGAGGAAAGGGGCCGATTAAAGGA |
| | | GGAGCTGGAGGAGGCCAGAGACATCGCCAGACGGCGTTCTCTGGGC |
| | | AACATCAAGTTCATAGGTGAGCTGTTCAAGCTAAAGATGCTCACCGA |
| | | GGCCATAATGCACGACTGCGTGGTGAAGCTACTGAAGAACCACGAC |
| | | GAAGAAAGCCTGGAGTGCCTGTGCAGACTGCTGACCACCATCGGCA |
| | | AGGACCTGGACTTCGAGAAGGCAAAGCCTCGAATGGACCAGTACTT |
| | | CAACCAGATGGAGAAGATTATCAAGGAGAAGAAGACCAGCAGCAG |
| | | AATCAGATTCATGCTGCAGGACGTACTGGACCTGCGCGGAAGCAAC |
| | | TGGGTGCCAAGGAGAGGGGACCAAGGACCAAAGACCATCGACCAG |
| | | ATCCACAAGGAAGCGGAGATGGAGGAGCACAGAGAGCACATAAAG |
| | | GTGCAGCAGCTTATGGCCAAGGGCAGCGACAAGCGAAGAGGCGGCC |
| | | CGCCCGGACCTCCTATCAGCAGAGGCCTTCCTCTGGTAGACGACGGC |
| | | GGCTGGAACACCGTGCCTATCTCTAAGGGCTCCAGACCTATCGACAC |
| | | TTCCCGTCTTACCAAGATCACCAAGCCAGGATCTATTGACAGCAACA |
| | | ACCAGCTGTTCGCCCCAGGAGGAAGACTTAGCTGGGGCAAGGGAAG |
| | | TTCCGGCGGATCCGGCGCCAAGCCTTCCGACGCCGCCAGCGAGGCT |
| | | GCCAGACCTGCCACCAGCACCTTGAACCGCTTTTCCGCTCTGCAGCA |
| | | AGCT |
| 72 | MBP-eIF4G3 (aa) | MASNFTQFVLVDNGGTGDVTVAPSNFANGIAEWISSNSRSQAYKV |
| | | TCSVRQSSAQNRKYTIKVEVPKGAWRSYLNMELTIPIFATNSDCELIVK |
| | | AMQGLLKDGNPIPSAIAANSGIYGGGGSNSQPQTRSPFFQRPQIQPPRATI |
| | | PNSSPSIRPGAQTPTAVYQANQHIMMVNHLPMPYPVPQGPQYCIPQYRH |
| | | SGPPYVGPPQQYPVQPPGPGPFYPGPGPGDFPNAYGTPFYPSQPVYQSAP |
| | | IIVPTQQQPPPAKREKKTIRIRDPNQGGKDITEEIMSGGGSRNPTPPIGRPT |
| | | STPTPPQQLPSQVPEHSPVVYGTVESAHLAASTPVTAASDQKQEEKPKP |
| | | DPVLKSPSPVLRLVLSGEKKEQEGQTSETTAIVSIAELPLPPSPTTVSSVA |
| | | RSTIAAPTSSALSSQPIFTTAIDDRCELSSPREDTIPIPSLTSCTETSDPLPTN |
| | | ENDDDICKKPCSVAPNDIPLVSSTNLINEINGVSEKLSATESIVEIVKQEV |
| | | LPLTLELEILENPPEEMKLECIPAPITPSTVPSFPPTPPTPPASPPHTPVIVPA |
| | | AATTVSSPSAAITVQRVLEEDESIRTCLSEDAKEIQNKIEVEADGQTEEIL |
| | | DSQNLNSRRSPVPAQIAITVPKTWKKPKDRTRTTEEMLEAELELKAEEE |
| | | LSIDKVLESEQDKMSQGFHPERDPSDLKKVKAVEENGEEAEPVRNGAE |
| | | SVSEGEGIDANSGSTDSSGDGVTFPFKPESWKPTDTEGKKQYDREFLLD |
| | | FQFMPACIQKPEGLPPISDVVLDKINQPKLPMRTLDPRILPRGPDFTPAFA |
| | | DFGRQTPGGRGVPLLNVGSRRSQPGQRREPRKIITVSVKEDVHLKKAEN |
| | | AWKPSQKRDSQADDPENIKTQELFRKVRSILNKLTPQMFNQLMKQVSG |
| | | LTVDTEERLKGVIDLVFEKAIDEPSFSVAYANMCRCLVTLKVPMADKP |
| | | GNTVNFRKLLLNRCQKEFEKDKADDDVFEKKQKELEAASAPEERTRLH |
| | | DELEEAKDKARRRSIGNIKFIGELFKLKMLTEAIMHDCVVKLLKNHDEE |
| | | SLECLCRLLTTIGKDLDFEKAKPRMDQYFNQMEKIVKERKTSSRIRFML |
| | | QDVIDLRLCNWVSRRADQGPKTIEQIHKEAKIEEQEEQRKVQQLMTKE |
| | | KRRPGVQRVDEGGWNTVQGAKNSRVLDPSKFLKITKPTIDEKIQLVPKA |
| | | QLGSWGKGSSGGAKASETDALRSSASSLNRFSALQPPAPSGSTPSTPVEF |
| | | DSRRTLTSRGSMGREKNDKPLPSATARPNTFMRGGSSKDLLDNQSQEE |
| | | QRREMLETVKQLTGGVDVERNSTEAERNKTRESAKPEISAMSAHDKAA |
| | | LSEEELERKSKSIIDEFLHINDFKEAMQCVEELNAQGLLHVFVRVGVEST |
| | | LERSQITRDHMGQLLYQLVQSEKLSKQDFFKGFSETLELADDMAIDIPHI |
| | | WLYLAELVTPMLKEGGISMRELTIEFSKPLLPVGRAGVLLSEILHLLCKQ |
| | | MSHKKVGALWREADLSWKDFLPEGEDVHNFLLEQKLDFIESDSPCSSE |
| | | ALSKKELSAEELYKRLEKLIIEDKANDEQIFDWVEANLDEIQMSSPTFLR |
| | | ALMTAVCKAAIIADSSTFRVDTAVIKQRVPILLKYLDSDTEKELQALYA |
| | | LQASIVKLDQPANLLRMFFDCLYDEEVISEDAFYKWESSKDPAEQNGK |
| | | GVALKSVTAFFTWLREAEEESEDN |
| 73 | MBP-eIF4G3 (nt) | ATGGCCAGCAACTTCACCCAGTTCGTGCTGGTGGACAACGGCGG |
| | | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCAA |
| | | CAGCCAACCCCAGACACGGAGCCCCTTCTTCCAGCGGCCCCAGATCC |
| | | AGCCACCACGGGCAACCATCCCCAACAGCAGCCCCAGCATCCGACC |
| | | CGGCGCACAGACACCCACCGCCGTGTACCAGGCCAACCAGCACATC |
| | | ATGATGGTGAACCACCTGCCCATGCCCTACCCCGTGCCACAAGGCCC |
| | | TCAGTACTGCATCCCACAGTACCGGCACAGCGGTCCTCCCTACGTGG |
| | | GGCCGCCACAGCAGTACCCCGTTCAGCCTCCTGGACCGGGACCCTTT |
| | | TATCCGGGACCTGGACCCGGAGACTTCCCCAACGCCTACGGCACACC |
| | | CTTCTACCCCAGCCAGCCAGTGTACCAGAGCGCGCCCATCATCGTGC |
| | | CCACCCAGCAGCAGCCTCCACCCGCCAAGCGGGAGAAGAAGACCAT |
| | | CCGGATCCGGGACCCCAACCAGGGCGGCAAGGACATCACCGAGGAG |
| | | ATCATGAGCGGAGGCGGCAGTCGGAACCCCACACCGCCCATCGGCA |
| | | GACCCACCAGCACCCCAACTCCACCACAGCAGCTGCCCAGCCAGGT |
| | | GCCCGAGCACAGCCCCGTGGTGTACGGCACCGTGGAAAGCGCCCAC |
| | | CTGGCCGCTAGCACCCCTGTGACCGCCGCCAGCGACCAGAAGCAGG |
| | | AGGAGAAGCCCAAACCCGACCCCGTGCTGAAGTCTCCCAGCCCCGT |
| | | GCTGCGTCTGGTGCTGAGCGGCGAGAAGAAGGAGCAGGAGGGCCAG |
| | | ACCAGCGAGACAACCGCCATCGTGAGCATCGCCGAGCTGCCACTGC |
| | | CTCCCAGTCCCACCACCGTGAGCAGCGTGGCCCGGAGCACCATCGC |
| | | AGCGCCCACAAGCAGCGCCCTGAGCAGCCAGCCCATCTTCACCACC |
| | | GCCATCGACGACCGGTGCGAGCTGAGCAGTCCCCGGGAGGACACCA |
| | | TCCCCATCCCCAGCCTGACCAGCTGCACCGAGACCAGCGACCCACTG |
| | | CCCACCAACGAGAACGACGACGACATCTGCAAGAAGCCCTGCAGCG |
| | | TGGCACCCAACGACATCCCGCTGGTGAGCAGCACCAACCTGATCAA |
| | | CGAGATCAACGGCGTTAGCGAGAAACTGTCTGCCACCGAGAGCATC |
| | | GTGGAGATCGTCAAGCAGGAGGTGCTGCCCCTGACCCTGGAGCTAG |
| | | AGATCCTGGAGAACCCTCCAGAGGAGATGAAGCTGGAGTGCATCCC |
| | | CGCCCCAATCACCCCGAGCACCGTGCCCAGCTTTCCTCCAACCCCAC |
| | | CCACCCCACCAGCCAGTCCACCTCACACTCCCGTGATCGTGCCTGCC |
| | | GCCGCTACCACCGTGTCTAGCCCCAGCGCCGCCATCACCGTCCAGCG |
| | | GGTACTGGAGGAGGACGAGAGCATCCGGACCTGCCTGAGCGAAGAC |
| | | GCCAAGGAGATCCAGAACAAGATCGAGGTGGAGGCCGACGGCCAG |
| | | ACCGAGGAAATCCTGGACAGCCAGAACCTGAATTCCCGGCGAAGCC |
| | | CTGTGCCCGCACAGATCGCCATCACAGTGCCCAAGACCTGGAAGAA |
| | | GCCCAAGGACCGGACCCGGACCACAGAGGAAATGCTGGAGGCCGA |
| | | GCTGGAGCTGAAGGCCGAGGAAGAGCTGAGCATCGACAAGGTGCTG |
| | | GAGAGCGAGCAGGACAAGATGAGCCAGGGCTTCCACCCTGAGCGGG |
| | | ACCCCAGCGACCTGAAGAAGGTGAAGGCCGTGGAGGAGAACGGCG |
| | | AGGAGGCAGAGCCTGTGCGGAACGGGGCCGAGAGCGTGAGCGAGG |
| | | GCGAGGGCATCGACGCCAACAGCGGCAGCACTGACAGCAGCGGCGA |
| | | CGGCGTGACCTTCCCCTTCAAGCCCGAAAGCTGGAAGCCCACCGAC |
| | | ACCGAAGGAAAGAAGCAGTACGACCGGGAGTTCCTGCTGGACTTCC |
| | | AGTTCATGCCCGCCTGCATCCAGAAACCCGAGGGGCTGCCACCAATC |
| | | AGCGACGTGGTGCTGGACAAGATCAACCAGCCCAAGCTGCCCATGC |
| | | GGACCCTGGATCCTCGGATCCTTCCCAGAGGCCCCGACTTCACTCCC |
| | | GCCTTCGCCGACTTCGGCAGGCAGACCCCAGGTGGTCGGGGTGTGCC |
| | | CCTGCTGAACGTGGGCAGCCGTCGGAGCCAGCCTGGCCAACGGCGG |
| | | GAACCCCGGAAGATCATCACCGTGAGCGTGAAGGAGGACGTGCATC |
| | | TGAAGAAGGCCGAGAACGCCTGGAAACCCAGCCAGAAGCGGGACA |
| | | GCCAAGCCGACGACCCCGAGAACATCAAGACCCAGGAGCTGTTCCG |
| | | GAAGGTTCGGAGCATCCTGAACAAGCTGACACCCCAGATGTTCAAC |
| | | CAGCTGATGAAGCAGGTGAGCGGCCTGACCGTGGACACCGAGGAGC |
| | | GGCTGAAGGGCGTGATCGACCTGGTGTTTGAGAAAGCCATTGACGA |
| | | GCCCAGCTTCAGCGTGGCCTACGCCAACATGTGCCGGTGCCTGGTGA |
| | | CCCTGAAGGTGCCCATGGCCGACAAGCCCGGCAACACCGTGAACTT |
| | | CCGGAAGCTGCTGCTGAACCGGTGCCAGAAGGAGTTCGAGAAGGAC |
| | | AAGGCAGACGACGACGTGTTCGAGAAGAAGCAGAAAGAGTTAGAG |
| | | GCCGCAAGCGCCCCTGAGGAGCGGACCCGGCTGCACGACGAGCTGG |
| | | AGGAGGCCAAAGACAAGGCCCGGCGGAGGAGCATCGGCAACATCA |
| | | AGTTCATCGGCGAGCTGTTCAAGCTGAAGATGCTGACCGAGGCCATC |
| | | ATGCACGACTGCGTGGTGAAGCTGCTGAAGAACCACGACGAGGAGA |
| | | GCCTGGAGTGCCTGTGCCGGCTGCTGACCACCATCGGCAAGGACCTG |
| | | GACTTCGAGAAGGCCAAGCCCCGGATGGACCAGTACTTCAACCAGA |
| | | TGGAGAAGATCGTGAAGGAGCGGAAGACCAGCAGCCGGATCCGGTT |
| | | CATGCTGCAGGACGTTATCGACCTGCGGCTGTGCAACTGGGTGAGCC |
| | | GGAGGGCTGACCAGGGCCCCAAGACCATCGAGCAGATCCACAAGGA |
| | | GGCTAAGATCGAGGAGCAGGAAGAGCAGCGGAAGGTGCAGCAGCT |
| | | GATGACCAAAGAGAAGCGGAGGCCTGGCGTGCAGCGAGTGGACGA |
| | | GGGCGGCTGGAACACCGTGCAGGGCGCCAAGAACAGCCGGGTGCTG |
| | | GACCCCAGCAAGTTCCTGAAGATCACCAAGCCCACCATCGACGAGA |
| | | AGATCCAGCTGGTGCCCAAGGCCCAGCTCGGCAGCTGGGGCAAGGG |
| | | CAGCTCAGGTGGCGCCAAGGCCAGCGAGACCGACGCCCTGAGAAGC |
| | | AGCGCCAGCAGCCTGAACCGGTTCTCTGCCCTGCAGCCTCCTGCCCC |
| | | TAGCGGCTCCACCCCAAGCACACCCGTGGAGTTCGACAGCCGGCGG |
| | | ACCCTGACCAGCCGGGGCTCCATGGGTCGCGAGAAGAACGACAAGC |
| | | CCTTACCCAGCGCAACCGCTCGGCCCAACACCTTCATGCGGGGCGGC |
| | | AGCTCCAAGGACCTGCTGGACAACCAGAGCCAGGAGGAGCAGCGGC |
| | | GGGAGATGCTGGAGACCGTGAAGCAGCTGACCGGAGGCGTGGACGT |
| | | GGAGCGGAACAGCACCGAGGCCGAGCGGAACAAGACCCGGGAGAG |
| | | CGCCAAGCCCGAGATCAGCGCCATGAGCGCCCACGACAAGGCCGCC |
| | | CTGAGCGAGGAGGAGCTGGAACGGAAGAGCAAGAGCATCATCGAC |
| | | GAGTTCCTGCACATCAACGACTTCAAGGAGGCCATGCAGTGCGTGG |
| | | AGGAGCTGAACGCCCAGGGCCTGCTGCACGTGTTCGTGCGGGTGGG |
| | | CGTGGAGAGCACCCTGGAGCGGAGCCAGATCACCCGGGACCACATG |
| | | GGCCAGCTGCTGTACCAACTGGTGCAGAGCGAGAAGCTGAGCAAGC |
| | | AGGACTTCTTCAAGGGCTTCAGCGAGACTCTGGAGCTGGCCGACGA |
| | | CATGGCCATCGACATTCCCCACATCTGGCTGTACCTGGCCGAGCTTG |
| | | TGACCCCAATGCTGAAGGAGGGCGGCATCAGCATGCGGGAGCTGAC |
| | | CATCGAGTTCAGCAAGCCCCTCTTGCCAGTCGGCAGAGCAGGAGTG |
| | | CTGCTGAGCGAGATCCTGCACCTGCTGTGCAAGCAGATGAGCCACA |
| | | AGAAGGTGGGCGCCCTGTGGAGGGAGGCCGACCTGAGCTGGAAGGA |
| | | CTTCCTGCCCGAGGGCGAAGACGTGCACAACTTCTTGCTGGAGCAGA |
| | | AGCTGGACTTCATCGAGAGCGACAGCCCCTGCAGCAGCGAGGCCCT |
| | | GAGCAAGAAGGAACTGAGCGCCGAGGAGCTGTACAAGCGGCTGGA |
| | | GAAGCTGATCATCGAGGACAAGGCCAACGACGAGCAGATCTTCGAC |
| | | TGGGTGGAGGCCAACCTGGACGAGATCCAGATGAGCAGCCCCACCT |
| | | TCCTGCGGGCCCTGATGACCGCCGTGTGCAAGGCCGCCATCATCGCC |
| | | GACAGCAGCACCTTCCGGGTGGACACCGCCGTGATCAAGCAGCGGG |
| | | TGCCCATCCTGCTGAAGTACCTGGACAGCGACACCGAGAAGGAGCT |
| | | GCAGGCCCTGTACGCCCTGCAGGCCAGCATCGTGAAGCTGGACCAG |
| | | CCCGCCAACCTGCTGCGGATGTTCTTCGACTGCCTGTACGACGAAGA |
| | | GGTGATCAGCGAGGACGCCTTCTACAAGTGGGAGAGCAGCAAGGAC |
| | | CCCGCCGAGCAGAACGGCAAGGGCGTGGCCCTGAAGAGCGTGACCG |
| | | CCTTCTTCACCTGGCTGCGAGAGGCCGAGGAGGAGAGCGAGGACAA |
| | | C |
| 74 | MBP-eIF4G3-mid | MASNFTQFVLVDNGGTGDVTVAPSNFANGIAEWISSNSRSQAYKV |
| | (aa) | TCSVRQSSAQNRKYTIKVEVPKGAWRSYLNMELTIPIFATNSDCELIVK |
| | | AMQGLLKDGNPIPSAIAANSGIYGGGGSRTLDPRILPRGPDFTPAFADFG |
| | | RQTPGGRGVPLLNVGSRRSQPGQRREPRKIITVSVKEDVHLKKAENAW |
| | | KPSQKRDSQADDPENIKTQELFRKVRSILNKLTPQMFNQLMKQVSGLTV |
| | | DTEERLKGVIDLVFEKAIDEPSFSVAYANMCRCLVTLKVPMADKPGNT |
| | | VNFRKLLLNRCQKEFEKDKADDDVFEKKQKELEAASAPEERTRLHDEL |
| | | EEAKDKARRRSIGNIKFIGELFKLKMLTEAIMHDCVVKLLKNHDEESLE |
| | | CLCRLLTTIGKDLDFEKAKPRMDQYFNQMEKIVKERKTSSRIRFMLQDV |
| | | IDLRLCNWVSRRADQGPKTIEQIHKEAKIEEQEEQRKVQQLMTKEKRRP |
| | | GVQRVDEGGWNTVQGAKNSRVLDPSKFLKITKPTIDEKIQLVPKAQLGS |
| | | WGKGSSG |
| 75 | MBP-eIF4G3-mid | ATGGCCAGCAACTTCACCCAGTTCGTGCTGGTGGACAACGGCGG |
| | (nt) | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCCG |
| | | GACCCTGGATCCTCGGATCCTTCCCAGAGGCCCCGACTTCACTCCCG |
| | | CCTTCGCCGACTTCGGCAGGCAGACCCCAGGTGGTCGGGGTGTGCCC |
| | | CTGCTGAACGTGGGCAGCCGTCGGAGCCAGCCTGGCCAACGGCGGG |
| | | AACCCCGGAAGATCATCACCGTGAGCGTGAAGGAGGACGTGCATCT |
| | | GAAGAAGGCCGAGAACGCCTGGAAACCCAGCCAGAAGCGGGACAG |
| | | CCAAGCCGACGACCCCGAGAACATCAAGACCCAGGAGCTGTTCCGG |
| | | AAGGTTCGGAGCATCCTGAACAAGCTGACACCCCAGATGTTCAACC |
| | | AGCTGATGAAGCAGGTGAGCGGCCTGACCGTGGACACCGAGGAGCG |
| | | GCTGAAGGGCGTGATCGACCTGGTGTTTGAGAAAGCCATTGACGAG |
| | | CCCAGCTTCAGCGTGGCCTACGCCAACATGTGCCGGTGCCTGGTGAC |
| | | CCTGAAGGTGCCCATGGCCGACAAGCCCGGCAACACCGTGAACTTC |
| | | CGGAAGCTGCTGCTGAACCGGTGCCAGAAGGAGTTCGAGAAGGACA |
| | | AGGCAGACGACGACGTGTTCGAGAAGAAGCAGAAAGAGTTAGAGG |
| | | CCGCAAGCGCCCCTGAGGAGCGGACCCGGCTGCACGACGAGCTGGA |
| | | GGAGGCCAAAGACAAGGCCCGGCGGAGGAGCATCGGCAACATCAA |
| | | GTTCATCGGCGAGCTGTTCAAGCTGAAGATGCTGACCGAGGCCATCA |
| | | TGCACGACTGCGTGGTGAAGCTGCTGAAGAACCACGACGAGGAGAG |
| | | CCTGGAGTGCCTGTGCCGGCTGCTGACCACCATCGGCAAGGACCTGG |
| | | ACTTCGAGAAGGCCAAGCCCCGGATGGACCAGTACTTCAACCAGAT |
| | | GGAGAAGATCGTGAAGGAGCGGAAGACCAGCAGCCGGATCCGGTTC |
| | | ATGCTGCAGGACGTTATCGACCTGCGGCTGTGCAACTGGGTGAGCCG |
| | | GAGGGCTGACCAGGGCCCCAAGACCATCGAGCAGATCCACAAGGAG |
| | | GCTAAGATCGAGGAGCAGGAAGAGCAGCGGAAGGTGCAGCAGCTG |
| | | ATGACCAAAGAGAAGCGGAGGCCTGGCGTGCAGCGAGTGGACGAG |
| | | GGCGGCTGGAACACCGTGCAGGGCGCCAAGAACAGCCGGGTGCTGG |
| | | ACCCCAGCAAGTTCCTGAAGATCACCAAGCCCACCATCGACGAGAA |
| | | GATCCAGCTGGTGCCCAAGGCCCAGCTCGGCAGCTGGGGCAAGGGC |
| | | AGCTCAGGT |
| 76 | Gld2 (aa) | FPNSILGRPPFTPNHQQHNNFFTLSPTVYSHQQLIDAQFNFQNADLS |
| | | RAVSLQQLTYGNVSPIQTSASPLFRGRKRLSDEKNLPLDGKRQRFHSPH |
| | | QEPTVVNQIVPLSGERRYSMPPLFHTHYVPDIVRCVPPFREIAFLEPREIT |
| | | LPEAKDKLSQQILELFETCQQQISDLKKKELCRTQLQREIQLLFPQSRLFL |
| | | VGSSLNGFGTRSSDGDLCLVVKEEPCFFQVNQKTEARHILTLVHKHFCT |
| | | RLSGYIERPQLIRAKVPIVKFRDKVSCVEFDLNVNNIVGIRNTFLLRTYA |
| | | YLENRVRPLVLVIKKWASHHQINDASRGTLSSYSLVLMVLHYLQTLPEP |
| | | ILPSLQKIYPESFSPAIQLHLVHQAPCNVPPYLSKNESNLGDLLLGFLKYY |
| | | ATEFDWNSQMISVREAKAIPRPDGIEWRNKYICVEEPFDGTNTARAVHE |
| | | KQKFDMIKDQFLKSWHRLKNKRDLNSILPVRAAVLKR |
| 77 | Gld2 (nt) | TTCCCTAACAGCATCCTGGGCAGACCTCCTTTCACCCCTAACCAC |
| | | CAGCAGCACAACAACTTCTTCACCCTGAGCCCTACCGTGTACAGCCA |
| | | TCAGCAGCTGATCGACGCCCAGTTCAACTTCCAGAACGCCGACCTGA |
| | | GCAGAGCCGTGAGCCTGCAGCAACTCACCTACGGCAACGTTAGCCC |
| | | AATCCAGACCAGCGCCAGCCCTCTGTTCAGAGGCAGAAAGAGACTG |
| | | AGCGACGAGAAGAACCTGCCTCTGGACGGCAAGAGACAGAGATTCC |
| | | ACAGCCCTCACCAGGAGCCAACAGTGGTGAACCAGATCGTGCCTCT |
| | | GAGCGGCGAGAGAAGATACAGCATGCCTCCACTGTTCCACACCCAC |
| | | TACGTGCCTGACATCGTGAGGTGCGTGCCACCTTTCAGAGAGATCGC |
| | | CTTCCTGGAGCCTCGCGAGATTACCCTGCCTGAGGCCAAGGACAAGC |
| | | TGAGCCAGCAGATTCTGGAACTGTTCGAGACTTGCCAACAACAAATT |
| | | AGCGACCTGAAGAAGAAGGAGCTGTGCAGAACCCAGCTGCAGCGCG |
| | | AGATCCAGCTGCTGTTCCCTCAGAGCAGACTGTTCCTGGTGGGCAGC |
| | | AGCCTGAACGGCTTCGGCACCAGAAGCAGCGACGGCGACCTGTGCC |
| | | TGGTGGTGAAGGAGGAGCCTTGCTTCTTCCAAGTAAATCAGAAGAC |
| | | CGAGGCCAGACACATCCTGACCCTGGTGCACAAGCACTTCTGCACCA |
| | | GACTGTCCGGCTACATCGAGCGCCCTCAGCTTATAAGAGCCAAGGTG |
| | | CCTATCGTGAAGTTCAGAGACAAGGTGAGCTGCGTGGAGTTCGATCT |
| | | GAACGTGAACAACATCGTGGGCATCAGAAACACCTTCCTGCTGAGA |
| | | ACCTACGCCTACCTGGAGAACAGAGTCAGACCTCTGGTGCTGGTGAT |
| | | CAAGAAGTGGGCTTCTCATCACCAGATCAACGACGCCTCCCGCGGTA |
| | | CTCTGTCTAGCTACAGCCTCGTACTGATGGTGCTGCACTACCTGCAG |
| | | ACTCTCCCTGAACCTATCCTGCCTAGTCTGCAGAAGATCTACCCTGA |
| | | GAGCTTCAGCCCTGCCATCCAATTGCATCTCGTTCACCAGGCCCCTT |
| | | GCAACGTTCCTCCTTATTTGAGCAAGAACGAGAGCAACCTGGGAGA |
| | | TCTCCTGCTGGGCTTCCTGAAGTACTACGCCACCGAATTCGATTGGA |
| | | ACAGCCAGATGATCAGCGTGAGAGAAGCAAAGGCCATCCCTAGACC |
| | | TGACGGCATCGAGTGGAGAAACAAGTACATCTGTGTGGAGGAACCT |
| | | TTCGACGGCACCAACACCGCCCGCGCAGTACACGAGAAGCAGAAGT |
| | | TCGACATGATCAAGGACCAGTTCCTCAAGAGCTGGCACAGACTGAA |
| | | GAACAAGAGAGACTTGAATAGCATCTTGCCGGTGAGAGCCGCCGTG |
| | | CTGAAGAGA |
| 78 | MBP-Gld2 (aa) | MASNFTQFVLVDNGGTGDVTVAPSNFANGIAEWISSNSRSQAYKV |
| | | TCSVRQSSAQNRKYTIKVEVPKGAWRSYLNMELTIPIFATNSDCELIVK |
| | | AMQGLLKDGNPIPSAIAANSGIYGGGGSFPNSILGRPPFTPNHQQHNNFF |
| | | TLSPTVYSHQQLIDAQFNFQNADLSRAVSLQQLTYGNVSPIQTSASPLFR |
| | | GRKRLSDEKNLPLDGKRQRFHSPHQEPTVVNQIVPLSGERRYSMPPLFH |
| | | THYVPDIVRCVPPFREIAFLEPREITLPEAKDKLSQQILELFETCQQQISDL |
| | | KKKELCRTQLQREIQLLFPQSRLFLVGSSLNGFGTRSSDGDLCLVVKEEP |
| | | CFFQVNQKTEARHILTLVHKHFCTRLSGYIERPQLIRAKVPIVKFRDKVS |
| | | CVEFDLNVNNIVGIRNTFLLRTYAYLENRVRPLVLVIKKWASHHQINDA |
| | | SRGTLSSYSLVLMVLHYLQTLPEPILPSLQKIYPESFSPAIQLHLVHQAPC |
| | | NVPPYLSKNESNLGDLLLGFLKYYATEFDWNSQMISVREAKAIPRPDGI |
| | | EWRNKYICVEEPFDGTNTARAVHEKQKFDMIKDQFLKSWHRLKNKRD |
| | | LNSILPVRAAVLKR |
| 79 | MBP-Gld2 (nt) | ATGGCCAGCAACTTCACCCAGTTCGTGCTGGTGGACAACGGCGG |
| | | TACCGGAGACGTGACCGTGGCCCCTTCTAACTTCGCCAACGGCATCG |
| | | CCGAGTGGATCAGCAGCAACAGCAGAAGCCAGGCCTACAAGGTGAC |
| | | CTGCAGCGTGAGACAGAGCAGCGCCCAGAACAGAAAGTACACCATC |
| | | AAGGTGGAGGTGCCTAAGGGCGCCTGGAGAAGCTACCTGAACATGG |
| | | AGCTGACCATCCCTATCTTCGCCACCAACAGCGACTGCGAGCTGATC |
| | | GTGAAGGCCATGCAGGGCCTGCTGAAGGACGGCAACCCTATCCCTA |
| | | GCGCCATCGCCGCCAATTCAGGCATCTACGGAGGCGGTGGAAGCTT |
| | | CCCTAACAGCATCCTGGGCAGACCTCCTTTCACCCCTAACCACCAGC |
| | | AGCACAACAACTTCTTCACCCTGAGCCCTACCGTGTACAGCCATCAG |
| | | CAGCTGATCGACGCCCAGTTCAACTTCCAGAACGCCGACCTGAGCA |
| | | GAGCCGTGAGCCTGCAGCAACTCACCTACGGCAACGTTAGCCCAAT |
| | | CCAGACCAGCGCCAGCCCTCTGTTCAGAGGCAGAAAGAGACTGAGC |
| | | GACGAGAAGAACCTGCCTCTGGACGGCAAGAGACAGAGATTCCACA |
| | | GCCCTCACCAGGAGCCAACAGTGGTGAACCAGATCGTGCCTCTGAG |
| | | CGGCGAGAGAAGATACAGCATGCCTCCACTGTTCCACACCCACTAC |
| | | GTGCCTGACATCGTGAGGTGCGTGCCACCTTTCAGAGAGATCGCCTT |
| | | CCTGGAGCCTCGCGAGATTACCCTGCCTGAGGCCAAGGACAAGCTG |
| | | AGCCAGCAGATTCTGGAACTGTTCGAGACTTGCCAACAACAAATTA |
| | | GCGACCTGAAGAAGAAGGAGCTGTGCAGAACCCAGCTGCAGCGCGA |
| | | GATCCAGCTGCTGTTCCCTCAGAGCAGACTGTTCCTGGTGGGCAGCA |
| | | GCCTGAACGGCTTCGGCACCAGAAGCAGCGACGGCGACCTGTGCCT |
| | | GGTGGTGAAGGAGGAGCCTTGCTTCTTCCAAGTAAATCAGAAGACC |
| | | GAGGCCAGACACATCCTGACCCTGGTGCACAAGCACTTCTGCACCA |
| | | GACTGTCCGGCTACATCGAGCGCCCTCAGCTTATAAGAGCCAAGGTG |
| | | CCTATCGTGAAGTTCAGAGACAAGGTGAGCTGCGTGGAGTTCGATCT |
| | | GAACGTGAACAACATCGTGGGCATCAGAAACACCTTCCTGCTGAGA |
| | | ACCTACGCCTACCTGGAGAACAGAGTCAGACCTCTGGTGCTGGTGAT |
| | | CAAGAAGTGGGCTTCTCATCACCAGATCAACGACGCCTCCCGCGGTA |
| | | CTCTGTCTAGCTACAGCCTCGTACTGATGGTGCTGCACTACCTGCAG |
| | | ACTCTCCCTGAACCTATCCTGCCTAGTCTGCAGAAGATCTACCCTGA |
| | | GAGCTTCAGCCCTGCCATCCAATTGCATCTCGTTCACCAGGCCCCTT |
| | | GCAACGTTCCTCCTTATTTGAGCAAGAACGAGAGCAACCTGGGAGA |
| | | TCTCCTGCTGGGCTTCCTGAAGTACTACGCCACCGAATTCGATTGGA |
| | | ACAGCCAGATGATCAGCGTGAGAGAAGCAAAGGCCATCCCTAGACC |
| | | TGACGGCATCGAGTGGAGAAACAAGTACATCTGTGTGGAGGAACCT |
| | | TTCGACGGCACCAACACCGCCCGCGCAGTACACGAGAAGCAGAAGT |
| | | TCGACATGATCAAGGACCAGTTCCTCAAGAGCTGGCACAGACTGAA |
| | | GAACAAGAGAGACTTGAATAGCATCTTGCCGGTGAGAGCCGCCGTG |
| | | CTGAAGAGA |
| 155 | eIF4G-mid2mut | DPTRLQGINCGPDFTPSFANLGRTTLSTRGPPRGGPGGELPRGPAGL |
| | (aa) | GPRRSQQGPRKEPRKIIATVLMTEDIKLNKAEKAWKPSSKRTAADKDR |
| | | GEEDADGSKTQDLFRRVRSILNKLTPQMFQQLMKQVTQLAIDTEERLK |
| | | GVIDLIFEKAISEPNFSVAYANMCRCLMALKVPTTEKPTVTVNFRKLLL |
| | | NRCQKEFEKDKDDDEVFEKKQKEMDEAATAEERGRLKEELEEARDIAR |
| | | RRSLGNIKAIGELFKLKMLTEAIMHDCVVKLLKNHDEESLECLCRLLTTI |
| | | GKDLDFEKAKPRMDQYFNQMEKIIKEKKTSSRIRFMLQDVLDLRGSNW |
| | | VPRRGDQGPKTIDQIHKEAEMEEHREHIKVQQLMAKGSDKRRGGPPGP |
| | | PISRGLPLVDDGGWNTVPISKGSRPIDTSRLTKITKPGSIDSNNQLFAPGG |
| | | RLSWGKGSSGGSGAKPSDAASEAARPATSTLNRFSALQQAV |
| 156 | eIF4G-mid2mut | GACCCTACCAGACTGCAGGGCATCAACTGCGGCCCTGACTTCAC |
| | (nt) | TCCTTCTTTCGCAAACCTGGGCAGAACCACCCTGAGCACCAGAGGCC |
| | | CTCCTAGAGGTGGTCCCGGCGGAGAACTCCCCAGGGGTCCTGCCGG |
| | | CCTGGGCCCTAGACGCTCCCAGCAAGGTCCTAGAAAGGAGCCAAGG |
| | | AAGATCATCGCCACCGTGCTGATGACCGAGGACATCAAGCTGAACA |
| | | AAGCTGAGAAGGCCTGGAAGCCTAGCAGCAAGAGAACCGCCGCCGA |
| | | CAAGGACAGAGGCGAGGAGGACGCCGACGGATCCAAGACCCAGGA |
| | | CCTGTTCAGAAGAGTGAGAAGCATCCTCAACAAGCTGACCCCTCAG |
| | | ATGTTCCAGCAGCTGATGAAGCAGGTGACGCAGCTCGCCATCGACA |
| | | CCGAGGAGAGACTGAAGGGCGTGATCGACCTGATCTTTGAGAAGGC |
| | | TATCTCAGAGCCTAACTTCAGCGTGGCCTACGCCAACATGTGCCGTT |
| | | GCCTGATGGCATTGAAGGTGCCAACCACCGAGAAGCCTACTGTGAC |
| | | CGTCAATTTCCGTAAACTGCTGCTGAACCGGTGCCAGAAAGAGTTCG |
| | | AGAAGGATAAGGACGACGACGAGGTCTTCGAGAAGAAACAGAAAG |
| | | AAATGGACGAGGCCGCCACCGCAGAGGAAAGGGGCCGATTAAAGG |
| | | AGGAGCTGGAGGAGGCCAGAGACATCGCCAGACGGCGTTCTCTGGG |
| | | CAACATCAAGGCGATAGGTGAGCTGTTCAAGCTAAAGATGCTCACC |
| | | GAGGCCATAATGCACGACTGCGTGGTGAAGCTACTGAAGAACCACG |
| | | ACGAAGAAAGCCTGGAGTGCCTGTGCAGACTGCTGACCACCATCGG |
| | | CAAGGACCTGGACTTCGAGAAGGCAAAGCCTCGAATGGACCAGTAC |
| | | TTCAACCAGATGGAGAAGATTATCAAGGAGAAGAAGACCAGCAGCA |
| | | GAATCAGATTCATGCTGCAGGACGTACTGGACCTGCGCGGAAGCAA |
| | | CTGGGTGCCAAGGAGAGGGGACCAAGGACCAAAGACCATCGACCA |
| | | GATCCACAAGGAAGCGGAGATGGAGGAGCACAGAGAGCACATAAA |
| | | GGTGCAGCAGCTTATGGCCAAGGGCAGCGACAAGCGAAGAGGCGGC |
| | | CCGCCCGGACCTCCTATCAGCAGAGGCCTTCCTCTGGTAGACGACGG |
| | | CGGCTGGAACACCGTGCCTATCTCTAAGGGCTCCAGACCTATCGACA |
| | | CTTCCCGTCTTACCAAGATCACCAAGCCAGGATCTATTGACAGCAAC |
| | | AACCAGCTGTTCGCCCCAGGAGGAAGACTTAGCTGGGGCAAGGGAA |
| | | GTTCCGGCGGATCCGGCGCCAAGCCTTCCGACGCCGCCAGCGAGGC |
| | | TGCCAGACCTGCCACCAGCACCTTGAACCGCTTTTCCGCTCTGCAGC |
| | | AAGCTGTG |

### Tether Molecule

Disclosed herein, inter cilia, is a system or LNP comprising a polynucleotide, e.g., a second polynucleotide, e.g., mRNA, encoding an RNA-binding protein, e.g., a tether molecule. In some embodiments, the second polynucleotide encodes a effector molecule which further comprises a tether molecule.

In some embodiments, a system or LNP disclosed herein comprises a first polynucleotide comprising a binding element. In some embodiments, the tether molecule, e.g., effector molecule further comprising a tether molecule, binds to a binding element in the first polynucleotide. In some embodiments, the tether molecule, e.g., effector molecule further comprising a tether molecule, binds to a sequence of the binding element or to a structure comprising the sequence of the binding element. In some embodiments, a tether molecule comprises an RNA-binding protein or a variant or a fragment thereof. Exemplary RNA-binding proteins are provided in Tables 1 and 4.

In some embodiments, the tether molecule comprises a tether molecule provided in Table 1, e.g., MBP, PCP, Lambda N, U1A or PUF, 15.5 kd, LARP7 or a variant or fragment thereof. In some embodiments, the tether molecule is MBP. In some embodiments, the tether molecule is PCP. In some embodiments, the tether molecule is Lambda N. In some embodiments, the tether molecule is U1A. In some embodiments, the tether molecule is PUF. In some embodiments, the tether molecule is 15.5 kd. In some embodiments, the tether molecule is LARP7.

In some embodiments, when the tether molecule is MBP (e.g., wildtype MBP, a variant or fragment thereof) the binding element is MS2 (e.g., wildtype MS2, or a variant or fragment thereof).

In some embodiments, when the tether molecule is PCP (e.g., wildtype PCP, or a variant or fragment thereof) the binding element is PP7 (e.g., wildtype PP7, or a variant or fragment thereof).

In some embodiments, when the tether molecule is Lambda N (e.g., wildtype Lambda N, or a variant or fragment thereof) the binding element is BoxB (e.g., wildtype BoxB, or a variant or fragment thereof).

In some embodiments, when the tether molecule is U1A (e.g., wildtype U1A, or a variant or fragment thereof) the binding element is U1A hairpin (e.g., wildtype U1A hairpin, or a variant or fragment thereof).

In some embodiments, when the tether molecule is 15.5 kd (e.g., wildtype 15.5 kd, or a variant or fragment thereof) the binding element is a kink-turn forming sequence (e.g., wildtype U1A hairpin, or a variant or fragment thereof).

In some embodiments, when the tether molecule is PUF (e.g., wildtype PUF, or a variant or fragment thereof) the binding element is PRE (e.g., wildtype PRE, or a variant or fragment thereof).

In some embodiments, when the tether molecule is LARP7 (e.g., wildtype LARP7, or a variant or fragment thereof) the binding element is 7SK (e.g., wildtype 7SK, or a variant or fragment thereof).

Additional exemplary RNA-binding proteins or RNA-binding domains which can be used as tether molecules are disclosed in Corley et al, Molecular Cell 78:1 pp. 9-29, the entire contents of which are hereby incorporated by reference. For example, Table 3 provides additional exemplary RNA-binding proteins or domains which can be used as tether molecules. In an embodiment, a tether molecule disclosed herein comprises a domain (or a variant, or a fragment thereof) or a protein (or a variant or a fragment thereof) listed in Table 3.

TABLE 3
Exemplary RNA-binding proteins and domains.

| Domain name | Protein family containing domain |
|---|---|
| Cold shock domain | Cold shock proteins, Y-box |
| | proteins |
| Double stranded RNA | RNAses, ADARs, DICER |
| binding domain | |
| Helicase | DExH/D-box, Ski2-like, RIGI- |
| | like, NS3, UPF1-like RNA |
| | binding helicases |
| Intrinsically disordered | Most RBPs |
| regions (IDR) | |
| K homology | hnRNPs, translation regulation |
| | proteins, |
| La motif (LAM) | La proteins, La-related proteins |
| | (LARPs) |
| Piwi-Argonaute-Zwille | Argonaute proteins, Dicer |
| (PAZ) | |
| P-element Induced Wimpy | Argonaute proteins, |
| Testis (PIWI) | |
| Pentatricopeptide repeat (PPR) | RNA editing proteins |
| Pseudouridine synthase and | RNA modifying enzymes, |
| archaeosine | metabolic benzymes |
| transglycoslyase (PUA) | |
| Pumillo-like repeat (PUM) | PUF proteins |
| Ribosomal S1-like | Ribosomal proteins, Translation |
| | initiation factors, RNase II, |
| | PNPase |
| RNA recognition motif (RRM) | hnRNPs, splicing factors, |
| Sm and Like-Sm (Sm/Lsm) | Ul spliceosomal proteins, Hfq |
| thiouridine synthases, RNA | tRNA modifying enzyme |
| methylases and pseudouridine | |
| synthases (THUMP) | |
| YT521-B homology (YTH) | YTH family m6A readers |
| Zn finger | Transcription factors, METTL |
| | enzymes, |

In some embodiments, the tether molecule comprises MBP. In some embodiments, the tether molecule comprises an amino acid sequence provided in Table 2 or a sequence with at least 80%, 85%, 90%, 95%, 96%, 97%, 98%, 99% or 100% identity thereof. In some embodiments, the tether molecule comprises the amino acid sequence of SEQ ID NO: 6, or an amino acid sequence with at least 80%, 85%, 90%, 95%, 96%, 97%, 98%, 99% or 100% identity thereof.

In some embodiments, the tether molecule comprises is encoded by a nucleotide sequence provided in Table 2 or a sequence with at least 80%, 85%, 90%, 95%, 96%, 97%, 98%, 99% or 100% identity thereof. In some embodiments, the tether molecule comprises is encoded by the nucleotide sequence of SEQ ID NO: 7, or a sequence with at least 80%, 85%, 90%, 95%, 96%, 97%, 98%, 99% or 100% identity thereof.

### Effector Molecule

Disclosed herein, inter alfa, is a system or LNP comprising a polynucleotide, e.g., a second polynucleotide, e.g., mRNA, encoding an effector molecule. In some embodiments, the effector molecule is chosen from a factor provided in Table 4, e.g., a translation factor, a splicing factor, an RNA stabilizing factor, an RNA editing factor, an RNA-binding factor (e.g., PABP that binds the polyA tail of an mRNA), an RNA localizing factor, or an RNA modulating factor (such as Gld2, TENT 4A and TENT4B which are known to add more As and or A/G nucleotides to the polyA tail of an mRNA) or a combination thereof.

In some embodiments, the effector molecule is a translation factor, e.g., a translation factor provided in Table 4, e.g., eIF4G; Poly A binding protein (PABP); eIF3d or a component thereof; Daz1, or a fragment, or variant or combination thereof. Additional examplary translation factors are provided in Pelletier and Soneneberg. Annu. Rev. Biochem. 2019. 88:307-35, the entire contents of which are hereby incorporated by reference

In some embodiments, the effector molecule is a splicing factor, e.g., a splicing factor provided in Table 4, e.g., Rups1, Magoh, Y14, or a fragment or variant, or combination thereof. Additional splicing factors are provided in Nott et al, (2004) Genes & Dev, 2004. 18, 210-222, the entire contents of which are hereby incorporated by reference.

In some embodiments, the effector molecule is an RNA stabilizing factor, e.g., a stabilizing factor provided in Table 4, e.g., HuR or a fragment, or variant thereof. Exemplary RNA stabilizing factors are provided in Goldberg et al., (2002) J Biol Chem. 2002 Apr. 19; 277(16):13635-40 , the entire contents of which are hereby incorporated by reference.

In some embodiments, the effector molecule is an RNA editing factor. Exemplary RNA editing factors are provided in Kim D. et al (2019), Ann Rev Biochem, 88, 191-200, the entire contents of which are hereby incorporated by reference. In some embodiments, the effector molecule is an RNA binding factor.

Exemplary RNA binding factors are provided in Singh G. et al (2015) Annu Rev. Biochem; 84: 325-354, the entire contents of which are hereby incorporated by reference.

In some embodiments, the effector molecule is an RNA localizing factor. Exemplary RNA localizing factors are provided in Blower M.D. (2013) Int Rev Cell Mol Biol.; 302: 1-39, the entire contents of which are hereby incorporated by reference.

In some embodiments, the effector molecule is an RNA modulating factor. Exemplary RNA factors are provided in Philos Trans R Soc Lond B Biol Sci. 2018 Dec. 19; 373(1762), the entire contents of which are hereby incorporated by reference.

TABLE 4
Exemplary effector molecules

| Effector molecule | Examples |
|---|---|
| Translation factor | eIF4G; Poly A binding protein (PABP); eIF3d; Dazl |
| Splicing factor | Rnps1, Magoh, Y14 |
| RNA stabilizing | HuR |
| factor | Exemplary RNA stabilizing factors provided in |
| | Goldberg et al., (2002) J Biol Chem. 2002 Apr. |
| | 19; 277(16): 13635-40 |
| RNA editing | Exemplary RNA editing factors are provided in Kim |
| factor | D. et al (2019), Ann Rev Biochem, 88, 191-200 |
| RNA binding | Exemplary RNA binding factors provided in Singh |
| factor | G. et al (2015) Annu Rev. Biochem; 84: 325-354 |
| RNA localizing | Blower M. D. (2013) Int Rev Cell Mol Biol.; 302: 1- |
| factor | 39 |
| RNA modulating | Philos Trans R Soc Lond B Biol Sci. 2018 Dec. 19; |
| factor | 373(1762) |

In some embodiments, the effector molecule binds directly to the binding element. The effector molecule may have a specific target sequence to which it can bind. Effector molecules include, but are not limited to eIF4G, eIF4d, PABPC, TENT4A, TENT4B, and Gld2.

In some embodiments, the effector molecule further comprises a polypeptide that binds to, e.g., recognizes, the binding element (a tether molecule).

In an embodiment, the effector molecule polypeptide comprising a tether molecule comprises a first domain which modulates a parameter of, e.g., level and/or activity of: an RNA (e.g., an mRNA); or a protein encoded by the RNA. In an embodiment, the parameter comprises one, two, three or all of: (1) mRNA level and/or activity and/or subcellular localization (e.g., half-life and/or expression); (2) protein level and/or activity (e.g., half-life and/or expression); (3) protein translation rate or (4) protein localization, e.g., location. In an embodiment the effector molecule polypeptide comprising a tether molecule comprises a second domain which binds to, e.g., recognizes, the binding element (a tether molecule).

In an embodiment the effector molecule comprising the tether molecule comprises a polypeptide comprising the first domain and the second domain. In an embodiment, the first and second domains are operatively linked.

In an embodiment, in the second polynucleotide encoding the effector molecule which further comprises a tether molecule, the nucleotide sequence encoding the effector molecule is upstream of the nucleotide sequence encoding the tether molecule. In an embodiment, the nucleotide sequence encoding the effector molecule is downstream of the nucleotide sequence encoding the tether molecule. In an embodiment, the nucleotide sequence encoding the effector molecule is separated from the nucleotide sequence encoding the tether molecule by a protease cleavage site (e.g., a P2A, T2A, E2A, or TPE (P2A-T2A-E2A) site) or an internal ribosomal entry site.

In an embodiment, in the second polynucleotide encoding the effector molecule which further comprises a tether molecule, the nucleotide sequence encoding the effector molecule is adjacent to the nucleotide sequence encoding the tether molecule.

In some embodiments, the effector molecule is a translation factor which modulates, e.g., facilitates, ribosome binding, e.g., recruitment, pre-initiation complex formation, or RNA unwinding. In some embodiments, the effector molecule comprises eIF4G, e.g., wildtype eIF4G, a variant of eIF4G, or a fragment thereof.

In some embodiments, the effector molecule comprises wildtype eIF4G. In some embodiments, wildtype eIF4G comprises a sequence of about 1600 amino acids.

In some embodiments, the effector molecule comprises a fragment of eIF4G, e.g., as disclosed herein. In some embodiments, the eIF4G fragment retains ribosome binding, e.g., recruitment.

In some embodiments, the eIF4G fragment is about 1,500-200 amino acids, about 1,400-300 amino acids, about 1,300-350 amino acids, about 1,200-400 amino acids, about 1,100-450 amino acids, about 1,000-500 amino acids, about 900-550 amino acids, about 800-600 amino acids, about 1,500-300 amino acids, 1,500-400 amino acids, 1,500-500 amino acids, about 1,500-600 amino acids, amino acids, about 1,500-700 amino acids, about 1,500-800 amino acids, about 1,500-900 amino acids, about 1,500-1000 amino acids, about 1,500-1,100 amino acids, about 1,500-1,200 amino acids, about 1,500-1,300 amino acids, about 1,500-1,400 amino acids, about 1,400-200 amino acids, about 1,300-200 amino acids, about 1,200-200 amino acids, about 1,100-200 amino acids, about 1,000-200 amino acids, about 900-200 amino acids, about 800-200 amino acids, about 700-200 amino acids, about 600-200 amino acids, or about 500-200 amino acids in length.

In some embodiments, the eIF4G fragment is about 500 amino acids in length. In some embodiments, the eIF4G fragment is about 600 amino acids in length. In some embodiments, the eIF4G fragment is about 700 amino acids in length. In some embodiments, the eIF4G fragment is about 800 amino acids in length. In some embodiments, the eIF4G fragment is about 900 amino acids in length. In some embodiments, the eIF4G fragment is about 1000 amino acids in length. In some embodiments, the eIF4G fragment is about 1100 amino acids in length. In some embodiments, the eIF4G fragment is about 1200 amino acids in length. In some embodiments, the eIF4G fragment is about 1300 amino acids in length. In some embodiments, the eIF4G fragment is about 1400 amino acids in length. In some embodiments, the eIF4G fragment is about 1500 amino acids in length.

In some embodiments, the effector molecule comprises a variant of eIF4G, e.g., as disclosed herein. In some embodiments, the eIF4G variant retains ribosome binding, e.g., recruitment. In some embodiments, the eIF4G variant comprises a mutation (e.g., substitution) in the eIF4G polypeptide sequence at any one, two, all or a combination of the following positions: amino acid 768, amino acid 771, or amino acid 776. In some embodiments, the eIF4G variant comprises a mutation, e.g., substitution, at position 768 of the eIF4G polypeptide sequence, e.g., a Leucine to Alanine substitution at position 768. In some embodiments, the eIF4G variant comprises a mutation, e.g., substitution, at position 771 of the eIF4G polypeptide sequence, e.g., a Leucine to Alanine substitution at position 771. In some embodiments, the eIF4G variant comprises a mutation, e.g., substitution, at position 776 of the eIF4G polypeptide sequence, e.g., a Phenylalanine to Alanine at position 776. In some embodiments, the eIF4G variant comprises a mutation, e.g., substitution, at position 768 of the eIF4G polypeptide sequence, e.g., an Alanine at position 768; and a mutation, e.g., substitution, at position 771 of the eIF4G polypeptide sequence, e.g., an Alanine at position 771. In some embodiments, the eIF4G variant comprises a mutation, e.g., substitution, at position 768 of the eIF4G polypeptide sequence, e.g., an Alanine at position 768; and a mutation, e.g., substitution, at position 776 of the eIF4G polypeptide sequence, e.g., an Alanine at position 776. In some embodiments, the eIF4G variant comprises a mutation, e.g., substitution, at position 771 of the eIF4G polypeptide sequence, e.g., an Alanine at position 771; and a mutation, e.g., substitution, at position 776 of the eIF4G polypeptide sequence, e.g., an Alanine at position 776. In some embodiments, the eIF4G variant comprises a mutation, e.g., substitution, at position 771 of the eIF4G polypeptide sequence, e.g., an Alanine at position 771; a mutation, e.g., substitution, at position 771 of the eIF4G polypeptide sequence, e.g., an Alanine at position 771; and a mutation, e.g., substitution, at position 776 of the eIF4G polypeptide sequence, e.g., an Alanine at position 776.

In some embodiments, the effector molecule is a part of the eIF3 complex, e.g., which can recruit the ribosome. In some embodiments, the eIF3 complex comprises eIF3d, eIF3c, eIF3e, or eIF3i, or a fragment thereof, or any combination thereof.

In some embodiments, the effector molecule, e.g., eIF4G, PABP, TENT4A, TENT4B, or Gld2 comprises an amino acid sequence provided in Table 2, or a sequence with at least 80%, 85%, 90%, 95%, 96%, 97%, 98%, 99% or 100% identity thereof.

In some embodiments, the effector molecule, e.g., eIF4G, comprises SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO:22, SEQ ID NO: 24, SEQ ID NO: 26 SEQ ID NO: 28, SEQ ID NO: 30, SEQ ID NO: 32, SEQ ID NO: 34, SEQ ID NO: 36, SEQ ID NO: 40, SEQ ID NO: 42, SEQ ID NO: 44, SEQ ID NO: 46, SEQ ID NO: 48, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, or SEQ ID NO: 155, or a sequence with at least 80%, 85%, 90%, 95%, 96%, 97%, 98%, 99% or 100% identity thereof.

In some embodiments, the effector molecule, e.g., PABP, comprises SEQ ID NO: 48, or SEQ ID NO: 50, or a sequence with at least 80%, 85%, 90%, 95%, 96%, 97%, 98%, 99% or 100% identity thereof.

In some embodiments, the effector molecule, e.g., TENT4A, comprises SEQ ID NO: 52, or SEQ ID NO: 54, or a sequence with at least 80%, 85%, 90%, 95%, 96%, 97%, 98%, 99% or 100% identity thereof.

In some embodiments, the effector molecule, e.g., TENT4B, comprises SEQ ID NO: 56, or SEQ ID NO: 58, or a sequence with at least 80%, 85%, 90%, 95%, 96%, 97%, 98%, 99% or 100% identity thereof.

In some embodiments, the effector molecule, e.g., Gld2, comprises SEQ ID NO:

76, or SEQ ID NO: 78, or a sequence with at least 80%, 85%, 90%, 95%, 96%, 97%, 98%, 99% or 100% identity thereof.

In some embodiments, the effector molecule e.g., eIF4G, PABP, TENT4A, TENT4B, or Gld2 is encoded by a nucleotide sequence provided in Table 2, or a sequence with at least 80%, 85%, 90%, 95%, 96%, 97%, 98%, 99% or 100% identity thereof.

In some embodiments, the effector molecule e.g., eIF4G, is encoded by the nucleotide sequence of SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO:

35, SEQ ID NO: 37, SEQ ID NO: 41, SEQ ID NO: 43, SEQ ID NO: 45, SEQ ID NO: 47, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 156 or a sequence with at least 80%, 85%, 90%, 95%, 96%, 97%, 98%, 99% or 100% identity thereof.

In some embodiments, the effector molecule, e.g., PABP, is encoded by the nucleotide sequence of SEQ ID NO: 49, or SEQ ID NO: 51, or a sequence with at least 80%, 85%, 90%, 95%, 96%, 97%, 98%, 99% or 100% identity thereof.

In some embodiments, the effector molecule, e.g., TENT4A, comprises SEQ ID NO: 53, or SEQ ID NO: 55, or a sequence with at least 80%, 85%, 90%, 95%, 96%, 97%, 98%, 99% or 100% identity thereof.

In some embodiments, the effector molecule, e.g., TENT4B, comprises SEQ ID NO: 57, or SEQ ID NO: 59, or a sequence with at least 80%, 85%, 90%, 95%, 96%, 97%, 98%, 99% or 100% identity thereof.

In some embodiments, the effector molecule, e.g., Gld2, comprises SEQ ID NO: 77, or SEQ ID NO: 79, or a sequence with at least 80%, 85%, 90%, 95%, 96%, 97%, 98%, 99% or 100% identity thereof.

### Lipid Content of LNPs

As set forth above, with respect to lipids, LNPs disclosed herein comprise an (i) ionizable lipid; (ii) sterol or other structural lipid; (iii) a non-cationic helper lipid or phospholipid; and, optionally a (iv) PEG lipid. These categories of lipids are set forth in more detail below. In some embodiments, nucleic acids of the invention are formulated as lipid nanoparticle (LNP) compositions.

Lipid nanoparticles typically comprise amino lipid, phospholipid, structural lipid and PEG lipid components along with the nucleic acid cargo of interest. The lipid nanoparticles of the invention can be generated using components, compositions, and methods as are generally known in the art, see for example PCT/US2016/052352; PCT/U52016/068300; PCT/U52017/037551; PCT/U52015/027400; PCT/US2016/047406; PCT/US2016000129; PCT/U52016/014280; PCT/U52016/014280; PCT/US2017/038426; PCT/US2014/027077; PCT/U52014/055394; PCT/U52016/52117; PCT/U52012/069610; PCT/US2017/027492; PCT/U52016/059575; PCT/US2016/069491; PCT/US2016/069493; and PCT/US2014/66242, all of which are incorporated by reference herein in their entirety.

In some embodiments, the lipid nanoparticle comprises a molar ratio of 20-60% amino lipid relative to the other lipid components. For example, the lipid nanoparticle may comprise a molar ratio of 20-50%, 20-40%, 20-30%, 30-60%, 30-50%, 30-40%, 40-60%, 40-50%, or 50-60% amino lipid. In some embodiments, the lipid nanoparticle comprises a molar ratio of 20%, 30%, 40%, 50, or 60% amino lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises a molar ratio of 20-60% amino lipid, 5-25% phospholipid, 25-55% structural lipid, and 0.5-15% PEG lipid.

In some embodiments, the lipid nanoparticle comprises a molar ratio of 20-60% amino lipid, 5-30% phospholipid, 10-55% structural lipid, and 0.5-15% PEG lipid.

Amino lipids In some aspects, the amino lipids of the present disclosure may be one or more of compounds of Formula (I):

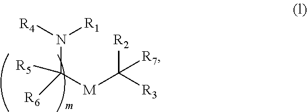

or their N-oxides, or salts or isomers thereof, wherein:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of hydrogen, a C₃₋₆ carbocycle, —(CH₂)ₙQ, —(CH₂)ₙCHQR,

—CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CX₂, —CN, -N(R)₂, —C(O)N(R)₂, —N(R)C(O)R, —N(R)S(O)₂R, —N(R)C(O)N(R)₂, —N(R)C(S)N(R)₂, —N(R)R₈,
—N(R)S(O)₂R₈, —O(CH₂)ₙOR, —N(R)C(═NR₉)N(R)₂, —N(R)C(=CHR₉)N(R)₂, —OC(O)N(R)₂,

—N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;

each Rs is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —OC(O)-M″-C(O)O—, —C(O)N(R′)—,

—N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In certain embodiments, a subset of compounds of Formula (I) includes those of Formula (IA):

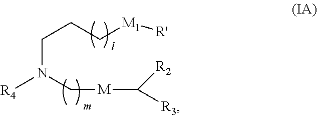

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″—C(O)O—, —C(O)N(R′)—, —P(O)(OR′)O—, —S—S—, an aryl group, and a heteroaryl group,; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB):

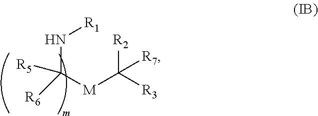

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″—C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (II):

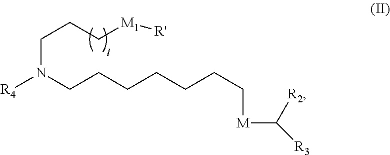

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC (O)N(R)₂, —N(R)C(O)R, —N(R) S (O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″—C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In one embodiment, the compounds of Formula (I) are of Formula (IIa),

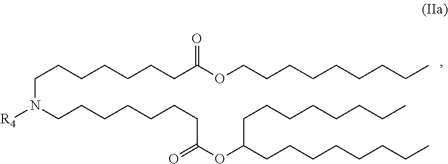

or their N-oxides, or salts or isomers thereof, wherein R₄ is as described herein.

In another embodiment, the compounds of Formula (I) are of Formula (IIb),

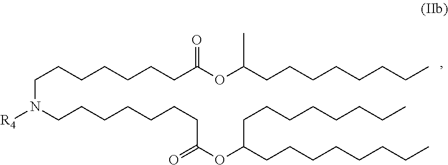

or their N-oxides, or salts or isomers thereof, wherein R₄ is as described herein.

In another embodiment, the compounds of Formula (I) are of Formula (IIc) or

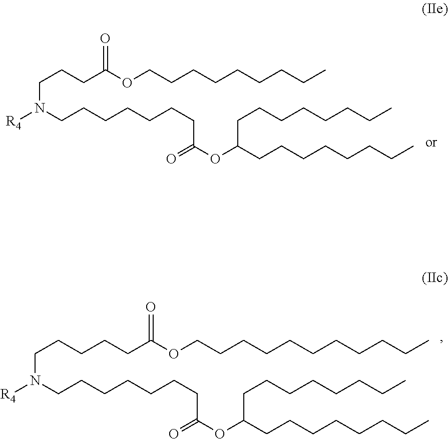

or their N-oxides, or salts or isomers thereof, wherein R₄ is as described herein.

In another embodiment, the compounds of Formula (I) are of Formula (ITO:

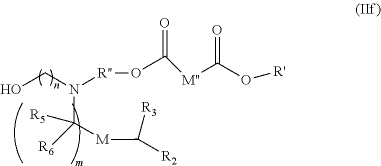

or their N-oxides, or salts or isomers thereof,

wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (I) are of Formula (IId),

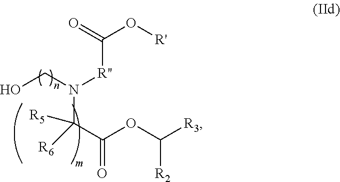

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In a further embodiment, the compounds of Formula (I) are of Formula (IIg),

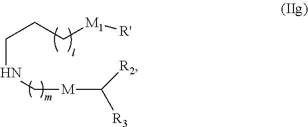

or their N-oxides, or salts or isomers thereof, wherein 1 is selected from 1, 2, 3, 4, and 5; m is selected from 5, 6, 7, 8, and 9; Mi is a bond or M′; M and M′ are independently selected from —C(O)O—, —OC(O)—, —OC(O)-M″—C(O)O—, —C(O)N(R′)—, —P(O)(OR′)O—,—S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

In some embodiments, the amino lipid is

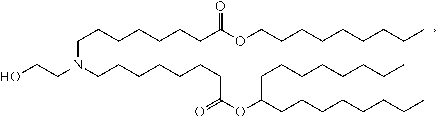 [SMILES: CCCCCCCCCOC(=O)CCCCCCCN(CCO)CCCCCCCC(=O)OC(CCCCCCCC)CCCCCCCC]

or a salt thereof.

In some embodiments, the amino lipid is

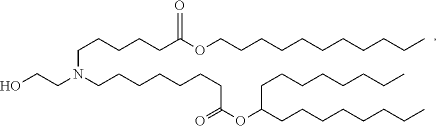 [SMILES: CCCCCCCCCCCOC(=O)CCCCCN(CCO)CCCCCCCC(=O)OC(CCCCCCCC)CCCCCCCC]

or a salt thereof.

The central amine moiety of a lipid according to Formula (I), (IA), (IB), (II), (IIa), (IIb), (IIc), (IId), (IIe), (IIf), or (IIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some aspects, the amino lipids of the present disclosure may be one or more of compounds of formula (III),

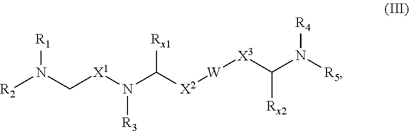

or salts or isomers thereof, wherein

W is

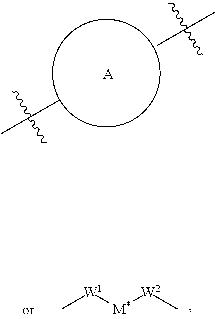

ring A is

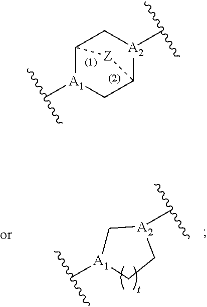

t is 1 or 2;

A₁ and A₂ are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

R_{X1} and R_{X2} are each independently H or C₁₋₃ alkyl;

each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R₆)—;

each R₆ is independently selected from the group consisting of H and C₁₋₅ alkyl;

X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and —R*MR′; and

n is an integer from 1-6;

wherein when ring A is

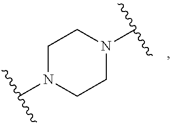 [SMILES: *N1CCN(*)CC1]

then

i) at least one of X¹, X², and X³ is not —CH₂—; and/or

ii) at least one of R₁, R₂, R₃, R₄, and R₅ is —R″MR′.

In some embodiments, the compound is of any of formulae (IIIa1)-(IIIa8):

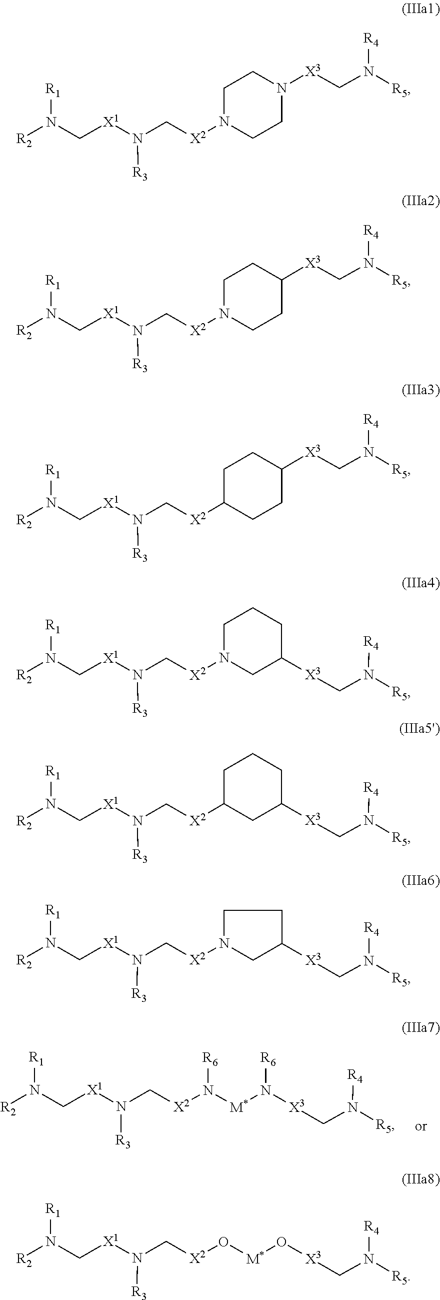

In some embodiments, the amino lipid is

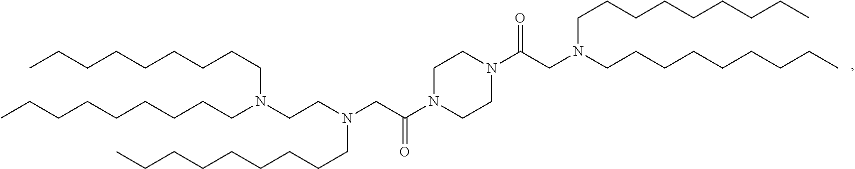 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (III), (IIIa1), (IIIa2), (IIIa3), (IIIa4), (IIIa5), (IIIa6), (IIIa7), or (IIIa8) may be protonated at a physiological pH. Thus, a lipid may have a positive or partial positive charge at physiological pH. Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-Di stearoyl-sn-glycero-3-phosphoethanolamine (D SPE), 1,2-di oleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

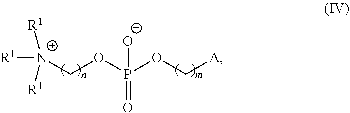

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three le are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

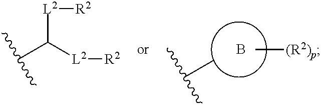

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), —OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

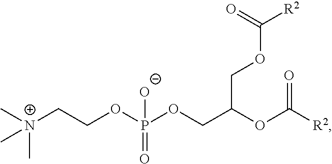

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530, or in International Application PCT/US2018/037922 filed on 15 Jun. 2018, the entire contents of each of hich is hereby incorporated by reference in its entirety.

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

### Polyethylene Glycol (PEG)-Lipids

The lipid composition of a pharmaceutical composition disclosed herein can comprise one or more polyethylene glycol (PEG) lipids.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

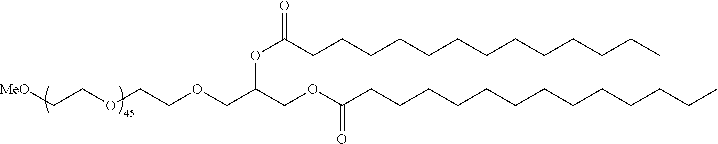

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

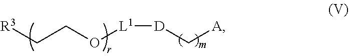

or salts thereof, wherein:

R³ is —OR^{O};

R^{O} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), —C(O)N(R^{N}), NR^{N}C(O), C(O)O, OC(O), OC(O)O, OC(O)N(R^{N}), NR^{N}C(O)O, or —NR^{N}C(O)N(R^{N});

D is a moiety obtained by click chemistry or a moiety cleavable under physiological conditions;

m is 0, 1, 2, 3, 4, 5, 6, 7, 8, 9, or 10;

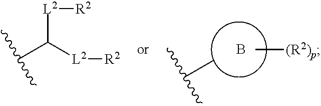

A is of the formula:

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), —OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), —NR^{N}C(S), NR^{N}C(S)N(R^{N}), S(O) , OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Fomula (V) is a PEG-OH lipid (i.e., R³ is —OR^{O}, and R^{O} is hydrogen). In certain embodiments, the compound of Formula (V) is of Formula (V-OH):

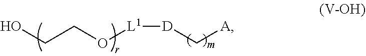

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI-A):

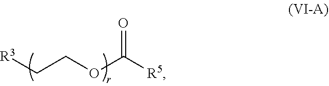

or a salts thereof, wherein:

R³ is—OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, —N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), S^{N}C(^{—}N^{N}), NR^{N}C(═NR^{N})N(R^{N}), C(S), C(S)N(R^{N}), NR^{N}C(S), NR^{N}C(S)N(R^{N}), S(O), OS(O), —S(O)O, OS(O)O, OS(O)₂, S(O)₂O, OS(O)₂O, N(R^{N})S(O), S(O)N(R^{N}), —N(R^{N})S(O)N(R^{N}), OS(O)N(R^{N}), N(R^{N})S(O)O, S(O)₂, N(R^{N})S (O)₂, S(O)₂N(R^{N}), —N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (VI) is of Formula (VI-OH):

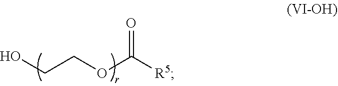

also referred to as (VI-B),

or a salt thereof. In some embodiments, r is 40-50.

In yet other embodiments the compound of Formula (VI-C) is:

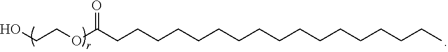

or a salt thereof.

In one embodiment, the compound of Formula (VI-D) is

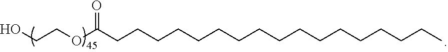

In some aspects, the lipid composition of the pharmaceutical compositions disclosed herein does not comprise a PEG-lipid.

In some embodiments, the PEG-lipids may be one or more of the PEG lipids described in U.S. application Ser. No. 15/674,872.

In some embodiments, a LNP of the invention comprises an amino lipid of any of Formula I, II or III, a phospholipid comprising DSPC, a structural lipid, and a PEG lipid comprising PEG-DMG.

In some embodiments, a LNP of the invention comprises an amino lipid of any of Formula I, II or III, a phospholipid comprising DSPC, a structural lipid, and a PEG lipid comprising a compound having Formula VI.

In some embodiments, a LNP of the invention comprises an amino lipid of Formula I, II or III, a phospholipid comprising a compound having Formula IV, a structural lipid, and the PEG lipid comprising a compound having Formula V or VI.

In some embodiments, a LNP of the invention comprises an amino lipid of Formula I, II or III, a phospholipid comprising a compound having Formula IV, a structural lipid, and the PEG lipid comprising a compound having Formula V or VI.

In some embodiments, a LNP of the invention comprises an amino lipid of Formula I, II or III, a phospholipid having Formula IV, a structural lipid, and a PEG lipid comprising a compound having Formula VI.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1, 4:1, or 5:1.

In some embodiments, a LNP of the invention comprises a wt/wt ratio of the amino lipid component to the RNA of from about 10:1 to about 100:1.

In some embodiments, a LNP of the invention comprises a wt/wt ratio of the amino lipid component to the RNA of about 20:1.

In some embodiments, a LNP of the invention comprises a wt/wt ratio of the amino lipid component to the RNA of about 10:1.

In some embodiments, a LNP of the invention has a mean diameter from about 30 nm to about 150 nm.

In some embodiments, a LNP of the invention has a mean diameter from about 60 nm to about 120 nm.

### Exemplary Additional LNP Components

### Surfactants

In certain embodiments, the lipid nanoparticles of the disclosure optionally includes one or more surfactants.

In certain embodiments, the surfactant is an amphiphilic polymer. As used herein, an amphiphilic “polymer” is an amphiphilic compound that comprises an oligomer or a polymer. For example, an amphiphilic polymer can comprise an oligomer fragment, such as two or more PEG monomer units. For example, an amphiphilic polymer described herein can be PS 20.

For example, the amphiphilic polymer is a block copolymer.

For example, the amphiphilic polymer is a lyoprotectant.

For example, amphiphilic polymer has a critical micelle concentration (CMC) of less than 2×10-4 M in water at about 30° C. and atmospheric pressure.

For example, amphiphilic polymer has a critical micelle concentration (CMC) ranging between about 0.1×10-4 M and about 1.3×10-4 M in water at about 30° C. and atmospheric pressure.

For example, the concentration of the amphiphilic polymer ranges between about its CMC and about 30 times of CMC (e.g., up to about 25 times, about 20 times, about 15 times, about 10 times, about 5 times, or about 3 times of its CMC) in the formulation, e.g., prior to freezing or lyophilization.

For example, the amphiphilic polymer is selected from poloxamers (Pluronic®), poloxamines (Tetronic®), polyoxyethylene glycol sorbitan alkyl esters (polysorbates) and polyvinyl pyrrolidones (PVPs).

For example, the amphiphilic polymer is a poloxamer. For example, the amphiphilic polymer is of the following structure:

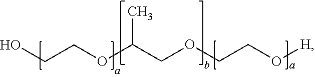

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

For example, the amphiphilic polymer is P124, P188, P237, P338, or P407.

For example, the amphiphilic polymer is P188 (e.g., Poloxamer 188, CAS Number 9003-11-6, also known as Kolliphor P188).

For example, the amphiphilic polymer is a poloxamine, e.g., tetronic 304 or tetronic 904.

For example, the amphiphilic polymer is a polyvinylpyrrolidone (PVP), such as PVP with molecular weight of 3 kDa, 10 kDa, or 29 kDa.

For example, the amphiphilic polymer is a polysorbate, such as PS 20.

In certain embodiments, the surfactant is a non-ionic surfactant.

In some embodiments, the lipid nanoparticle comprises a surfactant. In some embodiments, the surfactant is an amphiphilic polymer. In some embodiments, the surfactant is a non-ionic surfactant.

For example, the non-ionic surfactant is selected from the group consisting of polyethylene glycol ether (Brij), poloxamer, polysorbate, sorbitan, and derivatives thereof.

For example, the polyethylene glycol ether is a compound of Formula (VIII):

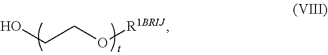

or a salt or isomer thereof, wherein:

t is an integer between 1 and 100;

R1BRIJ independently is C10-40 alkyl, C10-40 alkenyl, or C10-40 alkynyl; and optionally one or more methylene groups of RSPEG are independently replaced with C3-10 carbocyclylene, 4 to 10 membered heterocyclylene, C6-10 arylene, 4 to 10 membered heteroarylene, —N(RN)—, —O—, —S—, —C(O)—, —C(O)N(RN)—, —NRNC(O)—, —NRNC(O)N(RN)—, —C(O)O—, —OC(O)—, —OC(O)O—, —OC(O)N(RN)—, —NRNC(O)O—, —C(O)S—, —SC(O)—, —C(═NRN)—, —C(═NRN)N(RN)—, —NRNC(═NRN)—, —NRNC(═NRN)N(RN)—, —C(S)—, —C(S)N(RN)—, —NRNC(S)—, —NRNC(S)N(RN)—, —S(O)—, —OS(O)—, —S(O)O—, —OS(O)O—, —OS(O)₂—, —S(O)₂O—, —OS(O)₂O—, —N(RN)S(O)—, —S(O)N(RN)—, —N(RN)S(O)N(RN)—, —OS(O)N(RN)—, —N(RN)S(O)O—, —S(O)₂—, —N(RN)S(O)₂—, —S(O)₂N(RN)—, —N(RN)S(O)₂N(RN)—, —OS(O)₂N(RN)—, or —N(RN)S(O)₂O—; and

each instance of RN is independently hydrogen, C1-6 alkyl, or a nitrogen protecting group

In some embodiment, R1BRIJ is C18 alkyl. For example, the polyethylene glycol ether is a compound of Formula (VIII-a):

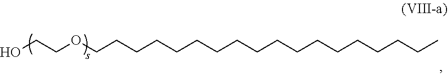

or a salt or isomer thereof.

In some embodiments, R1BRIJ is C18 alkenyl. For example, the polyethylene glycol ether is a compound of Formula (VIII-b):

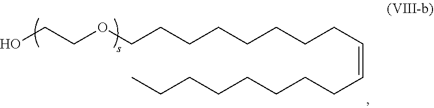

or a salt or isomer thereof

In some embodiments, the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

In some embodiments, the polysorbate is Tween® 20, Tween® 40, Tween®, 60, or Tween® 80.

In some embodiments, the derivative of sorbitan is Span® 20, Span® 60, Span® 65, Span® 80, or Span® 85.

In some embodiments, the concentration of the non-ionic surfactant in the lipid nanoparticle ranges from about 0.00001% w/v to about 1% w/v, e.g., from about 0.00005% w/v to about 0.5% w/v, or from about 0.0001% w/v to about 0.1% w/v.

In some embodiments, the concentration of the non-ionic surfactant in lipid nanoparticle ranges from about 0.000001 wt% to about 1 wt%, e.g., from about 0.000002 wt% to about 0.8 wt%, or from about 0.000005 wt% to about 0.5 wt%.

In some embodiments, the concentration of the PEG lipid in the lipid nanoparticle ranges from about 0.01% by molar to about 50% by molar, e.g., from about 0.05% by molar to about 20% by molar, from about 0.07% by molar to about 10% by molar, from about 0.1% by molar to about 8% by molar, from about 0.2% by molar to about 5% by molar, or from about 0.25% by molar to about 3% by molar.

### Adjuvants

In some embodiments, an LNP of the invention optionally includes one or more adjuvants, e.g., Glucopyranosyl Lipid Adjuvant (GLA), CpG oligodeoxynucleotides (e.g., Class A or B), poly(I:C), aluminum hydroxide, and Pam3CSK4.

### Other Components

An LNP of the invention may optionally include one or more components in addition to those described in the preceding sections. For example, a lipid nanoparticle may include one or more small hydrophobic molecules such as a vitamin (e.g., vitamin A or vitamin E) or a sterol.

Lipid nanoparticles may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, dornase alfa, neltenexine, and erdosteine), and DNases (e.g., rhDNase). A surface altering agent may be disposed within a nanoparticle and/or on the surface of a LNP (e.g., by coating, adsorption, covalent linkage, or other process).

A lipid nanoparticle may also comprise one or more functionalized lipids. For example, a lipid may be functionalized with an alkyne group that, when exposed to an azide under appropriate reaction conditions, may undergo a cycloaddition reaction. In particular, a lipid bilayer may be functionalized in this fashion with one or more groups useful in facilitating membrane permeation, cellular recognition, or imaging. The surface of a LNP may also be conjugated with one or more useful antibodies. Functional groups and conjugates useful in targeted cell delivery, imaging, and membrane permeation are well known in the art.

In addition to these components, lipid nanoparticles may include any substance useful in pharmaceutical compositions. For example, the lipid nanoparticle may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### Methods of Using the Systems or LNP Compositions

The present disclosure provides LNP compositions, which can be delivered to cells, e.g., target cells, e.g., in vitro or in vivo. For in vitro protein expression, the cell is contacted with the LNP by incubating the LNP and the cell ex vivo. Such cells may subsequently be introduced in vivo. For in vivo protein expression, the cell is contacted with the LNP by administering the LNP to a subject to thereby increase or induce protein expression in or on the cells within the subject. For example, in one embodiment, the LNP is administered intravenously. In another embodiment, the LNP is administered intramuscularly. In yet other embodiment, the LNP is administered by a route selected from the group consisting of subcutaneously, intranodally and intratumorally.

For in vitro delivery, in one embodiment the cell is contacted with the LNP by incubating the LNP and the target cell ex vivo. In one embodiment, the cell is a human cell. Various types of cells have been demonstrated to be transfectable by the LNP.

In another embodiment, the cell is contacted with the LNP for, e.g., at least 30 minutes, at least 1 hour, at least 2 hours, at least 3 hours, at least 4 hours, at least 5 hours, at least 6 hours, at least 12 hours or at least 24 hours.

In one embodiment, the cell is contacted with the LNP for a single treatment/transfection. In another embodiment, the cell is contacted with the LNP for multiple treatments/transfections (e.g., two, three, four or more treatments/transfections of the same cells).

In another embodiment, for in vivo delivery, the cell is contacted with the LNP by administering the LNP to a subject to thereby deliver the nucleic acid to cells within the subject. For example, in one embodiment, the LNP is administered intravenously.

In another embodiment, the LNP is administered intramuscularly. In yet other embodiment, the LNP is administered by a route selected from the group consisting of subcutaneously, intranodally and intratumorally.

In an aspect, provided herein is a method of increasing expression of a therapeutic payload or prophylactic payload in a cell, comprising administering to the cell a system, or LNP composition disclosed herein.

In a related aspect, provided herein is a system or LNP composition for use in a method of increasing expression of a therapeutic payload or prophylactic payload in a cell.

In another aspect, the disclosure provides a method of increasing expression of a therapeutic payload or prophylactic payload, in a subject, comprising administering to the subject an effective amount of a system or LNP composition disclosed herein.

In a related aspect, provided herein is a system or LNP composition for use in a method of increasing expression of a therapeutic payload or prophylactic payload in a subject.

In yet another aspect, provided herein is a method of delivering a system, or LNP composition disclosed herein.

In a related aspect, provided herein is a system or LNP composition for use in a method of delivering the system or LNP composition to a cell.

In an embodiment, the method or use, comprises contacting the cell in vitro, in vivo or ex vivo with the system or LNP composition.

In an embodiment, the LNP compositions or systems formulated as LNPs of the present disclosure are contacted with cells, e.g., ex vivo or in vivo and can be used to deliver a secreted polypeptide, an intracellular polypeptide or a transmembrane polypeptide to a subject.

In an aspect, the disclosure provides a method of delivering a system or LNP composition disclosed herein to a subject having a disease or disorder, e.g., as described herein.

In a related aspect, provided herein is a system or LNP composition for use in a method of delivering the system or LNP composition to a subject having a disease or disorder, e.g., as described herein.

In another aspect, provided herein is a method of modulating an immune response in a subject, comprising administering to the subject in need thereof an effective amount of a system, or LNP composition disclosed herein.

In a related aspect, provided herein is a system or LNP composition for use in a method of modulating an immune response in a subject, comprising administering to the subject an effective amount of the system, or LNP composition.

In another aspect, provided herein is a method of delivering a secreted polypeptide, an intracellular polypeptide or a transmembrane polypeptide to a subject.

In an aspect, provided herein is a method of treating, preventing, or preventing a symptom of, a disease or disorder comprising administering to a subject in need thereof an effective amount of a system, or LNP composition disclosed herein.

In a related aspect, provided herein is a system or LNP composition for use in a method of treating, preventing, or preventing a symptom of, a disease or disorder in a subject, comprising administering to the subject in need thereof an effective amount of the system, or LNP composition.

In an embodiment, the first polynucleotide and/or the second polynucleotide of the system is formulated as an LNP. In an embodiment, the first polynucleotide of the system is formulated as an LNP. In an embodiment, the second polynucleotide of the system is formulated as an LNP. In an embodiment, both the first and the second polynucleotides of the system are formulated as LNPs.

In an embodiment, the LNP comprising the first polynucleotide is the same as the LNP comprising the second polynucleotide. In an embodiment, the LNP comprising the first polynucleotide is different from the LNP comprising the second polynucleotide.

In an embodiment, the LNP comprising the first polynucleotide is in a composition. In an embodiment, the LNP comprising the second polynucleotide is in a separate composition. In an embodiment, the LNP comprising the first polynucleotide and the LNP comprising the second polynucleotide are in the same composition.

In an embodiment, the LNP comprising the first polynucleotide and the LNP comprising the second polynucleotide are administered simultaneously, e.g., substantially simultaneously. In some embodiments, the LNP comprising the first polynucleotide and the LNP comprising the second polynucleotide are co-delivered.

In an embodiment, the LNP comprising the first polynucleotide and the LNP comprising the second polynucleotide are administered sequentially.

In an embodiment, the LNP comprising the first polynucleotide is administered first.

In an embodiment, the LNP comprising the first polynucleotide is administered first followed by administration of the LNP comprising the second polynucleotide.

In an embodiment, the LNP comprising the second polynucleotide is administered first.

In an embodiment, the LNP comprising the second polynucleotide is administered first followed by administration of the LNP comprising the first polynucleotide.

In some embodiments, any of the methods or composition for use disclosed herein, results in one, two, three, four, five, six or all, or any combination thereof, of the following in a cell (e.g., in a cell contacted with the system or LNP composition):
(i) increased expression and/or level of mRNA encoding the therapeutic payload or prophylactic payload;
(ii) sustained expression and/or level of mRNA encoding the therapeutic payload or prophylactic payload;
(iii) increased expression and/or level of therapeutic payload or prophylactic payload;
(iv) sustained expression and/or level of therapeutic payload or prophylactic payload;
(v) increased stability of mRNA encoding the therapeutic payload or prophylactic payload;
(vi) increased resistance of translation of therapeutic payload or prophylactic payload to cellular environment, e.g., stress or nutrient deprivation or translation factor availability;
(vii) reduced dosing of the therapeutic payload or prophylactic payload; or
(viii) reduced toxicity, e.g., reduced modulation of a protein translated from endogenous mRNA in a cell.

In some embodiments, the methods or composition for use result in an increased expression and/or level of mRNA encoding the therapeutic payload or prophylactic payload.

In some embodiments, the methods or composition for use result in sustained expression and/or level of mRNA encoding the therapeutic payload or prophylactic payload.

In some embodiments, the methods or composition for use result in increased expression and/or level of therapeutic payload or prophylactic payload.

In some embodiments, the methods or composition for use result in sustained expression and/or level of therapeutic payload or prophylactic payload. In some embodiments, the methods or composition for use result in increased stability of mRNA encoding the therapeutic payload or prophylactic payload;

In some embodiments, the methods or composition for use result in increased resistance of translation of therapeutic payload or prophylactic payload to cellular environment, e.g., stress or nutrient deprivation or translation factor availability.

In some embodiments, the methods or composition for use result in reduced dosing of the therapeutic payload or prophylactic payload.

In some embodiments, the methods or composition for use result in reduced toxicity, e.g., reduced modulation of a protein translated from endogenous mRNA in a cell.

In some embodiments, any one, or all of (i)-(vii) is compared to a cell which:
(a) has not been contacted with the system disclosed herein;
(b) has not been contacted with the LNP composition disclosed herein;
(c) has not been contacted with an LNP comprising the first polynucleotide; or
(d) has been contacted with an LNP comprising the first polynucleotide but has not been contacted with the second polynucleotide, e.g., an LNP comprising the second polynucleotide.

### Combination Therapies

In some embodiments, the methods of treatment or compositions for use disclosed herein, comprise administering an LNP disclosed herein in combination with an additional agent. In an embodiment, the additional agent is a standard of care for the disease or disorder, e.g., autoimmune disease. In an embodiment, the additional agent is an mRNA.

In some aspects, the subject for the present methods or compositions has been treated with one or more standard of care therapies. In other aspects, the subject for the present methods or compositions has not been responsive to one or more standard of care therapies or anti-cancer therapies.

### Sequence Optimization and Methods Thereof

In some embodiments, a polynucleotide of the disclosure comprises a sequence-optimized nucleotide sequence encoding a polypeptide disclosed herein, e.g., a polynucleotide encoding a therapeutic payload or prophylactic payload, an effector molecule and/or a tether molecule. In some embodiments, the polynucleotide of the disclosure comprises an open reading frame (ORF) encoding a therapeutic payload or prophylactic payload, an effector molecule and/or a tether molecule, wherein the ORF has been sequence optimized.

The sequence-optimized nucleotide sequences disclosed herein are distinct from the corresponding wild type nucleotide acid sequences and from other known sequence-optimized nucleotide sequences, e.g., these sequence-optimized nucleic acids have unique compositional characteristics.

In some embodiments, the percentage of uracil or thymine nucleobases in a sequence-optimized nucleotide sequence (e.g., encoding a therapeutic payload or prophylactic payload, an effector molecule and/or a tether molecule, a functional fragment, or a variant thereof) is modified (e.g., reduced) with respect to the percentage of uracil or thymine nucleobases in the reference wild-type nucleotide sequence. Such a sequence is referred to as a uracil-modified or thymine-modified sequence. The percentage of uracil or thymine content in a nucleotide sequence can be determined by dividing the number of uracils or thymines in a sequence by the total number of nucleotides and multiplying by 100. In some embodiments, the sequence-optimized nucleotide sequence has a lower uracil or thymine content than the uracil or thymine content in the reference wild-type sequence. In some embodiments, the uracil or thymine content in a sequence-optimized nucleotide sequence of the disclosure is greater than the uracil or thymine content in the reference wild-type sequence and still maintain beneficial effects, e.g., increased expression and/or signaling response when compared to the reference wild-type sequence.

In some embodiments, the optimized sequences of the present disclosure contain unique ranges of uracils or thymine (if DNA) in the sequence. The uracil or thymine content of the optimized sequences can be expressed in various ways, e.g., uracil or thymine content of optimized sequences relative to the theoretical minimum (%UTM or %TTM), relative to the wild-type (%UWT or %TWT), and relative to the total nucleotide content (%UTL or %TTL). For DNA it is recognized that thymine (T) is present instead of uracil (U), and one would substitute T where U appears. For RNA it is recognized that uracil (U) is present instead of thymine (T). One of skill in the art could readily obtain an RNA sequence when the DNA sequence is provided by substituting thymine in the DNA sequence to uracil. Thus, all the disclosures related to, e.g., %UTM, %UWT, or %UTL, with respect to RNA are equally applicable to %TTM, %TWT, or %TTL with respect to DNA.

Uracil- or thymine-content relative to the uracil or thymine theoretical minimum, refers to a parameter determined by dividing the number of uracils or thymines in a sequence-optimized nucleotide sequence by the total number of uracils or thymines in a hypothetical nucleotide sequence in which all the codons in the hypothetical sequence are replaced with synonymous codons having the lowest possible uracil or thymine content and multiplying by 100. This parameter is abbreviated herein as %UTM or %TTM.

In some embodiments, a uracil-modified sequence encoding a therapeutic payload or prophylactic payload, an effector molecule and/or a tether molecule of the disclosure has a reduced number of consecutive uracils with respect to the corresponding wild-type nucleic acid sequence. For example, two consecutive leucines can be encoded by the sequence CUUUUG, which includes a four uracil cluster. Such a subsequence can be substituted, e.g., with CUGCUC, which removes the uracil cluster. Phenylalanine can be encoded by UUC or UUU. Thus, even if phenylalanines encoded by UUU are replaced by UUC, the synonymous codon still contains a uracil pair (UU). Accordingly, the number of phenylalanines in a sequence establishes a minimum number of uracil pairs (UU) that cannot be eliminated without altering the number of phenylalanines in the encoded polypeptide.

In some embodiments, a uracil-modified sequence encoding a therapeutic payload or prophylactic payload, an effector molecule and/or a tether molecule of the disclosure has a reduced number of uracil triplets (UUU) with respect to the wild-type nucleic acid sequence. In some embodiments, a uracil-modified sequence encoding a therapeutic payload or prophylactic payload, an effector molecule and/or a tether molecule has a reduced number of uracil pairs (UU) with respect to the number of uracil pairs (UU) in the wild-type nucleic acid sequence. In some embodiments, a uracil-modified sequence encoding a therapeutic payload or prophylactic payload, an effector molecule and/or a tether molecule of the disclosure has a number of uracil pairs (UU) corresponding to the minimum possible number of uracil pairs (UU) in the wild-type nucleic acid sequence.

The phrase “uracil pairs (UU) relative to the uracil pairs (UU) in the wild type nucleic acid sequence,” refers to a parameter determined by dividing the number of uracil pairs (UU) in a sequence-optimized nucleotide sequence by the total number of uracil pairs (UU) in the corresponding wild-type nucleotide sequence and multiplying by 100. This parameter is abbreviated herein as %UUwt. In some embodiments, a uracil-modified sequence encoding a therapeutic payload or prophylactic payload, an effector molecule and/or a tether molecule has a %UUwt between below 100%.

In some embodiments, the polynucleotide of the disclosure comprises a uracil-modified sequence encoding an encoding a therapeutic payload or prophylactic payload, an effector molecule and/or a tether molecule disclosed herein. In some embodiments, the uracil-modified sequence encoding a therapeutic payload or prophylactic payload, an effector molecule and/or a tether molecule comprises at least one chemically modified nucleobase, e.g., 5-methoxyuracil. In some embodiments, at least 95% of a nucleobase (e.g., uracil) in a uracil-modified sequence encoding a therapeutic payload or prophylactic payload, an effector molecule and/or a tether molecule of the disclosure are modified nucleobases. In some embodiments, at least 95% of uracil in a uracil-modified sequence encoding a therapeutic payload or prophylactic payload, an effector molecule and/or a tether molecule is 5-methoxyuracil.

In some embodiments, a polynucleotide of the disclosure (e.g., a polynucleotide comprising a nucleotide sequence encoding a therapeutic payload or prophylactic payload, an effector molecule and/or a tether molecule (e.g., the wild-type sequence, functional fragment, or variant thereof) is sequence optimized.

A sequence optimized nucleotide sequence (nucleotide sequence is also referred to as “nucleic acid” herein) comprises at least one codon modification with respect to a reference sequence (e.g., a wild-type sequence encoding a therapeutic payload or prophylactic payload, an effector molecule and/or a tether molecule). Thus, in a sequence optimized nucleic acid, at least one codon is different from a corresponding codon in a reference sequence (e.g., a wild-type sequence).

In general, sequence optimized nucleic acids are generated by at least a step comprising substituting codons in a reference sequence with synonymous codons (i.e., codons that encode the same amino acid). Such substitutions can be effected, for example, by applying a codon substitution map (i.e., a table providing the codons that will encode each amino acid in the codon optimized sequence), or by applying a set of rules (e.g., if glycine is next to neutral amino acid, glycine would be encoded by a certain codon, but if it is next to a polar amino acid, it would be encoded by another codon). In addition to codon substitutions (i.e., “codon optimization”) the sequence optimization methods disclosed herein comprise additional optimization steps which are not strictly directed to codon optimization such as the removal of deleterious motifs (destabilizing motif substitution). Compositions and formulations comprising these sequence optimized nucleic acids (e.g., a RNA, e.g., an mRNA) can be administered to a subject in need thereof to facilitate in vivo expression of functionally active encoding a therapeutic payload or prophylactic payload, an effector molecule and/or a tether molecule.

Additional and exemplary methods of sequence optimization are disclosed in International PCT application WO 2017/201325, filed on 18 May 2017, the entire contents of which are hereby incorporated by reference.

Micro RNA (miRNA) Binding Sites

Nucleic acid molecules (e.g., RNA, e.g., mRNA) of the disclosure can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof.

In some embodiments, a nucleic acid molecule (e.g., RNA, e.g., mRNA) of the disclosure comprises an open reading frame (ORF) encoding a polypeptide of interest and further comprises one or more miRNA binding site(s). Inclusion or incorporation of miRNA binding site(s) provides for regulation of nucleic acid molecules (e.g., RNA, e.g., mRNA) of the disclosure, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a nucleic acid molecule (e.g., RNA, e.g., mRNA) and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, a nucleic acid molecule (e.g., RNA, e.g., mRNA) of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within a nucleic acid molecule, e.g., within a DNA or within an RNA transcript, including in the 5′UTR and/or 3′UTR, that has sufficient complementarity to all or a region of a miRNA to interact with, associate with or bind to the miRNA. In some embodiments, a nucleic acid molecule (e.g., RNA, e.g., mRNA) of the disclosure comprising an ORF encoding a polypeptide of interest and further comprises one or more miRNA binding site(s). In exemplary embodiments, a 5′UTR and/or 3′UTR of the nucleic acid molecule (e.g., RNA, e.g., mRNA) comprises the one or more miRNA binding site(s).

A miRNA binding site having sufficient complementarity to a miRNA refers to a degree of complementarity sufficient to facilitate miRNA-mediated regulation of a nucleic acid molecule (e.g., RNA, e.g., mRNA), e.g., miRNA-mediated translational repression or degradation of the nucleic acid molecule (e.g., RNA, e.g., mRNA). In exemplary aspects of the disclosure, a miRNA binding site having sufficient complementarity to the miRNA refers to a degree of complementarity sufficient to facilitate miRNA-mediated degradation of the nucleic acid molecule (e.g., RNA, e.g., mRNA), e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the nucleic acid molecule (e.g., RNA, e.g., mRNA) comprising the miRNA binding site. In other embodiments, the miRNA binding site has imperfect complementarity so that a RISC complex comprising the miRNA induces instability in the nucleic acid molecule (e.g., RNA, e.g., mRNA) comprising the miRNA binding site. In another embodiment, the miRNA binding site has imperfect complementarity so that a RISC complex comprising the miRNA represses transcription of the nucleic acid molecule (e.g., RNA, e.g., mRNA) comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a nucleic acid molecule (e.g., RNA, e.g., mRNA) of the disclosure, the nucleic acid molecule (e.g., RNA, e.g., mRNA) can be targeted for degradation or reduced translation, provided the miRNA in question is available. This can reduce off-target effects upon delivery of the nucleic acid molecule (e.g., RNA, e.g., mRNA). For example, if a nucleic acid molecule (e.g., RNA, e.g., mRNA) of the disclosure is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the nucleic acid molecule (e.g., RNA, e.g., mRNA).

For example, one of skill in the art would understand that one or more miR binding sites can be included in a nucleic acid molecule (e.g., an RNA, e.g., mRNA) to minimize expression in cell types other than lymphoid cells. In one embodiment, a miR122 binding site can be used. In another embodiment, a miR126 binding site can be used. In still another embodiment, multiple copies of these miR binding sites or combinations may be used.

Conversely, miRNA binding sites can be removed from nucleic acid molecule (e.g., RNA, e.g., mRNA) sequences in which they naturally occur in order to increase protein expression in specific tissues. For example, a binding site for a specific miRNA can be removed from a nucleic acid molecule (e.g., RNA, e.g., mRNA) to improve protein expression in tissues or cells containing the miRNA.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/1eu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and monocytes), monocytes, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation. Immune cell specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a nucleic acid molecule (e.g., RNA, e.g., mRNA) can be shut-off by adding miR-142 binding sites to the 3′-UTR of the polynucleotide, enabling more stable gene transfer in tissues and cells. miR-142 efficiently degrades exogenous nucleic acid molecules (e.g., RNA, e.g., mRNA) in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of a nucleic acid molecule of the disclosure can selectively repress gene expression in antigen presenting cells through miR-142 mediated degradation, limiting antigen presentation in antigen presenting cells (e.g., dendritic cells) and thereby preventing antigen-mediated immune response after the delivery of the nucleic acid molecule (e.g., RNA, e.g., mRNA). The nucleic acid molecule (e.g., RNA, e.g., mRNA) is then stably expressed in target tissues or cells without triggering cytotoxic elimination.

In one embodiment, binding sites for miRNAs that are known to be expressed in immune cells, in particular, antigen presenting cells, can be engineered into a nucleic acid molecule (e.g., RNA, e.g., mRNA) of the disclosure to suppress the expression of the nucleic acid molecule (e.g., RNA, e.g., mRNA) in antigen presenting cells through miRNA mediated RNA degradation, subduing the antigen-mediated immune response. Expression of the nucleic acid molecule (e.g., RNA, e.g., mRNA) is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of a nucleic acid molecule of the disclosure.

To further drive the selective degradation and suppression in APCs and macrophage, a nucleic acid molecule (e.g., RNA, e.g., mRNA) of the disclosure can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2--5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p,miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p,miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11,288, the content of each of which is incorporated herein by reference in its entirety.)

In some embodiments, a miRNA binding site is inserted in the nucleic acid molecule (e.g., RNA, e.g., mRNA) of the disclosure in any position of the nucleic acid molecule (e.g., RNA, e.g., mRNA) (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the nucleic acid molecule (e.g., RNA, e.g., mRNA) can be anywhere in the nucleic acid molecule (e.g., RNA, e.g., mRNA) as long as the insertion of the miRNA binding site in the nucleic acid molecule (e.g., RNA, e.g., mRNA) does not interfere with the translation of a functional polypeptide in the absence of the corresponding miRNA; and in the presence of the miRNA, the insertion of the miRNA binding site in the nucleic acid molecule (e.g., RNA, e.g., mRNA) and the binding of the miRNA binding site to the corresponding miRNA are capable of degrading the polynucleotide or preventing the translation of the nucleic acid molecule (e.g., RNA, e.g., mRNA).

In some embodiments, a miRNA binding site is inserted in at least about 30 nucleotides downstream from the stop codon of an ORF in a nucleic acid molecule (e.g., RNA, e.g., mRNA) of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a nucleic acid molecule (e.g., RNA, e.g., mRNA) of the disclosure.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The nucleic acid molecules (e.g., RNA, e.g., mRNA) of the disclosure can further include this structured 5′UTR in order to enhance microRNA mediated gene regulation.

At least one miRNA binding site can be engineered into the 3′UTR of a polynucleotide of the disclosure. In this context, at least two, at least three, at least four, at least five, at least six, at least seven, at least eight, at least nine, at least ten, or more miRNA binding sites can be engineered into a 3′UTR of a nucleic acid molecule (e.g., RNA, e.g., mRNA) of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a nucleic acid molecule (e.g., RNA, e.g., mRNA) of the disclosure. In one embodiment, miRNA binding sites incorporated into a nucleic acid molecule (e.g., RNA, e.g., mRNA) of the disclosure can be the same or can be different miRNA sites. A combination of different miRNA binding sites incorporated into a nucleic acid molecule (e.g., RNA, e.g., mRNA) of the disclosure can include combinations in which more than one copy of any of the different miRNA sites are incorporated. In another embodiment, miRNA binding sites incorporated into a nucleic acid molecule (e.g., RNA, e.g., mRNA) of the disclosure can target the same or different tissues in the body. As a non-limiting example, through the introduction of tissue-, cell-type-, or disease-specific miRNA binding sites in the 3′-UTR of a nucleic acid molecule (e.g., RNA, e.g., mRNA) of the disclosure, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a nucleic acid molecule (e.g., RNA, e.g., mRNA) of the disclosure. As a non-limiting example, a miRNA binding site can be engineered near the 5′ terminus of the 3′ UTR and about halfway between the 5′ terminus and 3′ terminus of the 3′ UTR. As another non-limiting example, a miRNA binding site can be engineered near the 3′ terminus of the 3′UTR and about halfway between the 5′ terminus and 3′ terminus of the 3′ UTR. As yet another non-limiting example, a miRNA binding site can be engineered near the 5′ terminus of the 3′ UTR and near the 3′ terminus of the 3′ UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

A nucleic acid molecule (e.g., RNA, e.g., mRNA) of the disclosure can be engineered for more targeted expression in specific tissues, cell types, or biological conditions based on the expression patterns of miRNAs in the different tissues, cell types, or biological conditions. Through introduction of tissue-specific miRNA binding sites, a nucleic acid molecule (e.g., RNA, e.g., mRNA) of the disclosure can be designed for optimal protein expression in a tissue or cell, or in the context of a biological condition.

In some embodiments, a nucleic acid molecule (e.g., RNA, e.g., mRNA) of the disclosure can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make a nucleic acid molecule (e.g., RNA, e.g., mRNA) of the disclosure more unstable in antigen presenting cells. Non-limiting examples of these miRNAs include mir-142-5p, mir-142-3p, mir-146a-5p, and mir-146-3p

### IVT Polynucleotide Architecture

In some embodiments, the polynucleotide of the present disclosure comprising an mRNA encoding a therapeutic payload or prophylactic payload, an effector molecule and/or a tether molecule is an IVT polynucleotide. Traditionally, the basic components of an mRNA molecule include at least a coding region, a 5′UTR, a 3′UTR, a 5′ cap and a poly-A tail. The IVT polynucleotides of the present disclosure can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded therapeutic payload or prophylactic payload, an effector molecule and/or a tether molecule. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding a therapeutic payload or prophylactic payload, an effector molecule and/or a tether molecule can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of a therapeutic payload or prophylactic payload, an effector molecule and/or a tether molecule or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Additional and exemplary features of IVT polynucleotide architecture are disclosed in International PCT application WO 2017/201325, filed on 18 May 2017, the entire contents of which are hereby incorporated by reference.

### 5′ UTR and 3′ UTR

A UTR can be homologous or heterologous to the coding region in a polynucleotide. In some embodiments, the UTR is homologous to the ORF encoding the therapeutic payload or prophylactic payload, an effector molecule and/or a tether molecule. In some embodiments, the UTR is heterologous to the ORF encoding the therapeutic payload or prophylactic payload, an effector molecule and/or a tether molecule.

In some embodiments, the polynucleotide comprises two or more 5′ UTRs or functional fragments thereof, each of which has the same or different nucleotide sequences. In some embodiments, the polynucleotide comprises two or more 3′ UTRs or functional fragments thereof, each of which has the same or different nucleotide sequences.

In some embodiments, the 5′ UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., N1-methylpseudouracil or 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG (SEQ ID NO:157), where R is a purine (adenine or guanine) three bases upstream of the start codon (AUG), which is followed by another ‘G’. 5′ UTRs also have been known to form secondary structures that are involved in elongation factor binding.

By engineering the features typically found in abundantly expressed genes of specific target organs, one can enhance the stability and protein production of a polynucleotide. For example, introduction of 5′ UTR of liver-expressed mRNA, such as albumin, serum amyloid A, Apolipoprotein AB/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Exemplary UTRs of the application include, but are not limited to, one or more 5′UTR and/or 3¹UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human a or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a (3-Fl-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nntl); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′UTR containing a strong Kozak translational initiation signal; a cytochrome b-245 α polypeptide (CYBA) 5′ UTR; a hydroxysteroid (1743) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′UTR; a DEN 3′ UTR; a PAV barley yellow dwarf virus (BYDV-PAV) 3′ UTR; an elongation factor 1 α1 (EEF1A1) 3′ UTR; a manganese superoxide dismutase (MnSOD) 3′ UTR; a β subunit of mitochondrial H(+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the polynucleotides of the invention. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety.

UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′ UTR or 3′ UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the polynucleotides of the invention comprise a 5′ UTR and/or a 3′ UTR selected from any of the UTRs disclosed herein, e.g., in Table 5.

TABLE 5
Exemplary 5′ UTR and 3′ UTR sequences

| | |
|---|---|
| SEQ ID NO: | Sequence |

3′ UTR sequences

| | |
|---|---|
| 99 | GCUGGAGCCUCGGUGGCCAUGCUUCUUGCCCCUUGGGCCUCCCCCCAGC |
| | CCCUCCUCCCCUUCCUGCACCCGUACCCCCUCCAUAAAGUAGGAAACAC |
| | UACAGUGGUCUUUGAAUAAAGUCUGAGUGGGCGGC |
| | (3′ UTR with miR 142-3p binding site) |
| 100 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCC [symbol] |
| | [symbol] [symbol] GUGGUCUUUGAAUAAAGUCUGAGUGGGCGGC |
| | (3′ UTR with miR 126-3p binding site) |
| 101 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCGUGGUCUUU |
| | GAAUAAAGUCUGAGUGGGCGGC |
| | (3′ UTR, no miR binding sites) |
| 102 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCUCCAUAAAG |
| | UAGGAAACACUACAGUGGUCUUUGAAUAAAGUCUGAGUGGGCGGC |
| | (3′ UTR with miR 142-3p binding site) |
| 103 | UGAUAAUAGUCCAUAAAGUAGGAAACACUACAGCUGGAGCCUCGGUGG |
| | CCAUGCUUCUUGCCCCUUGGGCCUCCCCCCAGCCCCUCCUCCCCUUCCU |
| | GCACCCGUACCCCC [symbol] [symbol] [symbol] GUGGUCUUUGAA |
| | UAAAGUCUGAGUGGGCGGC |
| | (3′ UTR with miR 142-3p and miR 126-3p binding |
| | sites variant 1) |
| 104 | UGAUAAUAGUCCAUAAAGUAGGAAACACUACAGCUGGAGCCUCGGUGG |
| | CCAUGCUUCUUGCCCCUUGGGCCUCCAUAAAGUAGGAAACACUACAUC |
| | CCCCCAGCCCCUCCUCCCCUUCCUGCACCCGUACCCCCUCCAUAAAGUA |
| | GGAAACACUACAGUGGUCUUUGAAUAAAGUCUGAGUGGGCGGC |
| | (3′ UTR with 3 miR 142-3p binding sites) |
| 105 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCC [symbol] [symbol] |
| | [symbol] [symbol] GUGGUCUUUGAAUAAAGUCUGAGUGGGCGGC |
| | (3′ UTR with miR 142-5p binding site) |
| 106 | UGAUAAUAG [symbol] [symbol] [symbol] GCUGGAGCCUCGGUGGCC |
| | AUGCUUCUUGCCCCUUGGGCC [symbol] [symbol] [symbol] UCCCCCC |
| | AGCCCCUCCUCCCCUUCCUGCACCCGUACCCCC [symbol] [symbol] |
| | [symbol] GUGGUCUUUGAAUAAAGUCUGAGUGGGCGGC |
| | (3′ UTR with 3 miR 142-5p binding sites) |
| 107 | UGAUAAUAG [symbol] [symbol] [symbol] GCUGGAGCCUCGGUGGCC |
| | AUGCUUCUUGCCCCUUGGGCCUCCAUAAAGUAGGAAACACUACAUCCC |
| | CCCAGCCCCUCCUCCCCUUCCUGCACCCGUACCCCC [symbol] [symbol] |
| | [symbol] GUGGUCUUUGAAUAAAGUCUGAGUGGGCGGC |
| | (3′ UTR with 2 miR 142-5p binding sites and 1 |
| | miR 142-3p binding site) |
| 108 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCACCCCUAUCA |
| | CAAUUAGCAUUAAGUGGUCUUUGAAUAAAGUCUGAGUGGGCGGC |
| | (3′ UTR with miR 155-5p binding site) |
| 109 | UGAUAAUAGACCCCUAUCACAAUUAGCAUUAAGCUGGAGCCUCGGUGGC |
| | CAUGCUUCUUGCCCCUUGGGCCACCCCUAUCACAAUUAGCAUUAAUCCCC |
| | CCAGCCCCUCCUCCCCUUCCUGCACCCGUACCCCCACCCCUAUCACAAUU |
| | AGCAUUAAGUGGUCUUUGAAUAAAGUCUGAGUGGGCGGC |
| | (3′ UTR with 3 miR 155-5p binding sites) |
| 110 | UGAUAAUAGACCCCUAUCACAAUUAGCAUUAAGCUGGAGCCUCGGUGGC |
| | CAUGCUUCUUGCCCCUUGGGCCUCCAUAAAGUAGGAAACACUACAUCC |
| | CCCCAGCCCCUCCUCCCCUUCCUGCACCCGUACCCCCACCCCUAUCACAA |
| | UUAGCAUUAAGUGGUCUUUGAAUAAAGUCUGAGUGGGCGGC |
| | (3′ UTR with 2 miR 155-5p binding sites and 1 |
| | miR 142-3p binding site) |
| 111 | UGAUAAUAGUCCAUAAAGUAGGAAACACUACAGCUGGAGCCUCGGUGG |
| | CCAUGCUUCUUGCCCCUUGGGCCUCCCCCCAGCCCCUCCUCCCCUUCCU |
| | GCACCCGUACCCCCGUGGUCUUUGAAUAAAGUCUGAGUGGGCGGC |
| | (3′ UTR with miR 142-3p binding site, P1 insertion) |
| 112 | UGAUAAUAGGCUGGAGCCUCGGUGGCUCCAUAAAGUAGGAAACACUAC |
| | ACAUGCUUCUUGCCCCUUGGGCCUCCCCCCAGCCCCUCCUCCCCUUCCU |
| | GCACCCGUACCCCCGUGGUCUUUGAAUAAAGUCUGAGUGGGCGGC |
| | (3′ UTR with miR 142-3p binding site, P2 insertion) |
| 113 | UGAUAAUAGGCUGGAGCCUCGGUGGCCAUGCUUCUUGCCCCUUGGGCC |
| | UCCAUAAAGUAGGAAACACUACAUCCCCCCAGCCCCUCCUCCCCUUCCU |
| | GCACCCGUACCCCCGUGGUCUUUGAAUAAAGUCUGAGUGGGCGGC |
| | (3′ UTR with miR 142-3p binding site, P3 insertion) |
| 114 | UGAUAAUAG [symbol] [symbol] [symbol] GCUGGAGCCUCGGUGGCC |
| | AUGCUUCUUGCCCCUUGGGCC [symbol] [symbol] [symbol] UCCCCCC |
| | AGCCCCGCGCCCCGGCCGGCACCCGGACCCCC [symbol] [symbol] |
| | [symbol] GUGGUCUUUGAAUAAAGUCUGAGUGGGCGGC |
| | (3′ UTR with 3 miR 142-5p binding sites) |
| 115 | UGAUAAUAGGCUGGAGCCUCGGUGGCCAUGCUUCUUGCCCCUUCCAUA |
| | AAGUAGGAAACACUACAUGGGCCUCCCCCCAGCCCCUCCUCCCCUUCCU |
| | GCACCCGUACCCCCGUGGUCUUUGAAUAAAGUCUGAGUGGGCGGC |
| | (3′ UTR including miR 142-3p binding site) |
| 116 | UGAUAAUAGGCUGGAGCCUCGGUGGCCAUGCUUCUUGCCCCUUGGGCC |
| | UCCCCCCAGUCCAUAAAGUAGGAAACACUACACCCCUCCUCCCCUUCCU |
| | GCACCCGUACCCCCGUGGUCUUUGAAUAAAGUCUGAGUGGGCGGC |
| | (3′ UTR including miR 142-3p binding site) |
| 117 | UGAUAAUAGGCUGGAGCCUCGGUGGCCAUGCUUCUUGCCCCUUGGGCC |
| | UCCCCCCAGCCCCUCCUCCCCUUCUCCAUAAAGUAGGAAACACUACACU |
| | GCACCCGUACCCCCGUGGUCUUUGAAUAAAGUCUGAGUGGGCGGC |
| | (3′ UTR including miR 142-3p binding site) |
| 118 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCGUGGUCUUU |
| | GAAUAAAGUUCCAUAAAGUAGGAAACACUACACUGAGUGGGCGGC |
| | (3′ UTR including miR 142-3p binding site) |
| 119 | UGAUAAUAGUCCAUAAAGUAGGAAACACUACAGCUGGAGCCUCGGUGG |
| | CCUAGCUUCUUGCCCCUUGGGCCUCCCCCCAGCCCCUCCUCCCCUUCCU |
| | GCACCCGUACCCCC [symbol] [symbol] [symbol] GUGGUCUUUGAA |
| | UAAAGUCUGAGUGGGCGGC |
| | (3′ UTR with miR 142-3p and miR 126-3p binding |
| | sites variant 2) |
| 120 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCCGUGGUCUUU |
| | GAAUAAAGUCUGAGUGGGCGGC |
| | (3′ UTR, no miR binding sites variant 2) |
| 121 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCCUCCAUAAAG |
| | UAGGAAACACUACAGUGGUCUUUGAAUAAAGUCUGAGUGGGCGGC |
| | (3′ UTR with miR 142-3p binding site variant 3) |
| 122 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCC [symbol] |
| | [symbol] [symbol] GUGGUCUUUGAAUAAAGUCUGAGUGGGCGGC |
| | (3′ UTR with miR 126-3p binding site variant 3) |
| 123 | UGAUAAUAGUCCAUAAAGUAGGAAACACUACAGCUGGAGCCUCGGUGG |
| | CCUAGCUUCUUGCCCCUUGGGCCUCCAUAAAGUAGGAAACACUACAUC |
| | CCCCCAGCCCCUCCUCCCCUUCCUGCACCCGUACCCCCUCCAUAAAGUA |
| | GGAAACACUACAGUGGUCUUUGAAUAAAGUCUGAGUGGGCGGC |
| | (3′ UTR with 3 miR 142-3p binding sites variant 2) |
| 124 | UGAUAAUAGUCCAUAAAGUAGGAAACACUACAGCUGGAGCCUCGGUGG |
| | CCUAGCUUCUUGCCCCUUGGGCCUCCCCCCAGCCCCUCCUCCCCUUCCU |
| | GCACCCGUACCCCCGUGGUCUUUGAAUAAAGUCUGAGUGGGCGGC |
| | (3′ UTR with miR 142-3p binding site, P1 insertion |
| | variant 2) |
| 125 | UGAUAAUAGGCUGGAGCCUCGGUGGCUCCAUAAAGUAGGAAACACUAC |
| | ACUAGCUUCUUGCCCCUUGGGCCUCCCCCCAGCCCCUCCUCCCCUUCCU |
| | GCACCCGUACCCCCGUGGUCUUUGAAUAAAGUCUGAGUGGGCGGC |
| | (3′ UTR with miR 142-3p binding site, P2 insertion |
| | variant 2) |
| 126 | UGAUAAUAGGCUGGAGCCUCGGUGGCCUAGCUUCUUGCCCCUUGGGCC |
| | UCCAUAAAGUAGGAAACACUACAUCCCCCCAGCCCCUCCUCCCCUUCCU |
| | GCACCCGUACCCCCGUGGUCUUUGAAUAAAGUCUGAGUGGGCGGC |
| | (3′ UTR with miR 142-3p binding site, P3 insertion |
| | variant 2) |
| 127 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCCACCCCUAUCA |
| | CAAUUAGCAUUAAGUGGUCUUUGAAUAAAGUCUGAGUGGGCGGC |
| | (3′ UTR with miR 155-5p binding site variant 2) |
| 128 | UGAUAAUAGACCCCUAUCACAAUUAGCAUUAAGCUGGAGCCUCGGUGGC |
| | CUAGCUUCUUGCCCCUUGGGCCACCCCUAUCACAAUUAGCAUUAAUCCCC |
| | CCAGCCCCUCCUCCCCUUCCUGCACCCGUACCCCCACCCCUAUCACAAUU |
| | AGCAUUAAGUGGUCUUUGAAUAAAGUCUGAGUGGGCGGC |
| | (3′ UTR with 3 miR 155-5p binding sites variant 2) |
| 129 | UGAUAAUAGACCCCUAUCACAAUUAGCAUUAAGCUGGAGCCUCGGUGGC |
| | CUAGCUUCUUGCCCCUUGGGCCUCCAUAAAGUAGGAAACACUACAUCC |
| | CCCCAGCCCCUCCUCCCCUUCCUGCACCCGUACCCCCACCCCUAUCACAA |
| | UUAGCAUUAAGGGGGCGGGGAAGAAAGGCGGAGGGGGCGGC |
| | (3′ UTR with 2 miR 155-5p binding sites and 1 |
| | miR 142-3p binding site variant 2) |
| 130 | UGAUAAUAGGCUGGAGCCUCGGUGGCCAUGCUUCUUGCCCCUUGGGCC |
| | UCCCCCCAGCCCCUCCUCCCCUUCCUGCACCCGUACCCCCCAAACACCAU |
| | UGUCACACUCCAGUGGUCUUUGAAUAAAGUCUGAGUGGGCGGC |
| | (3′ UTR (MIR122)) |
| 131 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCCCAAACACCAU |
| | UGUCACACUCCAGUGGUCUUUGAAUAAAGUCUGAGUGGGCGGC |
| | 3′ UTR (MIR122-TAG) |
| 132 | UGAUAAUAGGCUGGAGCCUCGGUGGCCUAGCUUCUUGCCCCUUGGGCC |
| | UCCCCCCAGCCCCUCCUCCCCUUCCUGCACCCGUACCCCCCGCAUUAUU |
| | ACUCACGGUACGAGUGGUCUUUGAAUAAAGUCUGAGUGGGCGGC |
| | (3′ UTR V1.1 (MIR126-3P)) |

5′ UTR SEQUENCES

| | |
|---|---|
| 133 | GGGAAAUAAGAGAGAAAAGAAGAGUAAGAAGAAAUAUAAGAGCCACC |
| | (5′ UTR) |
| 134 | GGGAAAUAAGAGUCCAUAAAGUAGGAAACACUACAAGAAAAGAAGAGU |
| | AAGAAGAAAUAUAAGAGCCACC |
| | (5′ UTR with miR142-3p binding site at position p1) |
| 135 | GGGAAAUAAGAGAGAAAAGAAGAGUAAUCCAUAAAGUAGGAAACACU |
| | ACAGAAGAAAUAUAAGAGCCACC |
| | (5′ UTR with miR142-3p binding site at position p2) |
| 136 | GGGAAAUAAGAGAGAAAAGAAGAGUAAGAAGAAAUAUAAUCCAUAAA |
| | GUAGGAAACACUACAGAGCCACC |
| | (5′ UTR with miR142-3p binding site at position p3) |
| 137 | AGGAAAUAAGAGAGAAAAGAAGAGUAAGAAGAAAUAUAAGAGCCACC |
| | 5′-UTR (v1 A-start) |
| 138 | GGGAAAUAAGAGAGAAAAGAAGAGUAAGAAGAAAUAUAAGACCCCGGC |
| | GCCGCCACC |
| | (5′ UTR v1.1) |
| 139 | AGGAAAUAAGAGAGAAAAGAAGAGUAAGAAGAAAUAUAAGACCCCGGC |
| | GCCGCCACC |
| | (5′ UTR v1.1 A-start) |
| 140 | GGGAGAUCAGAGAGAAAAGAAGAGUAAGAAGAAAUAUAAGAGCCACC |
| | 5′ UTR 002 (upstream UTR) |
| 141 | GGGAGACAAGCUUGGCAUUCCGGUACUGUUGGUAAAGCCACC |
| | 5′ UTR-004 (Upstream UTR) |
| 142 | GGGAAUUAACAGAGAAAAGAAGAGUAAGAAGAAAUAUAAGAGCCACC |
| | 5′ UTR-008 (Upstream UTR) |
| 143 | GGGAAAUUAGACAGAAAAGAAGAGUAAGAAGAAAUAUAAGAGCCACC |
| | 5′ UTR-009 (Upstream UTR) |
| 144 | GGGAAAUAAGAGAGUAAAGAACAGUAAGAAGAAAUAUAAGAGCCACC |
| | 5′ UTR-010, Upstream |
| 145 | GGGAAAAAAGAGAGAAAAGAAGACUAAGAAGAAAUAUAAGAGCCACC |
| | 5′ UTR-011 (Upstream UTR) |
| 146 | GGGAAAUAAGAGAGAAAAGAAGAGUAAGAAGAUAUAUAAGAGCCACC |
| | 5′ UTR-012 (Upstream UTR) |
| 147 | GGGAAAUAAGAGACAAAACAAGAGUAAGAAGAAAUAUAAGAGCCACC |
| | 5′ UTR-013 (Upstream UTR) |
| 148 | GGGAAAUUAGAGAGUAAAGAACAGUAAGUAGAAUUAAAAGAGCCACC |
| | 5′ UTR-014 (Upstream UTR) |
| 149 | GGGAAAUAAGAGAGAAUAGAAGAGUAAGAAGAAAUAUAAGAGCCACC |
| | 5′ UTR-015 (Upstream UTR) |
| 150 | GGGAAAUAAGAGAGAAAAGAAGAGUAAGAAGAAAAUUAAGAGCCACC |
| | 5′ UTR-016 (Upstream UTR) |
| 151 | GGGAAAUAAGAGAGAAAAGAAGAGUAAGAAGAAAUUUAAGAGCCACC |
| | 5′ UTR-017 (Upstream UTR) |
| 152 | UCAAGCUUUUGGACCCUCGUACAGAAGCUAAUACGACUCACUAUAGGG |
| | AAAUAAGAGAGAAAAGAAGAGUAAGAAGAAAUAUAAGAGCCACC |
| | 5′ UTR-018 (Upstream UTR) |

Stop codon = bold
miR 142-3p binding site = underline
miR 126-3p binding site = bold underline
miR 155-5p binding site = italicized
miR 142-5p binding site = italicized and bold underline

In certain embodiments, the 5′ UTR and/or 3′ UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence provided in Table 5.

In some embodiments, the 5′ UTR comprises a nucleotide sequence at least about 60%, at least about 70%, at least about 80%, at least about 90%, at least about 95%, at least about 96%, at least about 97%, at least about 98%, at least about 99%, or about 100% identical to a sequence provided in Table 5.

In some embodiments, the 3′ UTR comprises a nucleotide sequence at least about 60%, at least about 70%, at least about 80%, at least about 90%, at least about 95%, at least about 96%, at least about 97%, at least about 98%, at least about 99%, or about 100% identical to a sequence provided in Table 5.

In some embodiments, the polynucleotide disclosed herein, e.g., the polynucleotide encoding a therapeutic payload or prophylactic payload, an effector molecule and/or a tether molecule, comprises a 5′ UTR having the sequence of a 5′ UTR provided in Table 5, or a sequence with at least about 60%, at least about 70%, at least about 80%, at least about 90%, at least about 95%, at least about 96%, at least about 97%, at least about 98%, at least about 99%, or about 100% identity thereto. In some embodiments, the polynucleotide comprises a 5′ UTR comprising the sequence of any one of SEQ ID NOs: 133, 134, 135, 136, 137, 138, 139, 140, 141, 142, 143, 144, 145, 146, 147, 148, 149, 150, 151, or 152, ora sequence with at least about 60%, at least about 70%, at least about 80%, at least about 90%, at least about 95%, at least about 96%, at least about 97%, at least about 98%, at least about 99%, or about 100% identity thereto.

In some embodiments, the polynucleotide disclosed herein, e.g., the polynucleotide encoding a therapeutic payload or prophylactic payload, an effector molecule and/or a tether molecule, comprises a 3′ UTR having the sequence of a 3′ UTR provided in Table 5, or a sequence with at least about 60%, at least about 70%, at least about 80%, at least about 90%, at least about 95%, at least about 96%, at least about 97%, at least about 98%, at least about 99%, or about 100% identity thereto. In some embodiments, the polynucleotide comprises a 3′ UTR comprising the sequence of any one of SEQ ID NOs: 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, or 132, or a sequence with at least about 60%, at least about 70%, at least about 80%, at least about 90%, at least about 95%, at least about 96%, at least about 97%, at least about 98%, at least about 99%, or about 100% identity thereto.

The polynucleotides of the invention can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

Other non-UTR sequences can be used as regions or subregions within the polynucleotides of the invention. For example, introns or portions of intron sequences can be incorporated into the polynucleotides of the invention. Incorporation of intronic sequences can increase protein production as well as polynucleotide expression levels. In some embodiments, the polynucleotide of the invention comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises an IRES instead of a 5′ UTR sequence. In some embodiments, the polynucleotide comprises an ORF and a viral capsid sequence. In some embodiments, the polynucleotide comprises a synthetic 5′ UTR in combination with a non-synthetic 3′ UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation.

Regions having a 5′ Cap

The disclosure also includes a polynucleotide that comprises both a 5′ Cap and a polynucleotide of the present invention (e.g., a polynucleotide comprising a nucleotide sequence encoding a therapeutic payload or prophylactic payload, an effector molecule and/or a tether molecule).

The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns during mRNA splicing.

Endogenous mRNA molecules can be 5′-end capped generating a 5′-ppp-5′-triphosphate linkage between a terminal guanosine cap residue and the 5′-terminal transcribed sense nucleotide of the mRNA molecule. This 5′-guanylate cap can then be methylated to generate an N7-methyl-guanylate residue. The ribose sugars of the terminal and/or ante-terminal transcribed nucleotides of the 5′ end of the mRNA can optionally also be 2′-O-methylated. 5′-decapping through hydrolysis and cleavage of the guanylate cap structure can target a nucleic acid molecule, such as an mRNA molecule, for degradation.

In some embodiments, the polynucleotides of the present invention (e.g., a polynucleotide comprising a nucleotide sequence encoding a therapeutic payload or prophylactic payload, an effector molecule and/or a tether molecule) incorporate a cap moiety.

In some embodiments, polynucleotides of the present invention (e.g., a polynucleotide comprising a nucleotide sequence encoding a therapeutic payload or prophylactic payload, an effector molecule and/or a tether molecule) comprise a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with a-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide that functions as an mRNA molecule. Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ O—Me-m7G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110, the contents of which are herein incorporated by reference in its entirety.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog (See, e.g., the various cap analogs and the methods of synthesizing cap analogs described in Kore et al. Bioorganic & Medicinal Chemistry 2013 21:4570-4574; the contents of which are herein incorporated by reference in its entirety). In another embodiment, a cap analog of the present invention is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

Polynucleotides of the invention (e.g., a polynucleotide comprising a nucleotide sequence encoding a therapeutic payload or prophylactic payload, an effector molecule and/or a tether molecule) can also be capped post-manufacture (whether IVT or chemical synthesis), using enzymes, to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those that, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)N1mpNp (cap 1), and 7mG(5′)-ppp(5′)N1mpN2mp (cap 2).

As a non-limiting example, capping chimeric polynucleotides post-manufacture can be more efficient as nearly 100% of the chimeric polynucleotides can be capped.

This is in contrast to —80% efficiency when a cap analog is linked to a chimeric polynucleotide during an in vitro transcription reaction.

According to the present invention, 5′ terminal caps can include endogenous caps or cap analogs. According to the present invention, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Poly A Tails

In some embodiments, the polynucleotides of the present disclosure (e.g., a polynucleotide comprising a nucleotide sequence encoding a therapeutic payload or prophylactic payload, an effector molecule and/or a tether molecule) further comprise a poly-A tail. In further embodiments, terminal groups on the poly-A tail can be incorporated for stabilization. In other embodiments, a poly-A tail comprises des-3′ hydroxyl tails.

During RNA processing, a long chain of adenine nucleotides (poly-A tail) can be added to a polynucleotide such as an mRNA molecule to increase stability. Immediately after transcription, the 3′ end of the transcript can be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long. In one embodiment, the poly-A tail is 100 nucleotides in length (SEQ ID NO:84).

(SEQ ID NO: 84)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa

PolyA tails can also be added after the construct is exported from the nucleus.

According to the present invention, terminal groups on the poly A tail can be incorporated for stabilization. Polynucleotides of the present invention can include des-3′ hydroxyl tails. They can also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides of the present invention can be designed to encode transcripts with alternative polyA tail structures including histone mRNA. According to Norbury, “Terminal uridylation has also been detected on human replication-dependent histone mRNAs. The turnover of these mRNAs is thought to be important for the prevention of potentially toxic histone accumulation following the completion or inhibition of chromosomal DNA replication. These mRNAs are distinguished by their lack of a 3′ poly(A) tail, the function of which is instead assumed by a stable stem—loop structure and its cognate stem—loop binding protein (SLBP); the latter carries out the same functions as those of PABP on polyadenylated mRNAs” (Norbury, “Cytoplasmic RNA: a case of the tail wagging the dog,” Nature Reviews Molecular Cell Biology; AOP, published online 29 Aug. 2013; doi:10.1038/nrm3645) the contents of which are incorporated herein by reference in its entirety.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present invention are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone (SEQ ID NO:153).

(SEQ ID NO: 153)
aaaaaaaaaa aaaaaaaaaa aaaaaaaaaa aaaaaaaaaa
aaaaaaaaaa aaaaaaaaaa aaaaaaaaaa aaaaaaaaaa
aaaaaaaaaa aaaaaaaaaa aaaaaaaaaa aaaaaaaaaa

### Start Codon Region

The invention also includes a polynucleotide that comprises both a start codon region and the polynucleotide described herein (e.g., a polynucleotide comprising a nucleotide sequence encoding a therapeutic payload or prophylactic payload, an effector molecule and/or a tether molecule). In some embodiments, the polynucleotides of the present invention can have regions that are analogous to or function like a start codon region.

In some embodiments, the translation of a polynucleotide can initiate on a codon that is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11; the contents of each of which are herein incorporated by reference in its entirety).

As a non-limiting example, the translation of a polynucleotide begins on the alternative start codon ACG. As another non-limiting example, polynucleotide translation begins on the alternative start codon CTG or CUG. As another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11; the contents of which are herein incorporated by reference in its entirety). Masking any of the nucleotides flanking a codon that initiates translation can be used to alter the position of translation initiation, translation efficiency, length and/or structure of a polynucleotide.

In some embodiments, a masking agent can be used near the start codon or alternative start codon to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11); the contents of which are herein incorporated by reference in its entirety).

In another embodiment, a masking agent can be used to mask a start codon of a polynucleotide to increase the likelihood that translation will initiate on an alternative start codon. In some embodiments, a masking agent can be used to mask a first start codon or alternative start codon to increase the chance that translation will initiate on a start codon or alternative start codon downstream to the masked start codon or alternative start codon.

In some embodiments, a start codon or alternative start codon can be located within a perfect complement for a miRNA binding site. The perfect complement of a miRNA binding site can help control the translation, length and/or structure of the polynucleotide similar to a masking agent. As a non-limiting example, the start codon or alternative start codon can be located in the middle of a perfect complement for a miRNA binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide can be removed from the polynucleotide sequence to have the translation of the polynucleotide begin on a codon that is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence to have translation initiate on a downstream start codon or alternative start codon. The polynucleotide sequence where the start codon was removed can further comprise at least one masking agent for the downstream start codon and/or alternative start codons to control or attempt to control the initiation of translation, the length of the polynucleotide and/or the structure of the polynucleotide.

### Stop Codon Region

The invention also includes a polynucleotide that comprises both a stop codon region and the polynucleotide described herein (e.g., a polynucleotide comprising a nucleotide sequence encoding a therapeutic payload or prophylactic payload, an effector molecule and/or a tether molecule). In some embodiments, the polynucleotides of the present invention can include at least two stop codons before the 3′ untranslated region (UTR). The stop codon can be selected from TGA, TAA and TAG in the case of DNA, or from UGA, UAA and UAG in the case of RNA. In some embodiments, the polynucleotides of the present invention include the stop codon TGA in the case or DNA, or the stop codon UGA in the case of RNA, and one additional stop codon. In a further embodiment the addition stop codon can be TAA or UAA. In another embodiment, the polynucleotides of the present invention include three consecutive stop codons, four stop codons, or more.

### Protease Cleavage Site or IRES

The invention also includes a polynucleotide that comprises a protease cleavage site or an internal ribosomal entry site. In some embodiments, the LNP compositions or systems of the present disclosure comprise: (a) a first polynucleotide (e.g., mRNA) comprising: (1) a sequence encoding a therapeutic payload or prophylactic payload, and (2) a binding element; and (b) a second polynucleotide (e.g., mRNA) comprising a sequence encoding: (1) an effector molecule, and (2) a polypeptide that recognizes the binding element (a tether molecule).

In some embodiments, the first polynucleotide and the second polynucleotide are disposed in the same polynucleotide.

In some embodiments, the first polynucleotide and the second polynucleotide are separated by a protease cleavage site, e.g., a T2A site or P2A site or E2A site, or TPE (P2A-T2A-E2A, or other known protease cleavage sites.

In some embodiments, the first polynucleotide and the second polynucleotide are separated by an IRES.

### Methods of Making Polynucleotides

The present disclosure also provides methods for making a polynucleotide disclosed herein or a complement thereof. In some aspects, a polynucleotide (e.g., an mRNA) disclosed herein encoding a therapeutic payload or prophylactic payload, an effector molecule and/or a tether molecule can be constructed using in vitro transcription.

In other aspects, a polynucleotide (e.g., an mRNA) disclosed herein encoding a therapeutic payload or prophylactic payload, an effector molecule and/or a tether molecule can be constructed by chemical synthesis using an oligonucleotide synthesizer. In other aspects, a polynucleotide (e.g., an mRNA) disclosed herein encoding a therapeutic payload or prophylactic payload, an effector molecule and/or a tether molecule is made by using a host cell. In certain aspects, a polynucleotide (e.g., an mRNA) disclosed herein encoding a therapeutic payload or prophylactic payload, an effector molecule and/or a tether molecule is made by one or more combination of the IVT, chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized nucleotide sequence (e.g., an mRNA) encoding a therapeutic payload or prophylactic payload, an effector molecule and/or a tether molecule. The resultant mRNAs can then be examined for their ability to produce protein and/or produce a therapeutic outcome.

Exemplary methods of making a polynucleotide disclosed herein include: in vitro transcription enzymatic synthesis and chemical synthesis which are disclosed in International PCT application WO 2017/201325, filed on 18 May 2017, the entire contents of which are hereby incorporated by reference.

### Purification

In other aspects, a polynucleotide (e.g., an mRNA) disclosed herein encoding a therapeutic payload or prophylactic payload, an effector molecule and/or a tether molecule can be purified. Purification of the polynucleotides (e.g., mRNA) encoding a therapeutic payload or prophylactic payload, an effector molecule and/or a tether molecule described herein can include, but is not limited to, polynucleotide clean-up, quality assurance and quality control. Clean-up can be performed by methods known in the arts such as, but not limited to, AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified polynucleotide” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

In some embodiments, purification of a polynucleotide (e.g., mRNA) encoding a therapeutic payload or prophylactic payload, an effector molecule and/or a tether molecule of the disclosure removes impurities that can reduce or remove an unwanted immune response, e.g., reducing cytokine activity.

In some embodiments, the polynucleotide (e.g., mRNA) encoding a therapeutic payload or prophylactic payload, an effector molecule and/or a tether molecule of the disclosure is purified prior to administration using column chromatography (e.g., strong anion exchange HPLC, weak anion exchange HPLC, reverse phase HPLC (RP-HPLC), and hydrophobic interaction HPLC (HIC-HPLC), or (LCMS)). In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide, which encodes a therapeutic payload or prophylactic payload, an effector molecule and/or a tether molecule disclosed herein increases expression of the therapeutic payload or prophylactic payload, an effector molecule and/or a tether molecule compared to polynucleotides encoding the therapeutic payload or prophylactic payload, an effector molecule and/or a tether molecule purified by a different purification method.

In some embodiments, a column chromatography (e.g., strong anion exchange HPLC, weak anion exchange HPLC, reverse phase HPLC (RP-HPLC), and hydrophobic interaction HPLC (HIC-HPLC), or (LCMS)) purified polynucleotide encodes a therapeutic payload or prophylactic payload, an effector molecule and/or a tether molecule. In some embodiments, the purified polynucleotide encodes a therapeutic payload or prophylactic payload, an effector molecule and/or a tether molecule.

In some embodiments, the purified polynucleotide is at least about 80% pure, at least about 85% pure, at least about 90% pure, at least about 95% pure, at least about 96% pure, at least about 97% pure, at least about 98% pure, at least about 99% pure, or about 100% pure.

A quality assurance and/or quality control check can be conducted using methods such as, but not limited to, gel electrophoresis, UV absorbance, or analytical HPLC.

In another embodiment, the polynucleotides can be sequenced by methods including, but not limited to reverse-transcriptase-PCR.

### Chemical Modifications of Polynucleotides

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise N1-methyl-pseudouridine (m1ψ), 1-ethyl-pseudouridine (e1ψ), 5-methoxy-uridine (mo5U), 5-methyl-cytidine (m5C), and/or pseudouridine (w). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises N1-methyl-pseudouridine (m1w) substitutions at one or more or all uridine positions of the nucleic acid.

In some embodiments, a RNA nucleic acid of the disclosure comprises N1-methyl-pseudouridine (m1w) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (w) substitutions at one or more or all uridine positions of the nucleic acid.

In some embodiments, a RNA nucleic acid of the disclosure comprises pseudouridine (w) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Pharmaceutical Compositions

The present disclosure provides pharmaceutical formulations comprising any of the systems, or LNP compositions disclosed herein, e.g., a system or an LNP composition comprising: (a) a first polynucleotide (e.g., mRNA) comprising: (1) a sequence encoding a therapeutic payload or prophylactic payload, and (2) a binding element; and (b) a second polynucleotide (e.g., mRNA) comprising a sequence encoding: (1) an effector molecule, and (2) a polypeptide that recognizes the binding element (a tether molecule).

In some embodiments of the disclosure, the polynucleotide are formulated in compositions and complexes in combination with one or more pharmaceutically acceptable excipients. Pharmaceutical compositions can optionally comprise one or more additional active substances, e.g. therapeutically and/or prophylactically active substances. Pharmaceutical compositions of the present disclosure can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005.

In some embodiments, compositions are administered to humans, human patients or subjects. For the purposes of the present disclosure, the phrase “active ingredient” generally refers to polynucleotides to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals.

In some embodiments, the polynucleotide of the present disclosure is formulated for subcutaneous, intravenous, intraperitoneal, intramuscular, intra-articular, intra-synovial, intrasternal, intrathecal, intrahepatic, intralesional, intracranial, intraventricular, oral, inhalation spray, pulmonary, topical, rectal, nasal, buccal, vaginal, or implanted reservoir intramuscular, subcutaneous, or intradermal delivery. In other embodiments, the polynucleotide is formulated for subcutaneous or intravenous delivery.

Formulations of the pharmaceutical compositions described herein can be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition can comprise between 0.1% and 100%, e.g., between 0.5% and 50%, between 1% and 30%, between 5% and 80%, or at least 80% (w/w) active ingredient.

### Formulations and Delivery

The polynucleotide comprising an mRNA of the disclosure can be formulated using one or more excipients.

The function of the one or more excipients is, e.g., to: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles, polymers, lipoplexes, core-shell nanoparticles, peptides, proteins, cells transfected with polynucleotides (e.g., for transplantation into a subject), hyaluronidase, nanoparticle mimics and combinations thereof. Accordingly, the formulations of the disclosure can include one or more excipients, each in an amount that together increases the stability of the polynucleotide, increases cell transfection by the polynucleotide, increases the expression of polynucleotides encoded protein, and/or alters the release profile of polynucleotide encoded proteins. Further, the polynucleotides of the present disclosure can be formulated using self-assembled nucleic acid nanoparticles.

Formulations of the pharmaceutical compositions described herein can be prepared by any method known or hereafter developed in the art of pharmacology. In general, such preparatory methods include the step of associating the active ingredient with an excipient and/or one or more other accessory ingredients.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered. For example, the composition can comprise between 0.1% and 99% (w/w) of the active ingredient. By way of example, the composition can comprise between 0.1% and 100%, e.g., between .5 and 50%, between 1-30%, between 5-80%, at least 80% (w/w) active ingredient.

In some embodiments, the formulations described herein contain at least one polynucleotide. As a non-limiting example, the formulations contain 1, 2, 3, 4 or 5 polynucleotides.

Pharmaceutical formulations can additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006). The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

In some embodiments, the particle size of the lipid nanoparticle is increased and/or decreased. The change in particle size can be able to help counter biological reaction such as, but not limited to, inflammation or can increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients can optionally be included in the pharmaceutical formulations of the disclosure.

In some embodiments, the polynucleotides is administered in or with, formulated in or delivered with nanostructures that can sequester molecules such as cholesterol. Non-limiting examples of these nanostructures and methods of making these nanostructures are described in US Patent Publication No. US20130195759. Exemplary structures of these nanostructures are shown in US Patent Publication No. US20130195759, and can include a core and a shell surrounding the core.

A polynucleotide comprising an mRNA of the disclosure can be delivered to a cell using any method known in the art. For example, the polynucleotide comprising an mRNA of the disclosure can be delivered to a cell by a lipid-based delivery, e.g., transfection, or by electroporation.

TABLE 6
Miscellaneous Sequences

| SEQ | Sequence | |
|---|---|---|
| ID NO | information | Sequence |
| 80 | EPO (aa) | MGVHECPAWLWLLLSLLSLPLGLPVLGAPPRLICDSRVLERYLLEA |
| | | KEAENITTGCAEHCSLNENITVPDTKVNFYAWKRMEVGQQAVEVWQG |
| | | LALLSEAVLRGQALLVNSSQPWEPLQLHVDKAVSGLRSLTTLLRALGA |
| | | QKEAISPPDAASAAPLRTITADTFRKLFRVYSNFLRGKLKLYTGEACRTG |
| | | DR |
| 81 | EPO (nt) | ATGGGAGTGCACGAGTGTCCCGCGTGGTTGTGGTTGCTGCTGTC |
| | | GCTCTTGAGCCTCCCACTGGGACTGCCTGTGCTGGGGGCACCACCCA |
| | | GATTGATCTGCGACTCACGGGTACTTGAGAGGTACCTTCTTGAAGCC |
| | | AAAGAAGCCGAAAACATCACAACCGGATGCGCCGAGCACTGCTCCC |
| | | TCAATGAGAACATTACTGTACCGGATACAAAGGTCAATTTCTATGCA |
| | | TGGAAGAGAATGGAAGTAGGACAGCAGGCCGTCGAAGTGTGGCAG |
| | | GGGCTCGCGCTTTTGTCGGAGGCGGTGTTGCGGGGTCAGGCCCTCCT |
| | | CGTCAACTCATCACAGCCGTGGGAGCCCCTCCAACTTCATGTCGATA |
| | | AAGCGGTGTCGGGGCTCCGCAGCTTGACGACGTTGCTTCGGGCTCTG |
| | | GGCGCACAAAAGGAGGCTATTTCGCCGCCTGACGCGGCCTCCGCGG |
| | | CACCCCTCCGAACGATCACCGCGGACACGTTTAGGAAGCTTTTTAGA |
| | | GTGTACAGCAATTTCCTCCGCGGAAAGCTGAAATTGTATACTGGTGA |
| | | AGCGTGTAGGACAGGGGATCGC |
| 82 | deg-GFP | MVSKGEELFTGVVPILVELDGDVNGHKFSVSGEGEGDATYGKLTL |
| | target (aa) | KFICTTGKLPVPWPTLVTTLTYGVQCFSRYPDHMKQHDFFKSAMPEGY |
| | | VQERTIFFKDDGNYKTRAEVKFEGDTLVNRIELKGIDFKEDGNILGHKL |
| | | EYNYNSHNVYIMADKQKNGIKVNFKIRHNIEDGSVQLADHYQQNTPIG |
| | | DGPVLLPDNHYLSTQSALSKDPNEKRDHMVLLEFVTAAGITLGMDELY |
| | | KRSRDISHGFPPAVAAQDDGTLPMSCAQESGMDRHPAACASARINV |
| 83 | deg-GFP | ATGGTGTCCAAGGGTGAGGAATTGTTTACCGGGGTGGTGCCTAT |
| | target (nt) | TCTCGTCGAACTTGACGGGGATGTGAATGGACACAAGTTTTCGGTAT |
| | | CCGGAGAAGGAGAGGGTGACGCCACATACGGAAAGCTTACACTCAA |
| | | ATTCATCTGTACGACGGGGAAACTGCCCGTACCCTGGCCTACGCTCG |
| | | TAACCACGCTGACTTATGGAGTGCAGTGCTTTAGCAGATACCCCGAC |
| | | CATATGAAGCAGCACGACTTCTTCAAGTCGGCGATGCCCGAGGGGT |
| | | ACGTGCAAGAGAGGACCATTTTCTTCAAAGACGATGGCAATTACAA |
| | | AACACGCGCAGAAGTCAAGTTTGAGGGCGATACTCTGGTCAATCGG |
| | | ATCGAATTGAAGGGAATCGATTTCAAAGAAGATGGAAACATCCTTG |
| | | GCCATAAGCTCGAGTACAACTATAACTCGCATAATGTCTATATCATG |
| | | GCTGACAAGCAGAAAAACGGTATCAAAGTCAACTTTAAGATCCGAC |
| | | ACAATATTGAGGACGGTTCGGTGCAGCTTGCGGACCACTATCAACA |
| | | GAATACGCCGATTGGGGATGGTCCGGTCCTTTTGCCGGATAACCATT |
| | | ATCTCTCAACCCAGTCAGCCCTGAGCAAAGATCCAAACGAGAAGAG |
| | | GGACCACATGGTCTTGCTCGAATTCGTGACAGCGGCAGGGATCACTC |
| | | TGGGAATGGACGAGTTGTACAAGAGATCTCGAGATATCAGCCATGG |
| | | CTTCCCGCCGGCGGTGGCGGCGCAGGATGATGGCACGCTGCCCATGT |
| | | CTTGTGCCCAGGAGAGCGGGATGGACCGTCACCCTGCAGCCTGTGCT |
| | | TCTGCTAGGATCAATGTG |
| 84 | polyA tail_100 | AAAAAAAAAAAAAAAAAAAAAAAAAAAAAAAAAAAAAAAAAA |
| | (nt) | AAAAAAAAAAAAAAAAAAAAAAAAAAAAAAAAAAAAAAAAAAAA |
| | | AAAAAAAAAAAAAA |
| 85 | Luc target (aa) | MEDAKNIKKGPAPFYPLEDGTAGEQLHKAMKRYALVPGTIAFTDA |
| | | HIEVDITYAEYFEMSVRLAEAMKRYGLNTNHRIVVCSENSLQFFMPVLG |
| | | ALFIGVAVAPANDIYNERELLNSMGISQPTVVFVSKKGLQKILNVQKKL |
| | | PIIQKIIIMDSKTDYQGFQSMYTFVTSHLPPGFNEYDFVPESFDRDKTIALI |
| | | MNSSGSTGLPKGVALPHRTACVRFSHARDPIFGNQIIPDTAILSVVPFHH |
| | | GFGMFTTLGYLICGFRVVLMYRFEEELFLRSLQDYKIQSALLVPTLFSFF |
| | | AKSTLIDKYDLSNLHEIASGGAPLSKEVGEAVAKRFHLPGIRQGYGLTE |
| | | TTSAILITPEGDDKPGAVGKVVPFFEAKVVDLDTGKTLGVNQRGELCVR |
| | | GPMIMSGYVNNPEATNALIDKDGWLHSGDIAYWDEDEHFFIVDRLKSLI |
| | | KYKGYQVAPAELESILLQHPNIFDAGVAGLPDDDAGELPAAVVVLEHG |
| | | KTMTEKEIVDYVASQVTTAKKLRGGVVFVDEVPKGLTGKLDARKIREI |
| | | LIKAKKGGKIAV |
| 86 | Luc target (nt) | ATGGAAGATGCGAAGAACATCAAGAAGGGACCTGCCCCGTTTTA |
| | | CCCTTTGGAGGACGGTACAGCAGGAGAACAGCTCCACAAGGCGATG |
| | | AAACGCTACGCCCTGGTCCCCGGAACGATTGCGTTTACCGATGCACA |
| | | TATTGAGGTAGACATCACATACGCAGAATACTTCGAAATGTCGGTGA |
| | | GGCTGGCGGAAGCGATGAAGAGATATGGTCTTAACACTAATCACCG |
| | | CATCGTGGTGTGTTCGGAGAACTCATTGCAGTTTTTCATGCCGGTCCT |
| | | TGGAGCACTTTTCATCGGGGTCGCAGTCGCGCCAGCGAACGACATCT |
| | | ACAATGAGCGGGAACTCTTGAATAGCATGGGAATCTCCCAGCCGAC |
| | | GGTCGTGTTTGTCTCCAAAAAGGGGCTGCAGAAAATCCTCAACGTGC |
| | | AGAAGAAGCTCCCCATTATTCAAAAGATCATCATTATGGATAGCAA |
| | | GACAGATTACCAAGGGTTCCAGTCGATGTATACCTTTGTGACATCGC |
| | | ATTTGCCGCCAGGGTTTAACGAGTATGACTTCGTCCCCGAGTCATTT |
| | | GACAGAGATAAAACCATCGCGCTGATTATGAATTCCTCGGGTAGCA |
| | | CCGGTTTGCCAAAGGGGGTGGCGTTGCCCCACCGCACTGCTTGTGTG |
| | | CGGTTCTCGCACGCTAGGGATCCTATCTTTGGTAATCAGATCATTCC |
| | | CGACACAGCAATCCTGTCCGTGGTACCTTTTCATCACGGTTTTGGCA |
| | | TGTTCACGACTCTCGGCTATTTGATTTGCGGTTTCAGGGTCGTACTTA |
| | | TGTATCGGTTCGAGGAAGAACTGTTTTTGAGATCCTTGCAAGATTAC |
| | | AAGATCCAGTCGGCCCTCCTTGTGCCAACGCTTTTCTCATTCTTTGCG |
| | | AAATCGACACTTATTGATAAGTATGACCTTTCCAATCTGCATGAGAT |
| | | TGCCTCAGGGGGAGCGCCGCTTAGCAAGGAAGTCGGGGAGGCAGTG |
| | | GCCAAGCGCTTCCACCTTCCCGGAATTCGGCAGGGATACGGGCTCAC |
| | | GGAGACAACATCCGCGATCCTTATCACGCCCGAGGGTGACGATAAG |
| | | CCGGGAGCCGTCGGAAAAGTGGTCCCCTTCTTTGAAGCCAAGGTCGT |
| | | AGACCTCGACACGGGAAAAACCCTCGGAGTGAACCAGAGGGGCGA |
| | | GCTCTGCGTGAGAGGGCCGATGATCATGTCAGGTTACGTGAATAACC |
| | | CTGAAGCGACGAATGCGCTGATCGACAAGGATGGGTGGTTGCATTC |
| | | GGGAGACATTGCCTATTGGGATGAGGATGAGCACTTCTTTATCGTAG |
| | | ATCGACTTAAGAGCTTGATCAAATACAAAGGCTATCAGGTAGCGCCT |
| | | GCCGAGCTCGAGTCAATCCTGCTCCAGCACCCCAACATTTTCGACGC |
| | | CGGAGTGGCCGGGTTGCCCGATGACGACGCGGGTGAGCTGCCAGCG |
| | | GCCGTGGTAGTCCTCGAACATGGGAAAACAATGACCGAAAAGGAGA |
| | | TCGTGGACTACGTAGCATCACAAGTGACGACTGCGAAGAAACTGAG |
| | | GGGAGGGGTAGTCTTTGTGGACGAGGTCCCGAAAGGCTTGACTGGG |
| | | AAGCTTGACGCTCGCAAAATCCGGGAAATCCTGATTAAGGCAAAGA |
| | | AAGGCGGGAAAATCGCTGTC |
| 87 | TPE element | GSGEGRGSLLTCGDVEENPGPGSGATNFSLLKQAGDVEENPGPGSG |
| | (aa) | QCTNYALLKLAGDVESNPGP |
| 88 | TPE element | GGCTCTGGCGAAGGCCGGGGTAGCCTGCTGACCTGCGGCGACGT |
| | (nt) | GGAGGAGAACCCTGGACCTGGATCCGGCGCCACCAACTTCAGCCTG |
| | | CTCAAGCAGGCCGGAGACGTGGAAGAGAATCCAGGCCCAGGCAGCG |
| | | GTCAGTGCACCAACTACGCCCTGCTGAAGCTGGCCGGCGACGTAGA |
| | | GAGCAACCCCGGCCCC |
| 89 | T2A self- | (GSG)EGRGSLLTCGDVEENPGP |
| | cleaving peptide | |
| | (aa)* | |
| 90 | P2A self- | (GSG)ATNFSLLKQAGDVEENPGP |
| | cleaving peptide | |
| | (aa)* | |
| 91 | E2A self- | (GSG)QCTNYALLKLAGDVESNPGP |
| | cleaving peptide | |
| | (aa)* | |
| 92 | F2A slef- | (GSG)VKQTLNFDLLKLAGDVESNPGP |
| | cleaving peptide | |
| | (aa)* | |
| 93 | LUC_TPE_ | MEDAKNIKKGPAPFYPLEDGTAGEQLHKAMKRYALVPGTIAFTDA |
| | MBPmid2 (aa) | HIEVDITYAEYFEMSVRLAEAMKRYGLNTNHRIVVCSENSLQFFMPVLG |
| | | ALFIGVAVAPANDIYNERELLNSMGISQPTVVFVSKKGLQKILNVQKKL |
| | | PIIQKIIIMDSKTDYQGFQSMYTFVTSHLPPGFNEYDFVPESFDRDKTIALI |
| | | MNSSGSTGLPKGVALPHRTACVRFSHARDPIFGNQIIPDTAILSVVPFHH |
| | | GFGMFTTLGYLICGFRVVLMYRFEEELFLRSLQDYKIQSALLVPTLFSFF |
| | | AKSTLIDKYDLSNLHEIASGGAPLSKEVGEAVAKRFHLPGIRQGYGLTE |
| | | TTSAILITPEGDDKPGAVGKVVPFFEAKVVDLDTGKTLGVNQRGELCVR |
| | | GPMIMSGYVNNPEATNALIDKDGWLHSGDIAYWDEDEHFFIVDRLKSLI |
| | | KYKGYQVAPAELESILLQHPNIFDAGVAGLPDDDAGELPAAVVVLEHG |
| | | KTMTEKEIVDYVASQVTTAKKLRGGVVFVDEVPKGLTGKLDARKIREI |
| | | LIKAKKGGKIAVGSGEGRGSLLTCGDVEENPGPGSGATNFSLLKQAGD |
| | | VEENPGPGSGQCTNYALLKLAGDVESNPGPASNFTQFVLVDNGGTGDV |
| | | TVAPSNFANGIAEWISSNSRSQAYKVTCSVRQSSAQNRKYTIKVEVPKG |
| | | AWRSYLNMELTIPIFATNSDCELIVKAMQGLLKDGNPIPSAIAANSGIYG |
| | | GGGSDPTRLQGINCGPDFTPSFANLGRTTLSTRGPPRGGPGGELPRGPAG |
| | | LGPRRSQQGPRKEPRKIIATVLMTEDIKLNKAEKAWKPSSKRTAADKDR |
| | | GEEDADGSKTQDLFRRVRSIANKATPQMAQQLMKQVTQLAIDTEERLK |
| | | GVIDLIFEKAISEPNFSVAYANMCRCLMALKVPTTEKPTVTVNFRKLLL |
| | | NRCQKEFEKDKDDDEVFEKKQKEMDEAATAEERGRLKEELEEARDIAR |
| | | RRSLGNIKFIGELFKLKMLTEAIMHDCVVKLLKNHDEESLECLCRLLTTI |
| | | GKDLDFEKAKPRMDQYFNQMEKIIKEKKTSSRIRFMLQDVLDLRGSNW |
| | | VPRRGDQGPKTIDQIHKEAEMEEHREHIKVQQLMAKGSDKRRGGPPGP |
| | | PISRGLPLVDDGGWNTVPISKGSRPIDTSRLTKITKPGSIDSNNQLFAPGG |
| | | RLSWGKGSSGGSGAKPSDAASEAARPATSTLNRFSALQQAV |
| 94 | LUC_TPE_ | ATGGAGGACGCCAAGAACATCAAGAAGGGGCCCGCTCCCTTCTA |
| | MBPmid2 (nt) | CCCGCTGGAGGACGGCACCGCTGGCGAGCAGCTGCACAAGGCCATG |
| | | AAGAGGTACGCACTGGTGCCCGGCACAATCGCCTTCACCGACGCCC |
| | | ACATCGAGGTGGACATCACCTACGCCGAGTACTTCGAGATGAGCGT |
| | | GCGGCTGGCCGAAGCCATGAAGCGGTACGGCCTGAACACCAACCAC |
| | | CGCATCGTGGTGTGCTCCGAGAACTCCCTGCAGTTCTTCATGCCCGT |
| | | TCTGGGCGCCCTGTTCATTGGGGTGGCCGTGGCACCAGCCAACGACA |
| | | TCTACAACGAGAGGGAGCTGCTGAACAGCATGGGCATCAGCCAGCC |
| | | CACCGTGGTGTTCGTGTCCAAGAAGGGCCTGCAGAAGATCCTGAAC |
| | | GTGCAGAAGAAGCTGCCCATCATCCAGAAGATCATCATCATGGACA |
| | | GCAAGACCGACTACCAGGGCTTCCAGTCCATGTACACCTTCGTGACC |
| | | AGCCACCTGCCTCCCGGCTTCAACGAGTACGACTTCGTGCCCGAGTC |
| | | CTTCGACAGGGACAAGACCATCGCCCTGATCATGAACTCCAGCGGA |
| | | AGCACCGGCCTGCCCAAGGGTGTGGCTCTACCCCACCGGACCGCCTG |
| | | TGTACGGTTCAGCCACGCCCGTGACCCCATCTTCGGGAACCAGATCA |
| | | TCCCCGACACCGCCATCCTGAGCGTGGTGCCCTTCCACCACGGCTTC |
| | | GGCATGTTCACCACCCTGGGCTACCTGATCTGCGGCTTCCGGGTGGT |
| | | GCTGATGTACCGGTTCGAGGAGGAGCTGTTCCTGCGGAGCCTGCAG |
| | | GACTACAAGATCCAGTCCGCCCTGCTGGTGCCCACCCTGTTCTCCTT |
| | | CTTCGCCAAGTCCACCCTGATCGACAAGTACGACCTGAGCAACCTGC |
| | | ACGAGATTGCCTCTGGCGGCGCACCTCTGTCCAAGGAGGTCGGGGA |
| | | GGCCGTAGCCAAGCGGTTCCACCTGCCCGGCATCCGGCAAGGCTAC |
| | | GGCCTGACCGAGACCACCAGCGCCATCCTGATCACCCCTGAGGGCG |
| | | ACGACAAGCCCGGCGCAGTGGGGAAGGTGGTGCCATTCTTCGAGGC |
| | | CAAGGTGGTGGACCTGGACACCGGCAAGACCCTGGGCGTGAACCAG |
| | | CGGGGCGAGCTGTGCGTGCGGGGACCCATGATCATGTCCGGCTACG |
| | | TGAACAACCCCGAGGCCACCAACGCCCTGATTGACAAGGACGGGTG |
| | | GCTGCACAGCGGCGACATCGCCTACTGGGACGAGGACGAGCACTTC |
| | | TTCATCGTGGACCGCCTGAAGTCCCTGATCAAGTACAAGGGGTACCA |
| | | GGTGGCTCCCGCCGAGCTGGAGTCCATCCTGCTGCAGCACCCCAACA |
| | | TCTTTGACGCCGGCGTTGCCGGACTGCCTGACGACGACGCTGGGGAA |
| | | CTCCCCGCAGCTGTGGTGGTGCTGGAGCACGGCAAGACCATGACCG |
| | | AGAAGGAGATCGTGGACTACGTCGCCTCCCAGGTGACCACCGCCAA |
| | | GAAGTTACGCGGCGGCGTCGTGTTCGTGGACGAGGTGCCCAAGGGC |
| | | CTGACCGGCAAGCTGGACGCCCGCAAGATCCGGGAGATCCTGATCA |
| | | AGGCCAAGAAGGGCGGGAAGATCGCCGTGGGCTCTGGCGAAGGCCG |
| | | GGGTAGCCTGCTGACCTGCGGCGACGTGGAGGAGAACCCTGGACCT |
| | | GGATCCGGCGCCACCAACTTCAGCCTGCTCAAGCAGGCCGGAGACG |
| | | TGGAAGAGAATCCAGGCCCAGGCAGCGGTCAGTGCACCAACTACGC |
| | | CCTGCTGAAGCTGGCCGGCGACGTAGAGAGCAACCCCGGCCCCGCC |
| | | AGCAACTTCACCCAGTTCGTGCTGGTGGACAACGGCGGTACCGGAG |
| | | ACGTGACCGTGGCCCCTTCTAACTTCGCCAACGGCATCGCCGAGTGG |
| | | ATCAGCAGCAACAGCAGAAGCCAGGCCTACAAGGTGACCTGCAGCG |
| | | TGAGACAGAGCAGCGCCCAGAACAGAAAGTACACCATCAAGGTGGA |
| | | GGTGCCTAAGGGCGCCTGGAGAAGCTACCTGAACATGGAGCTGACC |
| | | ATCCCTATCTTCGCCACCAACAGCGACTGCGAGCTGATCGTGAAGGC |
| | | CATGCAGGGCCTGCTGAAGGACGGCAACCCTATCCCTAGCGCCATC |
| | | GCCGCCAATTCAGGCATCTACGGTGGCGGAGGCAGCGACCCTACCA |
| | | GACTGCAGGGCATCAACTGCGGCCCTGACTTCACTCCTTCTTTCGCA |
| | | AACCTGGGCAGAACCACCCTGAGCACCAGAGGCCCTCCTAGAGGTG |
| | | GTCCCGGCGGAGAACTCCCCAGGGGTCCTGCCGGCCTGGGCCCTAG |
| | | ACGCTCCCAGCAAGGTCCTAGAAAGGAGCCAAGGAAGATCATCGCC |
| | | ACCGTGCTGATGACCGAGGACATCAAGCTGAACAAAGCTGAGAAGG |
| | | CCTGGAAGCCTAGCAGCAAGAGAACCGCCGCCGACAAGGACAGAG |
| | | GCGAGGAGGACGCCGACGGATCCAAGACCCAGGACCTGTTCAGAAG |
| | | AGTGAGAAGCATCGCCAACAAGGCGACCCCTCAGATGGCGCAGCAG |
| | | CTGATGAAGCAGGTGACGCAGCTCGCCATCGACACCGAGGAGAGAC |
| | | TGAAGGGCGTGATCGACCTGATCTTTGAGAAGGCTATCTCAGAGCCT |
| | | AACTTCAGCGTGGCCTACGCCAACATGTGCCGTTGCCTGATGGCATT |
| | | GAAGGTGCCAACCACCGAGAAGCCTACTGTGACCGTCAATTTCCGTA |
| | | AACTGCTGCTGAACCGGTGCCAGAAAGAGTTCGAGAAGGATAAGGA |
| | | CGACGACGAGGTCTTCGAGAAGAAACAGAAAGAAATGGACGAGGC |
| | | CGCCACCGCAGAGGAAAGGGGCCGATTAAAGGAGGAGCTGGAGGA |
| | | GGCCAGAGACATCGCCAGACGGCGTTCTCTGGGCAACATCAAGTTC |
| | | ATAGGTGAGCTGTTCAAGCTAAAGATGCTCACCGAGGCCATAATGC |
| | | ACGACTGCGTGGTGAAGCTACTGAAGAACCACGACGAAGAAAGCCT |
| | | GGAGTGCCTGTGCAGACTGCTGACCACCATCGGCAAGGACCTGGAC |
| | | TTCGAGAAGGCAAAGCCTCGAATGGACCAGTACTTCAACCAGATGG |
| | | AGAAGATTATCAAGGAGAAGAAGACCAGCAGCAGAATCAGATTCAT |
| | | GCTGCAGGACGTACTGGACCTGCGCGGAAGCAACTGGGTGCCAAGG |
| | | AGAGGGGACCAAGGACCAAAGACCATCGACCAGATCCACAAGGAA |
| | | GCGGAGATGGAGGAGCACAGAGAGCACATAAAGGTGCAGCAGCTT |
| | | ATGGCCAAGGGCAGCGACAAGCGAAGAGGCGGCCCGCCCGGACCTC |
| | | CTATCAGCAGAGGCCTTCCTCTGGTAGACGACGGCGGCTGGAACAC |
| | | CGTGCCTATCTCTAAGGGCTCCAGACCTATCGACACTTCCCGTCTTA |
| | | CCAAGATCACCAAGCCAGGATCTATTGACAGCAACAACCAGCTGTT |
| | | CGCCCCAGGAGGAAGACTTAGCTGGGGCAAGGGAAGTTCCGGCGGA |
| | | TCCGGCGCCAAGCCTTCCGACGCCGCCAGCGAGGCTGCCAGACCTG |
| | | CCACCAGCACCTTGAACCGCTTTTCCGCTCTGCAGCAAGCTGTG |
| 95 | LUC-TPE- | MEDAKNIKKGPAPFYPLEDGTAGEQLHKAMKRYALVPGTIAFTDA |
| | MBPLACZ (aa) | HIEVDITYAEYFEMSVRLAEAMKRYGLNTNHRIVVCSENSLQFFMPVLG |
| | | ALFIGVAVAPANDIYNERELLNSMGISQPTVVFVSKKGLQKILNVQKKL |
| | | PIIQKIIIMDSKTDYQGFQSMYTFVTSHLPPGFNEYDFVPESFDRDKTIALI |
| | | MNSSGSTGLPKGVALPHRTACVRFSHARDPIFGNQIIPDTAILSVVPFHH |
| | | GFGMFTTLGYLICGFRVVLMYRFEEELFLRSLQDYKIQSALLVPTLFSFF |
| | | AKSTLIDKYDLSNLHEIASGGAPLSKEVGEAVAKRFHLPGIRQGYGLTE |
| | | TTSAILITPEGDDKPGAVGKVVPFFEAKVVDLDTGKTLGVNQRGELCVR |
| | | GPMIMSGYVNNPEATNALIDKDGWLHSGDIAYWDEDEHFFIVDRLKSLI |
| | | KYKGYQVAPAELESILLQHPNIFDAGVAGLPDDDAGELPAAVVVLEHG |
| | | KTMTEKEIVDYVASQVTTAKKLRGGVVFVDEVPKGLTGKLDARKIREI |
| | | LIKAKKGGKIAVGSGEGRGSLLTCGDVEENPGPGSGATNFSLLKQAGD |
| | | VEENPGPGSGQCTNYALLKLAGDVESNPGPASNFTQFVLVDNGGTGDV |
| | | TVAPSNFANGIAEWISSNSRSQAYKVTCSVRQSSAQNRKYTIKVEVPKG |
| | | AWRSYLNMELTIPIFATNSDCELIVKAMQGLLKDGNPIPSAIAANSGIYG |
| | | GGGSSFTLTNKNVIFVAGLGGIGLDTSKELLKRDPVVLQRRDWENPGVT |
| | | QLNRLAAHPPFASWRNSEEARTDRPSQQLRSLNGEWRFAWFPAPEAVP |
| | | ESWLECDLPEADTVVVPSNWQMHGYDAPIYTNVTYPITVNPPFVPTENP |
| | | TGCYSLTFNVDESWLQEGQTRIIFDGVNSAFHLWCNGRWVGYGQDSRL |
| | | PSEFDLSAFLRAGENRLAVMVLRWSDGSYLEDQDMWRMSGIFRDVSLL |
| | | HKPTTQISDFHVATRFNDDFSRAVLEAEVQMCGELRDYLRVTVSLWQG |
| | | ETQVASGTAPFGGEIIDERGGYADRVTLRLNVENPKLWSAEIPNLYRAV |
| | | VELHTADGTLIEAEACDVGFREVRIENGLLLLNGKPLLIRGVNRHEHHP |
| | | LHGQVMDEQTMVQDILLMKQNNFNAVRCSHYPNHPLWYTLCDRYGL |
| | | YVVDEANIETHGMVPMNRLTDDPRWLPAMSERVTRMVQRDRNHPSVII |
| | | WSLGNESGHGANHDALYRWIKSVDPSRPVQYEGGGADTTATDIICPMY |
| | | ARVDEDQPFPAVPKWSIKKWLSLPGETRPLILCEYAHAMGNSLGGFAK |
| | | YWQAFRQYPRLQGGFVWDWVDQSLIKYDENGNPWSAYGGDFGDTPN |
| | | DRQFCMNGLVFADRTPHPALTEAKHQQQFFQFRLSGQTIEVTSEYLFRH |
| | | SDNELLHWMVALDGKPLASGEVPLDVAPQGKQLIELPELPQPESAGQL |
| | | WLTVRVVQPNATAWSEAGHISAWQQWRLAENLSVTLPAASHAIPHLTT |
| | | SEMDFCIELGNKRWQFNRQSGFLSQMWIGDKKQLLTPLRDQFTRAPLD |
| | | NDIGVSEATRIDPNAWVERWKAAGHYQAEAALLQCTADTLADAVLITT |
| | | AHAWQHQGKTLFISRKTYRIDGSGQMAITVDVEVASDTPHPARIGLNC |
| | | QLAQVAERVNWLGLGPQENYPDRLTAACFDRWDLPLSDMYTPYVFPS |
| | | ENGLRCGTRELNYGPHQWRGDFQFNISRYSQQQLMETSHRHLLHAEEG |
| | | TWLNIDGFHMGIGGDDSWSPSVSAELQLSAGRYHYQLVWCQK |
| 96 | LUC-TPE- | ATGGAGGACGCCAAGAACATCAAGAAGGGGCCCGCTCCCTTCTA |
| | MBPLACZ (nt) | CCCGCTGGAGGACGGCACCGCTGGCGAGCAGCTGCACAAGGCCATG |
| | | AAGAGGTACGCACTGGTGCCCGGCACAATCGCCTTCACCGACGCCC |
| | | ACATCGAGGTGGACATCACCTACGCCGAGTACTTCGAGATGAGCGT |
| | | GCGGCTGGCCGAAGCCATGAAGCGGTACGGCCTGAACACCAACCAC |
| | | CGCATCGTGGTGTGCTCCGAGAACTCCCTGCAGTTCTTCATGCCCGT |
| | | TCTGGGCGCCCTGTTCATTGGGGTGGCCGTGGCACCAGCCAACGACA |
| | | TCTACAACGAGAGGGAGCTGCTGAACAGCATGGGCATCAGCCAGCC |
| | | CACCGTGGTGTTCGTGTCCAAGAAGGGCCTGCAGAAGATCCTGAAC |
| | | GTGCAGAAGAAGCTGCCCATCATCCAGAAGATCATCATCATGGACA |
| | | GCAAGACCGACTACCAGGGCTTCCAGTCCATGTACACCTTCGTGACC |
| | | AGCCACCTGCCTCCCGGCTTCAACGAGTACGACTTCGTGCCCGAGTC |
| | | CTTCGACAGGGACAAGACCATCGCCCTGATCATGAACTCCAGCGGA |
| | | AGCACCGGCCTGCCCAAGGGTGTGGCTCTACCCCACCGGACCGCCTG |
| | | TGTACGGTTCAGCCACGCCCGTGACCCCATCTTCGGGAACCAGATCA |
| | | TCCCCGACACCGCCATCCTGAGCGTGGTGCCCTTCCACCACGGCTTC |
| | | GGCATGTTCACCACCCTGGGCTACCTGATCTGCGGCTTCCGGGTGGT |
| | | GCTGATGTACCGGTTCGAGGAGGAGCTGTTCCTGCGGAGCCTGCAG |
| | | GACTACAAGATCCAGTCCGCCCTGCTGGTGCCCACCCTGTTCTCCTT |
| | | CTTCGCCAAGTCCACCCTGATCGACAAGTACGACCTGAGCAACCTGC |
| | | ACGAGATTGCCTCTGGCGGCGCACCTCTGTCCAAGGAGGTCGGGGA |
| | | GGCCGTAGCCAAGCGGTTCCACCTGCCCGGCATCCGGCAAGGCTAC |
| | | GGCCTGACCGAGACCACCAGCGCCATCCTGATCACCCCTGAGGGCG |
| | | ACGACAAGCCCGGCGCAGTGGGGAAGGTGGTGCCATTCTTCGAGGC |
| | | CAAGGTGGTGGACCTGGACACCGGCAAGACCCTGGGCGTGAACCAG |
| | | CGGGGCGAGCTGTGCGTGCGGGGACCCATGATCATGTCCGGCTACG |
| | | TGAACAACCCCGAGGCCACCAACGCCCTGATTGACAAGGACGGGTG |
| | | GCTGCACAGCGGCGACATCGCCTACTGGGACGAGGACGAGCACTTC |
| | | TTCATCGTGGACCGCCTGAAGTCCCTGATCAAGTACAAGGGGTACCA |
| | | GGTGGCTCCCGCCGAGCTGGAGTCCATCCTGCTGCAGCACCCCAACA |
| | | TCTTTGACGCCGGCGTTGCCGGACTGCCTGACGACGACGCTGGGGAA |
| | | CTCCCCGCAGCTGTGGTGGTGCTGGAGCACGGCAAGACCATGACCG |
| | | AGAAGGAGATCGTGGACTACGTCGCCTCCCAGGTGACCACCGCCAA |
| | | GAAGTTACGCGGCGGCGTCGTGTTCGTGGACGAGGTGCCCAAGGGC |
| | | CTGACCGGCAAGCTGGACGCCCGCAAGATCCGGGAGATCCTGATCA |
| | | AGGCCAAGAAGGGCGGGAAGATCGCCGTGGGCTCTGGCGAAGGCCG |
| | | GGGTAGCCTGCTGACCTGCGGCGACGTGGAGGAGAACCCTGGACCT |
| | | GGATCCGGCGCCACCAACTTCAGCCTGCTCAAGCAGGCCGGAGACG |
| | | TGGAAGAGAATCCAGGCCCAGGCAGCGGTCAGTGCACCAACTACGC |
| | | CCTGCTGAAGCTGGCCGGCGACGTAGAGAGCAACCCCGGCCCCGCC |
| | | AGCAACTTCACCCAGTTCGTGCTGGTGGACAACGGCGGTACCGGAG |
| | | ACGTGACCGTGGCCCCTTCTAACTTCGCCAACGGCATCGCCGAGTGG |
| | | ATCAGCAGCAACAGCAGAAGCCAGGCCTACAAGGTGACCTGCAGCG |
| | | TGAGACAGAGCAGCGCCCAGAACAGAAAGTACACCATCAAGGTGGA |
| | | GGTGCCTAAGGGCGCCTGGAGAAGCTACCTGAACATGGAGCTGACC |
| | | ATCCCTATCTTCGCCACCAACAGCGACTGCGAGCTGATCGTGAAGGC |
| | | CATGCAGGGCCTGCTGAAGGACGGCAACCCTATCCCTAGCGCCATC |
| | | GCCGCCAATTCAGGCATCTACGGAGGCGGTGGAAGCTCGTTCACGCT |
| | | AACGAACAAGAACGTCATCTTCGTAGCGGGACTTGGCGGTATCGGC |
| | | CTAGACACGTCGAAGGAACTACTAAAGCGTGACCCGGTAGTCCTCC |
| | | AACGTCGCGATTGGGAGAACCCGGGCGTAACGCAACTAAACCGTCT |
| | | TGCGGCGCACCCGCCGTTTGCGTCGTGGCGTAACTCGGAGGAGGCG |
| | | CGAACGGATCGTCCGTCGCAACAACTACGTTCGCTCAACGGGGAGT |
| | | GGCGCTTCGCGTGGTTCCCGGCGCCGGAGGCGGTACCGGAGTCGTG |
| | | GCTCGAGTGCGATCTACCGGAGGCGGACACGGTCGTCGTACCGTCG |
| | | AACTGGCAAATGCACGGTTACGACGCGCCGATATACACGAACGTCA |
| | | CGTACCCGATAACGGTAAACCCGCCGTTCGTCCCGACGGAGAACCC |
| | | GACGGGGTGCTACTCGCTAACGTTCAACGTTGACGAGTCGTGGTTGC |
| | | AAGAGGGTCAAACGCGTATCATATTCGACGGTGTAAACTCGGCGTTC |
| | | CACCTGTGGTGCAACGGGCGCTGGGTAGGGTACGGCCAAGACTCGC |
| | | GTCTACCGTCGGAGTTCGACCTATCGGCGTTCCTACGAGCGGGTGAG |
| | | AACCGGCTAGCGGTCATGGTCCTACGTTGGTCGGACGGTTCGTACCT |
| | | CGAGGACCAAGACATGTGGCGAATGTCGGGTATCTTCCGCGACGTA |
| | | TCGCTCCTACACAAGCCGACGACGCAAATCTCGGACTTCCACGTCGC |
| | | GACGCGTTTCAACGACGATTTCTCGCGGGCAGTCCTAGAGGCGGAG |
| | | GTCCAAATGTGCGGGGAGCTACGTGACTACCTCCGTGTCACGGTATC |
| | | GCTCTGGCAAGGTGAGACGCAAGTAGCGTCGGGTACGGCGCCGTTC |
| | | GGCGGTGAGATCATCGACGAGCGTGGTGGGTACGCGGACCGTGTAA |
| | | CGCTACGTCTAAACGTCGAGAACCCGAAGCTCTGGTCGGCGGAGAT |
| | | CCCGAACCTATACCGTGCGGTCGTCGAGCTACATACGGCGGACGGG |
| | | ACGCTAATAGAGGCGGAAGCGTGCGACGTCGGGTTTCGAGAGGTTC |
| | | GTATAGAGAACGGGCTGCTACTTCTAAACGGGAAGCCGTTGCTCATA |
| | | CGTGGTGTCAACCGTCACGAGCACCACCCGCTACACGGTCAAGTAAT |
| | | GGACGAGCAAACGATGGTACAAGACATCCTACTAATGAAGCAGAAC |
| | | AACTTCAACGCGGTACGCTGTTCGCATTACCCGAACCATCCGTTGTG |
| | | GTACACGCTTTGCGACCGATACGGTCTATACGTCGTAGACGAGGCGA |
| | | ACATAGAGACGCACGGGATGGTACCGATGAATCGCCTAACGGACGA |
| | | CCCGCGTTGGCTACCGGCGATGTCGGAGCGAGTCACGCGTATGGTCC |
| | | AACGGGACCGTAACCACCCGTCGGTAATAATCTGGTCGCTAGGCAA |
| | | CGAATCGGGGCACGGGGCGAACCACGACGCGCTATACCGTTGGATC |
| | | AAGTCGGTAGACCCGTCGCGTCCGGTACAATACGAAGGTGGCGGTG |
| | | CGGACACGACGGCGACGGACATCATCTGCCCGATGTACGCGCGCGT |
| | | CGACGAAGACCAACCGTTCCCGGCGGTACCGAAGTGGTCGATCAAG |
| | | AAGTGGCTCTCGTTGCCGGGTGAAACGCGTCCGTTGATACTTTGCGA |
| | | GTACGCGCACGCGATGGGCAACTCGTTGGGTGGGTTCGCGAAGTAC |
| | | TGGCAGGCGTTCCGTCAATACCCGCGTCTACAGGGTGGGTTCGTCTG |
| | | GGACTGGGTAGACCAATCGCTAATCAAGTACGACGAGAACGGCAAC |
| | | CCGTGGTCGGCGTACGGTGGGGACTTCGGGGACACGCCGAACGACC |
| | | GCCAATTCTGTATGAACGGCCTAGTCTTCGCGGACCGAACGCCGCAC |
| | | CCGGCGTTGACGGAGGCGAAGCATCAACAACAATTCTTCCAATTCCG |
| | | TCTATCGGGGCAAACGATCGAGGTAACGTCGGAGTACTTGTTCCGGC |
| | | ACTCGGACAACGAGCTACTACACTGGATGGTAGCACTAGACGGCAA |
| | | GCCGCTAGCGTCGGGAGAAGTCCCTTTGGACGTCGCGCCGCAAGGT |
| | | AAGCAACTAATCGAGCTACCGGAGCTACCGCAACCGGAGTCGGCGG |
| | | GTCAACTGTGGTTGACGGTCCGTGTCGTTCAACCGAACGCGACGGCG |
| | | TGGTCGGAGGCGGGTCACATCTCGGCGTGGCAGCAGTGGCGTCTAG |
| | | CGGAGAACCTCTCGGTCACGCTACCGGCGGCGTCGCACGCGATACC |
| | | GCATCTAACGACGTCGGAGATGGACTTCTGCATCGAGTTGGGGAAC |
| | | AAGAGGTGGCAGTTCAACCGTCAATCGGGATTCCTATCGCAAATGTG |
| | | GATAGGTGACAAGAAGCAACTACTAACGCCGCTACGTGATCAGTTC |
| | | ACGCGTGCTCCGCTAGACAACGACATAGGTGTTTCGGAGGCGACGC |
| | | GTATAGACCCGAACGCGTGGGTGGAGCGGTGGAAGGCGGCGGGGCA |
| | | CTACCAAGCGGAGGCGGCGCTACTACAGTGCACGGCGGACACGCTA |
| | | GCGGACGCGGTATTGATCACGACGGCGCACGCGTGGCAACACCAGG |
| | | GGAAGACGCTATTCATCTCGCGTAAGACGTACCGTATCGACGGTTCG |
| | | GGCCAAATGGCGATCACGGTCGACGTAGAGGTAGCGTCGGACACGC |
| | | CGCATCCGGCGCGCATCGGTCTAAACTGCCAACTAGCGCAAGTAGC |
| | | GGAGCGTGTAAACTGGCTAGGGCTAGGGCCGCAAGAGAACTATCCG |
| | | GACCGCCTAACGGCGGCGTGCTTCGACCGTTGGGACCTACCGCTTTC |
| | | GGACATGTATACCCCGTACGTCTTCCCGTCGGAGAACGGGTTGAGGT |
| | | GCGGGACGCGCGAGCTAAACTACGGGCCGCACCAGTGGCGAGGGGA |
| | | CTTCCAATTCAACATATCGCGTTACTCGCAACAACAACTAATGGAGA |
| | | CGTCGCACCGTCACCTACTACACGCGGAGGAGGGGACGTGGCTAAA |
| | | CATCGACGGGTTCCACATGGGCATAGGTGGGGACGACTCGTGGTCG |
| | | CCGTCGGTCTCGGCGGAGCTCCAACTCTCGGCGGGTCGTTACCATTA |
| | | CCAACTAGTTTGGTGCCAGAAG |
| 97 | LacZ (aa) | MSFTLTNKNVIFVAGLGGIGLDTSKELLKRDPVVLQRRDWENPGVT |
| | | QLNRLAAHPPFASWRNSEEARTDRPSQQLRSLNGEWRFAWFPAPEAVP |
| | | ESWLECDLPEADTVVVPSNWQMHGYDAPIYTNVTYPITVNPPFVPTENP |
| | | TGCYSLTFNVDESWLQEGQTRIIFDGVNSAFHLWCNGRWVGYGQDSRL |
| | | PSEFDLSAFLRAGENRLAVMVLRWSDGSYLEDQDMWRMSGIFRDVSLL |
| | | HKPTTQISDFHVATRFNDDFSRAVLEAEVQMCGELRDYLRVTVSLWQG |
| | | ETQVASGTAPFGGEIIDERGGYADRVTLRLNVENPKLWSAEIPNLYRAV |
| | | VELHTADGTLIEAEACDVGFREVRIENGLLLLNGKPLLIRGVNRHEHHP |
| | | LHGQVMDEQTMVQDILLMKQNNFNAVRCSHYPNHPLWYTLCDRYGL |
| | | YVVDEANIETHGMVPMNRLTDDPRWLPAMSERVTRMVQRDRNHPSVII |
| | | WSLGNESGHGANHDALYRWIKSVDPSRPVQYEGGGADTTATDIICPMY |
| | | ARVDEDQPFPAVPKWSIKKWLSLPGETRPLILCEYAHAMGNSLGGFAK |
| | | YWQAFRQYPRLQGGFVWDWVDQSLIKYDENGNPWSAYGGDFGDTPN |
| | | DRQFCMNGLVFADRTPHPALTEAKHQQQFFQFRLSGQTIEVTSEYLFRH |
| | | SDNELLHWMVALDGKPLASGEVPLDVAPQGKQLIELPELPQPESAGQL |
| | | WLTVRVVQPNATAWSEAGHISAWQQWRLAENLSVTLPAASHAIPHLTT |
| | | SEMDFCIELGNKRWQFNRQSGFLSQMWIGDKKQLLTPLRDQFTRAPLD |
| | | NDIGVSEATRIDPNAWVERWKAAGHYQAEAALLQCTADTLADAVLITT |
| | | AHAWQHQGKTLFISRKTYRIDGSGQMAITVDVEVASDTPHPARIGLNC |
| | | QLAQVAERVNWLGLGPQENYPDRLTAACFDRWDLPLSDMYTPYVFPS |
| | | ENGLRCGTRELNYGPHQWRGDFQFNISRYSQQQLMETSHRHLLHAEEG |
| | | TWLNIDGFHMGIGGDDSWSPSVSAELQLSAGRYHYQLVWCQK |
| 98 | LacZ (nt) | ATGAGCTTCACCCTGACCAACAAGAACGTGATCTTCGTGGCCGG |
| | | CCTGGGCGGCATCGGCCTGGACACCAGCAAGGAGCTGCTGAAGCGG |
| | | GACCCCGTGGTGCTGCAGCGCCGGGACTGGGAGAACCCCGGCGTGA |
| | | CCCAGCTGAACAGGCTGGCCGCCCATCCTCCGTTCGCCTCCTGGCGG |
| | | AACTCCGAGGAGGCCCGGACCGACCGGCCCTCCCAGCAGCTGCGGT |
| | | CTCTGAACGGCGAGTGGAGGTTCGCCTGGTTCCCCGCCCCAGAGGCC |
| | | GTGCCTGAGAGCTGGCTGGAGTGCGACCTGCCTGAAGCCGACACCG |
| | | TGGTGGTGCCTAGCAACTGGCAGATGCACGGCTACGACGCACCCAT |
| | | CTACACCAACGTGACCTACCCTATCACCGTGAATCCGCCTTTCGTGC |
| | | CCACCGAGAACCCCACCGGCTGCTACAGCCTGACCTTCAACGTGGAC |
| | | GAGAGTTGGCTGCAGGAGGGCCAGACCCGCATCATCTTCGACGGCG |
| | | TGAACAGCGCCTTCCACCTGTGGTGCAACGGCCGGTGGGTGGGCTAC |
| | | GGCCAGGACTCCCGGCTGCCCTCCGAGTTTGACCTGTCCGCCTTCCT |
| | | GCGCGCCGGCGAGAACCGGCTGGCCGTGATGGTGCTGAGGTGGAGC |
| | | GACGGCAGCTACCTGGAGGACCAGGACATGTGGCGCATGTCCGGAA |
| | | TCTTCCGGGACGTGAGCCTGCTGCACAAGCCCACCACCCAGATCAGC |
| | | GACTTCCACGTGGCCACCCGGTTCAACGACGACTTCAGCCGGGCCGT |
| | | GCTGGAGGCCGAGGTGCAGATGTGCGGCGAGCTGCGGGACTACCTG |
| | | CGCGTGACCGTGAGCCTGTGGCAGGGCGAGACCCAGGTGGCTTCCG |
| | | GCACCGCCCCATTCGGCGGCGAGATCATCGACGAGCGGGGCGGCTA |
| | | CGCCGACCGGGTGACCCTGCGGCTGAACGTGGAGAACCCCAAGCTG |
| | | TGGAGCGCCGAGATCCCCAACCTGTACCGGGCCGTGGTGGAGCTGC |
| | | ACACCGCCGACGGCACCCTGATCGAGGCCGAGGCCTGCGACGTGGG |
| | | CTTCCGGGAGGTGAGGATCGAGAACGGGCTGCTGCTGCTGAACGGG |
| | | AAGCCACTGCTGATCAGGGGCGTGAACCGCCACGAGCACCATCCCC |
| | | TGCACGGCCAGGTGATGGACGAGCAGACCATGGTGCAGGACATCCT |
| | | GCTGATGAAGCAGAACAACTTCAACGCCGTGAGGTGCTCCCACTAC |
| | | CCCAACCATCCCCTGTGGTACACCCTGTGCGACCGGTACGGCCTGTA |
| | | CGTGGTGGACGAGGCCAACATCGAGACCCACGGGATGGTCCCAATG |
| | | AACCGGCTGACTGACGACCCTCGCTGGCTGCCCGCCATGAGCGAGC |
| | | GGGTGACCAGGATGGTGCAGCGCGACCGGAACCACCCCAGCGTGAT |
| | | CATCTGGAGCCTGGGGAACGAGTCCGGCCACGGCGCCAACCACGAC |
| | | GCCCTGTACCGCTGGATCAAGAGCGTGGACCCCTCCCGCCCCGTGCA |
| | | GTACGAGGGCGGCGGCGCCGACACCACCGCCACCGACATCATCTGC |
| | | CCCATGTACGCCCGGGTGGACGAGGACCAGCCCTTCCCTGCCGTGCC |
| | | CAAGTGGAGCATCAAGAAGTGGCTGAGCTTGCCCGGCGAGACCAGA |
| | | CCGCTGATCCTGTGCGAGTACGCCCACGCCATGGGCAACTCCCTGGG |
| | | CGGCTTCGCCAAGTACTGGCAGGCCTTCCGGCAGTACCCGCGGCTGC |
| | | AGGGCGGCTTCGTGTGGGACTGGGTGGACCAGTCCCTGATCAAGTA |
| | | CGACGAGAACGGCAACCCCTGGAGCGCCTACGGCGGGGACTTCGGG |
| | | GACACACCCAACGACCGCCAGTTCTGCATGAACGGCCTGGTGTTCGC |
| | | CGACCGCACCCCGCACCCTGCCCTGACCGAGGCCAAGCACCAGCAG |
| | | CAGTTCTTCCAGTTCCGGCTGAGCGGGCAGACCATCGAGGTGACCAG |
| | | CGAGTACCTGTTCCGGCACAGCGACAACGAGCTGCTGCACTGGATG |
| | | GTGGCCCTGGACGGCAAGCCCCTGGCCAGCGGCGAGGTGCCTCTGG |
| | | ACGTGGCTCCCCAGGGCAAGCAGCTGATCGAGCTGCCCGAGCTGCC |
| | | CCAGCCCGAGAGCGCCGGCCAGCTGTGGCTGACCGTGAGGGTGGTG |
| | | CAGCCCAACGCCACCGCCTGGAGCGAGGCCGGCCACATCAGCGCCT |
| | | GGCAACAGTGGCGGCTGGCCGAGAACCTGAGCGTGACCCTGCCCGC |
| | | CGCCAGCCACGCCATCCCTCACCTGACCACCTCCGAGATGGACTTCT |
| | | GCATCGAGCTGGGCAACAAGCGGTGGCAGTTCAACCGGCAGTCCGG |
| | | CTTCCTGAGCCAGATGTGGATCGGCGACAAGAAGCAGCTGCTGACC |
| | | CCACTGCGGGACCAGTTCACCAGGGCCCCACTGGACAACGACATCG |
| | | GCGTGAGCGAGGCCACCAGAATCGACCCCAACGCCTGGGTGGAGCG |
| | | GTGGAAGGCCGCCGGCCACTACCAGGCCGAGGCCGCCCTGCTGCAG |
| | | TGCACCGCCGACACCCTGGCCGACGCCGTGCTGATCACCACCGCCCA |
| | | CGCCTGGCAGCACCAGGGGAAGACCCTGTTCATCAGCCGGAAGACC |
| | | TACCGGATCGACGGGTCCGGCCAGATGGCCATCACCGTGGACGTGG |
| | | AGGTGGCCTCCGACACCCCTCACCCCGCCCGGATCGGCCTGAACTGT |
| | | CAGCTGGCCCAGGTGGCCGAGCGGGTGAACTGGCTGGGGCTGGGCC |
| | | CACAGGAGAACTACCCCGACCGGCTGACCGCCGCCTGCTTCGACCG |
| | | GTGGGACCTGCCCCTGAGCGACATGTACACCCCATACGTGTTCCCCT |
| | | CTGAGAACGGCCTGCGGTGCGGCACCCGGGAGCTGAACTACGGGCC |
| | | CCACCAGTGGCGCGGCGACTTCCAGTTCAACATCAGCCGGTACAGCC |
| | | AGCAGCAGCTGATGGAGACCAGCCACCGGCACCTGCTGCACGCCGA |
| | | GGAGGGCACCTGGCTGAACATCGACGGCTTCCACATGGGCATCGGC |
| | | GGCGACGACAGCTGGTCCCCATCCGTGAGCGCCGAGCTGCAGCTGA |
| | | GCGCCGGCCGGTACCACTACCAGCTGGTGTGGTGCCAGAAG |
| 153 | Poly A tail_120 | AAAAAAAAAAAAAAAAAAAAAAAAAAAAAAAAAAAAAAAAAA |
| | (nt) | AAAAAAAAAAAAAAAAAAAAAAAAAAAAAAAAAAAAAAAAAAAA |
| | | AAAAAAAAAAAAAAAAAAAAAAAAAAAAAAAAAA |

*(GSG) is an optional element

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1

### Production of LNP Compositions

### A. Production of Nanoparticle Compositions

In order to investigate safe and efficacious nanoparticle compositions for use in the delivery of therapeutic and/or prophylactics to cells, a range of formulations are prepared and tested. Specifically, the particular elements and ratios thereof in the lipid component of nanoparticle compositions are optimized.

Nanoparticles can be made with mixing processes such as microfluidics and T-junction mixing of two fluid streams, one of which contains the therapeutic and/or prophylactic and the other has the lipid components.

Lipid compositions are prepared by combining a lipid according to Formulae (I), (IA), (IB), (II), (IIa), (IIb), (IIc), (IId), (IIe), (IIf), (IIg), (III), (IIIa1), (IIIa2), (IIIa3), (IIIa4), (IIIa5), (IIIa6), (IIIa7), or (IIIa8) or a non-cationic helper lipid (such as DOPE, or DSPC obtainable from Avanti Polar Lipids, Alabaster, AL), a PEG lipid (such as 1,2 dimyristoyl sn glycerol methoxypolyethylene glycol, also known as PEG-DMG, obtainable from Avanti Polar Lipids, Alabaster, Ala.), and a phytosterol (optionally including a structural lipid such as cholesterol) at concentrations of about, e.g., 50 mM in a solvent, e.g., ethanol. Solutions should be refrigerated for storage at, for example, −20° C. Lipids are combined to yield desired molar ratios (see, for example, Table 6 below) and diluted with water and ethanol to a final lipid concentration of e.g., between about 5.5 mM and about 25 mM. Phytosterol* in Table 6 refers to phytosterol or optionally a combination of phytosterol and structural lipid such as beta-phytosterol and cholesterol.

TABLE 6
Exemplary formulations of LNP compositions

| Composition (mol %) | Components |
|---|---|
| 40:20:38.5:1.5 | Ionizable |
| | lipid:Phospholipid:Phytosterol*:PEG-DMG |
| 45:15:38.5:1.5 | Ionizable |
| | lipid:Phospholipid:Phytosterol*:PEG-DMG |
| 50:10:38.5:1.5 | Ionizable |
| | lipid:Phospholipid:Phytosterol*:PEG-DMG |
| 55:5:38.5:1.5 | Ionizable |
| | lipid:Phospholipid:Phytosterol*:PEG-DMG |
| 60:5:33.5:1.5 | Ionizable |
| | lipid:Phospholipid:Phytosterol*:PEG-DMG |
| 45:20:33.5:1.5 | Ionizable |
| | lipid:Phospholipid:Phytosterol*:PEG-DMG |
| 50:20:28.5:1.5 | Ionizable |
| | lipid:Phospholipid:Phytosterol*:PEG-DMG |
| 55:20:23.5:1.5 | Ionizable |
| | lipid:Phospholipid:Phytosterol*:PEG-DMG |
| 60:20:18.5:1.5 | Ionizable |
| | lipidd:Phospholipid:Phytosterol*:PEG-DMG |
| 40:15:43.5:1.5 | Ionizable |
| | lipid:Phospholipid:Phytosterol*:PEG-DMG |
| 50:15:33.5:1.5 | Ionizable |
| | lipid:Phospholipid:Phytosterol*:PEG-DMG |
| 55:15:28.5:1.5 | Ionizable |
| | lipid:Phospholipid:Phytosterol*:PEG-DMG |
| 60:15:23.5:1.5 | Ionizable |
| | lipid:Phospholipid:Phytosterol*:PEG-DMG |
| 40:10:48.5:1.5 | Ionizable |
| | lipid:Phospholipid:Phytosterol*:PEG-DMG |
| 45:10:43.5:1.5 | Ionizable |
| | lipid:Phospholipid:Phytosterol*:PEG-DMG |
| 55:10:33.5:1.5 | Ionizable |
| | lipid:Phospholipid:Phytosterol*:PEG-DMG |
| 60:10:28.5:1.5 | Ionizable |
| | lipidd:Phospholipid:Phytosterol*:PEG-DMG |
| 40:5:53.5:1.5 | Ionizable |
| | lipid:Phospholipid:Phytosterol*:PEG-DMG |
| 45:5:48.5:1.5 | Ionizable |
| | lipid:Phospholipid:Phytosterol*:PEG-DMG |
| 50:5:43.5:1.5 | Ionizable |
| | lipid:Phospholipid:Phytosterol*:PEG-DMG |
| 40:20:40:0 | Ionizable |
| | lipid:Phospholipid:Phytosterol*:PEG-DMG |
| 45:20:35:0 | Ionizable |
| | lipid:Phospholipid:Phytosterol*:PEG-DMG |
| 50:20:30:0 | Ionizable |
| | lipidd:Phospholipid:Phytosterol*:PEG-DMG |
| 55:20:25:0 | Ionizable |
| | lipid:Phospholipid:Phytosterol*:PEG-DMG |
| 60:20:20:0 | Ionizable |
| | lipid:Phospholipid:Phytosterol*:PEG-DMG |
| 40:15:45:0 | Ionizable |
| | lipid:Phospholipid:Phytosterol*:PEG-DMG |
| 45:15:40:0 | Ionizable |
| | lipid:Phospholipid:Phytosterol*:PEG-DMG |
| 50:15:35:0 | Ionizable |
| | lipid:Phospholipid:Phytosterol*:PEG-DMG |
| 55:15:30:0 | Ionizable lipidPhospholipid:Phytosterol*:PEG- |
| | DMG |
| 60:15:25:0 | Ionizable |
| | lipid:Phospholipid:Phytosterol*:PEG-DMG |
| 40:10:50:0 | Ionizable |
| | lipid:Phospholipid:Phytosterol*:PEG-DMG |
| 45:10:45:0 | Ionizable lipidPhospholipid:Phytosterol*:PEG- |
| | DMG |
| 50:0:48.5:1.5 | Ionizable |
| | lipid:Phospholipid:Phytosterol*:PEG-DMG |
| 50:10:40:0 | Ionizable |
| | lipid:Phospholipid:Phytosterol*:PEG-DMG |
| 55:10:35:0 | Ionizable |
| | lipid:Phospholipid:Phytosterol*:PEG-DMG |
| 60:10:30:0 | Ionizable lipidPhospholipid:Phytosterol*:PEG- |
| | DMG |

Nanoparticle compositions including a therapeutic and/or prophylactic and a lipid component are prepared by combining the lipid solution with a solution including the therapeutic and/or prophylactic at lipid component to therapeutic and/or prophylactic wt:wt ratios between about 5:1 and about 50:1. The lipid solution is rapidly injected using a NanoAssemblr microfluidic based system at flow rates between about 10 ml/min and about 18 ml/min into the therapeutic and/or prophylactic solution to produce a suspension with a water to ethanol ratio between about 1:1 and about 4:1.

For nanoparticle compositions including an RNA, solutions of the RNA at concentrations of 0.1 mg/ml in deionized water are diluted in a buffer, e.g., 50 mM sodium citrate buffer at a pH between 3 and 4 to form a stock solution.

Nanoparticle compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, IL) with a molecular weight cutoff of 10 kDa. The first dialysis is carried out at room temperature for 3 hours. The formulations are then dialyzed overnight at 4° C. The resulting nanoparticle suspension is filtered through 0.2 ₁.tm sterile filters (Sarstedt, Numbrecht, Germany) into glass vials and sealed with crimp closures. Nanoparticle composition solutions of 0.01 mg/ml to 0.10 mg/ml are generally obtained.

The method described above induces nano-precipitation and particle formation. Alternative processes including, but not limited to, T-junction and direct injection, may be used to achieve the same nano-precipitation.

### B. Characterization of Nanoparticle Compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1 xPBS in determining particle size and 15 mM PBS in determining zeta potential.

Ultraviolet-visible spectroscopy can be used to determine the concentration of a therapeutic and/or prophylactic (e.g., RNA) in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, Calif.). The concentration of therapeutic and/or prophylactic in the nanoparticle composition can be calculated based on the extinction coefficient of the therapeutic and/or prophylactic used in the composition and on the difference between the absorbance at a wavelength of, for example, 260 nm and the baseline value at a wavelength of, for example, 330 nm.

For nanoparticle compositions including an RNA, a QUANT-IT™ RIBOGREEN® RNA assay (Invitrogen Corporation Carlsbad, Calif.) can be used to evaluate the encapsulation of an RNA by the nanoparticle composition. The samples are diluted to a concentration of approximately 5 μg/mL in a TE buffer solution (10 mM Tris-HCl, mM EDTA, pH 7.5). 50 μL of the diluted samples are transferred to a polystyrene 96 well plate and either 50 μL of TE buffer or 50 μL of a 2% Triton X-100 solution is added to the wells. The plate is incubated at a temperature of 37° C. for 15 minutes. The RIBOGREEN® reagent is diluted 1:100 in TE buffer, and 100 μL of this solution is added to each well. The fluorescence intensity can be measured using a fluorescence plate reader (Wallac Victor 1420 Multilablel Counter; Perkin Elmer, Waltham, Mass.) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### C. In Vivo Formulation Studies

In order to monitor how effectively various nanoparticle compositions deliver therapeutic and/or prophylactics to targeted cells, different nanoparticle compositions including a particular therapeutic and/or prophylactic (for example, a modified or naturally occurring RNA such as an mRNA) are prepared and administered to rodent populations. Mice are intravenously, intramuscularly, subcutaneously, intraarterially, or intratumorally administered a single dose including a nanoparticle composition with a lipid nanoparticle formulation. In some instances, mice may be made to inhale doses. Dose sizes may range from 0.001 mg/kg to 10 mg/kg, where 10 mg/kg describes a dose including 10 mg of a therapeutic and/or prophylactic in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. For nanoparticle compositions including mRNA, time courses of protein expression can also be evaluated. Samples collected from the rodents for evaluation may include blood, sera, and tissue (for example, muscle tissue from the site of an intramuscular injection and internal tissue); sample collection may involve sacrifice of the animals.

Nanoparticle compositions including mRNA are useful in the evaluation of the efficacy and usefulness of various formulations for the delivery of therapeutic and/or prophylactics. Higher levels of protein expression induced by administration of a composition including an mRNA will be indicative of higher mRNA translation and/or nanoparticle composition mRNA delivery efficiencies. As the non-RNA components are not thought to affect translational machineries themselves, a higher level of protein expression is likely indicative of a higher efficiency of delivery of the therapeutic and/or prophylactic by a given nanoparticle composition relative to other nanoparticle compositions or the absence thereof.

### Example 2

### Tethered eIF4G Increases Potency of Target mRNA

This Example describes increased potency of a target mRNA (e.g., increased protein expression and/or duration of protein expression) when co-delivered with an RNA encoding a tethered effector protein, e.g., tethered eIF4G.

The system used in this Example is depicted in FIG. 1. The eIF4G mid-to-C-terminal domain can support ribosome complex formation, support or enhance cap-dependent translation when recruited to mRNA 3′ UTR via cap-independent translation enhancers (CITEs), and stimulate translation of a capless mRNA when recruited via IRES elements (Kraft J J, et al. (2013) NAR. 41(5):3398-413; Paek K Y, et al. (2015) PNAS 112(4):1041-6; Pestova T V, et al. (1996) Mol Cell Bio1.16(12):6870-8). Thus, this domain (herein named eIF4GΔN) was used as the effector. With no eIF4E and PABP binding abilities, this domain may not bind other mRNAs in the cell, thereby reducing probable off-target effects. As a tether the MS2-MBP system was used since it has been extensively validated over the last few decades. Notably the MS2-MBP interaction is expected to be very strong (K_{d}˜1-10 nM) and stable in a cell (Tutucci E, et al. (2018) Nat Methods. 15(1):81-89). Six MS2 loops were inserted in the 3′ UTR of the target mRNA. The MS2 loops either replace the 3′ UTR completely or are added before or after v1.1_UTR sequence (FIG. 1).

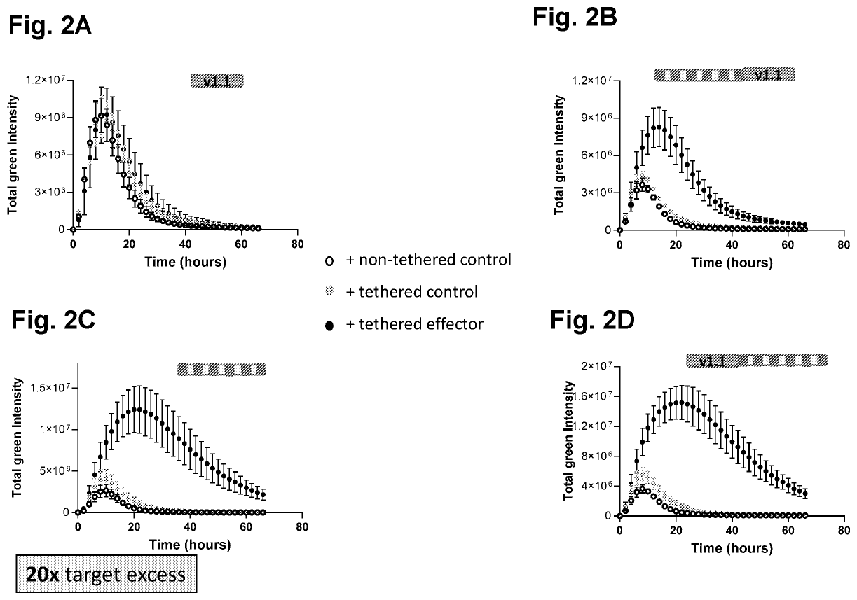

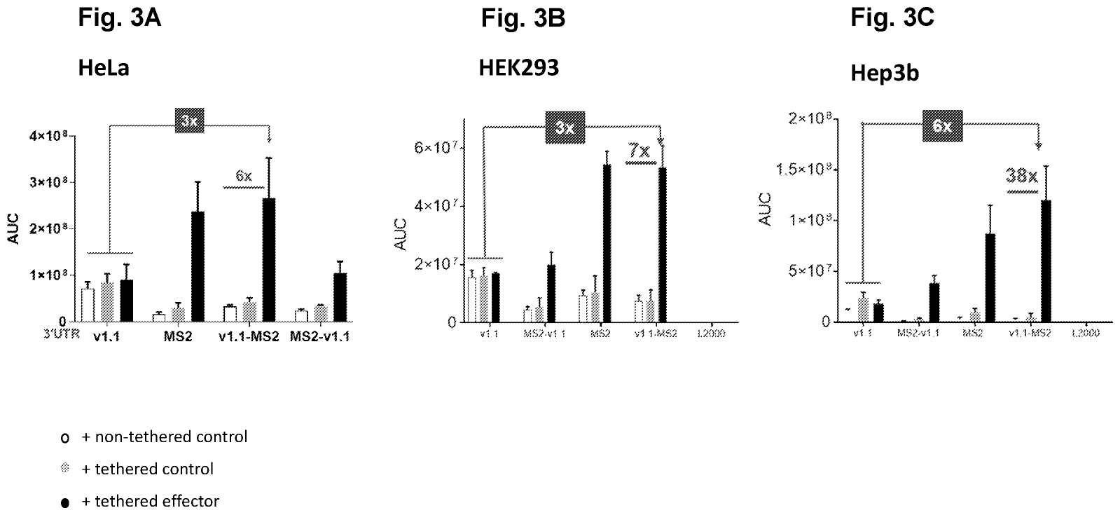

Initial cell-based experiments relied on using deg-GFP as the reporter in HeLa cells. Target reporter RNA was co-delivered with an mRNA encoding control protein, EPO; or an mRNA encoding tethered control protein, MBP-LacZ; or an mRNA encoding tethered effector protein, MBP-eIF4GΔN. The experiments were done in 10× molar excess of target RNA. As expected, minimal differences were observed in target mRNA expression with 3′v1.1_UTR under different conditions (this RNA has no binding site for any of the encoded proteins). In all 3 instances of target mRNAs that have binding sites for the tethered protein, tethered effector significantly increased protein output from the mRNA (about 3-6x increase in protein output). Maximum benefits were observed when the tether was immediately adjacent to the polyA sequence (FIGS. 2A-2D). This boost in expression was reproduced across multiple cell-types (FIGS. 3A-3C) ranging from 3-fold (modest) to 80-fold (high).

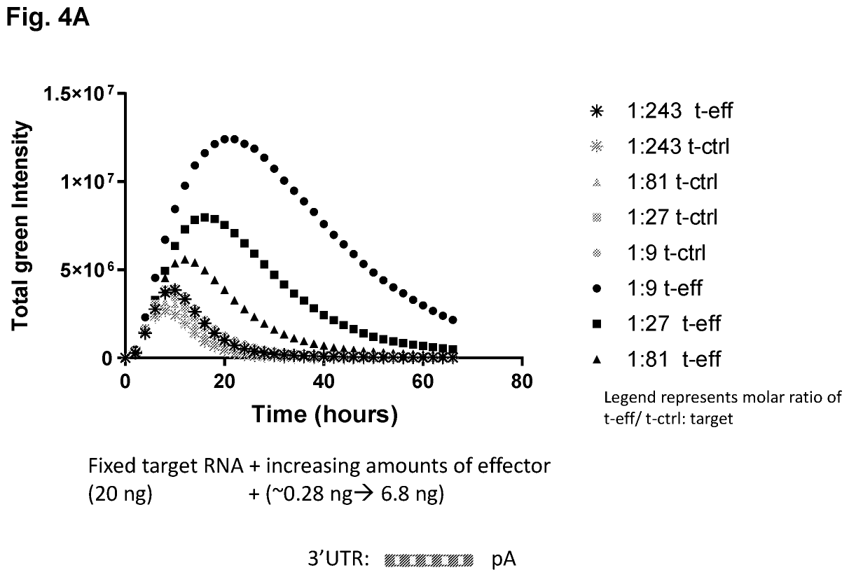

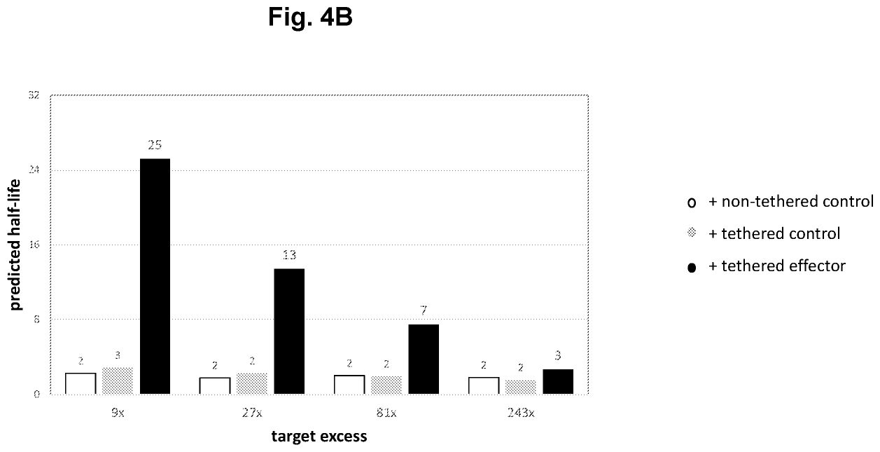

Finally, at fixed target mRNA amount, the potency boost with tethered effector increased with increasing amounts of effector. This was also evident when the data was fitted to a half-life model that predicted half-life of target mRNA increased with increasing effector concentrations (FIGS. 4A-4B). At a 9× molar excess of target, ˜8-10 fold increase in half-life of the target mRNA was observed in cells delivered the target mRNA with the tethered effector (green bars), as compared to cells delivered the target m RNA with a tethered control (orange bars) (see FIG. 4B).

The boost in protein expression with tethered effector was observed for two more reporters, Luc and NPI-Luc. The shapes of the total-intensity curves and model fits both suggest an increased half-life for the target mRNA in the presence of tethered effector protein.

In summary, the data provided in this experiment demonstrates that the expression of a target mRNA and its encoded protein can be significantly increased when said target mRNA is co-delivered with a tethered effector protein.

### Example 3

### Tethered eIF4G Increases mRNA Half-Life

This Example describes increased half-life of a target mRNA when co-delivered with a tethered effector, e.g., tethered eIF4G.

A similar system as used in Example 2 was used in this Example. Briefly, HEK293 cells were electroporated with target reporter RNA and an mRNA encoding control protein (EPO); or an mRNA encoding tethered control protein (MBP-LacZ); or an mRNA encoding tethered effector protein (MBP-eIF4GΔN). The experiments were done in 3× molar excess of target RNA.

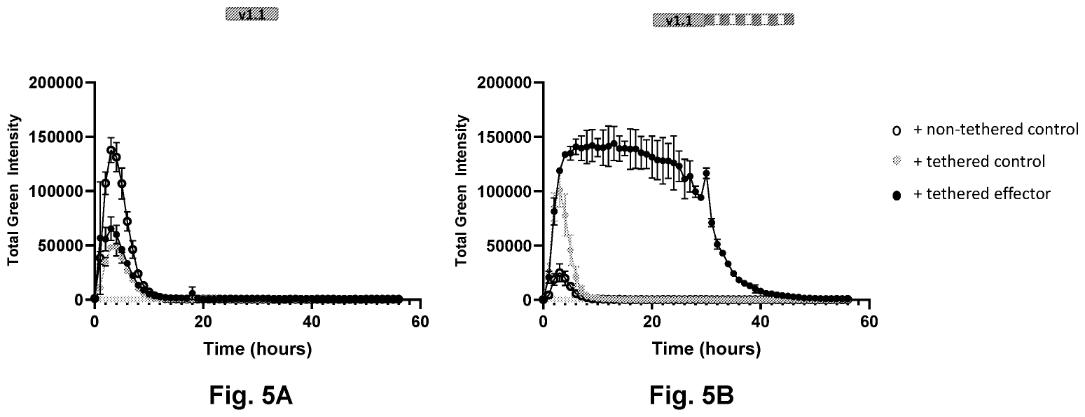

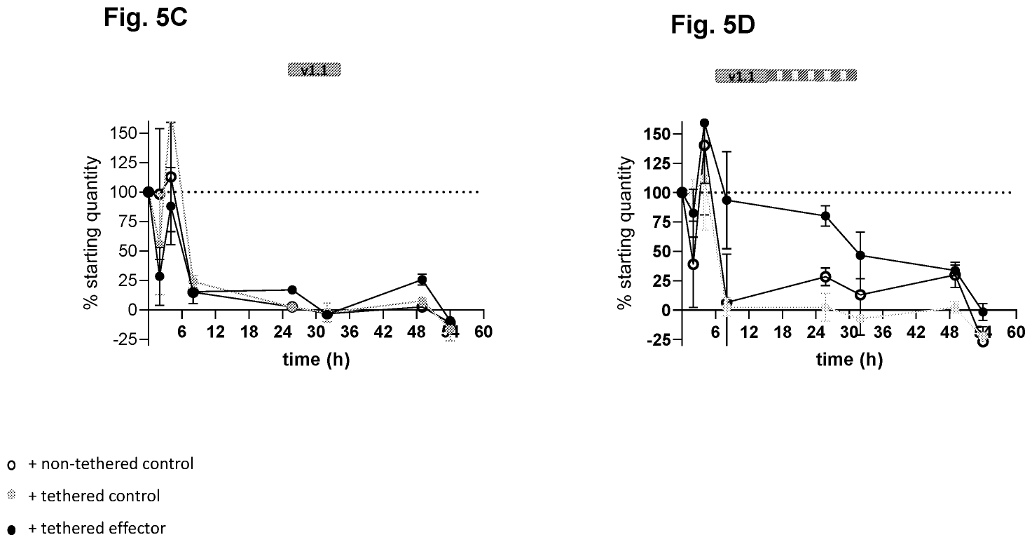

As shown in FIGS. 5C-5D, half-life of the target mRNA was increased in the presence of the tethered effector as compared to the tethered control or non-tethered control. Maximum benefits were observed when the tether was immediately adjacent to the polyA sequence. FIG. 5D shows that about 80% of the target mRNA (co-delivered with the tethered effector) is present/can be detected at 24 hours post electroporation compared to control conditions (target m RNA with tethered control or with non-tethered control).

This data shows that the half-life of a target mRNA can be increased when said target mRNA is co-delivered with a tethered effector, e.g., tethered eIF4G.

### Example 4

### Evaluation of Target mRNA Translation with Tethered Effector

This Example describes the effects of a tethered effector on the translation of target mRNA.

For this Example, the nascent peptide imaging (NPI)-Luc reporter system (used to image mRNA, translating mRNA and protein) was utilized to evaluate target mRNA translation with a tethered effector. The NPI-Luc reporter encodes for a Luc ORF tagged with V5 peptide on the N-terminal end, and a nuclear localization signal. In using this reporter, by assessing spots/signal positive for both V5 (newly forming protein) and smFISH (that detects LUC RNA), one is able to measure translating mRNAs. This is done by assessing the spots that are positive for both NPI+(or V5 positive) smFISH+where NPI reflects the nascent peptide, and the FISH probes are specific for Luc.

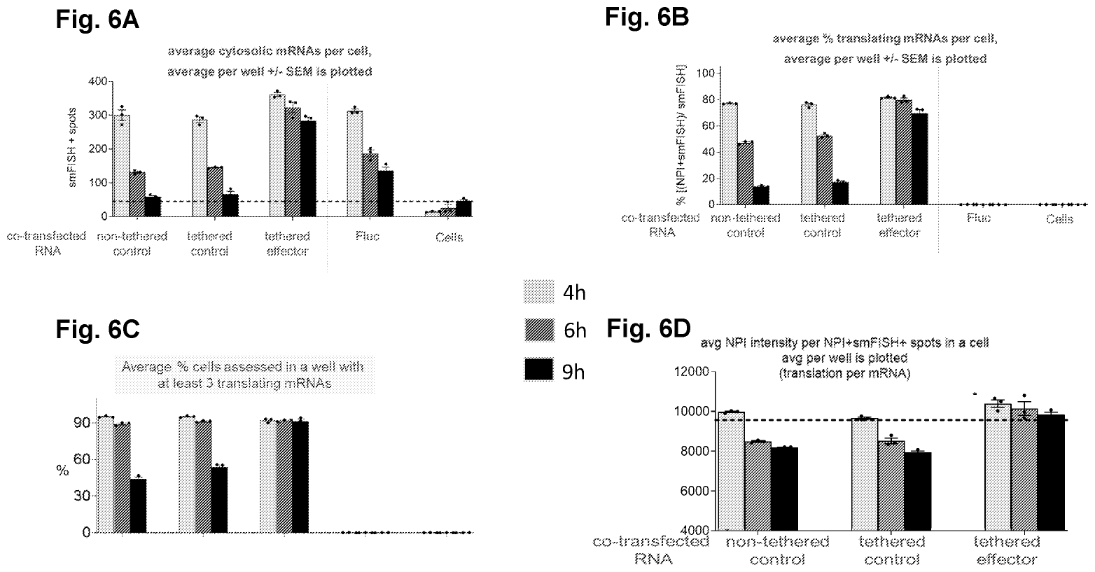

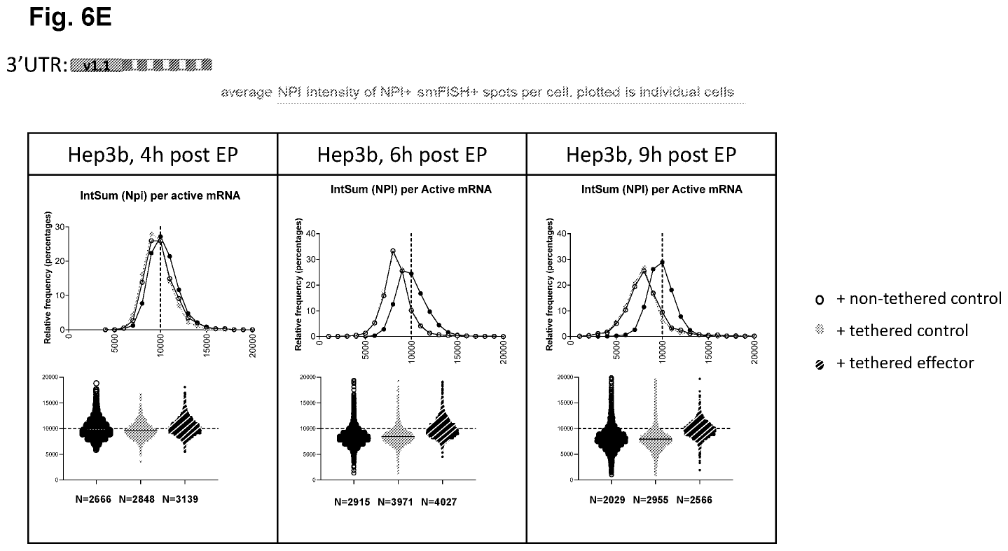

All experiments were done with target or control RNA electroporation under different conditions. In HeLa, imaging results were obtained for target or control mRNA with non-tethered, tethered control, and tethered effector RNAs (only images for tethered control and effector RNA groups are shown). Tethered effector exhibited higher translation (colocalized NPI+smFISH+spots) for MS2 containing target RNAs, especially at later time points in HeLa. This experiment was repeated in HeLa and Hep3b with NPI-Luc mRNA with MS2 binding sites in the UTR. The quantified data is shown in FIGS. 6A-6E, 7A-7D, 16 and 17A-17C.

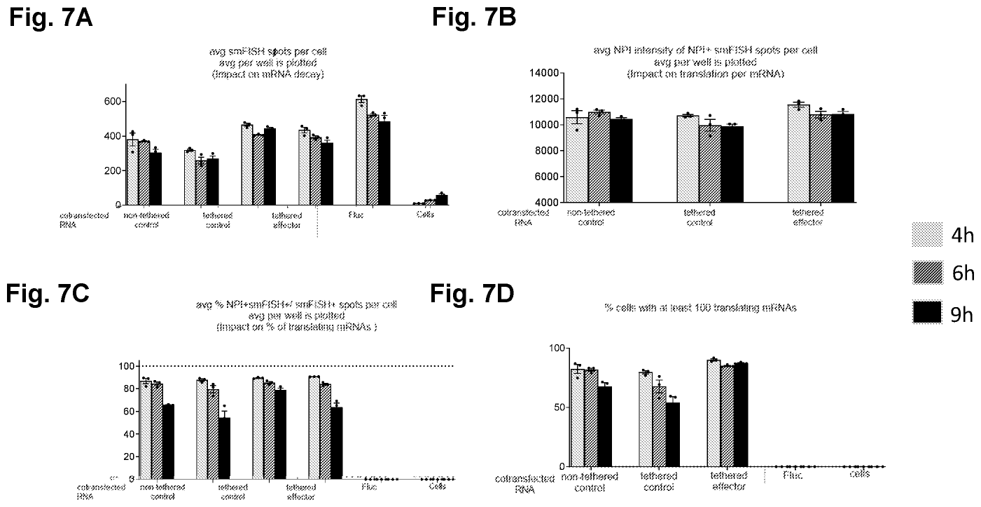

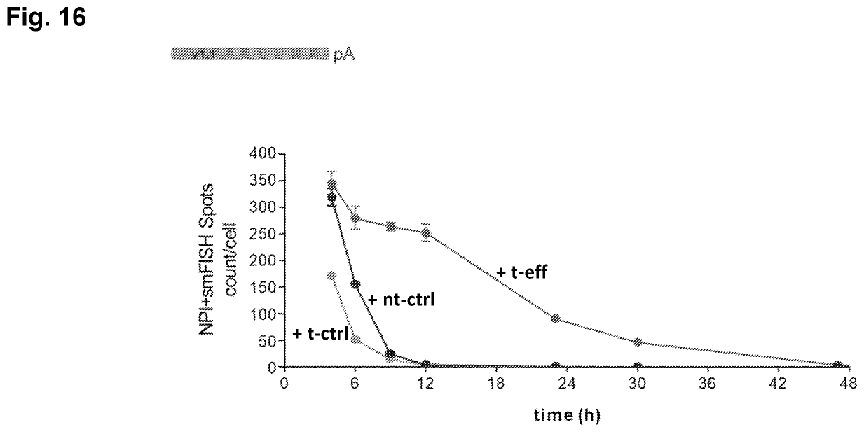

In Hep3b cells, co-transfection with effector RNA led to i) decreased mRNA loss with time; ii) decreased translating mRNA loss with time; iii) robust translation maintenance in cells over time and iv) translation maintenance per mRNA over time(FIGS. 6A-6E). FIG. 16 shows that the tethered effector decreases loss of translating mRNAs over a time period of 48 hours post transfection. In HeLa cells, translation overall appeared more permissive. A minimal impact was observed on cytosolic mRNAs with time, and translation per mRNA with time at any of the time points under any condition. However, tethered effector reduced the loss of translating mRNAs with time and showed more robust translation in a higher fraction of cells with time (FIGS. 7A-7D). The difference in magnitude of impact observed could be cell specific, or could depend on the dynamic range of the assay in the two cell types.

In summary, the data provided in this Example shows that a tethered effector can prevent loss of mRNA with time; prevent a reduction in, or maintain translation on mRNAs with time; and promote translation in cells over time. Therefore, in some embodiments, co-delivery of a tethered effector and target mRNA can be used to increase the translation output of a target mRNA or increase the duration of translation output from an mRNA, thereby increasing the amount of target protein.

### Example 5

### Identifying the Domain of eIF4G Required for Effector Function

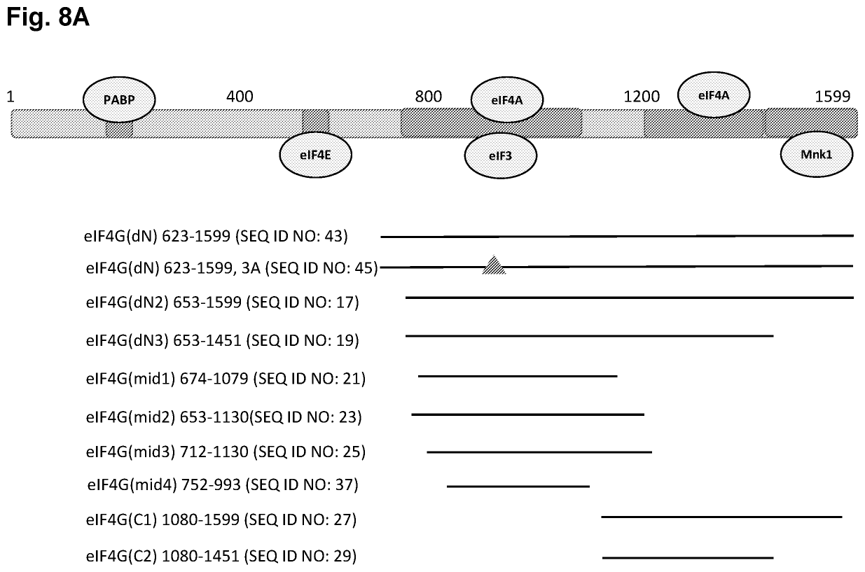

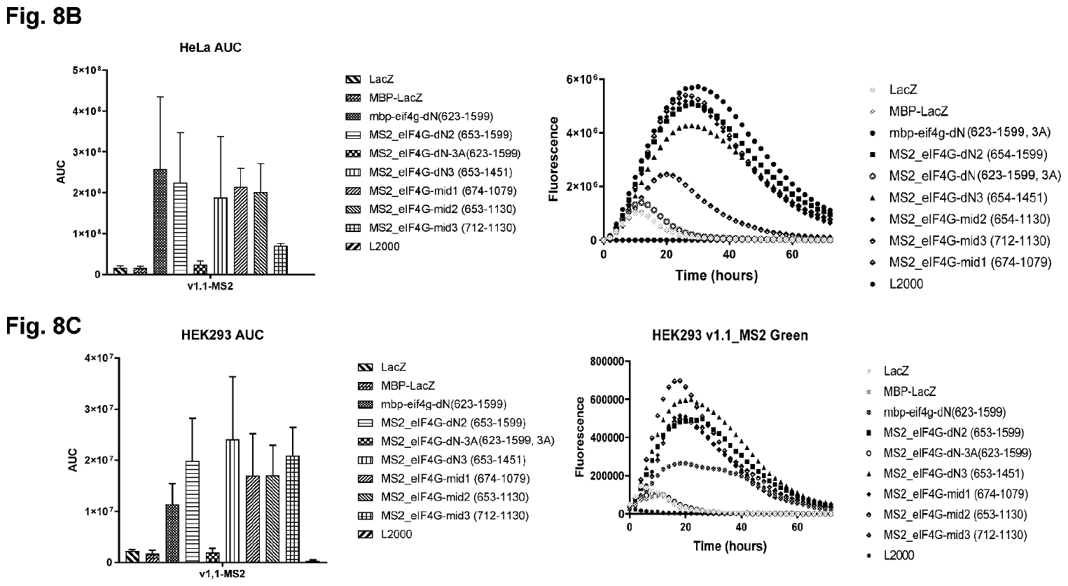

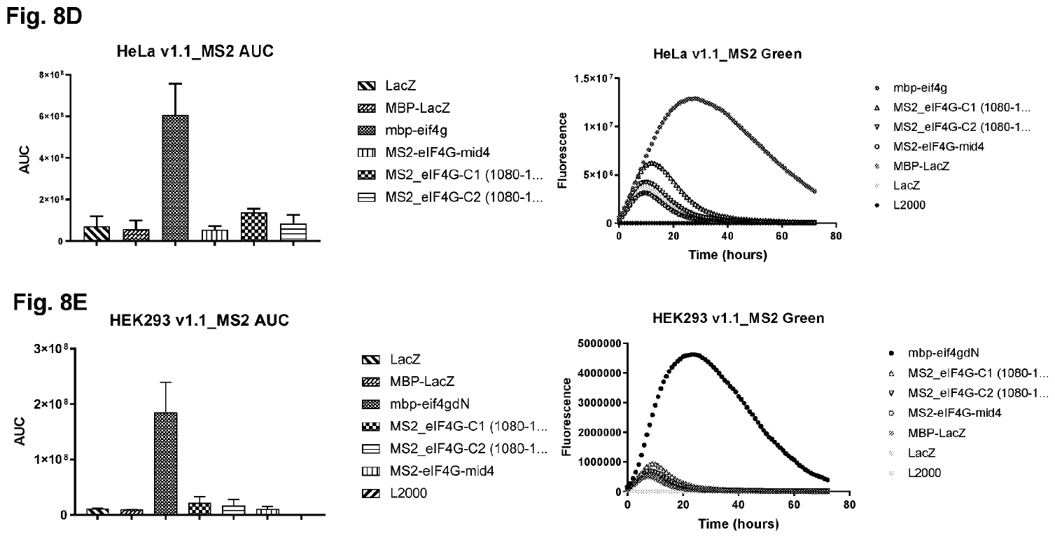

This Example describes an analysis of the domains of eIF4G which are required for effector function. The schematic of the different constructs used in this Example is provided in FIG. 8A.

Target deg-GFP RNA was co-delivered with an mRNA encoding control protein, EPO; or an mRNA encoding tethered control protein, MBP-LacZ; or an mRNA encoding tethered effector protein, MBP-eIF4GΔN, or mRNAs encoding MBP-fused to eIF4G truncations and mutations as illustrated in FIG. 8A. The experiment was done in 10× molar excess of target RNA. As expected, minimal differences were observed in target mRNA expression with 3′v1.1_UTR under different conditions.

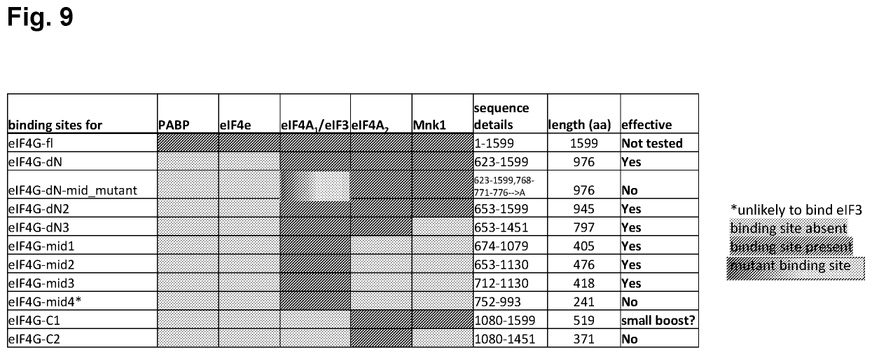

As shown in FIGS. 8B-8E, the eIF4G dN, eIF4G dN2, eIF4G dN3, eIF4G mid1, eIF4G mid2, eIF4G mid3, eIF4G mid4, eIF4G C1 and eIF4G C2 constructs resulted in increased protein output from the target mRNA with MS2-binding sites in the UTR. In contrast FIGS. 8B and 8D show that the eIF4G dN 3A construct (which is unable to bind eIF3 and eIF4A through the mid-domain (Imataka H, Sonenberg N. (1997) Mol Cell Bio1.17(12):6940-7)) showed no appreciable benefit over tethered control. FIG. 9 provides a summary of expected binding activity for the various constructs that were tested.

Taken together, this data shows that ribosome binding of eIF4G via EIF3/EIF4A is important for effector function.

### Example 6

### Tethered eIF4G Rescues Protein Expression and Increases mRNA Stability

This Example describes increasing the protein expression (or stability) of an unstable/translationally inactive target mRNA when co-delivered with an RNA encoding a tethered effector.

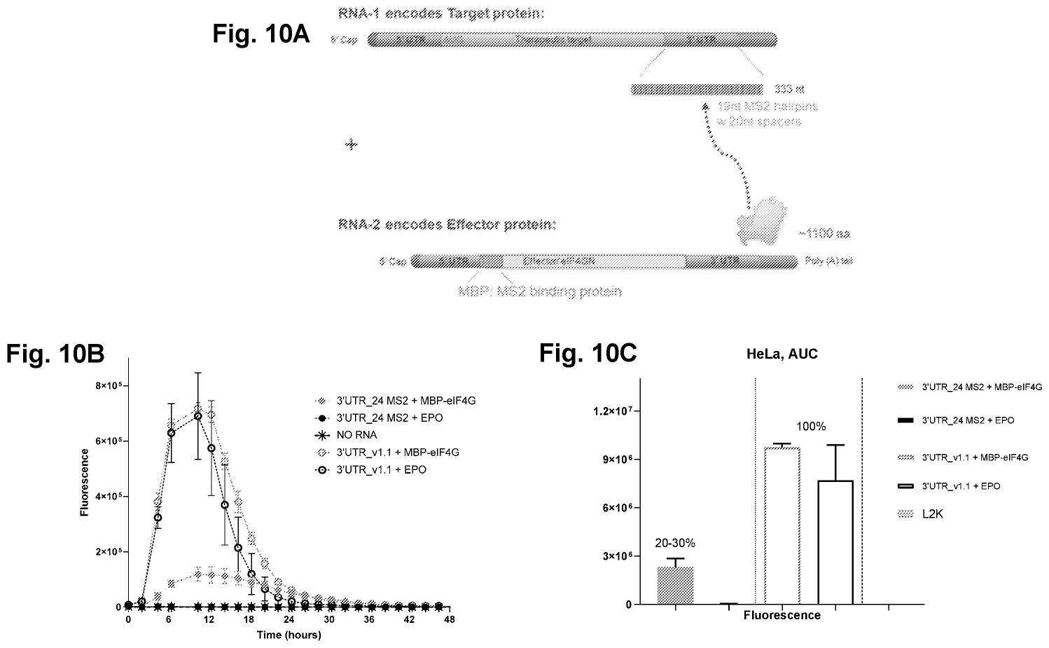

A similar system as used in Example 2 was used in this Example. HeLa cells were electroporated with 2 RNAs. Each sample has target degGFP encoding RNA co-transfected with a control (EPO) (i.e., 3′UTR +EPO) or effector protein encoding RNA (i.e., 3′UTR +MBP-eIF4G). The experiments were done in 3× molar excess of target RNA. The 3′ UTR used is either 3′UTR with 24 MS2 hairpins (3′UTR 24 MS2) or 3′ UTR v1.1. As shown in FIG. 10B, an increased number of MS2 hairpin loops (24 MS2) in the 3′ UTR of the target RNA reduced protein expression (and possibly the mRNA half life) compared to the 3′ UTR v1.1. However, mRNA expression was restored even for the mRNA with 24 MS2 loop, when it was tethered to an activator protein (eIF4G1(623-1599)) using the MS2-binding protein. In addition, there was a notable extension of the functional half-life of the target RNA in the presence of the activator protein. FIG. 10C shows the area under the curve (AUC) for the same data in FIG. 10B.

This data shows that the protein expression (and possibly half-life of a target mRNA) can be increased when the target mRNA is co-delivered with an RNA encoding a tethered effector.

### Example 7

### Tailless Target RNAs Can be Rescued by Effectors Recruited Using RBP-RNA (MBP-MS2) Tethers

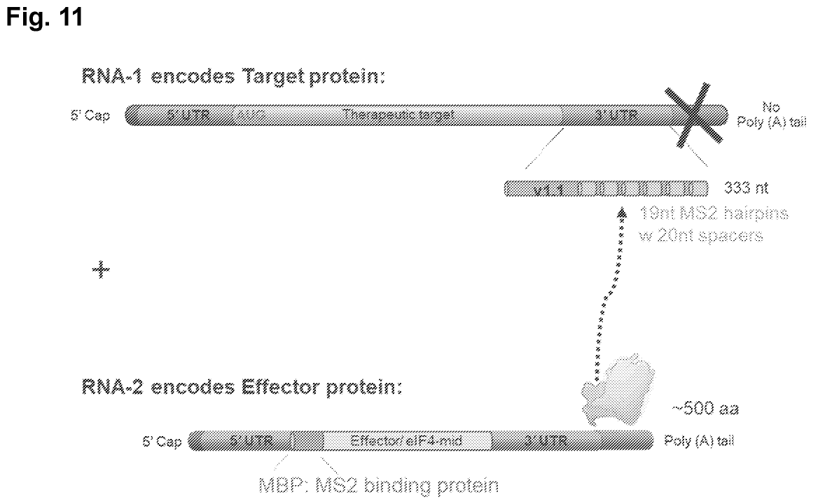

FIG. 11 provides a schematic of a system to recruit potential effectors to target RNA with an MS2 binding protein (MBP)-MS2 tether to rescue tailless target RNA. The target mRNA has MS2 loops in the 3′UTR and lacks a polyA tail (A0). The second mRNA encodes MS2-binding protein (MBP) fused to an effector/eIF4G-mid. The MBP-MS2 binding interaction recruits the effector protein to the target RNA. This system can be coupled to a miRNA-dependent switch gate to permit tethering in specific cells, thereby turning ON expression of the target protein. This system can also be coupled to an effector encoding DNA molecule under the control of a tissue-specific promoter to turn on expression in specific cells. Finally this system can be encoded such that the effector protein expression and/or recruitment is under the control of a trigger (receptor-mediated activation, a change in pH, hypoxia, etc) in specific microenvironments and/or specific cell types (the presence of specific microRNA in that cell-type). Thus, in some embodiments, even if the effector is made, it can not be recruited to the target RNA without a specific trigger.

The eIF4G mid-to-C-terminal domain can support ribosome complex formation, support or enhance cap-dependent translation when recruited to mRNA 3′ UTR via cap-independent translation enhancers (CITEs), and stimulate translation of a capless mRNA when recruited via IRES elements (Kraft J J, et al. (2013) NAR. 41(5):3398-413; Paek K Y, et al. (2015) PNAS 112(4):1041-6; Pestova T V, et al. (1996) Mol Cell Biol.16(12):6870-8). Thus, this domain (herein named eIF4GΔN) can be used as the effector. The domain eIF4-mid can also be used as the effector. With no eIF4E and PABP binding abilities, this domain may not bind other mRNAs in the cell, thereby reducing probable off-target effects. As a tether, the MS2-MBP system can be used since it has been extensively validated over the last few decades. Notably the MS2-MBP interaction is expected to be very strong (K_{d}˜1-10 nM) and stable in a cell (Tutucci E, et al. (2018) Nat Methods. 15(1):81-89). Six MS2 loops were inserted in the 3′UTR of the target mRNA.

### Example 8

### Tethered eIF4G Increases mRNA Stability of Tailless mRNA

This Example describes increasing translation and stability of a tailless mRNA when co-delivered with a tethered effector.

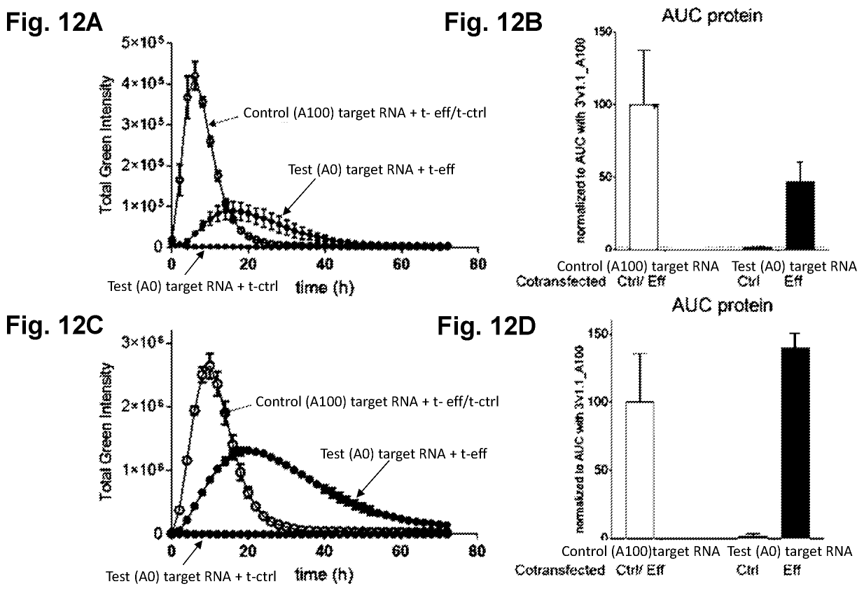

Hep3b cells or HeLa cells were transfected with 2 RNAs. Each sample has a target degGFP co-transfected with an mRNA encoding a tethered protein (Effector, eIF4G-mid2) or a tethered control (MBP-LacZ). The target mRNA was tailless (A0) (i.e., 3′v1.1_MS2 AO) or has an A100 tail (v1.1_A100). The experiments were done in 10× molar excess of target RNA and protein expression was measured by Incucyte. As shown in FIGS. 12A and 12C, while standard mRNAs with A100 tails show robust expression, no detectable expression is seen for tailless mRNAs with MS2 UTRs co-delivered with tethered control (Test (A0) target RNA+t-ctrl). The translation of tailless targets is restored in the presence of the eIF4G mid domain (Test (A0) target RNA+t-eff). The shape of the curve suggests an increase in RNA half-life. FIGS. 12B and 12D show the area under the curve (AUC) for the same data in FIGS. 12A and 12B.

### Example 9

### Tailless Target RNAs with idT Tails Can be Rescued by Effectors Recruited Using RBP-RNA (MBP-MS2) Tethers

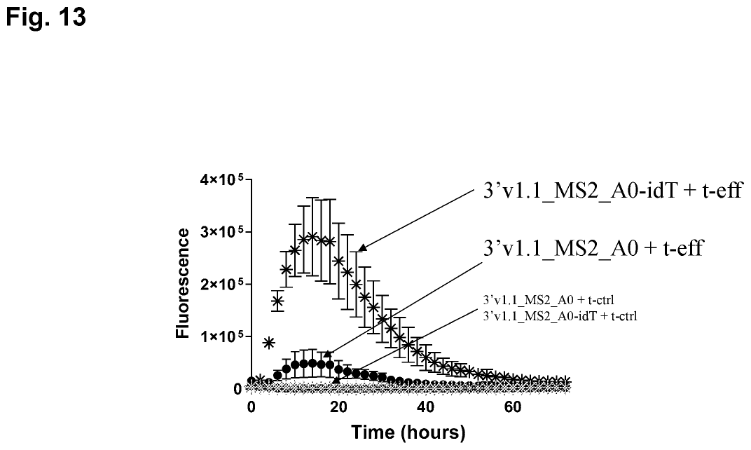

To illustrate that the system of FIG. 11 can be used to rescue modified tailless target RNA expression, HEP3B cells were transfected with 3′v1.1_MS2_A0 or 3′v1.1_MS2 A0-idT +Effector/Control deg-GFP constructs. In this experiment, tailless target RNA was modified with an idT tail, i.e., A20 oligo followed by an inverted idT. Control RNA encodes for MBP-LacZ and effector RNA encodes for MBP-eIF4G-mid. Target RNA was in 10× molar excess of effector RNA. As seen in FIG. 13, A0 or A0-idT constructs showed no detectable expression when co-transfected with control RNA. Tethering effector increased expression from A0 RNA. Tethering in the presence of idT rescued expression to a much higher level. These results demonstrate that translation from an AO RNA can improve in the presence of idT, along with a tether.

### Example 10

### Capless Target RNAs Can be Rescued by Effectors Recruited Using RBP-RNA (MBP-MS2) Tethers

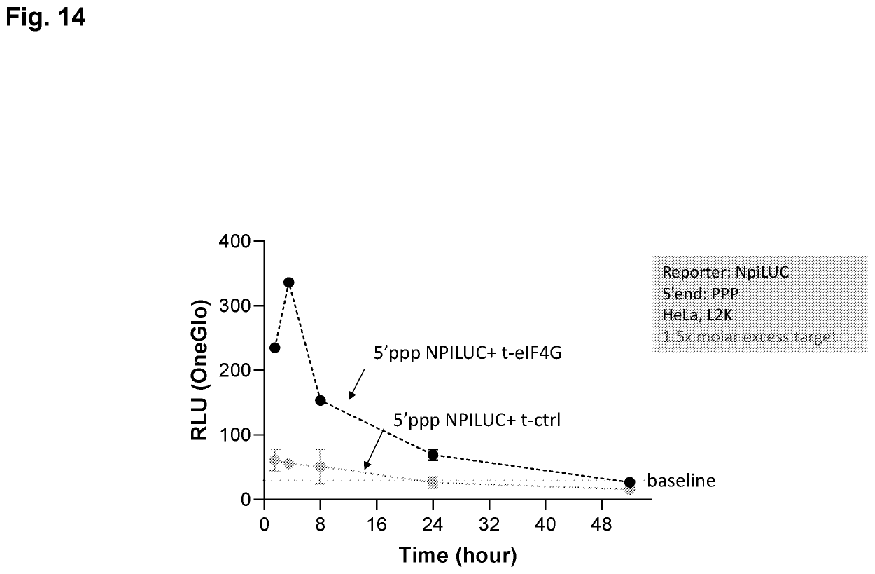

To illustrate that the system of FIG. 11 can be used to rescue capless target RNA, HeLa cells were transfected with 5′ triphosphate (5′ppp) ended NpiLuc-_3′v1.1_MS2 A100 target RNAs +Effector/Control NpiLuc constructs. Target RNA was in 1.5× molar excess of effector RNA. As seen in the FIG. 14, while the capless RNA by itself showed no appreciable signal above background with a control tether RNA (MBP-mid2-Mut, encodes for a mutant mid domain) , appreciable expression is restored on this RNA when tethered with effector (MBP-mid2)+Effector/Control deg-GFP construct, and 3′v1.1_MS2 AO +Control or Effector.

### Example 11

### Capless-Tailless Target RNAs can be Rescued by Effectors Recruited Using RBP-RNA (MBP-MS2) Tethers

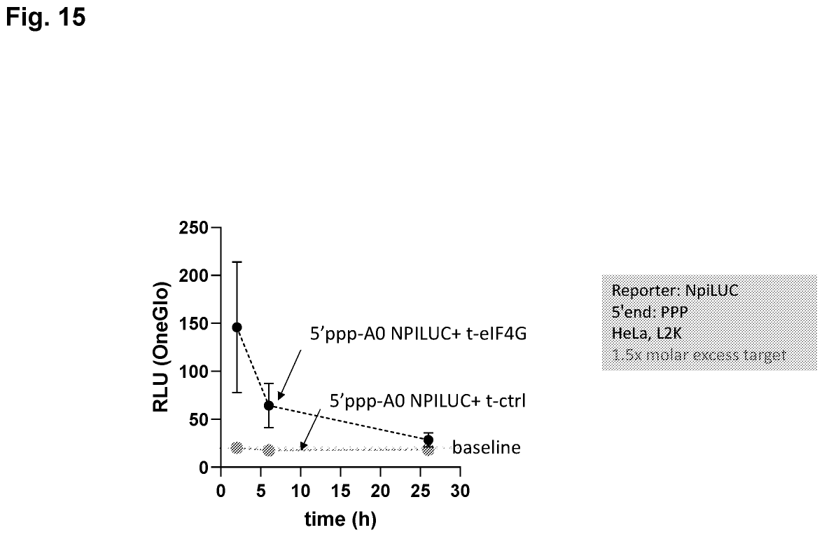

To illustrate that the system of FIG. 11 can be used to rescue capless-tailless target RNA, HeLa cells were transfected with 5′ppp ended NpiLuc 3′v1.1_MS2 A0 target RNAs+Effector/Control NpiLuc constructs. Target RNA was in 1.5× molar excess of effector RNA. As seen in the FIG. 15, while the capless-tailless RNA by itself showed no appreciable signal above background with a control tether RNA (MBP-mid2-Mut, encodes for a mutant mid domain), appreciable expression was restored on this RNA when delivered with the tethered-effector construct (MBP-mid2).

### Example 12

### Tethered Effector Maintains More Translating mRNAs in More Cells Over Time

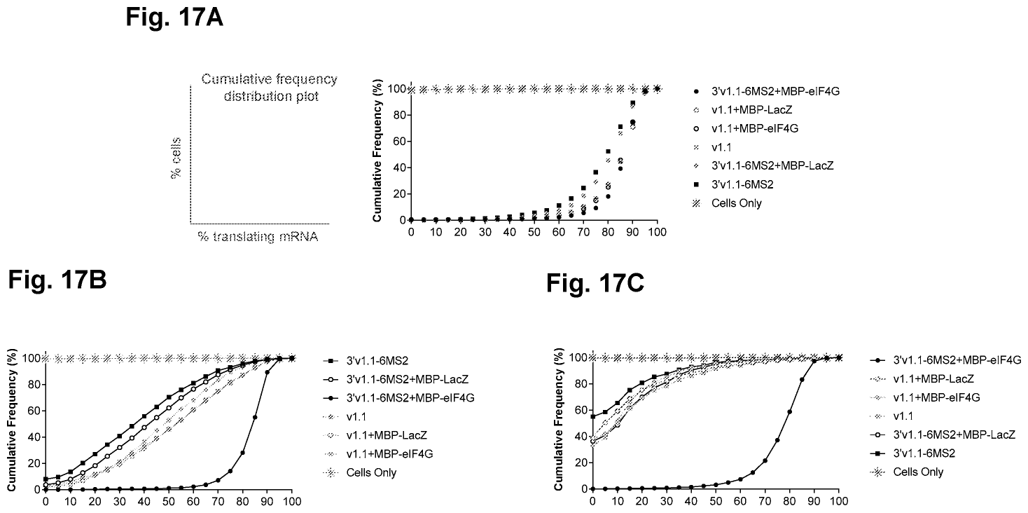

This Example describes maintenance of more translating mRNAs in more cells over time when the mRNA is co-delivered with a tethered effector, e.g., tethered eIF4GΔN. Hep3b cells were electroporated with the target RNA (3′v1.1MS2_A100 or 3′v1.1_A100) in combination with non-tethered control (nt-ctrl; LacZ), tethered control (t-ctrl; MBP-LacZ) or t-effector (eIF4GΔN). Tethered effector exhibited a higher percentage of translating mRNA (NPI+smFISH+spots) for a longer period of time, as seen in FIGS. 17A-17C.

### Example 13

### Tethering Increases Secreted Protein Expression and Translation

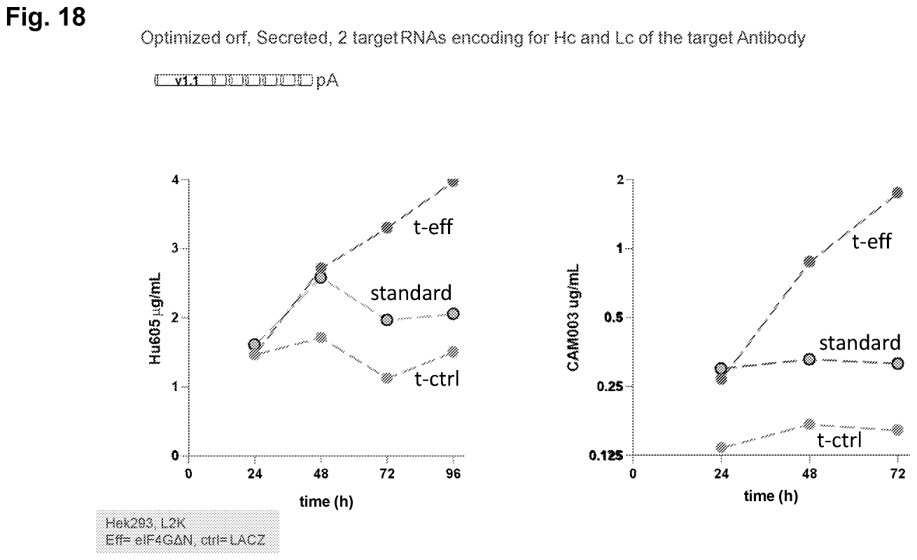

This Example demonstrates that secreted protein expression and translation is increased when the mRNA is co-delivered with a tethered effector, e.g., tethered eIF4GΔN. v1.1_target RNA constructs with optimized reading frames for the light chain and heavy chain pairs of two secreted antibodies, (Ab 1 and Ab2) were co-transfected into Hek293 cells with t-ctrl (MBP-LacZ) or t-eff (MBP-eIF4GΔN). Experiments were done with 5× molar target excess (Ab 1) or lx molar target excess (Ab2). In each of the antibody experiments, the tethered effector is tethered to two separate light and heavy chain encoding RNAs. FIG. 18 shows the concentration (μg/m1) of Ablor Ab2 over time and indicates that tethering increases secreted protein expression and translation.

### Example 14

### A Single RNA Tethering System Enhances Target Expression In Vivo

This Example demonstrates that a single RNA tethering system can increase the efficiency of target RNA translation in vivo. FIG. 19 provides a schematic of the single RNA tethering system. The mRNA molecule from 5′ to 3′ includes a CAP, a 5′UTR, target ORF, 3 protease cleavage sites in tandem: T2A-P2A-E2A/TPE (red), another ORF encoding for the RNA binding protein fused to an effector (orange-RBP, -green-Effector), and MS2 loops (orange stripes) in the 3′UTR. The MS2 loops are added after v1.1_UTR sequence. The TPE protease cleavage site leads to ribosome skipping during translation in a cell. The end product of this translation is that the target ORF encoded protein gets a C-terminal tag, and the RNA binding binding-effector fusion starts with a residual Proline (C-terminal amino acid) from TPE. For specifically TPE cleavage site, this arrangement is expected to lead to 1 Target: 0.05 effector protein molecules (Liu et al, 2017 Scientific Reports 7: 2193). In the 1 RNA system, the effector protein binding to the RNA 3′UTR via the RNA-binding-protein interaction confers the benefit of effector protein binding to this one RNA encoding for both target and effector ORFs.

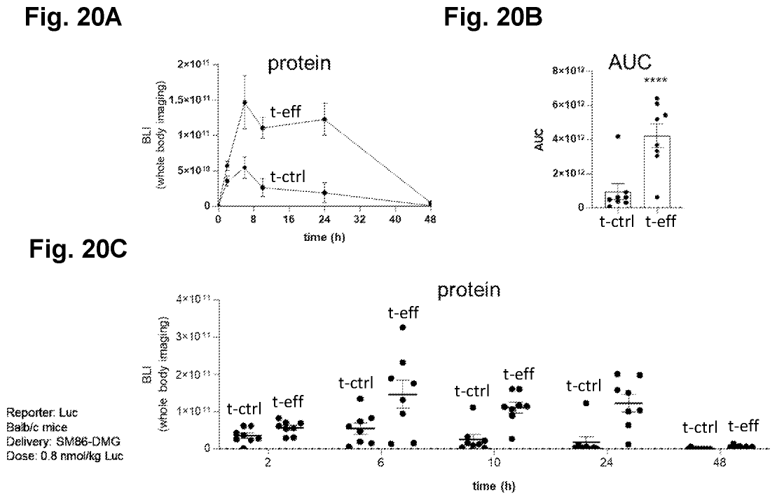

FIG. 20A shows the level of luminescence over the various timepoints indicated for mice that were injected with a t-eff construct (Effector) or a t-ctrl construct (Control). FIG. 20B shows the cumulative luminiscence plotted as total Area Under Curve; AUC) corresponding to FIG. 20B. FIG. 20C shows the luminescence over the time points indicated for mice that were injected with a t-eff construct (Effector) or a t-ctrl construct (Control). Data is shown as mean +/− SEM. The deliverey system used here is an LNP.

### Example 15

### Identifying Other Useful Effector Proteins or Domains Thereof

This Example describes an analysis of the homologues or domains of eIF4G which are required for effector function.

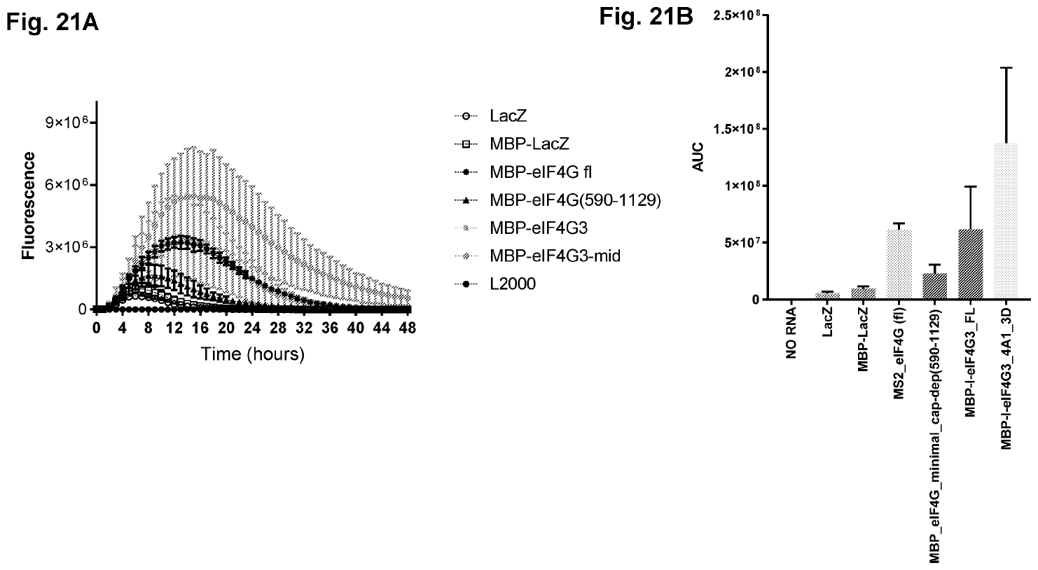

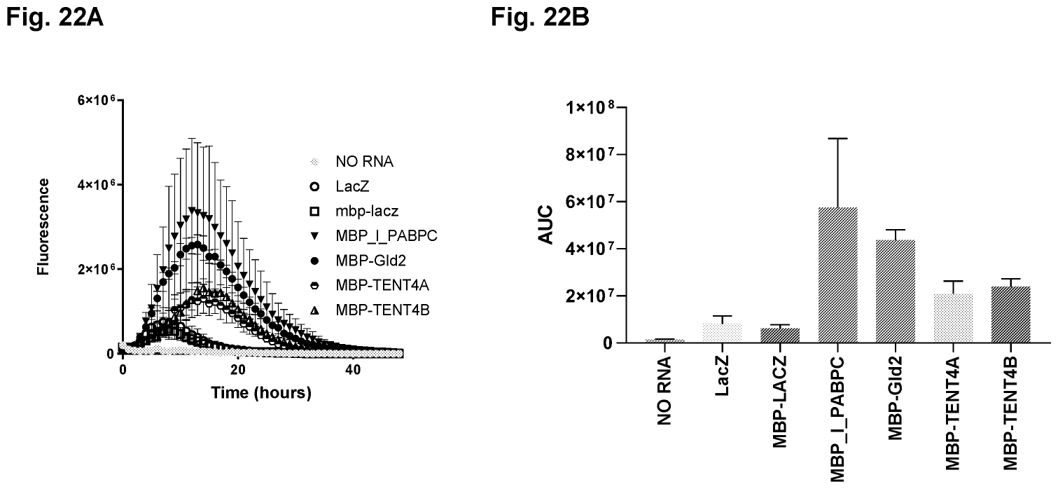

Target deg-GFP RNA was co-delivered with an mRNA encoding control protein, LacZ; or an mRNA encoding tethered control protein, MBP-LacZ; or an mRNA encoding tethered effector proteins as depicted in FIGS. 21A-21B and FIGS. 22A-22B. The experiment was done in 10× molar excess of target RNA in Hep3b.

As shown in FIGS. 21A-21B, both eIF4G1-f1 and eIF4G3-f1, and domains thereof that contained the mid domain (binds eIF4A-3) increase protein output from target RNA. As shown in FIGS. 22A-22B. PolyA binding protein and polynucleotidyl transferases such as Gld2, TENT4A and TENT4B all increase protein output from target RNA.

Taken together, this data shows that ribosome binding of eIF4G homologues via eIF3/eIF4A is important for effector function. Additionally, PABP and polynucleotidyl transferases also serve as effectors that increase protein expression and duration of expression from target RNAs.

### Other Embodiments

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims. All references described herein are incorporated by reference in their entireties.

What is claimed is:

1. A lipid nanoparticle (LNP) composition comprising:
(a) a first polynucleotide comprising: (1) a sequence encoding a therapeutic payload or a prophylactic payload, and (2) a binding element; and
(b) a second polynucleotide comprising a sequence encoding: (1) an effector molecule, and/or (2) a polypeptide that recognizes the binding element (a tether molecule),
optionally wherein, (a) and (b) each comprise an mRNA.

2. A lipid nanoparticle (LNP) composition comprising:
(a) a first polynucleotide comprising: (1) a sequence encoding a therapeutic payload or a prophylactic payload, and (2) a binding element; and
(b) a second polynucleotide comprising a sequence encoding an effector molecule,
optionally wherein, (a) and (b) each comprise an mRNA.

3. The LNP composition of claim 2, wherein the effector molecule further comprises a polypeptide that recognizes the binding element (a tether molecule).

4. The LNP composition of claim 2, wherein the effector molecule recognizes the binding element.

5. The LNP composition of any one of claims 1-4, wherein the first polynucleotide and the second polynucleotide are disposed in the same polynucleotide.

6. The LNP composition of claim 5, wherein the first polynucleotide and the second polynucleotide are separated by a protease cleavage site (e.g., a P2A, T2A, E2A, or TPE (P2A-T2A-E2A site) or an internal ribosomal entry site.

7. The LNP composition any one of claims 1-4, wherein the first polynucleotide and the second polynucleotide are disposed in different polynucleotides.

8. The LNP composition of claim 7, wherein (a) and (b) are in the same LNP.

9. The LNP composition of claim 7, wherein (a) and (b) are in different LNPs.

10. The LNP composition of any one of claims 1-4, wherein the second polynucleotide is DNA.

11. The LNP composition of claim 10, wherein the sequence encoding the effector molecule is under the control of a tissue-specific promoter.

12. The LNP composition of any one of claims 1-4, wherein expression or recruitment of the effector molecule is under the control of a trigger in a specific microenvironment or specific cell-type.

13. The LNP composition of claim 12, wherein the trigger is microRNA, receptor-mediated activation, and/or a change in pH and/or hypoxia.

14. A system comprising:
(a) a first polynucleotide comprising: (1) a sequence encoding a therapeutic payload or a prophylactic payload, and (2) a binding element; and/or
(b) a second polynucleotide comprising a sequence encoding: (1) an effector molecule, and (2) a polypeptide that recognizes the binding element (a tether molecule),
optionally wherein, (a) and (b) each comprise an mRNA.

15. A system comprising:
(a) a first polynucleotide comprising: (1) a sequence encoding a therapeutic payload or a prophylactic payload, and (2) a binding element; and/or
(b) a second polynucleotide comprising a sequence encoding an effector molecule,
optionally wherein, (a) and (b) each comprise an mRNA.

16. The system of claim 15, wherein the effector molecule further comprises a polypeptide that recognizes the binding element (a tether molecule).

17. The system of claim 15, wherein the effector molecule recognizes the binding element.

18. The system of any one of claims 14-17, wherein the system comprises (a).

19. The system of any one of claims 14-18, wherein the system comprises (b).

20. The system of any one of claims 14-19, wherein the system comprises (a) and (b).

21. The system of any one of claims 14-20, wherein the first polynucleotide and the second polynucleotide are disposed in the same polynucleotide.

22. The system of claim 21, wherein the first polynucleotide and the second polynucleotide are separated by a protease cleavage site (e.g., a P2A, T2A, E2A, or TPE (P2A-T2A-E2A site) or an internal ribosomal entry site.

23. The system of any one of claims 14-20, wherein the first polynucleotide and the second polynucleotide are disposed in different polynucleotides.

24. The system of any one of claims 14-23, wherein at least one of (a) or (b) is formulated as a lipid nanoparticle (LNP).

25. The system of claim 24, wherein (a) is formulated as an LNP.

26. The system of claim 24, wherein (b) is formulated as an LNP.

27. The system of claim 24, wherein (a) and (b) both are formulated as LNPs, e.g., the same LNP or different LNPs.

28. The system of any one of claims 14-17, wherein the second polynucleotide is DNA.

29. The system of claim 15, wherein the effector molecule is under the control of a tissue-specific promoter.

30. The system of any one of claims 14-17, wherein expression of the effector molecule or recruitment of the effector molecule is under the control of a trigger in a specific microenvironment or specific cell-type.

31. The system of claim 30, wherein the trigger is microRNA, receptor-mediated activation, and/or a change in pH and/or hypoxia.

32. A pharmaceutical composition comprising the system, or LNP composition of any one of the preceding claims.

33. A cell comprising a system, or LNP composition of any one of the preceding claims.

34. The cell of claim 33, which has been contacted with the system, or LNP composition of any of claims 1-31.

35. The cell of claim 33 or 34, which is maintained under conditions sufficient to allow for expression of one or both polynucleotides of the system, or LNP composition.

36. A method of increasing expression of a therapeutic payload or a prophylactic payload in a cell, comprising administering to the cell a system, or LNP composition of any one of claims 1-27.

37. A method of increasing expression of a therapeutic payload or a prophylactic payload, in a subject, comprising administering to the subject an effective amount of a system or LNP composition of any one of claims 1-27.

38. A method of delivering a system, or LNP composition of any one of claims 1-27, to a cell.

39. The method of claim 38, comprising contacting the cell in vitro, in vivo, or ex vivo with the system or LNP composition.

40. A method of delivering a system or LNP composition of any one of claims 1-27, to a subject having a disease or disorder, e.g., as described herein.

41. A method of modulating an immune response in a subject, comprising administering to the subject in need thereof an effective amount of a system, or LNP composition of any one of claims 1-27.

42. A method of treating, preventing, or preventing a symptom of, a disease or disorder comprising administering to a subject in need thereof an effective amount of a system, or LNP composition of any one of claims 1-27.

43. The method of any one of claims 36-42, wherein the first polynucleotide or the second polynucleotide of the system is formulated as an LNP.

44. The method of claim 43, wherein both the first and the second polynucleotides of the system are formulated as LNPs, e.g., the same or different LNPs.

45. The method of any one of claims 36-44, wherein the LNP comprising (a) and the LNP comprising (b) are administered simultaneously, e.g., substantially simultaneously.

46. The method of any one of claims 36-44, wherein the LNP comprising (a) and the LNP comprising (b) are administered sequentially.

47. The system, LNP composition, cell or method of any one of the preceding claims, wherein the tether molecule of the second polynucleotide comprises an RNA binding protein or a fragment thereof, which binds to, e.g., recognizes, the binding element of the first polynucleotide.

48. The system, LNP composition, cell or method of any one of the preceding claims, wherein the binding element of the first polynucleotide is situated upstream (5′) or downstream (3′) or in the open reading frame of the sequence encoding the therapeutic payload or a prophylactic payload.

49. The system, LNP composition, cell or method of any one of the preceding claims, wherein the binding element of the first polynucleotide is situated upstream (5′) or downstream (3′) of a 5′ UTR of the first polynucleotide.

50. The system, LNP composition, cell or method of any one of the preceding claims, wherein the binding element of the first polynucleotide is situated upstream (5′) or downstream (3′) of a 3′ UTR of the first polynucleotide.

51. The system, LNP composition, cell or method of any one of the preceding claims, wherein the binding element of the first polynucleotide is situated downstream of a 3′ UTR of the first polynucleotide.

52. The system, LNP composition, cell or method of any one of the preceding claims, wherein the binding element of the first polynucleotide is bound by the tether molecule of the second polynucleotide, e.g., a tether molecule provided in Table 1, e.g., MBP, PCP, Lambda N, U1A or PUF, 15.5 kd, LARP7 or a variant or fragment thereof.

53. The system, LNP composition, cell or method of any one of the preceding claims, wherein the binding element is chosen from a binding element provided in Table 1, e.g., MS2 (e.g., wildtype MS2, or a variant or fragment thereof).

54. The system, LNP composition, cell or method of any one of the preceding claims, wherein the binding element comprises a sequence comprising 19 nucleotides, e.g., a MS2 binding element nucleotide sequence provided in Table 2 or a sequence with at least 80%, 85%, 90%, 95%, 96%, 97%, 98%, 99% or 100% identity thereof.

55. The system, LNP composition, cell or method of any one of the preceding claims, wherein the effector molecule is chosen from: a translation factor, a splicing factor, an RNA stabilizing factor, an RNA editing factor, an RNA-binding factor, an RNA localizing factor, or a combination thereof.

56. The system, LNP composition, cell or method of any one of the preceding claims, wherein the effector molecule is a translation factor, e.g., a translation factor provided in Table 4, e.g., eIF4G; Poly A binding protein (PABP); eIF3d or a component thereof; Daz1, or a fragment, or variant or combination thereof.

57. The system, LNP composition, cell or method of any one of the preceding claims, wherein the effector molecule is a translation factor which modulates, e.g., facilitates, ribosome binding, e.g., recruitment, pre-initiation complex formation, or RNA unwinding.

58. The system, LNP composition, cell or method of any one of the preceding claims, wherein the effector molecule comprises eIF4G, e.g., wildtype eIF4G, a variant of eIF4G, or a fragment thereof.

59. The system, LNP composition, cell or method of claim 58, wherein the eIF4G variant retains ribosome binding, e.g., recruitment.

60. The system, LNP composition, cell or method of any one of the preceding claims, wherein the effector molecule comprises RNA modifying enzymes such as terminal transferases e.g., TENT4A, TENT4B, Gld2, a variant or fragment thereof.

61. The system, LNP composition, cell or method of any one of the preceding claims, wherein the effector molecule comprises an amino acid sequence provided in Table 2, or a sequence with at least 80%, 85%, 90%, 95%, 96%, 97%, 98%, 99% or 100% identity thereof.

62. The system, LNP composition, cell or method of any one of the preceding claims, wherein the effector molecule is encoded by a nucleotide sequence provided in Table 2, or a sequence with at least 80%, 85%, 90%, 95%, 96%, 97%, 98%, 99% or 100% identity thereof.

63. The system, LNP composition, cell or method of any one of the preceding claims, wherein the tether molecule binds to a binding element in the first polynucleotide.

64. The system, LNP composition, cell or method of any one of the preceding claims, wherein the tether molecule comprises a tether molecule provided in Table 1, e.g., MBP, PCP, Lambda N, U1A or PUF, 15.5 kd, LARP7 or a variant or fragment thereof.

65. The system, LNP composition, cell or method of any one of the preceding claims, wherein the tether molecule comprises MBP.

66. The system, LNP composition, cell or method of any one of the preceding claims, wherein the tether molecule comprises an amino acid sequence provided in Table 2 or a sequence with at least 80%, 85%, 90%, 95%, 96%, 97%, 98%, 99% or 100% identity thereof.

67. The system, LNP composition, cell or method of any one of the preceding claims, wherein the tether molecule is encoded by a nucleotide sequence provided in Table 2 or a sequence with at least 80%, 85%, 90%, 95%, 96%, 97%, 98%, 99% or 100% identity thereof.

68. The system, LNP composition, cell or method of any one of the preceding claims, wherein the therapeutic payload or a prophylactic payload comprises an mRNA encoding: a secreted protein, a membrane-bound protein; or an intercellular protein.

69. The system, LNP composition, cell or method of claim 65, wherein the therapeutic payload or a prophylactic payload is chosen from a cytokine, an antibody, a vaccine (e.g., an antigen, an immunogenic epitope), a receptor, an enzyme, a hormone, a transcription factor, a ligand, a membrane transporter, a structural protein, a nuclease, a growth factor, a immune modulator, or a component, variant or fragment (e.g., a biologically active fragment) thereof.

70. The system, LNP composition, cell or method of any one of the preceding claims, which results in one, two, three, four, five, six or all, or any combination thereof, of the following in a cell (e.g., in a cell contacted with the system or LNP composition):
(i) increased expression and/or level of mRNA encoding the therapeutic payload or prophylactic payload;
(ii) sustained expression and/or level of mRNA encoding the therapeutic payload or prophylactic payload;
(iii) increased expression and/or level of therapeutic payload or prophylactic payload;
(iv) sustained expression and/or level of therapeutic payload or prophylactic payload;
(v) increased stability of mRNA encoding the therapeutic payload or prophylactic payload;
(vi) increased resistance of translation of therapeutic payload or prophylactic payload to cellular environment, e.g., stress or nutrient deprivation or translation factor availability;
(vii) reduced dosing of the therapeutic payload or prophylactic payload; or
(viii) reduced toxicity, e.g., reduced modulation of a protein translated from endogenous mRNA in a cell.

71. The system, LNP composition, cell or method of any one of the preceding claims, wherein:
(a) the first polynucleotide comprises:
(1) a sequence encoding a therapeutic payload or a prophylactic payload, and
(2) a binding element comprising an MS2 sequence, e.g., 6 MS2 sequences of 19 nucleotides separated by spacers of 20 nucleotides in length;
(b) the second polynucleotide comprises a sequence encoding:
(1) an effector molecule comprising eIF4G, e.g., wildtype eIF4G, a variant or a fragment thereof; and
(2) a tether molecule comprising MBP, e.g., wildtype MBP, a variant or fragment thereof.

72. The system, LNP composition, cell or method of any one of the preceding claims, wherein the first polynucleotide comprises an mRNA comprising at least one chemical modification.

73. The system, LNP composition, cell or method of any one of the preceding claims, wherein the second polynucleotide comprises an mRNA comprising at least one chemical modification.

74. The system, LNP composition, cell or method of any one of claims 1-71, wherein the first polynucleotide comprises an mRNA which does not have any chemical modification.

75. The system, LNP composition, cell or method of any one of claims 1-71 and 74, wherein the second polynucleotide comprises an mRNA which does not have any chemical modification.

76. The system, LNP composition, cell or method of any one of claims 72-75, wherein the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl -pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

77. The system, LNP composition, cell or method of any one of the preceding claims, wherein the LNP composition comprises: (i) an ionizable lipid, e.g., an amino lipid; (ii) a sterol or other structural lipid; (iii) a non-cationic helper lipid or phospholipid; and (iv) a PEG-lipid.

78. The system, LNP composition, cell or method of claim 77, wherein the ionizable lipid comprises an amino lipid.

79. The system, LNP composition, cell or method of claim 77 or 78, wherein the ionizable lipid comprises a compound of any of Formulae (I), (IA), (IB), (II), (IIa), (IIb), (IIc), (IId), (IIe), (IIf), (IIg), (III), (IIIa1), (IIIa2), (IIIa3), (IIIa4), (IIIa5), (IIIa6), (IIIa7), or (IIIa8).

80. The system, LNP composition, cell or method of any one of claims 77-79, wherein the non-cationic helper lipid or phospholipid comprises a compound chosen from DSPC (e.g., a variant of DSPC, e.g., a compound of Formula (IV)); DPPC; or DOPC.

81. The system, LNP composition, cell or method of any one of claims 77-80, wherein the structural lipid is alpha-tocopherol, β-sitosterol or cholesterol.

82. The system, LNP composition, cell or method of any one of claims 77-81, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

83. The system, LNP composition, cell or method of any one of claims 77-82, wherein the PEG lipid is selected from the group consisting of PEG-c-DOMG, PEG-DMG, PEG-DLPE, PEG-DMPE, PEG-DPPC and PEG-DSPE lipid.

84. The system, LNP composition, cell or method of any one of claims 77-81, wherein the PEG lipid is chosen from of a compound of Formula (V), Formula (VI-A), Formula (VI-B), Formula (VI-C) or Formula (VI-D).

85. The system, LNP composition, cell or method of claim 84, wherein the PEG-lipid is a compound of Formula (VI-A).

86. The system, LNP composition, cell or method of claim 84, wherein the PEG-lipid is a compound of Formula (VI-B).

87. The system, LNP composition, cell or method of claim 84, wherein the PEG-lipid is a compound of Formula (VI-C).

88. The system, LNP composition, cell or method of claim 84, wherein the PEG-lipid is a compound of Formula (VI-D).

89. The system, LNP composition, cell or method of any one of claims 77-88, wherein the LNP comprises a molar ratio of about 20-60% ionizable lipid: 5-25% phospholipid: 25-55% cholesterol; and 0.5-15% PEG lipid.

90. The system, LNP composition, cell or method of any one of the preceding claims, which is formulated for intravenous, subcutaneous, intramuscular, intranasal, intraocular, rectal, pulmonary or oral delivery.

91. The system, LNP composition, cell or method of any one of the preceding claims, wherein the subject is a mammal, e.g., a human.


SEQUENCE LISTING (157 sequences)

SEQ ID NO: 1 - DNA, 333 bp, Artificial Sequence
  source: /note=“Description of Artificial Sequence Synthetic polynucleotide”
tgataataggctggagcctcggtggcctagcttcttgccccttgggcctccccccagcccctcctccccttcctgcacccgtacccccgtggtctttgaataaagtctgagtgggcggccaggtatgattacaacctgtgctactttagatgactattgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggag

SEQ ID NO: 2 - DNA, 333 bp, Artificial Sequence
  source: /note=“Description of Artificial Sequence Synthetic polynucleotide”
tgataatagcaggtatgattacaacctgtgctactttagatgactattgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggaggctggagcctcggtggcctagcttcttgccccttgggcctccccccagcccctcctccccttcctgcacccgtacccccgtggtctttgaataaagtctgagtgggcggc

SEQ ID NO: 3 - DNA, 223 bp, Artificial Sequence
  source: /note=“Description of Artificial Sequence Synthetic polynucleotide”
tgataatagcaggtatgattacaacctgtgctactttagatgactattgaaagaccattaggctttctaacggacgctcacgagagaggggcaccaatagggccccacggcacttcaaaggtttggcttggagtagtaacccaagcagcaacagttttgactttcggaccaccatcaggggtcccacgttgggaacacgtaactctcctactaacaagaggag

SEQ ID NO: 4 - DNA, 119 bp, Artificial Sequence
  source: /note=“Description of Artificial Sequence Synthetic polynucleotide”
tgataataggctggagcctcggtggcctagcttcttgccccttgggcctccccccagcccctcctccccttcctgcacccgtacccccgtggtctttgaataaagtctgagtgggcggc

SEQ ID NO: 5 - DNA, 57 bp, Artificial Sequence
  source: /note=“Description of Artificial Sequence Synthetic oligonucleotide”
gggaaataagagagaaaagaagagtaagaagaaatataagaccccggcgccgccacc

SEQ ID NO: 6 - protein, 117 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 7 - DNA, 351 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctac

SEQ ID NO: 8 - protein, 977 aa, Artificial Sequence
  source: /note=“Description of Artificial Sequence Synthetic polypeptide”
IFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 9 - DNA, 2931 bp, Artificial Sequence
  source: /note=“Description of Artificial Sequence Synthetic polynucleotide”
atattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 10 - protein, 1099 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 11 - DNA, 3297 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 12 - protein, 1720 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSNKAPQSTGPPPAPSPGLPQPAFPPGQTAPVVFSTPQATQMNTPSQPRQHFYPSRAQPPSSAASRVQSAAPARPGPAAHVYPAGSQVMMIPSQISYPASQGAYYIPGQGRSTYVVPTQQYPVQPGAPGFYPGASPTEFGTYAGAYYPAQGVQQFPTGVAPTPVLMNQPPQIAPKRERKTIRIRDPNQGGKDITEEIMSGARTASTPTPPQTGGGLEPQANGETPQVAVIVRPDDRSQGAIIADRPGLPGPEHSPSESQPSSPSPTPSPSPVLEPGSEPNLAVLSIPGDTMTTIQMSVEESTPISRETGEPYRLSPEPTPLAEPILEVEVTLSKPVPESEFSSSPLQAPTPLASHTVEIHEPNGMVPSEDLEPEVESSPELAPPPACPSESPVPIAPTAQPEELLNGAPSPPAVDLSPVSEPEEQAKEVTASMAPPTIPSATPATAPSATSPAQEEEMEEEEEEEEGEAGEAGEAESEKGGEELLPPESTPIPANLSQNLEAAAATQVAVSVPKRRRKIKELNKKEAVGDLLDAFKEANPAVPEVENQPPAGSNPGPESEGSGVPPRPEEADETWDSKEDKIHNAENIQPGEQKYEYKSDQWKPLNLEEKKRYDREFLLGFQFIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 13 - DNA, 5160 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcaacaaggcccctcagagcaccggccctcctcctgcccctagccctggcctgcctcagcctgccttccctcctggccagaccgcccctgtggtgttcagcacccctcaggccacccagatgaacacacctagccagcctagacagcacttctaccctagcagagcccagcctcctagcagcgccgccagcagagtgcagtccgctgcacctgccagacctggccctgccgcccacgtgtaccctgccggcagccaggtgatgatgatcccgagtcaaatcagctaccctgcaagccagggcgcctactacatcccgggccagggcagaagcacctacgtggtgcctacccagcagtaccctgtgcagcctggcgcccctggtttctatcctggcgcaagccctaccgagttcggaacttacgccggcgcttactatccagctcagggcgtgcagcagttccctaccggcgtggcccctacccctgtgctgatgaatcagccacctcagatcgcccctaagcgcgagcgcaagaccatcagaatccgcgatcctaaccagggcggcaaggacatcaccgaggagatcatgagcggcgccagaacagcaagtactccaaccccgccacaaaccggcggcggcctggagcctcaagccaacggcgagacgccacaagtggccgtgatcgtacgccctgacgaccggagccaaggtgcaatcatcgccgataggcctggcctcccaggtccggagcacagccctagcgagtcccagccgtcttcaccatcaccaacccctagtccatcccctgttctcgaaccaggcagcgagcctaacctggccgtgctgagcataccaggtgacaccatgaccaccatccagatgagcgtggaggagagcaccccaatcagcagagaaactggagagccttacagactgtccccagagccgaccccactggccgagccaatactggaggtggaggtgaccctgagcaagcctgtgcctgagagcgagttcagctcctctccactgcaggccccaactcctctcgcaagccacaccgtggagatccacgaacctaacggcatggtaccaagcgaagatcttgagccagaggtcgaatcaagcccagaactggcccctccacctgcctgcccgtctgaatctccggtccctatcgctcctacggcacagcctgaggagctgctgaacggtgccccgagccctccagcagtggacttatccccagtatcagagcctgaagaacaggccaaggaggtaactgcctctatggcgccacctaccataccttcggcaacaccggctacagcaccatctgcgactagtccggctcaggaggaggagatggaggaagaagaagaggaagaggagggcgaggccggagaggccggtgaagccgagtccgagaagggcggcgaagaacttctccctccagagtcaactcctatccctgccaaccttagtcagaatctggaggccgccgccgctactcaggttgcagtgagcgtgccaaagagacgtcgcaagatcaaggagctgaacaagaaggaggccgtgggcgacctgctggacgccttcaaggaggcaaacccggcggtgcctgaagtggagaatcagcctccggccggatcaaaccctggtcctgagagtgaaggcagcggcgtcccaccaagacctgaagaggctgacgaaacttgggacagcaaggaggacaagatccacaacgccgagaacatccagccaggcgagcagaagtacgagtacaagagcgaccagtggaagcctcttaaccttgaagagaagaagagatacgacagagagttcctgctgggcttccagttcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 14 - protein, 1099 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSIANKATPQMAQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 15 - DNA, 3297 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcgcgaacaaggcgacccctcagatggcgcagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 16 - protein, 1069 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 17 - DNA, 3207 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 18 - protein, 921 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLL

SEQ ID NO: 19 - DNA, 2763 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctc

SEQ ID NO: 20 - protein, 528 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSID

SEQ ID NO: 21 - DNA, 1584 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgac

SEQ ID NO: 22 - protein, 600 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 23 - DNA, 1800 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 24 - protein, 541 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 25 - DNA, 1623 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 26 - protein, 642 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 27 - DNA, 1926 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 28 - protein, 494 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLL

SEQ ID NO: 29 - DNA, 1482 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctc

SEQ ID NO: 30 - protein, 719 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSENSSAASASSEAGSSRSQEIEELERFIDSYVLEYQVQGLLADKTEGDGESERTQSHISQWTADCSEPLDSSCSFSRGRAPPQQNGSKDNSLDMLGTDIWAANTFDSFSGATWDLQPEKLDFTQFHRKVRHTPKQPLPHIDREGCGKGKLEDGDGINLNDIEKVLPAWQGYHPMPHEVEIAHTKKLFRRRRNDRRRQQRPPGGNKPQQHGDHQPGSAKHNRDHQKSYQGGSAPHPSGRPTHHGYSQNRRWHHGNMKHPPGDKGEAGAHRNAKETMTIENPKLEDTAGDTGHSSLEAPRSPDTLAPVASERLPPQQSGGPEVETKRKDSILPERIGERPKITLLQSSKDRLRRRLKEKDEVAVETTTPQQNKMDKLIEILNSMRNNSSDVDTKLTTFMEEAQNSTNSEEMLGEIVRTIYQKAVSDRSFAFTAAKLCDKMALFMVEGTKFRSLLLNMLQKDFTVREELQQQDVERWLGFITFLCEVFGTMRSSTGEPFRVLVCPIYTCLRELLQSQDVKEDAVLCCSMELQSTGRLLEEQLPEMMTELLASARDKMLCPSESMLTRSLLLEVIELHANSWNPLTPPITQYYNRTIQKLTA

SEQ ID NO: 31 - DNA, 2157 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgagaacagtagcgccgctagcgcgagcagcgaagccgggagcagccggtctcaggagatcgaggagctggagcggttcatcgacagctacgtgctggagtaccaggtgcagggcctgctggccgacaagaccgagggcgacggcgagagcgagcggacccagagccacatcagccagtggaccgccgactgcagcgagcccctggactctagctgctcattcagtcgtggacgggcccctccacagcagaacggcagcaaggacaacagcctggacatgctgggcaccgacatctgggccgccaacaccttcgacagcttcagcggcgccacctgggatctgcagcccgagaagctggactttacccagttccaccggaaggtgcggcacactcccaagcagcccctgccccacatcgatcgggagggctgcggcaagggcaagctggaagacggcgacggcatcaacctgaacgacatcgagaaggtgctgcctgcctggcagggctaccaccccatgccccacgaggtggagatcgcccacaccaagaagctgttccggcgacgacgcaacgaccggcgtaggcagcaacggccgcctggagggaacaagccccagcagcacggagaccaccagcccggtagcgccaagcacaaccgggaccaccagaagagctaccagggcggaagcgcaccacacccctcgggcagacccacccaccacggctacagccagaaccggcggtggcatcacggtaacatgaagcacccacccggcgacaaaggagaggccggcgctcaccgtaacgccaaggagaccatgaccatcgagaaccccaagctggaggataccgccggcgatacgggtcacagcagcctggaggcaccgcggtctcccgacaccctggcacccgtggccagcgaacggctgccaccccaacagagcggcggccctgaggttgagaccaagcggaaggacagcatcctgcccgaacggatcggtgagcggcccaagatcaccttactgcagagtagcaaggaccggctgagacggcggctgaaggagaaggacgaggtggccgtggagacaaccactccccagcagaacaagatggacaagctgatcgagatcctgaacagcatgcggaacaacagcagcgacgtggacaccaagctgaccaccttcatggaggaggcccagaacagcaccaacagcgaggagatgctgggcgagatcgtgcggaccatctaccagaaggccgtgagcgaccggagcttcgccttcaccgccgccaagctgtgcgacaagatggccctgttcatggtggagggcaccaagttccggagcttactgctgaatatgctgcagaaggacttcaccgtgcgggaggagctgcagcagcaggacgtggagcggtggctgggcttcatcaccttcctgtgcgaggtgttcggcaccatgcggagcagcaccggcgaacccttccgggtgctggtgtgccccatctacacctgcctgcgggagttgctgcagagccaggacgtgaaggaggacgccgtgctgtgctgcagcatggaactgcagagcactggccggctgctggaggagcagctgcccgagatgatgaccgagctgctcgctagcgcccgggacaagatgctgtgccccagcgagagcatgctgacccggagcctgcttctggaggtgatcgagctgcacgccaacagctggaatcccctgacccctcccatcacccagtactacaaccggaccatccagaagctgaccgcc

SEQ ID NO: 32 - protein, 323 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSLNSMRNNSSDVDTKLTTFMEEAQNSTNSEEMLGEIVRTIYQKAVSDRSFAFTAAKLCDKMALFMVEGTKFRSLLLNMLQKDFTVREELQQQDVERWLGFITFLCEVFGTMRSSTGEPFRVLVCPIYTCLRELLQSQDVKEDAVLCCSMELQSTGRLLEEQLPEMMTELLASARDKMLCPSESMLTRSLLLEVIELHANSWN

SEQ ID NO: 33 - DNA, 969 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcctgaacagcatgagaaacaacagcagcgacgtggacaccaagctgaccaccttcatggaggaggcccagaacagcaccaacagcgaggagatgctgggcgagatcgtgagaaccatctaccagaaggccgtgagcgacagaagcttcgccttcaccgccgccaagctgtgcgacaagatggccctgttcatggtggagggcaccaagttcagaagcctgctgctgaacatgctgcagaaggacttcaccgtgagagaggagctgcagcagcaggacgtggagcgatggctgggcttcatcaccttcctgtgcgaggtgttcggcaccatgagaagcagcaccggcgagcctttcagagtgctggtgtgccctatctacacctgcctgagagagctgctgcagagccaggacgtgaaggaggacgccgtgctgtgctgcagcatggagctgcagagcaccggcagactgctggaggagcagctgcctgagatgatgaccgagctgctggccagcgccagagacaagatgctgtgccctagcgagagcatgctgactagatccctactgttggaggtgatcgagctgcacgccaacagctggaac

SEQ ID NO: 34 - protein, 356 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSTTPQQNKMDKLIEILNSMRNNSSDVDTKLTTFMEEAQNSTNSEEMLGEIVRTIYQKAVSDRSFAFTAAKLCDKMALFMVEGTKFRSLLLNMLQKDFTVREELQQQDVERWLGFITFLCEVFGTMRSSTGEPFRVLVCPIYTCLRELLQSQDVKEDAVLCCSMELQSTGRLLEEQLPEMMTELLASARDKMLCPSESMLTRSLLLEVIELHANSWNPLTPPITQYYNRTIQKLTA

SEQ ID NO: 35 - DNA, 1068 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcaccacccctcagcagaacaagatggacaagctgatcgagatcctgaacagcatgagaaacaacagcagcgacgtggacaccaagctgaccaccttcatggaggaggcccagaacagcaccaacagcgaggagatgctgggcgagatcgtgagaaccatctaccagaaggccgtgagcgacagaagcttcgccttcaccgccgccaagctgtgcgacaagatggccctgttcatggtggagggcaccaagttcagaagcctgctgctgaacatgctgcagaaggacttcaccgtgagagaggagctgcagcagcaggacgtggagaggtggctgggcttcatcaccttcctgtgcgaggtgttcggcaccatgagaagcagcaccggcgagcctttcagagtgctggtgtgccctatctacacctgcctgagagagctgctgcagagccaggacgtgaaggaggacgccgtgctgtgctgcagcatggagctgcagagcaccggcagactgctggaggagcagctgcctgagatgatgaccgagctgctggccagcgccagagacaagatgctgtgccctagcgagagcatgctgacgcgcagcctcctgctggaggtgatcgagctgcacgccaacagctggaaccctctgacccctcctatcacccagtactacaacagaaccatccagaagctgaccgcc

SEQ ID NO: 36 - protein, 364 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPR

SEQ ID NO: 37 - DNA, 1092 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgccgacggcagcaagacccaggacctgttcagaagagtgagaagcatcctgaacaagctgacccctcagatgttccagcagctgatgaagcaggtgacccagctggccatcgacaccgaggagagactgaagggcgtgatcgacctgatcttcgagaaggccatcagcgagcctaacttcagcgtggcctacgccaacatgtgccggtgcctgatggccctgaaggtgcctaccaccgagaagcctaccgtgaccgtgaacttcagaaagctgctgctgaaccggtgccagaaggagttcgagaaggacaaggacgacgacgaggtgttcgagaagaagcagaaggagatggacgaggccgccaccgccgaggagagaggcagactgaaggaggagctggaggaggccagagacatcgccagaagaagaagcctgggcaacatcaagttcatcggcgagctgttcaagctgaagatgctgaccgaggccatcatgcacgactgcgtggtgaagctgctgaagaaccacgacgaggagagcctggagtgcctgtgcagactgctgaccaccatcggcaaggacctggatttcgagaaggcgaagcctagaatggaccagtacttcaaccagatggagaagatcatcaaggagaagaagaccagcagcagaatcagattcatgctgcaggacgtgctggacctgagaggcagcaactgggtgcctaga

SEQ ID NO: 38 - protein, 1168 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSSFTLTNKNVIFVAGLGGIGLDTSKELLKRDPVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAELQLSAGRYHYQLVWCQK

SEQ ID NO: 39 - DNA, 3504 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagctcgttcacgctaacgaacaagaacgtcatcttcgtagcgggacttggcggtatcggcctagacacgtcgaaggaactactaaagcgtgacccggtagtcctccaacgtcgcgattgggagaacccgggcgtaacgcaactaaaccgtcttgcggcgcacccgccgtttgcgtcgtggcgtaactcggaggaggcgcgaacggatcgtccgtcgcaacaactacgttcgctcaacggggagtggcgcttcgcgtggttcccggcgccggaggcggtaccggagtcgtggctcgagtgcgatctaccggaggcggacacggtcgtcgtaccgtcgaactggcaaatgcacggttacgacgcgccgatatacacgaacgtcacgtacccgataacggtaaacccgccgttcgtcccgacggagaacccgacggggtgctactcgctaacgttcaacgttgacgagtcgtggttgcaagagggtcaaacgcgtatcatattcgacggtgtaaactcggcgttccacctgtggtgcaacgggcgctgggtagggtacggccaagactcgcgtctaccgtcggagttcgacctatcggcgttcctacgagcgggtgagaaccggctagcggtcatggtcctacgttggtcggacggttcgtacctcgaggaccaagacatgtggcgaatgtcgggtatcttccgcgacgtatcgctcctacacaagccgacgacgcaaatctcggacttccacgtcgcgacgcgtttcaacgacgatttctcgcgggcagtcctagaggcggaggtccaaatgtgcggggagctacgtgactacctccgtgtcacggtatcgctctggcaaggtgagacgcaagtagcgtcgggtacggcgccgttcggcggtgagatcatcgacgagcgtggtgggtacgcggaccgtgtaacgctacgtctaaacgtcgagaacccgaagctctggtcggcggagatcccgaacctataccgtgcggtcgtcgagctacatacggcggacgggacgctaatagaggcggaagcgtgcgacgtcgggtttcgagaggttcgtatagagaacgggctgctacttctaaacgggaagccgttgctcatacgtggtgtcaaccgtcacgagcaccacccgctacacggtcaagtaatggacgagcaaacgatggtacaagacatcctactaatgaagcagaacaacttcaacgcggtacgctgttcgcattacccgaaccatccgttgtggtacacgctttgcgaccgatacggtctatacgtcgtagacgaggcgaacatagagacgcacgggatggtaccgatgaatcgcctaacggacgacccgcgttggctaccggcgatgtcggagcgagtcacgcgtatggtccaacgggaccgtaaccacccgtcggtaataatctggtcgctaggcaacgaatcggggcacggggcgaaccacgacgcgctataccgttggatcaagtcggtagacccgtcgcgtccggtacaatacgaaggtggcggtgcggacacgacggcgacggacatcatctgcccgatgtacgcgcgcgtcgacgaagaccaaccgttcccggcggtaccgaagtggtcgatcaagaagtggctctcgttgccgggtgaaacgcgtccgttgatactttgcgagtacgcgcacgcgatgggcaactcgttgggtgggttcgcgaagtactggcaggcgttccgtcaatacccgcgtctacagggtgggttcgtctgggactgggtagaccaatcgctaatcaagtacgacgagaacggcaacccgtggtcggcgtacggtggggacttcggggacacgccgaacgaccgccaattctgtatgaacggcctagtcttcgcggaccgaacgccgcacccggcgttgacggaggcgaagcatcaacaacaattcttccaattccgtctatcggggcaaacgatcgaggtaacgtcggagtacttgttccggcactcggacaacgagctactacactggatggtagcactagacggcaagccgctagcgtcgggagaagtccctttggacgtcgcgccgcaaggtaagcaactaatcgagctaccggagctaccgcaaccggagtcggcgggtcaactgtggttgacggtccgtgtcgttcaaccgaacgcgacggcgtggtcggaggcgggtcacatctcggcgtggcagcagtggcgtctagcggagaacctctcggtcacgctaccggcggcgtcgcacgcgataccgcatctaacgacgtcggagatggacttctgcatcgagttggggaacaagaggtggcagttcaaccgtcaatcgggattcctatcgcaaatgtggataggtgacaagaagcaactactaacgccgctacgtgatcagttcacgcgtgctccgctagacaacgacataggtgtttcggaggcgacgcgtatagacccgaacgcgtgggtggagcggtggaaggcggcggggcactaccaagcggaggcggcgctactacagtgcacggcggacacgctagcggacgcggtattgatcacgacggcgcacgcgtggcaacaccaggggaagacgctattcatctcgcgtaagacgtaccgtatcgacggttcgggccaaatggcgatcacggtcgacgtagaggtagcgtcggacacgccgcatccggcgcgcatcggtctaaactgccaactagcgcaagtagcggagcgtgtaaactggctagggctagggccgcaagagaactatccggaccgcctaacggcggcgtgcttcgaccgttgggacctaccgctttcggacatgtataccccgtacgtcttcccgtcggagaacgggttgaggtgcgggacgcgcgagctaaactacgggccgcaccagtggcgaggggacttccaattcaacatatcgcgttactcgcaacaacaactaatggagacgtcgcaccgtcacctactacacgcggaggaggggacgtggctaaacatcgacgggttccacatgggcataggtggggacgactcgtggtcgccgtcggtctcggcggagctccaactctcggcgggtcgttaccattaccaactagtttggtgccagaag

SEQ ID NO: 40 - protein, 478 aa, Artificial Sequence
  source: /note=“Description of Artificial Sequence Synthetic polypeptide”
DPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 41 - DNA, 1434 bp, Artificial Sequence
  source: /note=“Description of Artificial Sequence Synthetic polynucleotide”
gaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 42 - protein, 977 aa, Artificial Sequence; identical to SEQ ID NO: 8
  source: /note=“Description of Artificial Sequence Synthetic polypeptide”

SEQ ID NO: 43 - DNA, 2931 bp, Artificial Sequence; identical to SEQ ID NO: 9
  source: /note=“Description of Artificial Sequence Synthetic polynucleotide”

SEQ ID NO: 44 - protein, 977 aa, Artificial Sequence
  source: /note=“Description of Artificial Sequence Synthetic polypeptide”
IFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSIANKATPQMAQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAVPTESTDNRRVVQRSSLSRERGEKAGDRGDRLERSERGGDRGDRLDRARTPATKRSFSKEVEERSRERPSQPEGLRKAASLTEDRDRGRDAVKREAALPPVSPLKAALSEEELEKKSKAIIEEYLHLNDMKEAVQCVQELASPSLLFIFVRHGVESTLERSAIAREHMGQLLHQLLCAGHLSTAQYYQGLYEILELAEDMEIDIPHVWLYLAELVTPILQEGGVPMGELFREITKPLRPLGKAASLLLEILGLLCKSMGPKKVGTLWREAGLSWKEFLPEGQDIGAFVAEQKVEYTLGEESEAPGQRALPSEELNRQLEKLLKEGSSNQRVFDWIEANLSEQQIVSNTLVRALMTAVCYSAIIFETPLRVDVAVLKARAKLLQKYLCDEQKELQALYALQALVVTLEQPPNLLRMFFDALYDEDVVKEDAFYSWESSKDPAEQQGKGVALKSVTAFFKWLREAEEESDHN

SEQ ID NO: 45 - DNA, 2931 bp, Artificial Sequence
  source: /note=“Description of Artificial Sequence Synthetic polynucleotide”
atattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcgcgaacaaggcgacccctcagatggcgcagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtgccgaccgagagcaccgacaaccggcgagttgtgcagagaagcagcctgagcagagagaggggcgagaaagccggcgatagaggtgaccgactggagagaagcgagagaggaggtgatagaggcgaccggcttgacagagccagaacccctgccacaaagcgatcgttcagcaaggaggtcgaagagaggtccagggagcgccctagccagccggaaggactcagaaaggcagccagtctaacagaggaccgcgacaggggaagagacgccgtgaagagggaggccgcactgcctcctgtgagccctctgaaggcggcactgagcgaagaagaacttgagaagaagagtaaggcgatcatcgaggagtacctgcacctgaacgacatgaaggaggccgtgcagtgcgtgcaagagctcgcgagcccatcactgctgttcatcttcgtccggcacggcgtggagtccacactggaaagatctgccattgctagggagcatatgggccagttgttgcaccaattgctttgcgccggccacctaagtactgcccagtactatcagggtttatacgagatcctcgaactggccgaagacatggagatcgacatccctcacgtgtggctgtacctcgcagaactggtgacccctatcctgcaggagggcggcgttccaatgggtgagttgttcagagaaatcacaaagccactgcgcccactgggcaaggcggctagtctccttctggagattctcggcctgctctgtaagagcatgggtccaaagaaggtgggcaccctgtggagggaagctggactctcctggaaggaattcctccctgagggtcaggacatcggcgccttcgtggccgaacagaaggttgagtacaccctgggagaggaatcggaagcgccgggtcagcgggctctgccgagtgaggagctcaacagacaactcgagaagctcctcaaggagggatcctcaaatcagagagtgttcgactggatcgaggccaatctcagcgagcagcaaatcgtgagcaacacgttggttcgcgctctcatgacagccgtgtgctactcagccattatcttcgagacgcctctccgcgtggacgtggcagtgctcaaggcccgcgctaagctgttacagaagtacctgtgcgacgagcagaaggaactgcaggccctgtacgccctgcaagctctggtcgtcacactcgagcagcctcctaacctgctgagaatgttcttcgacgcattgtacgacgaggacgttgtgaaggaagacgctttctacagctgggagtctagtaaggatcctgcggaacaacaaggcaagggcgttgctctaaagagcgtgaccgccttcttcaagtggctgcgagaggctgaggaagagtcggaccacaac

SEQ ID NO: 46 - protein, 478 aa, Artificial Sequence
  source: /note=“Description of Artificial Sequence Synthetic polypeptide”
DPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSIANKATPQMAQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 47 - DNA, 1434 bp, Artificial Sequence
  source: /note=“Description of Artificial Sequence Synthetic polynucleotide”
gaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcgccaacaaggcgacccctcagatggcgcagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 48 - protein, 635 aa, Artificial Sequence
  source: /note=“Description of Artificial Sequence Synthetic polypeptide”
NPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTV

SEQ ID NO: 49 - DNA, 1905 bp, Artificial Sequence
  source: /note=“Description of Artificial Sequence Synthetic polynucleotide”
aaccccagcgccccaagctaccccatggccagcctgtacgtgggcgacctgcaccccgacgttaccgaggccatgctgtacgagaaattttctcccgctggccccatcctgagcatccgggtgtgccgggacatgattacccggcgctctctggggtacgcctacgtgaacttccagcagcccgcagacgccgaacgggccctggacaccatgaacttcgacgtgatcaagggcaagcccgtgcggatcatgtggagccagcgggacccaagcctgagaaagagcggcgtgggcaacatcttcatcaagaatctggacaagagcatcgacaacaaggccctgtacgacaccttcagcgccttcggcaatatcttaagctgcaaggtggtgtgcgacgagaacggcagcaagggctacggcttcgtgcacttcgaaacacaagaagccgcagagcgggccatcgagaagatgaacggcatgctgctgaacgaccggaaggtgttcgtgggccggttcaagagccggaaggagcgggaggctgaactcggtgcccgggccaaggagttcaccaacgtgtacatcaagaacttcggcgaggacatggacgacgagcggttgaaggacctgttcggcaagttcggccccgccctgtctgtgaaggtgatgaccgacgagagcggcaagtcgaagggtttcggctttgtgagcttcgagcggcacgaggacgcacagaaggccgtggacgaaatgaacggtaaggagcttaacggtaagcagatctacgttggccgggcccagaagaaggtggagcggcagaccgagctgaagcggaagttcgagcagatgaagcaggaccggatcacccggtaccagggcgtgaacctatacgtgaagaacctggacgacggcatcgacgacgaacggctgcggaaggaattcagccccttcggcaccatcaccagcgccaaggtaatgatggagggtggccggtcgaagggattcggtttcgtgtgcttcagcagccccgaggaggccaccaaggccgttactgagatgaacggccggatcgtagccacaaagccactttacgtcgccctggcccaacgcaaggaggaacggcaggcccacctgaccaaccagtacatgcagcggatggccagcgtgcgggcagtgcccaaccccgtgatcaacccctaccaacccgcaccaccaagcggctacttcatggccgccatccctcagacccagaaccgggccgcctactacccacccagccagatcgcccagttgcggcctagtccccgctggacggctcaaggagcacgaccccaccccttccagaacatgcccggagccatcagaccagctgcccctaggccacccttcagcaccatgcggcccgcctcaagccaggtgccccgggtgatgagcacccagcgggtggccaacaccagcacccagaccatgggccctagaccagcagctgctgcggctgctgccactcctgccgtccggaccgtgccacagtacaagtacgccgccggcgttcggaacccacagcagcacctgaacgcccagccccaggtgaccatgcagcagcctgccgtgcacgtgcagggccaggagcccctgaccgccagcatgttagcaagcgcccctccacaggagcagaagcagatgctgggcgaaaggctgtttcccctgatccaagcgatgcatcccaccctggccggcaagatcaccggtatgctgctggagatcgataatagcgagctgctgcacatgctggagagccccgagagtcttagatctaaggtggacgaggccgtggccgtgctccaggctcaccaggcaaaggaggccgcccagaaagccgtgaactccgccacaggcgtgcccaccgtg

SEQ ID NO: 50 - protein, 757 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTV

SEQ ID NO: 51 - DNA, 2271 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcaaccccagcgccccaagctaccccatggccagcctgtacgtgggcgacctgcaccccgacgttaccgaggccatgctgtacgagaaattttctcccgctggccccatcctgagcatccgggtgtgccgggacatgattacccggcgctctctggggtacgcctacgtgaacttccagcagcccgcagacgccgaacgggccctggacaccatgaacttcgacgtgatcaagggcaagcccgtgcggatcatgtggagccagcgggacccaagcctgagaaagagcggcgtgggcaacatcttcatcaagaatctggacaagagcatcgacaacaaggccctgtacgacaccttcagcgccttcggcaatatcttaagctgcaaggtggtgtgcgacgagaacggcagcaagggctacggcttcgtgcacttcgaaacacaagaagccgcagagcgggccatcgagaagatgaacggcatgctgctgaacgaccggaaggtgttcgtgggccggttcaagagccggaaggagcgggaggctgaactcggtgcccgggccaaggagttcaccaacgtgtacatcaagaacttcggcgaggacatggacgacgagcggttgaaggacctgttcggcaagttcggccccgccctgtctgtgaaggtgatgaccgacgagagcggcaagtcgaagggtttcggctttgtgagcttcgagcggcacgaggacgcacagaaggccgtggacgaaatgaacggtaaggagcttaacggtaagcagatctacgttggccgggcccagaagaaggtggagcggcagaccgagctgaagcggaagttcgagcagatgaagcaggaccggatcacccggtaccagggcgtgaacctatacgtgaagaacctggacgacggcatcgacgacgaacggctgcggaaggaattcagccccttcggcaccatcaccagcgccaaggtaatgatggagggtggccggtcgaagggattcggtttcgtgtgcttcagcagccccgaggaggccaccaaggccgttactgagatgaacggccggatcgtagccacaaagccactttacgtcgccctggcccaacgcaaggaggaacggcaggcccacctgaccaaccagtacatgcagcggatggccagcgtgcgggcagtgcccaaccccgtgatcaacccctaccaacccgcaccaccaagcggctacttcatggccgccatccctcagacccagaaccgggccgcctactacccacccagccagatcgcccagttgcggcctagtccccgctggacggctcaaggagcacgaccccaccccttccagaacatgcccggagccatcagaccagctgcccctaggccacccttcagcaccatgcggcccgcctcaagccaggtgccccgggtgatgagcacccagcgggtggccaacaccagcacccagaccatgggccctagaccagcagctgctgcggctgctgccactcctgccgtccggaccgtgccacagtacaagtacgccgccggcgttcggaacccacagcagcacctgaacgcccagccccaggtgaccatgcagcagcctgccgtgcacgtgcagggccaggagcccctgaccgccagcatgttagcaagcgcccctccacaggagcagaagcagatgctgggcgaaaggctgtttcccctgatccaagcgatgcatcccaccctggccggcaagatcaccggtatgctgctggagatcgataatagcgagctgctgcacatgctggagagccccgagagtcttagatctaaggtggacgaggccgtggccgtgctccaggctcaccaggcaaaggaggccgcccagaaagccgtgaactccgccacaggcgtgcccaccgtg

SEQ ID NO: 52 - protein, 913 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPRVAWIQPEQKGPANALWMQIWETSQGVGRGGSGFASYFCLNSPALDTAAAAGAAGRGSGGLGPALPAASPPPPGPTAPAALPPALLTALGPAAEGARRLHKSPSLSSSSSSSSSNAESGTESPGCSSSSSSSASLGRPGGGRGGAFFNFADGAPSAPGTANGHPGPRGPAPAGSPSQHQFHPGRRKRENKASTYGLNYLLSGSRAAALSGGGGPGAQAPRPGTPWKSRAYSPGIQGLHEEIIDFYNFMSPCPEEAAMRREVVKRIETVVKDLWPTADVQIFGSFSTGLYLPTSDIDLVVFGKWERPPLQLLEQALRKHNVAEPCSIKVLDKATVPIIKLTDQETEVKVDISFNMETGVRAAEFIKNYMKKYSLLPYLILVLKQFLLQRDLNEVFTGGISSYSLILMAISFLQLHPRIDARRADENLGMLLVEFFELYGRNFNYLKTGIRIKEGGAYIAKEEIMKAMTSGYRPSMLCIEDPLLPGNDVGRSSYGAMQVKQVFDYAYIVLSHAVSPLARSYPNRDAESTLGRIIKVTQEVIDYRRWIKEKWGSKAHPSPGMDSRIKIKERIATCNGEQTQNREPESPYGQRLTLSLSSPQLLSSGSSASSVSSLSGSDVDSDTPPCTTPSVYQFSLQAPAPLMAGLPTALPMPSGKPQPTTSRTLIMTTNNQTRFTIPPPTLGVAPVPCRQAGVEGTASLKAVHHMSSPAIPSASPNPLSSPHLYHKQHNGMKLSMKGSHGHTQGGGYSSVGSGGVRPPVGNRGHHQYNRTGWRRKKHTHTRDSLPVSLSR

SEQ ID NO: 53 - DNA, 2373 bp, Artificial Sequence
  source: /note=“Description of Artificial Sequence Synthetic polynucleotide”
gatccccgggtggcctggatccagcccgagcagaagggccccgccaacgcactgtggatgcagatctgggagacctctcagggcgtagggaggggaggaagcggcttcgccagctacttctgcctgaacagccccgctctggatacggcagccgcagcaggagccgctgggagaggctctggaggcctgggaccagcactgcccgctgcttcaccaccacctccaggccctaccgcacccgcagcccttccacccgccctgctgaccgctctgggcccagctgcagagggtgctcggcggctgcacaagagccccagcctgagcagcagcagtagcagctccagcagcaacgccgagagcggcacagagagccccggctgcagcagcagcagctccagttccgcaagcctcggcagaccaggcggtggcagaggcggtgcattcttcaacttcgctgacggagcgccaagcgctccagggacagccaacggacacccaggcccaagaggtccggcacccgctggaagccccagccagcaccagttccaccccggcagacggaagcgggagaacaaggccagcacctacggcctgaactacctgctgagcggctctcgagccgctgccctctctggtggcggtggccctggtgctcaggccccaagacctggcaccccgtggaagtcacgggcctacagccccggtatccagggcctgcacgaggagatcatcgacttctacaacttcatgagcccctgtcccgaggaagccgccatgcggagggaggtggtgaagcggatcgagaccgtggtgaaggacctgtggcccaccgcagacgtgcagatcttcggcagcttcagcaccggcctgtacctgcccaccagcgacatcgacctggtggtgttcggcaagtgggaacgccctcccctgcagctgctggagcaggccctgcggaagcacaacgtggccgagccctgcagcatcaaggtgctggacaaggccaccgtgcccatcatcaagctgaccgaccaggagaccgaggtgaaggtggacatcagcttcaacatggagaccggcgtgcgtgcagcagagttcatcaagaactacatgaagaagtacagcctgctgccctacctgatcctggtgctgaagcagttcctgctgcagcgggacctgaacgaggtgttcaccggcggcatcagcagctacagcctgatcctcatggccatcagcttcctgcagctgcacccgcggatcgacgcaaggcgggccgacgagaacctgggcatgctgctggtggagttcttcgagctgtacggccggaacttcaactacctgaagaccggcatccggatcaaggagggcggcgcctacatcgccaaggaggagatcatgaaggccatgaccagcggctaccggcccagcatgctgtgcatcgaggaccctctgctgcccggcaacgacgtgggccggagcagctacggcgccatgcaggtgaagcaggtgttcgactacgcctacatcgtgctgagccacgccgtgagcccactggcccggagctaccccaaccgggacgccgagagcaccctgggccggatcatcaaggtgacccaggaggtgatcgactaccggcggtggatcaaggagaagtggggcagcaaggcccacccatctcccggcatggacagccggatcaagatcaaggagcggatcgccacctgcaacggcgagcagacccagaaccgggagcccgaaagcccctacggccagcgtctgaccctgagcctgagctctccccagctgctgagcagcggcagcagcgccagcagcgtgagcagcctgagcggcagcgacgtggacagcgacaccccaccctgcaccaccccaagcgtgtaccagttctcactgcaggcccctgctccgctgatggctggcctgcctaccgccctgcccatgcccagcggcaagccccagcccaccaccagccggaccctgatcatgaccaccaacaaccagacccggttcaccatcccaccaccaaccctgggcgtggctcctgtgccttgtcggcaggccggagtggagggcaccgccagcctgaaggccgtgcaccacatgagcagcccagccatccccagcgccagccccaaccctctgagcagcccacacctgtaccacaagcagcacaacggcatgaagctgagcatgaagggcagccacggacacacccagggcggagggtactcaagcgttgggagcggaggggtacggcctccagtgggcaaccggggccaccaccagtacaacaggaccggctggagacggaagaagcacacccacacccgggactctctgcccgtgagcctgagccgg

SEQ ID NO: 54 - protein, 791 aa, Artificial Sequence
  source: /note=“Description of Artificial Sequence Synthetic polypeptide”
DPRVAWIQPEQKGPANALWMQIWETSQGVGRGGSGFASYFCLNSPALDTAAAAGAAGRGSGGLGPALPAASPPPPGPTAPAALPPALLTALGPAAEGARRLHKSPSLSSSSSSSSSNAESGTESPGCSSSSSSSASLGRPGGGRGGAFFNFADGAPSAPGTANGHPGPRGPAPAGSPSQHQFHPGRRKRENKASTYGLNYLLSGSRAAALSGGGGPGAQAPRPGTPWKSRAYSPGIQGLHEEIIDFYNFMSPCPEEAAMRREVVKRIETVVKDLWPTADVQIFGSFSTGLYLPTSDIDLVVFGKWERPPLQLLEQALRKHNVAEPCSIKVLDKATVPIIKLTDQETEVKVDISFNMETGVRAAEFIKNYMKKYSLLPYLILVLKQFLLQRDLNEVFTGGISSYSLILMAISFLQLHPRIDARRADENLGMLLVEFFELYGRNFNYLKTGIRIKEGGAYIAKEEIMKAMTSGYRPSMLCIEDPLLPGNDVGRSSYGAMQVKQVFDYAYIVLSHAVSPLARSYPNRDAESTLGRIIKVTQEVIDYRRWIKEKWGSKAHPSPGMDSRIKIKERIATCNGEQTQNREPESPYGQRLTLSLSSPQLLSSGSSASSVSSLSGSDVDSDTPPCTTPSVYQFSLQAPAPLMAGLPTALPMPSGKPQPTTSRTLIMTTNNQTRFTIPPPTLGVAPVPCRQAGVEGTASLKAVHHMSSPAIPSASPNPLSSPHLYHKQHNGMKLSMKGSHGHTQGGGYSSVGSGGVRPPVGNRGHHQYNRTGWRRKKHTHTRDSLPVSLSR

SEQ ID NO: 55 - DNA, 2739 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgatccccgggtggcctggatccagcccgagcagaagggccccgccaacgcactgtggatgcagatctgggagacctctcagggcgtagggaggggaggaagcggcttcgccagctacttctgcctgaacagccccgctctggatacggcagccgcagcaggagccgctgggagaggctctggaggcctgggaccagcactgcccgctgcttcaccaccacctccaggccctaccgcacccgcagcccttccacccgccctgctgaccgctctgggcccagctgcagagggtgctcggcggctgcacaagagccccagcctgagcagcagcagtagcagctccagcagcaacgccgagagcggcacagagagccccggctgcagcagcagcagctccagttccgcaagcctcggcagaccaggcggtggcagaggcggtgcattcttcaacttcgctgacggagcgccaagcgctccagggacagccaacggacacccaggcccaagaggtccggcacccgctggaagccccagccagcaccagttccaccccggcagacggaagcgggagaacaaggccagcacctacggcctgaactacctgctgagcggctctcgagccgctgccctctctggtggcggtggccctggtgctcaggccccaagacctggcaccccgtggaagtcacgggcctacagccccggtatccagggcctgcacgaggagatcatcgacttctacaacttcatgagcccctgtcccgaggaagccgccatgcggagggaggtggtgaagcggatcgagaccgtggtgaaggacctgtggcccaccgcagacgtgcagatcttcggcagcttcagcaccggcctgtacctgcccaccagcgacatcgacctggtggtgttcggcaagtgggaacgccctcccctgcagctgctggagcaggccctgcggaagcacaacgtggccgagccctgcagcatcaaggtgctggacaaggccaccgtgcccatcatcaagctgaccgaccaggagaccgaggtgaaggtggacatcagcttcaacatggagaccggcgtgcgtgcagcagagttcatcaagaactacatgaagaagtacagcctgctgccctacctgatcctggtgctgaagcagttcctgctgcagcgggacctgaacgaggtgttcaccggcggcatcagcagctacagcctgatcctcatggccatcagcttcctgcagctgcacccgcggatcgacgcaaggcgggccgacgagaacctgggcatgctgctggtggagttcttcgagctgtacggccggaacttcaactacctgaagaccggcatccggatcaaggagggcggcgcctacatcgccaaggaggagatcatgaaggccatgaccagcggctaccggcccagcatgctgtgcatcgaggaccctctgctgcccggcaacgacgtgggccggagcagctacggcgccatgcaggtgaagcaggtgttcgactacgcctacatcgtgctgagccacgccgtgagcccactggcccggagctaccccaaccgggacgccgagagcaccctgggccggatcatcaaggtgacccaggaggtgatcgactaccggcggtggatcaaggagaagtggggcagcaaggcccacccatctcccggcatggacagccggatcaagatcaaggagcggatcgccacctgcaacggcgagcagacccagaaccgggagcccgaaagcccctacggccagcgtctgaccctgagcctgagctctccccagctgctgagcagcggcagcagcgccagcagcgtgagcagcctgagcggcagcgacgtggacagcgacaccccaccctgcaccaccccaagcgtgtaccagttctcactgcaggcccctgctccgctgatggctggcctgcctaccgccctgcccatgcccagcggcaagccccagcccaccaccagccggaccctgatcatgaccaccaacaaccagacccggttcaccatcccaccaccaaccctgggcgtggctcctgtgccttgtcggcaggccggagtggagggcaccgccagcctgaaggccgtgcaccacatgagcagcccagccatccccagcgccagccccaaccctctgagcagcccacacctgtaccacaagcagcacaacggcatgaagctgagcatgaagggcagccacggacacacccagggcggagggtactcaagcgttgggagcggaggggtacggcctccagtgggcaaccggggccaccaccagtacaacaggaccggctggagacggaagaagcacacccacacccgggactctctgcccgtgagcctgagccgg

SEQ ID NO: 56 - protein, 665 aa, Artificial Sequence
  source: /note=“Description of Artificial Sequence Synthetic polypeptide”
DPRIAWFQPEQLGPSNSLWMQIWETTQGLRNLYFNHHCHSSGGASGGGGSSSSSSTATGGSGSSTGSPGGAASAPAPAPAGMYRSGERLLGSHALPAEQRDFLPLETTNNNNNHHQPGAWARRAGSSASSPPSASSSPHPSAAVPAADPADSASGSSNKRKRDNKASTYGLNYSLLQPSGGRAAGGGRADGGGVVYSGTPWKRRNYNQGVVGLHEEISDFYEYMSPRPEEEKMRMEVVNRIESVIKELWPSADVQIFGSFKTGLYLPTSDIDLVVFGKWENLPLWTLEEALRKHKVADEDSVKVLDKATVPIIKLTDSFTEVKVDISFNVQNGVRAADLIKDFTKKYPVLPYLVLVLKQFLLQRDLNEVFTGGIGSYSLFLMAVSFLQLHPREDACIPNTNYGVLLIEFFELYGRHFNYLKTGIRIKDGGSYVAKDEVQKNMLDGYRPSMLYIEDPLQPGNDVGRSSYGAMQVKQAFDYAYVVLSHAVSPIAKYYPNNETESILGRIIRVTDEVATYRDWISKQWGLKNRPEPSCNGPVSSSSATQSSSSDVDSDATPCKTPKQLLCRPSTGNRVGSQDVSLESSQAVGKMQSTQTTNTSNSTNKSQHGSARLFRSSSKGFQGTTQTSHGSLMTNKQHQGKSNNQYYHGKKRKHKRDAPLSDLCR

SEQ ID NO: 57 - DNA, 1995 bp, Artificial Sequence
  source: /note=“Description of Artificial Sequence Synthetic polynucleotide”
gatccccggatcgcctggttccagcccgagcagctaggcccaagcaacagcctgtggatgcagatctgggagaccacccagggcctgcggaacctgtacttcaaccaccactgtcacagctctggcggcgcaagtggcggaggcggcagtagcagcagctcttcaacagcaaccggaggcagcggctcgtccaccggcagtccgggtggagccgccagtgccccagctcctgccccagcaggcatgtaccggagcggcgaacggctgctgggcagccacgctctgcctgccgagcagcgggacttcctgcccctggagaccaccaataacaacaacaatcaccaccaacccggggcttgggctcgacgagccggctccagtgcaagcagtccacctagcgccagcagctctccacaccccagcgccgctgttccagcagccgatcccgccgacagtgccagcggcagcagcaacaagcggaagcgggacaacaaggccagcacctacggcctgaactacagcctgctgcaaccttctggaggccgtgcagctggcggaggtagagccgacggtggcggtgtggtgtacagcggcacgccctggaagcggcggaactacaaccagggcgtggtgggcctgcacgaggagatcagcgacttctacgagtacatgagcccacggcccgaagaggagaagatgcggatggaggtggtgaaccggatcgagagcgtgatcaaggagctgtggcctagcgccgacgtgcagatcttcggcagcttcaagaccggcctgtacctgcccaccagcgacatcgacctggtggtgttcggcaagtgggagaacctgcccctgtggaccctggaggaggccctgcggaaacacaaggtggccgacgaggacagcgtgaaggtgctggacaaggccaccgtgcccatcatcaagctgaccgacagcttcaccgaggtgaaggtggacatcagcttcaacgtgcagaacggcgtgagggcagccgacctgatcaaggacttcaccaagaagtaccccgtgctgccctacctggtgctggtgctgaagcagttcctgctgcagcgggacctgaacgaggtgttcaccggcggcatcggcagctacagcctgttcctgatggccgtgagcttcctgcagctgcatccccgtgaggacgcctgcatccccaacaccaactacggcgtgctgctgatcgagttcttcgagctgtacggccggcacttcaactacctgaagaccggcatccggatcaaggacggcggcagctacgtggccaaggacgaggtgcagaagaacatgctggacggctaccggcccagcatgctgtacatcgaggacccactgcagcccggcaacgacgtgggccggagcagctacggcgccatgcaggtgaagcaggccttcgactacgcctacgtggtgctgagccacgccgtgagccccatcgccaagtactaccccaacaacgagaccgagagcatcctgggccggatcatccgggtgaccgacgaggtggccacctaccgggactggatcagcaagcagtggggcctgaagaaccggcccgagcctagctgcaacggccccgtgagctcaagcagcgccacccagagcagcagcagcgacgtggacagcgacgccactccctgcaagacccctaagcagctgctgtgccgtcccagcaccggcaaccgggtgggcagccaggacgtgagcctggagagcagccaggccgtgggcaagatgcagagcacccagaccaccaacaccagcaacagcaccaacaagagccaacacggcagcgcacggctgttccggagcagcagcaagggcttccagggcaccacccagaccagccacggcagcctgatgaccaacaagcagcaccagggcaagagcaacaaccagtactaccacggcaagaagcggaagcacaagcgggacgctcccctgagcgacctgtgccgg

SEQ ID NO: 58 - protein, 787 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPRIAWFQPEQLGPSNSLWMQIWETTQGLRNLYFNHHCHSSGGASGGGGSSSSSSTATGGSGSSTGSPGGAASAPAPAPAGMYRSGERLLGSHALPAEQRDFLPLETTNNNNNHHQPGAWARRAGSSASSPPSASSSPHPSAAVPAADPADSASGSSNKRKRDNKASTYGLNYSLLQPSGGRAAGGGRADGGGVVYSGTPWKRRNYNQGVVGLHEEISDFYEYMSPRPEEEKMRMEVVNRIESVIKELWPSADVQIFGSFKTGLYLPTSDIDLVVFGKWENLPLWTLEEALRKHKVADEDSVKVLDKATVPIIKLTDSFTEVKVDISFNVQNGVRAADLIKDFTKKYPVLPYLVLVLKQFLLQRDLNEVFTGGIGSYSLFLMAVSFLQLHPREDACIPNTNYGVLLIEFFELYGRHFNYLKTGIRIKDGGSYVAKDEVQKNMLDGYRPSMLYIEDPLQPGNDVGRSSYGAMQVKQAFDYAYVVLSHAVSPIAKYYPNNETESILGRIIRVTDEVATYRDWISKQWGLKNRPEPSCNGPVSSSSATQSSSSDVDSDATPCKTPKQLLCRPSTGNRVGSQDVSLESSQAVGKMQSTQTTNTSNSTNKSQHGSARLFRSSSKGFQGTTQTSHGSLMTNKQHQGKSNNQYYHGKKRKHKRDAPLSDLCR

SEQ ID NO: 59 - DNA, 2361 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgatccccggatcgcctggttccagcccgagcagctaggcccaagcaacagcctgtggatgcagatctgggagaccacccagggcctgcggaacctgtacttcaaccaccactgtcacagctctggcggcgcaagtggcggaggcggcagtagcagcagctcttcaacagcaaccggaggcagcggctcgtccaccggcagtccgggtggagccgccagtgccccagctcctgccccagcaggcatgtaccggagcggcgaacggctgctgggcagccacgctctgcctgccgagcagcgggacttcctgcccctggagaccaccaataacaacaacaatcaccaccaacccggggcttgggctcgacgagccggctccagtgcaagcagtccacctagcgccagcagctctccacaccccagcgccgctgttccagcagccgatcccgccgacagtgccagcggcagcagcaacaagcggaagcgggacaacaaggccagcacctacggcctgaactacagcctgctgcaaccttctggaggccgtgcagctggcggaggtagagccgacggtggcggtgtggtgtacagcggcacgccctggaagcggcggaactacaaccagggcgtggtgggcctgcacgaggagatcagcgacttctacgagtacatgagcccacggcccgaagaggagaagatgcggatggaggtggtgaaccggatcgagagcgtgatcaaggagctgtggcctagcgccgacgtgcagatcttcggcagcttcaagaccggcctgtacctgcccaccagcgacatcgacctggtggtgttcggcaagtgggagaacctgcccctgtggaccctggaggaggccctgcggaaacacaaggtggccgacgaggacagcgtgaaggtgctggacaaggccaccgtgcccatcatcaagctgaccgacagcttcaccgaggtgaaggtggacatcagcttcaacgtgcagaacggcgtgagggcagccgacctgatcaaggacttcaccaagaagtaccccgtgctgccctacctggtgctggtgctgaagcagttcctgctgcagcgggacctgaacgaggtgttcaccggcggcatcggcagctacagcctgttcctgatggccgtgagcttcctgcagctgcatccccgtgaggacgcctgcatccccaacaccaactacggcgtgctgctgatcgagttcttcgagctgtacggccggcacttcaactacctgaagaccggcatccggatcaaggacggcggcagctacgtggccaaggacgaggtgcagaagaacatgctggacggctaccggcccagcatgctgtacatcgaggacccactgcagcccggcaacgacgtgggccggagcagctacggcgccatgcaggtgaagcaggccttcgactacgcctacgtggtgctgagccacgccgtgagccccatcgccaagtactaccccaacaacgagaccgagagcatcctgggccggatcatccgggtgaccgacgaggtggccacctaccgggactggatcagcaagcagtggggcctgaagaaccggcccgagcctagctgcaacggccccgtgagctcaagcagcgccacccagagcagcagcagcgacgtggacagcgacgccactccctgcaagacccctaagcagctgctgtgccgtcccagcaccggcaaccgggtgggcagccaggacgtgagcctggagagcagccaggccgtgggcaagatgcagagcacccagaccaccaacaccagcaacagcaccaacaagagccaacacggcagcgcacggctgttccggagcagcagcaagggcttccagggcaccacccagaccagccacggcagcctgatgaccaacaagcagcaccagggcaagagcaacaaccagtactaccacggcaagaagcggaagcacaagcgggacgctcccctgagcgacctgtgccgg

SEQ ID NO: 60 - protein, 1584 aa, Artificial Sequence
  source: /note=“Description of Artificial Sequence Synthetic polypeptide”
NSQPQTRSPFFQRPQIQPPRATIPNSSPSIRPGAQTPTAVYQANQHIMMVNHLPMPYPVPQGPQYCIPQYRHSGPPYVGPPQQYPVQPPGPGPFYPGPGPGDFPNAYGTPFYPSQPVYQSAPIIVPTQQQPPPAKREKKTIRIRDPNQGGKDITEEIMSGGGSRNPTPPIGRPTSTPTPPQQLPSQVPEHSPVVYGTVESAHLAASTPVTAASDQKQEEKPKPDPVLKSPSPVLRLVLSGEKKEQEGQTSETTAIVSIAELPLPPSPTTVSSVARSTIAAPTSSALSSQPIFTTAIDDRCELSSPREDTIPIPSLTSCTETSDPLPTNENDDDICKKPCSVAPNDIPLVSSTNLINEINGVSEKLSATESIVEIVKQEVLPLTLELEILENPPEEMKLECIPAPITPSTVPSFPPTPPTPPASPPHTPVIVPAAATTVSSPSAAITVQRVLEEDESIRTCLSEDAKEIQNKIEVEADGQTEEILDSQNLNSRRSPVPAQIAITVPKTWKKPKDRTRTTEEMLEAELELKAEEELSIDKVLESEQDKMSQGFHPERDPSDLKKVKAVEENGEEAEPVRNGAESVSEGEGIDANSGSTDSSGDGVTFPFKPESWKPTDTEGKKQYDREFLLDFQFMPACIQKPEGLPPISDVVLDKINQPKLPMRTLDPRILPRGPDFTPAFADFGRQTPGGRGVPLLNVGSRRSQPGQRREPRKIITVSVKEDVHLKKAENAWKPSQKRDSQADDPENIKTQELFRKVRSILNKLTPQMFNQLMKQVSGLTVDTEERLKGVIDLVFEKAIDEPSFSVAYANMCRCLVTLKVPMADKPGNTVNFRKLLLNRCQKEFEKDKADDDVFEKKQKELEAASAPEERTRLHDELEEAKDKARRRSIGNIKFIGELFKLKMLTEAIMHDCVVKLLKNHDEESLECLCRLLTTIGKDLDFEKAKPRMDQYFNQMEKIVKERKTSSRIRFMLQDVIDLRLCNWVSRRADQGPKTIEQIHKEAKIEEQEEQRKVQQLMTKEKRRPGVQRVDEGGWNTVQGAKNSRVLDPSKFLKITKPTIDEKIQLVPKAQLGSWGKGSSGGAKASETDALRSSASSLNRFSALQPPAPSGSTPSTPVEFDSRRTLTSRGSMGREKNDKPLPSATARPNTFMRGGSSKDLLDNQSQEEQRREMLETVKQLTGGVDVERNSTEAERNKTRESAKPEISAMSAHDKAALSEEELERKSKSIIDEFLHINDFKEAMQCVEELNAQGLLHVFVRVGVESTLERSQITRDHMGQLLYQLVQSEKLSKQDFFKGFSETLELADDMAIDIPHIWLYLAELVTPMLKEGGISMRELTIEFSKPLLPVGRAGVLLSEILHLLCKQMSHKKVGALWREADLSWKDFLPEGEDVHNFLLEQKLDFIESDSPCSSEALSKKELSAEELYKRLEKLIIEDKANDEQIFDWVEANLDEIQMSSPTFLRALMTAVCKAAIIADSSTFRVDTAVIKQRVPILLKYLDSDTEKELQALYALQASIVKLDQPANLLRMFFDCLYDEEVISEDAFYKWESSKDPAEQNGKGVALKSVTAFFTWLREAEEESEDN

SEQ ID NO: 61 - DNA, 4752 bp, Artificial Sequence
  source: /note=“Description of Artificial Sequence Synthetic polynucleotide”
aacagccaaccccagacacggagccccttcttccagcggccccagatccagccaccacgggcaaccatccccaacagcagccccagcatccgacccggcgcacagacacccaccgccgtgtaccaggccaaccagcacatcatgatggtgaaccacctgcccatgccctaccccgtgccacaaggccctcagtactgcatcccacagtaccggcacagcggtcctccctacgtggggccgccacagcagtaccccgttcagcctcctggaccgggacccttttatccgggacctggacccggagacttccccaacgcctacggcacacccttctaccccagccagccagtgtaccagagcgcgcccatcatcgtgcccacccagcagcagcctccacccgccaagcgggagaagaagaccatccggatccgggaccccaaccagggcggcaaggacatcaccgaggagatcatgagcggaggcggcagtcggaaccccacaccgcccatcggcagacccaccagcaccccaactccaccacagcagctgcccagccaggtgcccgagcacagccccgtggtgtacggcaccgtggaaagcgcccacctggccgctagcacccctgtgaccgccgccagcgaccagaagcaggaggagaagcccaaacccgaccccgtgctgaagtctcccagccccgtgctgcgtctggtgctgagcggcgagaagaaggagcaggagggccagaccagcgagacaaccgccatcgtgagcatcgccgagctgccactgcctcccagtcccaccaccgtgagcagcgtggcccggagcaccatcgcagcgcccacaagcagcgccctgagcagccagcccatcttcaccaccgccatcgacgaccggtgcgagctgagcagtccccgggaggacaccatccccatccccagcctgaccagctgcaccgagaccagcgacccactgcccaccaacgagaacgacgacgacatctgcaagaagccctgcagcgtggcacccaacgacatcccgctggtgagcagcaccaacctgatcaacgagatcaacggcgttagcgagaaactgtctgccaccgagagcatcgtggagatcgtcaagcaggaggtgctgcccctgaccctggagctagagatcctggagaaccctccagaggagatgaagctggagtgcatccccgccccaatcaccccgagcaccgtgcccagctttcctccaaccccacccaccccaccagccagtccacctcacactcccgtgatcgtgcctgccgccgctaccaccgtgtctagccccagcgccgccatcaccgtccagcgggtactggaggaggacgagagcatccggacctgcctgagcgaagacgccaaggagatccagaacaagatcgaggtggaggccgacggccagaccgaggaaatcctggacagccagaacctgaattcccggcgaagccctgtgcccgcacagatcgccatcacagtgcccaagacctggaagaagcccaaggaccggacccggaccacagaggaaatgctggaggccgagctggagctgaaggccgaggaagagctgagcatcgacaaggtgctggagagcgagcaggacaagatgagccagggcttccaccctgagcgggaccccagcgacctgaagaaggtgaaggccgtggaggagaacggcgaggaggcagagcctgtgcggaacggggccgagagcgtgagcgagggcgagggcatcgacgccaacagcggcagcactgacagcagcggcgacggcgtgaccttccccttcaagcccgaaagctggaagcccaccgacaccgaaggaaagaagcagtacgaccgggagttcctgctggacttccagttcatgcccgcctgcatccagaaacccgaggggctgccaccaatcagcgacgtggtgctggacaagatcaaccagcccaagctgcccatgcggaccctggatcctcggatccttcccagaggccccgacttcactcccgccttcgccgacttcggcaggcagaccccaggtggtcggggtgtgcccctgctgaacgtgggcagccgtcggagccagcctggccaacggcgggaaccccggaagatcatcaccgtgagcgtgaaggaggacgtgcatctgaagaaggccgagaacgcctggaaacccagccagaagcgggacagccaagccgacgaccccgagaacatcaagacccaggagctgttccggaaggttcggagcatcctgaacaagctgacaccccagatgttcaaccagctgatgaagcaggtgagcggcctgaccgtggacaccgaggagcggctgaagggcgtgatcgacctggtgtttgagaaagccattgacgagcccagcttcagcgtggcctacgccaacatgtgccggtgcctggtgaccctgaaggtgcccatggccgacaagcccggcaacaccgtgaacttccggaagctgctgctgaaccggtgccagaaggagttcgagaaggacaaggcagacgacgacgtgttcgagaagaagcagaaagagttagaggccgcaagcgcccctgaggagcggacccggctgcacgacgagctggaggaggccaaagacaaggcccggcggaggagcatcggcaacatcaagttcatcggcgagctgttcaagctgaagatgctgaccgaggccatcatgcacgactgcgtggtgaagctgctgaagaaccacgacgaggagagcctggagtgcctgtgccggctgctgaccaccatcggcaaggacctggacttcgagaaggccaagccccggatggaccagtacttcaaccagatggagaagatcgtgaaggagcggaagaccagcagccggatccggttcatgctgcaggacgttatcgacctgcggctgtgcaactgggtgagccggagggctgaccagggccccaagaccatcgagcagatccacaaggaggctaagatcgaggagcaggaagagcagcggaaggtgcagcagctgatgaccaaagagaagcggaggcctggcgtgcagcgagtggacgagggcggctggaacaccgtgcagggcgccaagaacagccgggtgctggaccccagcaagttcctgaagatcaccaagcccaccatcgacgagaagatccagctggtgcccaaggcccagctcggcagctggggcaagggcagctcaggtggcgccaaggccagcgagaccgacgccctgagaagcagcgccagcagcctgaaccggttctctgccctgcagcctcctgcccctagcggctccaccccaagcacacccgtggagttcgacagccggcggaccctgaccagccggggctccatgggtcgcgagaagaacgacaagcccttacccagcgcaaccgctcggcccaacaccttcatgcggggcggcagctccaaggacctgctggacaaccagagccaggaggagcagcggcgggagatgctggagaccgtgaagcagctgaccggaggcgtggacgtggagcggaacagcaccgaggccgagcggaacaagacccgggagagcgccaagcccgagatcagcgccatgagcgcccacgacaaggccgccctgagcgaggaggagctggaacggaagagcaagagcatcatcgacgagttcctgcacatcaacgacttcaaggaggccatgcagtgcgtggaggagctgaacgcccagggcctgctgcacgtgttcgtgcgggtgggcgtggagagcaccctggagcggagccagatcacccgggaccacatgggccagctgctgtaccaactggtgcagagcgagaagctgagcaagcaggacttcttcaagggcttcagcgagactctggagctggccgacgacatggccatcgacattccccacatctggctgtacctggccgagcttgtgaccccaatgctgaaggagggcggcatcagcatgcgggagctgaccatcgagttcagcaagcccctcttgccagtcggcagagcaggagtgctgctgagcgagatcctgcacctgctgtgcaagcagatgagccacaagaaggtgggcgccctgtggagggaggccgacctgagctggaaggacttcctgcccgagggcgaagacgtgcacaacttcttgctggagcagaagctggacttcatcgagagcgacagcccctgcagcagcgaggccctgagcaagaaggaactgagcgccgaggagctgtacaagcggctggagaagctgatcatcgaggacaaggccaacgacgagcagatcttcgactgggtggaggccaacctggacgagatccagatgagcagccccaccttcctgcgggccctgatgaccgccgtgtgcaaggccgccatcatcgccgacagcagcaccttccgggtggacaccgccgtgatcaagcagcgggtgcccatcctgctgaagtacctggacagcgacaccgagaaggagctgcaggccctgtacgccctgcaggccagcatcgtgaagctggaccagcccgccaacctgctgcggatgttcttcgactgcctgtacgacgaagaggtgatcagcgaggacgccttctacaagtgggagagcagcaaggaccccgccgagcagaacggcaagggcgtggccctgaagagcgtgaccgccttcttcacctggctgcgagaggccgaggaggagagcgaggacaac

SEQ ID NO: 62 - protein, 540 aa, Artificial Sequence
  source: /note=“Description of Artificial Sequence Synthetic polypeptide”
GEQKYEYKSDQWKPLNLEEKKRYDREFLLGFQFIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQA

SEQ ID NO: 63 - DNA, 1620 bp, Artificial Sequence
  source: /note=“Description of Artificial Sequence Synthetic polynucleotide”
ggcgagcagaagtacgagtacaagagcgaccagtggaagcctctgaacctggaggagaagaagagatacgacagagagttcctgctgggcttccagttcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagct

SEQ ID NO: 64 - protein, 1584 aa, Artificial Sequence; identical to SEQ ID NO: 60
  source: /note=“Description of Artificial Sequence Synthetic polypeptide”

SEQ ID NO: 65 - DNA, 4755 bp, Artificial Sequence
  source: /note=“Description of Artificial Sequence Synthetic polynucleotide”
agcaacagccaaccccagacacggagccccttcttccagcggccccagatccagccaccacgggcaaccatccccaacagcagccccagcatccgacccggcgcacagacacccaccgccgtgtaccaggccaaccagcacatcatgatggtgaaccacctgcccatgccctaccccgtgccacaaggccctcagtactgcatcccacagtaccggcacagcggtcctccctacgtggggccgccacagcagtaccccgttcagcctcctggaccgggacccttttatccgggacctggacccggagacttccccaacgcctacggcacacccttctaccccagccagccagtgtaccagagcgcgcccatcatcgtgcccacccagcagcagcctccacccgccaagcgggagaagaagaccatccggatccgggaccccaaccagggcggcaaggacatcaccgaggagatcatgagcggaggcggcagtcggaaccccacaccgcccatcggcagacccaccagcaccccaactccaccacagcagctgcccagccaggtgcccgagcacagccccgtggtgtacggcaccgtggaaagcgcccacctggccgctagcacccctgtgaccgccgccagcgaccagaagcaggaggagaagcccaaacccgaccccgtgctgaagtctcccagccccgtgctgcgtctggtgctgagcggcgagaagaaggagcaggagggccagaccagcgagacaaccgccatcgtgagcatcgccgagctgccactgcctcccagtcccaccaccgtgagcagcgtggcccggagcaccatcgcagcgcccacaagcagcgccctgagcagccagcccatcttcaccaccgccatcgacgaccggtgcgagctgagcagtccccgggaggacaccatccccatccccagcctgaccagctgcaccgagaccagcgacccactgcccaccaacgagaacgacgacgacatctgcaagaagccctgcagcgtggcacccaacgacatcccgctggtgagcagcaccaacctgatcaacgagatcaacggcgttagcgagaaactgtctgccaccgagagcatcgtggagatcgtcaagcaggaggtgctgcccctgaccctggagctagagatcctggagaaccctccagaggagatgaagctggagtgcatccccgccccaatcaccccgagcaccgtgcccagctttcctccaaccccacccaccccaccagccagtccacctcacactcccgtgatcgtgcctgccgccgctaccaccgtgtctagccccagcgccgccatcaccgtccagcgggtactggaggaggacgagagcatccggacctgcctgagcgaagacgccaaggagatccagaacaagatcgaggtggaggccgacggccagaccgaggaaatcctggacagccagaacctgaattcccggcgaagccctgtgcccgcacagatcgccatcacagtgcccaagacctggaagaagcccaaggaccggacccggaccacagaggaaatgctggaggccgagctggagctgaaggccgaggaagagctgagcatcgacaaggtgctggagagcgagcaggacaagatgagccagggcttccaccctgagcgggaccccagcgacctgaagaaggtgaaggccgtggaggagaacggcgaggaggcagagcctgtgcggaacggggccgagagcgtgagcgagggcgagggcatcgacgccaacagcggcagcactgacagcagcggcgacggcgtgaccttccccttcaagcccgaaagctggaagcccaccgacaccgaaggaaagaagcagtacgaccgggagttcctgctggacttccagttcatgcccgcctgcatccagaaacccgaggggctgccaccaatcagcgacgtggtgctggacaagatcaaccagcccaagctgcccatgcggaccctggatcctcggatccttcccagaggccccgacttcactcccgccttcgccgacttcggcaggcagaccccaggtggtcggggtgtgcccctgctgaacgtgggcagccgtcggagccagcctggccaacggcgggaaccccggaagatcatcaccgtgagcgtgaaggaggacgtgcatctgaagaaggccgagaacgcctggaaacccagccagaagcgggacagccaagccgacgaccccgagaacatcaagacccaggagctgttccggaaggttcggagcatcctgaacaagctgacaccccagatgttcaaccagctgatgaagcaggtgagcggcctgaccgtggacaccgaggagcggctgaagggcgtgatcgacctggtgtttgagaaagccattgacgagcccagcttcagcgtggcctacgccaacatgtgccggtgcctggtgaccctgaaggtgcccatggccgacaagcccggcaacaccgtgaacttccggaagctgctgctgaaccggtgccagaaggagttcgagaaggacaaggcagacgacgacgtgttcgagaagaagcagaaagagttagaggccgcaagcgcccctgaggagcggacccggctgcacgacgagctggaggaggccaaagacaaggcccggcggaggagcatcggcaacatcaagttcatcggcgagctgttcaagctgaagatgctgaccgaggccatcatgcacgactgcgtggtgaagctgctgaagaaccacgacgaggagagcctggagtgcctgtgccggctgctgaccaccatcggcaaggacctggacttcgagaaggccaagccccggatggaccagtacttcaaccagatggagaagatcgtgaaggagcggaagaccagcagccggatccggttcatgctgcaggacgttatcgacctgcggctgtgcaactgggtgagccggagggctgaccagggccccaagaccatcgagcagatccacaaggaggctaagatcgaggagcaggaagagcagcggaaggtgcagcagctgatgaccaaagagaagcggaggcctggcgtgcagcgagtggacgagggcggctggaacaccgtgcagggcgccaagaacagccgggtgctggaccccagcaagttcctgaagatcaccaagcccaccatcgacgagaagatccagctggtgcccaaggcccagctcggcagctggggcaagggcagctcaggtggcgccaaggccagcgagaccgacgccctgagaagcagcgccagcagcctgaaccggttctctgccctgcagcctcctgcccctagcggctccaccccaagcacacccgtggagttcgacagccggcggaccctgaccagccggggctccatgggtcgcgagaagaacgacaagcccttacccagcgcaaccgctcggcccaacaccttcatgcggggcggcagctccaaggacctgctggacaaccagagccaggaggagcagcggcgggagatgctggagaccgtgaagcagctgaccggaggcgtggacgtggagcggaacagcaccgaggccgagcggaacaagacccgggagagcgccaagcccgagatcagcgccatgagcgcccacgacaaggccgccctgagcgaggaggagctggaacggaagagcaagagcatcatcgacgagttcctgcacatcaacgacttcaaggaggccatgcagtgcgtggaggagctgaacgcccagggcctgctgcacgtgttcgtgcgggtgggcgtggagagcaccctggagcggagccagatcacccgggaccacatgggccagctgctgtaccaactggtgcagagcgagaagctgagcaagcaggacttcttcaagggcttcagcgagactctggagctggccgacgacatggccatcgacattccccacatctggctgtacctggccgagcttgtgaccccaatgctgaaggagggcggcatcagcatgcgggagctgaccatcgagttcagcaagcccctcttgccagtcggcagagcaggagtgctgctgagcgagatcctgcacctgctgtgcaagcagatgagccacaagaaggtgggcgccctgtggagggaggccgacctgagctggaaggacttcctgcccgagggcgaagacgtgcacaacttcttgctggagcagaagctggacttcatcgagagcgacagcccctgcagcagcgaggccctgagcaagaaggaactgagcgccgaggagctgtacaagcggctggagaagctgatcatcgaggacaaggccaacgacgagcagatcttcgactgggtggaggccaacctggacgagatccagatgagcagccccaccttcctgcgggccctgatgaccgccgtgtgcaaggccgccatcatcgccgacagcagcaccttccgggtggacaccgccgtgatcaagcagcgggtgcccatcctgctgaagtacctggacagcgacaccgagaaggagctgcaggccctgtacgccctgcaggccagcatcgtgaagctggaccagcccgccaacctgctgcggatgttcttcgactgcctgtacgacgaagaggtgatcagcgaggacgccttctacaagtgggagagcagcaaggaccccgccgagcagaacggcaagggcgtggccctgaagagcgtgaccgccttcttcacctggctgcgagaggccgaggaggagagcgaggacaac

SEQ ID NO: 66 - protein, 418 aa, Artificial Sequence
  source: /note=“Description of Artificial Sequence Synthetic polypeptide”
RTLDPRILPRGPDFTPAFADFGRQTPGGRGVPLLNVGSRRSQPGQRREPRKIITVSVKEDVHLKKAENAWKPSQKRDSQADDPENIKTQELFRKVRSILNKLTPQMFNQLMKQVSGLTVDTEERLKGVIDLVFEKAIDEPSFSVAYANMCRCLVTLKVPMADKPGNTVNFRKLLLNRCQKEFEKDKADDDVFEKKQKELEAASAPEERTRLHDELEEAKDKARRRSIGNIKFIGELFKLKMLTEAIMHDCVVKLLKNHDEESLECLCRLLTTIGKDLDFEKAKPRMDQYFNQMEKIVKERKTSSRIRFMLQDVIDLRLCNWVSRRADQGPKTIEQIHKEAKIEEQEEQRKVQQLMTKEKRRPGVQRVDEGGWNTVQGAKNSRVLDPSKFLKITKPTIDEKIQLVPKAQLGSWGKGSSG

SEQ ID NO: 67 - DNA, 1257 bp, Artificial Sequence
  source: /note=“Description of Artificial Sequence Synthetic polynucleotide”
agccggaccctggatcctcggatccttcccagaggccccgacttcactcccgccttcgccgacttcggcaggcagaccccaggtggtcggggtgtgcccctgctgaacgtgggcagccgtcggagccagcctggccaacggcgggaaccccggaagatcatcaccgtgagcgtgaaggaggacgtgcatctgaagaaggccgagaacgcctggaaacccagccagaagcgggacagccaagccgacgaccccgagaacatcaagacccaggagctgttccggaaggttcggagcatcctgaacaagctgacaccccagatgttcaaccagctgatgaagcaggtgagcggcctgaccgtggacaccgaggagcggctgaagggcgtgatcgacctggtgtttgagaaagccattgacgagcccagcttcagcgtggcctacgccaacatgtgccggtgcctggtgaccctgaaggtgcccatggccgacaagcccggcaacaccgtgaacttccggaagctgctgctgaaccggtgccagaaggagttcgagaaggacaaggcagacgacgacgtgttcgagaagaagcagaaagagttagaggccgcaagcgcccctgaggagcggacccggctgcacgacgagctggaggaggccaaagacaaggcccggcggaggagcatcggcaacatcaagttcatcggcgagctgttcaagctgaagatgctgaccgaggccatcatgcacgactgcgtggtgaagctgctgaagaaccacgacgaggagagcctggagtgcctgtgccggctgctgaccaccatcggcaaggacctggacttcgagaaggccaagccccggatggaccagtacttcaaccagatggagaagatcgtgaaggagcggaagaccagcagccggatccggttcatgctgcaggacgttatcgacctgcggctgtgcaactgggtgagccggagggctgaccagggccccaagaccatcgagcagatccacaaggaggctaagatcgaggagcaggaagagcagcggaaggtgcagcagctgatgaccaaagagaagcggaggcctggcgtgcagcgagtggacgagggcggctggaacaccgtgcagggcgccaagaacagccgggtgctggaccccagcaagttcctgaagatcaccaagcccaccatcgacgagaagatccagctggtgcccaaggcccagctcggcagctggggcaagggcagctcaggt

SEQ ID NO: 68 - protein, 600 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKAIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 69 - DNA, 1800 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaaggcgataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 70 - protein, 662 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSGEQKYEYKSDQWKPLNLEEKKRYDREFLLGFQFIFASMQKPEGLPHISDVVLDKANKTPLRPLDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQA

SEQ ID NO: 71 - DNA, 1986 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcggcgagcagaagtacgagtacaagagcgaccagtggaagcctctgaacctggaggagaagaagagatacgacagagagttcctgctgggcttccagttcatattcgcgagcatgcagaagcctgaaggtctgcctcacatcagcgacgtggtgctggacaaggccaacaagacccctcttagacctctggaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagct

SEQ ID NO: 72 - protein, 1706 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSNSQPQTRSPFFQRPQIQPPRATIPNSSPSIRPGAQTPTAVYQANQHIMMVNHLPMPYPVPQGPQYCIPQYRHSGPPYVGPPQQYPVQPPGPGPFYPGPGPGDFPNAYGTPFYPSQPVYQSAPIIVPTQQQPPPAKREKKTIRIRDPNQGGKDITEEIMSGGGSRNPTPPIGRPTSTPTPPQQLPSQVPEHSPVVYGTVESAHLAASTPVTAASDQKQEEKPKPDPVLKSPSPVLRLVLSGEKKEQEGQTSETTAIVSIAELPLPPSPTTVSSVARSTIAAPTSSALSSQPIFTTAIDDRCELSSPREDTIPIPSLTSCTETSDPLPTNENDDDICKKPCSVAPNDIPLVSSTNLINEINGVSEKLSATESIVEIVKQEVLPLTLELEILENPPEEMKLECIPAPITPSTVPSFPPTPPTPPASPPHTPVIVPAAATTVSSPSAAITVQRVLEEDESIRTCLSEDAKEIQNKIEVEADGQTEEILDSQNLNSRRSPVPAQIAITVPKTWKKPKDRTRTTEEMLEAELELKAEEELSIDKVLESEQDKMSQGFHPERDPSDLKKVKAVEENGEEAEPVRNGAESVSEGEGIDANSGSTDSSGDGVTFPFKPESWKPTDTEGKKQYDREFLLDFQFMPACIQKPEGLPPISDVVLDKINQPKLPMRTLDPRILPRGPDFTPAFADFGRQTPGGRGVPLLNVGSRRSQPGQRREPRKIITVSVKEDVHLKKAENAWKPSQKRDSQADDPENIKTQELFRKVRSILNKLTPQMFNQLMKQVSGLTVDTEERLKGVIDLVFEKAIDEPSFSVAYANMCRCLVTLKVPMADKPGNTVNFRKLLLNRCQKEFEKDKADDDVFEKKQKELEAASAPEERTRLHDELEEAKDKARRRSIGNIKFIGELFKLKMLTEAIMHDCVVKLLKNHDEESLECLCRLLTTIGKDLDFEKAKPRMDQYFNQMEKIVKERKTSSRIRFMLQDVIDLRLCNWVSRRADQGPKTIEQIHKEAKIEEQEEQRKVQQLMTKEKRRPGVQRVDEGGWNTVQGAKNSRVLDPSKFLKITKPTIDEKIQLVPKAQLGSWGKGSSGGAKASETDALRSSASSLNRFSALQPPAPSGSTPSTPVEFDSRRTLTSRGSMGREKNDKPLPSATARPNTFMRGGSSKDLLDNQSQEEQRREMLETVKQLTGGVDVERNSTEAERNKTRESAKPEISAMSAHDKAALSEEELERKSKSIIDEFLHINDFKEAMQCVEELNAQGLLHVFVRVGVESTLERSQITRDHMGQLLYQLVQSEKLSKQDFFKGFSETLELADDMAIDIPHIWLYLAELVTPMLKEGGISMRELTIEFSKPLLPVGRAGVLLSEILHLLCKQMSHKKVGALWREADLSWKDFLPEGEDVHNFLLEQKLDFIESDSPCSSEALSKKELSAEELYKRLEKLIIEDKANDEQIFDWVEANLDEIQMSSPTFLRALMTAVCKAAIIADSSTFRVDTAVIKQRVPILLKYLDSDTEKELQALYALQASIVKLDQPANLLRMFFDCLYDEEVISEDAFYKWESSKDPAEQNGKGVALKSVTAFFTWLREAEEESEDN

SEQ ID NO: 73 - DNA, 5118 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcaacagccaaccccagacacggagccccttcttccagcggccccagatccagccaccacgggcaaccatccccaacagcagccccagcatccgacccggcgcacagacacccaccgccgtgtaccaggccaaccagcacatcatgatggtgaaccacctgcccatgccctaccccgtgccacaaggccctcagtactgcatcccacagtaccggcacagcggtcctccctacgtggggccgccacagcagtaccccgttcagcctcctggaccgggacccttttatccgggacctggacccggagacttccccaacgcctacggcacacccttctaccccagccagccagtgtaccagagcgcgcccatcatcgtgcccacccagcagcagcctccacccgccaagcgggagaagaagaccatccggatccgggaccccaaccagggcggcaaggacatcaccgaggagatcatgagcggaggcggcagtcggaaccccacaccgcccatcggcagacccaccagcaccccaactccaccacagcagctgcccagccaggtgcccgagcacagccccgtggtgtacggcaccgtggaaagcgcccacctggccgctagcacccctgtgaccgccgccagcgaccagaagcaggaggagaagcccaaacccgaccccgtgctgaagtctcccagccccgtgctgcgtctggtgctgagcggcgagaagaaggagcaggagggccagaccagcgagacaaccgccatcgtgagcatcgccgagctgccactgcctcccagtcccaccaccgtgagcagcgtggcccggagcaccatcgcagcgcccacaagcagcgccctgagcagccagcccatcttcaccaccgccatcgacgaccggtgcgagctgagcagtccccgggaggacaccatccccatccccagcctgaccagctgcaccgagaccagcgacccactgcccaccaacgagaacgacgacgacatctgcaagaagccctgcagcgtggcacccaacgacatcccgctggtgagcagcaccaacctgatcaacgagatcaacggcgttagcgagaaactgtctgccaccgagagcatcgtggagatcgtcaagcaggaggtgctgcccctgaccctggagctagagatcctggagaaccctccagaggagatgaagctggagtgcatccccgccccaatcaccccgagcaccgtgcccagctttcctccaaccccacccaccccaccagccagtccacctcacactcccgtgatcgtgcctgccgccgctaccaccgtgtctagccccagcgccgccatcaccgtccagcgggtactggaggaggacgagagcatccggacctgcctgagcgaagacgccaaggagatccagaacaagatcgaggtggaggccgacggccagaccgaggaaatcctggacagccagaacctgaattcccggcgaagccctgtgcccgcacagatcgccatcacagtgcccaagacctggaagaagcccaaggaccggacccggaccacagaggaaatgctggaggccgagctggagctgaaggccgaggaagagctgagcatcgacaaggtgctggagagcgagcaggacaagatgagccagggcttccaccctgagcgggaccccagcgacctgaagaaggtgaaggccgtggaggagaacggcgaggaggcagagcctgtgcggaacggggccgagagcgtgagcgagggcgagggcatcgacgccaacagcggcagcactgacagcagcggcgacggcgtgaccttccccttcaagcccgaaagctggaagcccaccgacaccgaaggaaagaagcagtacgaccgggagttcctgctggacttccagttcatgcccgcctgcatccagaaacccgaggggctgccaccaatcagcgacgtggtgctggacaagatcaaccagcccaagctgcccatgcggaccctggatcctcggatccttcccagaggccccgacttcactcccgccttcgccgacttcggcaggcagaccccaggtggtcggggtgtgcccctgctgaacgtgggcagccgtcggagccagcctggccaacggcgggaaccccggaagatcatcaccgtgagcgtgaaggaggacgtgcatctgaagaaggccgagaacgcctggaaacccagccagaagcgggacagccaagccgacgaccccgagaacatcaagacccaggagctgttccggaaggttcggagcatcctgaacaagctgacaccccagatgttcaaccagctgatgaagcaggtgagcggcctgaccgtggacaccgaggagcggctgaagggcgtgatcgacctggtgtttgagaaagccattgacgagcccagcttcagcgtggcctacgccaacatgtgccggtgcctggtgaccctgaaggtgcccatggccgacaagcccggcaacaccgtgaacttccggaagctgctgctgaaccggtgccagaaggagttcgagaaggacaaggcagacgacgacgtgttcgagaagaagcagaaagagttagaggccgcaagcgcccctgaggagcggacccggctgcacgacgagctggaggaggccaaagacaaggcccggcggaggagcatcggcaacatcaagttcatcggcgagctgttcaagctgaagatgctgaccgaggccatcatgcacgactgcgtggtgaagctgctgaagaaccacgacgaggagagcctggagtgcctgtgccggctgctgaccaccatcggcaaggacctggacttcgagaaggccaagccccggatggaccagtacttcaaccagatggagaagatcgtgaaggagcggaagaccagcagccggatccggttcatgctgcaggacgttatcgacctgcggctgtgcaactgggtgagccggagggctgaccagggccccaagaccatcgagcagatccacaaggaggctaagatcgaggagcaggaagagcagcggaaggtgcagcagctgatgaccaaagagaagcggaggcctggcgtgcagcgagtggacgagggcggctggaacaccgtgcagggcgccaagaacagccgggtgctggaccccagcaagttcctgaagatcaccaagcccaccatcgacgagaagatccagctggtgcccaaggcccagctcggcagctggggcaagggcagctcaggtggcgccaaggccagcgagaccgacgccctgagaagcagcgccagcagcctgaaccggttctctgccctgcagcctcctgcccctagcggctccaccccaagcacacccgtggagttcgacagccggcggaccctgaccagccggggctccatgggtcgcgagaagaacgacaagcccttacccagcgcaaccgctcggcccaacaccttcatgcggggcggcagctccaaggacctgctggacaaccagagccaggaggagcagcggcgggagatgctggagaccgtgaagcagctgaccggaggcgtggacgtggagcggaacagcaccgaggccgagcggaacaagacccgggagagcgccaagcccgagatcagcgccatgagcgcccacgacaaggccgccctgagcgaggaggagctggaacggaagagcaagagcatcatcgacgagttcctgcacatcaacgacttcaaggaggccatgcagtgcgtggaggagctgaacgcccagggcctgctgcacgtgttcgtgcgggtgggcgtggagagcaccctggagcggagccagatcacccgggaccacatgggccagctgctgtaccaactggtgcagagcgagaagctgagcaagcaggacttcttcaagggcttcagcgagactctggagctggccgacgacatggccatcgacattccccacatctggctgtacctggccgagcttgtgaccccaatgctgaaggagggcggcatcagcatgcgggagctgaccatcgagttcagcaagcccctcttgccagtcggcagagcaggagtgctgctgagcgagatcctgcacctgctgtgcaagcagatgagccacaagaaggtgggcgccctgtggagggaggccgacctgagctggaaggacttcctgcccgagggcgaagacgtgcacaacttcttgctggagcagaagctggacttcatcgagagcgacagcccctgcagcagcgaggccctgagcaagaaggaactgagcgccgaggagctgtacaagcggctggagaagctgatcatcgaggacaaggccaacgacgagcagatcttcgactgggtggaggccaacctggacgagatccagatgagcagccccaccttcctgcgggccctgatgaccgccgtgtgcaaggccgccatcatcgccgacagcagcaccttccgggtggacaccgccgtgatcaagcagcgggtgcccatcctgctgaagtacctggacagcgacaccgagaaggagctgcaggccctgtacgccctgcaggccagcatcgtgaagctggaccagcccgccaacctgctgcggatgttcttcgactgcctgtacgacgaagaggtgatcagcgaggacgccttctacaagtgggagagcagcaaggaccccgccgagcagaacggcaagggcgtggccctgaagagcgtgaccgccttcttcacctggctgcgagaggccgaggaggagagcgaggacaac

SEQ ID NO: 74 - protein, 540 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSRTLDPRILPRGPDFTPAFADFGRQTPGGRGVPLLNVGSRRSQPGQRREPRKIITVSVKEDVHLKKAENAWKPSQKRDSQADDPENIKTQELFRKVRSILNKLTPQMFNQLMKQVSGLTVDTEERLKGVIDLVFEKAIDEPSFSVAYANMCRCLVTLKVPMADKPGNTVNFRKLLLNRCQKEFEKDKADDDVFEKKQKELEAASAPEERTRLHDELEEAKDKARRRSIGNIKFIGELFKLKMLTEAIMHDCVVKLLKNHDEESLECLCRLLTTIGKDLDFEKAKPRMDQYFNQMEKIVKERKTSSRIRFMLQDVIDLRLCNWVSRRADQGPKTIEQIHKEAKIEEQEEQRKVQQLMTKEKRRPGVQRVDEGGWNTVQGAKNSRVLDPSKFLKITKPTIDEKIQLVPKAQLGSWGKGSSG

SEQ ID NO: 75 - DNA, 1620 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagccggaccctggatcctcggatccttcccagaggccccgacttcactcccgccttcgccgacttcggcaggcagaccccaggtggtcggggtgtgcccctgctgaacgtgggcagccgtcggagccagcctggccaacggcgggaaccccggaagatcatcaccgtgagcgtgaaggaggacgtgcatctgaagaaggccgagaacgcctggaaacccagccagaagcgggacagccaagccgacgaccccgagaacatcaagacccaggagctgttccggaaggttcggagcatcctgaacaagctgacaccccagatgttcaaccagctgatgaagcaggtgagcggcctgaccgtggacaccgaggagcggctgaagggcgtgatcgacctggtgtttgagaaagccattgacgagcccagcttcagcgtggcctacgccaacatgtgccggtgcctggtgaccctgaaggtgcccatggccgacaagcccggcaacaccgtgaacttccggaagctgctgctgaaccggtgccagaaggagttcgagaaggacaaggcagacgacgacgtgttcgagaagaagcagaaagagttagaggccgcaagcgcccctgaggagcggacccggctgcacgacgagctggaggaggccaaagacaaggcccggcggaggagcatcggcaacatcaagttcatcggcgagctgttcaagctgaagatgctgaccgaggccatcatgcacgactgcgtggtgaagctgctgaagaaccacgacgaggagagcctggagtgcctgtgccggctgctgaccaccatcggcaaggacctggacttcgagaaggccaagccccggatggaccagtacttcaaccagatggagaagatcgtgaaggagcggaagaccagcagccggatccggttcatgctgcaggacgttatcgacctgcggctgtgcaactgggtgagccggagggctgaccagggccccaagaccatcgagcagatccacaaggaggctaagatcgaggagcaggaagagcagcggaaggtgcagcagctgatgaccaaagagaagcggaggcctggcgtgcagcgagtggacgagggcggctggaacaccgtgcagggcgccaagaacagccgggtgctggaccccagcaagttcctgaagatcaccaagcccaccatcgacgagaagatccagctggtgcccaaggcccagctcggcagctggggcaagggcagctcaggt

SEQ ID NO: 76 - protein, 483 aa, Artificial Sequence
  source: /note=“Description of Artificial Sequence Synthetic polypeptide”
FPNSILGRPPFTPNHQQHNNFFTLSPTVYSHQQLIDAQFNFQNADLSRAVSLQQLTYGNVSPIQTSASPLFRGRKRLSDEKNLPLDGKRQRFHSPHQEPTVVNQIVPLSGERRYSMPPLFHTHYVPDIVRCVPPFREIAFLEPREITLPEAKDKLSQQILELFETCQQQISDLKKKELCRTQLQREIQLLFPQSRLFLVGSSLNGFGTRSSDGDLCLVVKEEPCFFQVNQKTEARHILTLVHKHFCTRLSGYIERPQLIRAKVPIVKFRDKVSCVEFDLNVNNIVGIRNTFLLRTYAYLENRVRPLVLVIKKWASHHQINDASRGTLSSYSLVLMVLHYLQTLPEPILPSLQKIYPESFSPAIQLHLVHQAPCNVPPYLSKNESNLGDLLLGFLKYYATEFDWNSQMISVREAKAIPRPDGIEWRNKYICVEEPFDGTNTARAVHEKQKFDMIKDQFLKSWHRLKNKRDLNSILPVRAAVLKR

SEQ ID NO: 77 - DNA, 1449 bp, Artificial Sequence
  source: /note=“Description of Artificial Sequence Synthetic polynucleotide”
ttccctaacagcatcctgggcagacctcctttcacccctaaccaccagcagcacaacaacttcttcaccctgagccctaccgtgtacagccatcagcagctgatcgacgcccagttcaacttccagaacgccgacctgagcagagccgtgagcctgcagcaactcacctacggcaacgttagcccaatccagaccagcgccagccctctgttcagaggcagaaagagactgagcgacgagaagaacctgcctctggacggcaagagacagagattccacagccctcaccaggagccaacagtggtgaaccagatcgtgcctctgagcggcgagagaagatacagcatgcctccactgttccacacccactacgtgcctgacatcgtgaggtgcgtgccacctttcagagagatcgccttcctggagcctcgcgagattaccctgcctgaggccaaggacaagctgagccagcagattctggaactgttcgagacttgccaacaacaaattagcgacctgaagaagaaggagctgtgcagaacccagctgcagcgcgagatccagctgctgttccctcagagcagactgttcctggtgggcagcagcctgaacggcttcggcaccagaagcagcgacggcgacctgtgcctggtggtgaaggaggagccttgcttcttccaagtaaatcagaagaccgaggccagacacatcctgaccctggtgcacaagcacttctgcaccagactgtccggctacatcgagcgccctcagcttataagagccaaggtgcctatcgtgaagttcagagacaaggtgagctgcgtggagttcgatctgaacgtgaacaacatcgtgggcatcagaaacaccttcctgctgagaacctacgcctacctggagaacagagtcagacctctggtgctggtgatcaagaagtgggcttctcatcaccagatcaacgacgcctcccgcggtactctgtctagctacagcctcgtactgatggtgctgcactacctgcagactctccctgaacctatcctgcctagtctgcagaagatctaccctgagagcttcagccctgccatccaattgcatctcgttcaccaggccccttgcaacgttcctccttatttgagcaagaacgagagcaacctgggagatctcctgctgggcttcctgaagtactacgccaccgaattcgattggaacagccagatgatcagcgtgagagaagcaaaggccatccctagacctgacggcatcgagtggagaaacaagtacatctgtgtggaggaacctttcgacggcaccaacaccgcccgcgcagtacacgagaagcagaagttcgacatgatcaaggaccagttcctcaagagctggcacagactgaagaacaagagagacttgaatagcatcttgccggtgagagccgccgtgctgaagaga

SEQ ID NO: 78 - protein, 605 aa, Artificial Sequence
  source: /note=“Description of Artificial Sequence Synthetic polypeptide”
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSFPNSILGRPPFTPNHQQHNNFFTLSPTVYSHQQLIDAQFNFQNADLSRAVSLQQLTYGNVSPIQTSASPLFRGRKRLSDEKNLPLDGKRQRFHSPHQEPTVVNQIVPLSGERRYSMPPLFHTHYVPDIVRCVPPFREIAFLEPREITLPEAKDKLSQQILELFETCQQQISDLKKKELCRTQLQREIQLLFPQSRLFLVGSSLNGFGTRSSDGDLCLVVKEEPCFFQVNQKTEARHILTLVHKHFCTRLSGYIERPQLIRAKVPIVKFRDKVSCVEFDLNVNNIVGIRNTFLLRTYAYLENRVRPLVLVIKKWASHHQINDASRGTLSSYSLVLMVLHYLQTLPEPILPSLQKIYPESFSPAIQLHLVHQAPCNVPPYLSKNESNLGDLLLGFLKYYATEFDWNSQMISVREAKAIPRPDGIEWRNKYICVEEPFDGTNTARAVHEKQKFDMIKDQFLKSWHRLKNKRDLNSILPVRAAVLKR

SEQ ID NO: 79 - DNA, 1815 bp, Artificial Sequence
  source: /note=“Description of Artificial Sequence Synthetic polynucleotide”
atggccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagcttccctaacagcatcctgggcagacctcctttcacccctaaccaccagcagcacaacaacttcttcaccctgagccctaccgtgtacagccatcagcagctgatcgacgcccagttcaacttccagaacgccgacctgagcagagccgtgagcctgcagcaactcacctacggcaacgttagcccaatccagaccagcgccagccctctgttcagaggcagaaagagactgagcgacgagaagaacctgcctctggacggcaagagacagagattccacagccctcaccaggagccaacagtggtgaaccagatcgtgcctctgagcggcgagagaagatacagcatgcctccactgttccacacccactacgtgcctgacatcgtgaggtgcgtgccacctttcagagagatcgccttcctggagcctcgcgagattaccctgcctgaggccaaggacaagctgagccagcagattctggaactgttcgagacttgccaacaacaaattagcgacctgaagaagaaggagctgtgcagaacccagctgcagcgcgagatccagctgctgttccctcagagcagactgttcctggtgggcagcagcctgaacggcttcggcaccagaagcagcgacggcgacctgtgcctggtggtgaaggaggagccttgcttcttccaagtaaatcagaagaccgaggccagacacatcctgaccctggtgcacaagcacttctgcaccagactgtccggctacatcgagcgccctcagcttataagagccaaggtgcctatcgtgaagttcagagacaaggtgagctgcgtggagttcgatctgaacgtgaacaacatcgtgggcatcagaaacaccttcctgctgagaacctacgcctacctggagaacagagtcagacctctggtgctggtgatcaagaagtgggcttctcatcaccagatcaacgacgcctcccgcggtactctgtctagctacagcctcgtactgatggtgctgcactacctgcagactctccctgaacctatcctgcctagtctgcagaagatctaccctgagagcttcagccctgccatccaattgcatctcgttcaccaggccccttgcaacgttcctccttatttgagcaagaacgagagcaacctgggagatctcctgctgggcttcctgaagtactacgccaccgaattcgattggaacagccagatgatcagcgtgagagaagcaaaggccatccctagacctgacggcatcgagtggagaaacaagtacatctgtgtggaggaacctttcgacggcaccaacaccgcccgcgcagtacacgagaagcagaagttcgacatgatcaaggaccagttcctcaagagctggcacagactgaagaacaagagagacttgaatagcatcttgccggtgagagccgccgtgctgaagaga

SEQ ID NO: 80 - protein, 193 aa, Artificial Sequence
  source: /note=“Description of Artificial Sequence Synthetic polypeptide”
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 81 - DNA, 579 bp, Artificial Sequence
  source: /note=“Description of Artificial Sequence Synthetic polynucleotide”
atgggagtgcacgagtgtcccgcgtggttgtggttgctgctgtcgctcttgagcctcccactgggactgcctgtgctgggggcaccacccagattgatctgcgactcacgggtacttgagaggtaccttcttgaagccaaagaagccgaaaacatcacaaccggatgcgccgagcactgctccctcaatgagaacattactgtaccggatacaaaggtcaatttctatgcatggaagagaatggaagtaggacagcaggccgtcgaagtgtggcaggggctcgcgcttttgtcggaggcggtgttgcggggtcaggccctcctcgtcaactcatcacagccgtgggagcccctccaacttcatgtcgataaagcggtgtcggggctccgcagcttgacgacgttgcttcgggctctgggcgcacaaaaggaggctatttcgccgcctgacgcggcctccgcggcacccctccgaacgatcaccgcggacacgtttaggaagctttttagagtgtacagcaatttcctccgcggaaagctgaaattgtatactggtgaagcgtgtaggacaggggatcgc

SEQ ID NO: 82 - protein, 284 aa, Artificial Sequence
  source: /note=“Description of Artificial Sequence Synthetic polypeptide”
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKRSRDISHGFPPAVAAQDDGTLPMSCAQESGMDRHPAACASARINV

SEQ ID NO: 83 - DNA, 852 bp, Artificial Sequence
  source: /note=“Description of Artificial Sequence Synthetic polynucleotide”
atggtgtccaagggtgaggaattgtttaccggggtggtgcctattctcgtcgaacttgacggggatgtgaatggacacaagttttcggtatccggagaaggagagggtgacgccacatacggaaagcttacactcaaattcatctgtacgacggggaaactgcccgtaccctggcctacgctcgtaaccacgctgacttatggagtgcagtgctttagcagataccccgaccatatgaagcagcacgacttcttcaagtcggcgatgcccgaggggtacgtgcaagagaggaccattttcttcaaagacgatggcaattacaaaacacgcgcagaagtcaagtttgagggcgatactctggtcaatcggatcgaattgaagggaatcgatttcaaagaagatggaaacatccttggccataagctcgagtacaactataactcgcataatgtctatatcatggctgacaagcagaaaaacggtatcaaagtcaactttaagatccgacacaatattgaggacggttcggtgcagcttgcggaccactatcaacagaatacgccgattggggatggtccggtccttttgccggataaccattatctctcaacccagtcagccctgagcaaagatccaaacgagaagagggaccacatggtcttgctcgaattcgtgacagcggcagggatcactctgggaatggacgagttgtacaagagatctcgagatatcagccatggcttcccgccggcggtggcggcgcaggatgatggcacgctgcccatgtcttgtgcccaggagagcgggatggaccgtcaccctgcagcctgtgcttctgctaggatcaatgtg

SEQ ID NO: 84 - RNA, 100 nt, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 85 - protein, 550 aa, Artificial Sequence
  source: /note=“Description of Artificial Sequence Synthetic polypeptide”
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 86 - DNA, 1650 bp, Artificial Sequence
  source: /note=“Description of Artificial Sequence Synthetic polynucleotide”
atggaagatgcgaagaacatcaagaagggacctgccccgttttaccctttggaggacggtacagcaggagaacagctccacaaggcgatgaaacgctacgccctggtccccggaacgattgcgtttaccgatgcacatattgaggtagacatcacatacgcagaatacttcgaaatgtcggtgaggctggcggaagcgatgaagagatatggtcttaacactaatcaccgcatcgtggtgtgttcggagaactcattgcagtttttcatgccggtccttggagcacttttcatcggggtcgcagtcgcgccagcgaacgacatctacaatgagcgggaactcttgaatagcatgggaatctcccagccgacggtcgtgtttgtctccaaaaaggggctgcagaaaatcctcaacgtgcagaagaagctccccattattcaaaagatcatcattatggatagcaagacagattaccaagggttccagtcgatgtatacctttgtgacatcgcatttgccgccagggtttaacgagtatgacttcgtccccgagtcatttgacagagataaaaccatcgcgctgattatgaattcctcgggtagcaccggtttgccaaagggggtggcgttgccccaccgcactgcttgtgtgcggttctcgcacgctagggatcctatctttggtaatcagatcattcccgacacagcaatcctgtccgtggtaccttttcatcacggttttggcatgttcacgactctcggctatttgatttgcggtttcagggtcgtacttatgtatcggttcgaggaagaactgtttttgagatccttgcaagattacaagatccagtcggccctccttgtgccaacgcttttctcattctttgcgaaatcgacacttattgataagtatgacctttccaatctgcatgagattgcctcagggggagcgccgcttagcaaggaagtcggggaggcagtggccaagcgcttccaccttcccggaattcggcagggatacgggctcacggagacaacatccgcgatccttatcacgcccgagggtgacgataagccgggagccgtcggaaaagtggtccccttctttgaagccaaggtcgtagacctcgacacgggaaaaaccctcggagtgaaccagaggggcgagctctgcgtgagagggccgatgatcatgtcaggttacgtgaataaccctgaagcgacgaatgcgctgatcgacaaggatgggtggttgcattcgggagacattgcctattgggatgaggatgagcacttctttatcgtagatcgacttaagagcttgatcaaatacaaaggctatcaggtagcgcctgccgagctcgagtcaatcctgctccagcaccccaacattttcgacgccggagtggccgggttgcccgatgacgacgcgggtgagctgccagcggccgtggtagtcctcgaacatgggaaaacaatgaccgaaaaggagatcgtggactacgtagcatcacaagtgacgactgcgaagaaactgaggggaggggtagtctttgtggacgaggtcccgaaaggcttgactgggaagcttgacgctcgcaaaatccgggaaatcctgattaaggcaaagaaaggcgggaaaatcgctgtc

SEQ ID NO: 87 - protein, 66 aa, Artificial Sequence
  source: /note=“Description of Artificial Sequence Synthetic polypeptide”
GSGEGRGSLLTCGDVEENPGPGSGATNFSLLKQAGDVEENPGPGSGQCTNYALLKLAGDVESNPGP

SEQ ID NO: 88 - DNA, 198 bp, Artificial Sequence
  source: /note=“Description of Artificial Sequence Synthetic polynucleotide”
ggctctggcgaaggccggggtagcctgctgacctgcggcgacgtggaggagaaccctggacctggatccggcgccaccaacttcagcctgctcaagcaggccggagacgtggaagagaatccaggcccaggcagcggtcagtgcaccaactacgccctgctgaagctggccggcgacgtagagagcaaccccggcccc

SEQ ID NO: 89 - protein, 21 aa, Artificial Sequence
  source: /note=“Description of Artificial Sequence Synthetic peptide”
  VARIANT (1)..(3): /replace=“ ”
  SITE (1)..(21): /note=“Variant residues given in the sequence have no preference with respect to those in the annotations for variant positions”
GSGEGRGSLLTCGDVEENPGP

SEQ ID NO: 90 - protein, 22 aa, Artificial Sequence
  source: /note=“Description of Artificial Sequence Synthetic peptide”
  VARIANT (1)..(3): /replace=“ ”
  SITE (1)..(22): /note=“Variant residues given in the sequence have no preference with respect to those in the annotations for variant positions”
GSGATNFSLLKQAGDVEENPGP

SEQ ID NO: 91 - protein, 23 aa, Artificial Sequence
  source: /note=“Description of Artificial Sequence Synthetic peptide”
  VARIANT (1)..(3): /replace=“ ”
  SITE (1)..(23): /note=“Variant residues given in the sequence have no preference with respect to those in the annotations for variant positions”
GSGQCTNYALLKLAGDVESNPGP

SEQ ID NO: 92 - protein, 25 aa, Artificial Sequence
  source: /note=“Description of Artificial Sequence Synthetic peptide”
  VARIANT (1)..(3): /replace=“ ”
  SITE (1)..(25): /note=“Variant residues given in the sequence have no preference with respect to those in the annotations for variant positions”
GSGVKQTLNFDLLKLAGDVESNPGP

SEQ ID NO: 93 - protein, 1215 aa, Artificial Sequence
  source: /note=“Description of Artificial Sequence Synthetic polypeptide”
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAVGSGEGRGSLLTCGDVEENPGPGSGATNFSLLKQAGDVEENPGPGSGQCTNYALLKLAGDVESNPGPASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSDPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSIANKATPQMAQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKFIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 94 - DNA, 3645 bp, Artificial Sequence
  source: /note=“Description of Artificial Sequence Synthetic polynucleotide”
atggaggacgccaagaacatcaagaaggggcccgctcccttctacccgctggaggacggcaccgctggcgagcagctgcacaaggccatgaagaggtacgcactggtgcccggcacaatcgccttcaccgacgcccacatcgaggtggacatcacctacgccgagtacttcgagatgagcgtgcggctggccgaagccatgaagcggtacggcctgaacaccaaccaccgcatcgtggtgtgctccgagaactccctgcagttcttcatgcccgttctgggcgccctgttcattggggtggccgtggcaccagccaacgacatctacaacgagagggagctgctgaacagcatgggcatcagccagcccaccgtggtgttcgtgtccaagaagggcctgcagaagatcctgaacgtgcagaagaagctgcccatcatccagaagatcatcatcatggacagcaagaccgactaccagggcttccagtccatgtacaccttcgtgaccagccacctgcctcccggcttcaacgagtacgacttcgtgcccgagtccttcgacagggacaagaccatcgccctgatcatgaactccagcggaagcaccggcctgcccaagggtgtggctctaccccaccggaccgcctgtgtacggttcagccacgcccgtgaccccatcttcgggaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagtccgccctgctggtgcccaccctgttctccttcttcgccaagtccaccctgatcgacaagtacgacctgagcaacctgcacgagattgcctctggcggcgcacctctgtccaaggaggtcggggaggccgtagccaagcggttccacctgcccggcatccggcaaggctacggcctgaccgagaccaccagcgccatcctgatcacccctgagggcgacgacaagcccggcgcagtggggaaggtggtgccattcttcgaggccaaggtggtggacctggacaccggcaagaccctgggcgtgaaccagcggggcgagctgtgcgtgcggggacccatgatcatgtccggctacgtgaacaaccccgaggccaccaacgccctgattgacaaggacgggtggctgcacagcggcgacatcgcctactgggacgaggacgagcacttcttcatcgtggaccgcctgaagtccctgatcaagtacaaggggtaccaggtggctcccgccgagctggagtccatcctgctgcagcaccccaacatctttgacgccggcgttgccggactgcctgacgacgacgctggggaactccccgcagctgtggtggtgctggagcacggcaagaccatgaccgagaaggagatcgtggactacgtcgcctcccaggtgaccaccgccaagaagttacgcggcggcgtcgtgttcgtggacgaggtgcccaagggcctgaccggcaagctggacgcccgcaagatccgggagatcctgatcaaggccaagaagggcgggaagatcgccgtgggctctggcgaaggccggggtagcctgctgacctgcggcgacgtggaggagaaccctggacctggatccggcgccaccaacttcagcctgctcaagcaggccggagacgtggaagagaatccaggcccaggcagcggtcagtgcaccaactacgccctgctgaagctggccggcgacgtagagagcaaccccggccccgccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggtggcggaggcagcgaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcgccaacaaggcgacccctcagatggcgcagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaagttcataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 95 - protein, 1783 aa, Artificial Sequence
  source: /note=“Description of Artificial Sequence Synthetic polypeptide”
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAVGSGEGRGSLLTCGDVEENPGPGSGATNFSLLKQAGDVEENPGPGSGQCTNYALLKLAGDVESNPGPASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGGGGSSFTLTNKNVIFVAGLGGIGLDTSKELLKRDPVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAELQLSAGRYHYQLVWCQK

SEQ ID NO: 96 - DNA, 5349 bp, Artificial Sequence
  source: /note=“Description of Artificial Sequence Synthetic polynucleotide”
atggaggacgccaagaacatcaagaaggggcccgctcccttctacccgctggaggacggcaccgctggcgagcagctgcacaaggccatgaagaggtacgcactggtgcccggcacaatcgccttcaccgacgcccacatcgaggtggacatcacctacgccgagtacttcgagatgagcgtgcggctggccgaagccatgaagcggtacggcctgaacaccaaccaccgcatcgtggtgtgctccgagaactccctgcagttcttcatgcccgttctgggcgccctgttcattggggtggccgtggcaccagccaacgacatctacaacgagagggagctgctgaacagcatgggcatcagccagcccaccgtggtgttcgtgtccaagaagggcctgcagaagatcctgaacgtgcagaagaagctgcccatcatccagaagatcatcatcatggacagcaagaccgactaccagggcttccagtccatgtacaccttcgtgaccagccacctgcctcccggcttcaacgagtacgacttcgtgcccgagtccttcgacagggacaagaccatcgccctgatcatgaactccagcggaagcaccggcctgcccaagggtgtggctctaccccaccggaccgcctgtgtacggttcagccacgcccgtgaccccatcttcgggaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagtccgccctgctggtgcccaccctgttctccttcttcgccaagtccaccctgatcgacaagtacgacctgagcaacctgcacgagattgcctctggcggcgcacctctgtccaaggaggtcggggaggccgtagccaagcggttccacctgcccggcatccggcaaggctacggcctgaccgagaccaccagcgccatcctgatcacccctgagggcgacgacaagcccggcgcagtggggaaggtggtgccattcttcgaggccaaggtggtggacctggacaccggcaagaccctgggcgtgaaccagcggggcgagctgtgcgtgcggggacccatgatcatgtccggctacgtgaacaaccccgaggccaccaacgccctgattgacaaggacgggtggctgcacagcggcgacatcgcctactgggacgaggacgagcacttcttcatcgtggaccgcctgaagtccctgatcaagtacaaggggtaccaggtggctcccgccgagctggagtccatcctgctgcagcaccccaacatctttgacgccggcgttgccggactgcctgacgacgacgctggggaactccccgcagctgtggtggtgctggagcacggcaagaccatgaccgagaaggagatcgtggactacgtcgcctcccaggtgaccaccgccaagaagttacgcggcggcgtcgtgttcgtggacgaggtgcccaagggcctgaccggcaagctggacgcccgcaagatccgggagatcctgatcaaggccaagaagggcgggaagatcgccgtgggctctggcgaaggccggggtagcctgctgacctgcggcgacgtggaggagaaccctggacctggatccggcgccaccaacttcagcctgctcaagcaggccggagacgtggaagagaatccaggcccaggcagcggtcagtgcaccaactacgccctgctgaagctggccggcgacgtagagagcaaccccggccccgccagcaacttcacccagttcgtgctggtggacaacggcggtaccggagacgtgaccgtggccccttctaacttcgccaacggcatcgccgagtggatcagcagcaacagcagaagccaggcctacaaggtgacctgcagcgtgagacagagcagcgcccagaacagaaagtacaccatcaaggtggaggtgcctaagggcgcctggagaagctacctgaacatggagctgaccatccctatcttcgccaccaacagcgactgcgagctgatcgtgaaggccatgcagggcctgctgaaggacggcaaccctatccctagcgccatcgccgccaattcaggcatctacggaggcggtggaagctcgttcacgctaacgaacaagaacgtcatcttcgtagcgggacttggcggtatcggcctagacacgtcgaaggaactactaaagcgtgacccggtagtcctccaacgtcgcgattgggagaacccgggcgtaacgcaactaaaccgtcttgcggcgcacccgccgtttgcgtcgtggcgtaactcggaggaggcgcgaacggatcgtccgtcgcaacaactacgttcgctcaacggggagtggcgcttcgcgtggttcccggcgccggaggcggtaccggagtcgtggctcgagtgcgatctaccggaggcggacacggtcgtcgtaccgtcgaactggcaaatgcacggttacgacgcgccgatatacacgaacgtcacgtacccgataacggtaaacccgccgttcgtcccgacggagaacccgacggggtgctactcgctaacgttcaacgttgacgagtcgtggttgcaagagggtcaaacgcgtatcatattcgacggtgtaaactcggcgttccacctgtggtgcaacgggcgctgggtagggtacggccaagactcgcgtctaccgtcggagttcgacctatcggcgttcctacgagcgggtgagaaccggctagcggtcatggtcctacgttggtcggacggttcgtacctcgaggaccaagacatgtggcgaatgtcgggtatcttccgcgacgtatcgctcctacacaagccgacgacgcaaatctcggacttccacgtcgcgacgcgtttcaacgacgatttctcgcgggcagtcctagaggcggaggtccaaatgtgcggggagctacgtgactacctccgtgtcacggtatcgctctggcaaggtgagacgcaagtagcgtcgggtacggcgccgttcggcggtgagatcatcgacgagcgtggtgggtacgcggaccgtgtaacgctacgtctaaacgtcgagaacccgaagctctggtcggcggagatcccgaacctataccgtgcggtcgtcgagctacatacggcggacgggacgctaatagaggcggaagcgtgcgacgtcgggtttcgagaggttcgtatagagaacgggctgctacttctaaacgggaagccgttgctcatacgtggtgtcaaccgtcacgagcaccacccgctacacggtcaagtaatggacgagcaaacgatggtacaagacatcctactaatgaagcagaacaacttcaacgcggtacgctgttcgcattacccgaaccatccgttgtggtacacgctttgcgaccgatacggtctatacgtcgtagacgaggcgaacatagagacgcacgggatggtaccgatgaatcgcctaacggacgacccgcgttggctaccggcgatgtcggagcgagtcacgcgtatggtccaacgggaccgtaaccacccgtcggtaataatctggtcgctaggcaacgaatcggggcacggggcgaaccacgacgcgctataccgttggatcaagtcggtagacccgtcgcgtccggtacaatacgaaggtggcggtgcggacacgacggcgacggacatcatctgcccgatgtacgcgcgcgtcgacgaagaccaaccgttcccggcggtaccgaagtggtcgatcaagaagtggctctcgttgccgggtgaaacgcgtccgttgatactttgcgagtacgcgcacgcgatgggcaactcgttgggtgggttcgcgaagtactggcaggcgttccgtcaatacccgcgtctacagggtgggttcgtctgggactgggtagaccaatcgctaatcaagtacgacgagaacggcaacccgtggtcggcgtacggtggggacttcggggacacgccgaacgaccgccaattctgtatgaacggcctagtcttcgcggaccgaacgccgcacccggcgttgacggaggcgaagcatcaacaacaattcttccaattccgtctatcggggcaaacgatcgaggtaacgtcggagtacttgttccggcactcggacaacgagctactacactggatggtagcactagacggcaagccgctagcgtcgggagaagtccctttggacgtcgcgccgcaaggtaagcaactaatcgagctaccggagctaccgcaaccggagtcggcgggtcaactgtggttgacggtccgtgtcgttcaaccgaacgcgacggcgtggtcggaggcgggtcacatctcggcgtggcagcagtggcgtctagcggagaacctctcggtcacgctaccggcggcgtcgcacgcgataccgcatctaacgacgtcggagatggacttctgcatcgagttggggaacaagaggtggcagttcaaccgtcaatcgggattcctatcgcaaatgtggataggtgacaagaagcaactactaacgccgctacgtgatcagttcacgcgtgctccgctagacaacgacataggtgtttcggaggcgacgcgtatagacccgaacgcgtgggtggagcggtggaaggcggcggggcactaccaagcggaggcggcgctactacagtgcacggcggacacgctagcggacgcggtattgatcacgacggcgcacgcgtggcaacaccaggggaagacgctattcatctcgcgtaagacgtaccgtatcgacggttcgggccaaatggcgatcacggtcgacgtagaggtagcgtcggacacgccgcatccggcgcgcatcggtctaaactgccaactagcgcaagtagcggagcgtgtaaactggctagggctagggccgcaagagaactatccggaccgcctaacggcggcgtgcttcgaccgttgggacctaccgctttcggacatgtataccccgtacgtcttcccgtcggagaacgggttgaggtgcgggacgcgcgagctaaactacgggccgcaccagtggcgaggggacttccaattcaacatatcgcgttactcgcaacaacaactaatggagacgtcgcaccgtcacctactacacgcggaggaggggacgtggctaaacatcgacgggttccacatgggcataggtggggacgactcgtggtcgccgtcggtctcggcggagctccaactctcggcgggtcgttaccattaccaactagtttggtgccagaag

SEQ ID NO: 97 - protein, 1047 aa, Artificial Sequence
  source: /note=“Description of Artificial Sequence Synthetic polypeptide”
MSFTLTNKNVIFVAGLGGIGLDTSKELLKRDPVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAELQLSAGRYHYQLVWCQK

SEQ ID NO: 98 - DNA, 3141 bp, Artificial Sequence
  source: /note=“Description of Artificial Sequence Synthetic polynucleotide”
atgagcttcaccctgaccaacaagaacgtgatcttcgtggccggcctgggcggcatcggcctggacaccagcaaggagctgctgaagcgggaccccgtggtgctgcagcgccgggactgggagaaccccggcgtgacccagctgaacaggctggccgcccatcctccgttcgcctcctggcggaactccgaggaggcccggaccgaccggccctcccagcagctgcggtctctgaacggcgagtggaggttcgcctggttccccgccccagaggccgtgcctgagagctggctggagtgcgacctgcctgaagccgacaccgtggtggtgcctagcaactggcagatgcacggctacgacgcacccatctacaccaacgtgacctaccctatcaccgtgaatccgcctttcgtgcccaccgagaaccccaccggctgctacagcctgaccttcaacgtggacgagagttggctgcaggagggccagacccgcatcatcttcgacggcgtgaacagcgccttccacctgtggtgcaacggccggtgggtgggctacggccaggactcccggctgccctccgagtttgacctgtccgccttcctgcgcgccggcgagaaccggctggccgtgatggtgctgaggtggagcgacggcagctacctggaggaccaggacatgtggcgcatgtccggaatcttccgggacgtgagcctgctgcacaagcccaccacccagatcagcgacttccacgtggccacccggttcaacgacgacttcagccgggccgtgctggaggccgaggtgcagatgtgcggcgagctgcgggactacctgcgcgtgaccgtgagcctgtggcagggcgagacccaggtggcttccggcaccgccccattcggcggcgagatcatcgacgagcggggcggctacgccgaccgggtgaccctgcggctgaacgtggagaaccccaagctgtggagcgccgagatccccaacctgtaccgggccgtggtggagctgcacaccgccgacggcaccctgatcgaggccgaggcctgcgacgtgggcttccgggaggtgaggatcgagaacgggctgctgctgctgaacgggaagccactgctgatcaggggcgtgaaccgccacgagcaccatcccctgcacggccaggtgatggacgagcagaccatggtgcaggacatcctgctgatgaagcagaacaacttcaacgccgtgaggtgctcccactaccccaaccatcccctgtggtacaccctgtgcgaccggtacggcctgtacgtggtggacgaggccaacatcgagacccacgggatggtcccaatgaaccggctgactgacgaccctcgctggctgcccgccatgagcgagcgggtgaccaggatggtgcagcgcgaccggaaccaccccagcgtgatcatctggagcctggggaacgagtccggccacggcgccaaccacgacgccctgtaccgctggatcaagagcgtggacccctcccgccccgtgcagtacgagggcggcggcgccgacaccaccgccaccgacatcatctgccccatgtacgcccgggtggacgaggaccagcccttccctgccgtgcccaagtggagcatcaagaagtggctgagcttgcccggcgagaccagaccgctgatcctgtgcgagtacgcccacgccatgggcaactccctgggcggcttcgccaagtactggcaggccttccggcagtacccgcggctgcagggcggcttcgtgtgggactgggtggaccagtccctgatcaagtacgacgagaacggcaacccctggagcgcctacggcggggacttcggggacacacccaacgaccgccagttctgcatgaacggcctggtgttcgccgaccgcaccccgcaccctgccctgaccgaggccaagcaccagcagcagttcttccagttccggctgagcgggcagaccatcgaggtgaccagcgagtacctgttccggcacagcgacaacgagctgctgcactggatggtggccctggacggcaagcccctggccagcggcgaggtgcctctggacgtggctccccagggcaagcagctgatcgagctgcccgagctgccccagcccgagagcgccggccagctgtggctgaccgtgagggtggtgcagcccaacgccaccgcctggagcgaggccggccacatcagcgcctggcaacagtggcggctggccgagaacctgagcgtgaccctgcccgccgccagccacgccatccctcacctgaccacctccgagatggacttctgcatcgagctgggcaacaagcggtggcagttcaaccggcagtccggcttcctgagccagatgtggatcggcgacaagaagcagctgctgaccccactgcgggaccagttcaccagggccccactggacaacgacatcggcgtgagcgaggccaccagaatcgaccccaacgcctgggtggagcggtggaaggccgccggccactaccaggccgaggccgccctgctgcagtgcaccgccgacaccctggccgacgccgtgctgatcaccaccgcccacgcctggcagcaccaggggaagaccctgttcatcagccggaagacctaccggatcgacgggtccggccagatggccatcaccgtggacgtggaggtggcctccgacacccctcaccccgcccggatcggcctgaactgtcagctggcccaggtggccgagcgggtgaactggctggggctgggcccacaggagaactaccccgaccggctgaccgccgcctgcttcgaccggtgggacctgcccctgagcgacatgtacaccccatacgtgttcccctctgagaacggcctgcggtgcggcacccgggagctgaactacgggccccaccagtggcgcggcgacttccagttcaacatcagccggtacagccagcagcagctgatggagaccagccaccggcacctgctgcacgccgaggagggcacctggctgaacatcgacggcttccacatgggcatcggcggcgacgacagctggtccccatccgtgagcgccgagctgcagctgagcgccggccggtaccactaccagctggtgtggtgccagaag

SEQ ID NO: 99 - RNA, 133 nt, Artificial Sequence
  source: /note=“Description of Artificial Sequence Synthetic polynucleotide”
gcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 100 - RNA, 141 nt, Artificial Sequence
  source: /note=“Description of Artificial Sequence Synthetic polynucleotide”
ugauaauaggcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 101 - RNA, 119 nt, Artificial Sequence
  source: /note=“Description of Artificial Sequence Synthetic polynucleotide”
ugauaauaggcuggagccucgguggccaugcuucuugccccuugggccuccccccagccccuccuccccuuccugcacccguacccccguggucuuugaauaaagucugagugggcggc

SEQ ID NO: 102 - RNA, 142 nt, Artificial Sequence
  source: /note=“Description of Artificial Sequence Synthetic polynucleotide”
ugauaauaggcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 103 - RNA, 164 nt, Artificial Sequence
  source: /note=“Description of Artificial Sequence Synthetic polynucleotide”
ugauaauaguccauaaaguaggaaacacuacagcuggagccucgguggccaugcuucuugccccuugggccuccccccagccccuccuccccuuccugcacccguaccccccgcauuauuacucacgguacgaguggucuuugaauaaagucugagugggcggc

SEQ ID NO: 104 - RNA, 188 nt, Artificial Sequence
  source: /note=“Description of Artificial Sequence Synthetic polynucleotide”
ugauaauaguccauaaaguaggaaacacuacagcuggagccucgguggccaugcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 105 - RNA, 140 nt, Artificial Sequence
  source: /note=“Description of Artificial Sequence Synthetic polynucleotide”
ugauaauaggcuggagccucgguggccaugcuucuugccccuugggccuccccccagccccuccuccccuuccugcacccguacccccaguagugcuuucuacuuuaugguggucuuugaauaaagucugagugggcggc

SEQ ID NO: 106 - RNA, 182 nt, Artificial Sequence
  source: /note=“Description of Artificial Sequence Synthetic polynucleotide”
ugauaauagaguagugcuuucuacuuuauggcuggagccucgguggccaugcuucuugccccuugggccaguagugcuuucuacuuuauguccccccagccccuccuccccuuccugcacccguacccccaguagugcuuucuacuuuaugguggucuuugaauaaagucugagugggcggc

SEQ ID NO: 107 - RNA, 184 nt, Artificial Sequence
  source: /note=“Description of Artificial Sequence Synthetic polynucleotide”
ugauaauagaguagugcuuucuacuuuauggcuggagccucgguggccaugcuucuugccccuugggccuccauaaaguaggaaacacuacauccccccagccccuccuccccuuccugcacccguacccccaguagugcuuucuacuuuaugguggucuuugaauaaagucugagugggcggc

SEQ ID NO: 108 - RNA, 142 nt, Artificial Sequence
  source: /note=“Description of Artificial Sequence Synthetic polynucleotide”
ugauaauaggcuggagccucgguggccaugcuucuugccccuugggccuccccccagccccuccuccccuuccugcacccguacccccaccccuaucacaauuagcauuaaguggucuuugaauaaagucugagugggcggc

SEQ ID NO: 109 - RNA, 188 nt, Artificial Sequence
  source: /note=“Description of Artificial Sequence Synthetic polynucleotide”
ugauaauagaccccuaucacaauuagcauuaagcuggagccucgguggccaugcuucuugccccuugggccaccccuaucacaauuagcauuaauccccccagccccuccuccccuuccugcacccguacccccaccccuaucacaauuagcauuaaguggucuuugaauaaagucugagugggcggc

SEQ ID NO: 110 - RNA, 188 nt, Artificial Sequence
  source: /note=“Description of Artificial Sequence Synthetic polynucleotide”
ugauaauagaccccuaucacaauuagcauuaagcuggagccucgguggccaugcuucuugccccuugggccuccauaaaguaggaaacacuacauccccccagccccuccuccccuuccugcacccguacccccaccccuaucacaauuagcauuaaguggucuuugaauaaagucugagugggcggc

SEQ ID NO: 111 - RNA, 142 nt, Artificial Sequence
  source: /note=“Description of Artificial Sequence Synthetic polynucleotide”
ugauaauaguccauaaaguaggaaacacuacagcuggagccucgguggccaugcuucuugccccuugggccuccccccagccccuccuccccuuccugcacccguacccccguggucuuugaauaaagucugagugggcggc

SEQ ID NO: 112 - RNA, 142 nt, Artificial Sequence
  source: /note=“Description of Artificial Sequence Synthetic polynucleotide”
ugauaauaggcuggagccucgguggcuccauaaaguaggaaacacuacacaugcuucuugccccuugggccuccccccagccccuccuccccuuccugcacccguacccccguggucuuugaauaaagucugagugggcggc

SEQ ID NO: 113 - RNA, 142 nt, Artificial Sequence
  source: /note=“Description of Artificial Sequence Synthetic polynucleotide”
ugauaauaggcuggagccucgguggccaugcuucuugccccuugggccuccauaaaguaggaaacacuacauccccccagccccuccuccccuuccugcacccguacccccguggucuuugaauaaagucugagugggcggc

SEQ ID NO: 114 - RNA, 181 nt, Artificial Sequence
  source: /note=“Description of Artificial Sequence Synthetic polynucleotide”
ugauaauagaguagugcuuucuacuuuauggcuggagccucgguggccaugcuucuugccccuugggccaguagugcuuucuacuuuauguccccccagccccucuccccuuccugcacccguacccccaguagugcuuucuacuuuaugguggucuuugaauaaagucugagugggcggc

SEQ ID NO: 115 - RNA, 142 nt, Artificial Sequence
  source: /note=“Description of Artificial Sequence Synthetic polynucleotide”
ugauaauaggcuggagccucgguggccaugcuucuugccccuuccauaaaguaggaaacacuacaugggccuccccccagccccuccuccccuuccugcacccguacccccguggucuuugaauaaagucugagugggcggc

SEQ ID NO: 116 - RNA, 142 nt, Artificial Sequence
  source: /note=“Description of Artificial Sequence Synthetic polynucleotide”
ugauaauaggcuggagccucgguggccaugcuucuugccccuugggccuccccccaguccauaaaguaggaaacacuacaccccuccuccccuuccugcacccguacccccguggucuuugaauaaagucugagugggcggc

SEQ ID NO: 117 - RNA, 142 nt, Artificial Sequence
  source: /note=“Description of Artificial Sequence Synthetic polynucleotide”
ugauaauaggcuggagccucgguggccaugcuucuugccccuugggccuccccccagccccuccuccccuucuccauaaaguaggaaacacuacacugcacccguacccccguggucuuugaauaaagucugagugggcggc

SEQ ID NO: 118 - RNA, 142 nt, Artificial Sequence
  source: /note=“Description of Artificial Sequence Synthetic polynucleotide”
ugauaauaggcuggagccucgguggccaugcuucuugccccuugggccuccccccagccccuccuccccuuccugcacccguacccccguggucuuugaauaaaguuccauaaaguaggaaacacuacacugagugggcggc

SEQ ID NO: 119 - RNA, 164 nt, Artificial Sequence
  source: /note=“Description of Artificial Sequence Synthetic polynucleotide”
ugauaauaguccauaaaguaggaaacacuacagcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 120 - RNA, 119 nt, Artificial Sequence
  source: /note=“Description of Artificial Sequence Synthetic polynucleotide”
ugauaauaggcuggagccucgguggccuagcuucuugccccuugggccuccccccagccccuccuccccuuccugcacccguacccccguggucuuugaauaaagucugagugggcggc

SEQ ID NO: 121 - RNA, 142 nt, Artificial Sequence
  source: /note=“Description of Artificial Sequence Synthetic polynucleotide”
ugauaauaggcuggagccucgguggccuagcuucuugccccuugggccuccccccagccccuccuccccuuccugcacccguacccccuccauaaaguaggaaacacuacaguggucuuugaauaaagucugagugggcggc

SEQ ID NO: 122 - RNA, 141 nt, Artificial Sequence
  source: /note=“Description of Artificial Sequence Synthetic polynucleotide”
ugauaauaggcuggagccucgguggccuagcuucuugccccuugggccuccccccagccccuccuccccuuccugcacccguaccccccgcauuauuacucacgguacgaguggucuuugaauaaagucugagugggcggc

SEQ ID NO: 123 - RNA, 188 nt, Artificial Sequence
  source: /note=“Description of Artificial Sequence Synthetic polynucleotide”
ugauaauaguccauaaaguaggaaacacuacagcuggagccucgguggccuagcuucuugccccuugggccuccauaaaguaggaaacacuacauccccccagccccuccuccccuuccugcacccguacccccuccauaaaguaggaaacacuacaguggucuuugaauaaagucugagugggcggc

SEQ ID NO: 124 - RNA, 142 nt, Artificial Sequence
  source: /note=“Description of Artificial Sequence Synthetic polynucleotide”
ugauaauaguccauaaaguaggaaacacuacagcuggagccucgguggccuagcuucuugccccuugggccuccccccagccccuccuccccuuccugcacccguacccccguggucuuugaauaaagucugagugggcggc

SEQ ID NO: 125 - RNA, 142 nt, Artificial Sequence
  source: /note=“Description of Artificial Sequence Synthetic polynucleotide”
ugauaauaggcuggagccucgguggcuccauaaaguaggaaacacuacacuagcuucuugccccuugggccuccccccagccccuccuccccuuccugcacccguacccccguggucuuugaauaaagucugagugggcggc

SEQ ID NO: 126 - RNA, 142 nt, Artificial Sequence
  source: /note=“Description of Artificial Sequence Synthetic polynucleotide”
ugauaauaggcuggagccucgguggccuagcuucuugccccuugggccuccauaaaguaggaaacacuacauccccccagccccuccuccccuuccugcacccguacccccguggucuuugaauaaagucugagugggcggc

SEQ ID NO: 127 - RNA, 142 nt, Artificial Sequence
  source: /note=“Description of Artificial Sequence Synthetic polynucleotide”
ugauaauaggcuggagccucgguggccuagcuucuugccccuugggccuccccccagccccuccuccccuuccugcacccguacccccaccccuaucacaauuagcauuaaguggucuuugaauaaagucugagugggcggc

SEQ ID NO: 128 - RNA, 188 nt, Artificial Sequence
  source: /note=“Description of Artificial Sequence Synthetic polynucleotide”
ugauaauagaccccuaucacaauuagcauuaagcuggagccucgguggccuagcuucuugccccuugggccaccccuaucacaauuagcauuaauccccccagccccuccuccccuuccugcacccguacccccaccccuaucacaauuagcauuaaguggucuuugaauaaagucugagugggcggc

SEQ ID NO: 129 - RNA, 188 nt, Artificial Sequence
  source: /note=“Description of Artificial Sequence Synthetic polynucleotide”
ugauaauagaccccuaucacaauuagcauuaagcuggagccucgguggccuagcuucuugccccuugggccuccauaaaguaggaaacacuacauccccccagccccuccuccccuuccugcacccguacccccaccccuaucacaauuagcauuaaguggucuuugaauaaagucugagugggcggc

SEQ ID NO: 130 - RNA, 141 nt, Artificial Sequence
  source: /note=“Description of Artificial Sequence Synthetic polynucleotide”
ugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 131 - RNA, 141 nt, Artificial Sequence
  source: /note=“Description of Artificial Sequence Synthetic polynucleotide”
ugauaauaggcuggagccucgguggccuagcuucuugccccuugggccuccccccagccccuccuccccuuccugcacccguaccccccaaacaccauugucacacuccaguggucuuugaauaaagucugagugggcggc

SEQ ID NO: 132 - RNA, 141 nt, Artificial Sequence; identical to SEQ ID NO: 122
  source: /note=“Description of Artificial Sequence Synthetic polynucleotide”

SEQ ID NO: 133 - RNA, 47 nt, Artificial Sequence
  source: /note=“Description of Artificial Sequence Synthetic oligonucleotide”
gggaaauaagagagaaaagaagaguaagaagaaauauaagagccacc

SEQ ID NO: 134 - RNA, 70 nt, Artificial Sequence
  source: /note=“Description of Artificial Sequence Synthetic oligonucleotide”
gggaaauaagaguccauaaaguaggaaacacuacaagaaaagaagaguaagaagaaauauaagagccacc

SEQ ID NO: 135 - RNA, 70 nt, Artificial Sequence
  source: /note=“Description of Artificial Sequence Synthetic oligonucleotide”
gggaaauaagagagaaaagaagaguaauccauaaaguaggaaacacuacagaagaaauauaagagccacc

SEQ ID NO: 136 - RNA, 70 nt, Artificial Sequence
  source: /note=“Description of Artificial Sequence Synthetic oligonucleotide”
gggaaauaagagagaaaagaagaguaagaagaaauauaauccauaaaguaggaaacacuacagagccacc

SEQ ID NO: 137 - RNA, 47 nt, Artificial Sequence
  source: /note=“Description of Artificial Sequence Synthetic oligonucleotide”
aggaaauaagagagaaaagaagaguaagaagaaauauaagagccacc

SEQ ID NO: 138 - RNA, 57 nt, Artificial Sequence
  source: /note=“Description of Artificial Sequence Synthetic oligonucleotide”
gggaaauaagagagaaaagaagaguaagaagaaauauaagaccccggcgccgccacc

SEQ ID NO: 139 - RNA, 57 nt, Artificial Sequence
  source: /note=“Description of Artificial Sequence Synthetic oligonucleotide”
aggaaauaagagagaaaagaagaguaagaagaaauauaagaccccggcgccgccacc

SEQ ID NO: 140 - RNA, 47 nt, Artificial Sequence
  source: /note=“Description of Artificial Sequence Synthetic oligonucleotide”
gggagaucagagagaaaagaagaguaagaagaaauauaagagccacc

SEQ ID NO: 141 - RNA, 42 nt, Artificial Sequence
  source: /note=“Description of Artificial Sequence Synthetic oligonucleotide”
gggagacaagcuuggcauuccgguacuguugguaaagccacc

SEQ ID NO: 142 - RNA, 47 nt, Artificial Sequence
  source: /note=“Description of Artificial Sequence Synthetic oligonucleotide”
gggaauuaacagagaaaagaagaguaagaagaaauauaagagccacc

SEQ ID NO: 143 - RNA, 47 nt, Artificial Sequence
  source: /note=“Description of Artificial Sequence Synthetic oligonucleotide”
gggaaauuagacagaaaagaagaguaagaagaaauauaagagccacc

SEQ ID NO: 144 - RNA, 47 nt, Artificial Sequence
  source: /note=“Description of Artificial Sequence Synthetic oligonucleotide”
gggaaauaagagaguaaagaacaguaagaagaaauauaagagccacc

SEQ ID NO: 145 - RNA, 47 nt, Artificial Sequence
  source: /note=“Description of Artificial Sequence Synthetic oligonucleotide”
gggaaaaaagagagaaaagaagacuaagaagaaauauaagagccacc

SEQ ID NO: 146 - RNA, 47 nt, Artificial Sequence
  source: /note=“Description of Artificial Sequence Synthetic oligonucleotide”
gggaaauaagagagaaaagaagaguaagaagauauauaagagccacc

SEQ ID NO: 147 - RNA, 47 nt, Artificial Sequence
  source: /note=“Description of Artificial Sequence Synthetic oligonucleotide”
gggaaauaagagacaaaacaagaguaagaagaaauauaagagccacc

SEQ ID NO: 148 - RNA, 47 nt, Artificial Sequence
  source: /note=“Description of Artificial Sequence Synthetic oligonucleotide”
gggaaauuagagaguaaagaacaguaaguagaauuaaaagagccacc

SEQ ID NO: 149 - RNA, 47 nt, Artificial Sequence
  source: /note=“Description of Artificial Sequence Synthetic oligonucleotide”
gggaaauaagagagaauagaagaguaagaagaaauauaagagccacc

SEQ ID NO: 150 - RNA, 47 nt, Artificial Sequence
  source: /note=“Description of Artificial Sequence Synthetic oligonucleotide”
gggaaauaagagagaaaagaagaguaagaagaaaauuaagagccacc

SEQ ID NO: 151 - RNA, 47 nt, Artificial Sequence
  source: /note=“Description of Artificial Sequence Synthetic oligonucleotide”
gggaaauaagagagaaaagaagaguaagaagaaauuuaagagccacc

SEQ ID NO: 152 - RNA, 92 nt, Artificial Sequence
  source: /note=“Description of Artificial Sequence Synthetic oligonucleotide”
ucaagcuuuuggacccucguacagaagcuaauacgacucacuauagggaaauaagagagaaaagaagaguaagaagaaauauaagagccacc

SEQ ID NO: 153 - RNA, 120 nt, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 154 - DNA, 925 bp, Artificial Sequence
  source: /note=“Description of Artificial Sequence Synthetic polynucleotide”
tgataataggcgcgatgatcacacgcgcacgcgaagcttgtgttcgcacggcaagcagcagctgccgtggaggcttccacggcgaaatgtccacgaatacgggacaaataattgatatagggccggcccgtaagagtactacgggatgattctcttgacttaagtgccggaccactaggccggcaacggacagacaaagaggctctgggactaccaagcccagtcacacggtacttcaaggcaccgggaacagtagtcccggagtgttgacttatagaagtagtcccattattaaagggacttcgggacttcccttctcgctcccgaccagtaggcgggaaatgcgccctcccgtactgtcctgcaccattagggcaggttctttgaggctctctcgctcgtgtatgaatacaacacgtgtgcgtcctgtcccctcgtgactcaggactaccgagtcacagcaaacttaaaggatcgcacggacgaacacgccgtgacatagctaatcagacaatggcaccagtaccaacggtgcacgttgacactcgaacgtgattgacaggattaccgtcaactcttccactagcccgttactggcaaggattacctgccactgttttgtcttaaccggttgacgtatcaacagaacgtctggtagcagccacgcttactgggccattaataaaggcccaaggaacttaaagccagtgcccggcactatcaaggccgggcatcattgattcgacgccggacggaggagcaccccgtcgggcatcgcggccctgcgggtgctaaacagtagttagcagacatttagcgccggtttgaagcccacgagtacggggcttagcagctccagatacacggcgggaactactaagtcccgtaaactctccactatcgccgggccaatcaatagatggcc

SEQ ID NO: 155 - protein, 478 aa, Artificial Sequence
  source: /note=“Description of Artificial Sequence Synthetic polypeptide”
DPTRLQGINCGPDFTPSFANLGRTTLSTRGPPRGGPGGELPRGPAGLGPRRSQQGPRKEPRKIIATVLMTEDIKLNKAEKAWKPSSKRTAADKDRGEEDADGSKTQDLFRRVRSILNKLTPQMFQQLMKQVTQLAIDTEERLKGVIDLIFEKAISEPNFSVAYANMCRCLMALKVPTTEKPTVTVNFRKLLLNRCQKEFEKDKDDDEVFEKKQKEMDEAATAEERGRLKEELEEARDIARRRSLGNIKAIGELFKLKMLTEAIMHDCVVKLLKNHDEESLECLCRLLTTIGKDLDFEKAKPRMDQYFNQMEKIIKEKKTSSRIRFMLQDVLDLRGSNWVPRRGDQGPKTIDQIHKEAEMEEHREHIKVQQLMAKGSDKRRGGPPGPPISRGLPLVDDGGWNTVPISKGSRPIDTSRLTKITKPGSIDSNNQLFAPGGRLSWGKGSSGGSGAKPSDAASEAARPATSTLNRFSALQQAV

SEQ ID NO: 156 - DNA, 1434 bp, Artificial Sequence
  source: /note=“Description of Artificial Sequence Synthetic polynucleotide”
gaccctaccagactgcagggcatcaactgcggccctgacttcactccttctttcgcaaacctgggcagaaccaccctgagcaccagaggccctcctagaggtggtcccggcggagaactccccaggggtcctgccggcctgggccctagacgctcccagcaaggtcctagaaaggagccaaggaagatcatcgccaccgtgctgatgaccgaggacatcaagctgaacaaagctgagaaggcctggaagcctagcagcaagagaaccgccgccgacaaggacagaggcgaggaggacgccgacggatccaagacccaggacctgttcagaagagtgagaagcatcctcaacaagctgacccctcagatgttccagcagctgatgaagcaggtgacgcagctcgccatcgacaccgaggagagactgaagggcgtgatcgacctgatctttgagaaggctatctcagagcctaacttcagcgtggcctacgccaacatgtgccgttgcctgatggcattgaaggtgccaaccaccgagaagcctactgtgaccgtcaatttccgtaaactgctgctgaaccggtgccagaaagagttcgagaaggataaggacgacgacgaggtcttcgagaagaaacagaaagaaatggacgaggccgccaccgcagaggaaaggggccgattaaaggaggagctggaggaggccagagacatcgccagacggcgttctctgggcaacatcaaggcgataggtgagctgttcaagctaaagatgctcaccgaggccataatgcacgactgcgtggtgaagctactgaagaaccacgacgaagaaagcctggagtgcctgtgcagactgctgaccaccatcggcaaggacctggacttcgagaaggcaaagcctcgaatggaccagtacttcaaccagatggagaagattatcaaggagaagaagaccagcagcagaatcagattcatgctgcaggacgtactggacctgcgcggaagcaactgggtgccaaggagaggggaccaaggaccaaagaccatcgaccagatccacaaggaagcggagatggaggagcacagagagcacataaaggtgcagcagcttatggccaagggcagcgacaagcgaagaggcggcccgcccggacctcctatcagcagaggccttcctctggtagacgacggcggctggaacaccgtgcctatctctaagggctccagacctatcgacacttcccgtcttaccaagatcaccaagccaggatctattgacagcaacaaccagctgttcgccccaggaggaagacttagctggggcaagggaagttccggcggatccggcgccaagccttccgacgccgccagcgaggctgccagacctgccaccagcaccttgaaccgcttttccgctctgcagcaagctgtg

SEQ ID NO: 157 - RNA, 9 nt, Artificial Sequence
  source: /note=“Description of Artificial Sequence Synthetic oligonucleotide”
ccrccaugg